(19) United States
(12) Patent Application Publication
(10) Pub. No.: US 2023/0416831 A1
(43) Pub. Date: Dec. 28, 2023

(54) COMPREHENSIVE GENOMIC PROFILING (CGP) OF METASTATIC INVASIVE LOBULAR CARCINOMAS REVEALS HETEROGENEITY

(71) Applicant: Foundation Medicine, Inc., Cambridge, MA (US)

(72) Inventor: Ethan SOKOL, Somerville, MA (US)

(73) Assignee: Foundation Medicine, Inc., Cambridge, MA (US)

(21) Appl. No.: 18/037,309

(22) PCT Filed: Dec. 6, 2021
(86) PCT No.: PCT/US2021/062070

§ 371 Date: May 16, 2023

Related U.S. Application Data
(60) Provisional application No. 63/194,847, filed on May 28, 2021.
(60) Provisional application No. 63/122,431, filed on Dec. 7, 2020.

(51) Int. Cl. C12Q 1/6886; C07K 16/28; G16B 20/20; G16B 30/00; G16B 40/00

(52) U.S. Cl. CPC C12Q 1/6886; C07K 16/2827; G16B 20/20; G16B 30/00; G16B 40/00; C12Q 2600/156; C12Q 2600/158; C12Q 2600/106

(57) ABSTRACT

The present disclosure relates to methods of treating or delaying progression of invasive lobular carcinoma (ILC) metastasis in an individual, methods for evaluating, identifying, and/or assessing an individual having an ILC metastasis, as well as methods for genomic profiling of an ILC metastasis.

### CROSS-REFERENCE TO RELATED APPLICATIONS

This application claims the benefit of U.S. Provisional Application No. 63/194,847, filed May 28, 2021, and U.S. Provisional Application No. 63/122,431, filed Dec. 7, 2020, the contents of each of which are hereby incorporated by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 197102005540SEQLIST.TXT, date recorded: Dec. 6, 2021, size: 518,690 bytes).

### FIELD OF THE INVENTION

The present disclosure relates to methods of treating metastatic invasive lobular carcinoma, and to methods for genomic profiling of metastatic invasive lobular carcinoma.

### BACKGROUND OF THE INVENTION

Metastatic breast cancer is a clinically challenging disease with poor outcomes. Invasive lobular carcinoma (ILC) is a rare subtype of breast cancer with distinct patterns of metastasis, including frequent gastrointestinal and female reproductive system metastases. Metastatic ILC is particularly challenging since patients frequently exhibit late relapse and have poorer prognoses than the stage and/or grade of their disease would suggest, for example, relative to invasive ductal carcinomas (IDC).

Due to the relative rarity of ILC and the lack of large comprehensive genomic profiling (CGP) datasets in metastatic ILC, the genomic landscape of metastatic ILC has not been systematically explored, including differences in the genomic landscape among different ILC metastatic sites.

Accordingly, there is a need in the art for characterizing the genomic landscape of metastatic ILC to identify genetic lesions associated with such cancers, and for developing methods of identifying, evaluating, and treating patients with metastatic ILC having such genetic lesions.

All references cited herein, including patents, patent applications and publications, are hereby incorporated by reference in their entirety. To the extent that any reference incorporated by reference conflicts with the instant disclosure, the instant disclosure shall control.

### BRIEF SUMMARY OF THE INVENTION

In one aspect, provided herein is a method of treating or delaying progression of invasive lobular carcinoma (ILC) metastasis, comprising: (a) acquiring knowledge of a tumor mutational burden (TMB) of at least about 10 mutations/megabase (mut/Mb) in a sample from an individual having an ILC metastasis; and (b) responsive to said knowledge, administering to the individual an effective amount of an immune checkpoint inhibitor. In some embodiments, the acquiring knowledge comprises measuring the level of TMB in a sample obtained from the individual. In some embodiments, TMB is measured on between about 0.8 Mb and about 1.1 Mb. In some embodiments, the individual is a human. In some embodiments, the ILC metastasis is selected from a bone ILC metastasis, a female reproductive system ILC metastasis, a gastrointestinal ILC metastasis, a liver ILC metastasis, or a skin ILC metastasis. In some embodiments, the ILC metastasis is a bone ILC metastasis. In some embodiments, the ILC metastasis is a female reproductive system ILC metastasis. In some embodiments, the ILC metastasis is a gastrointestinal ILC metastasis. In some embodiments, the ILC metastasis is a liver ILC metastasis. In some embodiments, the ILC metastasis is a skin ILC metastasis. In some embodiments, the sample from the individual comprises fluid, cells, or tissue. In some embodiments, the sample from the individual comprises a tumor biopsy or a circulating tumor cell. In some embodiments, the sample from the individual comprises nucleic acids. In some embodiments, the sample from the individual comprises mRNA, genomic DNA, circulating tumor DNA, cell-free DNA, or cell-free RNA. In some embodiments, TMB is measured in the sample by whole exome sequencing, whole genome sequencing, or gene-targeted sequencing. In some embodiments, the immune checkpoint inhibitor is a small molecule inhibitor, an antibody or antibody fragment, a peptide, a fusion protein, or a nucleic acid. In some embodiments, the immune checkpoint inhibitor is a PD-1 binding antagonist or a PD-L1 binding antagonist. In some embodiments, the immune checkpoint inhibitor is an anti-PD-1 antibody or antibody fragment. In some embodiments, the anti-PD-1 antibody or antibody fragment is selected from MDX-1106 (nivolumab), MK-3475 (pembrolizumab), MEDI-0680 (AMP-514), PDR001, REGN2810, MGA-012, JNJ-63723283, BI 754091, BGB-108, BGB-A317, JS-001, STI-A1110, INCSHR-1210, PF-06801591, TSR-042, AM0001, ENUM 244C8, or ENUM 388D4. In some embodiments, the anti-PD-1 antibody is pembrolizumab. In some embodiments, the immune checkpoint inhibitor is an anti-PD-L1 antibody or antibody fragment. In some embodiments, the anti-PD-L1 antibody or antibody fragment is selected from YW243.55.S70, MPDL3280A (atezolizumab), MDX-1105, MEDI4736 (durvalumab), MSB0010718C (avelumab), LY3300054, STI-A1014, KN035, FAZ053, or CX-072. In some embodiments, the method further comprises administering an additional anti-cancer therapy to the individual. In some embodiments, the additional anti-cancer therapy is a surgery, a radiotherapy, a chemotherapy, an anti-angiogenic therapy, an anti-DNA repair therapy, an immunotherapy, an anti-neoplastic agent, a cytotoxic agent, an anti-inflammatory therapy, or any combination thereof. In some embodiments, the ILC metastasis comprises one or more deleterious CDH1 mutations.

In another aspect, provided herein is a method of treating or delaying progression of invasive lobular carcinoma (ILC) metastasis, comprising: (a) acquiring knowledge of a PD-L1-positive ILC metastasis in a sample from an individual having an ILC metastasis; and (b) responsive to said knowledge, administering to the individual an effective amount of an immune checkpoint inhibitor. In some embodiments, the acquiring knowledge of a PD-L1-positive ILC metastasis comprises measuring the level of PD-L1 expression in a sample obtained from the individual. In some embodiments, the level of PD-L1 expression is measured using an immunohistochemistry assay. In some embodiments, the level of PD-L1 expression is determined based on PD-L1 expression in tumor infiltrating immune cells (ICs) and/or tumor cells (TCs). In some embodiments, the acquiring knowledge of a PD-L1-positive ILC metastasis comprises acquiring knowledge that at least about 1% of ICs in the sample are PD-L1-positive. In some embodiments, the individual is a human. In some embodiments, the ILC metastasis is selected from a bone ILC metastasis, a female reproductive system ILC metastasis, a gastrointestinal ILC metastasis, a liver ILC metastasis, or a skin ILC metastasis. In some embodiments, the ILC metastasis is a bone ILC metastasis. In some embodiments, the ILC metastasis is a female reproductive system ILC metastasis. In some embodiments, the ILC metastasis is a gastrointestinal ILC metastasis. In some embodiments, the ILC metastasis is a liver ILC metastasis. In some embodiments, the ILC metastasis is a skin ILC metastasis. In some embodiments, the sample from the individual comprises fluid, cells, or tissue. In some embodiments, the sample from the individual comprises a tumor biopsy or a circulating tumor cell. In some embodiments, the immune checkpoint inhibitor is a small molecule inhibitor, an antibody or antibody fragment, a peptide, a fusion protein, or a nucleic acid. In some embodiments, the immune checkpoint inhibitor is a PD-1 binding antagonist or a PD-L1 binding antagonist. In some embodiments, the immune checkpoint inhibitor is an anti-PD-1 antibody or antibody fragment. In some embodiments, the anti-PD-1 antibody or antibody fragment is selected from MDX-1106 (nivolumab), MK-3475 (pembrolizumab), MEDI-0680 (AMP-514), PDR001, REGN2810, MGA-012, JNJ-63723283, BI 754091, BGB-108, BGB-A317, JS-001, STI-A1110, INCSHR-1210, PF-06801591, TSR-042, AM0001, ENUM 244C8, or ENUM 388D4. In some embodiments, the anti-PD-1 antibody is pembrolizumab. In some embodiments, the immune checkpoint inhibitor is an anti-PD-L1 antibody or antibody fragment. In some embodiments, the anti-PD-L1 antibody or antibody fragment is selected from YW243.55.S70, MPDL3280A (atezolizumab), MDX-1105, MEDI4736 (durvalumab), MSB0010718C (avelumab), LY3300054, STI-A1014, KN035, FAZ053, or CX-072. In some embodiments, the method further comprises administering an additional anti-cancer therapy to the individual. In some embodiments, the additional anti-cancer therapy is a surgery, a radiotherapy, a chemotherapy, an anti-angiogenic therapy, an anti-DNA repair therapy, an immunotherapy, an anti-neoplastic agent, a cytotoxic agent, an anti-inflammatory therapy, or any combination thereof. In some embodiments, the ILC metastasis comprises one or more deleterious CDH1 mutations.

In another aspect, provided herein is a method of treating or delaying progression of invasive lobular carcinoma (ILC) metastasis, comprising: (a) acquiring knowledge of an alteration in one or more genes in a sample from an individual having an ILC metastasis, wherein the one or more genes are selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D; and (b) responsive to said knowledge, administering to the individual an effective amount of an anti-cancer agent. In some embodiments, the acquiring knowledge comprises detecting the alteration in the one or more genes in a sample obtained from the individual. In some embodiments, the individual is a human. In some embodiments, the ILC metastasis is selected from a bone ILC metastasis, a female reproductive system ILC metastasis, a gastrointestinal ILC metastasis, a liver ILC metastasis, or a skin ILC metastasis. In some embodiments, the ILC metastasis is a bone ILC metastasis. In some embodiments, the ILC metastasis is a female reproductive system ILC metastasis. In some embodiments, the ILC metastasis is a gastrointestinal ILC metastasis. In some embodiments, the ILC metastasis is a liver ILC metastasis. In some embodiments, the ILC metastasis is a skin ILC metastasis. In some embodiments, the anti-cancer agent is a small molecule, a chemotherapy, an antibody or antibody fragment, a cellular immunotherapy, an immune checkpoint inhibitor, or a nucleic acid.

In some embodiments, the alteration is an alteration in PIK3CA. In some embodiments, the alteration comprises one or more of a substitution of one or more nucleotides, an insertion of one or more nucleotides, or a deletion of one or more nucleotides. In some embodiments, the alteration results in one or more amino acid substitutions in a polypeptide encoded by the PIK3CA gene at amino acid position E81, R108, K111, G118, N345, D350, E365, E418, C420, E453, P539, E542, E545, Q546, E726, E970, M1004, M1043, N1044, H1047, G1049, or any combination thereof, wherein the numbering of the residues is according to the amino acid sequence of SEQ ID NO: 26. In some embodiments, the one or more amino acid substitutions comprise a E81K, R108H, K111N, G118D, N345K, D350N, E365K, E418K, C420R, E453K, E453Q, P539R, E542K, E545K, E545A, E545Q, Q546R, Q546K, E726K, E970K, M1004I, M1043I, N1044K, H1047R, H1047L, or G1049R amino acid substitution, or any combination thereof, wherein the numbering of the residues is according to the amino acid sequence of SEQ ID NO: 26. In some embodiments, the alteration results in a deletion of amino acid residue E110 (E110del) in a polypeptide encoded by the PIK3CA gene, wherein the numbering of the residues is according to the amino acid sequence of SEQ ID NO: 26. In some embodiments, the anti-cancer agent is a kinase inhibitor, an AKT inhibitor, an EZH2 inhibitor, or an mTOR inhibitor. In some embodiments, the ILC metastasis is a skin ILC metastasis. In some embodiments, the ILC metastasis is a gastrointestinal ILC metastasis. In some embodiments, the ILC metastasis is a liver ILC metastasis. In some embodiments, the ILC metastasis is a female reproductive system ILC metastasis. In some embodiments, the ILC metastasis is a bone ILC

In some embodiments, the alteration is an alteration in BRCA1 or BRCA2. In some embodiments, the anti-cancer agent is a PARP inhibitor, a Chk1/2 inhibitor, or a Wee1 inhibitor. In some embodiments, the ILC metastasis is a female reproductive system ILC metastasis. In some embodiments, the ILC metastasis is a bone ILC metastasis. In some embodiments, the ILC metastasis is a skin ILC metastasis. In some embodiments, the ILC metastasis is a liver ILC metastasis.

In some embodiments, the alteration is an alteration in ESR1. In some embodiments, the alteration comprises one or more of a substitution of one or more nucleotides, an insertion of one or more nucleotides, or a deletion of one or more nucleotides. In some embodiments, the alteration results in one or more amino acid substitutions in a polypeptide encoded by the ESR1 gene at amino acid position E380, V418, S463, V533, L536, Y537, D538, or any combination thereof, wherein the numbering of the residues is according to the amino acid sequence of SEQ ID NO: 29. In some embodiments, the one or more amino acid substitutions comprise a E380Q, V418E, S463P, V533M, L536Q, L536H, L536P, L536R, Y537S, Y537N, Y537C, Y537D, or D538G amino acid substitution, or any combination thereof, wherein the numbering of the residues is according to the amino acid sequence of SEQ ID NO: 29. In some embodiments, the alteration results in a deletion of amino acid V422 (V422del) and/or a deletion of amino acids V533-L536 (V533_L536del) in a polypeptide encoded by the ESR1 gene, wherein the numbering of the residues is according to the amino acid sequence of SEQ ID NO: 29. In some embodiments, the anti-cancer agent is a Selective Estrogen Receptor Modulator (SERM), an anti-estrogen, or an aromatase inhibitor. In some embodiments, the ILC metastasis is a gastrointestinal ILC metastasis. In some embodiments, the ILC metastasis is a liver ILC metastasis. In some embodiments, the ILC metastasis is a bone ILC metastasis. In some embodiments, the ILC metastasis is a skin ILC metastasis.

In some embodiments, the alteration is an alteration in NF1. In some embodiments, the alteration comprises one or more of a substitution of one or more nucleotides, an insertion of one or more nucleotides, or a deletion of one or more nucleotides. In some embodiments, the anti-cancer agent is a kinase inhibitor, an mTOR inhibitor, an EGFR inhibitor, a glutaminase inhibitor, or a MEK inhibitor. In some embodiments, the ILC metastasis is a gastrointestinal ILC metastasis. In some embodiments, the ILC metastasis is a liver ILC metastasis. In some embodiments, the ILC metastasis is a female reproductive system ILC metastasis. In some embodiments, the ILC metastasis is a bone ILC metastasis. In some embodiments, the ILC metastasis is a skin ILC metastasis.

In some embodiments, the alteration is an alteration in RB1. In some embodiments, the alteration comprises one or more of a substitution of one or more nucleotides, an insertion of one or more nucleotides, or a deletion of one or more nucleotides. In some embodiments, the anti-cancer agent is a SOX2 inhibitor, an EZH2 inhibitor, a chemotherapy, a checkpoint kinase (CHK) inhibitor, a CDC25 phosphatase inhibitor, a polo-like kinase (PLK) inhibitor, or an aurora kinase (AURK) inhibitor. In some embodiments, the ILC metastasis is a gastrointestinal ILC metastasis. In some embodiments, the ILC metastasis is a liver ILC metastasis. In some embodiments, the ILC metastasis is a skin ILC metastasis. In some embodiments, the ILC metastasis is a bone ILC metastasis.

In some embodiments, the alteration is an alteration in KRAS. In some embodiments, the alteration comprises one or more of a substitution of one or more nucleotides, an insertion of one or more nucleotides, or a deletion of one or more nucleotides. In some embodiments, the alteration results in one or more amino acid substitutions in a polypeptide encoded by the KRAS gene at amino acid position G12, G13, L19, Q61, A146, K147, F156, or any combination thereof, wherein the numbering of the residues is according to the amino acid sequence of SEQ ID NO: 33. In some embodiments, the one or more amino acid substitutions comprise a G12V, G12D, G12A, G12R, G12S, G12C, G12L, G13D, L19F, Q61H, Q61K, Q61E, A146T, K147N, or F156L amino acid substitution, or any combination thereof, wherein the numbering of the residues is according to the amino acid sequence of SEQ ID NO: 33. In some embodiments, the anti-cancer agent is a kinase inhibitor, a farnesyltransferase inhibitor, a geranylgeranyltransferase inhibitor, a palmitoylation inhibitor, an inhibitor of methylation cleavage, a Raf inhibitor, a MEK inhibitor, an mTOR inhibitor, or an agent that inhibits the modification or post-translational processing of KRAS. In some embodiments, the ILC metastasis is a gastrointestinal ILC metastasis. In some embodiments, the ILC metastasis is a liver ILC metastasis. In some embodiments, the ILC metastasis is a bone ILC metastasis.

In some embodiments, the alteration is an alteration in ERBB2. In some embodiments, the alteration comprises one or more of a substitution of one or more nucleotides, an insertion of one or more nucleotides, or a deletion of one or more nucleotides. In some embodiments, the alteration results in one or more amino acid substitutions in a polypeptide encoded by the ERBB2 gene at amino acid position S310, S653, V659, R678, V697, E717, T733, L755, I767, D769, G776, V777, T798, V842, L869, or any combination thereof, wherein the numbering of the residues is according to the amino acid sequence of SEQ ID NO: 34. In some embodiments, the one or more amino acid substitutions comprise a S310F, S310Y, S653C, V659D, R678Q, V697L, E717K, T733I, L755S, L755P, I767M, D769N, D769Y, D769H, G776V, V777L, T798I, V842I, or L869R amino acid substitution, or any combination thereof, wherein the numbering of the residues is according to the amino acid sequence of SEQ ID NO: 34. In some embodiments, the alteration results in a deletion of amino acids L755-T759 (L755_T759del) and/or amino acids L755-E757 (L755_E757del) in a polypeptide encoded by the ERBB2 gene, wherein the numbering of the residues is according to the amino acid sequence of SEQ ID NO: 34. In some embodiments, the alteration results in an insertion of one or more amino acid residues between amino acid residues A775 and G776, and/or between amino acid residues P780 and Y781 in a polypeptide encoded by the ERBB2 gene, wherein the numbering of the residues is according to the amino acid sequence of SEQ ID NO: 34. In some embodiments, the alteration results in an insertion of the amino acid sequence YVMA (SEQ ID NO: 51) between amino acid residues A775 and G776 (A775_G776insYVMA), and/or of the amino acid sequence GSP (SEQ ID NO: 52) between amino acid residues P780 and Y781 (P780_Y781insGSP), in a polypeptide encoded by the ERBB2 gene, wherein the numbering of the residues is according to the amino acid sequence of SEQ ID NO: 34. In some embodiments, the anti-cancer agent is a kinase inhibitor, a small molecule, an antibody or antibody fragment, a cellular immunotherapy, or a pan-ERBB inhibitor. In some embodiments, the kinase inhibitor is a multi-specific kinase inhibitor, a reversible HER2 inhibitor, an irreversible HER2 inhibitor, a pan-ERBB inhibitor, a dual HER2 inhibitor, a HER2-specific inhibitor, an EGFR inhibitor, or a dual EGFR/ERBB inhibitor. In some embodiments, the ILC metastasis is a liver ILC metastasis. In some embodiments, the ILC metastasis is a bone ILC metastasis. In some embodiments, the ILC metastasis is a skin ILC metastasis.

In some embodiments, the alteration is an alteration in BRAF. In some embodiments, the alteration comprises one or more of a substitution of one or more nucleotides, an insertion of one or more nucleotides, or a deletion of one or more nucleotides. In some embodiments, the alteration results in one or more amino acid substitutions in a polypeptide encoded by the BRAF gene at amino acid position D380, G464, G466, S467, G469, L485, L584, E586, D594, V600, or any combination thereof, wherein the numbering of the residues is according to the amino acid sequence of SEQ ID NO: 35. In some embodiments, the one or more amino acid substitutions comprise a D380H, G464R, G466E, S467L, G469A, G469E, G469R, L485F, L584F, E586K, D594N, D594G, or V600E amino acid substitution, or any combination thereof, wherein the numbering of the residues is according to the amino acid sequence of SEQ ID NO: 35. In some embodiments, the anti-cancer agent is a kinase inhibitor. In some embodiments, the ILC metastasis is a bone ILC metastasis.

In some embodiments, the alteration is an alteration in ARID1A. In some embodiments, the alteration comprises one or more of a substitution of one or more nucleotides, an insertion of one or more nucleotides, or a deletion of one or more nucleotides. In some embodiments, the anti-cancer agent is a PARP inhibitor, a bromodomain-containing protein 4 (BRD4) inhibitor, an HDAC inhibitor, a PI3K inhibitor, an ATR inhibitor, an EZH2 inhibitor, an AKT inhibitor, a TrxR inhibitor, a GSH inhibitor, or an immune checkpoint inhibitor. In some embodiments, the ILC metastasis is a gastrointestinal ILC metastasis. In some embodiments, the ILC metastasis is a liver ILC metastasis. In some embodiments, the ILC metastasis is a female reproductive system ILC metastasis. In some embodiments, the ILC metastasis is a bone ILC metastasis. In some embodiments, the ILC metastasis is a skin ILC metastasis.

In some embodiments, the alteration is an alteration in PTEN. In some embodiments, the alteration is a PTEN deletion. In some embodiments, the anti-cancer agent is a PI3K inhibitor, an AKT inhibitor, an mTOR inhibitor, or a MET inhibitor. In some embodiments, the ILC metastasis is a skin ILC metastasis. In some embodiments, the ILC metastasis is a liver ILC metastasis. In some embodiments, the ILC metastasis is a female reproductive system ILC metastasis.

In some embodiments, the alteration is an alteration in FGFR2. In some embodiments, the alteration comprises one or more of a substitution of one or more nucleotides, an insertion of one or more nucleotides, or a deletion of one or more nucleotides. In some embodiments, the alteration results in one or more amino acid substitutions in a polypeptide encoded by the FGFR2 gene at amino acid position S252, P253, Y375, C382, M391, V395, M537, N549, K659, R664, or any combination thereof, wherein the numbering of the residues is according to the amino acid sequence of SEQ ID NO: 38. In some embodiments, the one or more amino acid substitutions comprise a S252W, P253R, Y375C, C382R, M391R, V395D, M537I, N549D, N549K, K659M, K659E, K659N, or R664W amino acid substitution, or any combination thereof, wherein the numbering of the residues is according to the amino acid sequence of SEQ ID NO: 38. In some embodiments, the alteration results in a frameshift of S799fs*22, wherein the numbering of the residues is according to the amino acid sequence of SEQ ID NO: 38. In some embodiments, the anti-cancer agent is a kinase inhibitor. In some embodiments, the ILC metastasis is a liver ILC metastasis. In some embodiments, the ILC metastasis is a bone ILC metastasis.

In some embodiments, the alteration is an alteration in SMAD4. In some embodiments, the anti-cancer agent is a PARP inhibitor. In some embodiments, the ILC metastasis is a liver ILC metastasis.

In some embodiments, the alteration is an alteration in PTPN11. In some embodiments, the alteration comprises one or more of a substitution of one or more nucleotides, an insertion of one or more nucleotides, or a deletion of one or more nucleotides. In some embodiments, the anti-cancer agent is a small molecule inhibitor or a kinase inhibitor.

In some embodiments, the alteration is an alteration in TERT. In some embodiments, the alteration comprises one or more of a substitution of one or more nucleotides, an insertion of one or more nucleotides, or a deletion of one or more nucleotides. In some embodiments, the alteration is an alteration in the promoter of TERT, wherein the alteration in the promoter of TERT comprises a −146C>T, −139_−138CC>TT, or −124C>T mutation, or any combination thereof, wherein the numbering of the nucleotides is according to SEQ ID NO: 22. In some embodiments, the anti-cancer agent is a small molecule inhibitor, a modified nucleotide or nucleoside analog, a G-quadruplex stabilizer, a heat shock protein (HSP) inhibitor, or a MYC inhibitor. In some embodiments, the ILC metastasis is a gastrointestinal ILC metastasis.

In some embodiments, the alteration is an alteration in ALK. In some embodiments, the alteration comprises one or more of a substitution of one or more nucleotides, an insertion of one or more nucleotides, or a deletion of one or more nucleotides. In some embodiments, the anti-cancer agent is a kinase inhibitor, a heat shock protein (HSP) inhibitor or a MYC inhibitor.

In some embodiments, the alteration is an alteration in NCOR1. In some embodiments, the alteration comprises one or more of a substitution of one or more nucleotides, an insertion of one or more nucleotides, or a deletion of one or more nucleotides. In some embodiments, the ILC metastasis is a female reproductive system ILC metastasis.

In some embodiments, the alteration is an alteration in APC. In some embodiments, the alteration comprises one or more of a substitution of one or more nucleotides, an insertion of one or more nucleotides, or a deletion of one or more nucleotides. In some embodiments, the anti-cancer agent is a beta-catenin inhibitor or an APC inhibitor.

In some embodiments, which may be combined with any of the preceding embodiments, the method further comprises administering an additional anti-cancer therapy to the individual. In some embodiments, the additional anti-cancer therapy is a surgery, a radiotherapy, a chemotherapy, an anti-angiogenic therapy, an anti-DNA repair therapy, an immunotherapy, an anti-neoplastic agent, a cytotoxic agent, an anti-inflammatory therapy, or any combination thereof. In some embodiments, the ILC metastasis comprises one or more deleterious CDH1 mutations. In some embodiments, the method further comprises acquiring knowledge of one or more deleterious CDH1 mutations in a sample from the individual. In some embodiments, the acquiring knowledge comprises detecting the one or more deleterious CDH1 mutations in a sample from the individual. In some embodiments, the one or more deleterious CDH1 mutations comprise one or more of a substitution of one or more nucleotides, an insertion of one or more nucleotides, or a deletion of one or more nucleotides in a gene encoding a CDH1 polypeptide. In some embodiments, the one or more deleterious CDH1 mutations result in loss of function of a CDH1 polypeptide. In some embodiments, the one or more deleterious CDH1 mutations result in a mutation of the CDH1 start codon. In some embodiments, the one or more deleterious CDH1 mutations result in one or more amino acid substitutions in a CDH1 polypeptide at amino acid position E243, D402, D433, A634, or any combination thereof, wherein the numbering of the residues is according to the amino acid sequence of SEQ ID NO: 50. In some embodiments, the one or more amino acid substitutions comprise a E243K, D402N, D433N, or A634V amino acid substitution, or any combination thereof, wherein the numbering of the residues is according to the amino acid sequence of SEQ ID NO: 50. In some embodiments, the one or more deleterious CDH1 mutations result in a stop codon at amino acid position W4 (W4*), S9 (S9*), Q16 (Q16*), W20 (W20*), Q23 (Q23*), E24 (E24*), E26 (E26*), E35 (E35*), Y37 (Y37*), E47 (E47*), R54 (R54*), E58 (E58*), R63 (R63*), Q64 (Q64*), R74 (R74*), W103 (W103*), Q129 (Q129*), R150 (R150*), Q152 (Q152*), Q177 (Q177*), Y190 (Y190*), Y228 (Y228*), E243 (E243*), Q255 (Q255*), Q264 (Q264*), Y302 (Y302*), R335 (R335*), Q346 (Q346*), E353 (E353*), Q383 (Q383*), Q449 (Q449*), E463 (E463*), Y523 (Y523*), W526 (W526*), R598 (R598*), Q610 (Q610*), Q641 (Q641*), E648 (E648*), Q699 (Q699*), Q706 (Q706*), Q765 (Q765*), Q771 (Q771*), E806 (E806*), Y827 (Y827*), or any combination thereof, wherein the numbering of the residues is according to the amino acid sequence of SEQ ID NO: 50. In some embodiments, the one or more deleterious CDH1 mutations comprise a frameshift, wherein the frameshift is an T115fs*53, P127fs*41, P200fs*6, P200fs*16, V202fs*7, H233fs*11, P277fs*5, Y302fs*1, S337fs*12, Y523fs*1, L585fs*4, I650fs*3, I650fs*13, A719fs*29, or Q765fs*4 frameshift, wherein the numbering of the residues is according to the amino acid sequence of SEQ ID NO: 50. In some embodiments, the one or more deleterious CDH1 mutations comprise a splice site mutation, wherein the splice site mutation is a 48+1G>A, 1565+1G>A, or 1565+1G>T splice site mutation, wherein the numbering of the nucleotides is according to SEQ ID NO: 49. In some embodiments, the sample from the individual comprises fluid, cells, or tissue. In some embodiments, the sample from the individual comprises a tumor biopsy or a circulating tumor cell. In some embodiments, the sample from the individual comprises nucleic acids. In some embodiments, the sample from the individual comprises mRNA, genomic DNA, circulating tumor DNA, cell-free DNA, or cell-free RNA. In some embodiments, the alteration is detected in the sample by one or more methods selected from a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping. In some embodiments, the sample from the individual comprises one or more proteins. In some embodiments, the acquiring knowledge comprises detecting the alteration in a polypeptide encoded by the one or more genes in the sample from the individual. In some embodiments, the alteration is detected in the sample by one or more methods selected from immunoblotting, enzyme linked immunosorbent assay (ELISA), immunohistochemistry, or mass spectrometry.

In another aspect, provided herein is a method for genomic profiling of an invasive lobular carcinoma (ILC) metastasis, comprising: (a) detecting one or more biomarkers in a sample from an individual having an ILC metastasis, wherein the one or more biomarkers are selected from: (i) a tumor mutational burden (TMB) of at least about 10 mutations/megabase (mut/Mb), (ii) a PD-L1-positive ILC metastasis, or (iii) an alteration in one or more genes, wherein the one or more genes are selected from: PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D; and (b) providing a report to a party. In some embodiments, the individual is a human. In some embodiments, the ILC metastasis is selected from a bone ILC metastasis, a female reproductive system ILC metastasis, a gastrointestinal ILC metastasis, a liver ILC metastasis, or a skin ILC metastasis. In some embodiments, the ILC metastasis is a bone ILC metastasis. In some embodiments, the ILC metastasis is a female reproductive system ILC metastasis. In some embodiments, the ILC metastasis is a gastrointestinal ILC metastasis. In some embodiments, the ILC metastasis is a liver ILC metastasis. In some embodiments, the ILC metastasis is a skin ILC metastasis.

In some embodiments, the ILC metastasis comprises one or more deleterious CDH1 mutations. In some embodiments, the method further comprises acquiring knowledge of one or more deleterious CDH1 mutations in a sample from the individual. In some embodiments, the acquiring knowledge comprises detecting the one or more deleterious CDH1 mutations in a sample from the individual. In some embodiments, the one or more deleterious CDH1 mutations comprise one or more of a substitution of one or more nucleotides, an insertion of one or more nucleotides, or a deletion of one or more nucleotides in a gene encoding a CDH1 polypeptide. In some embodiments, the one or more deleterious CDH1 mutations result in loss of function of a CDH1 polypeptide. In some embodiments, the one or more deleterious CDH1 mutations result in a mutation of the CDH1 start codon. In some embodiments, the one or more deleterious CDH1 mutations result in one or more amino acid substitutions in a CDH1 polypeptide at amino acid position E243, D402, D433, A634, or any combination thereof, wherein the numbering of the residues is according to the amino acid sequence of SEQ ID NO: 50. In some embodiments, the one or more amino acid substitutions comprise a E243K, D402N, D433N, or A634V amino acid substitution, or any combination thereof, wherein the numbering of the residues is according to the amino acid sequence of SEQ ID NO: 50. In some embodiments, the one or more deleterious CDH1 mutations result in a stop codon at amino acid position W4 (W4*), S9 (S9*), Q16 (Q16*), W20 (W20*), Q23 (Q23*), E24 (E24*), E26 (E26*), E35 (E35*), Y37 (Y37*), E47 (E47*), R54 (R54*), E58 (E58*), R63 (R63*), Q64 (Q64*), R74 (R74*), W103 (W103*), Q129 (Q129*), R150 (R150*), Q152 (Q152*), Q177 (Q177*), Y190 (Y190*), Y228 (Y228*), E243 (E243*), Q255 (Q255*), Q264 (Q264*), Y302 (Y302*), R335 (R335*), Q346 (Q346*), E353 (E353*), Q383 (Q383*), Q449 (Q449*), E463 (E463*), Y523 (Y523*), W526 (W526*), R598 (R598*), Q610 (Q610*), Q641 (Q641*), E648 (E648*), Q699 (Q699*), Q706 (Q706*), Q765 (Q765*), Q771 (Q771*), E806 (E806*), Y827 (Y827*), or any combination thereof, wherein the numbering of the residues is according to the amino acid sequence of SEQ ID NO: 50. In some embodiments, the one or more deleterious CDH1 mutations comprise a frameshift, wherein the frameshift is a T115fs*53, P127fs*41, P200fs*6, P200fs*16, V202fs*7, H233fs*11, P277fs*5, Y302fs*1, S337fs*12, Y523fs*1, L585fs*4, I650fs*3, I650fs*13, A719fs*29, or Q765fs*4 frameshift, wherein the numbering of the residues is according to the amino acid sequence of SEQ ID NO: 50. In some embodiments, the one or more deleterious CDH1 mutations comprise a splice site mutation, wherein the splice site mutation is a 48+1G>A, 1565+1G>A, or 1565+1G>T splice site mutation, wherein the numbering of the nucleotides is according to SEQ ID NO: 49. In some embodiments, the sample from the individual comprises fluid, cells, or tissue. In some embodiments, the sample from the individual comprises a tumor biopsy or a circulating tumor cell. In some embodiments, the sample from the individual comprises nucleic acids. In some embodiments, the sample from the individual comprises mRNA, genomic DNA, circulating tumor DNA, cell-free DNA, or cell-free RNA.

In some embodiments, detecting a TMB of at least about 10 mut/Mb comprises measuring the level of TMB in the sample from the individual. In some embodiments, TMB is measured on between about 0.8 Mb and about 1.1 Mb. In some embodiments, TMB is measured in the sample by whole exome sequencing, whole genome sequencing, or gene-targeted sequencing. In some embodiments, the method comprises detecting a tumor mutational burden (TMB) of at least about 10 mutations/megabase (mut/Mb) in a sample from an individual having a gastrointestinal ILC metastasis or a skin ILC metastasis. In some embodiments, the method comprises detecting a TMB of at least about 10 mut/Mb in a sample from an individual having a gastrointestinal ILC metastasis. In some embodiments, the method comprises detecting a TMB of at least about 10 mut/Mb in a sample from an individual having a liver ILC metastasis. In some embodiments, the method comprises detecting a TMB of at least about 10 mut/Mb in a sample from an individual having a female reproductive system ILC metastasis. In some embodiments, the method comprises detecting a TMB of at least about 10 mut/Mb in a sample from an individual having a bone ILC metastasis. In some embodiments, the method comprises detecting a TMB of at least about 10 mut/Mb in a sample from an individual having a skin ILC metastasis.

In some embodiments, detecting a PD-L1-positive ILC metastasis comprises measuring the level of PD-L1 expression in the sample from the individual. In some embodiments, the level of PD-LL expression is measured using an immunohistochemistry assay. In some embodiments, the level of PD-L1 expression is determined based on PD-L1 expression in tumor infiltrating immune cells (ICs) and/or tumor cells (TCs). In some embodiments, a PD-L1-positive ILC metastasis is detected if at least about 1% of ICs in the sample are PD-L1-positive.

In some embodiments, the method comprises detecting a PD-L1-positive ILC metastasis in a sample from an individual having a gastrointestinal ILC metastasis. In some embodiments, the method comprises detecting a PD-L1-positive ILC metastasis in a sample from an individual having a liver ILC metastasis. In some embodiments, the method comprises detecting a PD-L1-positive ILC metastasis in a sample from an individual having a female reproductive system ILC metastasis. In some embodiments, the method comprises detecting a PD-L1-positive ILC metastasis in a sample from an individual having a skin ILC metastasis.

In some embodiments, the method comprises detecting: (a) one or more of a substitution of one or more nucleotides, an insertion of one or more nucleotides, or a deletion of one or more nucleotides in one or more genes selected from PIK3CA, ESR1, NF1, RB1, ERBB2, ARID1A, NCOR1, FOXP1, APC, CASP8, PTPN11, TERT, FGFR2, KRAS, BRAF, SMAD4, SOX9 or ALK; (b) a PTEN deletion; or (c) a KMT2D rearrangement.

In some embodiments, the method comprises detecting an alteration in BRCA1 and/or BRCA2 in a sample from an individual having a female reproductive system ILC metastasis. In some embodiments, the method comprises detecting an alteration in BRCA1 and/or BRCA2 in a sample from an individual having a bone ILC metastasis. In some embodiments, the method comprises detecting an alteration in BRCA1 and/or BRCA2 in a sample from an individual having a skin ILC metastasis. In some embodiments, the method comprises detecting an alteration in BRCA1 and/or BRCA2 in a sample from an individual having a liver ILC metastasis.

In some embodiments, the method comprises detecting an alteration in PIK3CA in a sample from an individual having a gastrointestinal ILC metastasis. In some embodiments, the method comprises detecting an alteration in PIK3CA in a sample from an individual having a liver ILC metastasis. In some embodiments, the method comprises detecting an alteration in PIK3CA in a sample from an individual having a female reproductive system ILC metastasis. In some embodiments, the method comprises detecting an alteration in PIK3CA in a sample from an individual having a bone ILC metastasis. In some embodiments, the method comprises detecting an alteration in PIK3CA in a sample from an individual having a skin ILC metastasis. In some embodiments, the alteration results in one or more amino acid substitutions in a polypeptide encoded by the PIK3CA gene at amino acid position E81, R108, K111, G118, N345, D350, E365, E418, C420, E453, P539, E542, E545, Q546, E726, E970, M1004, M1043, N1044, H1047, G1049, or any combination thereof, wherein the numbering of the residues is according to the amino acid sequence of SEQ ID NO: 26. In some embodiments, the one or more amino acid substitutions comprise a E81K, R108H, K111N, G118D, N345K, D350N, E365K, E418K, C420R, E453K, E453Q, P539R, E542K, E545K, E545A, E545Q, Q546R, Q546K, E726K, E970K, M1004I, M1043I, N1044K, H1047R, H1047L, or G1049R amino acid substitution, or any combination thereof, wherein the numbering of the residues is according to the amino acid sequence of SEQ ID NO: 26. In some embodiments, the alteration results in a deletion of amino acid residue E110 (E110del) in a polypeptide encoded by the PIK3CA gene, wherein the numbering of the residues is according to the amino acid sequence of SEQ ID NO: 26.

In some embodiments, the method comprises detecting an alteration in ESR1 in a sample from an individual having a gastrointestinal ILC metastasis. In some embodiments, the method comprises detecting an alteration in ESR1 in a sample from an individual having a liver ILC metastasis. In some embodiments, the method comprises detecting an alteration in ESR1 in a sample from an individual having a bone ILC metastasis. In some embodiments, the method comprises detecting an alteration in ESR1 in a sample from an individual having a skin ILC metastasis. In some embodiments, the alteration results in one or more amino acid substitutions in a polypeptide encoded by the ESR1 gene at amino acid position E380, V418, S463, V533, L536, Y537, D538, or any combination thereof, wherein the numbering of the residues is according to the amino acid sequence of SEQ ID NO: 29. In some embodiments, the one or more amino acid substitutions comprise a E380Q, V418E, S463P, V533M, L536Q, L536H, L536P, L536R, Y537S, Y537N, Y537C, Y537D, or D538G amino acid substitution, or any combination thereof, wherein the numbering of the residues is according to the amino acid sequence of SEQ ID NO: 29. In some embodiments, the alteration results in a deletion of amino acid V422 (V422del) and/or a deletion of amino acids V533-L536 (V533_L536del) in a polypeptide encoded by the ESR1 gene, wherein the numbering of the residues is according to the amino acid sequence of SEQ ID NO: 29.

In some embodiments, the method comprises detecting an alteration in ERBB2 in a sample from an individual having a liver ILC metastasis. In some embodiments, the method comprises detecting an alteration in ERBB2 in a sample from an individual having a bone ILC metastasis. In some embodiments, the method comprises detecting an alteration in ERBB2 in a sample from an individual having a skin ILC metastasis. In some embodiments, the alteration results in one or more amino acid substitutions in a polypeptide encoded by the ERBB2 gene at amino acid position S310, S653, V659, R678, V697, E717, T733, L755, I767, D769, G776, V777, T798, V842, L869, or any combination thereof, wherein the numbering of the residues is according to the amino acid sequence of SEQ ID NO: 34. In some embodiments, the one or more amino acid substitutions comprise a S310F, S310Y, S653C, V659D, R678Q, V697L, E717K, T733I, L755S, L755P, I767M, D769N, D769Y, D769H, G776V, V777L, T798I, V842I, or L869R amino acid substitution, or any combination thereof, wherein the numbering of the residues is according to the amino acid sequence of SEQ ID NO: 34. In some embodiments, the alteration results in a deletion of amino acids L755-T759 (L755_T759del) and/or amino acids L755-E757 (L755_E757del) in a polypeptide encoded by the ERBB2 gene, wherein the numbering of the residues is according to the amino acid sequence of SEQ ID NO: 34. In some embodiments, the alteration results in an insertion of one or more amino acid residues between amino acid residues A775 and G776, and/or between amino acid residues P780 and Y781 in a polypeptide encoded by the ERBB2 gene, wherein the numbering of the residues is according to the amino acid sequence of SEQ ID NO: 34. In some embodiments, the alteration results in an insertion of the amino acid sequence YVMA (SEQ ID NO: 51) between amino acid residues A775 and G776 (A775_G776insYVMA), and/or of the amino acid sequence GSP (SEQ ID NO: 52) between amino acid residues P780 and Y781 (P780_Y781insGSP), in a polypeptide encoded by the ERBB2 gene, wherein the numbering of the residues is according to the amino acid sequence of SEQ ID NO: 34.

In some embodiments, the method comprises detecting an alteration in ARID1A in a sample from an individual having a gastrointestinal ILC metastasis. In some embodiments, the method comprises detecting an alteration in ARID1A in a sample from an individual having a liver ILC metastasis. In some embodiments, the method comprises detecting an alteration in ARID1A in a sample from an individual having a female reproductive system ILC metastasis. In some embodiments, the method comprises detecting an alteration in ARID1A in a sample from an individual having a bone ILC metastasis. In some embodiments, the method comprises detecting an alteration in ARID1A in a sample from an individual having a skin ILC metastasis.

In some embodiments, the method comprises detecting an alteration in NF1 in a sample from an individual having a gastrointestinal ILC metastasis. In some embodiments, the method comprises detecting an alteration in NF1 in a sample from an individual having a liver ILC metastasis. In some embodiments, the method comprises detecting an alteration in NF1 in a sample from an individual having a female reproductive system ILC metastasis. In some embodiments, the method comprises detecting an alteration in NF1 in a sample from an individual having a bone ILC metastasis. In some embodiments, the method comprises detecting an alteration in NF1 in a sample from an individual having a skin ILC metastasis.

In some embodiments, the method comprises detecting an alteration in RB1 in a sample from an individual having a gastrointestinal ILC metastasis. In some embodiments, the method comprises detecting an alteration in RB1 in a sample from an individual having a liver ILC metastasis. In some embodiments, the method comprises detecting an alteration in RB1 in a sample from an individual having a skin ILC metastasis. In some embodiments, the method comprises detecting an alteration in RB1 in a sample from an individual having a bone ILC metastasis.

In some embodiments, the method comprises detecting an alteration in KRAS in a sample from an individual having a gastrointestinal ILC metastasis. In some embodiments, the method comprises detecting an alteration in KRAS in a sample from an individual having a liver ILC metastasis. In some embodiments, the method comprises detecting an alteration in KRAS in a sample from an individual having a bone ILC metastasis. In some embodiments, the alteration results in one or more amino acid substitutions in a polypeptide encoded by the KRAS gene at amino acid position G12, G13, L19, Q61, A146, K147, F156, or any combination thereof, wherein the numbering of the residues is according to the amino acid sequence of SEQ ID NO: 33. In some embodiments, the one or more amino acid substitutions comprise a G12V, G12D, G12A, G12R, G12S, G12C, G12L, G13D, L19F, Q61H, Q61K, Q61E, A146T, K147N, or F156L amino acid substitution, or any combination thereof, wherein the numbering of the residues is according to the amino acid sequence of SEQ ID NO: 33.

In some embodiments, the method comprises detecting an alteration in PTEN in a sample from an individual having a skin ILC metastasis. In some embodiments, the method comprises detecting an alteration in PTEN in a sample from an individual having a liver ILC metastasis. In some embodiments, the method comprises detecting an alteration in PTEN in a sample from an individual having a female reproductive system ILC metastasis.

In some embodiments, the method comprises detecting an alteration in NCOR1 in a sample from an individual having a female reproductive system ILC metastasis.

In some embodiments, the method comprises detecting an alteration in BRAF in a sample from an individual having a bone ILC metastasis. In some embodiments, the alteration results in one or more amino acid substitutions in a polypeptide encoded by the BRAF gene at amino acid position D380, G464, G466, S467, G469, L485, L584, E586, D594, V600, or any combination thereof, wherein the numbering of the residues is according to the amino acid sequence of SEQ ID NO: 35. In some embodiments, the one or more amino acid substitutions comprise a D380H, G464R, G466E, S467L, G469A, G469E, G469R, L485F, L584F, E586K, D594N, D594G, or V600E amino acid substitution, or any combination thereof, wherein the numbering of the residues is according to the amino acid sequence of SEQ ID NO: 35.

In some embodiments, the method comprises detecting an alteration in FGFR2 in a sample from an individual having a liver ILC metastasis. In some embodiments, the method comprises detecting an alteration in FGFR2 in a sample from an individual having a bone ILC metastasis. In some embodiments, the alteration results in one or more amino acid substitutions in a polypeptide encoded by the FGFR2 gene at amino acid position S252, P253, Y375, C382, M391, V395, M537, N549, K659, R664, or any combination thereof, wherein the numbering of the residues is according to the amino acid sequence of SEQ ID NO: 38. In some embodiments, the one or more amino acid substitutions comprise a S252W, P253R, Y375C, C382R, M391R, V395D, M537I, N549D, N549K, K659M, K659E, K659N, or R664W amino acid substitution, or any combination thereof, wherein the numbering of the residues is according to the amino acid sequence of SEQ ID NO: 38. In some embodiments, the alteration results in a frameshift of S799fs*22, wherein the numbering of the residues is according to the amino acid sequence of SEQ ID NO: 38.

In some embodiments, the method comprises detecting an alteration in TERT in a sample from an individual having a gastrointestinal ILC metastasis. In some embodiments, the alteration is an alteration in the promoter of TERT, wherein the alteration in the promoter of TERT comprises a −146C>T, −139_−138CC>TT, or −124C>T mutation, or any combination thereof, wherein the numbering of the nucleotides is according to SEQ ID NO: 22.

In some embodiments, the method comprises detecting an alteration in SMAD4 in a sample from an individual having a liver ILC metastasis.

In some embodiments, the alteration in the one or more genes is detected in the sample by one or more methods selected from a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping. In some embodiments, the sample from the individual comprises one or more proteins. In some embodiments, the alteration in the one or more genes is detected in a polypeptide encoded by the one or more genes. In some embodiments, the alteration is detected in the sample by one or more methods selected from immunoblotting, enzyme linked immunosorbent assay (ELISA), immunohistochemistry, or mass spectrometry.

In some embodiments, which may be combined with any of the preceding embodiments, the party is one or more of the individual, a caregiver, a physician, an oncologist, a hospital, a clinic, a third-party payor, an insurance company, or a government office. In some embodiments, the report is in electronic, web-based, and/or paper form. In some embodiments, the report identifies the presence or absence of the one or more biomarkers in a sample from the individual, and optionally comprises an identifier for the individual from which the sample was obtained. In some embodiments, the report comprises one or more of: (a) information on the role of the one or more biomarkers in disease; (b) information on prognosis, resistance, or potential or suggested therapeutic options; (c) information on the likely effectiveness of a therapeutic option, the acceptability of a therapeutic option, or the advisability of applying the therapeutic option to an individual; or (d) information, or a recommendation on, the administration of a drug. In some embodiments, the method further comprises obtaining the sample from the individual.

In another aspect, provided herein is a system, comprising: a memory configured to store one or more program instructions; and one or more processors configured to execute the one or more program instructions, the one or more program instructions when executed by the one or more processors are configured to: (a) obtain a plurality of sequence reads of one or more nucleic acids, wherein the one or more nucleic acids are derived from a sample obtained from an individual; (b) analyze the plurality of sequence reads for the presence of a tumor mutational burden (TMB) of at least about 10 mutations/megabase (mut/Mb); and (c) detect, based on the analyzing, a TMB of at least about 10 mut/Mb, in the sample. In some embodiments, the sample is a sample from an individual having an invasive lobular carcinoma (ILC) metastasis. In some embodiments, the analyzing is based on between about 0.8 Mb and about 1.1 Mb of sequenced nucleic acids. In some embodiments, the ILC metastasis is a bone ILC metastasis, a female reproductive system ILC metastasis, a gastrointestinal ILC metastasis, a liver ILC metastasis, or a skin ILC metastasis. In some embodiments, the plurality of sequence reads are obtained by whole exome sequencing, whole genome sequencing, or gene-targeted sequencing.

In another aspect, provided herein is a non-transitory computer readable storage medium comprising one or more programs executable by one or more computer processors for performing a method, comprising: (a) obtaining, using the one or more processors, a plurality of sequence reads of one or more nucleic acids, wherein the one or more nucleic acids are derived from a sample obtained from an individual; (b) analyzing, using the one or more processors, the plurality of sequence reads for the presence of a tumor mutational burden (TMB) of at least about 10 mutations/megabase (mut/Mb); and (c) detecting, using the one or more processors and based on the analyzing, a TMB of at least about 10 mut/Mb, in the sample. In some embodiments, the sample is a sample from an individual having an invasive lobular carcinoma (ILC) metastasis. In some embodiments, the analyzing is based on between about 0.8 Mb and about 1.1 Mb of sequenced nucleic acids. In some embodiments, the ILC metastasis is a bone ILC metastasis, a female reproductive system ILC metastasis, a gastrointestinal ILC metastasis, a liver ILC metastasis, or a skin ILC metastasis. In some embodiments, the plurality of sequence reads are obtained by whole exome sequencing, whole genome sequencing, or gene-targeted sequencing.

In another aspect, provided herein is a system, comprising: a memory configured to store one or more program instructions; and one or more processors configured to execute the one or more program instructions, the one or more program instructions when executed by the one or more processors are configured to: (a) obtain a plurality of sequence reads of one or more nucleic acids, wherein the one or more nucleic acids are derived from a sample obtained from an individual; (b) analyze the plurality of sequence reads for the presence of an alteration in one or more genes, wherein the one or more genes comprise PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, CDH1 or KMT2D; and (c) detect, based on the analyzing, an alteration in one or more genes, wherein the one or more genes comprise PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, CDH1 or KMT2D, in the sample. In some embodiments, the sample is a sample from an individual having an invasive lobular carcinoma (ILC) metastasis. In some embodiments, the ILC metastasis is a bone ILC metastasis, a female reproductive system ILC metastasis, a gastrointestinal ILC metastasis, a liver ILC metastasis, or a skin ILC metastasis. In some embodiments, the alteration comprises one or more of a substitution of one or more nucleotides, an insertion of one or more nucleotides, or a deletion of one or more nucleotides.

In some embodiments, the alteration is an alteration in PIK3CA. In some embodiments, the alteration results in one or more amino acid substitutions in a polypeptide encoded by the PIK3CA gene at amino acid position E81, R108, K111, G118, N345, D350, E365, E418, C420, E453, P539, E542, E545, Q546, E726, E970, M1004, M1043, N1044, H1047, G1049, or any combination thereof, wherein the numbering of the residues is according to the amino acid sequence of SEQ ID NO: 26. In some embodiments, the one or more amino acid substitutions comprise a E81K, R108H, K111N, G118D, N345K, D350N, E365K, E418K, C420R, E453K, E453Q, P539R, E542K, E545K, E545A, E545Q, Q546R, Q546K, E726K, E970K, M1004I, M1043I, N1044K, H1047R, H1047L, or G1049R amino acid substitution, or any combination thereof, wherein the numbering of the residues is according to the amino acid sequence of SEQ ID NO: 26. In some embodiments, the alteration results in a deletion of amino acid residue E110 (E110del) in a polypeptide encoded by the PIK3CA gene, wherein the numbering of the residues is according to the amino acid sequence of SEQ ID NO: 26.

In some embodiments, the alteration is an alteration in BRCA1 or BRCA2.

In some embodiments, the alteration is an alteration in ESR1. In some embodiments, the alteration results in one or more amino acid substitutions in a polypeptide encoded by the ESR1 gene at amino acid position E380, V418, S463, V533, L536, Y537, D538, or any combination thereof, wherein the numbering of the residues is according to the amino acid sequence of SEQ ID NO: 29. In some embodiments, the one or more amino acid substitutions comprise a E380Q, V418E, S463P, V533M, L536Q, L536H, L536P, L536R, Y537S, Y537N, Y537C, Y537D, or D538G amino acid substitution, or any combination thereof, wherein the numbering of the residues is according to the amino acid sequence of SEQ ID NO: 29. In some embodiments, the alteration results in a deletion of amino acid V422 (V422del) and/or a deletion of amino acids V533-L536 (V533_L536del) in a polypeptide encoded by the ESR1 gene, wherein the numbering of the residues is according to the amino acid sequence of SEQ ID NO: 29.

In some embodiments, the alteration is an alteration in NFL

In some embodiments, the alteration is an alteration in RB1.

In some embodiments, the alteration is an alteration in KRAS. In some embodiments, the alteration results in one or more amino acid substitutions in a polypeptide encoded by the KRAS gene at amino acid position G12, G13, L19, Q61, A146, K147, F156, or any combination thereof, wherein the numbering of the residues is according to the amino acid sequence of SEQ ID NO: 33. In some embodiments, the one or more amino acid substitutions comprise a G12V, G12D, G12A, G12R, G12S, G12C, G12L, G13D, L19F, Q61H, Q61K, Q61E, A146T, K147N, or F156L amino acid substitution, or any combination thereof, wherein the numbering of the residues is according to the amino acid sequence of SEQ ID NO: 33.

In some embodiments, the alteration is an alteration in ERBB2. In some embodiments, the alteration results in one or more amino acid substitutions in a polypeptide encoded by the ERBB2 gene at amino acid position S310, S653, V659, R678, V697, E717, T733, L755, I767, D769, G776, V777, T798, V842, L869, or any combination thereof, wherein the numbering of the residues is according to the amino acid sequence of SEQ ID NO: 34. In some embodiments, the one or more amino acid substitutions comprise a S310F, S310Y, S653C, V659D, R678Q, V697L, E717K, T733I, L755S, L755P, I767M, D769N, D769Y, D769H, G776V, V777L, T798I, V842I, or L869R amino acid substitution, or any combination thereof, wherein the numbering of the residues is according to the amino acid sequence of SEQ ID NO: 34. In some embodiments, the alteration results in a deletion of amino acids L755-T759 (L755_T759del) and/or amino acids L755-E757 (L755_E757del) in a polypeptide encoded by the ERBB2 gene, wherein the numbering of the residues is according to the amino acid sequence of SEQ ID NO: 34. In some embodiments, the alteration results in an insertion of one or more amino acid residues between amino acid residues A775 and G776, and/or between amino acid residues P780 and Y781 in a polypeptide encoded by the ERBB2 gene, wherein the numbering of the residues is according to the amino acid sequence of SEQ ID NO: 34. In some embodiments, the alteration results in an insertion of the amino acid sequence YVMA (SEQ ID NO: 51) between amino acid residues A775 and G776 (A775_G776insYVMA), and/or of the amino acid sequence GSP (SEQ ID NO: 52) between amino acid residues P780 and Y781 (P780_Y781insGSP), in a polypeptide encoded by the ERBB2 gene, wherein the numbering of the residues is according to the amino acid sequence of SEQ ID NO: 34.

In some embodiments, the alteration is an alteration in BRAF. In some embodiments, the alteration results in one or more amino acid substitutions in a polypeptide encoded by the BRAF gene at amino acid position D380, G464, G466, S467, G469, L485, L584, E586, D594, V600, or any combination thereof, wherein the numbering of the residues is according to the amino acid sequence of SEQ ID NO: 35. In some embodiments, the one or more amino acid substitutions comprise a D380H, G464R, G466E, S467L, G469A, G469E, G469R, L485F, L584F, E586K, D594N, D594G, or V600E amino acid substitution, or any combination thereof, wherein the numbering of the residues is according to the amino acid sequence of SEQ ID NO: 35.

In some embodiments, the alteration is an alteration in ARID1A.

In some embodiments, the alteration is an alteration in PTEN. In some embodiments, the alteration is a PTEN deletion.

In some embodiments, the alteration is an alteration in FGFR2. In some embodiments, the alteration results in one or more amino acid substitutions in a polypeptide encoded by the FGFR2 gene at amino acid position S252, P253, Y375, C382, M391, V395, M537, N549, K659, R664, or any combination thereof, wherein the numbering of the residues is according to the amino acid sequence of SEQ ID NO: 38. In some embodiments, the one or more amino acid substitutions comprise a S252W, P253R, Y375C, C382R, M391R, V395D, M537I, N549D, N549K, K659M, K659E, K659N, or R664W amino acid substitution, or any combination thereof, wherein the numbering of the residues is according to the amino acid sequence of SEQ ID NO: 38. In some embodiments, the alteration results in a frameshift of S799fs*22, wherein the numbering of the residues is according to the amino acid sequence of SEQ ID NO: 38.

In some embodiments, the alteration is an alteration in SMAD4.

In some embodiments, the alteration is an alteration in PTPN11.

In some embodiments, the alteration is an alteration in TERT. In some embodiments, the alteration is an alteration in the promoter of TERT, wherein the alteration in the promoter of TERT comprises a −146C>T, −139_−138CC>TT, or −124C>T mutation, or any combination thereof, wherein the numbering of the nucleotides is according to SEQ ID NO: 22.

In some embodiments, the alteration is an alteration in ALK.

In some embodiments, the alteration is an alteration in NCOR1.

In some embodiments, the alteration is an alteration in APC.

In some embodiments, the alteration is an alteration in CDH1. In some embodiments, the alteration comprises one or more deleterious CDH1 mutations. In some embodiments, the one or more deleterious CDH1 mutations result in loss of function of a CDH1 polypeptide. In some embodiments, the one or more deleterious CDH1 mutations result in a mutation of the CDH1 start codon. In some embodiments, the one or more deleterious CDH1 mutations result in one or more amino acid substitutions in a CDH1 polypeptide at amino acid position E243, D402, D433, A634, or any combination thereof, wherein the numbering of the residues is according to the amino acid sequence of SEQ ID NO: 50. In some embodiments, the one or more amino acid substitutions comprise a E243K, D402N, D433N, or A634V amino acid substitution, or any combination thereof, wherein the numbering of the residues is according to the amino acid sequence of SEQ ID NO: 50. In some embodiments, the one or more deleterious CDH1 mutations result in a stop codon at amino acid position W4 (W4*), S9 (S9*), Q16 (Q16*), W20 (W20*), Q23 (Q23*), E24 (E24*), E26 (E26*), E35 (E35*), Y37 (Y37*), E47 (E47*), R54 (R54*), E58 (E58*), R63 (R63*), Q64 (Q64*), R74 (R74*), W103 (W103*), Q129 (Q129*), R150 (R150*), Q152 (Q152*), Q177 (Q177*), Y190 (Y190*), Y228 (Y228*), E243 (E243*), Q255 (Q255*), Q264 (Q264*), Y302 (Y302*), R335 (R335*), Q346 (Q346*), E353 (E353*), Q383 (Q383*), Q449 (Q449*), E463 (E463*), Y523 (Y523*), W526 (W526*), R598 (R598*), Q610 (Q610*), Q641 (Q641*), E648 (E648*), Q699 (Q699*), Q706 (Q706*), Q765 (Q765*), Q771 (Q771*), E806 (E806*), Y827 (Y827*), or any combination thereof, wherein the numbering of the residues is according to the amino acid sequence of SEQ ID NO: 50. In some embodiments, the one or more deleterious CDH1 mutations comprise a frameshift, wherein the frameshift is an T115fs*53, P127fs*41, P200fs*6, P200fs*16, V202fs*7, H233fs*11, P277fs*5, Y302fs*1, S337fs*12, Y523fs*1, L585fs*4, 1650fs*3, 1650fs*13, A719fs*29, or Q765fs*4 frameshift, wherein the numbering of the residues is according to the amino acid sequence of SEQ ID NO: 50. In some embodiments, the one or more deleterious CDH1 mutations comprise a splice site mutation, wherein the splice site mutation is a 48+1G>A, 1565+1G>A, or 1565+1G>T splice site mutation, wherein the numbering of the nucleotides is according to SEQ ID NO: 49.

In some embodiments, the plurality of sequence reads is obtained by sequencing, whole exome sequencing, whole genome sequencing, gene-targeted sequencing, or next-generation sequencing.

In another aspect, provided herein is a non-transitory computer readable storage medium comprising one or more programs executable by one or more computer processors for performing a method, comprising: (a) obtaining, using the one or more processors, a plurality of sequence reads of one or more nucleic acids, wherein the one or more nucleic acids are derived from a sample obtained from an individual; (b) analyzing, using the one or more processors, the plurality of sequence reads for the presence of an alteration in one or more genes, wherein the one or more genes comprise PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, CDH1 or KMT2D; and (c) detecting, using the one or more processors and based on the analyzing, an alteration in one or more genes, wherein the one or more genes comprise PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, CDH1 or KMT2D, in the sample. In some embodiments, the sample is a sample from an individual having an invasive lobular carcinoma (ILC) metastasis. In some embodiments, the ILC metastasis is a bone ILC metastasis, a female reproductive system ILC metastasis, a gastrointestinal ILC metastasis, a liver ILC metastasis, or a skin ILC metastasis. In some embodiments, the alteration comprises one or more of a substitution of one or more nucleotides, an insertion of one or more nucleotides, or a deletion of one or more nucleotides.

In some embodiments, the alteration is an alteration in PIK3CA. In some embodiments, the alteration results in one or more amino acid substitutions in a polypeptide encoded by the PIK3CA gene at amino acid position E81, R108, K111, G118, N345, D350, E365, E418, C420, E453, P539, E542, E545, Q546, E726, E970, M1004, M1043, N1044, H1047, G1049, or any combination thereof, wherein the numbering of the residues is according to the amino acid sequence of SEQ ID NO: 26. In some embodiments, the one or more amino acid substitutions comprise a E81K, R108H, K111N, G118D, N345K, D350N, E365K, E418K, C420R, E453K, E453Q, P539R, E542K, E545K, E545A, E545Q, Q546R, Q546K, E726K, E970K, M1004I, M1043I, N1044K, H1047R, H1047L, or G1049R amino acid substitution, or any combination thereof, wherein the numbering of the residues is according to the amino acid sequence of SEQ ID NO: 26. In some embodiments, the alteration results in a deletion of amino acid residue E110 (E110del) in a polypeptide encoded by the PIK3CA gene, wherein the numbering of the residues is according to the amino acid sequence of SEQ ID NO: 26.

In some embodiments, the alteration is an alteration in BRCA1 or BRCA2.

In some embodiments, the alteration is an alteration in ESR1. In some embodiments, the alteration results in one or more amino acid substitutions in a polypeptide encoded by the ESR1 gene at amino acid position E380, V418, S463, V533, L536, Y537, D538, or any combination thereof, wherein the numbering of the residues is according to the amino acid sequence of SEQ ID NO: 29. In some embodiments, the one or more amino acid substitutions comprise a E380Q, V418E, S463P, V533M, L536Q, L536H, L536P, L536R, Y537S, Y537N, Y537C, Y537D, or D538G amino acid substitution, or any combination thereof, wherein the numbering of the residues is according to the amino acid sequence of SEQ ID NO: 29. In some embodiments, the alteration results in a deletion of amino acid V422 (V422del) and/or a deletion of amino acids V533-L536 (V533_L536del) in a polypeptide encoded by the ESR1 gene, wherein the numbering of the residues is according to the amino acid sequence of SEQ ID NO: 29.

In some embodiments, the alteration is an alteration in NFL

In some embodiments, the alteration is an alteration in RB1.

In some embodiments, the alteration is an alteration in KRAS. In some embodiments, the alteration results in one or more amino acid substitutions in a polypeptide encoded by the KRAS gene at amino acid position G12, G13, L19, Q61, A146, K147, F156, or any combination thereof, wherein the numbering of the residues is according to the amino acid sequence of SEQ ID NO: 33. In some embodiments, the one or more amino acid substitutions comprise a G12V, G12D, G12A, G12R, G12S, G12C, G12L, G13D, L19F, Q61H, Q61K, Q61E, A146T, K147N, or F156L amino acid substitution, or any combination thereof, wherein the numbering of the residues is according to the amino acid sequence of SEQ ID NO: 33.

In some embodiments, the alteration is an alteration in ERBB2. In some embodiments, the alteration results in one or more amino acid substitutions in a polypeptide encoded by the ERBB2 gene at amino acid position S310, S653, V659, R678, V697, E717, T733, L755, I767, D769, G776, V777, T798, V842, L869, or any combination thereof, wherein the numbering of the residues is according to the amino acid sequence of SEQ ID NO: 34. In some embodiments, the one or more amino acid substitutions comprise a S310F, S310Y, S653C, V659D, R678Q, V697L, E717K, T733I, L755S, L755P, I767M, D769N, D769Y, D769H, G776V, V777L, T798I, V842I, or L869R amino acid substitution, or any combination thereof, wherein the numbering of the residues is according to the amino acid sequence of SEQ ID NO: 34. In some embodiments, the alteration results in a deletion of amino acids L755-T759 (L755_T759del) and/or amino acids L755-E757 (L755_E757del) in a polypeptide encoded by the ERBB2 gene, wherein the numbering of the residues is according to the amino acid sequence of SEQ ID NO: 34. In some embodiments, the alteration results in an insertion of one or more amino acid residues between amino acid residues A775 and G776, and/or between amino acid residues P780 and Y781 in a polypeptide encoded by the ERBB2 gene, wherein the numbering of the residues is according to the amino acid sequence of SEQ ID NO: 34. In some embodiments, the alteration results in an insertion of the amino acid sequence YVMA (SEQ ID NO: 51) between amino acid residues A775 and G776 (A775_G776insYVMA), and/or of the amino acid sequence GSP (SEQ ID NO: 52) between amino acid residues P780 and Y781 (P780_Y781insGSP), in a polypeptide encoded by the ERBB2 gene, wherein the numbering of the residues is according to the amino acid sequence of SEQ ID NO: 34.

In some embodiments, the alteration is an alteration in BRAF. In some embodiments, the alteration results in one or more amino acid substitutions in a polypeptide encoded by the BRAF gene at amino acid position D380, G464, G466, S467, G469, L485, L584, E586, D594, V600, or any combination thereof, wherein the numbering of the residues is according to the amino acid sequence of SEQ ID NO: 35. In some embodiments, the one or more amino acid substitutions comprise a D380H, G464R, G466E, S467L, G469A, G469E, G469R, L485F, L584F, E586K, D594N, D594G, or V600E amino acid substitution, or any combination thereof, wherein the numbering of the residues is according to the amino acid sequence of SEQ ID NO: 35.

In some embodiments, the alteration is an alteration in ARID1A.

In some embodiments, the alteration is an alteration in PTEN. In some embodiments, the alteration is a PTEN deletion.

In some embodiments, the alteration is an alteration in FGFR2. In some embodiments, the alteration results in one or more amino acid substitutions in a polypeptide encoded by the FGFR2 gene at amino acid position S252, P253, Y375, C382, M391, V395, M537, N549, K659, R664, or any combination thereof, wherein the numbering of the residues is according to the amino acid sequence of SEQ ID NO: 38. In some embodiments, the one or more amino acid substitutions comprise a S252W, P253R, Y375C, C382R, M391R, V395D, M537I, N549D, N549K, K659M, K659E, K659N, or R664W amino acid substitution, or any combination thereof, wherein the numbering of the residues is according to the amino acid sequence of SEQ ID NO: 38. In some embodiments, the alteration results in a frameshift of S799fs*22, wherein the numbering of the residues is according to the amino acid sequence of SEQ ID NO: 38.

In some embodiments, the alteration is an alteration in SMAD4.

In some embodiments, the alteration is an alteration in PTPN11.

In some embodiments, the alteration is an alteration in TERT. In some embodiments, the alteration is an alteration in the promoter of TERT, wherein the alteration in the promoter of TERT comprises a −146C>T, −139_−138CC>TT, or −124C>T mutation, or any combination thereof, wherein the numbering of the nucleotides is according to SEQ ID NO: 22.

In some embodiments, the alteration is an alteration in ALK.

In some embodiments, the alteration is an alteration in NCOR1.

In some embodiments, the alteration is an alteration in APC.

In some embodiments, the alteration is an alteration in CDH1. In some embodiments, the alteration comprises one or more deleterious CDH1 mutations. In some embodiments, the one or more deleterious CDH1 mutations result in loss of function of a CDH1 polypeptide. In some embodiments, the one or more deleterious CDH1 mutations result in a mutation of the CDH1 start codon. In some embodiments, the one or more deleterious CDH1 mutations result in one or more amino acid substitutions in a CDH1 polypeptide at amino acid position E243, D402, D433, A634, or any combination thereof, wherein the numbering of the residues is according to the amino acid sequence of SEQ ID NO: 50. In some embodiments, the one or more amino acid substitutions comprise a E243K, D402N, D433N, or A634V amino acid substitution, or any combination thereof, wherein the numbering of the residues is according to the amino acid sequence of SEQ ID NO: 50. In some embodiments, the one or more deleterious CDH1 mutations result in a stop codon at amino acid position W4 (W4*), S9 (S9*), Q16 (Q16*), W20 (W20*), Q23 (Q23*), E24 (E24*), E26 (E26*), E35 (E35*), Y37 (Y37*), E47 (E47*), R54 (R54*), E58 (E58*), R63 (R63*), Q64 (Q64*), R74 (R74*), W103 (W103*), Q129 (Q129*), R150 (R150*), Q152 (Q152*), Q177 (Q177*), Y190 (Y190*), Y228 (Y228*), E243 (E243*), Q255 (Q255*), Q264 (Q264*), Y302 (Y302*), R335 (R335*), Q346 (Q346*), E353 (E353*), Q383 (Q383*), Q449 (Q449*), E463 (E463*), Y523 (Y523*), W526 (W526*), R598 (R598*), Q610 (Q610*), Q641 (Q641*), E648 (E648*), Q699 (Q699*), Q706 (Q706*), Q765 (Q765*), Q771 (Q771*), E806 (E806*), Y827 (Y827*), or any combination thereof, wherein the numbering of the residues is according to the amino acid sequence of SEQ ID NO: 50. In some embodiments, the one or more deleterious CDH1 mutations comprise a frameshift, wherein the frameshift is an T115fs*53, P127fs*41, P200fs*6, P200fs*16, V202fs*7, H233fs*11, P277fs*5, Y302fs*1, S337fs*12, Y523fs*1, L585fs*4, I650fs*3, I650fs*13, A719fs*29, or Q765fs*4 frameshift, wherein the numbering of the residues is according to the amino acid sequence of SEQ ID NO: 50. In some embodiments, the one or more deleterious CDH1 mutations comprise a splice site mutation, wherein the splice site mutation is a 48+1G>A, 1565+1G>A, or 1565+1G>T splice site mutation, wherein the numbering of the nucleotides is according to SEQ ID NO: 49.

In some embodiments, the plurality of sequence reads is obtained by sequencing, whole exome sequencing, whole genome sequencing, gene-targeted sequencing, or next-generation sequencing.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIGS. 1A-1B depict the comprehensive genomic profiling (CGP) workflow for analysis of invasive lobular carcinoma (ILC) patient samples. FIG. 1A is a schematic of the CGP workflow. “FFPE” refers to formalin-fixed, paraffin-embedded samples. FIG. 1B is a schematic of the ILC patient sample analysis workflow. A set of ILC patients (defined by histology or CDH1 deleterious gene mutation) was identified. Samples from the patients were bucketed based on metastatic biopsy site (gastrointestinal, female reproductive, liver, skin, or bone). The prevalence of immune checkpoint inhibitor (ICPI) biomarkers (tumor mutational burden (TMB), PD-L1 infiltrating cell (IC) staining) and genomic alterations was then examined to determine if there were differences between the ILC populations at each metastatic site.

FIG. 2 is a summary of the results from the ILC patient sample CGP analysis. The summary lists the prevalence of metastasis-enriched alterations in breast-biopsied ILC/invasive ductal carcinoma (IDC), metastatic IDC/ILC, and ILC metastases (mets) broken down by metastatic site. All gene alteration classes that were significantly enriched in metastatic ILC overall, or in at least one met site, relative to breast-biopsied ILC, are shown. Alterations in ICPI biomarkers and therapy-associated sites are also shown. “Mut” indicates short variant alterations (e.g., a substitution of one or more nucleotides, an insertion of one or more nucleotides, or a deletion of one or more nucleotides) and “RE” indicates rearrangement/fusion events.

FIG. 3 shows the differences in biomarkers of immune checkpoint inhibitor response in ILC and IDC metastases (mets), and across ILC met sites. The top panel shows the fraction of patients with tumor mutational burden (TMB) greater or equal to 10 mutations/megabase (>10 mut/Mb) for all invasive ductal carcinoma (IDC) mets, for all ILC mets, and for ILC mets stratified by site. The bottom panel shows PD-L1 IC positivity rates (VENTANA SP142-IC) for all IDC mets, for all ILC mets, and for ILC mets stratified by site. The highest ICPI biomarker prevalence was observed in gastrointestinal and skin ILC mets. “fem repr” indicates female reproductive mets and “gastro int” indicates gastrointestinal mets.

FIG. 4 shows the prevalence of potentially actionable alterations in ILC and IDC metastases (mets), and across ILC met sites. PIK3CA short variant alteration prevalence was similarly high across ILC met sites. The BRCA1/2 alteration prevalence differed across ILC mets, with the highest prevalence in female reproductive ILC mets and the lowest prevalence in gastrointestinal ILC mets. “fem repr” indicates female reproductive mets and “gastro in” indicates gastrointestinal mets.

FIG. 5 shows a longtail analysis of alteration prevalence in local ILC (breast-biopsied) and metastatic ILC. All gene alterations that were significantly enriched in metastatic ILC overall or in at least one met site, relative to breast-biopsied ILC, are shown. “met” indicates metastatic ILC, while “breast” indicates breast-biopsied ILC.

FIGS. 6A-6B show the frequency of metastasis-enriched alterations in ILC and IDC breast biopsies, ILC and IDC metastases (mets), and across ILC met sites. FIG. 6A shows the fraction of samples with at least one metastasis-enriched (ME) alteration. Female reproductive tumors exhibited the lowest prevalence of ME alterations, while liver mets had the highest prevalence. FIG. 6B shows the prevalence of select ME alterations in ILC and IDC mets, and across ILC met sites. Notable heterogeneity was observed in ERBB2, ESR1, NF1, and RB1 across met sites. “fem repr” indicates female reproductive mets and “gastro in” indicates gastrointestinal mets.

FIG. 7 depicts an exemplary device, in accordance with some embodiments.

FIG. 8 depicts an exemplary system, in accordance with some embodiments.

FIG. 9 depicts a block diagram of an exemplary process for detecting a tumor mutational burden (TMB) of at least about 10 mutations/megabase (mut/Mb), in accordance with some embodiments.

FIG. 10 depicts a block diagram of an exemplary process for detecting an alteration in one or more genes, e.g., in one or more of PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, CDH1 or KMT2D, in accordance with some embodiments.

### DETAILED DESCRIPTION OF THE INVENTION

Provided herein are methods of treating or delaying progression of metastatic invasive lobular carcinoma (ILC). In some embodiments, the methods comprise acquiring knowledge of the presence of one or more biomarkers in a sample from an individual having an ILC metastasis. In some embodiments, the one or more biomarkers are selected from a high tumor mutational burden, e.g., of at least about 10 mutations/megabase (mut/Mb), a PD-L1-positive ILC metastasis, or an alteration in one or more genes. In some embodiments, the one or more genes are selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D. In some embodiments, the methods comprise acquiring knowledge that an ILC metastasis has a high tumor mutational burden, e.g., of at least about 10 mutations/megabase (mut/Mb), in a sample from an individual having an ILC metastasis. In some embodiments, the methods further comprise administering to the individual an effective amount of an immune checkpoint inhibitor responsive to acquiring knowledge of a high tumor mutational burden, e.g., of at least about 10 mutations/megabase (mut/Mb), in a sample from the individual. In some embodiments, the methods comprise acquiring knowledge of a PD-L1-positive ILC metastasis in a sample from an individual having an ILC metastasis. In some embodiments, the methods further comprise administering to the individual an effective amount of an immune checkpoint inhibitor responsive to acquiring knowledge of a PD-L1-positive ILC metastasis in a sample from the individual. In some embodiments, the methods comprise acquiring knowledge of an alteration in one or more genes in a sample from an individual having an ILC metastasis, wherein the one or more genes are selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D. In some embodiments, the methods further comprise administering to the individual an effective amount of an anti-cancer agent responsive to acquiring knowledge of an alteration in one or more genes in a sample from the individual, wherein the one or more genes are selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D. In some embodiments, acquiring knowledge of the presence of the one or more biomarkers in a sample from an individual having an ILC metastasis comprises detecting the one or more biomarkers in the sample. In some embodiments, the one or more biomarkers are detected in cancer cells (e.g., in tumor cells, such as tumor cells from an ILC metastasis described herein), or in tumor infiltrating immune cells.

Also provided herein are methods for genomic profiling of an invasive lobular carcinoma (ILC) metastasis. In some embodiments, the methods comprise detecting one or more biomarkers in a sample from an individual having an ILC metastasis. In some embodiments, the methods comprise detecting a high tumor mutational burden (TMB), e.g., of at least about 10 mutations/megabase (mut/Mb), in a sample from an individual having an ILC metastasis. In some embodiments, the methods comprise detecting a PD-L1-positive ILC metastasis in a sample from an individual having an ILC metastasis. In some embodiments, the methods comprise detecting an alteration in one or more genes in a sample from an individual having an ILC metastasis, wherein the one or more genes are selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D. In some embodiments, the methods further comprise providing a report to a party.

### Definitions

Before describing the invention in detail, it is to be understood that this invention is not limited to particular compositions or biological systems. It is also to be understood that the terminology used herein is for the purpose of describing particular embodiments only, and is not intended to be limiting.

As used in this specification and the appended claims, the singular forms “a”, “an” and “the” include plural referents unless the context clearly dictates otherwise. Thus, for example, reference to “a molecule” optionally includes a combination of two or more such molecules, and the like.

The term “or” is used herein to mean, and is used interchangeably with, the term “and/or”, unless context clearly indicates otherwise.

The terms “about” or “approximately” as used herein refer to the usual error range for the respective value readily known to the skilled person in this technical field, for example, an acceptable degree of error or deviation for the quantity measured given the nature or precision of the measurements. Reference to “about” or “approximately” a value or parameter herein includes (and describes) embodiments that are directed to that value or parameter per se.

The term “fusion” or “gene fusion” is used generically herein, and includes any fusion molecule (e.g., a gene (e.g., in genomic DNA), a gene product (e.g., cDNA, mRNA, polypeptide, protein), and variants thereof) that includes a fragment of a first gene or gene product and a fragment of a second gene or gene product described herein. A fusion molecule includes a “breakpoint” or “fusion junction,” which is the transition (i.e., direct fusion) point between the first gene or gene product, or fragment thereof, and the second gene or gene product, or fragment thereof.

The term “rearrangement” includes any a deletion, inversion, insertion, duplication, fusion, translocation, or other chromosomal or genomic rearrangement. In some cases, rearrangements can be caused by breakage of a nucleic acid, e.g., genomic DNA, followed by rejoining of broken ends to produce a new arrangement, e.g., a new chromosomal arrangement of genes. Deletions can include deletions of entire chromosomes or deletions of fragments of one or more chromosomes; duplications can include duplications of entire chromosomes, or of regions smaller than an entire chromosome; translocations can include non-reciprocal translocations or balanced translocations; and inversions can include intra-chromosomal inversions, paracentric inversions, or pericentric inversions.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual is a human. In some embodiments, the individual is a human patient, e.g., a human patient having a cancer described herein, and/or a biomarker or an alteration described herein.

An “effective amount” or a “therapeutically effective amount” of an agent, e.g., an anti-cancer agent, or a pharmaceutical formulation, refers to an amount effective, at dosages and for periods of time necessary, to achieve the desired therapeutic or prophylactic result, e.g., in the treatment or management of a cancer, for example, delaying or minimizing one or more symptoms associated with the cancer. In some embodiments, an effective amount or a therapeutically effective amount of an agent refers to an amount of the agent at dosages and for periods of time necessary, alone or in combination with other therapeutic agents, which provides a therapeutic or prophylactic benefit in the treatment or management of a disease such as a cancer. In some embodiments, an effective amount or a therapeutically effective amount of an agent enhances the therapeutic or prophylactic efficacy of another therapeutic agent or another therapeutic modality.

As used herein, “tumor mutation burden” or “tumor mutational burden” refer to the total number of nonsynonymous mutations per coding area of a tumor genome. For example, tumor mutational burden may be expressed as the number of mutations per megabase (mut/Mb).

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, delaying progression of disease, alleviation of symptoms, diminishment of any direct or indirect pathological consequences of the disease, decreasing the rate of disease progression, amelioration or palliation of the disease state, and remission or improved prognosis. In some embodiments, the terms “treatment,” “treat,” or “treating” include preventing a disease, such as cancer, e.g., before an individual begins to suffer from a cancer or from re-growth or recurrence of the cancer. In some embodiments, the terms “treatment,” “treat,” or “treating” include inhibiting or reducing the severity of a disease such as a cancer.

“Sample,” as used herein, refers to a biological sample obtained or derived from a source of interest, as described herein.

### Methods of the Disclosure

In certain aspects, provided herein are methods of treating or delaying progression of invasive lobular carcinoma (ILC) metastasis in an individual.

In some embodiments, the methods comprise acquiring knowledge that an ILC metastasis has a high tumor mutational burden in a sample from an individual having an ILC metastasis. In some embodiments, the ILC metastasis has a tumor mutational burden (TMB) of at least about 10 mutations/megabase (mut/Mb). In some embodiments, the methods comprise administering an effective amount of an immune checkpoint inhibitor responsive to knowledge that the ILC metastasis has a high tumor mutational burden, e.g., of at least about 10 mut/Mb.

In some embodiments, the methods comprise acquiring knowledge that an ILC metastasis is PD-L1-positive in a sample from an individual having an ILC metastasis. In some embodiments, the methods comprise administering an effective amount of an immune checkpoint inhibitor responsive to knowledge that the ILC metastasis is PD-L1-positive.

In some embodiments, the methods comprise acquiring knowledge of an alteration in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D in a sample from an individual having an ILC metastasis. In some embodiments, the methods comprise administering an effective amount of an anti-cancer agent responsive to knowledge of the presence of an alteration in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D in a sample from an individual having an ILC metastasis.

Also provided herein are methods for genomic profiling of an invasive lobular carcinoma (ILC) metastasis. In some embodiments, the methods comprise detecting one or more biomarkers in a sample from an individual having an ILC metastasis. In some embodiments, the methods further comprise providing a report to a party. In some embodiments, the one or more biomarkers include tumor mutational burden, PD-L1-positive status, and/or an alteration in one or more genes. In some embodiments, the one or more genes are selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D. In some embodiments, the methods for genomic profiling of an ILC metastasis comprise detecting a high tumor mutational burden in a sample from an individual having an ILC metastasis. In some embodiments, a high tumor mutational burden comprises a tumor mutational burden (TMB) of at least about 10 mutations/megabase (mut/Mb). In some embodiments, the methods for genomic profiling of an ILC metastasis comprise detecting a tumor mutational burden (TMB) of at least about 10 mutations/megabase (mut/Mb). In some embodiments, the methods for genomic profiling of an ILC metastasis comprise detecting a PD-L1-positive ILC metastasis.

In some embodiments, the individual is a human. In some embodiments, the ILC metastasis is a bone ILC metastasis, a female reproductive system ILC metastasis, a gastrointestinal ILC metastasis, a liver ILC metastasis, or a skin ILC metastasis.

Invasive Lobular Carcinoma

Invasive lobular carcinoma (ILC) is a type of breast cancer that originates in the milk-producing glands or lobules in the breast, but that has escaped out of the lobule. Once out of the lobule, invasive lobular carcinoma cells have the potential to spread to the lymph nodes and metastasize to other parts of the body.

ILCs generally have a morphology characterized by uniform cells with spherical nuclei and indistinct nucleoli. Most ILCs are classified as luminal A breast cancer and are estrogen receptor (ER)-positive, progesterone receptor (PR)-positive, have a low proliferation index, and are HER2 negative. Triple negative ILC (ER, PR, and HER2 negative) occurs less frequently and has a unique morphology, referred to as the pleomorphic histological subtype. This subtype includes larger cells with expansive eosinophilic cytoplasm. Some cases of ILC are HER2 positive. Most ILCs have loss of expression of E-cadherin protein, which is a transmembrane protein that mediates intercellular adhesion and polarity. Thus, loss of E-cadherin expression is believed to result in loss of intercellular adhesion and the characteristic discohesive and single-file pattern of ILC cells. In some cases, loss of E-cadherin expression occurs through one or more mutations in the CDH1 gene. In some cases, loss of E-cadherin expression occurs through methylation of the CDH1 gene. In some cases, loss of E-cadherin expression occurs through loss of heterozygosity in the chromosome region 16q. In some cases, loss of E-cadherin expression occurs through one or more mutations in the CDH1 gene and loss of heterozygosity in the chromosome region 16q. In some cases, loss of E-cadherin occurs through dysregulated expression of catenin-binding proteins (a, b, c and p120-catenin). See, e.g., Luveta et al., Oncol Ther (2020) 8:1-11 for a review of ILC. An exemplary nucleic acid sequence encoding CDH1 is available as Transcript ID NM_004360.5, provided herein as SEQ ID NO: 49, and available at the website: www[dot]ncbi[dot]nlm[dot]nih[dot]gov/nuccore/1519311738.

An exemplary amino acid sequence of a CDH1 polypeptide is provided herein as SEQ ID NO: 50.

(SEQ ID NO: 50)
MGPWSRSLSALLLLLQVSSWLCQEPEPCHPGFDAESYTFTVPRRHLERGRVLGRVNFEDCTGRQRTAY
FSLDTRFKVGTDGVITVKRPLRFHNPQIHFLVYAWDSTYRKESTKVTLNTVGHHHRPPPHQASVSGIQ
AELLTFPNSSPGLRRQKRDWVIPPISCPENEKGPFPKNLVQIKSNKDKEGKVFYSITGQGADTPPVGV
FIIERETGWLKVTEPLDRERIATYTLFSHAVSSNGNAVEDPMEILITVTDQNDNKPEFTQEVFKGSVM
EGALPGTSVMEVTATDADDDVNTYNAAIAYTILSQDPELPDKNMFTINRNTGVISVVTTGLDRESEPT
YTLVVQAADLQGEGLSTTATAVITVTDTNDNPPIFNPTTYKGQVPENEANVVITTLKVTDADAPNTPA
WEAVYTILNDDGGQFVVTTNPVNNDGILKTAKGLDFEAKQQYILHVAVTNVVPFEVSLTTSTATVTVD
VLDVNEAPIFVPPEKRVEVSEDFGVGQEITSYTAQEPDTFMEQKITYRIWRDTANWLEINPDTGAIST
RAELDREDFEHVKNSTYTALIIATDNGSPVATGTGTLLLILSDVNDNAPIPEPRTIFFCERNPKPQVI
NIIDADLPPNTSPFTAELTHGASANWTIQYNDPTQESIILKPKMALEVGDYKINLKLMDNQNKDQVTT
LEVSVCDCEGAAGVCRKAQPVEAGLQIPAILGILGGILALLILILLLLLFLRRRAVVKEPLLPPEDDT
RDNVYYYDEEGGGEEDQDFDLSQLHRGLDARPEVTRNDVAPTLMSVPRYLPRPANPDEIGNFIDENLK
AADTDPTAPPYDSLLVEDYEGSGSEAASLSSLNSSESDKDQDYDYLNEWGNRFKKLADMYGGGEDD

ILC has a high propensity for widespread metastasis, and is thus associated with worse long-term outcomes. For example, ILC can metastasize to bones, the female reproductive system (e.g., cervix, ovaries, uterus, vagina, vulva, endometrium, or fallopian tubes), the gastrointestinal tract (e.g., stomach, esophagus, anus, duodenum, colon, small intestine, jejunum, rectum, colon and rectum, pancreas, biliary system, or gall bladder), liver, brain, lungs, omentum, pleural fluid, spine, peritoneal fluid, bone marrow, bladder, pleura, iliac crest, head and neck, abdomen, pelvis, abdominal wall, retroperitoneum, mediastinum, adrenal gland, appendix, muscle, eye, pericardial fluid, pericardium, thyroid gland, kidney, gastro-esophageal junction, ureter, diaphragm, or skin.

In some embodiments, an ILC metastasis comprises one or more deleterious CDH1 mutations. In some embodiments, the one or more deleterious CDH1 mutations result in loss of function of the CDH1 polypeptide. In some embodiments, the one or more deleterious CDH1 mutations comprise one or more nonsense mutations in a nucleic acid sequence encoding a CDH1 polypeptide. In some embodiments, the one or more deleterious CDH1 mutations result in a mutation of the CDH1 start codon. In some embodiments, the one or more deleterious CDH1 mutations result in one or more amino acid substitutions in a CDH1 polypeptide at amino acid position E243, D402, D433, A634, or any combination thereof, wherein the numbering of the residues is according to SEQ ID NO: 50, or according to the amino acid sequence of a human CDH1 polypeptide. In some embodiments, the one or more amino acid substitutions comprise a E243K, D402N, D433N, or A634V amino acid substitution, or any combination thereof, wherein the numbering of the residues is according to SEQ ID NO: 50, or according to the amino acid sequence of a human CDH1 polypeptide. In some embodiments, the one or more deleterious CDH1 mutations result in a stop codon at amino acid position W4 (W4*), S9 (S9*), Q16 (Q16*), W20 (W20*), Q23 (Q23*), E24 (E24*), E26 (E26*), E35 (E35*), Y37 (Y37*), E47 (E47*), R54 (R54*), E58 (E58*), R63 (R63*), Q64 (Q64*), R74 (R74*), W103 (W103*), Q129 (Q129*), R150 (R150*), Q152 (Q152*), Q177 (Q177*), Y190 (Y190*), Y228 (Y228*), E243 (E243*), Q255 (Q255*), Q264 (Q264*), Y302 (Y302*), R335 (R335*), Q346 (Q346*), E353 (E353*), Q383 (Q383*), Q449 (Q449*), E463 (E463*), Y523 (Y523*), W526 (W526*), R598 (R598*), Q610 (Q610*), Q641 (Q641*), E648 (E648*), Q699 (Q699*), Q706 (Q706*), Q765 (Q765*), Q771 (Q771*), E806 (E806*), Y827 (Y827*), or any combination thereof, wherein the numbering of the residues is according to SEQ ID NO: 50, or according to the amino acid sequence of a human CDH1 polypeptide. In some embodiments, the one or more deleterious CDH1 mutations comprise a frameshift, wherein the frameshift is an T115fs*53, P127fs*41, P200fs*6, P200fs*16, V202fs*7, H233fs*11, P277fs*5, Y302fs*1, S337fs*12, Y523fs*1, L585fs*4, I650fs*3, I650fs*13, A719fs*29, or Q765fs*4 frameshift, wherein the numbering of the residues is according to SEQ ID NO: 50, or according to the amino acid sequence of a human CDH1 polypeptide. In some embodiments, the T115fs*53 frameshift results from a 343_344 insA alteration in a CDH1 nucleic acid molecule, e.g., a CDH1 nucleic acid molecule comprising a sequence corresponding to Transcript ID NM_004360.5, provided herein as SEQ ID NO: 49. In some embodiments, the P127fs*41 frameshift results from a 377_378 insC alteration in a CDH1 nucleic acid molecule, e.g., a CDH1 nucleic acid molecule comprising a sequence corresponding to Transcript ID NM_004360.5, provided herein as SEQ ID NO: 49. In some embodiments, the P200fs*6 frameshift results from a 598_605 delCCCCCTGT alteration in a CDH1 nucleic acid molecule, e.g., a CDH1 nucleic acid molecule comprising a sequence corresponding to Transcript ID NM_004360.5, provided herein as SEQ ID NO: 49. In some embodiments, the P200fs*16 frameshift results from a 598_599 insAC alteration in a CDH1 nucleic acid molecule, e.g., a CDH1 nucleic acid molecule comprising a sequence corresponding to Transcript ID NM_004360.5, provided herein as SEQ ID NO: 49. In some embodiments, the V202fs*7 frameshift results from a 603_604 insT alteration in a CDH1 nucleic acid molecule, e.g., a CDH1 nucleic acid molecule comprising a sequence corresponding to Transcript ID NM_004360.5, provided herein as SEQ ID NO: 49. In some embodiments, the H233fs*11 frameshift results from a 698_699 insA alteration in a CDH1 nucleic acid molecule, e.g., a CDH1 nucleic acid molecule comprising a sequence corresponding to Transcript ID NM_004360.5, provided herein as SEQ ID NO: 49. In some embodiments, the P277fs*5 frameshift results from a 828delT alteration in a CDH1 nucleic acid molecule, e.g., a CDH1 nucleic acid molecule comprising a sequence corresponding to Transcript ID NM_004360.5, provided herein as SEQ ID NO: 49. In some embodiments, the Y302fs*1 frameshift results from a 906delC alteration in a CDH1 nucleic acid molecule, e.g., a CDH1 nucleic acid molecule comprising a sequence corresponding to Transcript ID NM_004360.5, provided herein as SEQ ID NO: 49. In some embodiments, the S337fs*12 frameshift results from a 1009_1010 delAG alteration in a CDH1 nucleic acid molecule, e.g., a CDH1 nucleic acid molecule comprising a sequence corresponding to Transcript ID NM_004360.5, provided herein as SEQ ID NO: 49. In some embodiments, the Y523fs*1 frameshift results from a 1568_1569 insA alteration in a CDH1 nucleic acid molecule, e.g., a CDH1 nucleic acid molecule comprising a sequence corresponding to Transcript ID NM_004360.5, provided herein as SEQ ID NO: 49. In some embodiments, the L585fs*4 frameshift results from a 1753delC alteration in a CDH1 nucleic acid molecule, e.g., a CDH1 nucleic acid molecule comprising a sequence corresponding to Transcript ID NM_004360.5, provided herein as SEQ ID NO: 49. In some embodiments, the I650fs*3 frameshift results from a 1947_1948 insT alteration in a CDH1 nucleic acid molecule, e.g., a CDH1 nucleic acid molecule comprising a sequence corresponding to Transcript ID NM_004360.5, provided herein as SEQ ID NO: 49. In some embodiments, the I650fs*13 frameshift results from a 1947_1948 insT alteration in a CDH1 nucleic acid molecule, e.g., a CDH1 nucleic acid molecule comprising a sequence corresponding to Transcript ID NM_004360.5, provided herein as SEQ ID NO: 49. In some embodiments, the A719fs*29 frameshift results from a 2155_2156 insG alteration in a CDH1 nucleic acid molecule, e.g., a CDH1 nucleic acid molecule comprising a sequence corresponding to Transcript ID NM_004360.5, provided herein as SEQ ID NO: 49. In some embodiments, the Q765fs*4 frameshift results from a 2293_2294 insC alteration in a CDH1 nucleic acid molecule, e.g., a CDH1 nucleic acid molecule comprising a sequence corresponding to Transcript ID NM_004360.5, provided herein as SEQ ID NO: 49. In some embodiments, the one or more deleterious CDH1 mutations comprise a splice site mutation, wherein the splice site mutation is a 48+1G>A, 1565+1G>A, or 1565+1G>T splice site mutation, wherein the numbering of the nucleotides is according to SEQ ID NO: 49, or according the nucleotide sequence of transcript NM_004360.5.

In some embodiments, an ILC metastasis is a bone ILC metastasis, a female reproductive system ILC metastasis, a gastrointestinal ILC metastasis, a liver ILC metastasis, or a skin ILC metastasis. In some embodiments, an ILC metastasis of the disclosure, such as a bone ILC metastasis, a female reproductive system ILC metastasis, a gastrointestinal ILC metastasis, a liver ILC metastasis, or a skin ILC metastasis, comprises one or more biomarkers, e.g., a high tumor mutational burden (e.g., of at least about 10 mut/Mb), a PD-L1-positive status, and/or an alteration in one or more genes. In some embodiments, the one or more genes are selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D. In some embodiments, among a plurality of individuals having an ILC metastasis, at least about 20%, at least about 21%, at least about 25%, at least about 30%, at least about 35%, at least about 40%, at least about 45%, at least about 50%, at least about 55%, at least about 60%, or at least about 65% of the individuals have an ILC metastasis comprising a high tumor mutational burden (e.g., of at least about 10 mut/Mb). In some embodiments, among a plurality of individuals having an ILC metastasis, at least about 15%, at least about 17%, at least about 20%, at least about 21%, at least about 25%, at least about 30%, at least about 35%, at least about 40%, at least about 45%, at least about 50%, at least about 55%, at least about 60%, or at least about 65% of the individuals have a PD-L1-positive ILC metastasis. In some embodiments, among a plurality of individuals having an ILC metastasis, at least about 55%, at least about 57%, at least about 60%, or at least about 65% of the individuals have an ILC metastasis comprising an alteration in PIK3CA. In some embodiments, among a plurality of individuals having an ILC metastasis, at least about 4%, at least about 10%, at least about 15%, at least about 20%, at least about 25%, at least about 30%, at least about 35%, at least about 40%, at least about 45%, at least about 50%, at least about 55%, at least about 60%, or at least about 65% of the individuals have an ILC metastasis comprising an alteration in BRCA1 and/or BRCA2. In some embodiments, among a plurality of individuals having an ILC metastasis, at least about 15%, at least about 18%, at least about 20%, at least about 25%, at least about 30%, at least about 35%, at least about 40%, at least about 45%, at least about 50%, at least about 55%, at least about 60%, or at least about 65% of the individuals have an ILC metastasis comprising an alteration in ESR1. In some embodiments, among a plurality of individuals having an ILC metastasis, at least about 10%, at least about 12%, at least about 15%, at least about 20%, at least about 25%, at least about 30%, at least about 35%, at least about 40%, at least about 45%, at least about 50%, at least about 55%, at least about 60%, or at least about 65% of the individuals have an ILC metastasis comprising an alteration in ERBB2. In some embodiments, among a plurality of individuals having an ILC metastasis, at least about 10%, at least about 12%, at least about 15%, at least about 20%, at least about 25%, at least about 30%, at least about 35%, at least about 40%, at least about 45%, at least about 50%, at least about 55%, at least about 60%, or at least about 65% of the individuals have an ILC metastasis comprising an alteration in ARID1A. In some embodiments, among a plurality of individuals having an ILC metastasis, at least about 5%, at least about 8%, at least about 10%, at least about 15%, at least about 20%, at least about 25%, at least about 30%, at least about 35%, at least about 40%, at least about 45%, at least about 50%, at least about 55%, at least about 60%, or at least about 65% of the individuals have an ILC metastasis comprising an alteration in NF 1. In some embodiments, among a plurality of individuals having an ILC metastasis, at least about 4%, at least about 6%, at least about 10%, at least about 15%, at least about 20%, at least about 25%, at least about 30%, at least about 35%, at least about 40%, at least about 45%, at least about 50%, at least about 55%, at least about 60%, or at least about 65% of the individuals have an ILC metastasis comprising an alteration in RB1. In some embodiments, among a plurality of individuals having an ILC metastasis, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, or at least about 65% of the individuals have an ILC metastasis comprising an alteration in KRAS. In some embodiments, among a plurality of individuals having an ILC metastasis, at least about 4%, at least about 10%, at least about 15%, at least about 20%, at least about 25%, at least about 30%, at least about 35%, at least about 40%, at least about 45%, at least about 50%, at least about 55%, at least about 60%, or at least about 65% of the individuals have an ILC metastasis comprising an alteration in PTEN.

In some embodiments, among a plurality of individuals having a gastrointestinal ILC metastasis, at least about 20%, at least about 23%, at least about 25%, at least about 30%, at least about 35%, at least about 40%, at least about 45%, at least about 50%, at least about 55%, at least about 60%, or at least about 65% of the individuals have a gastrointestinal ILC metastasis comprising a high tumor mutational burden (e.g., of at least about 10 mut/Mb). In some embodiments, among a plurality of individuals having a gastrointestinal ILC metastasis, at least about 45%, at least about 47%, at least about 50%, at least about 55%, at least about 60%, or at least about 65% of the individuals have a PD-L1-positive gastrointestinal ILC metastasis. In some embodiments, among a plurality of individuals having a gastrointestinal ILC metastasis, at least about 55%, at least about 59%, at least about 60%, or at least about 65% of the individuals have a gastrointestinal ILC metastasis comprising an alteration in PIK3CA. In some embodiments, among a plurality of individuals having a gastrointestinal ILC metastasis at least about 15%, at least about 20%, at least about 25%, at least about 30%, at least about 35%, at least about 40%, at least about 45%, at least about 50%, at least about 55%, at least about 60%, or at least about 65% of the individuals have a gastrointestinal ILC metastasis comprising an alteration in ESR1. In some embodiments, among a plurality of individuals having a gastrointestinal ILC metastasis, at least about 10%, at least about 14%, at least about 20%, at least about 25%, at least about 30%, at least about 35%, at least about 40%, at least about 45%, at least about 50%, at least about 55%, at least about 60%, or at least about 65% of the individuals have a gastrointestinal ILC metastasis comprising an alteration in ARID1A. In some embodiments, among a plurality of individuals having a gastrointestinal ILC metastasis, at least about 4%, at least about 5%, at least about 6%, at least about 10%, at least about 15%, at least about 20%, at least about 25%, at least about 30%, at least about 35%, at least about 40%, at least about 45%, at least about 50%, at least about 55%, at least about 60%, or at least about 65% of the individuals have a gastrointestinal ILC metastasis comprising an alteration in NF1. In some embodiments, among a plurality of individuals having a gastrointestinal ILC metastasis, at least about 5%, at least about 7%, at least about 10%, at least about 15%, at least about 20%, at least about 25%, at least about 30%, at least about 35%, at least about 40%, at least about 45%, at least about 50%, at least about 55%, at least about 60%, or at least about 65% of the individuals have a gastrointestinal ILC metastasis comprising an alteration in RB1. In some embodiments, among a plurality of individuals having a gastrointestinal ILC metastasis, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, or at least about 65% of the individuals have a gastrointestinal ILC metastasis comprising an alteration in KRAS. In some embodiments, among a plurality of individuals having a gastrointestinal ILC metastasis, at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, or at least about 65% of the individuals have a gastrointestinal ILC metastasis comprising an alteration in TERT.

In some embodiments, among a plurality of individuals having a liver ILC metastasis, at least about 15%, at least about 18%, at least about 20%, at least about 25%, at least about 30%, at least about 35%, at least about 40%, at least about 45%, at least about 50%, at least about 55%, at least about 60%, or at least about 65% of the individuals have a liver ILC metastasis comprising a high tumor mutational burden (e.g., of at least about 10 mut/Mb). In some embodiments, among a plurality of individuals having a liver ILC metastasis, at least about 10%, at least about 11%, at least about 15%, at least about 18%, at least about 20%, at least about 25%, at least about 30%, at least about 35%, at least about 40%, at least about 45%, at least about 50%, at least about 55%, at least about 60%, or at least about 65% of the individuals have a PD-L1-positive liver ILC metastasis. In some embodiments, among a plurality of individuals having a liver ILC metastasis, at least about 55%, at least about 57%, at least about 60%, or at least about 65% of the individuals have a liver ILC metastasis comprising an alteration in PIK3CA. In some embodiments, among a plurality of individuals having a liver ILC metastasis, at least about 4%, at least about 10%, at least about 15%, at least about 20%, at least about 25%, at least about 30%, at least about 35%, at least about 40%, at least about 45%, at least about 50%, at least about 55%, at least about 60%, or at least about 65% of the individuals have a liver ILC metastasis comprising an alteration in BRCA1 and/or BRCA2. In some embodiments, among a plurality of individuals having a liver ILC metastasis, at least about 20%, at least about 25%, at least about 30%, at least about 35%, at least about 40%, at least about 45%, at least about 50%, at least about 55%, at least about 60%, or at least about 65% of the individuals have a liver ILC metastasis comprising an alteration in ESR1. In some embodiments, among a plurality of individuals having a liver ILC metastasis, at least about 15%, at least about 20%, at least about 25%, at least about 30%, at least about 35%, at least about 40%, at least about 45%, at least about 50%, at least about 55%, at least about 60%, or at least about 65% of the individuals have a liver ILC metastasis comprising an alteration in ERBB2. In some embodiments, among a plurality of individuals having a liver ILC metastasis, at least about 10%, at least about 11%, at least about 15%, at least about 20%, at least about 25%, at least about 30%, at least about 35%, at least about 40%, at least about 45%, at least about 50%, at least about 55%, at least about 60%, or at least about 65% of the individuals have a liver ILC metastasis comprising an alteration in ARID1A. In some embodiments, among a plurality of individuals having a liver ILC metastasis, at least about 5%, at least about 8%, at least about 10%, at least about 15%, at least about 20%, at least about 25%, at least about 30%, at least about 35%, at least about 40%, at least about 45%, at least about 50%, at least about 55%, at least about 60%, or at least about 65% of the individuals have a liver ILC metastasis comprising an alteration in NF1. In some embodiments, among a plurality of individuals having a liver ILC metastasis, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, or at least about 65% of the individuals have a liver ILC metastasis comprising an alteration in RB1. In some embodiments, among a plurality of individuals having a liver ILC metastasis, at least about 5%, at least about 6%, at least about 10%, at least about 15%, at least about 20%, at least about 25%, at least about 30%, at least about 35%, at least about 40%, at least about 45%, at least about 50%, at least about 55%, at least about 60%, or at least about 65% of the individuals have a liver ILC metastasis comprising an alteration in KRAS. In some embodiments, among a plurality of individuals having a liver ILC metastasis, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, or at least about 65% of the individuals have a liver ILC metastasis comprising an alteration in PTEN. In some embodiments, among a plurality of individuals having a liver ILC metastasis, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, or at least about 65% of the individuals have a liver ILC metastasis comprising an alteration in FGFR2. In some embodiments, among a plurality of individuals having a liver ILC metastasis, at least about 4%, at least about 10%, at least about 15%, at least about 20%, at least about 25%, at least about 30%, at least about 35%, at least about 40%, at least about 45%, at least about 50%, at least about 55%, at least about 60%, or at least about 65% of the individuals have a liver ILC metastasis comprising an alteration in SMAD4.

In some embodiments, among a plurality of individuals having a female reproductive system ILC metastasis, at least about 10%, at least about 14%, at least about 15%, at least about 20%, at least about 25%, at least about 30%, at least about 35%, at least about 40%, at least about 45%, at least about 50%, at least about 55%, at least about 60%, or at least about 65% of the individuals have a female reproductive system ILC metastasis comprising a high tumor mutational burden (e.g., of at least about 10 mut/Mb). In some embodiments, among a plurality of individuals having a female reproductive system ILC metastasis, at least about 15%, at least about 18%, at least about 20%, at least about 25%, at least about 30%, at least about 35%, at least about 40%, at least about 45%, at least about 50%, at least about 55%, at least about 60%, or at least about 65% of the individuals have a PD-L1-positive female reproductive system ILC metastasis. In some embodiments, among a plurality of individuals having a female reproductive system ILC metastasis, at least about 50%, at least about 55%, at least about 60%, at least about 62%, or at least about 65% of the individuals have a female reproductive system ILC metastasis comprising an alteration in PIK3CA. In some embodiments, among a plurality of individuals having a female reproductive system ILC metastasis, at least about 5%, at least about 7%, at least about 10%, at least about 15%, at least about 20%, at least about 25%, at least about 30%, at least about 35%, at least about 40%, at least about 45%, at least about 50%, at least about 55%, at least about 60%, or at least about 65% of the individuals have a female reproductive system ILC metastasis comprising an alteration in BRCA1 and/or BRCA2. In some embodiments, among a plurality of individuals having a female reproductive system ILC metastasis, at least about 5%, at least about 7%, at least about 10%, at least about 15%, at least about 20%, at least about 25%, at least about 30%, at least about 35%, at least about 40%, at least about 45%, at least about 50%, at least about 55%, at least about 60%, or at least about 65% of the individuals have a female reproductive system ILC metastasis comprising an alteration in ARID1A. In some embodiments, among a plurality of individuals having a female reproductive system ILC metastasis, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, or at least about 65% of the individuals have a female reproductive system ILC metastasis comprising an alteration in NF1. In some embodiments, among a plurality of individuals having a female reproductive system ILC metastasis, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, or at least about 65% of the individuals have a female reproductive system ILC metastasis comprising an alteration in PTEN. In some embodiments, among a plurality of individuals having a female reproductive system ILC metastasis, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, or at least about 65% of the individuals have a female reproductive system ILC metastasis comprising an alteration in NCOR1.

In some embodiments, among a plurality of individuals having a bone ILC metastasis, at least about 15%, at least about 19%, at least about 20%, at least about 25%, at least about 30%, at least about 35%, at least about 40%, at least about 45%, at least about 50%, at least about 55%, at least about 60%, or at least about 65% of the individuals have a bone ILC metastasis comprising a high tumor mutational burden (e.g., of at least about 10 mut/Mb). In some embodiments, among a plurality of individuals having a bone ILC metastasis, at least about 55%, at least about 60%, or at least about 65% of the individuals have a bone ILC metastasis comprising an alteration in PIK3CA. In some embodiments, among a plurality of individuals having a bone ILC metastasis, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, or at least about 65% of the individuals have a bone ILC metastasis comprising an alteration in BRCA1 and/or BRCA2. In some embodiments, among a plurality of individuals having a bone ILC metastasis, at least about 10%, at least about 11%, at least about 15%, at least about 20%, at least about 25%, at least about 30%, at least about 35%, at least about 40%, at least about 45%, at least about 50%, at least about 55%, at least about 60%, or at least about 65% of the individuals have a bone ILC metastasis comprising an alteration in ESR1. In some embodiments, among a plurality of individuals having a bone ILC metastasis, at least about 5%, at least about 9%, at least about 10%, at least about 15%, at least about 20%, at least about 25%, at least about 30%, at least about 35%, at least about 40%, at least about 45%, at least about 50%, at least about 55%, at least about 60%, or at least about 65% of the individuals have a bone ILC metastasis comprising an alteration in ERBB2. In some embodiments, among a plurality of individuals having a bone ILC metastasis, at least about 5%, at least about 9%, at least about 10%, at least about 15%, at least about 20%, at least about 25%, at least about 30%, at least about 35%, at least about 40%, at least about 45%, at least about 50%, at least about 55%, at least about 60%, or at least about 65% of the individuals have a bone ILC metastasis comprising an alteration in ARID1A. In some embodiments, among a plurality of individuals having a bone ILC metastasis, at least about 5%, at least about 10%, at least about 15%, at least about 20%, at least about 25%, at least about 30%, at least about 35%, at least about 40%, at least about 45%, at least about 50%, at least about 55%, at least about 60%, or at least about 65% of the individuals have a bone ILC metastasis comprising an alteration in NF1. In some embodiments, among a plurality of individuals having a bone ILC metastasis, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, or at least about 65% of the individuals have a bone ILC metastasis comprising an alteration in RB1. In some embodiments, among a plurality of individuals having a bone ILC metastasis, at least about 5%, at least about 6%, at least about 10%, at least about 15%, at least about 20%, at least about 25%, at least about 30%, at least about 35%, at least about 40%, at least about 45%, at least about 50%, at least about 55%, at least about 60%, or at least about 65% of the individuals have a bone ILC metastasis comprising an alteration in KRAS. In some embodiments, among a plurality of individuals having a bone ILC metastasis, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, or at least about 65% of the individuals have a bone ILC metastasis comprising an alteration in FGFR2. In some embodiments, among a plurality of individuals having a bone ILC metastasis, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, or at least about 65% of the individuals have a bone ILC metastasis comprising an alteration in BRAF.

In some embodiments, among a plurality of individuals having a skin ILC metastasis, at least about 15%, at least about 20%, at least about 21%, at least about 25%, at least about 30%, at least about 35%, at least about 40%, at least about 45%, at least about 50%, at least about 55%, at least about 60%, or at least about 65% of the individuals have a skin ILC metastasis comprising a high tumor mutational burden (e.g., of at least about 10 mut/Mb). In some embodiments, among a plurality of individuals having a skin ILC metastasis, at least about 25%, at least about 28%, at least about 30%, at least about 35%, at least about 40%, at least about 45%, at least about 50%, at least about 55%, at least about 60%, or at least about 65% of the individuals have a PD-L1-positive skin ILC metastasis. In some embodiments, among a plurality of individuals having a skin ILC metastasis, at least about 45%, at least about 48%, at least about 50%, at least about 55%, at least about 60%, or at least about 65% of the individuals have a skin ILC metastasis comprising an alteration in PIK3CA. In some embodiments, among a plurality of individuals having a skin ILC metastasis, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, or at least about 65% of the individuals have a skin ILC metastasis comprising an alteration in BRCA1 and/or BRCA2. In some embodiments, among a plurality of individuals having a skin ILC metastasis, at least about 5%, at least about 7%, at least about 10%, at least about 15%, at least about 20%, at least about 25%, at least about 30%, at least about 35%, at least about 40%, at least about 45%, at least about 50%, at least about 55%, at least about 60%, or at least about 65% of the individuals have a skin ILC metastasis comprising an alteration in ESR1. In some embodiments, among a plurality of individuals having a skin ILC metastasis, at least about 5%, at least about 10%, at least about 11%, at least about 15%, at least about 20%, at least about 25%, at least about 30%, at least about 35%, at least about 40%, at least about 45%, at least about 50%, at least about 55%, at least about 60%, or at least about 65% of the individuals have a skin ILC metastasis comprising an alteration in ERBB2. In some embodiments, among a plurality of individuals having a skin ILC metastasis, at least about 5%, at least about 10%, at least about 15%, at least about 20%, at least about 25%, at least about 30%, at least about 35%, at least about 40%, at least about 45%, at least about 50%, at least about 55%, at least about 60%, or at least about 65% of the individuals have a skin ILC metastasis comprising an alteration in ARID1A. In some embodiments, among a plurality of individuals having a skin ILC metastasis, at least about 5%, at least about 8%, at least about 10%, at least about 15%, at least about 20%, at least about 25%, at least about 30%, at least about 35%, at least about 40%, at least about 45%, at least about 50%, at least about 55%, at least about 60%, or at least about 65% of the individuals have a skin ILC metastasis comprising an alteration in NF1. In some embodiments, among a plurality of individuals having a skin ILC metastasis, at least about 5%, at least about 10%, at least about 11%, at least about 15%, at least about 20%, at least about 25%, at least about 30%, at least about 35%, at least about 40%, at least about 45%, at least about 50%, at least about 55%, at least about 60%, or at least about 65% of the individuals have a skin ILC metastasis comprising an alteration in RB1. In some embodiments, among a plurality of individuals having a skin ILC metastasis, at least about 5%, at least about 8%, at least about 10%, at least about 15%, at least about 20%, at least about 25%, at least about 30%, at least about 35%, at least about 40%, at least about 45%, at least about 50%, at least about 55%, at least about 60%, or at least about 65% of the individuals have a skin ILC metastasis comprising an alteration in PTEN.

Biomarkers

Tumor Mutational Burden

In some embodiments, the methods provided herein comprise acquiring knowledge that an ILC metastasis has a high tumor mutational burden. In some embodiments, acquiring knowledge that an ILC metastasis has a high tumor mutational burden comprises measuring the level of tumor mutational burden in a sample, e.g., in a sample from an ILC tumor, obtained from an individual. In some embodiments, the methods provided herein comprise detecting a high tumor mutational burden in a sample from an individual having an ILC metastasis. In some embodiments, the methods comprise administering an effective amount of an immune checkpoint inhibitor responsive to knowledge that the ILC metastasis has a high tumor mutational burden, e.g., of at least about 10 mut/Mb. In some embodiments, the methods comprise providing a report to a party.

In some embodiments, tumor mutational burden is assessed in sample from an individual, such as sample described herein. In some embodiments, the sample from the individual comprises fluid, cells, or tissue. In some embodiments, the sample from the individual comprises a tumor biopsy or a circulating tumor cell. In some embodiments, the sample from the individual comprises nucleic acids. In some embodiments, the sample from the individual comprises mRNA, genomic DNA, circulating tumor DNA, cell-free DNA, or cell-free RNA.

In some embodiments, tumor mutational burden is measured using any suitable method known in the art. For example, tumor mutational burden may be measured using whole-exome sequencing (WES), next-generation sequencing, whole genome sequencing, gene-targeted sequencing, or sequencing of a panel of genes, e.g., panels including cancer-related genes. See, e.g., Melendez et al., Transl Lung Cancer Res (2018) 7(6):661-667. In some embodiments, tumor mutational burden is measured using gene-targeted sequencing, e.g., using a nucleic acid hybridization-capture method, e.g., coupled with sequencing. See, e.g., Fancello et al., J Immunother Cancer (2019) 7:183.

In some embodiments, tumor mutational burden is measured according to the methods provided in WO2017151524A1, which is hereby incorporated by reference in its entirety.

In some embodiments, tumor mutational burden is measured in the sample by whole exome sequencing. In some embodiments, tumor mutational burden is measured in the sample using next-generation sequencing. In some embodiments, tumor mutational burden is measured in the sample using whole genome sequencing. In some embodiments, tumor mutational burden is measured in the sample by gene-targeted sequencing. In some embodiments, tumor mutational burden is measured on between about 0.8 Mb and about 1.1 Mb. In some embodiments, tumor mutational burden is measured on any of about 0.8 Mb, about 0.81 Mb, about 0.82 Mb, about 0.83 Mb, about 0.84 Mb, about 0.85 Mb, about 0.86 Mb, about 0.87 Mb, about 0.88 Mb, about 0.89 Mb, about 0.9 Mb, about 0.91 Mb, about 0.92 Mb, about 0.93 Mb, about 0.94 Mb, about 0.95 Mb, about 0.96 Mb, about 0.97 Mb, about 0.98 Mb, about 0.99 Mb, about 1 Mb, about 1.01 Mb, about 1.02 Mb, about 1.03 Mb, about 1.04 Mb, about 1.05 Mb, about 1.06 Mb, about 1.07 Mb, about 1.08 Mb, about 1.09 Mb, or about 1.1 Mb.

In some embodiments, the ILC metastasis has a high tumor mutational burden, e.g., of at least about 10 mut/Mb. In some embodiments, the ILC metastasis has a tumor mutational burden of at least about 10 mut/Mb. In some embodiments, the ILC metastasis has a tumor mutational burden of any of between about 10 mut/Mb and about 15 mut/Mb, between about 15 mut/Mb and about 20 mut/Mb, between about 20 mut/Mb and about 25 mut/Mb, between about 25 mut/Mb and about 30 mut/Mb, between about 30 mut/Mb and about 35 mut/Mb, between about 35 mut/Mb and about 40 mut/Mb, between about 40 mut/Mb and about 45 mut/Mb, between about 45 mut/Mb and about 50 mut/Mb, between about 50 mut/Mb and about 55 mut/Mb, between about 55 mut/Mb and about 60 mut/Mb, between about 60 mut/Mb and about 65 mut/Mb, between about 65 mut/Mb and about 70 mut/Mb, between about 70 mut/Mb and about 75 mut/Mb, between about 75 mut/Mb and about 80 mut/Mb, between about 80 mut/Mb and about 85 mut/Mb, between about 85 mut/Mb and about 90 mut/Mb, between about 90 mut/Mb and about 95 mut/Mb, or between about 95 mut/Mb and about 100 mut/Mb. In some embodiments, the ILC metastasis has a tumor mutational burden of any of between about 100 mut/Mb and about 110 mut/Mb, between about 110 mut/Mb and about 120 mut/Mb, between about 120 mut/Mb and about 130 mut/Mb, between about 130 mut/Mb and about 140 mut/Mb, between about 140 mut/Mb and about 150 mut/Mb, between about 150 mut/Mb and about 160 mut/Mb, between about 160 mut/Mb and about 170 mut/Mb, between about 170 mut/Mb and about 180 mut/Mb, between about 180 mut/Mb and about 190 mut/Mb, between about 190 mut/Mb and about 200 mut/Mb, between about 210 mut/Mb and about 220 mut/Mb, between about 220 mut/Mb and about 230 mut/Mb, between about 230 mut/Mb and about 240 mut/Mb, between about 240 mut/Mb and about 250 mut/Mb, between about 250 mut/Mb and about 260 mut/Mb, between about 260 mut/Mb and about 270 mut/Mb, between about 270 mut/Mb and about 280 mut/Mb, between about 280 mut/Mb and about 290 mut/Mb, between about 290 mut/Mb and about 300 mut/Mb, between about 300 mut/Mb and about 310 mut/Mb, between about 310 mut/Mb and about 320 mut/Mb, between about 320 mut/Mb and about 330 mut/Mb, between about 330 mut/Mb and about 340 mut/Mb, between about 340 mut/Mb and about 350 mut/Mb, between about 350 mut/Mb and about 360 mut/Mb, between about 360 mut/Mb and about 370 mut/Mb, between about 370 mut/Mb and about 380 mut/Mb, between about 380 mut/Mb and about 390 mut/Mb, between about 390 mut/Mb and about 400 mut/Mb, or more than 400 mut/Mb.

In some embodiments, measuring tumor mutational burden comprises assessing mutations in a sample derived from a cancer in an individual, e.g., an ILC metastasis described herein. In some embodiments, measuring tumor mutational burden comprises assessing mutations in a sample derived from a cancer in an individual, e.g., an ILC metastasis described herein, and in a matched normal sample, e.g., a sample from the individual derived from a tissue or other source that is free of the cancer.

In some embodiments, the sample is obtained or derived from an ILC metastasis, such as a bone ILC metastasis, a female reproductive system ILC metastasis, a gastrointestinal ILC metastasis, a liver ILC metastasis, or a skin ILC metastasis. In some embodiments, the sample is obtained or derived from a gastrointestinal ILC metastasis or a skin ILC metastasis.

PD-L1 Expression

In some embodiments, the methods provided herein comprise acquiring knowledge that an ILC metastasis is PD-L1-positive in a sample from an individual. In some embodiments, acquiring knowledge that the ILC metastasis is PD-L1-positive comprises measuring the level of PD-L1 expression in a sample, e.g., in a sample from an ILC tumor, obtained from an individual. In some embodiments, the methods provided herein comprise detecting a PD-L1-positive ILC metastasis in a sample from an individual. In some embodiments, detecting a PD-L1-positive ILC metastasis in a sample from an individual comprises measuring the level of PD-L1 expression in a sample, e.g., in a sample from an ILC tumor obtained from the individual. In some embodiments, the methods comprise administering an effective amount of an immune checkpoint inhibitor responsive to knowledge that the ILC metastasis is PD-L1-positive. In some embodiments, the methods comprise providing a report to a party.

Any suitable method for measuring PD-L1 expression in a sample from an individual may be used. For example, the level of PD-L1 expression may be measured using immunohistochemistry (IHC), Western blot analysis, immunoprecipitation, molecular binding assays, enzyme-linked immunosorbent assay (ELISA), enzyme-linked immunofiltration assay (ELIFA), fluorescence activated cell sorting (FACS), MassARRAY, proteomics (e.g., mass spectrometry), quantitative blood based assays (as for example serum ELISA), biochemical enzymatic activity assays, in situ hybridization, Northern analysis, polymerase chain reaction (“PCR”) including quantitative real time PCR (qRT-PCR) and other amplification-based methods, RNA-sequencing (RNA-seq), FISH, microarray analysis, gene expression profiling, and/or serial analysis of gene expression (“SAGE”). Multiplexed immunoassays such as those available from Rules Based Medicine or Meso Scale Discovery (“MSD”) may also be used.

In some embodiments, PD-L1 expression in a sample from an individual is measured based on the level of PD-L1 mRNA in the sample. Any suitable method for measuring mRNA expression in a sample from an individual may be used. For example, the level of PD-L1 mRNA expression may be measured using in situ hybridization, Northern analysis, polymerase chain reaction (“PCR”) including quantitative real time PCR (qRT-PCR) and other amplification-based methods, RNA-sequencing (RNA-seq), FISH, microarray analysis, gene expression profiling, and/or serial analysis of gene expression (“SAGE”).

In some embodiments, PD-L1 expression in a sample from an individual is measured based on the level of PD-L1 protein in the sample. Any suitable method for measuring protein expression in a sample from an individual may be used. For example, the level of PD-L1 protein expression may be measured using immunohistochemistry (IHC), Western blot analysis, immunoprecipitation, molecular binding assays, enzyme-linked immunosorbent assay (ELISA), enzyme-linked immunofiltration assay (ELIFA), fluorescence activated cell sorting (FACS), proteomics (e.g., mass spectrometry), quantitative blood based assays (as for example serum ELISA), biochemical enzymatic activity assays, or multiplexed immunoassays such as those available from Rules Based Medicine or Meso Scale Discovery (“MSD”).

In some embodiments, the level of PD-L1 protein expression is measured using an immunohistochemistry assay. In some embodiments, the level of PD-L1 protein expression is measured using a VENTANA PD-L1 assay (SP142). In some embodiments, the level of PD-L1 protein expression is determined based on PD-L1 expression in tumor infiltrating immune cells (ICs) and/or tumor cells (TCs). Additional information about the VENTANA SP142 assay may be found in the website: www[dot]accessdata[dot]fda[dot]gov/cdrh_docs/pdf16/P160002c.pdf.

In some embodiments, an ILC metastasis provided herein is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of tumor infiltrating immune cells (ICs) and/or tumor cells (TCs) express PD-L1 protein and/or PD-L1 mRNA (e.g., are positive for PD-L1 protein and/or PD-L1 mRNA). In some embodiments, an ILC metastasis provided herein is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of tumor infiltrating immune cells (ICs) express PD-L1 protein and/or PD-L1 mRNA (e.g., are positive for PD-L1 protein and/or PD-L1 mRNA).

In some embodiments, an ILC metastasis provided herein is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of tumor infiltrating immune cells (ICs) and/or tumor cells (TCs) in a sample from an individual express PD-L1 protein and/or PD-L1 mRNA (e.g., are positive for PD-L1 protein and/or PD-L1 mRNA). In some embodiments, an ILC metastasis provided herein is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of tumor infiltrating immune cells (ICs) in a sample from an individual express PD-L1 protein and/or PD-L1 mRNA (e.g., are positive for PD-L1 protein and/or PD-L1 mRNA).

In some embodiments, a sample from an individual is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of tumor infiltrating immune cells (ICs) and/or tumor cells (TCs) in the sample express PD-L1 protein and/or PD-L1 mRNA (e.g., are positive for PD-L1 protein and/or PD-L1 mRNA). In some embodiments, a sample from an individual is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of tumor infiltrating immune cells (ICs) in the sample express PD-L1 protein and/or PD-L1 mRNA (e.g., are positive for PD-L1 protein and/or PD-L1 mRNA).

In some embodiments, the level of PD-L1 protein expression is measured using a PD-L1 IHC assay such as a DAKO 22C3 assay. In some embodiments, the level of PD-L1 protein expression is determined based on a combined positive score (CPS), which is the number of PD-L1 staining cells (e.g., tumor cells, lymphocytes, macrophages or histiocytes) divided by the total number of viable tumor cells, and multiplied by 100. Additional information about the DAKO 22C3 assay may be found in the website: www[dot]agilent[dot]com/cs/library/usermanuals/public/29219pd-11-ihc-22C3-pharmdx-gastric-interpretation-manual_us.pclf. In some embodiments, a sample from an individual is determined to be positive for PD-L1 if it has a combined positive score (CPS) of at least about 10. In some embodiments, a sample from an individual has a combined positive score (CPS) of at least about 10. In some embodiments, a sample from an individual has a combined positive score (CPS) of between about 10 and about 15, between about 15 and about 20, between about 20 and about between about 25 and about 30, between about 30 and about 35, between about 35 and about 40, between about 40 and about 45, between about 45 and about 50, between about 50 and about 55, between about 55 and about 60, between about 60 and about 65, between about 65 and about 70, between about 70 and about 75, between about 75 and about 80, between about 80 and about 85, between about 85 and about 90, between about 90 and about 95, or about 100.

In some embodiments, the level of PD-L1 protein and/or PD-L1 mRNA is assessed in sample from an individual, such as a sample described herein. In some embodiments, the sample from the individual comprises fluid, cells, or tissue. In some embodiments, the sample from the individual comprises a tumor biopsy or a circulating tumor cell. In some embodiments, the sample is obtained or derived from an ILC metastasis, such as a bone ILC metastasis, a female reproductive system ILC metastasis, a gastrointestinal ILC metastasis, a liver ILC metastasis, or a skin ILC metastasis.

Gene Alterations

In some embodiments, the methods provided herein comprise acquiring knowledge that an ILC metastasis comprises an alteration in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D in a sample from an individual having an ILC metastasis. In some embodiments, acquiring knowledge that the ILC metastasis comprises an alteration in one or more genes comprises detecting the alteration in a sample, e.g., in a sample from an ILC tumor, obtained from an individual. In some embodiments, the methods provided herein comprise detecting an alteration in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D in a sample from an individual having an ILC metastasis. In some embodiments, the methods further comprise administering an effective amount of an anti-cancer agent responsive to knowledge of the presence of an alteration in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D in a sample from an individual having an ILC metastasis. In some embodiments, the methods further comprise providing a report to a party.

In some embodiments, the alteration in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D comprises one or more of a substitution of one or more nucleotides, an insertion of one or more nucleotides, or a deletion of one or more nucleotides. In some embodiments, the alteration in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D comprises a gene copy number alteration. In some embodiments, the alteration in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D comprises a gene amplification. In some embodiments, the alteration in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D comprises a gene deletion, e.g., a deletion of the entire gene or of a portion of the gene. In some embodiments, the alteration in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D is a point mutation. In some embodiments, the alteration in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D comprises a single nucleotide polymorphism. In some embodiments, the alteration in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D comprises an alteration in an exon and/or an intron of the gene. In some embodiments, the alteration in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D is a non-synonymous mutation. In some embodiments, the alteration in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D is a missense mutation. In some embodiments, the alteration in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D is a nonsense mutation. In some embodiments, the alteration in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D is a gain-of-function mutation, e.g., an activating mutation. In some embodiments, the alteration in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D is a loss-of-function mutation, e.g., an inactivating mutation. In some embodiments, the alteration in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D results in a frameshift. In some embodiments, the alteration in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D results in a premature stop codon. In some embodiments, the alteration in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D comprises a functional alteration. In some embodiments, the alteration in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D comprises a mutation that alters the function of the polypeptide or protein encoded by the gene. In some embodiments, the alteration in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D comprises a complex insertion. In some embodiments, the alteration in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D comprises a complex deletion. In some embodiments, the alteration in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D is a mutation in a splice site. In some embodiments, the alteration in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D alters the splicing of an mRNA molecule encoded by the gene.

In some embodiments, the alteration in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D comprises an insertion of one or more nucleotides. In some embodiments, the alteration comprises an insertion of between about 1 and about 5 nucleotides, between about 5 and about 10 nucleotides, between about 10 and about 20 nucleotides, between about 20 and about 30 nucleotides, between about 30 and about 40 nucleotides, or between about 40 and about 50 nucleotides. In some embodiments, the alteration comprises an insertion of between about 50 and about 100 nucleotides, between about 100 and about 200 nucleotides, between about 200 and about 300 nucleotides, between about 300 and 400 nucleotides, between about 400 and about 500 nucleotides, between about 500 and about 600 nucleotides, between about 600 and about 700 nucleotides, between about 700 and about 800 nucleotides, between about 800 and about 900 nucleotides, or between about 900 and about 1000 nucleotides. In some embodiments, the alteration comprises an insertion of between about 1000 and about 1500 nucleotides, between about 1500 and about 2000 nucleotides, between about 2000 and about 2500 nucleotides, between about 2500 and about 3000 nucleotides, between about 3000 and about 3500 nucleotides, between about 3500 and about 4000 nucleotides, between about 4000 and about 4500 nucleotides, between about 4500 and about 5000 nucleotides, between about 5000 and about 5500 nucleotides, between about 5500 and about 6000 nucleotides, between about 6000 and about 6500 nucleotides, between about 6500 and about 7000 nucleotides, between about 7000 and about 7500 nucleotides, between about 7500 and about 8000 nucleotides, between about 8000 and about 8500 nucleotides, between about 8500 and about 9000 nucleotides, between about 9000 and about 9500 nucleotides, or between about 9500 and about 10000 nucleotides.

In some embodiments, the alteration in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D comprises a deletion of one or more nucleotides. In some embodiments, the alteration comprises a deletion of between about 1 and about 5 nucleotides, between about 5 and about 10 nucleotides, between about 10 and about 20 nucleotides, between about 20 and about 30 nucleotides, between about 30 and about 40 nucleotides, or between about 40 and about 50 nucleotides. In some embodiments, the alteration comprises a deletion of between about 50 and about 100 nucleotides, between about 100 and about 200 nucleotides, between about 200 and about 300 nucleotides, between about 300 and 400 nucleotides, between about 400 and about 500 nucleotides, between about 500 and about 600 nucleotides, between about 600 and about 700 nucleotides, between about 700 and about 800 nucleotides, between about 800 and about 900 nucleotides, or between about 900 and about 1000 nucleotides. In some embodiments, the alteration comprises a deletion of between about 1000 and about 1500 nucleotides, between about 1500 and about 2000 nucleotides, between about 2000 and about 2500 nucleotides, between about 2500 and about 3000 nucleotides, between about 3000 and about 3500 nucleotides, between about 3500 and about 4000 nucleotides, between about 4000 and about 4500 nucleotides, between about 4500 and about 5000 nucleotides, between about 5000 and about 5500 nucleotides, between about 5500 and about 6000 nucleotides, between about 6000 and about 6500 nucleotides, between about 6500 and about 7000 nucleotides, between about 7000 and about 7500 nucleotides, between about 7500 and about 8000 nucleotides, between about 8000 and about 8500 nucleotides, between about 8500 and about 9000 nucleotides, between about 9000 and about 9500 nucleotides, or between about 9500 and about 10000 nucleotides.

In some embodiments, the alteration in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D results in a substitution, insertion, or deletion of one or more amino acid residues in a polypeptide or a protein encoded by the gene. In some embodiments, the alteration in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D results in a substitution of one or more amino acid residues in a polypeptide or a protein encoded by the gene. In some embodiments, the alteration in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D results in a deletion of one or more amino acid residues in a polypeptide or a protein encoded by the gene. In some embodiments, the alteration in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D results in an insertion of one or more amino acid residues in a polypeptide or a protein encoded by the gene.

In some embodiments, the methods provided herein comprise acquiring knowledge that an ILC metastasis comprises an alteration in PIK3CA in a sample from an individual having an ILC metastasis. An exemplary nucleic acid sequence of PIK3CA is available as Transcript ID NM_006218, provided herein as SEQ ID NO: 2, and available at the website: www[dot]ncbi[dot]nlm[dot]nih[dot]gov/nuccore/NM_006218.

An exemplary amino acid sequence of a PIK3CA polypeptide is provided herein in SEQ ID NO: 26.

(SEQ ID NO: 26)
MPPRPSSGELWGIHLMPPRILVECLLPNGMIVTLECLREATLITIKHELFKEARKYPLHQLLQDESSY
IFVSVTQEAEREEFFDETRRLCDLRLFQPFLKVIEPVGNREEKILNREIGFAIGMPVCEFDMVKDPEV
QDFRRNILNVCKEAVDLRDLNSPHSRAMYVYPPNVESSPELPKHIYNKLDKGQIIVVIWVIVSPNNDK
QKYTLKINHDCVPEQVIAEAIRKKTRSMLLSSEQLKLCVLEYQGKYILKVCGCDEYFLEKYPLSQYKY
IRSCIMLGRMPNLMLMAKESLYSQLPMDCFTMPSYSRRISTATPYMNGETSTKSLWVINSALRIKILC
ATYVNVNIRDIDKIYVRTGIYHGGEPLCDNVNTQRVPCSNPRWNEWLNYDIYIPDLPRAARLCLSICS
VKGRKGAKEEHCPLAWGNINLFDYTDTLVSGKMALNLWPVPHGLEDLLNPIGVTGSNPNKETPCLELE
FDWFSSVVKFPDMSVIEEHANWSVSREAGFSYSHAGLSNRLARDNELRENDKEQLKAISTRDPLSEIT
EQEKDFLWSHRHYCVTIPEILPKLLLSVKWNSRDEVAQMYCLVKDWPPIKPEQAMELLDCNYPDPMVR
GFAVRCLEKYLTDDKLSQYLIQLVQVLKYEQYLDNLLVRFLLKKALTNQRIGHFFFWHLKSEMHNKTV
SQRFGLLLESYCRACGMYLKHLNRQVEAMEKLINLTDILKQEKKDETQKVQMKFLVEQMRRPDEMDAL
QGFLSPLNPAHQLGNLRLEECRIMSSAKRPLWLNWENPDIMSELLFQNNEIIFKNGDDLRQDMLTLQI
IRIMENIWQNQGLDLRMLPYGCLSIGDCVGLIEVVRNSHTIMQIQCKGGLKGALQFNSHTLHQWLKDK
NKGEIYDAAIDLFTRSCAGYCVATFILGIGDRHNSNIMVKDDGQLFHIDEGHELDHKKKKFGYKRERV
PFVLTQDFLIVISKGAQECTKTREFERFQEMCYKAYLAIRQHANLFINLFSMMLGSGMPELQSEDDIA
YIRKTLALDKTEQEALEYFMKQMNDAHHGGWTTKMDWIFHTIKQHALN

In some embodiments, acquiring knowledge that the ILC metastasis comprises an alteration in PIK3CA comprises detecting the alteration in a sample, e.g., in a sample from an ILC tumor, obtained from an individual. In some embodiments, the methods provided herein comprise detecting an alteration in PIK3CA in a sample from an individual having an ILC metastasis. In some embodiments, the methods further comprise administering an effective amount of an anti-cancer agent responsive to knowledge of the presence of an alteration in PIK3CA in a sample from an individual having an ILC metastasis. In some embodiments, the methods further comprise providing a report to a party. In some embodiments, the PIK3CA alteration comprises one or more of a substitution of one or more nucleotides, an insertion of one or more nucleotides, or a deletion of one or more nucleotides in PIK3CA. In some embodiments, the ILC metastasis is a bone ILC metastasis, a female reproductive system ILC metastasis, a gastrointestinal ILC metastasis, a liver ILC metastasis, or a skin ILC metastasis. In some embodiments, the methods provided herein comprise acquiring knowledge that an ILC metastasis comprises an alteration in PIK3CA in a sample from an individual having a bone ILC metastasis. In some embodiments, the methods provided herein comprise acquiring knowledge that an ILC metastasis comprises an alteration in PIK3CA in a sample from an individual having a female reproductive system ILC metastasis. In some embodiments, the methods provided herein comprise acquiring knowledge that an ILC metastasis comprises an alteration in PIK3CA in a sample from an individual having a gastrointestinal ILC metastasis. In some embodiments, the methods provided herein comprise acquiring knowledge that an ILC metastasis comprises an alteration in PIK3CA in a sample from an individual having a liver ILC metastasis. In some embodiments, the methods provided herein comprise acquiring knowledge that an ILC metastasis comprises an alteration in PIK3CA in a sample from an individual having a skin ILC metastasis. In some embodiments, the methods provided herein comprise detecting an alteration in PIK3CA in a sample from an individual having a bone ILC metastasis. In some embodiments, the methods provided herein comprise detecting an alteration in PIK3CA in a sample from an individual having a female reproductive system ILC metastasis. In some embodiments, the methods provided herein comprise detecting an alteration in PIK3CA in a sample from an individual having a gastrointestinal ILC metastasis. In some embodiments, the methods provided herein comprise detecting an alteration in PIK3CA in a sample from an individual having a liver ILC metastasis. In some embodiments, the methods provided herein comprise detecting an alteration in PIK3CA in a sample from an individual having a skin ILC metastasis.

In some embodiments, the alteration in PIK3CA comprises an alteration that results in one or more amino acid substitutions in a polypeptide encoded by the PIK3CA gene. In some embodiments, the one or more amino acid substitutions are at amino acid position E81, R108, K111, G118, N345, D350, E365, E418, C420, E453, P539, E542, E545, Q546, E726, E970, M1004, M1043, N1044, H1047, G1049, or any combination thereof, wherein the numbering of the residues is according to SEQ ID NO: 26 or according to the amino acid sequence of a human PIK3CA protein. In some embodiments, the one or more amino acid substitutions comprise a E81K, R108H, K111N, G118D, N345K, D350N, E365K, E418K, C420R, E453K, E453Q, P539R, E542K, E545K, E545A, E545Q, Q546R, Q546K, E726K, E970K, M1004I, M1043I, N1044K, H1047R, H1047L, or G1049R amino acid substitution, or any combination thereof, wherein the numbering of the residues is according to SEQ ID NO: 26 or according to the amino acid sequence of a human PIK3CA protein. In some embodiments, the alteration in PIK3CA comprises an alteration that results in a deletion of one or more amino acids in a polypeptide encoded by the PIK3CA gene. In some embodiments, the deletion of one or more amino acids in a polypeptide encoded by the PIK3CA gene comprises a deletion of amino acid residue E110 (E110del), wherein the numbering of the residues is according to SEQ ID NO: 26, or according to the amino acid sequence of a human PIK3CA protein.

In some embodiments, the methods provided herein comprise acquiring knowledge that an ILC metastasis comprises an alteration in BRCA1 in a sample from an individual having an ILC metastasis. An exemplary nucleic acid sequence of BRCA1 is available as Transcript ID NM_007294, provided herein as SEQ ID NO: 3, and available at the website: www[dot]ncbi[dot]nlm[dot]nih[dot]gov/nuccore/NM_007294.

An exemplary amino acid sequence of a BRCA1 polypeptide is provided herein in SEQ ID NO: 27.

(SEQ ID NO: 27)
MDLSALRVEEVQNVINAMQKILECPICLELIKEPVSTKCDHIFCKFCMLKLLNQKKGPSQCPLCKNDI
TKRSLQESTRFSQLVEELLKIICAFQLDTGLEYANSYNFAKKENNSPEHLKDEVSIIQSMGYRNRAKR
LLQSEPENPSLQETSLSVQLSNLGTVRTLRTKQRIQPQKTSVYIELGSDSSEDTVNKATYCSVGDQEL
LQITPQGTRDEISLDSAKKAACEFSETDVTNTEHHQPSNNDLNTTEKRAAERHPEKYQGSSVSNLHVE
PCGTNTHASSLQHENSSLLLTKDRMNVEKAEFCNKSKQPGLARSQHNRWAGSKETCNDRRTPSTEKKV
DLNADPLCERKEWNKQKLPCSENPRDTEDVPWITLNSSIQKVNEWFSRSDELLGSDDSHDGESESNAK
VADVLDVLNEVDEYSGSSEKIDLLASDPHEALICKSERVHSKSVESNIEDKIFGKTYRKKASLPNLSH
VTENLIIGAFVTEPQIIQERPLTNKLKRKRRPTSGLHPEDFIKKADLAVQKTPEMINQGTNQTEQNGQ
VMNITNSGHENKTKGDSIQNEKNPNPIESLEKESAFKTKAEPISSSISNMELELNIHNSKAPKKNRLR
RKSSTRHIHALELVVSRNLSPPNCTELQIDSCSSSEEIKKKKYNQMPVRHSRNLQLMEGKEPATGAKK
SNKPNEQTSKRHDSDTFPELKLTNAPGSFTKCSNTSELKEFVNPSLPREEKEEKLETVKVSNNAEDPK
DLMLSGERVLQTERSVESSSISLVPGTDYGTQESISLLEVSTLGKAKTEPNKCVSQCAAFENPKGLIH
GCSKDNRNDTEGFKYPLGHEVNHSRETSIEMEESELDAQYLQNTFKVSKRQSFAPFSNPGNAEEECAT
FSAHSGSLKKQSPKVTFECEQKEENQGKNESNIKPVQTVNITAGFPVVGQKDKPVDNAKCSIKGGSRF
CLSSQFRGNETGLITPNKHGLLQNPYRIPPLFPIKSFVKTKCKKNLLEENFEEHSMSPEREMGNENIP
STVSTISRNNIRENVFKEASSSNINEVGSSTNEVGSSINEIGSSDENIQAELGRNRGPKLNAMLRLGV
LQPEVYKQSLPGSNCKHPEIKKQEYEEVVQTVNTDESPYLISDNLEQPMGSSHASQVCSETPDDLLDD
GEIKEDTSFAENDIKESSAVESKSVQKGELSRSPSPFTHTHLAQGYRRGAKKLESSEENLSSEDEELP
CFQHLLFGKVNNIPSQSTRHSTVATECLSKNTEENLLSLKNSLNDCSNQVILAKASQEHHLSEETKCS
ASLFSSQCSELEDLTANTNTQDPFLIGSSKQMRHQSESQGVGLSDKELVSDDEERGTGLEENNQEEQS
MDSNLGEAASGCESETSVSEDCSGLSSQSDILTTQQRDTMQHNLIKLQQEMAELEAVLEQHGSQPSNS
YPSIISDSSALEDLRNPEQSTSEKAVLTSQKSSEYPISQNPEGLSADKFEVSADSSTSKNKEPGVERS
SPSKCPSLDDRWYMHSCSGSLQNRNYPSQEELIKVVDVEEQQLEESGPHDLTETSYLPRQDLEGTPYL
ESGISLFSDDPESDPSEDRAPESARVGNIPSSTSALKVPQLKVAESAQSPAAAHTTDTAGYNAMEESV
SREKPELTASTERVNKRMSMVVSGLTPEEFMLVYKFARKHHITLTNLITEETTHVVMKTDAEFVCERT
LKYFLGIAGGKWVVSYFWVTQSIKERKMLNEHDFEVRGDVVNGRNHQGPKRARESQDRKIFRGLEICC
YGPFTNMPTDQLEWMVQLCGASVVKELSSFTLGTGVHPIVVVQPDAWTEDNGFHAIGQMCEAPVVTRE
WVLDSVALYQCQELDTYLIPQIPHSHY

In some embodiments, acquiring knowledge that the ILC metastasis comprises an alteration in BRCA1 comprises detecting the alteration in a sample, e.g., in a sample from an ILC tumor, obtained from an individual. In some embodiments, the methods provided herein comprise detecting an alteration in BRCA1 in a sample from an individual having an ILC metastasis. In some embodiments, the methods further comprise administering an effective amount of an anti-cancer agent responsive to knowledge of the presence of an alteration in BRCA1 in a sample from an individual having an ILC metastasis. In some embodiments, the methods further comprise providing a report to a party. In some embodiments, the ILC metastasis is a bone ILC metastasis, a female reproductive system ILC metastasis, a gastrointestinal ILC metastasis, a liver ILC metastasis, or a skin ILC metastasis. In some embodiments, the ILC metastasis is a female reproductive system ILC metastasis. In some embodiments, the methods provided herein comprise acquiring knowledge that an ILC metastasis comprises an alteration in BRCA1 in a sample from an individual having a bone ILC metastasis. In some embodiments, the methods provided herein comprise acquiring knowledge that an ILC metastasis comprises an alteration in BRCA1 in a sample from an individual having a female reproductive system ILC metastasis. In some embodiments, the methods provided herein comprise acquiring knowledge that an ILC metastasis comprises an alteration in BRCA1 in a sample from an individual having a gastrointestinal ILC metastasis. In some embodiments, the methods provided herein comprise acquiring knowledge that an ILC metastasis comprises an alteration in BRCA1 in a sample from an individual having a liver ILC metastasis. In some embodiments, the methods provided herein comprise acquiring knowledge that an ILC metastasis comprises an alteration in BRCA1 in a sample from an individual having a skin ILC metastasis. In some embodiments, the methods provided herein comprise detecting an alteration in BRCA1 in a sample from an individual having a bone ILC metastasis. In some embodiments, the methods provided herein comprise detecting an alteration in BRCA1 in a sample from an individual having a female reproductive system ILC metastasis. In some embodiments, the methods provided herein comprise detecting an alteration in BRCA1 in a sample from an individual having a gastrointestinal ILC metastasis. In some embodiments, the methods provided herein comprise detecting an alteration in BRCA1 in a sample from an individual having a liver ILC metastasis. In some embodiments, the methods provided herein comprise detecting an alteration in BRCA1 in a sample from an individual having a skin ILC metastasis.

In some embodiments, the methods provided herein comprise acquiring knowledge that an ILC metastasis comprises an alteration in BRCA2 in a sample from an individual having an ILC metastasis. An exemplary nucleic acid sequence of BRCA2 is available as Transcript ID NM_000059, provided herein as SEQ ID NO: 4, and available at the website: www[dot]ncbi[dot]nlm[dot]nih[dot]gov/nuccore/NM_000059.

An exemplary amino acid sequence of a BRCA2 polypeptide is provided herein in SEQ ID NO: 28.

(SEQ ID NO: 28)
MPIGSKERPTFFEIFKTRCNKADLGPISLNWFEELSSEAPPYNSEPAEESEHKNNNYEPNLFKTPQRK
PSYNQLASTPIIFKEQGLTLPLYQSPVKELDKFKLDLGRNVPNSRHKSLRTVKTKMDQADDVSCPLLN
SCLSESPVVLQCTHVTPQRDKSVVCGSLFHTPKFVKGRQTPKHISESLGAEVDPDMSWSSSLATPPTL
SSTVLIVRNEEASETVFPHDTTANVKSYFSNHDESLKKNDRFIASVTDSENTNQREAASHGFGKTSGN
SFKVNSCKDHIGKSMPNVLEDEVYETVVDTSEEDSFSLCFSKCRTKNLQKVRTSKTRKKIFHEANADE
CEKSKNQVKEKYSFVSEVEPNDTDPLDSNVANQKPFESGSDKISKEVVPSLACEWSQLTLSGLNGAQM
EKIPLLHISSCDQNISEKDLLDTENKRKKDFLTSENSLPRISSLPKSEKPLNEETVVNKRDEEQHLES
HTDCILAVKQAISGTSPVASSFQGIKKSIFRIRESPKETFNASFSGHMTDPNFKKETEASESGLEIHT
VCSQKEDSLCPNLIDNGSWPATTTQNSVALKNAGLISTLKKKTNKFIYAIHDETSYKGKKIPKDQKSE
LINCSAQFEANAFEAPLTFANADSGLLHSSVKRSCSQNDSEEPTLSLTSSFGTILRKCSRNETCSNNT
VISQDLDYKEAKCNKEKLQLFITPEADSLSCLQEGQCENDPKSKKVSDIKEEVLAAACHPVQHSKVEY
SDTDFQSQKSLLYDHENASTLILTPTSKDVLSNLVMISRGKESYKMSDKLKGNNYESDVELTKNIPME
KNQDVCALNENYKNVELLPPEKYMRVASPSRKVQFNQNTNLRVIQKNQEETTSISKITVNPDSEELFS
DNENNFVFQVANERNNLALGNTKELHETDLTCVNEPIFKNSTMVLYGDTGDKQATQVSIKKDLVYVLA
EENKNSVKQHIKMTLGQDLKSDISLNIDKIPEKNNDYMNKWAGLLGPISNHSFGGSFRTASNKEIKLS
EHNIKKSKMFFKDIEEQYPTSLACVEIVNTLALDNQKKLSKPQSINTVSAHLQSSVVVSDCKNSHITP
QMLFSKQDENSNHNLTPSQKAEITELSTILEESGSQFEFTQFRKPSYILQKSTFEVPENQMTILKTTS
EECRDADLHVIMNAPSIGQVDSSKQFEGTVEIKRKFAGLLKNDCNKSASGYLTDENEVGFRGFYSAHG
TKLNVSTEALQKAVKLESDIENISEETSAEVHPISLSSSKCHDSVVSMFKIENHNDKTVSEKNNKCQL
ILQNNIEMTTGTFVEEITENYKRNTENEDNKYTAASRNSHNLEFDGSDSSKNDTVCIHKDETDLLFTD
QHNICLKLSGQFMKEGNTQIKEDLSDLTFLEVAKAQEACHGNTSNKEQLTATKTEQNIKDFETSDTFF
QTASGKNISVAKESFNKIVNFFDQKPEELHNFSLNSELHSDIRKNKMDILSYEETDIVKHKILKESVP
VGTGNQLVTFQGQPERDEKIKEPTLLGFHTASGKKVKIAKESLDKVKNLFDEKEQGTSEITSFSHQWA
KTLKYREACKDLELACETIEITAAPKCKEMQNSLNNDKNLVSIETVVPPKLLSDNLCRQTENLKTSKS
IFLKVKVHENVEKETAKSPATCYTNQSPYSVIENSALAFYTSCSRKTSVSQTSLLEAKKWLREGIFDG
QPERINTADYVGNYLYENNSNSTIAENDKNHLSEKQDTYLSNSSMSNSYSYHSDEVYNDSGYLSKNKL
DSGIEPVLKNVEDQKNTSFSKVISNVKDANAYPQTVNEDICVEELVTSSSPCKNKNAAIKLSISNSNN
FEVGPPAFRIASGKIVCVSHETIKKVKDIFTDSFSKVIKENNENKSKICQTKIMAGCYEALDDSEDIL
HNSLDNDECSTHSHKVFADIQSEEILQHNQNMSGLEKVSKISPCDVSLETSDICKCSIGKLHKSVSSA
NTCGIFSTASGKSVQVSDASLQNARQVFSEIEDSTKQVFSKVLFKSNEHSDQLTREENTAIRTPEHLI
SQKGFSYNVVNSSAFSGESTASGKQVSILESSLHKVKGVLEEFDLIRTEHSLHYSPTSRQNVSKILPR
VDKRNPEHCVNSEMEKTCSKEFKLSNNLNVEGGSSENNHSIKVSPYLSQFQQDKQQLVLGTKVSLVEN
IHVLGKEQASPKNVKMEIGKTETFSDVPVKTNIEVCSTYSKDSENYFETEAVEIAKAFMEDDELTDSK
LPSHATHSLFTCPENEEMVLSNSRIGKRRGEPLILVGEPSIKRNLLNEFDRIIENQEKSLKASKSTPD
GTIKDRRLFMHHVSLEPITCVPFRTTKERQEIQNPNFTAPGQEFLSKSHLYEHLTLEKSSSNLAVSGH
PFYQVSATRNEKMRHLITTGRPTKVFVPPFKTKSHFHRVEQCVRNINLEENRQKQNIDGHGSDDSKNK
INDNEIHQFNKNNSNQAVAVTFTKCEEEPLDLITSLQNARDIQDMRIKKKQRQRVFPQPGSLYLAKTS
TLPRISLKAAVGGQVPSACSHKQLYTYGVSKHCIKINSKNAESFQFHTEDYFGKESLWTGKGIQLADG
GWLIPSNDGKAGKEEFYRALCDTPGVDPKLISRIWVYNHYRWIIWKLAAMECAFPKEFANRCLSPERV
LLQLKYRYDTEIDRSRRSAIKKIMERDDTAAKTLVLCVSDIISLSANISETSSNKTSSADTQKVAIIE
LTDGWYAVKAQLDPPLLAVLKNGRLTVGQKIILHGAELVGSPDACTPLEAPESLMLKISANSTRPARW
YTKLGFFPDPRPFPLPLSSLFSDGGNVGCVDVIIQRAYPIQWMEKTSSGLYIFRNEREEEKEAAKYVE
AQQKRLEALFTKIQEEFEEHEENTTKPYLPSRALTRQQVRALQDGAELYEAVKNAADPAYLEGYFSEE
QLRALNNHRQMLNDKKQAQIQLEIRKAMESAEQKEQGLSRDVTTVWKLRIVSYSKKEKDSVILSIWRP
SSDLYSLLTEGKRYRIYHLATSKSKSKSERANIQLAATKKTQYQQLPVSDEILFQIYQPREPLHESKF
LDPDFQPSCSEVDLIGFVVSVVKKTGLAPFVYLSDECYNLLAIKFWIDLNEDIIKPHMLIAASNLQWR
PESKSGLLTLFAGDFSVFSASPKEGHFQETENKMKNTVENIDILCNEAENKLMHILHANDPKWSTPTK
DCTSGPYTAQIIPGTGNKLLMSSPNCEIYYQSPLSLCMAKRKSVSTPVSAQMTSKSCKGEKEIDDQKN
CKKRRALDFLSRLPLPPPVSPICTFVSPAAQKAFQPPRSCGTKYETPIKKKELNSPQMTPFKKENEIS
LLESNSIADEELALINTQALLSGSTGEKQFISVSESTRTAPTSSEDYLRLKRRCTTSLIKEQESSQAS
TEECEKNKQDTITTKKYI

In some embodiments, acquiring knowledge that the ILC metastasis comprises an alteration in BRCA2 comprises detecting the alteration in a sample, e.g., in a sample from an ILC tumor, obtained from an individual. In some embodiments, the methods provided herein comprise detecting an alteration in BRCA2 in a sample from an individual having an ILC metastasis. In some embodiments, the methods further comprise administering an effective amount of an anti-cancer agent responsive to knowledge of the presence of an alteration in BRCA2 in a sample from an individual having an ILC metastasis. In some embodiments, the methods further comprise providing a report to a party. In some embodiments, the ILC metastasis is a bone ILC metastasis, a female reproductive system ILC metastasis, a gastrointestinal ILC metastasis, a liver ILC metastasis, or a skin ILC metastasis. In some embodiments, the ILC metastasis is a female reproductive system ILC metastasis. In some embodiments, the methods provided herein comprise acquiring knowledge that an ILC metastasis comprises an alteration in BRCA2 in a sample from an individual having a bone ILC metastasis. In some embodiments, the methods provided herein comprise acquiring knowledge that an ILC metastasis comprises an alteration in BRCA2 in a sample from an individual having a female reproductive system ILC metastasis. In some embodiments, the methods provided herein comprise acquiring knowledge that an ILC metastasis comprises an alteration in BRCA2 in a sample from an individual having a gastrointestinal ILC metastasis. In some embodiments, the methods provided herein comprise acquiring knowledge that an ILC metastasis comprises an alteration in BRCA2 in a sample from an individual having a liver ILC metastasis. In some embodiments, the methods provided herein comprise acquiring knowledge that an ILC metastasis comprises an alteration in BRCA2 in a sample from an individual having a skin ILC metastasis. In some embodiments, the methods provided herein comprise detecting an alteration in BRCA2 in a sample from an individual having a bone ILC metastasis. In some embodiments, the methods provided herein comprise detecting an alteration in BRCA2 in a sample from an individual having a female reproductive system ILC metastasis. In some embodiments, the methods provided herein comprise detecting an alteration in BRCA2 in a sample from an individual having a gastrointestinal ILC metastasis. In some embodiments, the methods provided herein comprise detecting an alteration in BRCA2 in a sample from an individual having a liver ILC metastasis. In some embodiments, the methods provided herein comprise detecting an alteration in BRCA2 in a sample from an individual having a skin ILC metastasis.

In some embodiments, the methods provided herein comprise acquiring knowledge that an ILC metastasis comprises an alteration in BRCA1 and BRCA2 in a sample from an individual having an ILC metastasis. In some embodiments, acquiring knowledge that the ILC metastasis comprises an alteration in BRCA1 and BRCA2 comprises detecting the alteration in a sample, e.g., in a sample from an ILC tumor, obtained from an individual. In some embodiments, the methods provided herein comprise detecting an alteration in BRCA1 and BRCA2 in a sample from an individual having an ILC metastasis. In some embodiments, the methods further comprise administering an effective amount of an anti-cancer agent responsive to knowledge of the presence of an alteration in BRCA1 and BRCA2 in a sample from an individual having an ILC metastasis. In some embodiments, the methods further comprise providing a report to a party. In some embodiments, the ILC metastasis is a bone ILC metastasis, a female reproductive system ILC metastasis, a gastrointestinal ILC metastasis, a liver ILC metastasis, or a skin ILC metastasis. In some embodiments, the ILC metastasis is a female reproductive system ILC metastasis. In some embodiments, the methods provided herein comprise acquiring knowledge that an ILC metastasis comprises an alteration in BRCA1 and BRCA2 in a sample from an individual having a bone ILC metastasis. In some embodiments, the methods provided herein comprise acquiring knowledge that an ILC metastasis comprises an alteration in BRCA1 and BRCA2 in a sample from an individual having a female reproductive system ILC metastasis. In some embodiments, the methods provided herein comprise acquiring knowledge that an ILC metastasis comprises an alteration in BRCA1 and BRCA2 in a sample from an individual having a gastrointestinal ILC metastasis. In some embodiments, the methods provided herein comprise acquiring knowledge that an ILC metastasis comprises an alteration in BRCA1 and BRCA2 in a sample from an individual having a liver ILC metastasis. In some embodiments, the methods provided herein comprise acquiring knowledge that an ILC metastasis comprises an alteration in BRCA1 and BRCA2 in a sample from an individual having a skin ILC metastasis. In some embodiments, the methods provided herein comprise detecting an alteration in BRCA1 and BRCA2 in a sample from an individual having a bone ILC metastasis. In some embodiments, the methods provided herein comprise detecting an alteration in BRCA1 and BRCA2 in a sample from an individual having a female reproductive system ILC metastasis. In some embodiments, the methods provided herein comprise detecting an alteration in BRCA1 and BRCA2 in a sample from an individual having a gastrointestinal ILC metastasis. In some embodiments, the methods provided herein comprise detecting an alteration in BRCA1 and BRCA2 in a sample from an individual having a liver ILC metastasis. In some embodiments, the methods provided herein comprise detecting an alteration in BRCA1 and BRCA2 in a sample from an individual having a skin ILC metastasis.

In some embodiments, the methods provided herein comprise acquiring knowledge that an ILC metastasis comprises an alteration in ESR1 in a sample from an individual having an ILC metastasis. An exemplary nucleic acid sequence of ESR1 is available as Transcript ID NM_000125, provided herein as SEQ ID NO: 5, and available at the website: www[dot]ncbi[dot]nlm[dot]nih[dot]gov/nuccore/NM_000125.

An exemplary amino acid sequence of an ESR1 polypeptide is provided herein in SEQ ID NO: 29.

(SEQ ID NO: 29)
MTMTLHTKASGMALLHQIQGNELEPLNRPQLKIPLERPLGEVYLDSSKPAVYNYPEGAAYEFNAAAAA
NAQVYGQTGLPYGPGSEAAAFGSNGLGGFPPLNSVSPSPLMLLHPPPQLSPFLQPHGQQVPYYLENEP
SGYTVREAGPPAFYRPNSDNRRQGGRERLASTNDKGSMAMESAKETRYCAVCNDYASGYHYGVWSCEG
CKAFFKRSIQGHNDYMCPATNQCTIDKNRRKSCQACRLRKCYEVGMMKGGIRKDRRGGRMLKHKRQRD
DGEGRGEVGSAGDMRAANLWPSPLMIKRSKKNSLALSLTADQMVSALLDAEPPILYSEYDPTRPFSEA
SMMGLLTNLADRELVHMINWAKRVPGFVDLTLHDQVHLLECAWLEILMIGLVWRSMEHPGKLLFAPNL
LLDRNQGKCVEGMVEIFDMLLATSSRFRMMNLQGEEFVCLKSIILLNSGVYTFLSSTLKSLEEKDHIH
RVLDKITDTLIHLMAKAGLTLQQQHQRLAQLLLILSHIRHMSNKGMEHLYSMKCKNVVPLYDLLLEML
DAHRLHAPTSRGGASVEETDQSHLATAGSTSSHSLQKYYITGEAEGFPATV

In some embodiments, acquiring knowledge that the ILC metastasis comprises an alteration in ESR1 comprises detecting the alteration in a sample, e.g., in a sample from an ILC tumor, obtained from an individual. In some embodiments, the methods provided herein comprise detecting an alteration in ESR1 in a sample from an individual having an ILC metastasis. In some embodiments, the methods further comprise administering an effective amount of an anti-cancer agent responsive to knowledge of the presence of an alteration in ESR1 in a sample from an individual having an ILC metastasis. In some embodiments, the alteration comprises one or more of a substitution of one or more nucleotides, an insertion of one or more nucleotides, or a deletion of one or more nucleotides in ESR1. In some embodiments, the methods further comprise providing a report to a party. In some embodiments, the ILC metastasis is a bone ILC metastasis, a female reproductive system ILC metastasis, a gastrointestinal ILC metastasis, a liver ILC metastasis, or a skin ILC metastasis. In some embodiments, the ILC metastasis is a liver ILC metastasis or a gastrointestinal ILC metastasis. In some embodiments, the methods provided herein comprise acquiring knowledge that an ILC metastasis comprises an alteration in ESR1 in a sample from an individual having a bone ILC metastasis. In some embodiments, the methods provided herein comprise acquiring knowledge that an ILC metastasis comprises an alteration in ESR1 in a sample from an individual having a female reproductive system ILC metastasis. In some embodiments, the methods provided herein comprise acquiring knowledge that an ILC metastasis comprises an alteration in ESR1 in a sample from an individual having a gastrointestinal ILC metastasis. In some embodiments, the methods provided herein comprise acquiring knowledge that an ILC metastasis comprises an alteration in ESR1 in a sample from an individual having a liver ILC metastasis. In some embodiments, the methods provided herein comprise acquiring knowledge that an ILC metastasis comprises an alteration in ESR1 in a sample from an individual having a skin ILC metastasis. In some embodiments, the methods provided herein comprise detecting an alteration in ESR1 in a sample from an individual having a bone ILC metastasis. In some embodiments, the methods provided herein comprise detecting an alteration in ESR1 in a sample from an individual having a female reproductive system ILC metastasis. In some embodiments, the methods provided herein comprise detecting an alteration in ESR1 in a sample from an individual having a gastrointestinal ILC metastasis. In some embodiments, the methods provided herein comprise detecting an alteration in ESR1 in a sample from an individual having a liver ILC metastasis. In some embodiments, the methods provided herein comprise detecting an alteration in ESR1 in a sample from an individual having a skin ILC metastasis. In some embodiments, an alteration in ESR1 is an activating mutation, a gain-of-function mutation, a copy number alteration (e.g., an amplification), a rearrangement, or a gene fusion. In some embodiments, the alteration in ESR1 results in a constitutively active polypeptide or protein encoded by ESR1. In some embodiments, the alteration in ESR1 results in a polypeptide or protein encoded by ESR1 that has increased activity, e.g., as compared to a polypeptide or protein encoded by an ESR1 gene without the alteration. In some embodiments, the alteration in ESR1 results in constitutive expression of ESR1. In some embodiments, the alteration in ESR1 results in overexpression of ESR1. In some embodiments, the alteration in ESR1 is an alteration resulting in one or more amino acid substitutions in a polypeptide encoded by the ESR1 gene. In some embodiments, the one or more amino acid substitutions are at amino acid position E380, V418, S463, V533, L536, Y537, D538, or any combination thereof, wherein the numbering of the residues is according to SEQ ID NO: 29, or according to the amino acid sequence of a human ESR1 protein. In some embodiments, the one or more amino acid substitutions in a polypeptide encoded by the ESR1 gene comprise a E380Q, V418E, S463P, V533M, L536Q, L536H, L536P, L536R, Y537S, Y537N, Y537C, Y537D, or D538G amino acid substitution, or any combination thereof, wherein the numbering of the residues is according to SEQ ID NO: 29, or according to the amino acid sequence of a human ESR1 protein. In some embodiments, the alteration in ESR1 is an alteration resulting in a deletion of one or more amino acids in a polypeptide encoded by the ESR1 gene. In some embodiments, the deletion of one or more amino acids in the polypeptide encoded by the ESR1 gene is a deletion of amino acid V422 (V422del) and/or a deletion of amino acids V533-L536 (V533_L536del) in a polypeptide encoded by the ESR1 gene, wherein the numbering of the residues is according to SEQ ID NO: 29, or according to the amino acid sequence of a human ESR1 protein.

In some embodiments, the methods provided herein comprise acquiring knowledge that an ILC metastasis comprises an alteration in NF1 in a sample from an individual having an ILC metastasis. An exemplary nucleic acid sequence of NF1 is available as Transcript ID NM_001042492, provided herein as SEQ ID NO: 6, and available at the website: www[dot]ncbi[dot]nlm[dot]nih[dot]gov/nuccore/NM_001042492; or as Transcript ID NM_001128147, provided herein as SEQ ID NO: 7, and available at the website: www[dot]ncbi[dot]nlm[dot]nih[dot]gov/nuccore/NM_001128147.

Exemplary amino acid sequences of an NF1 polypeptide are provided herein in SEQ ID NO: and SEQ ID NO: 31.

(SEQ ID NO: 30)
MAAHRPVEWVQAVVSRFDEQLPIKTGQQNTHTKVSTEHNKECLINISKYKFSLVISGLTTILKNVNNM
RIFGEAAEKNLYLSQLIILDTLEKCLAGQPKDTMRLDETMLVKQLLPEICHELHTCREGNQHAAELRN
SASGVLFSLSCNNFNAVFSRISTRLQELTVCSEDNVDVHDIELLQYINVDCAKLKRLLKETAFKFKAL
KKVAQLAVINSLEKAFWNWVENYPDEFTKLYQIPQTDMAECAEKLFDLVDGFAESTKRKAAVWPLQII
LLILCPEIIQDISKDVVDENNMNKKLFLDSLRKALAGHGGSRQLTESAAIACVKLCKASTYINWEDNS
VIFLLVQSMVVDLKNLLENPSKPFSRGSQPADVDLMIDCLVSCFRISPHNNQHFKICLAQNSPSTFHY
VLVNSLHRIITNSALDWWPKIDAVYCHSVELRNMFGETLHKAVQGCGAHPAIRMAPSLTFKEKVTSLK
FKEKPTDLETRSYKYLLLSMVKLIHADPKLLLCNPRKQGPETQGSTAELITGLVQLVPQSHMPEIAQE
AMEALLVLHQLDSIDLWNPDAPVETFWEISSQMLFYICKKLTSHQMLSSTEILKWLREILICRNKFLL
KNKQADRSSCHFLLFYGVGCDIPSSGNTSQMSMDHEELLRTPGASLRKGKGNSSMDSAAGCSGTPPIC
RQAQTKLEVALYMFLWNPDTEAVLVAMSCFRHLCEEADIRCGVDEVSVHNLLPNYNTFMEFASVSNMM
STGRAALQKRVMALLRRIEHPTAGNTEAWEDTHAKWEQATKLILNYPKAKMEDGQAAESLHKTIVKRR
MSHVSGGGSIDLSDTDSLQEWINMTGFLCALGGVCLQQRSNSGLATYSPPMGPVSERKGSMISVMSSE
GNADTPVSKFMDRLLSLMVCNHEKVGLQIRTNVKDLVGLELSPALYPMLENKLKNTISKFFDSQGQVL
LTDTNTQFVEQTIAIMKNLLDNHTEGSSEHLGQASIETMMLNLVRYVRVLGNMVHAIQIKTKLCQLVE
VMMARRDDLSFCQEMKFRNKMVEYLTDWVMGTSNQAADDDVKCLTRDLDQASMEAVVSLLAGLPLQPE
EGDGVELMEAKSQLFLKYFTLFMNLLNDCSEVEDESAQTGGRKRGMSRRLASLRHCTVLAMSNLLNAN
VDSGLMHSIGLGYHKDLQTRATFMEVLTKILQQGTEFDTLAETVLADRFERLVELVTMMGDQGELPIA
MALANVVPCSQWDELARVLVTLEDSRHLLYQLLWNMESKEVELADSMQTLERGNSLASKIMTFCEKVY
GATYLQKLLDPLLRIVITSSDWQHVSFEVDPTRLEPSESLEENQRNLLQMTEKFFHAIISSSSEFPPQ
LRSVCHCLYQATCHSLLNKATVKEKKENKKSVVSQRFPQNSIGAVGSAMFLRFINPAIVSPYEAGILD
KKPPPRIERGLKLMSKILQSIANHVLFTKEEHMRPFNDFVKSNFDAARRFFLDIASDCPTSDAVNHSL
SFISDGNVLALHRLLWNNQEKIGQYLSSNRDHKAVGRRPFDKMATLLAYLGPPEHKPVADTHWSSLNL
TSSKFEEFMTRHQVHEKEEFKALKTLSIFYQAGTSKAGNPIFYYVARRFKTGQINGDLLIYHVLLTLK
PYYAKPYEIVVDLTHTGPSNRFKTDFLSKWFVVFPGFAYDNVSAVYIYNCNSWVREYTKYHERLLTGL
KGSKRLVFIDCPGKLAEHIEHEQQKLPAATLALEEDLKVFHNALKLAHKDTKVSIKVGSTAVQVTSAE
RTKVLGQSVFLNDIYYASEIEEICLVDENQFTLTIANQGTPLTFMHQECEAIVQSIIHIRTRWELSQP
DSIPQHTKIRPKDVPGTLLNIALLNLGSSDPSLRSAAYNLLCALTCTFNLKIEGQLLETSGLCIPANN
TLFIVSISKTLAANEPHLTLEFLEECISGFSKSSIELKHLCLEYMTPWLSNLVRFCKHNDDAKRQRVT
AILDKLITMTINEKQMYPSIQAKIWGSLGQITDLLDVVLDSFIKTSATGGLGSIKAEVMADTAVALAS
GNVKLVSSKVIGRMCKIIDKTCLSPTPTLEQHLMWDDIAILARYMLMLSENNSLDVAAHLPYLFHVVT
FLVATGPLSLRASTHGLVINIIHSLCTCSQLHFSEETKQVLRLSLTEFSLPKFYLLFGISKVKSAAVI
AFRSSYRDRSFSPGSYERETFALTSLETVTEALLEIMEACMRDIPTCKWLDQWTELAQRFAFQYNPSL
QPRALVVFGCISKRVSHGQIKQIIRILSKALESCLKGPDTYNSQVLIEATVIALTKLQPLLNKDSPLH
KALFWVAVAVLQLDEVNLYSAGTALLEQNLHTLDSLRIFNDKSPEEVEMAIRNPLEWHCKQMDHFVGL
NFNSNFNFALVGHLLKGYRHPSPAIVARTVRILHTLLTLVNKHRNCDKFEVNTQSVAYLAALLTVSEE
VRSRCSLKHRKSLLLTDISMENVPMDTYPIHHGDPSYRTLKETQPWSSPKGSEGYLAATYPTVGQTSP
RARKSMSLDMGQPSQANTKKLLGTRKSFDHLISDTKAPKRQEMESGITTPPKMRRVAETDYEMETQRI
SSSQQHPHLRKVSVSESNVLLDEEVLTDPKIQALLLTVLATLVKYTTDEFDQRILYEYLAEASVVFPK
VFPVVHNLLDSKINTLLSLCQDPNLLNPIHGIVQSVVYHEESPPQYQTSYLQSFGENGLWRFAGPFSK
QTQIPDYAELIVKFLDALIDTYLPGIDEETSEESLLTPTSPYPPALQSQLSITANLNLSNSMTSLATS
QHSPGIDKENVELSPTTGHCNSGRTRHGSASQVQKQRSAGSFKRNSIKKIV
(SEQ ID NO: 31)
MAAHRPVEWVQAVVSRFDEQLPIKTGQQNTHTKVSTEHNKECLINISKYKFSLVISGLTTILKNVNNM
RIFGEAAEKNLYLSQLIILDTLEKCLAGQPKDTMRLDETMLVKQLLPEICHELHTCREGNQHAAELRN
SASGVLFSLSCNNFNAVESRISTRLQELTVCSEDNVDVHDIELLQYINVDCAKLKRLLKETAFKFKAL
KKVAQLAVINSLEKAFWNWVENYPDEFTKLYQIPQTDMAECAEKLFDLVDGFAESTKRKAAVWPLQII
LLILCPEIIQDISKDVVDENNMNKKLFLDSLRKALAGHGGSRQLTESAAIACVKLCKASTYINWEDNS
VIFLLVQSMVVDLKNLLENPSKPFSRGSQPADVDLMIDCLVSCFRISPHNNQHFKICLAQNSPSTFHY
VLVNSLHRIITNSALDWWPKIDAVYCHSVELRNMFGETLHKAVQGCGAHPAIRMAPSLTFKEKVTSLK
FKEKPTDLETRSYKYLLLSMVKLIHADPKLLLCNPRKQGPETQGSTAELITGLVQLVPQSHMPEIAQE
AMEALLVLHQLDSIDLWNPDAPVETFWEIRYMYFYFLNSTFKFYFVELS

In some embodiments, acquiring knowledge that the ILC metastasis comprises an alteration in NF1 comprises detecting the alteration in a sample, e.g., in a sample from an ILC tumor, obtained from an individual. In some embodiments, the methods provided herein comprise detecting an alteration in NF1 in a sample from an individual having an ILC metastasis. In some embodiments, the methods further comprise administering an effective amount of an anti-cancer agent responsive to knowledge of the presence of an alteration in NF1 in a sample from an individual having an ILC metastasis. In some embodiments, the alteration comprises one or more of a substitution of one or more nucleotides, an insertion of one or more nucleotides, or a deletion of one or more nucleotides in NF1. In some embodiments, the methods further comprise providing a report to a party. In some embodiments, the ILC metastasis is a bone ILC metastasis, a female reproductive system ILC metastasis, a gastrointestinal ILC metastasis, a liver ILC metastasis, or a skin ILC metastasis. In some embodiments, the methods provided herein comprise acquiring knowledge that an ILC metastasis comprises an alteration in NF1 in a sample from an individual having a bone ILC metastasis. In some embodiments, the methods provided herein comprise acquiring knowledge that an ILC metastasis comprises an alteration in NF1 in a sample from an individual having a female reproductive system ILC metastasis. In some embodiments, the methods provided herein comprise acquiring knowledge that an ILC metastasis comprises an alteration in NF1 in a sample from an individual having a gastrointestinal ILC metastasis. In some embodiments, the methods provided herein comprise acquiring knowledge that an ILC metastasis comprises an alteration in NF1 in a sample from an individual having a liver ILC metastasis. In some embodiments, the methods provided herein comprise acquiring knowledge that an ILC metastasis comprises an alteration in NF1 in a sample from an individual having a skin ILC metastasis. In some embodiments, the methods provided herein comprise detecting an alteration in NF1 in a sample from an individual having a bone ILC metastasis. In some embodiments, the methods provided herein comprise detecting an alteration in NF1 in a sample from an individual having a female reproductive system ILC metastasis. In some embodiments, the methods provided herein comprise detecting an alteration in NF1 in a sample from an individual having a gastrointestinal ILC metastasis. In some embodiments, the methods provided herein comprise detecting an alteration in NF1 in a sample from an individual having a liver ILC metastasis. In some embodiments, the methods provided herein comprise detecting an alteration in NF1 in a sample from an individual having a skin ILC metastasis. In some embodiments, the alteration in NF1 is an inactivating mutation, a loss-of-function mutation, a copy number alteration (e.g., a deletion), a rearrangement, or a gene fusion. In some embodiments, the alteration in NF1 results in an inactive polypeptide or protein encoded by NF1. In some embodiments, the alteration in NF1 results in a polypeptide or protein encoded by NF1 that has reduced activity, e.g., as compared to a polypeptide or protein encoded by an NF1 gene without the alteration. In some embodiments, the alteration in NF1 results in reduced expression of NF1. In some embodiments, the alteration in NF1 results in loss of expression of NF1.

In some embodiments, the methods provided herein comprise acquiring knowledge that an ILC metastasis comprises an alteration in RB1 in a sample from an individual having an ILC metastasis. An exemplary nucleic acid sequence of RB1 is available as Transcript ID NM_000321, provided herein as SEQ ID NO: 8, and available at the website: www[dot]ncbi[dot]nlm[dot]nih[dot]gov/nuccore/NM_000321.

An exemplary amino acid sequence of an RB1 polypeptide is provided herein in SEQ ID NO: 32.

(SEQ ID NO: 32)
MPPKTPRKTAATAAAAAAEPPAPPPPPPPEEDPEQDSGPEDLPLVRLEFEETEEPDFTALCQKLKIPD
HVRERAWLTWEKVSSVDGVLGGYIQKKKELWGICIFIAAVDLDEMSFTFTELQKNIEISVHKFFNLLK
EIDTSTKVDNAMSRLLKKYDVLFALFSKLERTCELIYLTQPSSSISTEINSALVLKVSWITFLLAKGE
VLQMEDDLVISFQLMLCVLDYFIKLSPPMLLKEPYKTAVIPINGSPRTPRRGQNRSARIAKQLENDTR
IIEVLCKEHECNIDEVKNVYFKNFIPFMNSLGLVTSNGLPEVENLSKRYEEIYLKNKDLDARLFLDHD
KTLQTDSIDSFETQRTPRKSNLDEEVNVIPPHTPVRTVMNTIQQLMMILNSASDQPSENLISYENNCT
VNPKESILKRVKDIGYIFKEKFAKAVGQGCVEIGSQRYKLGVRLYYRVMESMLKSEEERLSIQNFSKL
LNDNIFHMSLLACALEVVMATYSRSTSQNLDSGTDLSFPWILNVLNLKAFDFYKVIESFIKAEGNLTR
EMIKHLERCEHRIMESLAWLSDSPLFDLIKQSKDREGPTDHLESACPLNLPLQNNHTAADMYLSPVRS
PKKKGSTTRVNSTANAETQATSAFQTQKPLKSTSLSLFYKKVYRLAYLRLNTLCERLLSEHPELEHII
WTLFQHTLQNEYELMRDRHLDQIMMCSMYGICKVKNIDLKFKIIVTAYKDLPHAVQETFKRVLIKEEE
YDSIIVFYNSVFMQRLKTNILQYASTRPPTLSPIPHIPRSPYKFPSSPLRIPGGNIYISPLKSPYKIS
EGLPTPTKMTPRSRILVSIGESFGTSEKFQKINQMVCNSDRVLKRSAEGSNPPKPLKKLRFDIEGSDE
ADGSKHLPGESKFQQKLAEMTSTRTRMQKQKMNDSMDTSNKEEK

In some embodiments, acquiring knowledge that the ILC metastasis comprises an alteration in RB1 comprises detecting the alteration in a sample, e.g., in a sample from an ILC tumor, obtained from an individual. In some embodiments, the methods provided herein comprise detecting an alteration in RB1 in a sample from an individual having an ILC metastasis. In some embodiments, the methods further comprise administering an effective amount of an anti-cancer agent responsive to knowledge of the presence of an alteration in RB1 in a sample from an individual having an ILC metastasis. In some embodiments, the methods further comprise providing a report to a party. In some embodiments, the alteration comprises one or more of a substitution of one or more nucleotides, an insertion of one or more nucleotides, or a deletion of one or more nucleotides in RB1. In some embodiments, the ILC metastasis is a bone ILC metastasis, a female reproductive system ILC metastasis, a gastrointestinal ILC metastasis, a liver ILC metastasis, or a skin ILC metastasis. In some embodiments, the methods provided herein comprise acquiring knowledge that an ILC metastasis comprises an alteration in RB1 in a sample from an individual having a bone ILC metastasis. In some embodiments, the methods provided herein comprise acquiring knowledge that an ILC metastasis comprises an alteration in RB1 in a sample from an individual having a female reproductive system ILC metastasis. In some embodiments, the methods provided herein comprise acquiring knowledge that an ILC metastasis comprises an alteration in RB1 in a sample from an individual having a gastrointestinal ILC metastasis. In some embodiments, the methods provided herein comprise acquiring knowledge that an ILC metastasis comprises an alteration in RB1 in a sample from an individual having a liver ILC metastasis. In some embodiments, the methods provided herein comprise acquiring knowledge that an ILC metastasis comprises an alteration in RB1 in a sample from an individual having a skin ILC metastasis. In some embodiments, the methods provided herein comprise detecting an alteration in RB1 in a sample from an individual having a bone ILC metastasis. In some embodiments, the methods provided herein comprise detecting an alteration in RB1 in a sample from an individual having a female reproductive system ILC metastasis. In some embodiments, the methods provided herein comprise detecting an alteration in RB1 in a sample from an individual having a gastrointestinal ILC metastasis. In some embodiments, the methods provided herein comprise detecting an alteration in RB1 in a sample from an individual having a liver ILC metastasis. In some embodiments, the methods provided herein comprise detecting an alteration in RB1 in a sample from an individual having a skin ILC metastasis. In some embodiments, the alteration in RB1 is an inactivating mutation, a loss-of-function mutation, a copy number alteration (e.g., a deletion), a rearrangement, or a gene fusion. In some embodiments, the alteration in RB1 results in an inactive polypeptide or protein encoded by RB1. In some embodiments, the alteration in RB1 results in a polypeptide or protein encoded by RB1 that has reduced activity, e.g., as compared to a polypeptide or protein encoded by an RB1 gene without the alteration. In some embodiments, the alteration in RB1 results in reduced expression of RB1. In some embodiments, the alteration in RB1 results in loss of expression of RB1.

In some embodiments, the methods provided herein comprise acquiring knowledge that an ILC metastasis comprises an alteration in KRAS in a sample from an individual having an ILC metastasis. An exemplary nucleic acid sequence of KRAS is available as Transcript ID NM_004985, provided herein as SEQ ID NO: 9, and available at the website: www[dot]ncbi[dot]nlm[dot]nih[dot]gov/nuccore/NM_004985.

An exemplary amino acid sequence of a KRAS polypeptide is provided herein in SEQ ID NO: 33.

(SEQ ID NO: 33)
MTEYKLVVVGAGGVGKSALTIQLIQNHFVDEYDPTIEDSYRKQVVIDGETCLLDILDTAGQEEYSAMR
DQYMRTGEGFLCVFAINNTKSFEDIHHYREQIKRVKDSEDVPMVLVGNKCDLPSRTVDTKQAQDLARS
YGIPFIETSAKTRQGVDDAFYTLVREIRKHKEKMSKDGKKKKKKSKTKCVIM

In some embodiments, acquiring knowledge that the ILC metastasis comprises an alteration in KRAS comprises detecting the alteration in a sample, e.g., in a sample from an ILC tumor, obtained from an individual. In some embodiments, the methods provided herein comprise detecting an alteration in KRAS in a sample from an individual having an ILC metastasis. In some embodiments, the methods further comprise administering an effective amount of an anti-cancer agent responsive to knowledge of the presence of an alteration in KRAS in a sample from an individual having an ILC metastasis. In some embodiments, the methods further comprise providing a report to a party. In some embodiments, the ILC metastasis is a bone ILC metastasis, a female reproductive system ILC metastasis, a gastrointestinal ILC metastasis, a liver ILC metastasis, or a skin ILC metastasis. In some embodiments, the methods provided herein comprise acquiring knowledge that an ILC metastasis comprises an alteration in KRAS in a sample from an individual having a bone ILC metastasis. In some embodiments, the methods provided herein comprise acquiring knowledge that an ILC metastasis comprises an alteration in KRAS in a sample from an individual having a female reproductive system ILC metastasis. In some embodiments, the methods provided herein comprise acquiring knowledge that an ILC metastasis comprises an alteration in KRAS in a sample from an individual having a gastrointestinal ILC metastasis. In some embodiments, the methods provided herein comprise acquiring knowledge that an ILC metastasis comprises an alteration in KRAS in a sample from an individual having a liver ILC metastasis. In some embodiments, the methods provided herein comprise acquiring knowledge that an ILC metastasis comprises an alteration in KRAS in a sample from an individual having a skin ILC metastasis. In some embodiments, the methods provided herein comprise detecting an alteration in KRAS in a sample from an individual having a bone ILC metastasis. In some embodiments, the methods provided herein comprise detecting an alteration in KRAS in a sample from an individual having a female reproductive system ILC metastasis. In some embodiments, the methods provided herein comprise detecting an alteration in KRAS in a sample from an individual having a gastrointestinal ILC metastasis. In some embodiments, the methods provided herein comprise detecting an alteration in KRAS in a sample from an individual having a liver ILC metastasis. In some embodiments, the methods provided herein comprise detecting an alteration in KRAS in a sample from an individual having a skin ILC metastasis. In some embodiments, the alteration in KRAS is an activating mutation, a gain-of-function mutation, a copy number alteration (e.g., an amplification), a rearrangement, or a gene fusion. In some embodiments, the alteration in KRAS results in a constitutively active polypeptide or protein encoded by KRAS. In some embodiments, the alteration in KRAS results in a polypeptide or protein encoded by KRAS that has increased activity, e.g., as compared to a polypeptide or protein encoded by a KRAS gene without the alteration. In some embodiments, the alteration in KRAS results in constitutive expression of KRAS. In some embodiments, the alteration in KRAS results in overexpression of KRAS. In some embodiments, the alteration in KRAS results in one or more amino acid substitutions in a polypeptide encoded by the KRAS gene. In some embodiments, the one or more amino acid substitutions are at amino acid position G12, G13, L19, Q61, A146, K147, F156, or any combination thereof, wherein the numbering of the residues is according to SEQ ID NO: 33, or according to the amino acid sequence of a human KRAS protein. In some embodiments, the one or more amino acid substitutions in a polypeptide encoded by the KRAS gene comprise a G12V, G12D, G12A, G12R, G12S, G12C, G12L, G13D, L19F, Q61H, Q61K, Q61E, A146T, K147N, or F156L amino acid substitution, or any combination thereof, wherein the numbering of the residues is according to SEQ ID NO: 33, or according to the amino acid sequence of a human KRAS protein.

In some embodiments, the methods provided herein comprise acquiring knowledge that an ILC metastasis comprises an alteration in ERBB2 in a sample from an individual having an ILC metastasis. An exemplary nucleic acid sequence of ERBB2 is available as Transcript ID NM_004448, provided herein as SEQ ID NO: 10, and available at the website: www[dot]ncbi[dot]nlm[dot]nih[dot]gov/nuccore/NM_004448.

An exemplary amino acid sequence of an ERBB2 polypeptide is provided herein in SEQ ID NO: 34.

(SEQ ID NO: 34)
MELAALCRWGLLLALLPPGAASTQVCTGTDMKLRLPASPETHLDMLRHLY
QGCQVVQGNLELTYLPTNASLSFLQDIQEVQGYVLIAHNQVRQVPLQRLR
IVRGTQLFEDNYALAVLDNGDPLNNTTPVTGASPGGLRELQLRSLTEILK
GGVLIQRNPQLCYQDTILWKDIFHKNNQLALTLIDTNRSRACHPCSPMCK
GSRCWGESSEDCQSLTRTVCAGGCARCKGPLPTDCCHEQCAAGCTGPKHS
DCLACLHFNHSGICELHCPALVTYNTDTFESMPNPEGRYTFGASCVTACP
YNYLSTDVGSCTLVCPLHNQEVTAEDGTQRCEKCSKPCARVCYGLGMEHL
REVRAVTSANIQEFAGCKKIFGSLAFLPESFDGDPASNTAPLQPEQLQVF
ETLEEITGYLYISAWPDSLPDLSVFQNLQVIRGRILHNGAYSLTLQGLGI
SWLGLRSLRELGSGLALIHHNTHLCFVHTVPWDQLFRNPHQALLHTANRP
EDECVGEGLACHQLCARGHCWGPGPTQCVNCSQFLRGQECVEECRVLQGL
PREYVNARHCLPCHPECQPQNGSVTCFGPEADQCVACAHYKDPPFCVARC
PSGVKPDLSYMPIWKFPDEEGACQPCPINCTHSCVDLDDKGCPAEQRASP
LTSIISAVVGILLVVVLGVVFGILIKRRQQKIRKYTMRRLLQETELVEPL
TPSGAMPNQAQMRILKETELRKVKVLGSGAFGTVYKGIWIPDGENVKIPV
AIKVLRENTSPKANKEILDEAYVMAGVGSPYVSRLLGICLTSTVQLVTQL
MPYGCLLDHVRENRGRLGSQDLLNWCMQIAKGMSYLEDVRLVHRDLAARN
VLVKSPNHVKITDFGLARLLDIDETEYHADGGKVPIKWMALESILRRRFT
HQSDVWSYGVTVWELMTFGAKPYDGIPAREIPDLLEKGERLPQPPICTID
VYMIMVKCWMIDSECRPRFRELVSEFSRMARDPQRFVVIQNEDLGPASPL
DSTFYRSLLEDDDMGDLVDAEEYLVPQQGFFCPDPAPGAGGMVHHRHRSS
STRSGGGDLTLGLEPSEEEAPRSPLAPSEGAGSDVFDGDLGMGAAKGLQS
LPTHDPSPLQRYSEDPTVPLPSETDGYVAPLTCSPQPEYVNQPDVRPQPP
SPREGPLPAARPAGATLERPKTLSPGKNGVVKDVFAFGGAVENPEYLTPQ
GGAAPQPHPPPAFSPAFDNLYYWDQDPPERGAPPSTFKGTPTAENPEYLG
LDVPV

In some embodiments, acquiring knowledge that the ILC metastasis comprises an alteration in ERBB2 comprises detecting the alteration in a sample, e.g., in a sample from an ILC tumor, obtained from an individual. In some embodiments, the methods provided herein comprise detecting an alteration in ERBB2 in a sample from an individual having an ILC metastasis. In some embodiments, the methods further comprise administering an effective amount of an anti-cancer agent responsive to knowledge of the presence of an alteration in ERBB2 in a sample from an individual having an ILC metastasis. In some embodiments, the methods further comprise providing a report to a party. In some embodiments, the alteration comprises one or more of a substitution of one or more nucleotides, an insertion of one or more nucleotides, or a deletion of one or more nucleotides in ERBB2. In some embodiments, the ILC metastasis is a bone ILC metastasis, a female reproductive system ILC metastasis, a gastrointestinal ILC metastasis, a liver ILC metastasis, or a skin ILC metastasis. In some embodiments, the ILC metastasis is a liver ILC metastasis. In some embodiments, the methods provided herein comprise acquiring knowledge that an ILC metastasis comprises an alteration in ERBB2 in a sample from an individual having a bone ILC metastasis. In some embodiments, the methods provided herein comprise acquiring knowledge that an ILC metastasis comprises an alteration in ERBB2 in a sample from an individual having a female reproductive system ILC metastasis. In some embodiments, the methods provided herein comprise acquiring knowledge that an ILC metastasis comprises an alteration in ERBB2 in a sample from an individual having a gastrointestinal ILC metastasis. In some embodiments, the methods provided herein comprise acquiring knowledge that an ILC metastasis comprises an alteration in ERBB2 in a sample from an individual having a liver ILC metastasis. In some embodiments, the methods provided herein comprise acquiring knowledge that an ILC metastasis comprises an alteration in ERBB2 in a sample from an individual having a skin ILC metastasis. In some embodiments, the methods provided herein comprise detecting an alteration in ERBB2 in a sample from an individual having a bone ILC metastasis. In some embodiments, the methods provided herein comprise detecting an alteration in ERBB2 in a sample from an individual having a female reproductive system ILC metastasis. In some embodiments, the methods provided herein comprise detecting an alteration in ERBB2 in a sample from an individual having a gastrointestinal ILC metastasis. In some embodiments, the methods provided herein comprise detecting an alteration in ERBB2 in a sample from an individual having a liver ILC metastasis. In some embodiments, the methods provided herein comprise detecting an alteration in ERBB2 in a sample from an individual having a skin ILC metastasis. In some embodiments, the alteration in ERBB2 is an activating mutation, a gain-of-function mutation, a copy number alteration (e.g., an amplification), a rearrangement, or a gene fusion. In some embodiments, the alteration in ERBB2 results in a constitutively active polypeptide or protein encoded by ERBB2. In some embodiments, the alteration in ERBB2 results in a polypeptide or protein encoded by ERBB2 that has increased activity, e.g., as compared to a polypeptide or protein encoded by an ERBB2 gene without the alteration. In some embodiments, the alteration in ERBB2 results in constitutive expression of ERBB2. In some embodiments, the alteration in ERBB2 results in overexpression of ERBB2. In some embodiments, the alteration in ERBB2 results in one or more amino acid substitutions in a polypeptide encoded by the ERBB2 gene. In some embodiments, the one or more amino acid substitutions are at amino acid position S310, S653, V659, R678, V697, E717, T733, L755, I767, D769, G776, V777, T798, V842, L869, or any combination thereof, wherein the numbering of the residues is according to SEQ ID NO: 34, or according to the amino acid sequence of a human ERBB2 protein. In some embodiments, the one or more amino acid substitutions in a polypeptide encoded by the ERBB2 gene comprise a S310F, S310Y, S653C, V659D, R678Q, V697L, E717K, T733I, L755S, L755P, I767M, D769N, D769Y, D769H, G776V, V777L, T798I, V842I, or L869R amino acid substitution, or any combination thereof, wherein the numbering of the residues is according to SEQ ID NO: 34, or according to the amino acid sequence of a human ERBB2 protein. In some embodiments, the alteration in ERBB2 results in a deletion of one or more amino acid residues in a polypeptide encoded by the ERBB2 gene. In some embodiments, the deletion of one or more amino acid residues in a polypeptide encoded by the ERBB2 gene is a deletion of amino acids L755-T759 (L755_T759del) and/or amino acids L755-E757 (L755_E757del) in a polypeptide encoded by the ERBB2 gene, wherein the numbering of the residues is according to SEQ ID NO: 34, or according to the amino acid sequence of a human ERBB2 protein. In some embodiments, the alteration in ERBB2 results in an insertion of one or more amino acid residues in a polypeptide encoded by the ERBB2 gene. In some embodiments, the insertion of one or more amino acid residues in a polypeptide encoded by the ERBB2 gene is an insertion of the amino acid sequence YVMA (SEQ ID NO: 51) between amino acid residues A775 and G776 (A775_G776insYVMA), and/or of the amino acid sequence GSP (SEQ ID NO: 52) between amino acid residues P780 and Y781 (P780_Y781insGSP) in a polypeptide encoded by the ERBB2 gene, wherein the numbering of the residues is according to SEQ ID NO: 34, or according to the amino acid sequence of a human ERBB2 protein.

In some embodiments, the methods provided herein comprise acquiring knowledge that an ILC metastasis comprises an alteration in BRAF in a sample from an individual having an ILC metastasis. An exemplary nucleic acid sequence of BRAF is available as Transcript ID NM_004333, provided herein as SEQ ID NO: 11, and available at the website: www[dot]ncbi[dot]nlm[dot]nih[dot]gov/nuccore/NM_004333.

An exemplary amino acid sequence of a BRAF polypeptide is provided herein in SEQ ID NO: 35.

(SEQ ID NO: 35)
MAALSGGGGGGAEPGQALFNGDMEPEAGAGAGAAASSAADPAIPEEVWNI
KQMIKLTQEHIEALLDKFGGEHNPPSIYLEAYEEYTSKLDALQQREQQLL
ESLGNGTDFSVSSSASMDTVTSSSSSSLSVLPSSLSVFQNPTDVARSNPK
SPQKPIVRVFLPNKQRTVVPARCGVTVRDSLKKALMMRGLIPECCAVYRI
QDGEKKPIGWDTDISWLTGEELHVEVLENVPLTTHNFVRKTFFTLAFCDF
CRKLLFQGFRCQTCGYKFHQRCSTEVPLMCVNYDQLDLLFVSKFFEHHPI
PQEEASLAETALTSGSSPSAPASDSIGPQILTSPSPSKSIPIPQPFRPAD
EDHRNQFGQRDRSSSAPNVHINTIEPVNIDDLIRDQGFRGDGGSTTGLSA
TPPASLPGSLTNVKALQKSPGPQRERKSSSSSEDRNRMKTLGRRDSSDDW
EIPDGQITVGQRIGSGSFGTVYKGKWHGDVAVKMLNVTAPTPQQLQAFKN
EVGVLRKTRHVNILLFMGYSTKPQLAIVTQWCEGSSLYHHLHIIETKFEM
IKLIDIARQTAQGMDYLHAKSIIHRDLKSNNIFLHEDLTVKIGDFGLATV
KSRWSGSHQFEQLSGSILWMAPEVIRMQDKNPYSFQSDVYAFGIVLYELM
TGQLPYSNINNRDQIIFMVGRGYLSPDLSKVRSNCPKAMKRLMAECLKKK
RDERPLFPQILASIELLARSLPKIHRSASEPSLNRAGFQTEDESLYACAS
PKTPIQAGGYGAFPVH

In some embodiments, acquiring knowledge that the ILC metastasis comprises an alteration in BRAF comprises detecting the alteration in a sample, e.g., in a sample from an ILC tumor, obtained from an individual. In some embodiments, the methods provided herein comprise detecting an alteration in BRAF in a sample from an individual having an ILC metastasis. In some embodiments, the methods further comprise administering an effective amount of an anti-cancer agent responsive to knowledge of the presence of an alteration in BRAF in a sample from an individual having an ILC metastasis. In some embodiments, the methods further comprise providing a report to a party. In some embodiments, the ILC metastasis is a bone ILC metastasis, a female reproductive system ILC metastasis, a gastrointestinal ILC metastasis, a liver ILC metastasis, or a skin ILC metastasis. In some embodiments, the ILC metastasis is a bone ILC metastasis. In some embodiments, the methods provided herein comprise acquiring knowledge that an ILC metastasis comprises an alteration in BRAF in a sample from an individual having a bone ILC metastasis. In some embodiments, the methods provided herein comprise acquiring knowledge that an ILC metastasis comprises an alteration in BRAF in a sample from an individual having a female reproductive system ILC metastasis. In some embodiments, the methods provided herein comprise acquiring knowledge that an ILC metastasis comprises an alteration in BRAF in a sample from an individual having a gastrointestinal ILC metastasis. In some embodiments, the methods provided herein comprise acquiring knowledge that an ILC metastasis comprises an alteration in BRAF in a sample from an individual having a liver ILC metastasis. In some embodiments, the methods provided herein comprise acquiring knowledge that an ILC metastasis comprises an alteration in BRAF in a sample from an individual having a skin ILC metastasis. In some embodiments, the methods provided herein comprise detecting an alteration in BRAF in a sample from an individual having a bone ILC metastasis. In some embodiments, the methods provided herein comprise detecting an alteration in BRAF in a sample from an individual having a female reproductive system ILC metastasis. In some embodiments, the methods provided herein comprise detecting an alteration in BRAF in a sample from an individual having a gastrointestinal ILC metastasis. In some embodiments, the methods provided herein comprise detecting an alteration in BRAF in a sample from an individual having a liver ILC metastasis. In some embodiments, the methods provided herein comprise detecting an alteration in BRAF in a sample from an individual having a skin ILC metastasis. In some embodiments, the alteration in BRAF is an activating mutation, a gain-of-function mutation, a copy number alteration (e.g., an amplification), a rearrangement, or a gene fusion. In some embodiments, the alteration in BRAF results in a constitutively active polypeptide or protein encoded by BRAF. In some embodiments, the alteration in BRAF results in a polypeptide or protein encoded by BRAF that has increased activity, e.g., as compared to a polypeptide or protein encoded by a BRAF gene without the alteration. In some embodiments, the alteration in BRAF results in constitutive expression of BRAF. In some embodiments, the alteration in BRAF results in overexpression of BRAF. In some embodiments, the alteration in BRAF results in one or more amino acid substitutions in a polypeptide encoded by the BRAF gene. In some embodiments, the one or more amino acid substitutions are at amino acid position D380, G464, G466, S467, G469, L485, L584, E586, D594, V600, or any combination thereof, wherein the numbering of the residues is according to SEQ ID NO: 35 or according to the amino acid sequence of a human BRAF protein. In some embodiments, the one or more amino acid substitutions in a polypeptide encoded by the BRAF gene comprise a D380H, G464R, G466E, S467L, G469A, G469E, G469R, L485F, L584F, E586K, D594N, D594G, or V600E amino acid substitution, or any combination thereof, wherein the numbering of the residues is according to SEQ ID NO: 35, or according to the amino acid sequence of a human BRAF protein.

In some embodiments, the methods provided herein comprise acquiring knowledge that an ILC metastasis comprises an alteration in ARID1A in a sample from an individual having an ILC metastasis. An exemplary nucleic acid sequence of ARID1A is available as Transcript ID NM_006015, provided herein as SEQ ID NO: 12, and available at the website: www[dot]ncbi[dot]nlm[dot]nih[dot]gov/nuccore/NM_006015.

An exemplary amino acid sequence of an ARID1A polypeptide is provided herein in SEQ ID NO: 36.

(SEQ ID NO: 36)
MAAQVAPAAASSIGNPPPPPPSELKKAEQQQREEAGGEAAAAAAAERGEM
KAAAGQESEGPAVGPPQPLGKELQDGAESNGGGGGGGAGSGGGPGAEPDL
KNSNGNAGPRPALNNNLTEPPGGGGGGSSDGVGAPPHSAAAALPPPAYGF
GQPYGRSPSAVAAAAAAVFHQQHGGQQSPGLAALQSGGGGGLEPYAGPQQ
NSHDHGFPNHQYNSYYPNRSAYPPPAPAYALSSPRGGTPGSGAAAAAGSK
PPPSSSASASSSSSSFAQQRFGAMGGGGPSAAGGGTPQPTATPTLNQLLT
SPSSARGYQGYPGGDYSGGPQDGGAGKGPADMASQCWGAAAAAAAAAAAS
GGAQQRSHHAPMSPGSSGGGGQPLARTPQPSSPMDQMGKMRPQPYGGTNP
YSQQQGPPSGPQQGHGYPGQPYGSQTPQRYPMTMQGRAQSAMGGLSYTQQ
IPPYGQQGPSGYGQQGQTPYYNQQSPHPQQQQPPYSQQPPSQTPHAQPSY
QQQPQSQPPQLQSSQPPYSQQPSQPPHQQSPAPYPSQQSTTQQHPQSQPP
YSQPQAQSPYQQQQPQQPAPSTLSQQAAYPQPQSQQSQQTAYSQQRFPPP
QELSQDSFGSQASSAPSMTSSKGGQEDMNLSLQSRPSSLPDLSGSIDDLP
MGTEGALSPGVSTSGISSSQGEQSNPAQSPFSPHTSPHLPGIRGPSPSPV
GSPASVAQSRSGPLSPAAVPGNQMPPRPPSGQSDSIMHPSMNQSSIAQDR
GYMQRNPQMPQYSSPQPGSALSPRQPSGGQIHTGMGSYQQNSMGSYGPQG
GQYGPQGGYPRQPNYNALPNANYPSAGMAGGINPMGAGGQMHGQPGIPPY
GTLPPGRMSHASMGNRPYGPNMANMPPQVGSGMCPPPGGMNRKTQETAVA
MHVAANSIQNRPPGYPNMNQGGMMGTGPPYGQGINSMAGMINPQGPPYSM
GGTMANNSAGMAASPEMMGLGDVKLTPATKMNNKADGTPKTESKSKKSSS
STTTNEKITKLYELGGEPERKMWVDRYLAFTEEKAMGMTNLPAVGRKPLD
LYRLYVSVKEIGGLTQVNKNKKWRELATNLNVGTSSSAASSLKKQYIQCL
YAFECKIERGEDPPPDIFAAADSKKSQPKIQPPSPAGSGSMQGPQTPQST
SSSMAEGGDLKPPTPASTPHSQIPPLPGMSRSNSVGIQDAFNDGSDSTFQ
KRNSMTPNPGYQPSMNTSDMMGRMSYEPNKDPYGSMRKAPGSDPFMSSGQ
GPNGGMGDPYSRAAGPGLGNVAMGPRQHYPYGGPYDRVRTEPGIGPEGNM
STGAPQPNLMPSNPDSGMYSPSRYPPQQQQQQQQRHDSYGNQFSTQGTPS
GSPFPSQQTTMYQQQQQNYKRPMDGTYGPPAKRHEGEMYSVPYSTGQGQP
QQQQLPPAQPQPASQQQAAQPSPQQDVYNQYGNAYPATATAATERRPAGG
PQNQFPFQFGRDRVSAPPGTNAQQNMPPQMMGGPIQASAEVAQQGTMWQG
RNDMTYNYANRQSTGSAPQGPAYHGVNRTDEMLHTDQRANHEGSWPSHGT
RQPPYGPSAPVPPMTRPPPSNYQPPPSMQNHIPQVSSPAPLPRPMENRTS
PSKSPFLHSGMKMQKAGPPVPASHIAPAPVQPPMIRRDITFPPGSVEATQ
PVLKQRRRLTMKDIGTPEAWRVMMSLKSGLLAESTWALDTINILLYDDNS
IMTFNLSQLPGLLELLVEYFRRCLIEIFGILKEYEVGDPGQRTLLDPGRF
SKVSSPAPMEGGEEEEELLGPKLEEEEEEEVVENDEEIAFSGKDKPASEN
SEEKLISKFDKLPVKIVQKNDPFVVDCSDKLGRVQEFDSGLLHWRIGGGD
TTEHIQTHFESKTELLPSRPHAPCPPAPRKHVTTAEGTPGTTDQEGPPPD
GPPEKRITATMDDMLSTRSSTLTEDGAKSSEAIKESSKFPFGISPAQSHR
NIKILEDEPHSKDETPLCTLLDWQDSLAKRCVCVSNTIRSLSFVPGNDFE
MSKHPGLLLILGKLILLHHKHPERKQAPLTYEKEEEQDQGVSCNKVEWWW
DCLEMLRENTLVTLANISGQLDLSPYPESICLPVLDGLLHWAVCPSAEAQ
DPFSTLGPNAVLSPQRLVLETLSKLSIQDNNVDLILATPPFSRLEKLYST
MVRFLSDRKNPVCREMAVVLLANLAQGDSLAARAIAVQKGSIGNLLGFLE
DSLAATQFQQSQASLLHMQNPPFEPTSVDMMRRAARALLALAKVDENHSE
FTLYESRLLDISVSPLMNSLVSQVICDVLFLIGQS

In some embodiments, acquiring knowledge that the ILC metastasis comprises an alteration in ARID1A comprises detecting the alteration in a sample, e.g., in a sample from an ILC tumor, obtained from an individual. In some embodiments, the methods provided herein comprise detecting an alteration in ARID1A in a sample from an individual having an ILC metastasis. In some embodiments, the methods further comprise administering an effective amount of an anti-cancer agent responsive to knowledge of the presence of an alteration in ARID1A in a sample from an individual having an ILC metastasis. In some embodiments, the methods further comprise providing a report to a party. In some embodiments, the alteration comprises one or more of a substitution of one or more nucleotides, an insertion of one or more nucleotides, or a deletion of one or more nucleotides in ARID1A. In some embodiments, the ILC metastasis is a bone ILC metastasis, a female reproductive system ILC metastasis, a gastrointestinal ILC metastasis, a liver ILC metastasis, or a skin ILC metastasis. In some embodiments, the methods provided herein comprise acquiring knowledge that an ILC metastasis comprises an alteration in ARID1A in a sample from an individual having a bone ILC metastasis. In some embodiments, the methods provided herein comprise acquiring knowledge that an ILC metastasis comprises an alteration in ARID1A in a sample from an individual having a female reproductive system ILC metastasis. In some embodiments, the methods provided herein comprise acquiring knowledge that an ILC metastasis comprises an alteration in ARID1A in a sample from an individual having a gastrointestinal ILC metastasis. In some embodiments, the methods provided herein comprise acquiring knowledge that an ILC metastasis comprises an alteration in ARID1A in a sample from an individual having a liver ILC metastasis. In some embodiments, the methods provided herein comprise acquiring knowledge that an ILC metastasis comprises an alteration in ARID1A in a sample from an individual having a skin ILC metastasis. In some embodiments, the methods provided herein comprise detecting an alteration in ARID1A in a sample from an individual having a bone ILC metastasis. In some embodiments, the methods provided herein comprise detecting an alteration in ARID1A in a sample from an individual having a female reproductive system ILC metastasis. In some embodiments, the methods provided herein comprise detecting an alteration in ARID1A in a sample from an individual having a gastrointestinal ILC metastasis. In some embodiments, the methods provided herein comprise detecting an alteration in ARID1A in a sample from an individual having a liver ILC metastasis. In some embodiments, the methods provided herein comprise detecting an alteration in ARID1A in a sample from an individual having a skin ILC metastasis. In some embodiments, the alteration in ARID1A is an inactivating mutation, a loss-of-function mutation, a copy number alteration (e.g., a deletion), a rearrangement, or a gene fusion. In some embodiments, the alteration in ARID1A results in an inactive polypeptide or protein encoded by ARID1A. In some embodiments, the alteration in ARID1A results in a polypeptide or protein encoded by ARID1A that has reduced activity, e.g., as compared to a polypeptide or protein encoded by an ARID1A gene without the alteration. In some embodiments, the alteration in ARID1A results in reduced expression of ARID1A. In some embodiments, the alteration in ARID1A results in loss of expression of ARID1A.

In some embodiments, the methods provided herein comprise acquiring knowledge that an ILC metastasis comprises an alteration in PTEN in a sample from an individual having an ILC metastasis. An exemplary nucleic acid sequence of PTEN is available as Transcript ID NM_000314, provided herein as SEQ ID NO: 13, and available at the website: www[dot]ncbi[dot]nlm[dot]nih[dot]gov/nuccore/NM_000314.

An exemplary amino acid sequence of a PTEN polypeptide is provided herein in SEQ ID NO: 37.

(SEQ ID NO: 37)
MTAIIKEIVSRNKRRYQEDGFDLDLTYIYPNIIAMGFPAERLEGVYRNNI
DDVVRFLDSKHKNHYKIYNLCAERHYDTAKFNCRVAQYPFEDHNPPQLEL
IKPFCEDLDQWLSEDDNHVAAIHCKAGKGRTGVMICAYLLHRGKFLKAQE
ALDFYGEVRTRDKKGVTIPSQRRYVYYYSYLLKNHLDYRPVALLFHKMMF
ETIPMFSGGTCNPQFVVCQLKVKIYSSNSGPTRREDKFMYFEFPQPLPVC
GDIKVEFFHKQNKMLKKDKMFHFWVNTFFIPGPEETSEKVENGSLCDQEI
DSICSIERADNDKEYLVLTLTKNDLDKANKDKANRYFSPNFKVKLYFTKT
VEEPSNPEASSSTSVTPDVSDNEPDHYRYSDTTDSDPENEPFDEDQHTQI
TKV

In some embodiments, acquiring knowledge that the ILC metastasis comprises an alteration in PTEN comprises detecting the alteration in a sample, e.g., in a sample from an ILC tumor, obtained from an individual. In some embodiments, the methods provided herein comprise detecting an alteration in PTEN in a sample from an individual having an ILC metastasis. In some embodiments, the methods further comprise administering an effective amount of an anti-cancer agent responsive to knowledge of the presence of an alteration in PTEN in a sample from an individual having an ILC metastasis. In some embodiments, the methods further comprise providing a report to a party. In some embodiments, the alteration in PTEN is a PTEN deletion. In some embodiments, the ILC metastasis is a bone ILC metastasis, a female reproductive system ILC metastasis, a gastrointestinal ILC metastasis, a liver ILC metastasis, or a skin ILC metastasis. In some embodiments, the methods provided herein comprise acquiring knowledge that an ILC metastasis comprises an alteration in PTEN in a sample from an individual having a bone ILC metastasis. In some embodiments, the methods provided herein comprise acquiring knowledge that an ILC metastasis comprises an alteration in PTEN in a sample from an individual having a female reproductive system ILC metastasis. In some embodiments, the methods provided herein comprise acquiring knowledge that an ILC metastasis comprises an alteration in PTEN in a sample from an individual having a gastrointestinal ILC metastasis. In some embodiments, the methods provided herein comprise acquiring knowledge that an ILC metastasis comprises an alteration in PTEN in a sample from an individual having a liver ILC metastasis. In some embodiments, the methods provided herein comprise acquiring knowledge that an ILC metastasis comprises an alteration in PTEN in a sample from an individual having a skin ILC metastasis. In some embodiments, the methods provided herein comprise detecting an alteration in PTEN in a sample from an individual having a bone ILC metastasis. In some embodiments, the methods provided herein comprise detecting an alteration in PTEN in a sample from an individual having a female reproductive system ILC metastasis. In some embodiments, the methods provided herein comprise detecting an alteration in PTEN in a sample from an individual having a gastrointestinal ILC metastasis. In some embodiments, the methods provided herein comprise detecting an alteration in PTEN in a sample from an individual having a liver ILC metastasis. In some embodiments, the methods provided herein comprise detecting an alteration in PTEN in a sample from an individual having a skin ILC metastasis. In some embodiments, the alteration in PTEN is an inactivating mutation, a loss-of-function mutation, a copy number alteration (e.g., a deletion), a rearrangement, or a gene fusion. In some embodiments, the alteration in PTEN results in an inactive polypeptide or protein encoded by PTEN. In some embodiments, the alteration in PTEN results in a polypeptide or protein encoded by PTEN that has reduced activity, e.g., as compared to a polypeptide or protein encoded by a PTEN gene without the alteration. In some embodiments, the alteration in PTEN results in reduced expression of PTEN. In some embodiments, the alteration in PTEN results in loss of expression of PTEN.

In some embodiments, the methods provided herein comprise acquiring knowledge that an ILC metastasis comprises an alteration in FGFR2 in a sample from an individual having an ILC metastasis. An exemplary nucleic acid sequence of FGFR2 is available as Transcript ID NM_000141, provided herein as SEQ ID NO: 14, and available at the website: www[dot]ncbi[dot]nlm[dot]nih[dot]gov/nuccore/NM_000141.

An exemplary amino acid sequence of a FGFR2 polypeptide is provided herein in SEQ ID NO: 38.

(SEQ ID NO: 38)
MVSWGRFICLVVVTMATLSLARPSFSLVEDTTLEPEEPPTKYQISQPEVY
VAAPGESLEVRCLLKDAAVISWTKDGVHLGPNNRTVLIGEYLQIKGATPR
DSGLYACTASRTVDSETWYFMVNVTDAISSGDDEDDTDGAEDFVSENSNN
KRAPYWTNTEKMEKRLHAVPAANTVKFRCPAGGNPMPTMRWLKNGKEFKQ
EHRIGGYKVRNQHWSLIMESVVPSDKGNYTCVVENEYGSINHTYHLDVVE
RSPHRPILQAGLPANASTVVGGDVEFVCKVYSDAQPHIQWIKHVEKNGSK
YGPDGLPYLKVLKAAGVNTTDKEIEVLYIRNVTFEDAGEYTCLAGNSIGI
SFHSAWLTVLPAPGREKEITASPDYLEIAIYCIGVFLIACMVVTVILCRM
KNTTKKPDFSSQPAVHKLTKRIPLRRQVTVSAESSSSMNSNTPLVRITTR
LSSTADTPMLAGVSEYELPEDPKWEFPRDKLTLGKPLGEGCFGQVVMAEA
VGIDKDKPKEAVTVAVKMLKDDATEKDLSDLVSEMEMMKMIGKHKNIINL
LGACTQDGPLYVIVEYASKGNLREYLRARRPPGMEYSYDINRVPEEQMTF
KDLVSCTYQLARGMEYLASQKCIHRDLAARNVLVTENNVMKIADFGLARD
INNIDYYKKTTNGRLPVKWMAPEALFDRVYTHQSDVWSFGVLMWEIFTLG
GSPYPGIPVEELFKLLKEGHRMDKPANCTNELYMMMRDCWHAVPSQRPTF
KQLVEDLDRILTLTTNEEYLDLSQPLEQYSPSYPDTRSSCSSGDDSVFSP
DPMPYEPCLPQYPHINGSVKT

In some embodiments, acquiring knowledge that the ILC metastasis comprises an alteration in FGFR2 comprises detecting the alteration in a sample, e.g., in a sample from an ILC tumor, obtained from an individual. In some embodiments, the methods provided herein comprise detecting an alteration in FGFR2 in a sample from an individual having an ILC metastasis. In some embodiments, the methods further comprise administering an effective amount of an anti-cancer agent responsive to knowledge of the presence of an alteration in FGFR2 in a sample from an individual having an ILC metastasis. In some embodiments, the methods further comprise providing a report to a party. In some embodiments, the ILC metastasis is a bone ILC metastasis, a female reproductive system ILC metastasis, a gastrointestinal ILC metastasis, a liver ILC metastasis, or a skin ILC metastasis. In some embodiments, the methods provided herein comprise acquiring knowledge that an ILC metastasis comprises an alteration in FGFR2 in a sample from an individual having a bone ILC metastasis. In some embodiments, the methods provided herein comprise acquiring knowledge that an ILC metastasis comprises an alteration in FGFR2 in a sample from an individual having a female reproductive system ILC metastasis. In some embodiments, the methods provided herein comprise acquiring knowledge that an ILC metastasis comprises an alteration in FGFR2 in a sample from an individual having a gastrointestinal ILC metastasis. In some embodiments, the methods provided herein comprise acquiring knowledge that an ILC metastasis comprises an alteration in FGFR2 in a sample from an individual having a liver ILC metastasis. In some embodiments, the methods provided herein comprise acquiring knowledge that an ILC metastasis comprises an alteration in FGFR2 in a sample from an individual having a skin ILC metastasis. In some embodiments, the methods provided herein comprise detecting an alteration in FGFR2 in a sample from an individual having a bone ILC metastasis. In some embodiments, the methods provided herein comprise detecting an alteration in FGFR2 in a sample from an individual having a female reproductive system ILC metastasis. In some embodiments, the methods provided herein comprise detecting an alteration in FGFR2 in a sample from an individual having a gastrointestinal ILC metastasis. In some embodiments, the methods provided herein comprise detecting an alteration in FGFR2 in a sample from an individual having a liver ILC metastasis. In some embodiments, the methods provided herein comprise detecting an alteration in FGFR2 in a sample from an individual having a skin ILC metastasis. In some embodiments, the alteration in FGFR2 is an activating mutation, a gain-of-function mutation, a copy number alteration (e.g., an amplification), a rearrangement, or a gene fusion. In some embodiments, the alteration in FGFR2 results in a constitutively active polypeptide or protein encoded by FGFR2. In some embodiments, the alteration in FGFR2 results in a polypeptide or protein encoded by FGFR2 that has increased activity, e.g., as compared to a polypeptide or protein encoded by an FGFR2 gene without the alteration. In some embodiments, the alteration in FGFR2 results in constitutive expression of FGFR2. In some embodiments, the alteration in FGFR2 results in overexpression of FGFR2. In some embodiments, the alteration in FGFR2 results in one or more amino acid substitutions in a polypeptide encoded by the FGFR2 gene. In some embodiments, the one or more amino acid substitutions are at amino acid position S252, P253, Y375, C382, M391, V395, M537, N549, K659, R664, or any combination thereof, wherein the numbering of the residues is according to SEQ ID NO: 38, or according to the amino acid sequence of a human FGFR2 protein. In some embodiments, the one or more amino acid substitutions in a polypeptide encoded by the FGFR2 gene comprise a S252W, P253R, Y375C, C382R, M391R, V395D, M537I, N549D, N549K, K659M, K659E, K659N, or R664W amino acid substitution, or any combination thereof, wherein the numbering of the residues is according to SEQ ID NO: 38, or according to the amino acid sequence of a human FGFR2 protein. In some embodiments, the alteration results in a frameshift in FGFR2 (e.g., in an mRNA sequence encoded by the FGFR2 gene and in the resulting amino acid sequence). In some embodiments, the frameshift is an S799fs*22 frameshift, wherein the numbering of the residues is according to SEQ ID NO: 38, or according to the amino acid sequence of a human FGFR2 protein. In some embodiments, the S799fs*22 frameshift results from a 2389_2390 insT alteration in an FGFR2 nucleic acid molecule, e.g., an FGFR2 nucleic acid molecule comprising a sequence corresponding to Transcript ID NM_000141, provided herein as SEQ ID NO: 14.

In some embodiments, the methods provided herein comprise acquiring knowledge that an ILC metastasis comprises an alteration in NCOR1 in a sample from an individual having an ILC metastasis. An exemplary nucleic acid sequence of NCOR1 is available as Transcript ID NM_006311, provided herein as SEQ ID NO: 15, and available at the website: www[dot]ncbi[dot]nlm[dot]nih[dot]gov/nuccore/NM_006311.

An exemplary amino acid sequence of a NCOR1 polypeptide is provided herein in SEQ ID NO: 39.

(SEQ ID NO: 39)
MSSSGYPPNQGAFSTEQSRYPPHSVQYTFPNTRHQQEFAVPDYRSSHLEV
SQASQLLQQQQQQQLRRRPSLLSEFHPGSDRPQERRTSYEPFHPGPSPVD
HDSLESKRPRLEQVSDSHFQRVSAAVLPLVHPLPEGLRASADAKKDPAFG
GKHEAPSSPISGQPCGDDQNASPSKLSKEELIQSMDRVDREIAKVEQQIL
KLKKKQQQLEEEAAKPPEPEKPVSPPPVEQKHRSIVQIIYDENRKKAEEA
HKIFEGLGPKVELPLYNQPSDTKVYHENIKTNQVMRKKLILFFKRRNHAR
KQREQKICQRYDQLMEAWEKKVDRIENNPRRKAKESKTREYYEKQFPEIR
KQREQQERFQRVGQRGAGLSATIARSEHEISEIIDGLSEQENNEKQMRQL
SVIPPMMFDAEQRRVKFINMNGLMEDPMKVYKDRQFMNVWTDHEKEIFKD
KFIQHPKNFGLIASYLERKSVPDCVLYYYLTKKNENYKALVRRNYGKRRG
RNQQIARPSQEEKVEEKEEDKAEKTEKKEEEKKDEEEKDEKEDSKENTKE
KDKIDGTAEETEEREQATPRGRKTANSQGRRKGRITRSMTNEAAAASAAA
AAATEEPPPPLPPPPEPISTEPVETSRWTEEEMEVAKKGLVEHGRNWAAI
AKMVGTKSEAQCKNFYFNYKRRHNLDNLLQQHKQKTSRKPREERDVSQCE
SVASTVSAQEDEDIEASNEEENPEDSEVEAVKPSEDSPENATSRGNTEPA
VELEPTTETAPSTSPSLAVPSTKPAEDESVETQVNDSISAETAEQMDVDQ
QEHSAEEGSVCDPPPATKADSVDVEVRVPENHASKVEGDNTKERDLDRAS
EKVEPRDEDLVVAQQINAQRPEPQSDNDSSATCSADEDVDGEPERQRMFP
MDSKPSLLNPTGSILVSSPLKPNPLDLPQLQHRAAVIPPMVSCTPCNIPI
GTPVSGYALYQRHIKAMHESALLEEQRQRQEQIDLECRSSTSPCGTSKSP
NREWEVLQPAPHQVITNLPEGVRLPTTRPTRPPPPLIPSSKTTVASEKPS
FIMGGSISQGTPGTYLTSHNQASYTQETPKPSVGSISLGLPRQQESAKSA
TLPYIKQEEFSPRSQNSQPEGLLVRAQHEGVVRGTAGAIQEGSITRGTPT
SKISVESIPSLRGSITQGTPALPQTGIPTEALVKGSISRMPIEDSSPEKG
REEAASKGHVIYEGKSGHILSYDNIKNAREGTRSPRTAHEISLKRSYESV
EGNIKQGMSMRESPVSAPLEGLICRALPRGSPHSDLKERTVLSGSIMQGT
PRATTESFEDGLKYPKQIKRESPPIRAFEGAITKGKPYDGITTIKEMGRS
IHEIPRQDILTQESRKTPEVVQSTRPIIEGSISQGTPIKFDNNSGQSAIK
HNVKSLITGPSKLSRGMPPLEIVPENIKVVERGKYEDVKAGETVRSRHTS
VVSSGPSVLRSTLHEAPKAQLSPGIYDDTSARRTPVSYQNTMSRGSPMMN
RTSDVTISSNKSTNHERKSTLTPTQRESIPAKSPVPGVDPVVSHSPFDPH
HRGSTAGEVYRSHLPTHLDPAMPFHRALDPAAAAYLFQRQLSPTPGYPSQ
YQLYAMENTRQTILNDYITSQQMQVNLRPDVARGLSPREQPLGLPYPATR
GIIDLTNMPPTILVPHPGGTSTPPMDRITYIPGTQITFPPRPYNSASMSP
GHPTHLAAAASAEREREREREKERERERIAAASSDLYLRPGSEQPGRPGS
HGYVRSPSPSVRTQETMLQQRPSVFQGTNGTSVITPLDPTAQLRIMPLPA
GGPSISQGLPASRYNTAADALAALVDAAASAPQMDVSKTKESKHEAARLE
ENLRSRSAAVSEQQQLEQKTLEVEKRSVQCLYTSSAFPSGKPQPHSSVVY
SEAGKDKGPPPKSRYEEELRTRGKTTITAANFIDVIITRQIASDKDARER
GSQSSDSSSSLSSHRYETPSDAIEVISPASSPAPPQEKLQTYQPEVVKAN
QAENDPTRQYEGPLHHYRPQQESPSPQQQLPPSSQAEGMGQVPRTHRLIT
LADHICQIITQDFARNQVSSQTPQQPPTSTFQNSPSALVSTPVRTKTSNR
YSPESQAQSVHHQRPGSRVSPENLVDKSRGSRPGKSPERSHVSSEPYEPI
SPPQVPVVHEKQDSLLLLSQRGAEPAEQRNDARSPGSISYLPSFFTKLEN
TSPMVKSKKQEIFRKLNSSGGGDSDMAAAQPGTEIFNLPAVTTSGSVSSR
GHSFADPASNLGLEDIIRKALMGSFDDKVEDHGVVMSQPMGVVPGTANTS
VVTSGETRREEGDPSPHSGGVCKPKLISKSNSRKSKSPIPGQGYLGTERP
SSVSSVHSEGDYHRQTPGWAWEDRPSSTGSTQFPYNPLTMRMLSSTPPTP
IACAPSAVNQAAPHQQNRIWEREPAPLLSAQYETLSDSDD

In some embodiments, acquiring knowledge that the ILC metastasis comprises an alteration in NCOR1 comprises detecting the alteration in a sample, e.g., in a sample from an ILC tumor, obtained from an individual. In some embodiments, the methods provided herein comprise detecting an alteration in NCOR1 in a sample from an individual having an ILC metastasis. In some embodiments, the methods further comprise administering an effective amount of an anti-cancer agent responsive to knowledge of the presence of an alteration in NCOR1 in a sample from an individual having an ILC metastasis. In some embodiments, the methods further comprise providing a report to a party. In some embodiments, the alteration comprises one or more of a substitution of one or more nucleotides, an insertion of one or more nucleotides, or a deletion of one or more nucleotides in NCOR1. In some embodiments, the ILC metastasis is a bone ILC metastasis, a female reproductive system ILC metastasis, a gastrointestinal ILC metastasis, a liver ILC metastasis, or a skin ILC metastasis. In some embodiments, the methods provided herein comprise acquiring knowledge that an ILC metastasis comprises an alteration in NCOR1 in a sample from an individual having a bone ILC metastasis. In some embodiments, the methods provided herein comprise acquiring knowledge that an ILC metastasis comprises an alteration in NCOR1 in a sample from an individual having a female reproductive system ILC metastasis. In some embodiments, the methods provided herein comprise acquiring knowledge that an ILC metastasis comprises an alteration in NCOR1 in a sample from an individual having a gastrointestinal ILC metastasis. In some embodiments, the methods provided herein comprise acquiring knowledge that an ILC metastasis comprises an alteration in NCOR1 in a sample from an individual having a liver ILC metastasis. In some embodiments, the methods provided herein comprise acquiring knowledge that an ILC metastasis comprises an alteration in NCOR1 in a sample from an individual having a skin ILC metastasis. In some embodiments, the methods provided herein comprise detecting an alteration in NCOR1 in a sample from an individual having a bone ILC metastasis. In some embodiments, the methods provided herein comprise detecting an alteration in NCOR1 in a sample from an individual having a female reproductive system ILC metastasis. In some embodiments, the methods provided herein comprise detecting an alteration in NCOR1 in a sample from an individual having a gastrointestinal ILC metastasis. In some embodiments, the methods provided herein comprise detecting an alteration in NCOR1 in a sample from an individual having a liver ILC metastasis. In some embodiments, the methods provided herein comprise detecting an alteration in NCOR1 in a sample from an individual having a skin ILC metastasis. In some embodiments, the alteration in NCOR1 is an inactivating mutation, a loss-of-function mutation, a copy number alteration (e.g., a deletion), a rearrangement, or a gene fusion. In some embodiments, the alteration in NCOR1 results in an inactive polypeptide or protein encoded by NCOR1. In some embodiments, the alteration in NCOR1 results in a polypeptide or protein encoded by NCOR1 that has reduced activity, e.g., as compared to a polypeptide or protein encoded by an NCOR1 gene without the alteration. In some embodiments, the alteration in NCOR1 results in reduced expression of NCOR1. In some embodiments, the alteration in NCOR1 results in loss of expression of NCOR1.

In some embodiments, the methods provided herein comprise acquiring knowledge that an ILC metastasis comprises an alteration in SMAD4 in a sample from an individual having an ILC metastasis. An exemplary nucleic acid sequence of SMAD4 is available as Transcript ID NM_005359, provided herein as SEQ ID NO: 16, and available at the website: www[dot]ncbi[dot]nlm[dot]nih[dot]gov/nuccore/NM_005359.

An exemplary amino acid sequence of a SMAD4 polypeptide is provided herein in SEQ ID NO: 40.

(SEQ ID NO: 40)
MDNMSITNTPTSNDACLSIVHSLMCHRQGGESETFAKRAIESLVKKLKEK
KDELDSLITAITTNGAHPSKCVTIQRTLDGRLQVAGRKGFPHVIYARLWR
WPDLHKNELKHVKYCQYAFDLKCDSVCVNPYHYERVVSPGIDLSGLTLQS
NAPSSMMVKDEYVHDFEGQPSLSTEGHSIQTIQHPPSNRASTETYSTPAL
LAPSESNATSTANFPNIPVASTSQPASILGGSHSEGLLQIASGPQPGQQQ
NGFTGQPATYHHNSTTTWTGSRTAPYTPNLPHHQNGHLQHHPPMPPHPGH
YWPVHNELAFQPPISNHPAPEYWCSIAYFEMDVQVGETFKVPSSCPIVTV
DGYVDPSGGDRFCLGQLSNVHRTEAIERARLHIGKGVQLECKGEGDVWVR
CLSDHAVFVQSYYLDREAGRAPGDAVHKIYPSAYIKVFDLRQCHRQMQQQ
AATAQAAAAAQAAAVAGNIPGPGSVGGIAPAISLSAAAGIGVDDLRRLCI
LRMSFVKGWGPDYPRQSIKETPCWIEIHLHRALQLLDEVLHTMPIADPQP
LD

In some embodiments, acquiring knowledge that the ILC metastasis comprises an alteration in SMAD4 comprises detecting the alteration in a sample, e.g., in a sample from an ILC tumor, obtained from an individual. In some embodiments, the methods provided herein comprise detecting an alteration in SMAD4 in a sample from an individual having an ILC metastasis. In some embodiments, the methods further comprise administering an effective amount of an anti-cancer agent responsive to knowledge of the presence of an alteration in SMAD4 in a sample from an individual having an ILC metastasis. In some embodiments, the methods further comprise providing a report to a party. In some embodiments, the ILC metastasis is a bone ILC metastasis, a female reproductive system ILC metastasis, a gastrointestinal ILC metastasis, a liver ILC metastasis, or a skin ILC metastasis. In some embodiments, the methods provided herein comprise acquiring knowledge that an ILC metastasis comprises an alteration in SMAD4 in a sample from an individual having a bone ILC metastasis. In some embodiments, the methods provided herein comprise acquiring knowledge that an ILC metastasis comprises an alteration in SMAD4 in a sample from an individual having a female reproductive system ILC metastasis. In some embodiments, the methods provided herein comprise acquiring knowledge that an ILC metastasis comprises an alteration in SMAD4 in a sample from an individual having a gastrointestinal ILC metastasis. In some embodiments, the methods provided herein comprise acquiring knowledge that an ILC metastasis comprises an alteration in SMAD4 in a sample from an individual having a liver ILC metastasis. In some embodiments, the methods provided herein comprise acquiring knowledge that an ILC metastasis comprises an alteration in SMAD4 in a sample from an individual having a skin ILC metastasis. In some embodiments, the methods provided herein comprise detecting an alteration in SMAD4 in a sample from an individual having a bone ILC metastasis. In some embodiments, the methods provided herein comprise detecting an alteration in SMAD4 in a sample from an individual having a female reproductive system ILC metastasis. In some embodiments, the methods provided herein comprise detecting an alteration in SMAD4 in a sample from an individual having a gastrointestinal ILC metastasis. In some embodiments, the methods provided herein comprise detecting an alteration in SMAD4 in a sample from an individual having a liver ILC metastasis. In some embodiments, the methods provided herein comprise detecting an alteration in SMAD4 in a sample from an individual having a skin ILC metastasis. In some embodiments, the alteration in SMAD4 is an inactivating mutation, a loss-of-function mutation, a copy number alteration (e.g., a deletion), a rearrangement, or a gene fusion. In some embodiments, the alteration in SMAD4 results in an inactive polypeptide or protein encoded by SMAD4. In some embodiments, the alteration in SMAD4 results in a polypeptide or protein encoded by SMAD4 that has reduced activity, e.g., as compared to a polypeptide or protein encoded by a SMAD4 gene without the alteration. In some embodiments, the alteration in SMAD4 results in reduced expression of SMAD4. In some embodiments, the alteration in SMAD4 results in loss of expression of SMAD4.

In some embodiments, the methods provided herein comprise acquiring knowledge that an ILC metastasis comprises an alteration in FOXP1 in a sample from an individual having an ILC metastasis. An exemplary nucleic acid sequence of FOXP1 is available as Transcript ID NM_032682, provided herein as SEQ ID NO: 17, and available at the website: www[dot]ncbi[dot]nlm[dot]nih[dot]gov/nuccore/NM_032682.

An exemplary amino acid sequence of a FOXP1 polypeptide is provided herein in SEQ ID NO: 41.

(SEQ ID NO: 41)
MMQESGTETKSNGSAIQNGSGGSNHLLECGGLREGRSNGETPAVDIGAAD
LAHAQQQQQQALQVARQLLLQQQQQQQVSGLKSPKRNDKQPALQVPVSVA
MMTPQVITPQQMQQILQQQVLSPQQLQVLLQQQQALMLQQQQLQEFYKKQ
QEQLQLQLLQQQHAGKQPKEQQQVATQQLAFQQQLLQMQQLQQQHLLSLQ
RQGLLTIQPGQPALPLQPLAQGMIPTELQQLWKEVTSAHTAEETTGNNHS
SLDLTTTCVSSSAPSKTSLIMNPHASTNGQLSVHTPKRESLSHEEHPHSH
PLYGHGVCKWPGCEAVCEDFQSFLKHLNSEHALDDRSTAQCRVQMQVVQQ
LELQLAKDKERLQAMMTHLHVKSTEPKAAPQPLNLVSSVTLSKSASEASP
QSLPHTPTTPTAPLTPVTQGPSVITTTSMHTVGPIRRRYSDKYNVPISSA
DIAQNQEFYKNAEVRPPFTYASLIRQAILESPEKQLTLNEIYNWFTRMFA
YFRRNAATWKNAVRHNLSLHKCFVRVENVKGAVWTVDEVEFQKRRPQKIS
GNPSLIKNMQSSHAYCTPLNAALQASMAENSIPLYTTASMGNPTLGNLAS
AIREELNGAMEHTNSNESDSSPGRSPMQAVHPVHVKEEPLDPEEAEGPLS
LVTTANHSPDFDHDRDYEDEPVNEDME

In some embodiments, acquiring knowledge that the ILC metastasis comprises an alteration in FOXP1 comprises detecting the alteration in a sample, e.g., in a sample from an ILC tumor, obtained from an individual. In some embodiments, the methods provided herein comprise detecting an alteration in FOXP1 in a sample from an individual having an ILC metastasis. In some embodiments, the methods further comprise administering an effective amount of an anti-cancer agent responsive to knowledge of the presence of an alteration in FOXP1 in a sample from an individual having an ILC metastasis. In some embodiments, the methods further comprise providing a report to a party. In some embodiments, the alteration comprises one or more of a substitution of one or more nucleotides, an insertion of one or more nucleotides, or a deletion of one or more nucleotides in FOXP1. In some embodiments, the ILC metastasis is a bone ILC metastasis, a female reproductive system ILC metastasis, a gastrointestinal ILC metastasis, a liver ILC metastasis, or a skin ILC metastasis. In some embodiments, the methods provided herein comprise acquiring knowledge that an ILC metastasis comprises an alteration in FOXP1 in a sample from an individual having a bone ILC metastasis. In some embodiments, the methods provided herein comprise acquiring knowledge that an ILC metastasis comprises an alteration in FOXP1 in a sample from an individual having a female reproductive system ILC metastasis. In some embodiments, the methods provided herein comprise acquiring knowledge that an ILC metastasis comprises an alteration in FOXP1 in a sample from an individual having a gastrointestinal ILC metastasis. In some embodiments, the methods provided herein comprise acquiring knowledge that an ILC metastasis comprises an alteration in FOXP1 in a sample from an individual having a liver ILC metastasis. In some embodiments, the methods provided herein comprise acquiring knowledge that an ILC metastasis comprises an alteration in FOXP1 in a sample from an individual having a skin ILC metastasis. In some embodiments, the methods provided herein comprise detecting an alteration in FOXP1 in a sample from an individual having a bone ILC metastasis. In some embodiments, the methods provided herein comprise detecting an alteration in FOXP1 in a sample from an individual having a female reproductive system ILC metastasis. In some embodiments, the methods provided herein comprise detecting an alteration in FOXP1 in a sample from an individual having a gastrointestinal ILC metastasis. In some embodiments, the methods provided herein comprise detecting an alteration in FOXP1 in a sample from an individual having a liver ILC metastasis. In some embodiments, the methods provided herein comprise detecting an alteration in FOXP1 in a sample from an individual having a skin ILC metastasis. In some embodiments, the alteration in FOXP1 is an inactivating mutation, a loss-of-function mutation, a copy number alteration (e.g., a deletion), a rearrangement, or a gene fusion. In some embodiments, the alteration in FOXP1 results in an inactive polypeptide or protein encoded by FOXP1. In some embodiments, the alteration in FOXP1 results in a polypeptide or protein encoded by FOXP1 that has reduced activity, e.g., as compared to a polypeptide or protein encoded by a FOXP1 gene without the alteration. In some embodiments, the alteration in FOXP1 results in reduced expression of FOXP1. In some embodiments, the alteration in FOXP1 results in loss of expression of FOXP1.

In some embodiments, the methods provided herein comprise acquiring knowledge that an ILC metastasis comprises an alteration in APC in a sample from an individual having an ILC metastasis. An exemplary nucleic acid sequence of APC is available as Transcript ID NM_000038, provided herein as SEQ ID NO: 18, and available at the website: www[dot]ncbi[dot]nlm[dot]nih[dot]gov/nuccore/NM_000038.

An exemplary amino acid sequence of an APC polypeptide is provided herein in SEQ ID NO: 42.

(SEQ ID NO: 42)
MAAASYDQLLKQVEALKMENSNLRQELEDNSNHLTKLETEASNMKEVLKQ
LQGSIEDEAMASSGQIDLLERLKELNLDSSNFPGVKLRSKMSLRSYGSRE
GSVSSRSGECSPVPMGSFPRRGFVNGSRESTGYLEELEKERSLLLADLDK
EEKEKDWYYAQLQNLTKRIDSLPLTENFSLQTDMTRRQLEYEARQIRVAM
EEQLGTCQDMEKRAQRRIARIQQIEKDILRIRQLLQSQATEAERSSQNKH
ETGSHDAERQNEGQGVGEINMATSGNGQGSTTRMDHETASVLSSSSTHSA
PRRLTSHLGTKVEMVYSLLSMLGTHDKDDMSRTLLAMSSSQDSCISMRQS
GCLPLLIQLLHGNDKDSVLLGNSRGSKEARARASAALHNIIHSQPDDKRG
RREIRVLHLLEQIRAYCETCWEWQEAHEPGMDQDKNPMPAPVEHQICPAV
CVLMKLSFDEEHRHAMNELGGLQAIAELLQVDCEMYGLTNDHYSITLRRY
AGMALTNLTFGDVANKATLCSMKGCMRALVAQLKSESEDLQQVIASVLRN
LSWRADVNSKKTLREVGSVKALMECALEVKKESTLKSVLSALWNLSAHCT
ENKADICAVDGALAFLVGTLTYRSQTNTLAIIESGGGILRNVSSLIATNE
DHRQILRENNCLQTLLQHLKSHSLTIVSNACGTLWNLSARNPKDQEALWD
MGAVSMLKNLIHSKHKMIAMGSAAALRNLMANRPAKYKDANIMSPGSSLP
SLHVRKQKALEAELDAQHLSETFDNIDNLSPKASHRSKQRHKQSLYGDYV
FDTNRHDDNRSDNFNTGNMTVLSPYLNTTVLPSSSSSRGSLDSSRSEKDR
SLERERGIGLGNYHPATENPGTSSKRGLQISTTAAQIAKVMEEVSAIHTS
QEDRSSGSTTELHCVTDERNALRRSSAAHTHSNTYNFTKSENSNRTCSMP
YAKLEYKRSSNDSLNSVSSSDGYGKRGQMKPSIESYSEDDESKFCSYGQY
PADLAHKIHSANHMDDNDGELDTPINYSLKYSDEQLNSGRQSPSQNERWA
RPKHIIEDEIKQSEQRQSRNQSTTYPVYTESTDDKHLKFQPHFGQQECVS
PYRSRGANGSETNRVGSNHGINQNVSQSLCQEDDYEDDKPTNYSERYSEE
EQHEEEERPTNYSIKYNEEKRHVDQPIDYSLKYATDIPSSQKQSFSFSKS
SSGQSSKTEHMSSSSENTSTPSSNAKRQNQLHPSSAQSRSGQPQKAATCK
VSSINQETIQTYCVEDTPICFSRCSSLSSLSSAEDEIGCNQTTQEADSAN
TLQIAEIKEKIGTRSAEDPVSEVPAVSQHPRTKSSRLQGSSLSSESARHK
AVEFSSGAKSPSKSGAQTPKSPPEHYVQETPLMFSRCTSVSSLDSFESRS
IASSVQSEPCSGMVSGIISPSDLPDSPGQTMPPSRSKTPPPPPQTAQTKR
EVPKNKAPTAEKRESGPKQAAVNAAVQRVQVLPDADTLLHFATESTPDGF
SCSSSLSALSLDEPFIQKDVELRIMPPVQENDNGNETESEQPKESNENQE
KEAEKTIDSEKDLLDDSDDDDIEILEECIISAMPTKSSRKAKKPAQTASK
LPPPVARKPSQLPVYKLLPSQNRLQPQKHVSFTPGDDMPRVYCVEGTPIN
FSTATSLSDLTIESPPNELAAGEGVRGGAQSGEFEKRDTIPTEGRSTDEA
QGGKTSSVTIPELDDNKAEEGDILAECINSAMPKGKSHKPFRVKKIMDQV
QQASASSSAPNKNQLDGKKKKPTSPVKPIPQNTEYRTRVRKNADSKNNLN
AERVFSDNKDSKKQNLKNNSKVFNDKLPNNEDRVRGSFAFDSPHHYTPIE
GTPYCFSRNDSLSSLDFDDDDVDLSREKAELRKAKENKESEAKVTSHTEL
TSNQQSANKTQAIAKQPINRGQPKPILQKQSTFPQSSKDIPDRGAATDEK
LQNFAIENTPVCFSHNSSLSSLSDIDQENNNKENEPIKETEPPDSQGEPS
KPQASGYAPKSFHVEDTPVCFSRNSSLSSLSIDSEDDLLQECISSAMPKK
KKPSRLKGDNEKHSPRNMGGILGEDLTLDLKDIQRPDSEHGLSPDSENFD
WKAIQEGANSIVSSLHQAAAAACLSRQASSDSDSILSLKSGISLGSPFHL
TPDQEEKPFTSNKGPRILKPGEKSTLETKKIESESKGIKGGKKVYKSLIT
GKVRSNSEISGQMKQPLQANMPSISRGRTMIHIPGVRNSSSSTSPVSKKG
PPLKTPASKSPSEGQTATTSPRGAKPSVKSELSPVARQTSQIGGSSKAPS
RSGSRDSTPSRPAQQPLSRPIQSPGRNSISPGRNGISPPNKLSQLPRTSS
PSTASTKSSGSGKMSYTSPGRQMSQQNLTKQTGLSKNASSIPRSESASKG
LNQMNNGNGANKKVELSRMSSTKSSGSESDRSERPVLVRQSTFIKEAPSP
TLRRKLEESASFESLSPSSRPASPTRSQAQTPVLSPSLPDMSLSTHSSVQ
AGGWRKLPPNLSPTIEYNDGRPAKRHDIARSHSESPSRLPINRSGTWKRE
HSKHSSSLPRVSTWRRTGSSSSILSASSESSEKAKSEDEKHVNSISGTKQ
SKENQVSAKGTWRKIKENEFSPTNSTSQTVSSGATNGAESKTLIYQMAPA
VSKTEDVWVRIEDCPINNPRSGRSPTGNTPPVIDSVSEKANPNIKDSKDN
QAKQNVGNGSVPMRTVGLENRLNSFIQVDAPDQKGTEIKPGQNNPVPVSE
TNESSIVERTPFSSSSSSKHSSPSGTVAARVTPFNYNPSPRKSSADSTSA
RPSQIPTPVNNNTKKRDSKTDSTESSGTQSPKRHSGSYLVTSV

In some embodiments, acquiring knowledge that the ILC metastasis comprises an alteration in APC comprises detecting the alteration in a sample, e.g., in a sample from an ILC tumor, obtained from an individual. In some embodiments, the methods provided herein comprise detecting an alteration in APC in a sample from an individual having an ILC metastasis. In some embodiments, the methods further comprise administering an effective amount of an anti-cancer agent responsive to knowledge of the presence of an alteration in APC in a sample from an individual having an ILC metastasis. In some embodiments, the methods further comprise providing a report to a party. In some embodiments, the alteration comprises one or more of a substitution of one or more nucleotides, an insertion of one or more nucleotides, or a deletion of one or more nucleotides in APC. In some embodiments, the ILC metastasis is a bone ILC metastasis, a female reproductive system ILC metastasis, a gastrointestinal ILC metastasis, a liver ILC metastasis, or a skin ILC metastasis. In some embodiments, the methods provided herein comprise acquiring knowledge that an ILC metastasis comprises an alteration in APC in a sample from an individual having a bone ILC metastasis. In some embodiments, the methods provided herein comprise acquiring knowledge that an ILC metastasis comprises an alteration in APC in a sample from an individual having a female reproductive system ILC metastasis. In some embodiments, the methods provided herein comprise acquiring knowledge that an ILC metastasis comprises an alteration in APC in a sample from an individual having a gastrointestinal ILC metastasis. In some embodiments, the methods provided herein comprise acquiring knowledge that an ILC metastasis comprises an alteration in APC in a sample from an individual having a liver ILC metastasis. In some embodiments, the methods provided herein comprise acquiring knowledge that an ILC metastasis comprises an alteration in APC in a sample from an individual having a skin ILC metastasis. In some embodiments, the methods provided herein comprise detecting an alteration in APC in a sample from an individual having a bone ILC metastasis. In some embodiments, the methods provided herein comprise detecting an alteration in APC in a sample from an individual having a female reproductive system ILC metastasis. In some embodiments, the methods provided herein comprise detecting an alteration in APC in a sample from an individual having a gastrointestinal ILC metastasis. In some embodiments, the methods provided herein comprise detecting an alteration in APC in a sample from an individual having a liver ILC metastasis. In some embodiments, the methods provided herein comprise detecting an alteration in APC in a sample from an individual having a skin ILC metastasis. In some embodiments, the alteration in APC is an inactivating mutation, a loss-of-function mutation, a copy number alteration (e.g., a deletion), a rearrangement, or a gene fusion. In some embodiments, the alteration in APC results in an inactive polypeptide or protein encoded by APC. In some embodiments, the alteration in APC results in a polypeptide or protein encoded by APC that has reduced activity, e.g., as compared to a polypeptide or protein encoded by an APC gene without the alteration. In some embodiments, the alteration in APC results in reduced expression of APC. In some embodiments, the alteration in APC results in loss of expression of APC.

In some embodiments, the methods provided herein comprise acquiring knowledge that an ILC metastasis comprises an alteration in SOX9 in a sample from an individual having an ILC metastasis. An exemplary nucleic acid sequence of SOX9 is available as Transcript ID NM_000346, provided herein as SEQ ID NO: 19, and available at the website: www[dot]ncbi[dot]nlm[dot]nih[dot]gov/nuccore/NM_000346.

An exemplary amino acid sequence of a SOX9 polypeptide is provided herein in SEQ ID NO: 43.

(SEQ ID NO: 43)
MNLLDPFMKMTDEQEKGLSGAPSPTMSEDSAGSPCPSGSGSDTENTRPQE
NTFPKGEPDLKKESEEDKFPVCIREAVSQVLKGYDWTLVPMPVRVNGSSK
NKPHVKRPMNAFMVWAQAARRKLADQYPHLHNAELSKTLGKLWRLLNESE
KRPFVEEAERLRVQHKKDHPDYKYQPRRRKSVKNGQAEAEEATEQTHISP
NAIFKALQADSPHSSSGMSEVHSPGEHSGQSQGPPTPPTTPKTDVQPGKA
DLKREGRPLPEGGRQPPIDFRDVDIGELSSDVISNIETFDVNEFDQYLPP
NGHPGVPATHGQVTYTGSYGISSTAATPASAGHVWMSKQQAPPPPPQQPP
QAPPAPQAPPQPQAAPPQQPAAPPQQPQAHTLTTLSSEPGQSQRTHIKTE
QLSPSHYSEQQQHSPQQIAYSPFNLPHYSPSYPPITRSQYDYTDHQNSSS
YYSHAAGQGTGLYSTFTYMNPAQRPMYTPIADTSGVPSIPQTHSPQHWEQ
PVYTQLTRP

In some embodiments, acquiring knowledge that the ILC metastasis comprises an alteration in SOX9 comprises detecting the alteration in a sample, e.g., in a sample from an ILC tumor, obtained from an individual. In some embodiments, the methods provided herein comprise detecting an alteration in SOX9 in a sample from an individual having an ILC metastasis. In some embodiments, the methods further comprise administering an effective amount of an anti-cancer agent responsive to knowledge of the presence of an alteration in SOX9 in a sample from an individual having an ILC metastasis. In some embodiments, the methods further comprise providing a report to a party. In some embodiments, the ILC metastasis is a bone ILC metastasis, a female reproductive system ILC metastasis, a gastrointestinal ILC metastasis, a liver ILC metastasis, or a skin ILC metastasis. In some embodiments, the methods provided herein comprise acquiring knowledge that an ILC metastasis comprises an alteration in SOX9 in a sample from an individual having a bone ILC metastasis. In some embodiments, the methods provided herein comprise acquiring knowledge that an ILC metastasis comprises an alteration in SOX9 in a sample from an individual having a female reproductive system ILC metastasis. In some embodiments, the methods provided herein comprise acquiring knowledge that an ILC metastasis comprises an alteration in SOX9 in a sample from an individual having a gastrointestinal ILC metastasis. In some embodiments, the methods provided herein comprise acquiring knowledge that an ILC metastasis comprises an alteration in SOX9 in a sample from an individual having a liver ILC metastasis. In some embodiments, the methods provided herein comprise acquiring knowledge that an ILC metastasis comprises an alteration in SOX9 in a sample from an individual having a skin ILC metastasis. In some embodiments, the methods provided herein comprise detecting an alteration in SOX9 in a sample from an individual having a bone ILC metastasis. In some embodiments, the methods provided herein comprise detecting an alteration in SOX9 in a sample from an individual having a female reproductive system ILC metastasis. In some embodiments, the methods provided herein comprise detecting an alteration in SOX9 in a sample from an individual having a gastrointestinal ILC metastasis. In some embodiments, the methods provided herein comprise detecting an alteration in SOX9 in a sample from an individual having a liver ILC metastasis. In some embodiments, the methods provided herein comprise detecting an alteration in SOX9 in a sample from an individual having a skin ILC metastasis. In some embodiments, the alteration in SOX9 is an inactivating mutation, a loss-of-function mutation, a copy number alteration (e.g., a deletion), a rearrangement, or a gene fusion. In some embodiments, the alteration in SOX9 results in an inactive polypeptide or protein encoded by SOX9. In some embodiments, the alteration in SOX9 results in a polypeptide or protein encoded by SOX9 that has reduced activity, e.g., as compared to a polypeptide or protein encoded by a SOX9 gene without the alteration. In some embodiments, the alteration in SOX9 results in reduced expression of SOX9. In some embodiments, the alteration in SOX9 results in loss of expression of SOX9.

In some embodiments, the methods provided herein comprise acquiring knowledge that an ILC metastasis comprises an alteration in CASP8 in a sample from an individual having an ILC metastasis. An exemplary nucleic acid sequence of CASP8 is available as Transcript ID NM_001228, provided herein as SEQ ID NO: 20, and available at the website: www[dot]ncbi[dot]nlm[dot]nih[dot]gov/nuccore/NM_001228.

An exemplary amino acid sequence of a CASP8 polypeptide is provided herein in SEQ ID NO: 44.

(SEQ ID NO: 44)
MDFSRNLYDIGEQLDSEDLASLKFLSLDYIPQRKQEPIKDALMLFQRLQE
KRMLEESNLSFLKELLFRINRLDLLITYLNTRKEEMERELQTPGRAQISA
YRFHFCRMSWAEANSQCQTQSVPFWRRVDHLLIRVMLYQISEEVSRSELR
SFKELLQEEISKCKLDDDMNLLDIFIEMEKRVILGEGKLDILKRVCAQIN
KSLLKIINDYEEFSKGEELCGVMTISDSPREQDSESQTLDKVYQMKSKPR
GYCLIINNHNFAKAREKVPKLHSIRDRNGTHLDAGALTTTFEELHFEIKP
HDDCTVEQIYEILKIYQLMDHSNMDCFICCILSHGDKGIIYGTDGQEAPI
YELTSQFTGLKCPSLAGKPKVFFIQACQGDNYQKGIPVETDSEEQPYLEM
DLSSPQTRYIPDEADFLLGMATVNNCVSYRNPAEGTWYIQSLCQSLRERC
PRGDDILTILTEVNYEVSNKDDKKNMGKQMPQPTFTLRKKLVFPSD

In some embodiments, acquiring knowledge that the ILC metastasis comprises an alteration in CASP8 comprises detecting the alteration in a sample, e.g., in a sample from an ILC tumor, obtained from an individual. In some embodiments, the methods provided herein comprise detecting an alteration in CASP8 in a sample from an individual having an ILC metastasis. In some embodiments, the methods further comprise administering an effective amount of an anti-cancer agent responsive to knowledge of the presence of an alteration in CASP8 in a sample from an individual having an ILC metastasis. In some embodiments, the methods further comprise providing a report to a party. In some embodiments, the alteration comprises one or more of a substitution of one or more nucleotides, an insertion of one or more nucleotides, or a deletion of one or more nucleotides in CASP8. In some embodiments, the ILC metastasis is a bone ILC metastasis, a female reproductive system ILC metastasis, a gastrointestinal ILC metastasis, a liver ILC metastasis, or a skin ILC metastasis. In some embodiments, the methods provided herein comprise acquiring knowledge that an ILC metastasis comprises an alteration in CASP8 in a sample from an individual having a bone ILC metastasis. In some embodiments, the methods provided herein comprise acquiring knowledge that an ILC metastasis comprises an alteration in CASP8 in a sample from an individual having a female reproductive system ILC metastasis. In some embodiments, the methods provided herein comprise acquiring knowledge that an ILC metastasis comprises an alteration in CASP8 in a sample from an individual having a gastrointestinal ILC metastasis. In some embodiments, the methods provided herein comprise acquiring knowledge that an ILC metastasis comprises an alteration in CASP8 in a sample from an individual having a liver ILC metastasis. In some embodiments, the methods provided herein comprise acquiring knowledge that an ILC metastasis comprises an alteration in CASP8 in a sample from an individual having a skin ILC metastasis. In some embodiments, the methods provided herein comprise detecting an alteration in CASP8 in a sample from an individual having a bone ILC metastasis. In some embodiments, the methods provided herein comprise detecting an alteration in CASP8 in a sample from an individual having a female reproductive system ILC metastasis. In some embodiments, the methods provided herein comprise detecting an alteration in CASP8 in a sample from an individual having a gastrointestinal ILC metastasis. In some embodiments, the methods provided herein comprise detecting an alteration in CASP8 in a sample from an individual having a liver ILC metastasis. In some embodiments, the methods provided herein comprise detecting an alteration in CASP8 in a sample from an individual having a skin ILC metastasis. In some embodiments, the alteration in CASP8 is an inactivating mutation, a loss-of-function mutation, a copy number alteration (e.g., a deletion), a rearrangement, or a gene fusion. In some embodiments, the alteration in CASP8 results in an inactive polypeptide or protein encoded by CASP8. In some embodiments, the alteration in CASP8 results in a polypeptide or protein encoded by CASP8 that has reduced activity, e.g., as compared to a polypeptide or protein encoded by a CASP8 gene without the alteration. In some embodiments, the alteration in CASP8 results in reduced expression of CASP8. In some embodiments, the alteration in CASP8 results in loss of expression of CASP8.

In some embodiments, the methods provided herein comprise acquiring knowledge that an ILC metastasis comprises an alteration in PTPN11 in a sample from an individual having an ILC metastasis. An exemplary nucleic acid sequence of PTPN11 is available as Transcript ID NM_002834, provided herein as SEQ ID NO: 21, and available at the website: www[dot]ncbi[dot]nlm[dot]nih[dot]gov/nuccore/NM_002834.

An exemplary amino acid sequence of a PTPN11 polypeptide is provided herein in SEQ ID NO: 45.

(SEQ ID NO: 45)
MTSRRWFHPNITGVEAENLLLTRGVDGSFLARPSKSNPGDFTLSVRRNGA
VTHIKIQNTGDYYDLYGGEKFATLAELVQYYMEHHGQLKEKNGDVIELKY
PLNCADPTSERWFHGHLSGKEAEKLLTEKGKHGSFLVRESQSHPGDFVLS
VRTGDDKGESNDGKSKVTHVMIRCQELKYDVGGGERFDSLTDLVEHYKKN
PMVETLGTVLQLKQPLNTTRINAAEIESRVRELSKLAETTDKVKQGFWEE
FETLQQQECKLLYSRKEGQRQENKNKNRYKNILPFDHTRVVLHDGDPNEP
VSDYINANIIMPEFETKCNNSKPKKSYIATQGCLQNTVNDFWRMVFQENS
RVIVMTTKEVERGKSKCVKYWPDEYALKEYGVMRVRNVKESAAHDYTLRE
LKLSKVGQGNTERTVWQYHFRTWPDHGVPSDPGGVLDFLEEVHHKQESIM
DAGPVVVHCSAGIGRTGTFIVIDILIDIIREKGVDCDIDVPKTIQMVRSQ
RSGMVQTEAQYRFIYMAVQHYIETLQRRIEEEQKSKRKGHEYTNIKYSLA
DQTSGDQSPLPPCTPTPPCAEMREDSARVYENVGLMQQQKSFR

In some embodiments, acquiring knowledge that the ILC metastasis comprises an alteration in PTPN11 comprises detecting the alteration in a sample, e.g., in a sample from an ILC tumor, obtained from an individual. In some embodiments, the methods provided herein comprise detecting an alteration in PTPN11 in a sample from an individual having an ILC metastasis. In some embodiments, the methods further comprise administering an effective amount of an anti-cancer agent responsive to knowledge of the presence of an alteration in PTPN11 in a sample from an individual having an ILC metastasis. In some embodiments, the methods further comprise providing a report to a party. In some embodiments, the alteration comprises one or more of a substitution of one or more nucleotides, an insertion of one or more nucleotides, or a deletion of one or more nucleotides in PTPN11. In some embodiments, the ILC metastasis is a bone ILC metastasis, a female reproductive system ILC metastasis, a gastrointestinal ILC metastasis, a liver ILC metastasis, or a skin ILC metastasis. In some embodiments, the methods provided herein comprise acquiring knowledge that an ILC metastasis comprises an alteration in PTPN11 in a sample from an individual having a bone ILC metastasis. In some embodiments, the methods provided herein comprise acquiring knowledge that an ILC metastasis comprises an alteration in PTPN11 in a sample from an individual having a female reproductive system ILC metastasis. In some embodiments, the methods provided herein comprise acquiring knowledge that an ILC metastasis comprises an alteration in PTPN11 in a sample from an individual having a gastrointestinal ILC metastasis. In some embodiments, the methods provided herein comprise acquiring knowledge that an ILC metastasis comprises an alteration in PTPN11 in a sample from an individual having a liver ILC metastasis. In some embodiments, the methods provided herein comprise acquiring knowledge that an ILC metastasis comprises an alteration in PTPN11 in a sample from an individual having a skin ILC metastasis. In some embodiments, the methods provided herein comprise detecting an alteration in PTPN11 in a sample from an individual having a bone ILC metastasis. In some embodiments, the methods provided herein comprise detecting an alteration in PTPN11 in a sample from an individual having a female reproductive system ILC metastasis. In some embodiments, the methods provided herein comprise detecting an alteration in PTPN11 in a sample from an individual having a gastrointestinal ILC metastasis. In some embodiments, the methods provided herein comprise detecting an alteration in PTPN11 in a sample from an individual having a liver ILC metastasis. In some embodiments, the methods provided herein comprise detecting an alteration in PTPN11 in a sample from an individual having a skin ILC metastasis. In some embodiments, the alteration in PTPN11 is an activating mutation, a gain-of-function mutation, a copy number alteration (e.g., an amplification), a rearrangement, or a gene fusion. In some embodiments, the alteration in PTPN11 results in a constitutively active polypeptide or protein encoded by PTPN11. In some embodiments, the alteration in PTPN11 results in a polypeptide or protein encoded by PTPN11 that has increased activity, e.g., as compared to a polypeptide or protein encoded by a PTPN11 gene without the alteration. In some embodiments, the alteration in PTPN11 results in constitutive expression of PTPN11. In some embodiments, the alteration in PTPN11 results in overexpression of PTPN11.

In some embodiments, the methods provided herein comprise acquiring knowledge that an ILC metastasis comprises an alteration in TERT in a sample from an individual having an ILC metastasis. An exemplary nucleic acid sequence of TERT is available as Transcript ID NM_198253, provided herein as SEQ ID NO: 22, and available at the website: www[dot]ncbi[dot]nlm[dot]nih[dot]gov/nuccore/NM_198253.

An exemplary amino acid sequence of a TERT polypeptide is provided herein in SEQ ID NO: 46.

(SEQ ID NO: 46)
MPRAPRCRAVRSLLRSHYREVLPLATFVRRLGPQGWRLVQRGDPAAFRAL
VAQCLVCVPWDARPPPAAPSFRQVSCLKELVARVLQRLCERGAKNVLAFG
FALLDGARGGPPEAFTTSVRSYLPNTVTDALRGSGAWGLLLRRVGDDVLV
HLLARCALFVLVAPSCAYQVCGPPLYQLGAATQARPPPHASGPRRRLGCE
RAWNHSVREAGVPLGLPAPGARRRGGSASRSLPLPKRPRRGAAPEPERTP
VGQGSWAHPGRTRGPSDRGFCVVSPARPAEEATSLEGALSGTRHSHPSVG
RQHHAGPPSTSRPPRPWDTPCPPVYAETKHFLYSSGDKEQLRPSFLLSSL
RPSLTGARRLVETIFLGSRPWMPGTPRRLPRLPQRYWQMRPLFLELLGNH
AQCPYGVLLKTHCPLRAAVTPAAGVCAREKPQGSVAAPEEEDTDPRRLVQ
LLRQHSSPWQVYGFVRACLRRLVPPGLWGSRHNERRFLRNTKKFISLGKH
AKLSLQELTWKMSVRDCAWLRRSPGVGCVPAAEHRLREEILAKFLHWLMS
VYVVELLRSFFYVTETTFQKNRLFFYRKSVWSKLQSIGIRQHLKRVQLRE
LSEAEVRQHREARPALLTSRLRFIPKPDGLRPIVNMDYVVGARTFRREKR
AERLTSRVKALFSVLNYERARRPGLLGASVLGLDDIHRAWRTFVLRVRAQ
DPPPELYFVKVDVTGAYDTIPQDRLTEVIASIIKPQNTYCVRRYAVVQKA
AHGHVRKAFKSHVSTLTDLQPYMRQFVAHLQETSPLRDAVVIEQSSSLNE
ASSGLFDVFLRFMCHHAVRIRGKSYVQCQGIPQGSILSTLLCSLCYGDME
NKLFAGIRRDGLLLRLVDDFLLVTPHLTHAKTFLRTLVRGVPEYGCVVNL
RKTVVNFPVEDEALGGTAFVQMPAHGLFPWCGLLLDTRTLEVQSDYSSYA
RTSIRASLTFNRGFKAGRNMRRKLFGVLRLKCHSLFLDLQVNSLQTVCTN
IYKILLLQAYRFHACVLQLPFHQQVWKNPTFFLRVISDTASLCYSILKAK
NAGMSLGAKGAAGPLPSEAVQWLCHQAFLLKLTRHRVTYVPLLGSLRTAQ
TQLSRKLPGTTLTALEAAANPALPSDFKTILD

In some embodiments, acquiring knowledge that the ILC metastasis comprises an alteration in TERT comprises detecting the alteration in a sample, e.g., in a sample from an ILC tumor, obtained from an individual. In some embodiments, the methods provided herein comprise detecting an alteration in TERT in a sample from an individual having an ILC metastasis. In some embodiments, the methods further comprise administering an effective amount of an anti-cancer agent responsive to knowledge of the presence of an alteration in TERT in a sample from an individual having an ILC metastasis. In some embodiments, the methods further comprise providing a report to a party. In some embodiments, the alteration comprises one or more of a substitution of one or more nucleotides, an insertion of one or more nucleotides, or a deletion of one or more nucleotides in TERT. In some embodiments, the ILC metastasis is a bone ILC metastasis, a female reproductive system ILC metastasis, a gastrointestinal ILC metastasis, a liver ILC metastasis, or a skin ILC metastasis. In some embodiments, the methods provided herein comprise acquiring knowledge that an ILC metastasis comprises an alteration in TERT in a sample from an individual having a bone ILC metastasis. In some embodiments, the methods provided herein comprise acquiring knowledge that an ILC metastasis comprises an alteration in TERT in a sample from an individual having a female reproductive system ILC metastasis. In some embodiments, the methods provided herein comprise acquiring knowledge that an ILC metastasis comprises an alteration in TERT in a sample from an individual having a gastrointestinal ILC metastasis. In some embodiments, the methods provided herein comprise acquiring knowledge that an ILC metastasis comprises an alteration in TERT in a sample from an individual having a liver ILC metastasis. In some embodiments, the methods provided herein comprise acquiring knowledge that an ILC metastasis comprises an alteration in TERT in a sample from an individual having a skin ILC metastasis. In some embodiments, the methods provided herein comprise detecting an alteration in TERT in a sample from an individual having a bone ILC metastasis. In some embodiments, the methods provided herein comprise detecting an alteration in TERT in a sample from an individual having a female reproductive system ILC metastasis. In some embodiments, the methods provided herein comprise detecting an alteration in TERT in a sample from an individual having a gastrointestinal ILC metastasis. In some embodiments, the methods provided herein comprise detecting an alteration in TERT in a sample from an individual having a liver ILC metastasis. In some embodiments, the methods provided herein comprise detecting an alteration in TERT in a sample from an individual having a skin ILC metastasis. In some embodiments, the alteration in TERT is an activating mutation, a gain-of-function mutation, a copy number alteration (e.g., an amplification), a rearrangement, or a gene fusion. In some embodiments, the alteration in TERT results in a constitutively active polypeptide or protein encoded by TERT. In some embodiments, the alteration in TERT results in a polypeptide or protein encoded by TERT that has increased activity, e.g., as compared to a polypeptide or protein encoded by a TERT gene without the alteration. In some embodiments, the alteration in TERT results in constitutive expression of TERT. In some embodiments, the alteration in TERT results in overexpression of TERT. In some embodiments, the alteration in TERT is an alteration in the promoter of a TERT gene. In some embodiments, the alteration in the promoter of the TERT gene comprises one or more of a substitution of one or more nucleotides, an insertion of one or more nucleotides, or a deletion of one or more nucleotides. In some embodiments, the alteration in the promoter of the TERT gene comprises a −146C>T, −139_−138CC>TT, or −124C>T alteration. In some embodiments, the numbering of the nucleotides is according to the transcriptional start site according to Transcript ID NM_198253, which is provided herein as SEQ ID NO: 22.

In some embodiments, the methods provided herein comprise acquiring knowledge that an ILC metastasis comprises an alteration in ALK in a sample from an individual having an ILC metastasis. An exemplary nucleic acid sequence of ALK is available as Transcript ID NM_004304, provided herein as SEQ ID NO: 23, and available at the website: www[dot]ncbi[dot]nlm[dot]nih[dot]gov/nuccore/NM_004304.

An exemplary amino acid sequence of an ALK polypeptide is provided herein in SEQ ID NO: 47.

(SEQ ID NO: 47)
MGAIGLLWLLPLLLSTAAVGSGMGTGQRAGSPAAGPPLQPREPLSYSRLQ
RKSLAVDFVVPSLFRVYARDLLLPPSSSELKAGRPEARGSLALDCAPLLR
LLGPAPGVSWTAGSPAPAEARTLSRVLKGGSVRKLRRAKQLVLELGEEAI
LEGCVGPPGEAAVGLLQFNLSELFSWWIRQGEGRLRIRLMPEKKASEVGR
EGRLSAAIRASQPRLLFQIFGTGHSSLESPTNMPSPSPDYFTWNLTWIMK
DSFPFLSHRSRYGLECSFDFPCELEYSPPLHDLRNQSWSWRRIPSEEASQ
MDLLDGPGAERSKEMPRGSFLLLNTSADSKHTILSPWMRSSSEHCTLAVS
VHRHLQPSGRYIAQLLPHNEAAREILLMPTPGKHGWTVLQGRIGRPDNPF
RVALEYISSGNRSLSAVDFFALKNCSEGTSPGSKMALQSSFTCWNGTVLQ
LGQACDFHQDCAQGEDESQMCRKLPVGFYCNFEDGFCGWTQGTLSPHTPQ
WQVRTLKDARFQDHQDHALLLSTTDVPASESATVTSATFPAPIKSSPCEL
RMSWLIRGVLRGNVSLVLVENKTGKEQGRMVWHVAAYEGLSLWQWMVLPL
LDVSDRFWLQMVAWWGQGSRAIVAFDNISISLDCYLTISGEDKILQNTAP
KSRNLFERNPNKELKPGENSPRQTPIFDPTVHWLFTTCGASGPHGPTQAQ
CNNAYQNSNLSVEVGSEGPLKGIQIWKVPATDTYSISGYGAAGGKGGKNT
MMRSHGVSVLGIFNLEKDDMLYILVGQQGEDACPSTNQLIQKVCIGENNV
IEEEIRVNRSVHEWAGGGGGGGGATYVFKMKDGVPVPLIIAAGGGGRAYG
AKTDTFHPERLENNSSVLGLNGNSGAAGGGGGWNDNTSLLWAGKSLQEGA
TGGHSCPQAMKKWGWETRGGFGGGGGGCSSGGGGGGYIGGNAASNNDPEM
DGEDGVSFISPLGILYTPALKVMEGHGEVNIKHYLNCSHCEVDECHMDPE
SHKVICFCDHGTVLAEDGVSCIVSPTPEPHLPLSLILSVVTSALVAALVL
AFSGIMIVYRRKHQELQAMQMELQSPEYKLSKLRTSTIMTDYNPNYCFAG
KTSSISDLKEVPRKNITLIRGLGHGAFGEVYEGQVSGMPNDPSPLQVAVK
TLPEVCSEQDELDFLMEALIISKENHQNIVRCIGVSLQSLPRFILLELMA
GGDLKSFLRETRPRPSQPSSLAMLDLLHVARDIACGCQYLEENHFIHRDI
AARNCLLTCPGPGRVAKIGDFGMARDIYRASYYRKGGCAMLPVKWMPPEA
FMEGIFTSKTDTWSFGVLLWEIFSLGYMPYPSKSNQEVLEFVTSGGRMDP
PKNCPGPVYRIMTQCWQHQPEDRPNFAIILERIEYCTQDPDVINTALPIE
YGPLVEEEEKVPVRPKDPEGVPPLLVSQQAKREEERSPAAPPPLPTTSSG
KAAKKPTAAEISVRVPRGPAVEGGHVNMAFSQSNPPSELHKVHGSRNKPT
SLWNPTYGSWFTEKPTKKNNPIAKKEPHDRGNLGLEGSCTVPPNVATGRL
PGASLLLEPSSLTANMKEVPLFRLRHFPCGNVNYGYQQQGLPLEAATAPG
AGHYEDTILKSKNSMNQPGP

In some embodiments, acquiring knowledge that the ILC metastasis comprises an alteration in ALK comprises detecting the alteration in a sample, e.g., in a sample from an ILC tumor, obtained from an individual. In some embodiments, the methods provided herein comprise detecting an alteration in ALK in a sample from an individual having an ILC metastasis. In some embodiments, the methods further comprise administering an effective amount of an anti-cancer agent responsive to knowledge of the presence of an alteration in ALK in a sample from an individual having an ILC metastasis. In some embodiments, the methods further comprise providing a report to a party. In some embodiments, the alteration comprises one or more of a substitution of one or more nucleotides, an insertion of one or more nucleotides, or a deletion of one or more nucleotides in ALK. In some embodiments, the ILC metastasis is a bone ILC metastasis, a female reproductive system ILC metastasis, a gastrointestinal ILC metastasis, a liver ILC metastasis, or a skin ILC metastasis. In some embodiments, the methods provided herein comprise acquiring knowledge that an ILC metastasis comprises an alteration in ALK in a sample from an individual having a bone ILC metastasis. In some embodiments, the methods provided herein comprise acquiring knowledge that an ILC metastasis comprises an alteration in ALK in a sample from an individual having a female reproductive system ILC metastasis. In some embodiments, the methods provided herein comprise acquiring knowledge that an ILC metastasis comprises an alteration in ALK in a sample from an individual having a gastrointestinal ILC metastasis. In some embodiments, the methods provided herein comprise acquiring knowledge that an ILC metastasis comprises an alteration in ALK in a sample from an individual having a liver ILC metastasis. In some embodiments, the methods provided herein comprise acquiring knowledge that an ILC metastasis comprises an alteration in ALK in a sample from an individual having a skin ILC metastasis. In some embodiments, the methods provided herein comprise detecting an alteration in ALK in a sample from an individual having a bone ILC metastasis. In some embodiments, the methods provided herein comprise detecting an alteration in ALK in a sample from an individual having a female reproductive system ILC metastasis. In some embodiments, the methods provided herein comprise detecting an alteration in ALK in a sample from an individual having a gastrointestinal ILC metastasis. In some embodiments, the methods provided herein comprise detecting an alteration in ALK in a sample from an individual having a liver ILC metastasis. In some embodiments, the methods provided herein comprise detecting an alteration in ALK in a sample from an individual having a skin ILC metastasis. In some embodiments, the alteration in ALK is an activating mutation, a gain-of-function mutation, a copy number alteration (e.g., an amplification), a rearrangement, or a gene fusion. In some embodiments, the alteration in ALK results in a constitutively active polypeptide or protein encoded by ALK. In some embodiments, the alteration in ALK results in a polypeptide or protein encoded by ALK that has increased activity, e.g., as compared to a polypeptide or protein encoded by an ALK gene without the alteration. In some embodiments, the alteration in ALK results in constitutive expression of ALK. In some embodiments, the alteration in ALK results in overexpression of ALK.

In some embodiments, the methods provided herein comprise acquiring knowledge that an ILC metastasis comprises an alteration in KMT2D in a sample from an individual having an ILC metastasis. An exemplary nucleic acid sequence of KMT2D is available as Transcript ID NM_003482, provided herein as SEQ ID NO: 24, and available at the website: www[dot]ncbi[dot]nlm[dot]nih[dot]gov/nuccore/NM_003482.

An exemplary amino acid sequence of a KMT2D polypeptide is provided herein in SEQ ID NO: 48.

(SEQ ID NO: 48)
MDSQKLAGEDKDSEPAADGPAASEDPSATESDLPNPHVGEVSVLSSGSPR
LQETPQDCSGGPVRRCALCNCGEPSLHGQRELRRFELPFDWPRCPVVSPG
GSPGPNEAVLPSEDLSQIGFPEGLTPAHLGEPGGSCWAHHWCAAWSAGVW
GQEGPELCGVDKAIFSGISQRCSHCTRLGASIPCRSPGCPRLYHFPCATA
SGSFLSMKTLQLLCPEHSEGAAYLEEARCAVCEGPGELCDLFFCTSCGHH
YHGACLDTALTARKRAGWQCPECKVCQACRKPGNDSKMLVCETCDKGYHT
FCLKPPMEELPAHSWKCKACRVCRACGAGSAELNPNSEWFENYSLCHRCH
KAQGGQTIRSVAEQHTPVCSRFSPPEPGDTPTDEPDALYVACQGQPKGGH
VTSMQPKEPGPLQCEAKPLGKAGVQLEPQLEAPLNEEMPLLPPPEESPLS
PPPEESPTSPPPEASRLSPPPEELPASPLPEALHLSRPLEESPLSPPPEE
SPLSPPPESSPFSPLEESPLSPPEESPPSPALETPLSPPPEASPLSPPFE
ESPLSPPPEELPTSPPPEASRLSPPPEESPMSPPPEESPMSPPPEASRLF
PPFEESPLSPPPEESPLSPPPEASRLSPPPEDSPMSPPPEESPMSPPPEV
SRLSPLPVVSRLSPPPEESPLSPPPEESPTSPPPEASRLSPPPEDSPTSP
PPEDSPASPPPEDSLMSLPLEESPLLPLPEEPQLCPRSEGPHLSPRPEEP
HLSPRPEEPHLSPQAEEPHLSPQPEEPCLCAVPEEPHLSPQAEGPHLSPQ
PEELHLSPQTEEPHLSPVPEEPCLSPQPEESHLSPQSEEPCLSPRPEESH
LSPELEKPPLSPRPEKPPEEPGQCPAPEELPLFPPPGEPSLSPLLGEPAL
SEPGEPPLSPLPEELPLSPSGEPSLSPQLMPPDPLPPPLSPIITAAAPPA
LSPLGELEYPFGAKGDSDPESPLAAPILETPISPPPEANCTDPEPVPPMI
LPPSPGSPVGPASPILMEPLPPQCSPLLQHSLVPQNSPPSQCSPPALPLS
VPSPLSPIGKVVGVSDEAELHEMETEKVSEPECPALEPSATSPLPSPMGD
LSCPAPSPAPALDDFSGLGEDTAPLDGIDAPGSQPEPGQTPGSLASELKG
SPVLLDPEELAPVTPMEVYPECKQTAGQGSPCEEQEEPRAPVAPTPPTLI
KSDIVNEISNLSQGDASASFPGSEPLLGSPDPEGGGSLSMELGVSTDVSP
ARDEGSLRLCTDSLPETDDSLLCDAGTAISGGKAEGEKGRRRSSPARSRI
KQGRSSSFPGRRRPRGGAHGGRGRGRARLKSTASSIETLVVADIDSSPSK
EEEEEDDDTMQNTVVLFSNTDKFVLMQDMCVVCGSFGRGAEGHLLACSQC
SQCYHPYCVNSKITKVMLLKGWRCVECIVCEVCGQASDPSRLLLCDDCDI
SYHTYCLDPPLLTVPKGGWKCKWCVSCMQCGAASPGFHCEWQNSYTHCGP
CASLVTCPICHAPYVEEDLLIQCRHCERWMHAGCESLFTEDDVEQAADEG
FDCVSCQPYVVKPVAPVAPPELVPMKVKEPEPQYFRFEGVWLTETGMALL
RNLTMSPLHKRRQRRGRLGLPGEAGLEGSEPSDALGPDDKKDGDLDTDEL
LKGEGGVEHMECEIKLEGPVSPDVEPGKEETEESKKRKRKPYRPGIGGEM
VRQRKSHTRTKKGPAAQAEVLSGDGQPDEVIPADLPAEGAVEQSLAEGDE
KKKQQRRGRKKSKLEDMFPAYLQEAFFGKELLDLSRKALFAVGVGRPSFG
LGTPKAKGDGGSERKELPTSQKGDDGPDIADEESRGLEGKADTPGPEDGG
VKASPVPSDPEKPGTPGEGMLSSDLDRISTEELPKMESKDLQQLFKDVLG
SEREQHLGCGTPGLEGSRTPLQRPFLQGGLPLGNLPSSSPMDSYPGLCQS
PFLDSRERGGFFSPEPGEPDSPWTGSGGTTPSTPTTPTTEGEGDGLSYNQ
RSLQRWEKDEELGQLSTISPVLYANINFPNLKQDYPDWSSRCKQIMKLWR
KVPAADKAPYLQKAKDNRAAHRINKVQKQAESQINKQTKVGDIARKTDRP
ALHLRIPPQPGALGSPPPAAAPTIFIGSPTTPAGLSTSADGFLKPPAGSV
PGPDSPGELFLKLPPQVPAQVPSQDPFGLAPAYPLEPRFPTAPPTYPPYP
SPTGAPAQPPMLGASSRPGAGQPGEFHTTPPGTPRHQPSTPDPFLKPRCP
SLDNLAVPESPGVGGGKASEPLLSPPPFGESRKALEVKKEELGASSPSYG
PPNLGFVDSPSSGTHLGGLELKTPDVFKAPLTPRASQVEPQSPGLGLRPQ
EPPPAQALAPSPPSHPDIFRPGSYTDPYAQPPLTPRPQPPPPESCCALPP
RSLPSDPFSRVPASPQSQSSSQSPLTPRPLSAEAFCPSPVTPRFQSPDPY
SRPPSRPQSRDPFAPLHKPPRPQPPEVAFKAGSLAHTSLGAGGFPAALPA
GPAGELHAKVPSGQPPNFVRSPGTGAFVGTPSPMRFTFPQAVGEPSLKPP
VPQPGLPPPHGINSHFGPGPTLGKPQSTNYTVATGNFHPSGSPLGPSSGS
TGESYGLSPLRPPSVLPPPAPDGSLPYLSHGASQRSGITSPVEKREDPGT
GMGSSLATAELPGTQDPGMSGLSQTELEKQRQRQRLRELLIRQQIQRNTL
RQEKETAAAAAGAVGPPGSWGAEPSSPAFEQLSRGQTPFAGTQDKSSLVG
LPPSKLSGPILGPGSFPSDDRLSRPPPPATPSSMDVNSRQLVGGSQAFYQ
RAPYPGSLPLQQQQQQLWQQQQATAATSMRFAMSARFPSTPGPELGRQAL
GSPLAGISTRLPGPGEPVPGPAGPAQFIELRHNVQKGLGPGGTPFPGQGP
PQRPRFYPVSEDPHRLAPEGLRGLAVSGLPPQKPSAPPAPELNNSLHPTP
HTKGPTLPTGLELVNRPPSSTELGRPNPLALEAGKLPCEDPELDDDFDAH
KALEDDEELAHLGLGVDVAKGDDELGTLENLETNDPHLDDLLNGDEFDLL
AYTDPELDTGDKKDIFNEHLRLVESANEKAEREALLRGVEPGPLGPEERP
PPAADASEPRLASVLPEVKPKVEEGGRHPSPCQFTIATPKVEPAPAANSL
GLGLKPGQSMMGSRDTRMGTGPFSSSGHTAEKASFGATGGPPAHLLTPSP
LSGPGGSSLLEKFELESGALTLPGGPAASGDELDKMESSLVASELPLLIE
DLLEHEKKELQKKQQLSAQLQPAQQQQQQQQQHSLLSAPGPAQAMSLPHE
GSSPSLAGSQQQLSLGLAGARQPGLPQPLMPTQPPAHALQQRLAPSMAMV
SNQGHMLSGQHGGQAGLVPQQSSQPVLSQKPMGTMPPSMCMKPQQLAMQQ
QLANSFFPDTDLDKFAAEDIIDPIAKAKMVALKGIKKVMAQGSIGVAPGM
NRQQVSLLAQRLSGGPSSDLQNHVAAGSGQERSAGDPSQPRPNPPTFAQG
VINEADQRQYEEWLFHTQQLLQMQLKVLEEQIGVHRKSRKALCAKQRTAK
KAGREFPEADAEKLKLVTEQQSKIQKQLDQVRKQQKEHTNLMAEYRNKQQ
QQQQQQQQQQQQHSAVLALSPSQSPRLLTKLPGQLLPGHGLQPPQGPPGG
QAGGLRLTPGGMALPGQPGGPFLNTALAQQQQQQHSGGAGSLAGPSGGFF
PGNLALRSLGPDSRLLQERQLQLQQQRMQLAQKLQQQQQQQQQQQHLLGQ
VAIQQQQQQGPGVQTNQALGPKPQGLMPPSSHQGLLVQQLSPQPPQGPQG
MLGPAQVAVLQQQHPGALGPQGPHRQVLMTQSRVLSSPQLAQQGQGLMGH
RLVTAQQQQQQQQHQQQGSMAGLSHLQQSLMSHSGQPKLSAQPMGSLQQL
QQQQQLQQQQQLQQQQQQQLQQQQQLQQQQLQQQQQQQQLQQQQQQQLQQ
QQQQLQQQQQQQQQQFQQQQQQQQMGLLNQSRTLLSPQQQQQQQVALGPG
MPAKPLQHFSSPGALGPTLLLTGKEQNTVDPAVSSEATEGPSTHQGGPLA
IGTTPESMATEPGEVKPSLSGDSQLLLVQPQPQPQPSSLQLQPPLRLPGQ
QQQQVSLLHTAGGGSHGQLGSGSSSEASSVPHLLAQPSVSLGDQPGSMTQ
NLLGPQQPMLERPMQNNTGPQPPKPGPVLQSGQGLPGVGIMPTVGQLRAQ
LQGVLAKNPQLRHLSPQQQQQLQALLMQRQLQQSQAVRQTPPYQEPGTQT
SPLQGLLGCQPQLGGFPGPQTGPLQELGAGPRPQGPPRLPAPPGALSTGP
VLGPVHPTPPPSSPQEPKRPSQLPSPSSQLPTEAQLPPTHPGTPKPQGPT
LEPPPGRVSPAAAQLADTLFSKGLGPWDPPDNLAETQKPEQSSLVPGHLD
QVNGQVVPEASQLSIKQEPREEPCALGAQSVKREANGEPIGAPGTSNHLL
LAGPRSEAGHLLLQKLLRAKNVQLSTGRGSEGLRAEINGHIDSKLAGLEQ
KLQGTPSNKEDAAARKPLTPKPKRVQKASDRLVSSRKKLRKEDGVRASEA
LLKQLKQELSLLPLTEPAITANFSLFAPFGSGCPVNGQSQLRGAFGSGAL
PTGPDYYSQLLTKNNLSNPPTPPSSLPPTPPPSVQQKMVNGVTPSEELGE
HPKDAASARDSERALRDTSEVKSLDLLAALPTPPHNQTEDVRMESDEDSD
SPDSIVPASSPESILGEEAPRFPHLGSGRWEQEDRALSPVIPLIPRASIP
VFPDTKPYGALGLEVPGKLPVTTWEKGKGSEVSVMLTVSAAAAKNLNGVM
VAVAELLSMKIPNSYEVLFPESPARAGTEPKKGEAEGPGGKEKGLEGKSP
DTGPDWLKQFDAVLPGYTLKSQLDILSLLKQESPAPEPPTQHSYTYNVSN
LDVRQLSAPPPEEPSPPPSPLAPSPASPPTEPLVELPTEPLAEPPVPSPL
PLASSPESARPKPRARPPEEGEDSRPPRLKKWKGVRWKRLRLLLTIQKGS
GRQEDEREVAEFMEQLGTALRPDKVPRDMRRCCFCHEEGDGATDGPARLL
NLDLDLWVHLNCALWSTEVYETQGGALMNVEVALHRGLLTKCSLCQRTGA
TSSCNRMRCPNVYHFACAIRAKCMFFKDKTMLCPMHKIKGPCEQELSSFA
VERRVYIERDEVKQIASIIQRGERLHMFRVGGLVFHAIGQLLPHQMADFH
SATALYPVGYEATRIYWSLRTNNRRCCYRCSIGENNGRPEFVIKVIEQGL
EDLVFTDASPQAVWNRIIEPVAAMRKEADMLRLFPEYLKGEELFGLTVHA
VLRIAESLPGVESCQNYLFRYGRHPLMELPLMINPTGCARSEPKILTHYK
RPHTLNSTSMSKAYQSTFTGETNTPYSKQFVHSKSSQYRRLRTEWKNNVY
LARSRIQGLGLYAAKDLEKHTMVIEYIGTIIRNEVANRREKIYEEQNRGI
YMFRINNEHVIDATLTGGPARYINHSCAPNCVAEVVTFDKEDKIIIISSR
RIPKGEELTYDYQFDFEDDQHKIPCHCGAWNCRKWMN

In some embodiments, acquiring knowledge that the ILC metastasis comprises an alteration in KMT2D comprises detecting the alteration in a sample, e.g., in a sample from an ILC tumor, obtained from an individual. In some embodiments, the methods provided herein comprise detecting an alteration in KMT2D in a sample from an individual having an ILC metastasis. In some embodiments, the methods further comprise administering an effective amount of an anti-cancer agent responsive to knowledge of the presence of an alteration in KMT2D in a sample from an individual having an ILC metastasis. In some embodiments, the alteration in KMT2D comprises a KMT2D rearrangement. In some embodiments, the methods further comprise providing a report to a party. In some embodiments, the ILC metastasis is a bone ILC metastasis, a female reproductive system ILC metastasis, a gastrointestinal ILC metastasis, a liver ILC metastasis, or a skin ILC metastasis. In some embodiments, the methods provided herein comprise acquiring knowledge that an ILC metastasis comprises an alteration in KMT2D in a sample from an individual having a bone ILC metastasis. In some embodiments, the methods provided herein comprise acquiring knowledge that an ILC metastasis comprises an alteration in KMT2D in a sample from an individual having a female reproductive system ILC metastasis. In some embodiments, the methods provided herein comprise acquiring knowledge that an ILC metastasis comprises an alteration in KMT2D in a sample from an individual having a gastrointestinal ILC metastasis. In some embodiments, the methods provided herein comprise acquiring knowledge that an ILC metastasis comprises an alteration in KMT2D in a sample from an individual having a liver ILC metastasis. In some embodiments, the methods provided herein comprise acquiring knowledge that an ILC metastasis comprises an alteration in KMT2D in a sample from an individual having a skin ILC metastasis. In some embodiments, the methods provided herein comprise detecting an alteration in KMT2D in a sample from an individual having a bone ILC metastasis. In some embodiments, the methods provided herein comprise detecting an alteration in KMT2D in a sample from an individual having a female reproductive system ILC metastasis. In some embodiments, the methods provided herein comprise detecting an alteration in KMT2D in a sample from an individual having a gastrointestinal ILC metastasis. In some embodiments, the methods provided herein comprise detecting an alteration in KMT2D in a sample from an individual having a liver ILC metastasis. In some embodiments, the methods provided herein comprise detecting an alteration in KMT2D in a sample from an individual having a skin ILC metastasis. In some embodiments, the alteration in KMT2D is an inactivating mutation, a loss-of-function mutation, a copy number alteration (e.g., a deletion), a rearrangement, or a gene fusion. In some embodiments, the alteration in KMT2D results in an inactive polypeptide or protein encoded by KMT2D. In some embodiments, the alteration in KMT2D results in a polypeptide or protein encoded by KMT2D that has reduced activity, e.g., as compared to a polypeptide or protein encoded by a KMT2D gene without the alteration. In some embodiments, the alteration in KMT2D results in reduced expression of KMT2D. In some embodiments, the alteration in KMT2D results in loss of expression of KMT2D.

Detection of Gene Alterations in Nucleic Acids

In some embodiments, the methods provided herein comprise acquiring knowledge of an alteration in one or more genes in a sample from an individual. In some embodiments, the methods provided herein comprise detecting an alteration in one or more genes in a sample from an individual. In some embodiments, the one or more genes include PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D. In some embodiments, the acquiring knowledge comprises detecting the alteration in a sample obtained from an individual. In some embodiments, an alteration in a gene is detected in a nucleic acid encoding the gene, e.g., genomic DNA or fragments thereof, a cDNA or fragments thereof, or an RNA (e.g., an mRNA) or fragments thereof.

In some embodiments, the methods comprise acquiring knowledge of or detecting an alteration in PIK3CA, e.g., in a nucleic acid encoding PIK3CA, e.g., genomic DNA or fragments thereof, a cDNA or fragments thereof, or an RNA (e.g., an mRNA) or fragments thereof. In some embodiments, the alteration in PIK3CA comprises an alteration that results in one or more amino acid substitutions in a polypeptide encoded by the PIK3CA gene. In some embodiments, the one or more amino acid substitutions are at amino acid position E81, R108, K111, G118, N345, D350, E365, E418, C420, E453, P539, E542, E545, Q546, E726, E970, M1004, M1043, N1044, H1047, G1049, or any combination thereof, wherein the numbering of the residues is according to SEQ ID NO: 26 or according to the amino acid sequence of a human PIK3CA protein. In some embodiments, the one or more amino acid substitutions in a polypeptide encoded by the PIK3CA gene comprise a E81K, R108H, K111N, G118D, N345K, D350N, E365K, E418K, C420R, E453K, E453Q, P539R, E542K, E545K, E545A, E545Q, Q546R, Q546K, E726K, E970K, M1004I, M1043I, N1044K, H1047R, H1047L, or G1049R amino acid substitution, or any combination thereof, wherein the numbering of the residues is according to SEQ ID NO: 26 or according to the amino acid sequence of a human PIK3CA protein. In some embodiments, the alteration in PIK3CA comprises an alteration that results in a deletion of one or more amino acids in a polypeptide encoded by the PIK3CA gene. In some embodiments, the deletion of one or more amino acids in a polypeptide encoded by the PIK3CA gene comprises a deletion of amino acid residue E110 (E110del), wherein the numbering of the residues is according to SEQ ID NO: 26, or according to the amino acid sequence of a human PIK3CA protein.

In some embodiments, the methods comprise acquiring knowledge of or detecting an alteration in ESR1, e.g., in a nucleic acid encoding ESR1, e.g., genomic DNA or fragments thereof, a cDNA or fragments thereof, or an RNA (e.g., an mRNA) or fragments thereof. In some embodiments, the alteration in ESR1 is an alteration resulting in one or more amino acid substitutions in a polypeptide encoded by the ESR1 gene. In some embodiments, the one or more amino acid substitutions are at amino acid position E380, V418, S463, V533, L536, Y537, D538, or any combination thereof, wherein the numbering of the residues is according to SEQ ID NO: 29, or according to the amino acid sequence of a human ESR1 protein. In some embodiments, the one or more amino acid substitutions in a polypeptide encoded by the ESR1 gene comprise a E380Q, V418E, S463P, V533M, L536Q, L536H, L536P, L536R, Y537S, Y537N, Y537C, Y537D, or D538G amino acid substitution, or any combination thereof, wherein the numbering of the residues is according to SEQ ID NO: 29, or according to the amino acid sequence of a human ESR1 protein. In some embodiments, the alteration in ESR1 is an alteration resulting in a deletion of one or more amino acids in a polypeptide encoded by the ESR1 gene. In some embodiments, the deletion of one or more amino acids in the polypeptide encoded by the ESR1 gene is a deletion of amino acid V422 (V422del) and/or a deletion of amino acids V533-L536 (V533_L536del) in a polypeptide encoded by the ESR1 gene, wherein the numbering of the residues is according to SEQ ID NO: 29, or according to the amino acid sequence of a human ESR1 protein.

In some embodiments, the methods comprise acquiring knowledge of or detecting an alteration in KRAS, e.g., in a nucleic acid encoding KRAS, e.g., genomic DNA or fragments thereof, a cDNA or fragments thereof, or an RNA (e.g., an mRNA) or fragments thereof. In some embodiments, the alteration in KRAS results in one or more amino acid substitutions in a polypeptide encoded by the KRAS gene. In some embodiments, the one or more amino acid substitutions are at amino acid position G12, G13, L19, Q61, A146, K147, F156, or any combination thereof, wherein the numbering of the residues is according to SEQ ID NO: 33, or according to the amino acid sequence of a human KRAS protein. In some embodiments, the one or more amino acid substitutions in a polypeptide encoded by the KRAS gene comprise a G12V, G12D, G12A, G12R, G12S, G12C, G12L, G13D, L19F, Q61H, Q61K, Q61E, A146T, K147N, or F156L amino acid substitution, or any combination thereof, wherein the numbering of the residues is according to SEQ ID NO: 33, or according to the amino acid sequence of a human KRAS protein.

In some embodiments, the methods comprise acquiring knowledge of or detecting an alteration in ERBB2, e.g., in a nucleic acid encoding ERBB2, e.g., genomic DNA or fragments thereof, a cDNA or fragments thereof, or an RNA (e.g., an mRNA) or fragments thereof. In some embodiments, the alteration in ERBB2 results in one or more amino acid substitutions in a polypeptide encoded by the ERBB2 gene. In some embodiments, the one or more amino acid substitutions are at amino acid position S310, S653, V659, R678, V697, E717, T733, L755, I767, D769, G776, V777, T798, V842, L869, or any combination thereof, wherein the numbering of the residues is according to SEQ ID NO: 34, or according to the amino acid sequence of a human ERBB2 protein. In some embodiments, the one or more amino acid substitutions in a polypeptide encoded by the ERBB2 gene comprise a S310F, S310Y, S653C, V659D, R678Q, V697L, E717K, T733I, L755S, L755P, I767M, D769N, D769Y, D769H, G776V, V777L, T798I, V842I, or L869R amino acid substitution, or any combination thereof, wherein the numbering of the residues is according to SEQ ID NO: 34, or according to the amino acid sequence of a human ERBB2 protein. In some embodiments, the alteration in ERBB2 results in a deletion of one or more amino acid residues in a polypeptide encoded by the ERBB2 gene. In some embodiments, the deletion of one or more amino acid residues in a polypeptide encoded by the ERBB2 gene is a deletion of amino acids L755-T759 (L755_T759del) and/or amino acids L755-E757 (L755_E757del) in a polypeptide encoded by the ERBB2 gene, wherein the numbering of the residues is according to SEQ ID NO: 34, or according to the amino acid sequence of a human ERBB2 protein. In some embodiments, the alteration in ERBB2 results in an insertion of one or more amino acid residues in a polypeptide encoded by the ERBB2 gene. In some embodiments, the insertion of one or more amino acid residues in a polypeptide encoded by the ERBB2 gene is an insertion of the amino acid sequence YVMA (SEQ ID NO: 51) between amino acid residues A775 and G776 (A775_G776insYVMA), and/or of the amino acid sequence GSP (SEQ ID NO: 52) between amino acid residues P780 and Y781 (P780_Y781insGSP) in a polypeptide encoded by the ERBB2 gene, wherein the numbering of the residues is according to SEQ ID NO: 34, or according to the amino acid sequence of a human ERBB2 protein.

In some embodiments, the methods comprise acquiring knowledge of or detecting an alteration in FGFR2, e.g., in a nucleic acid encoding FGFR2, e.g., genomic DNA or fragments thereof, a cDNA or fragments thereof, or an RNA (e.g., an mRNA) or fragments thereof. In some embodiments, the alteration in FGFR2 results in one or more amino acid substitutions in a polypeptide encoded by the FGFR2 gene. In some embodiments, the one or more amino acid substitutions are at amino acid position S252, P253, Y375, C382, M391, V395, M537, N549, K659, R664, or any combination thereof, wherein the numbering of the residues is according to SEQ ID NO: 38, or according to the amino acid sequence of a human FGFR2 protein. In some embodiments, the one or more amino acid substitutions in a polypeptide encoded by the FGFR2 gene comprise a S252W, P253R, Y375C, C382R, M391R, V395D, M537I, N549D, N549K, K659M, K659E, K659N, or R664W amino acid substitution, or any combination thereof, wherein the numbering of the residues is according to SEQ ID NO: 38, or according to the amino acid sequence of a human FGFR2 protein. In some embodiments, the alteration results in a frameshift in FGFR2 (e.g., in an mRNA sequence encoded by the FGFR2 gene and in the resulting amino acid sequence). In some embodiments, the frameshift is an S799fs*22 frameshift, wherein the numbering of the residues is according to SEQ ID NO: 38, or according to the amino acid sequence of a human FGFR2 protein.

In some embodiments, the methods comprise acquiring knowledge of or detecting an alteration in BRAF, e.g., in a nucleic acid encoding BRAF, e.g., genomic DNA or fragments thereof, a cDNA or fragments thereof, or an RNA (e.g., an mRNA) or fragments thereof. In some embodiments, the alteration in BRAF results in one or more amino acid substitutions in a polypeptide encoded by the BRAF gene. In some embodiments, the one or more amino acid substitutions are at amino acid position D380, G464, G466, S467, G469, L485, L584, E586, D594, V600, or any combination thereof, wherein the numbering of the residues is according to SEQ ID NO: 35 or according to the amino acid sequence of a human BRAF protein. In some embodiments, the one or more amino acid substitutions in a polypeptide encoded by the BRAF gene comprise a D380H, G464R, G466E, S467L, G469A, G469E, G469R, L485F, L584F, E586K, D594N, D594G, or V600E amino acid substitution, or any combination thereof, wherein the numbering of the residues is according to SEQ ID NO: 35, or according to the amino acid sequence of a human BRAF protein.

In some embodiments, the methods comprise acquiring knowledge of or detecting an alteration in TERT, e.g., in a nucleic acid encoding TERT, e.g., genomic DNA or fragments thereof, a cDNA or fragments thereof, or an RNA (e.g., an mRNA) or fragments thereof. In some embodiments, the alteration in TERT is an alteration in the promoter of a TERT gene. In some embodiments, the alteration in the promoter of the TERT gene comprises one or more of a substitution of one or more nucleotides, an insertion of one or more nucleotides, or a deletion of one or more nucleotides. In some embodiments, the alteration in the promoter of the TERT gene comprises a −146C>T, −139_−138CC>TT, or −124C>T alteration, or any combination thereof.

An alteration in one or more genes may be detected using any suitable method known in the art, such as a nucleic acid hybridization assay, an amplification-based assay (e.g., polymerase chain reaction, PCR), a PCR-RFLP assay, real-time PCR, sequencing (e.g., Sanger sequencing or next-generation sequencing), a screening analysis (e.g., using karyotype methods), fluorescence in situ hybridization (FISH), break away FISH, spectral karyotyping, multiplex-FISH, comparative genomic hybridization, in situ hybridization, single specific primer-polymerase chain reaction (SSP-PCR), HPLC, or mass-spectrometric genotyping. Methods of analyzing samples, e.g., to detect an alteration in one or more genes, are described in U.S. Pat. No. 9,340,830 and in WO2012092426A1, which are hereby incorporated by reference in their entirety.

Sequencing

In some embodiments, a gene alteration, e.g., a gene alteration in one or more of PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D is detected using a sequencing method. Any method of sequencing known in the art can be used to detect a gene alteration, e.g., a gene alteration in one or more of PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D. Exemplary sequencing methods that may be used include those based on techniques developed by Maxam and Gilbert or Sanger. Automated sequencing procedures may also be used, e.g., including sequencing by mass spectrometry.

In some embodiments, a gene alteration, e.g., a gene alteration in one or more of PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D is detected using next-generation sequencing (NGS). Next-generation sequencing includes any sequencing method that determines the nucleotide sequence of either individual nucleic acid molecules or clonally expanded proxies for individual nucleic acid molecules in a highly parallel fashion (e.g., greater than 10⁵ molecules may be sequenced simultaneously). Next generation sequencing methods suitable for use according to the methods provided herein are known in the art and include, without limitation, massively parallel short-read sequencing, template-based sequencing, pyrosequencing, real-time sequencing comprising imaging the continuous incorporation of dye-labeling nucleotides during DNA synthesis, nanopore sequencing, sequencing by hybridization, nano-transistor array based sequencing, polony sequencing, scanning tunneling microscopy (STM)-based sequencing, or nanowire-molecule sensor-based sequencing. See, e.g., Metzker, M. (2010) Nature Biotechnology Reviews 11:31-46, which is hereby incorporated by reference. Exemplary NGS methods and platforms that may be used include, without limitation, the HeliScope Gene Sequencing system from Helicos BioSciences (Cambridge, MA., USA), the PacBio RS system from Pacific Biosciences (Menlo Park, CA, USA), massively parallel short-read sequencing such as the Solexa sequencer and other methods and platforms from Illumina Inc. (San Diego, CA, USA), 454 sequencing from 454 LifeSciences (Branford, CT, USA), Ion Torrent sequencing from ThermoFisher (Waltham, MA, USA), or the SOLiD sequencer from Applied Biosystems (Foster City, CA, USA). Additional exemplary methods and platforms that may be used include, without limitation, the Genome Sequencer (GS) FLX System from Roche (Basel, CHE), the G.007 polonator system, the Solexa Genome Analyzer, HiSeq 2500, HiSeq3000, HiSeq 4000, and NovaSeq 6000 platforms from Illumina Inc. (San Diego, CA, USA). In some embodiments, one or more gene alterations of the disclosure are detected using hybrid capture-based sequencing (hybrid capture-based next-generation sequencing (NGS)), e.g., using adaptor ligation-based libraries. See, e.g., Frampton, G. M. et al. (2013) Nat. Biotech. 31:1023-1031.

In some embodiments of any of the methods provided herein, the methods comprise providing a sample from an individual, wherein the sample comprises one or more nucleic acids. In some embodiments, the methods further comprise preparing a nucleic acid sequencing library from the one or more nucleic acids in the sample. Methods for the preparation of nucleic acid sequencing libraries, e.g., suitable for any of the sequencing methods described herein (e.g., NGS and/or hybrid-capture NGS), are known in the art. In some embodiments, the sequencing library is prepared as described in Frampton et al., (2013) Nat Biotechnol, 31:1023-1031. In an exemplary method, nucleic acids, e.g., double stranded DNA (dsDNA), are fragmented, for example, using sonication. In some embodiments, nucleic acids are fragmented to a length of about 200 base pairs. In some embodiments, the fragmented nucleic acids are purified, e.g., using any suitable method, such as using AMPure XP Beads (Agencourt) and/or solid phase reversible immobilization (SPRI) methods. In some embodiments, sequencing library construction using the purified nucleic acids is carried out using any suitable method, e.g., using commercially available library preparation kits, such as an NEBNext kit (e.g., available from New England Biolabs). In some embodiments, library preparation is performed using a “with-bead” protocol. See, e.g., Fisher et al., Genome Biol (2011) 12:R1. In some embodiments, the library preparation method is selected based on the sequencing method used, e.g., an NEBNext kit is suitable for use with NGS sequencing platforms from Illumina Inc. In some embodiments, a sequencing library indexed, e.g., with barcodes such as six base pair barcodes, is amplified, e.g., using any suitable method, such as PCR. In some embodiments, amplified nucleic acids are purified using any suitable method, such as SPRI purification. In some embodiments, the methods further comprise quantifying the amplified and/or purified nucleic acids, e.g., by qPCR. In some embodiments, the methods further comprise sizing the amplified and/or purified nucleic acids using any suitable method, such as using a LabChip GX system, e.g., available from Caliper Life Sciences. In some embodiments, size selection is not performed.

In some embodiments, the methods further comprise selectively enriching for one or more nucleic acids (e.g., one or more nucleic acids comprising one or more gene alterations described herein) to produce an enriched sample. In some embodiments, the selectively enriching is performed on a sequencing library, e.g., a sequencing library prepared according to the methods described herein. In some embodiments, the selectively enriching is performed as described in Frampton et al., (2013) Nat Biotechnol, 31:1023-1031. In an exemplary process, the methods comprise performing solution hybridization using 5′-biotinylated DNA oligonucleotide baits, which may be prepared or synthesized using any suitable method known in the art, e.g., as described in Frampton et al., (2013) Nat Biotechnol, 31:1023-1031. In some embodiments, the methods comprise denaturing the sequencing library. In some embodiments, denaturing is performed at a temperature of about 95° C., e.g., for about 5 minutes. In some embodiments, the methods further comprise incubating the denatured sequencing library at a temperature of about 68° C., e.g., for about 5 minutes. In some embodiments, the methods further comprise mixing the sequencing library with baits, and optionally Cot, salmon sperm, and/or adaptor-specific blocker DNA in hybridization buffer. In some embodiments, the mixture is incubated for about 24 hours. In some embodiments, the methods further comprise capturing sequencing library-bait duplexes using any suitable method, such as using paramagnetic MyOne streptavidin beads (available from Invitrogen). In some embodiments, the methods further comprise washing to remove off-target library. In some embodiments, the methods further comprise amplifying the captured sequencing library, e.g., using PCR. In some embodiments, the methods further comprise purifying the amplification products using any suitable method, such as SPRI purification. In some embodiments, the methods further comprise quantifying the amplified and/or purified nucleic acids, e.g., by qPCR. In some embodiments, the methods further comprise sizing the amplified and/or purified nucleic acids using any suitable method, such as using a LabChip GX system, e.g., available from Caliper Life Sciences. In some embodiments, the methods further comprise sequencing using any suitable method or system known in the art, e.g., as described herein. In some embodiments, sequencing is performed using an Illumina HiSeq 2000 system. In some embodiments, sequencing is performed using paired-end sequencing. In some embodiments, the sequencing is performed as described in Frampton et al., (2013) Nat Biotechnol, 31:1023-1031.

In some embodiments, the methods further comprise analyzing sequence data obtained from the sequencing, e.g., a plurality of sequence reads, for the presence or absence of one or more gene alterations of the disclosure. In some embodiments, the analysis is performed as described in Frampton et al., (2013) Nat Biotechnol, 31:1023-1031. In some embodiments, the methods comprise aligning the sequence data to the human genome using any suitable method, such as using a BWA aligner (e.g., v0.5.9). See, e.g., Li and Durbin, Bioinformatics (2010) 26:589-595. In some embodiments, the methods comprise aligning the sequence data to human genome version hg19. In some embodiments, PCR duplicate reads are removed, and/or sequence data metrics are collected using any suitable method, such as using Picard 1.47 (see, e.g., picard.sourceforge.net and/or broadinstitute.github.io/picard/) and/or Samtools 0.1.12a (see, e.g., Li et al., Bioinformatics (2009) 25:2078-2079). In some embodiments, the methods further comprise performing local alignment optimization using any suitable method, e.g., using GATK 1.0.4705 (see, e.g., DePristo et al., Nat Genet (2011) 43:491-498).

In some embodiments, the presence or absence of one or more gene alterations of the disclosure is detected using any suitable method known in the art, e.g., as described in Frampton et al., (2013) Nat Biotechnol, 31:1023-1031. In some embodiments, base substitution alterations are detected using Bayesian methodology, which allows detection of novel somatic mutations at low mutant allele frequency (MAF) and increased sensitivity for mutations at hotspot sites through the incorporation of tissue-specific prior expectations. See, e.g., Kim et al., Cancer Discov (2011) 1:44-53 and Frampton et al., (2013) Nat Biotechnol, 31:1023-1031. In some embodiments, insertion/deletion (indel) alterations are detected using any suitable method, such as de novo local assembly, e.g., using the de Bruijn approach, see, e.g., Compeau et al., Nat Biotechnol (2011) 29:987-991 and Frampton et al., (2013) Nat Biotechnol, 31:1023-1031. In some embodiments, copy number alterations are detected using any suitable method, such as using a comparative genomic hybridization (CGH)-like method, see, e.g., Frampton et al., (2013) Nat Biotechnol, 31:1023-1031. In some embodiments, gene fusion and genomic rearrangement alterations are detected using any suitable method, such as by analyzing chimeric read pairs (read pairs for which reads map to separate chromosomes, or at a distance of over 10 Mbp), see, e.g., Frampton et al., (2013) Nat Biotechnol, 31:1023-1031. In some embodiments, rearrangements are annotated for predicted function (e.g., creation of fusion gene or tumor suppressor inactivation).

Array-Based Methods

In some embodiments, a gene alteration, e.g., a gene alteration in one or more of PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D is detected using an array-based method, such as array-based comparative genomic hybridization (CGH) methods. In array-based CGH methods, a first sample of nucleic acids (e.g., from a sample, such as from a tumor) is labeled with a first label, while a second sample of nucleic acids (e.g., a control, such as from a healthy cell/tissue) is labeled with a second label. In some embodiments, equal quantities of the two samples are mixed and co-hybridized to a DNA microarray of several thousand evenly-spaced cloned DNA fragments or oligonucleotides, which have been spotted in triplicate on the array. After hybridization, digital imaging systems are used to capture and quantify the relative fluorescence intensities of each of the hybridized fluorophores. The resulting ratio of the fluorescence intensities is proportional to the ratio of the copy numbers of DNA sequences in the two samples. In some embodiments, where there are gene alterations, e.g., chromosomal deletions or multiplications, differences in the ratio of the signals from the two labels are detected and the ratio provides a measure of the copy number. Array-based CGH can also be performed with single-color labeling. In single color CGH, a control (e.g., control nucleic acid sample, such as from a healthy cell/tissue) is labeled and hybridized to one array and absolute signals are read, and a test sample (e.g., a nucleic acid sample obtained from an individual or from a tumor) is labeled and hybridized to a second array (with identical content) and absolute signals are read. Copy number differences are calculated based on absolute signals from the two arrays.

Amplification-Based Methods

In some embodiments, a gene alteration, e.g., a gene alteration in one or more of PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D is detected using an amplification-based method. As is known in the art, in such amplification-based methods, a sample of nucleic acids, such as a sample obtained from an individual or from a tumor, is used as a template in an amplification reaction (e.g., Polymerase Chain Reaction (PCR)) using one or more oligonucleotides or primers. The presence of a gene alteration, e.g., a gene alteration in one or more of PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D, can be determined based on the presence or absence of an amplification product. Quantitative amplification methods are also known in the art and may be used according to the methods provided herein. Methods of measurement of DNA copy number at microsatellite loci using quantitative PCR analysis are known in the art. The known nucleotide sequence for genes, e.g., PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D, is sufficient to enable one of skill in the art to routinely select primers to amplify any portion of the gene. Fluorogenic quantitative PCR can also be used. In fluorogenic quantitative PCR, quantitation is based on the amount of fluorescence signals, e.g., TaqMan and Sybr green.

Other amplification methods suitable for use according to the methods provided herein include, e.g., ligase chain reaction (LCR), transcription amplification, self-sustained sequence replication, dot PCR, and linker adapter PCR.

In Situ Hybridization Methods

In some embodiments, a gene alteration, e.g., a gene alteration in one or more of PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D is detected using an in situ hybridization method, such as a fluorescence in situ hybridization (FISH) method.

In some embodiments, FISH analysis is used to identify a chromosomal rearrangement resulting in a gene alteration in one or more genes, e.g., PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D. In some embodiments, FISH analysis is used to identify an RNA molecule comprising a gene alteration in one or more genes, e.g., PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D. Methods for performing FISH are known in the art and can be used in nearly any type of tissue. In FISH analysis, nucleic acid probes which are detectably labeled, e.g. fluorescently labeled, are allowed to bind to specific regions of DNA, e.g., a chromosome, or an RNA, e.g., an mRNA, and then examined, e.g., through a microscope. See, for example, U.S. Pat. No. 5,776,688. DNA or RNA molecules are first fixed onto a slide, the labeled probe is then hybridized to the DNA or RNA molecules, and then visualization is achieved, e.g., using enzyme-linked label-based detection methods known in the art. Generally, the resolution of FISH analysis is on the order of detection of 60 to 100000 nucleotides, e.g., 60 base pairs (bp) up to 100 kilobase pairs of DNA. Nucleic acid probes used in FISH analysis comprise single stranded nucleic acids. Such probes are typically at least about 50 nucleotides in length. In some embodiments, probes comprise about 100 to about 500 nucleotides. Probes that hybridize with centromeric DNA and locus-specific DNA or RNA are available commercially, for example, from Vysis, Inc. (Downers Grove, Ill.), Molecular Probes, Inc. (Eugene, Oreg.) or from Cytocell (Oxfordshire, UK). Alternatively, probes can be made non-commercially from chromosomal or genomic DNA or other sources of nucleic acids through standard techniques. Examples of probes, labeling and hybridization methods are known in the art.

Several variations of FISH methods are known in the art and are suitable for use according to the methods of the disclosure, including single-molecule RNA FISH, Fiber FISH, Q-FISH, Flow-FISH, MA-FISH, break-away FISH, hybrid fusion-FISH, and multi-fluor FISH or mFISH.

Detection Reagents

In some aspects, provided herein are reagents for detecting one or more gene alterations provided herein (e.g., in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D), e.g., according to the methods of detection described herein. In some embodiments, a detection reagent provided herein comprises a nucleic acid molecule, e.g., a DNA, RNA, or mixed DNA/RNA molecule, comprising a nucleotide sequence which is complementary to a nucleotide sequence on a target nucleic acid.

Baits

Provided herein are baits suitable for the detection of one or more gene alterations provided herein, e.g., in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D.

In some embodiments, the bait comprises a capture nucleic acid molecule configured to hybridize to a target nucleic acid molecule, e.g., a target nucleic acid molecule comprising nucleotide sequences of one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D.

In some embodiments, the capture nucleic acid molecule is configured to hybridize to a nucleic acid molecule encoding one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D.

In some embodiments, the capture nucleic acid molecule is configured to hybridize to a fragment of a nucleic acid molecule encoding one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D. In some embodiments, the fragment comprises (or is) between about 5 and about 25 nucleotides, between about 5 and about 300 nucleotides, between about 100 and about 300 nucleotides, between about 130 and about 230 nucleotides, or between about 150 and about 200 nucleotides. In some embodiments, the fragment comprises (or is) about 100 nucleotides, about 125 nucleotides, about 150 nucleotides, about 175 nucleotides, about 200 nucleotides, about 225 nucleotides, about 250 nucleotides, about 275 nucleotides, or about 300 nucleotides in length.

In some embodiments, the capture nucleic acid molecule is about 100 nucleotides, about 125 nucleotides, about 150 nucleotides, about 175 nucleotides, about 200 nucleotides, about 225 nucleotides, about 250 nucleotides, about 275 nucleotides, or about 300 nucleotides in length. In some embodiments, the capture nucleic acid molecule is between about 5 and about 25 nucleotides in length, between about 5 and about 300 nucleotides in length, between about 100 and about 300 nucleotides in length, between about 130 and about 230 nucleotides in length, or between about 150 and about 200 nucleotides in length.

In some embodiments, the gene alteration is a gene fusion or a rearrangement, and the capture nucleic acid molecule is configured to hybridize to the breakpoint that results in the gene fusion or rearrangement, and may further hybridize to between about 10 and about 100 nucleotides or more, e.g., any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides flanking either side of the breakpoint.

In some embodiments, the capture nucleic acid molecule is a DNA, RNA, or a DNA/RNA molecule. In some embodiments, the capture nucleic acid molecule comprises any of between about 50 and about 1000 nucleotides, between about 50 and about 500 nucleotides, between about 100 and about 500 nucleotides, between about 100 and about 300 nucleotides, between about 130 and about 230 nucleotides, or between about 150 and about 200 nucleotides. In some embodiments, the capture nucleic acid molecule comprises any of between about 50 nucleotides and about 100 nucleotides, about 100 nucleotides and about 150 nucleotides, about 150 nucleotides and about 200 nucleotides, about 200 nucleotides and about 250 nucleotides, about 250 nucleotides and about 300 nucleotides, about 300 nucleotides and about 350 nucleotides, about 350 nucleotides and about 400 nucleotides, about 400 nucleotides and about 450 nucleotides, about 450 nucleotides and about 500 nucleotides, about 500 nucleotides and about 550 nucleotides, about 550 nucleotides and about 600 nucleotides, about 600 nucleotides and about 650 nucleotides, about 650 nucleotides and about 700 nucleotides, about 700 nucleotides and about 750 nucleotides, about 750 nucleotides and about 800 nucleotides, about 800 nucleotides and about 850 nucleotides, about 850 nucleotides and about 900 nucleotides, about 900 nucleotides and about 950 nucleotides, or about 950 nucleotides and about 1000 nucleotides. In some embodiments, the capture nucleic acid molecule comprises about 150 nucleotides. In some embodiments, the capture nucleic acid molecule is about 150 nucleotides.

In some embodiments, a bait provided herein comprises a DNA, RNA, or a DNA/RNA molecule. In some embodiments, a bait provided herein includes a label or a tag. In some embodiments, the label or tag is a radiolabel, a fluorescent label, an enzymatic label, a sequence tag, biotin, or another ligand. In some embodiments, a bait provided herein includes a detection reagent such as a fluorescent marker. In some embodiments, a bait provided herein includes (e.g., is conjugated to) an affinity tag, e.g., that allows capture and isolation of a hybrid formed by a bait and a nucleic acid hybridized to the bait. In some embodiments, the affinity tag is an antibody, an antibody fragment, biotin, or any other suitable affinity tag or reagent known in the art. In some embodiments, a bait is suitable for solution phase hybridization.

Baits can be produced and used according to methods known in the art, e.g., as described in WO2012092426A1, incorporated herein by reference. For example, biotinylated RNA baits can be produced by obtaining a pool of synthetic long oligonucleotides, originally synthesized on a microarray, and amplifying the oligonucleotides to produce the bait sequences. In some embodiments, the baits are produced by adding an RNA polymerase promoter sequence at one end of the bait sequences, and synthesizing RNA sequences using RNA polymerase. In one embodiment, libraries of synthetic oligodeoxynucleotides can be obtained from commercial suppliers, such as Agilent Technologies, Inc., and amplified using known nucleic acid amplification methods.

In some embodiments, a bait provided herein comprises a target-specific bait sequence (e.g., a capture nucleic acid molecule described herein) and universal tails on each end. In some embodiments, a bait provided herein comprises an oligonucleotide comprising about 200 nucleotides, of which about 170 nucleotides are target-specific (e.g., a capture nucleic acid molecule described herein) and the other 30 nucleotides (e.g., 15 nucleotides on each end of the bait) are universal arbitrary tails, e.g., suitable for PCR amplification.

In some embodiments, a bait provided herein hybridizes to a nucleotide sequence comprising a gene alteration described herein, e.g., an alteration in PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D. In some embodiments, a bait provided herein hybridizes to a nucleotide sequence comprising a nucleotide sequence in an intron or an exon of one gene of a fusion or rearrangement described herein (e.g., a KMT2D rearrangement described herein), in an intron or an exon of the other gene of a fusion or rearrangement described herein (e.g., a KMT2D rearrangement described herein), or a breakpoint joining the introns and/or exons (e.g., a KMT2D rearrangement described herein).

The baits described herein can be used for selection of exons and short target sequences. In some embodiments, a bait is between about 100 nucleotides and 300 nucleotides. In some embodiments, a bait is between about 130 nucleotides and 230 nucleotides. In some embodiments, a bait is between about 150 nucleotides and 200 nucleotides. In some embodiments, the target-specific sequences in the baits, e.g., a capture nucleic acid molecule described herein, e.g., for selection of exons and short target sequences, are between about 40 nucleotides and 1000 nucleotides. In some embodiments, the target-specific sequence e.g., a capture nucleic acid molecule described herein, is between about 70 nucleotides and about 300 nucleotides. In some embodiments, the target-specific sequence, e.g., a capture nucleic acid molecule described herein, is between about 100 nucleotides and about 200 nucleotides. In some embodiments, the target-specific sequence, e.g., a capture nucleic acid molecule described herein, is between about 120 nucleotides and about 170 nucleotides.

In some embodiments, a bait of the disclosure distinguishes a nucleic acid, e.g., a genomic or transcribed nucleic acid, e.g., a cDNA or RNA, having a gene alteration described herein (e.g., an alteration in PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D), from a reference nucleotide sequence, e.g., a nucleotide sequence not having the gene alteration.

In some embodiments, the bait hybridizes to a nucleotide sequence encoding an alteration described herein (e.g., an alteration in PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D) and a sequence on either side of the alteration (e.g., any of 1, 2, 3, 4, 5, 6, 7, 8, 9, or 10 nucleotides on either side of the alteration, or any of between about 1 and about 5, about 5 and about 10, about 10 and about 15, about 15 and about 20, about 20 and about 25, about 25 and about 30, about 30 and about 35, about 35 and about 40, about 40 and about 45, about 45 and about about 50 and about 55, about 55 and about 60, about 60 and about 65, about 70 and about 75, about 75 and about 80, about 80 and about 85, about 85 and about 90, about 90 and about 95, or about and about 100, or more nucleotides on either side of the alteration). In some embodiments, the bait hybridizes to a breakpoint resulting in a gene fusion or rearrangement described herein (e.g., a KMT2D rearrangement), and a sequence on either side of the breakpoint (e.g., any of 1, 2, 3, 4, 5, 6, 7, 8, 9, or 10 nucleotides on either side of the breakpoint, or any of between about 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about about 75 and about 80, about 80 and about 85, about 85 and about 90, about 90 and about 95, or about 95 and about 100, or more nucleotides on either side of the breakpoint).

Probes

Also provided herein are probes, e.g., nucleic acid molecules, suitable for the detection of one or more gene alterations provided herein, e.g., in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D.

In some embodiments, a probe provided herein comprises a nucleic acid sequence configured to hybridize to a target nucleic acid molecule, e.g., a target nucleic acid molecule comprising nucleotide sequences of one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D.

In some embodiments, the probe comprises a nucleic acid sequence configured to hybridize to a nucleic acid molecule encoding one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D.

In some embodiments, the probe comprises a nucleic acid sequence configured to hybridize to a fragment or portion of a nucleic acid molecule encoding one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D. In some embodiments, the fragment or portion comprises between about 5 and about 25 nucleotides, between about 5 and about 300 nucleotides, between about 100 and about 300 nucleotides, between about 130 and about 230 nucleotides, or between about 150 and about 200 nucleotides.

In some embodiments, the probe comprises a nucleic acid sequence configured to hybridize to a nucleic acid molecule encoding an alteration described herein, e.g., an alteration in PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D, e.g., plus or minus any of between about 10 and about 20, about 20 and about 30, about 30 and about 40, about 40 and about 50, about 50 and about 60, about 60 and about 70, about 70 and about 80, about 80 and about 90, or about and about 100, or more nucleotides. In some embodiments, the probe comprises a nucleic acid sequence that hybridizes to a breakpoint resulting in a gene fusion or rearrangement provided herein (e.g., a KMT2D rearrangement described herein), e.g., plus or minus any of between about 10 and about 20, about 20 and about 30, about 30 and about 40, about 40 and about 50, about 50 and about about 60 and about 70, about 70 and about 80, about 80 and about 90, or about 90 and about 100, or more nucleotides. In some embodiments, the probe comprises a nucleic acid sequence configured to hybridize to a nucleotide sequence comprising a nucleotide sequence in an intron or an exon of one gene of a fusion or rearrangement described herein, or in a breakpoint resulting in a gene fusion or rearrangement described herein (e.g., plus or minus any of between about 10 and about 12, about 12 and about 15, about 15 and about 17, about 17 and about 20, about 20 and about 25, or about 25 and about 30, or more nucleotides). In some embodiments, the probe comprises a nucleic acid sequence configured to hybridize to a breakpoint joining an intron or an exon of one gene of a fusion or rearrangement described herein and an intron or an exon of the other gene of a fusion or rearrangement described herein (e.g., plus or minus any of between about 10 and about 12, about 12 and about 15, about 15 and about 17, about 17 and about 20, about 20 and about 25, or about 25 and about 30, or more nucleotides).

In some embodiments, the probe comprises a nucleic acid molecule which is a DNA, RNA, or a DNA/RNA molecule. In some embodiments, the probe comprises a nucleic acid molecule comprising any of between about 10 and about 20 nucleotides, between about 12 and about 20 nucleotides, between about 10 and about 1000 nucleotides, between about 50 and about 500 nucleotides, between about 100 and about 500 nucleotides, between about 100 and about 300 nucleotides, between about 130 and about 230 nucleotides, or between about 150 and about 200 nucleotides. In some embodiments, the probe comprises a nucleic acid molecule comprising any of about 10 nucleotides, about 11 nucleotides, about 12 nucleotides, about 13 nucleotides, about 14 nucleotides, about 15 nucleotides, about 16 nucleotides, about 17 nucleotides, about 18 nucleotides, about 19 nucleotides, about 20 nucleotides, about 21 nucleotides, about 22 nucleotides, about 23 nucleotides, about 24 nucleotides, about 25 nucleotides, about 26 nucleotides, about 27 nucleotides, about 28 nucleotides, about 29 nucleotides, or about 30 nucleotides. In some embodiments, the probe comprises a nucleic acid molecule comprising any of between about 40 nucleotides and about 50 nucleotides, about 50 nucleotides and about 100 nucleotides, about 100 nucleotides and about 150 nucleotides, about 150 nucleotides and about 200 nucleotides, about 200 nucleotides and about 250 nucleotides, about 250 nucleotides and about 300 nucleotides, about 300 nucleotides and about 350 nucleotides, about 350 nucleotides and about 400 nucleotides, about 400 nucleotides and about 450 nucleotides, about 450 nucleotides and about 500 nucleotides, about 500 nucleotides and about 550 nucleotides, about 550 nucleotides and about 600 nucleotides, about 600 nucleotides and about 650 nucleotides, about 650 nucleotides and about 700 nucleotides, about 700 nucleotides and about 750 nucleotides, about 750 nucleotides and about 800 nucleotides, about 800 nucleotides and about 850 nucleotides, about 850 nucleotides and about 900 nucleotides, about 900 nucleotides and about 950 nucleotides, or about 950 nucleotides and about 1000 nucleotides. In some embodiments, the probe comprises a nucleic acid molecule comprising between about 12 and about 20 nucleotides. In some embodiments, the probe comprises a nucleic acid molecule comprising about 17 nucleotides. In some embodiments, the probe comprises a nucleic acid molecule comprising about 18 nucleotides. In some embodiments, the probe comprises a nucleic acid molecule comprising about 19 nucleotides. In some embodiments, the probe comprises a nucleic acid molecule comprising about 20 nucleotides.

In some embodiments, a probe provided herein includes a label or a tag. In some embodiments, the label or tag is a radiolabel (e.g., a radioisotope), a fluorescent label (e.g., a fluorescent compound), an enzymatic label, an enzyme co-factor, a sequence tag, biotin, or another ligand. In some embodiments, a probe provided herein includes a detection reagent such as a fluorescent marker. In some embodiments, a probe provided herein includes (e.g., is conjugated to) an affinity tag, e.g., that allows capture and isolation of a hybrid formed by a probe and a nucleic acid hybridized to the probe. In some embodiments, the affinity tag is an antibody, an antibody fragment, biotin, or any other suitable affinity tag or reagent known in the art. In some embodiments, a probe is suitable for solution phase hybridization.

In some embodiments, probes provided herein may be used according to the methods of detection of one or more gene alterations provided herein, e.g., in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D. For example, a probe provided herein may be used for detecting one or more gene alterations provided herein, e.g., in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D, in sample, e.g., a sample obtained from an individual. In some embodiments, the probe may be used for identifying cells or tissues which express one or more genes comprising one or more alterations provided herein, e.g., by measuring levels of the one or more genes comprising the one or more alterations. In some embodiments, the probe may be used for detecting levels of one or more genes comprising one or more alterations provided herein, e.g., mRNA levels, in a sample of cells from an individual.

In some embodiments, a probe provided herein specifically hybridizes to a nucleic acid comprising a gene alteration described herein (e.g., a gene alteration in PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D). In some embodiments, a probe provided herein specifically hybridizes to a nucleic acid comprising a rearrangement (e.g., a fusion, a deletion, inversion, insertion, duplication, or other rearrangement) resulting in a rearrangement described herein, e.g., a KMT2D rearrangement.

In some embodiments, a probe of the disclosure distinguishes a nucleic acid, e.g., a genomic or transcribed nucleic acid, e.g., a cDNA or RNA, having a gene alteration described herein, from a reference nucleotide sequence, e.g., a nucleotide sequence not having the gene alteration.

Also provided herein are isolated pairs of allele specific probes, wherein, for example, the first probe of the pair specifically hybridizes to a nucleic acid molecule comprising nucleotide sequences of one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D and comprising an alteration described herein, and the second probe of the pair specifically hybridizes to a corresponding wild type sequence (e.g., a nucleic acid molecule comprising wild type nucleotide sequences of one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D). Probe pairs can be designed and produced for any of the alterations described herein and are useful in detecting a somatic mutation in a sample. In some embodiments, a first probe of a pair specifically hybridizes to a mutation (e.g., an alteration described herein), and a second probe of a pair specifically hybridizes to a sequence upstream or downstream of the mutation.

In some embodiments, one or more probes provided herein are suitable for use in in situ hybridization methods, e.g., as described above, such as FISH.

Chromosomal probes, e.g., for use in the FISH methods described herein, are typically about to about 10⁵ nucleotides. Longer probes typically comprise smaller fragments of about 100 to about 500 nucleotides. Probes that hybridize with centromeric DNA and locus-specific DNA are available commercially, for example, from Vysis, Inc. (Downers Grove, Ill.), Molecular Probes, Inc. (Eugene, Oreg.) or from Cytocell (Oxfordshire, UK). Alternatively, probes can be made non-commercially from chromosomal or genomic DNA through standard techniques. For example, sources of DNA that can be used include genomic DNA, cloned DNA sequences, somatic cell hybrids that contain one, or a part of one, chromosome (e.g., human chromosome) along with the normal chromosome complement of the host, and chromosomes purified by flow cytometry or microdissection. The region of interest can be isolated through cloning, or by site-specific amplification via the polymerase chain reaction (PCR). Probes of the disclosure may also hybridize to RNA molecules, e.g., mRNA, such as an RNA comprising one or more gene alterations provided herein, e.g., in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D.

In some embodiments, probes, such as probes for use in the FISH methods described herein, are used for determining whether a cytogenetic abnormality is present in one or more cells, e.g., in a region of a chromosome or an RNA bound by one or probes provided herein, such as a cytogenetic abnormality that results in a gene alteration provided herein, e.g., in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D. Examples of such cytogenetic abnormalities include, without limitation, deletions (e.g., deletions of entire chromosomes or deletions of fragments of one or more chromosomes), duplications (e.g., of entire chromosomes, or of regions smaller than an entire chromosome), translocations (e.g., non-reciprocal translocations, balanced translocations), intra-chromosomal inversions, point mutations, deletions, gene copy number changes, germ-line mutations, and gene expression level changes.

In some embodiments, probes, such as probes for use in the FISH methods described herein, are labeled such that a chromosomal region or a region on an RNA to which they hybridize can be detected. Probes typically are directly labeled with a fluorophore, allowing the probe to be visualized without a secondary detection molecule. Probes can also be labeled by nick translation, random primer labeling or PCR labeling. Labeling is done using either fluorescent (direct)-or haptene (indirect)-labeled nucleotides. Representative, non-limiting examples of labels include: AMCA-6-dUTP, CascadeBlue-4-dUTP, Fluorescein-12-dUTP, Rhodamine-6-dUTP, TexasRed-6-dUTP, Cy3-6-dUTP, Cy5-dUTP, Biotin(BIO)-11-dUTP, Digoxygenin(DIG)-11-dUTP and Dinitrophenyl (DNP)-11-dUTP. Probes can also be indirectly labeled with biotin or digoxygenin, or labeled with radioactive isotopes such as ³²P and ³H, and secondary detection molecules or further processing is performed to visualize the probes. For example, a probe labeled with biotin can be detected by avidin conjugated to a detectable marker, e.g., avidin can be conjugated to an enzymatic marker such as alkaline phosphatase or horseradish peroxidase. Enzymatic markers can be detected in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase. Probes can also be prepared such that a fluorescent or other label is added after hybridization of the probe to its target (e.g., on a chromosome), to detect the probe hybridized to a chromosome. For example, probes can be used that have antigenic molecules incorporated into the DNA. After hybridization, these antigenic molecules are detected using specific antibodies reactive with the antigenic molecules. Such antibodies can themselves incorporate a fluorochrome, or can be detected using a second antibody with a bound fluorochrome.

For fluorescent probes used in fluorescence in situ hybridization (FISH) techniques, e.g., as described herein, fluorescence can be viewed with a fluorescence microscope equipped with an appropriate filter for each fluorophore, or by using dual or triple band-pass filter sets to observe multiple fluorophores. Alternatively, techniques such as flow cytometry can be used to examine the hybridization pattern of the chromosomal probes.

In some embodiments, the probe hybridizes to a nucleotide sequence encoding an alteration described herein (e.g., an alteration in PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D) and a sequence on either side of the alteration (e.g., any of 1, 2, 3, 4, 5, 6, 7, 8, 9, or 10 nucleotides on either side of the alteration, or any of between about 1 and about 5, about 5 and about 10, about 10 and about 15, about 15 and about 20, about 20 and about 25, about 25 and about 30, about 30 and about 35, about 35 and about 40, about 40 and about 45, about 45 and about about 50 and about 55, about 55 and about 60, about 60 and about 65, about 70 and about 75, about 75 and about 80, about 80 and about 85, about 85 and about 90, about 90 and about 95, or about and about 100, or more nucleotides on either side of the alteration). In some embodiments, the probe hybridizes to a breakpoint resulting in a gene fusion or rearrangement described herein (e.g., a KMT2D rearrangement), and a sequence on either side of the breakpoint (e.g., any of 1, 2, 3, 4, 5, 6, 7, 8, 9, or 10 nucleotides on either side of the breakpoint, or any of between about 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about about 75 and about 80, about 80 and about 85, about 85 and about 90, about 90 and about 95, or about 95 and about 100, or more nucleotides on either side of the breakpoint).

Oligonucleotides

In some aspects, provided herein are oligonucleotides, e.g., useful as primers. In some embodiments, an oligonucleotide, e.g., a primer, provided herein comprises a nucleic acid sequence configured to hybridize to a target nucleic acid molecule, e.g., a target nucleic acid molecule comprising nucleotide sequences of one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D.

In some embodiments, an oligonucleotide, e.g., a primer, provided herein is configured to hybridize to a nucleic acid molecule encoding one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D.

In some embodiments, an oligonucleotide, e.g., a primer, provided herein is configured to hybridize to a fragment of a nucleic acid molecule encoding one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D.

In some embodiments, the oligonucleotide comprises a nucleic acid sequence configured to hybridize to a nucleotide sequence encoding a gene alteration provided herein (e.g., an alteration in PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D), e.g., plus or minus any of between about 10 and about 12, about 12 and about 15, about 15 and about 17, about 17 and about 20, about 20 and about 25, or about 25 and about 30, or more nucleotides. In some embodiments, the oligonucleotide comprises a nucleic acid sequence configured to hybridize to a breakpoint resulting in a gene fusion or rearrangement provided herein (e.g., a KMT2D rearrangement provided herein), e.g., plus or minus any of between about 10 and about 12, about 12 and about 15, about 15 and about 17, about 17 and about 20, about 20 and about 25, or about 25 and about 30, or more nucleotides.

In some embodiments, the oligonucleotide comprises a nucleic acid sequence configured to hybridize to a nucleotide sequence comprising a nucleotide sequence in an intron or an exon of one gene of a fusion or rearrangement described herein, or in a breakpoint resulting in a gene fusion or rearrangement described herein (e.g., plus or minus any of between about 10 and about 12, about 12 and about 15, about 15 and about 17, about 17 and about 20, about 20 and about 25, or about 25 and about 30, or more nucleotides). In some embodiments, the oligonucleotide comprises a nucleic acid sequence configured to hybridize to a breakpoint joining an intron or an exon of one gene of a fusion or rearrangement described herein and an intron or an exon of the other gene of a fusion or rearrangement described herein (e.g., plus or minus any of between about 10 and about 12, about 12 and about 15, about 15 and about 17, about 17 and about 20, about 20 and about 25, or about 25 and about 30, or more nucleotides).

In some embodiments, the oligonucleotide comprises a nucleic acid sequence corresponding to a nucleic acid molecule encoding one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D, e.g., and comprising one or more alterations described herein. In some embodiments, the oligonucleotide comprises a nucleic acid sequence corresponding to a fragment or a portion of one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D, e.g., and comprising one or more alterations described herein. In some embodiments, the fragment or portion comprises between about 10 and about 30 nucleotides, between about 12 and about 20 nucleotides, or between about 12 and about 17 nucleotides.

In some embodiments, the oligonucleotide comprises a nucleic acid sequence complementary to one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D, e.g., and comprising one or more alterations described herein. In some embodiments, the oligonucleotide comprises a nucleic acid sequence complementary to a fragment or a portion of one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D, e.g., and comprising one or more alterations described herein. In some embodiments, the fragment or portion comprises between about 10 and about 30 nucleotides, between about 12 and about 20 nucleotides, or between about 12 and about 17 nucleotides.

In some embodiments, the oligonucleotide comprises a nucleic acid sequence corresponding to a gene alteration provided herein (e.g., an alteration in PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D), e.g., plus or minus any of between about 10 and about 12, about 12 and about 15, about 15 and about 17, about 17 and about 20, about 20 and about 25, or about 25 and about 30, or more nucleotides. In some embodiments, the oligonucleotide comprises a nucleic acid sequence corresponding to a breakpoint between one gene of a fusion or rearrangement provided herein and the other gene of a fusion or rearrangement provided herein (e.g., a KMT2D rearrangement provided herein), e.g., plus or minus any of between about 10 and about 12, about 12 and about 15, about 15 and about 17, about 17 and about 20, about 20 and about 25, or about 25 and about 30, or more nucleotides.

In some embodiments, the oligonucleotide comprises a nucleic acid sequence complementary to a gene alteration provided herein (e.g., an alteration in PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D), e.g., plus or minus any of between about 10 and about 12, about 12 and about 15, about 15 and about 17, about 17 and about 20, about 20 and about 25, or about 25 and about 30, or more nucleotides. In some embodiments, the oligonucleotide comprises a nucleic acid sequence complementary to a breakpoint between one gene of a fusion or rearrangement provided herein and the other gene of a fusion or rearrangement provided herein (e.g., a KMT2D rearrangement provided herein), e.g., plus or minus any of between about 10 and about 12, about 12 and about 15, about 15 and about 17, about 17 and about 20, about 20 and about 25, or about 25 and about 30, or more nucleotides.

In some embodiments, the oligonucleotide comprises a nucleic acid sequence complementary to a nucleotide sequence in an intron or an exon of one gene of a fusion or rearrangement provided herein, or to a breakpoint joining the introns or exons of one gene of a fusion or rearrangement provided herein and the other gene of a fusion or rearrangement provided herein (e.g., plus or minus any of between about 10 and about 12, about 12 and about 15, about 15 and about 17, about 17 and about 20, about 20 and about 25, or about 25 and about 30, or more nucleotides). In some embodiments, the oligonucleotide comprises a nucleic acid sequence corresponding to a nucleotide sequence in an intron or an exon of one gene of a fusion or rearrangement provided herein, or to a breakpoint joining the introns or exons of one gene of a fusion or rearrangement provided herein and the other gene of a fusion or rearrangement provided herein (e.g., plus or minus any of between about and about 12, about 12 and about 15, about 15 and about 17, about 17 and about 20, about 20 and about 25, or about 25 and about 30, or more nucleotides).

In some embodiments, an oligonucleotide, e.g., a primer, provided herein comprises a nucleotide sequence that is sufficiently complementary to its target nucleotide sequence such that the oligonucleotide specifically hybridizes to a nucleic acid molecule comprising the target nucleotide sequence, e.g., under high stringency conditions. In some embodiments, an oligonucleotide, e.g., a primer, provided herein comprises a nucleotide sequence that is sufficiently complementary to its target nucleotide sequence such that the oligonucleotide specifically hybridizes to a nucleic acid molecule comprising the target nucleotide sequence under conditions that allow a polymerization reaction (e.g., PCR) to occur.

In some embodiments, an oligonucleotide, e.g., a primer, provided herein may be useful for initiating DNA synthesis via PCR (polymerase chain reaction) or a sequencing method.

In some embodiments, the oligonucleotide may be used to amplify a nucleic acid molecule comprising one or more alterations described herein (e.g., in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D), e.g., using PCR. In some embodiments, the oligonucleotide may be used to sequence a nucleic acid molecule comprising one or more alterations described herein, e.g., in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D.

In some embodiments, pairs of oligonucleotides, e.g., pairs of primers, are provided herein, which are configured to hybridize to a nucleic acid molecule comprising one or more alterations described herein (e.g., in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D) for use in directing amplification of the nucleic acid molecule or a fragment thereof, e.g., using a PCR reaction.

In some embodiments, an oligonucleotide, e.g., a primer, provided herein is a single stranded nucleic acid molecule, e.g., for use in sequencing or amplification methods. In some embodiments, an oligonucleotide provided herein is a double stranded nucleic acid molecule. In some embodiments, a double stranded oligonucleotide is treated, e.g., denatured, to separate its two strands prior to use, e.g., in sequencing or amplification methods. Oligonucleotides provided herein comprise a nucleotide sequence of sufficient length to hybridize to their target, and to prime the synthesis of extension products, e.g., during PCR or sequencing.

In some embodiments, an oligonucleotide provided herein comprises at least three deoxyribonucleotides or ribonucleotides. In some embodiments, an oligonucleotide provided herein comprises at least about eight deoxyribonucleotides or ribonucleotides. In some embodiments, an oligonucleotide provided herein comprises at least about 20 deoxyribonucleotides or ribonucleotides. In some embodiments, an oligonucleotide provided herein comprises at least about 30 deoxyribonucleotides or ribonucleotides. In some embodiments, an oligonucleotide provided herein comprises between about 10 and about 30 deoxyribonucleotides or ribonucleotides. In some embodiments, an oligonucleotide provided herein comprises between about 10 and about 25 deoxyribonucleotides or ribonucleotides. In some embodiments, an oligonucleotide provided herein comprises between about 10 and about 20 deoxyribonucleotides or ribonucleotides. In some embodiments, an oligonucleotide provided herein comprises between about 10 and about 15 deoxyribonucleotides or ribonucleotides. In some embodiments, an oligonucleotide provided herein comprises between about 12 and about 20 deoxyribonucleotides or ribonucleotides. In some embodiments, an oligonucleotide provided herein comprises between about 17 and about 20 deoxyribonucleotides or ribonucleotides. In some embodiments, the length of an oligonucleotide provided herein is determined according to methods known in the art, e.g., based on factors such as the specific application (e.g., PCR, sequencing library preparation, sequencing), reaction conditions (e.g., buffers, temperature), and the nucleotide composition of the nucleotide sequence of the oligonucleotide or of its target complementary sequence.

In some embodiments, an oligonucleotide, e.g., a primer, of the disclosure distinguishes a nucleic acid, e.g., a genomic or transcribed nucleic acid, e.g., a cDNA or RNA, having an alteration described herein from a reference nucleotide sequence, e.g., a nucleotide sequence not having the alteration.

In one aspect, provided herein is a primer or primer set for amplifying a nucleic acid comprising an alteration (e.g., in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D) described herein. In another aspect, provided herein is a primer or primer set for amplifying a nucleic acid comprising a cytogenetic abnormality such as a chromosomal inversion, deletion, translocation, or duplication resulting in an alteration or in a gene fusion or rearrangement described herein. In certain aspects, provided herein are allele-specific oligonucleotides, e.g., primers, wherein a first oligonucleotide of a pair specifically hybridizes to a mutation (e.g., an alteration in a gene selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D), and a second oligonucleotide of a pair specifically hybridizes to a sequence upstream or downstream of the mutation.

In some embodiments, the oligonucleotide, e.g., the primer, hybridizes to a nucleotide sequence comprising an alteration described herein (e.g., in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D), and a sequence on either side of the alteration (e.g., any of 1, 2, 3, 4, 5, 6, 7, 8, 9, or 10 nucleotides on either side of the alteration, or any of between about 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about 75, about 75 and about 80, about 80 and about 85, about 85 and about 90, about 90 and about 95, or about 95 and about 100, or more nucleotides on either side of the alteration). In some embodiments, the oligonucleotide, e.g., the primer, hybridizes to a breakpoint resulting in a gene fusion or rearrangement described herein, and a sequence on either side of the breakpoint (e.g., any of 1, 2, 3, 4, 5, 6, 7, 8, 9, or 10 nucleotides on either side of the breakpoint, or any of between about 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about 75, about 75 and about 80, about 80 and about 85, about 85 and about 90, about 90 and about 95, or about 95 and about 100, or more nucleotides on either side of the breakpoint).

Detection of Gene Alterations in Proteins

In some embodiments, the methods provided herein comprise acquiring knowledge of an alteration in one or more genes in a sample from an individual. In some embodiments, the methods provided herein comprise detecting an alteration in one or more genes in a sample from an individual. In some embodiments, the one or more genes include PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D. In some embodiments, the acquiring knowledge comprises detecting the alteration in a sample obtained from an individual. In some embodiments, the alteration is detected in a polypeptide or a protein, such as a polypeptide or a protein encoded by a gene comprising the alteration, such as PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D. In some embodiments, an alteration is detected in a polypeptide or a protein, such as a polypeptide or a protein encoded by a nucleic acid (e.g., genomic DNA or fragments thereof, a cDNA or fragments thereof, or an RNA (e.g., an mRNA) or fragments thereof) encoding a gene, such as PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D, and comprising the alteration.

In some embodiments, the methods comprise acquiring knowledge of or detecting an alteration in a polypeptide or a protein encoded by a PIK3CA gene. In some embodiments, the alteration in PIK3CA comprises one or more amino acid substitutions in a PIK3CA polypeptide. In some embodiments, the one or more amino acid substitutions are at amino acid position E81, R108, K111, G118, N345, D350, E365, E418, C420, E453, P539, E542, E545, Q546, E726, E970, M1004, M1043, N1044, H1047, G1049, or any combination thereof, wherein the numbering of the residues is according to SEQ ID NO: 26 or according to the amino acid sequence of a human PIK3CA protein. In some embodiments, the one or more amino acid substitutions in a polypeptide encoded by the PIK3CA gene comprise a E81K, R108H, K111N, G118D, N345K, D350N, E365K, E418K, C420R, E453K, E453Q, P539R, E542K, E545K, E545A, E545Q, Q546R, Q546K, E726K, E970K, M1004I, M1043I, N1044K, H1047R, H1047L, or G1049R amino acid substitution, or any combination thereof, wherein the numbering of the residues is according to SEQ ID NO: 26 or according to the amino acid sequence of a human PIK3CA protein. In some embodiments, the alteration in PIK3CA comprises an alteration that results in a deletion of one or more amino acids in a polypeptide encoded by the PIK3CA gene. In some embodiments, the deletion of one or more amino acids in a polypeptide encoded by the PIK3CA gene comprises a deletion of amino acid residue E110 (E110del), wherein the numbering of the residues is according to SEQ ID NO: 26, or according to the amino acid sequence of a human PIK3CA protein.

In some embodiments, the methods comprise acquiring knowledge of or detecting an alteration in a polypeptide or a protein encoded by an ESR1 gene. In some embodiments, the alteration in ESR1 comprises one or more amino acid substitutions in an ESR1 polypeptide. In some embodiments, the one or more amino acid substitutions are at amino acid position E380, V418, S463, V533, L536, Y537, D538, or any combination thereof, wherein the numbering of the residues is according to SEQ ID NO: 29, or according to the amino acid sequence of a human ESR1 protein. In some embodiments, the one or more amino acid substitutions in a polypeptide encoded by the ESR1 gene comprise a E380Q, V418E, S463P, V533M, L536Q, L536H, L536P, L536R, Y537S, Y537N, Y537C, Y537D, or D538G amino acid substitution, or any combination thereof, wherein the numbering of the residues is according to SEQ ID NO: 29, or according to the amino acid sequence of a human ESR1 protein. In some embodiments, the alteration in ESR1 is an alteration resulting in a deletion of one or more amino acids in a polypeptide encoded by the ESR1 gene. In some embodiments, the deletion of one or more amino acids in the polypeptide encoded by the ESR1 gene is a deletion of amino acid V422 (V422del) and/or a deletion of amino acids V533-L536 (V533_L536del) in a polypeptide encoded by the ESR1 gene, wherein the numbering of the residues is according to SEQ ID NO: 29, or according to the amino acid sequence of a human ESR1 protein.

In some embodiments, the methods comprise acquiring knowledge of or detecting an alteration in a polypeptide or a protein encoded by a KRAS gene. In some embodiments, the alteration in KRAS comprises one or more amino acid substitutions in a KRAS polypeptide. In some embodiments, the one or more amino acid substitutions are at amino acid position G12, G13, L19, Q61, A146, K147, F156, or any combination thereof, wherein the numbering of the residues is according to SEQ ID NO: 33, or according to the amino acid sequence of a human KRAS protein. In some embodiments, the one or more amino acid substitutions in a polypeptide encoded by the KRAS gene comprise a G12V, G12D, G12A, G12R, G12S, G12C, G12L, G13D, L19F, Q61H, Q61K, Q61E, A146T, K147N, or F156L amino acid substitution, or any combination thereof, wherein the numbering of the residues is according to SEQ ID NO: 33, or according to the amino acid sequence of a human KRAS protein.

In some embodiments, the methods comprise acquiring knowledge of or detecting an alteration in a polypeptide or a protein encoded by a ERBB2 gene. In some embodiments, the alteration in ERBB2 comprises one or more amino acid substitutions in an ERBB2 polypeptide. In some embodiments, the one or more amino acid substitutions are at amino acid position S310, S653, V659, R678, V697, E717, T733, L755, I767, D769, G776, V777, T798, V842, L869, or any combination thereof, wherein the numbering of the residues is according to SEQ ID NO: 34, or according to the amino acid sequence of a human ERBB2 protein. In some embodiments, the one or more amino acid substitutions in a polypeptide encoded by the ERBB2 gene comprise a S310F, S310Y, S653C, V659D, R678Q, V697L, E717K, T733I, L755S, L755P, I767M, D769N, D769Y, D769H, G776V, V777L, T798I, V842I, or L869R amino acid substitution, or any combination thereof, wherein the numbering of the residues is according to SEQ ID NO: 34, or according to the amino acid sequence of a human ERBB2 protein. In some embodiments, the alteration in ERBB2 results in a deletion of one or more amino acid residues in a polypeptide encoded by the ERBB2 gene. In some embodiments, the deletion of one or more amino acid residues in a polypeptide encoded by the ERBB2 gene is a deletion of amino acids L755-T759 (L755_T759del) and/or amino acids L755-E757 (L755_E757del) in a polypeptide encoded by the ERBB2 gene, wherein the numbering of the residues is according to SEQ ID NO: 34, or according to the amino acid sequence of a human ERBB2 protein. In some embodiments, the alteration in ERBB2 results in an insertion of one or more amino acid residues in a polypeptide encoded by the ERBB2 gene. In some embodiments, the insertion of one or more amino acid residues in a polypeptide encoded by the ERBB2 gene is an insertion of one or more amino acid residues between amino acid residues A775 and G776, and/or between amino acid residues P780 and Y781 in a polypeptide encoded by the ERBB2 gene, wherein the numbering of the residues is according to the amino acid sequence of SEQ ID NO: 34. In some embodiments, the insertion of one or more amino acid residues in a polypeptide encoded by the ERBB2 gene is an insertion of the amino acid sequence YVMA (SEQ ID NO: 51) between amino acid residues A775 and G776 (A775_G776insYVMA), and/or of the amino acid sequence GSP (SEQ ID NO: 52) between amino acid residues P780 and Y781 (P780_Y781insGSP) in a polypeptide encoded by the ERBB2 gene, wherein the numbering of the residues is according to SEQ ID NO: 34, or according to the amino acid sequence of a human ERBB2 protein.

In some embodiments, the methods comprise acquiring knowledge of or detecting an alteration in a polypeptide or a protein encoded by an FGFR2 gene. In some embodiments, the alteration in FGFR2 comprises one or more amino acid substitutions in an FGFR2 polypeptide. In some embodiments, the one or more amino acid substitutions are at amino acid position S252, P253, Y375, C382, M391, V395, M537, N549, K659, R664, or any combination thereof, wherein the numbering of the residues is according to SEQ ID NO: 38, or according to the amino acid sequence of a human FGFR2 protein. In some embodiments, the one or more amino acid substitutions in a polypeptide encoded by the FGFR2 gene comprise a S252W, P253R, Y375C, C382R, M391R, V395D, M537I, N549D, N549K, K659M, K659E, K659N, or R664W amino acid substitution, or any combination thereof, wherein the numbering of the residues is according to SEQ ID NO: 38, or according to the amino acid sequence of a human FGFR2 protein. In some embodiments, the alteration results in a frameshift in FGFR2 (e.g., in an mRNA sequence encoded by the FGFR2 gene and in the resulting amino acid sequence). In some embodiments, the frameshift is an S799fs*22 frameshift, wherein the numbering of the residues is according to SEQ ID NO: 38, or according to the amino acid sequence of a human FGFR2 protein.

In some embodiments, the methods comprise acquiring knowledge of or detecting an alteration in a polypeptide or a protein encoded by a BRAF gene. In some embodiments, the alteration in BRAF comprises one or more amino acid substitutions in a BRAF polypeptide. In some embodiments, the one or more amino acid substitutions are at amino acid position D380, G464, G466, S467, G469, L485, L584, E586, D594, V600, or any combination thereof, wherein the numbering of the residues is according to SEQ ID NO: 35 or according to the amino acid sequence of a human BRAF protein. In some embodiments, the one or more amino acid substitutions in a polypeptide encoded by the BRAF gene comprise a D380H, G464R, G466E, S467L, G469A, G469E, G469R, L485F, L584F, E586K, D594N, D594G, or V600E amino acid substitution, or any combination thereof, wherein the numbering of the residues is according to SEQ ID NO: 35, or according to the amino acid sequence of a human BRAF protein.

In some embodiments, an alteration is detected in a polypeptide or a protein using any suitable method known in the art, for example, using antibodies, mass spectrometry (e.g., tandem mass spectrometry), a reporter assay (e.g., a fluorescence-based assay), immunoblots such as a Western blot, immunoassays such as enzyme-linked immunosorbent assays (ELISA), immunohistochemistry, other immunological assays (e.g., fluid or gel precipitin reactions, immunodiffusion, immunoelectrophoresis, radioimmunoassay (RIA), immunofluorescent assays), and analytic biochemical methods (e.g., electrophoresis, capillary electrophoresis, high performance liquid chromatography (HPLC), thin layer chromatography (TLC), hyperdiffusion chromatography).

In some embodiments, a polypeptide or a protein comprising an alteration described herein can be distinguished from a reference polypeptide, e.g., a non-mutant or wild type polypeptide or protein, by reaction with a detection reagent, e.g., a substrate, e.g., a substrate for catalytic activity, or with an antibody or fragment thereof that reacts differentially with a mutant protein or polypeptide as compared to a reference protein or polypeptide.

In some embodiments, methods of detection of a polypeptide or a protein comprising an alteration are provided, comprising contacting a sample, e.g., a sample described herein, with a detection reagent, and determining if the polypeptide or protein comprising an alteration is present in the sample.

Nucleic Acid Samples

A variety of materials (such as tissues) can be the source of the nucleic acid samples used in the methods provided herein. For example, the source of the sample can be solid tissue as from a fresh, frozen and/or preserved organ, tissue sample, biopsy, resection, smear, or aspirate; blood or any blood constituents; bodily fluids such as cerebral spinal fluid, amniotic fluid, urine, saliva, sputum, peritoneal fluid or interstitial fluid; or cells from any time in gestation or development of an individual. In some embodiments, the source of the sample is blood or blood constituents. In some embodiments, the source of the sample is a tumor sample, e.g., from an ILC metastasis, such as a bone ILC metastasis, a female reproductive system ILC metastasis, a gastrointestinal ILC metastasis, a liver ILC metastasis, or a skin ILC metastasis. In some embodiments, the sample is or comprises biological tissue or fluid. In some embodiments, the sample can contain compounds that are not naturally intermixed with the tissue in nature, such as preservatives, anticoagulants, buffers, fixatives, nutrients, antibiotics or the like. In some embodiments, a nucleic acid sample comprises genomic or subgenomic DNA fragments. In some embodiments, a nucleic acid sample comprises RNA, such as mRNA isolated from a sample, e.g., a tumor sample, a normal adjacent tissue (NAT) sample, a tissue sample, or a blood sample obtained from an individual. In some embodiments, the sample comprises cDNA derived from an mRNA sample or from a sample comprising mRNA. In some embodiments, the tissue is preserved as a frozen sample or as formaldehyde- or paraformaldehyde-fixed paraffin-embedded (FFPE) tissue preparation. For example, the sample can be embedded in a matrix, e.g., an FFPE block or a frozen sample.

In some embodiments, the sample comprises cell-free DNA (cfDNA). In some embodiments, the sample comprises cell-free RNA (cfRNA). In some embodiments, the sample comprises circulating tumor DNA (ctDNA), e.g., from an ILC metastasis, such as a bone ILC metastasis, a female reproductive system ILC metastasis, a gastrointestinal ILC metastasis, a liver ILC metastasis, or a skin ILC metastasis. In some embodiments, the sample comprises circulating tumor cells (CTCs), e.g., from an ILC metastasis, such as a bone ILC metastasis, a female reproductive system ILC metastasis, a gastrointestinal ILC metastasis, a liver ILC metastasis, or a skin ILC metastasis.

In some embodiments, the sample is or comprises bone marrow; a bone marrow aspirate; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as ductal lavages or bronchoalveolar lavages; aspirates; scrapings; bone marrow specimens; tissue biopsy specimens; surgical specimens; other body fluids, secretions, and/or excretions; and/or cells therefrom. In some embodiments, the sample is or comprises cells obtained from an individual.

In some embodiments, a sample is a primary sample obtained directly from a source of interest by any appropriate means. For example, in some embodiments, a primary biological sample is obtained by a method chosen from biopsy (e.g., fine needle aspiration or tissue biopsy), surgery, or collection of body fluid (e.g., blood, lymph, or feces). In some embodiments, the sample is a preparation that is obtained by processing (e.g., by removing one or more components of and/or by adding one or more agents to) a primary sample. Such as a processed sample may comprise, for example nucleic acids or proteins extracted from a sample or obtained by subjecting a primary sample to techniques such as amplification or reverse transcription of mRNA, or isolation and/or purification of certain components.

In some embodiments, the sample comprises one or more cells associated with a tumor, e.g., tumor cells or tumor-infiltrating lymphocytes (TIL), e.g., from an ILC metastasis, such as a bone ILC metastasis, a female reproductive system ILC metastasis, a gastrointestinal ILC metastasis, a liver ILC metastasis, or a skin ILC metastasis. In one embodiment, the sample includes one or more premalignant or malignant cells, e.g., from an ILC metastasis, such as a bone ILC metastasis, a female reproductive system ILC metastasis, a gastrointestinal ILC metastasis, a liver ILC metastasis, or a skin ILC metastasis.

In some embodiments, the sample is acquired from a cancer, such as an invasive lobular carcinoma (ILC), e.g., a metastatic ILC, such as a bone ILC metastasis, a female reproductive system ILC metastasis, a gastrointestinal ILC metastasis, a liver ILC metastasis, or a skin ILC metastasis. In some embodiments, the sample is acquired from a solid tumor, a soft tissue tumor or a metastatic lesion, e.g., an ILC metastatic lesion, such as a bone ILC metastasis, a female reproductive system ILC metastasis, a gastrointestinal ILC metastasis, a liver ILC metastasis, or a skin ILC metastasis. In other embodiments, the sample includes tissue or cells from a surgical margin. In another embodiment, the sample includes one or more circulating tumor cells (CTCs) (e.g., a CTC acquired from a blood sample). In an embodiment, the sample comprises a cell not associated with a tumor, e.g., a non-tumor cell or a peripheral blood lymphocyte.

In some embodiments, the sample comprises tumor nucleic acids, such as nucleic acids from a tumor or a cancer sample, e.g., genomic DNA, or cDNA derived from RNA, from a tumor or cancer sample, e.g., from an ILC metastasis, such as a bone ILC metastasis, a female reproductive system ILC metastasis, a gastrointestinal ILC metastasis, a liver ILC metastasis, or a skin ILC metastasis. In certain embodiments, a tumor nucleic acid sample is purified or isolated (e.g., it is removed from its natural state).

In some embodiments, the sample is a control nucleic acid sample or a reference nucleic acid sample, e.g., genomic DNA, or cDNA derived from RNA. In certain embodiments, the reference or control nucleic acid sample comprises a wild type or a non-mutated sequence. In certain embodiments, the reference nucleic acid sample is purified or isolated (e.g., it is removed from its natural state). In other embodiments, the reference nucleic acid sample is from a non-tumor sample, e.g., a blood control, a normal adjacent tumor (NAT), or any other non-cancerous sample from the same or a different subject.

Protein Samples

A variety of materials (such as tissues) can be the source of the protein samples used in the methods provided herein. For example, the source of the sample can be a solid tissue, e.g., from a fresh, frozen and/or preserved organ, tissue sample, biopsy (e.g., a tumor biopsy), resection, smear, or aspirate; blood or any blood constituents; bodily fluids such as cerebral spinal fluid, amniotic fluid, urine, saliva, sputum, peritoneal fluid or interstitial fluid; or cells such as tumor cells. In some embodiments, the source of the sample is blood or blood constituents. In some embodiments, the source of the sample is a tumor sample, e.g., from an ILC metastasis, such as a bone ILC metastasis, a female reproductive system ILC metastasis, a gastrointestinal ILC metastasis, a liver ILC metastasis, or a skin ILC metastasis. In some embodiments, the sample is or comprises biological tissue or fluid. In some embodiments, the sample is preserved as a frozen sample or as a formaldehyde- or paraformaldehyde-fixed paraffin-embedded (FFPE) tissue preparation. In some embodiments, the sample comprises circulating tumor cells (CTCs), e.g., from an ILC metastasis, such as a bone ILC metastasis, a female reproductive system ILC metastasis, a gastrointestinal ILC metastasis, a liver ILC metastasis, or a skin ILC metastasis.

In some embodiments, a protein sample for use according to the methods provided herein comprises proteins isolated or obtained from a solid tissue, e.g., from a fresh, frozen and/or preserved organ, tissue sample, biopsy (e.g., a tumor biopsy), resection, smear, or aspirate; from blood or any blood constituents; from bodily fluids such as cerebral spinal fluid, amniotic fluid, urine, saliva, sputum, peritoneal fluid or interstitial fluid; or from cells such as tumor cells. In some embodiments, the sample comprises proteins isolated or obtained from a preserved sample, such as a frozen sample or as a formaldehyde- or paraformaldehyde-fixed paraffin-embedded (FFPE) tissue preparation. In some embodiments, the sample comprises proteins isolated or obtained from circulating tumor cells (CTCs). In some embodiments, the sample can contain compounds that are not naturally intermixed with the tissue in nature such as preservatives, anticoagulants, buffers, fixatives, nutrients, antibiotics or the like.

In some embodiments, a sample is or comprises bone marrow; a bone marrow aspirate; blood; blood cells; ascites; tissue or fine needle biopsy samples; cell-containing body fluids; free floating nucleic acids; sputum; saliva; urine; cerebrospinal fluid; peritoneal fluid; pleural fluid; feces; lymph; gynecological fluids; skin swabs; vaginal swabs; oral swabs; nasal swabs; washings or lavages such as ductal lavages or bronchoalveolar lavages; aspirates; scrapings; bone marrow specimens; tissue biopsy specimens; surgical specimens; other body fluids, secretions, and/or excretions; and/or cells therefrom. In some embodiments, a biological sample is or comprises cells obtained from an individual.

In some embodiments, a sample is a primary sample obtained directly from a source of interest by any appropriate means. For example, in some embodiments, a primary biological sample is obtained by a method chosen from biopsy (e.g., fine needle aspiration or tissue biopsy), surgery, collection of body fluid (e.g., blood, lymph, or feces). In some embodiments, a sample is a preparation that is obtained by processing (e.g., by removing one or more components of and/or by adding one or more agents to) a primary sample. Such as a processed sample may comprise, for example proteins extracted from a sample or obtained by subjecting a primary sample to techniques such as isolation and/or purification of certain components.

In some embodiments, the sample is acquired from a cancer, such as an invasive lobular carcinoma (ILC), e.g., an ILC metastasis, such as a bone ILC metastasis, a female reproductive system ILC metastasis, a gastrointestinal ILC metastasis, a liver ILC metastasis, or a skin ILC metastasis. In some embodiments, the sample is acquired from a solid tumor, a soft tissue tumor or a metastatic lesion, e.g., an ILC metastatic lesion, such as a bone ILC metastasis, a female reproductive system ILC metastasis, a gastrointestinal ILC metastasis, a liver ILC metastasis, or a skin ILC metastasis. In other embodiments, the sample includes tissue or cells from a surgical margin. In another embodiment, the sample includes one or more circulating tumor cells (CTCs) (e.g., a CTC acquired from a blood sample). In an embodiment, the sample comprises a cell not associated with a tumor, e.g., a non-tumor cell or a peripheral blood lymphocyte.

In some embodiments, the sample comprises one or more cells associated with a tumor, e.g., tumor cells or tumor-infiltrating lymphocytes (TIL), e.g., from an ILC metastasis, such as a bone ILC metastasis, a female reproductive system ILC metastasis, a gastrointestinal ILC metastasis, a liver ILC metastasis, or a skin ILC metastasis. In some embodiments, the sample includes one or more premalignant or malignant cells. In some embodiments, the sample is acquired from a cancer described herein, e.g., from an ILC metastasis, such as a bone ILC metastasis, a female reproductive system ILC metastasis, a gastrointestinal ILC metastasis, a liver ILC metastasis, or a skin ILC metastasis.

Methods of Diagnosing or Assessing

In some aspects, provided herein are methods of diagnosing or assessing an ILC metastasis in an individual.

In some embodiments, the methods comprise acquiring knowledge that the ILC metastasis has a high tumor mutational burden, e.g., of at least about 10 mut/Mb. In some embodiments, the methods comprise detecting a high tumor mutational burden in an ILC metastasis, e.g., of at least about 10 mut/Mb. In some embodiments, the methods comprise measuring the level of tumor mutational burden in a sample, e.g., in a sample from an ILC tumor, obtained from an individual, e.g., according to the methods of detection of tumor mutational burden provided herein. In some embodiments, the methods further comprise providing a diagnosis or an assessment of tumor mutational burden in the sample. In some embodiments, the diagnosis or assessment identifies the ILC metastasis as likely to respond to an anti-cancer therapy, e.g., an immune checkpoint inhibitor provided herein. In some embodiments, the presence of a high tumor mutational burden, e.g., of at least about 10 mut/Mb, identifies the ILC metastasis as likely to respond to an anti-cancer therapy, e.g., an immune checkpoint inhibitor provided herein.

In some embodiments, the methods comprise acquiring knowledge that the ILC metastasis is PD-L1 positive. In some embodiments, the methods comprise detecting a PD-L1 positive ILC metastasis. In some embodiments, the methods comprise measuring the level of expression of PD-L1 in a sample, e.g., in a sample from an ILC tumor, obtained from an individual, e.g., according to the methods of measuring the level of expression of PD-L1 provided herein. In some embodiments, the methods further comprise providing a diagnosis or an assessment of the level of PD-L1 expression in the sample. In some embodiments, the diagnosis or assessment identifies the ILC metastasis as likely to respond to an anti-cancer therapy, e.g., an immune checkpoint inhibitor provided herein. In some embodiments, the presence of a PD-L1 positive status identifies the ILC metastasis as likely to respond to an anti-cancer therapy, e.g., an immune checkpoint inhibitor provided herein.

In some embodiments, the methods comprise acquiring knowledge that the ILC metastasis comprises an alteration in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D. In some embodiments, the methods comprise detecting an alteration in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D in a sample, e.g., in a sample from an ILC tumor, obtained from an individual, e.g., according to the methods of detection of gene alterations provided herein. In some embodiments, the methods further comprise providing a diagnosis or an assessment of the alteration in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D in the sample. In some embodiments, the diagnosis or assessment identifies the ILC metastasis as likely to respond to an anti-cancer therapy, e.g., an anti-cancer therapy provided herein. In some embodiments, the presence of an alteration in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D identifies the ILC metastasis as likely to respond to an anti-cancer therapy, e.g., an anti-cancer therapy provided herein. In some embodiments, the presence of an alteration in PIK3CA in a sample from an individual having an ILC metastasis identifies the individual as one who may benefit from a treatment comprising a kinase inhibitor, an AKT inhibitor, an EZH2 inhibitor, or an mTOR inhibitor. In some embodiments, the alteration comprises one or more of a substitution of one or more nucleotides, an insertion of one or more nucleotides, or a deletion of one or more nucleotides in PIK3CA. In some embodiments, the presence of an alteration in BRCA1 in a sample from an individual having an ILC metastasis identifies the individual as one who may benefit from a treatment comprising a PARP inhibitor, a Chk1/2 inhibitor, or a Wee1 inhibitor. In some embodiments, the presence of an alteration in BRCA2 in a sample from an individual having an ILC metastasis identifies the individual as one who may benefit from a treatment comprising a PARP inhibitor, a Chk1/2 inhibitor, or a Wee1 inhibitor. In some embodiments, the presence of an alteration in BRCA1 and BRCA2 in a sample from an individual having an ILC metastasis identifies the individual as one who may benefit from a treatment comprising a PARP inhibitor, a Chk1/2 inhibitor, or a Wee1 inhibitor. In some embodiments, the presence of an alteration in ESR1 in a sample from an individual having an ILC metastasis identifies the individual as one who may benefit from a treatment comprising a Selective Estrogen Receptor Modulator (SERM), an anti-estrogen, or an aromatase inhibitor. In some embodiments, the alteration comprises one or more of a substitution of one or more nucleotides, an insertion of one or more nucleotides, or a deletion of one or more nucleotides in ESR1. In some embodiments, the presence of an alteration in NF1 in a sample from an individual having an ILC metastasis identifies the individual as one who may benefit from a treatment comprising a kinase inhibitor, an mTOR inhibitor, an EGFR inhibitor, a glutaminase inhibitor, or a MEK inhibitor. In some embodiments, the alteration comprises one or more of a substitution of one or more nucleotides, an insertion of one or more nucleotides, or a deletion of one or more nucleotides in NF1. In some embodiments, the presence of an alteration in RB1 in a sample from an individual having an ILC metastasis identifies the individual as one who may benefit from a treatment comprising a SOX2 inhibitor, an EZH2 inhibitor, a chemotherapy, a checkpoint kinase (CHK) inhibitor, a CDC25 phosphatase inhibitor, a polo-like kinase (PLK) inhibitor, or an aurora kinase (AURK) inhibitor. In some embodiments, the alteration comprises one or more of a substitution of one or more nucleotides, an insertion of one or more nucleotides, or a deletion of one or more nucleotides in RB1. In some embodiments, the presence of an alteration in KRAS in a sample from an individual having an ILC metastasis identifies the individual as one who may benefit from a treatment comprising a kinase inhibitor, a farnesyltransferase inhibitor, a geranylgeranyltransferase inhibitor, a palmitoylation inhibitor, an inhibitor of methylation cleavage, a Raf inhibitor, a MEK inhibitor, an mTOR inhibitor, or an agent that inhibits the modification or post-translational processing of KRAS. In some embodiments, the presence of an alteration in ERBB2 in a sample from an individual having an ILC metastasis identifies the individual as one who may benefit from a treatment comprising a kinase inhibitor, a small molecule, an antibody or antibody fragment, a cellular immunotherapy, or a pan-ERBB inhibitor. In some embodiments, the kinase inhibitor is a multi-specific kinase inhibitor, a reversible HER2 inhibitor, an irreversible HER2 inhibitor, a pan-ERBB inhibitor, a dual HER2 inhibitor, a HER2-specific inhibitor, an EGFR inhibitor, or a dual EGFR/ERBB inhibitor. In some embodiments, the presence of an alteration in BRAF in a sample from an individual having an ILC metastasis identifies the individual as one who may benefit from a treatment comprising a kinase inhibitor. In some embodiments, the presence of an alteration in ARID1A in a sample from an individual having an ILC metastasis identifies the individual as one who may benefit from a treatment comprising a PARP inhibitor, a bromodomain-containing protein 4 (BRD4) inhibitor, an HDAC inhibitor, a PI3K inhibitor, an ATR inhibitor, an EZH2 inhibitor, an AKT inhibitor, a TrxR inhibitor, a GSH inhibitor, or an immune checkpoint inhibitor. In some embodiments, the alteration comprises one or more of a substitution of one or more nucleotides, an insertion of one or more nucleotides, or a deletion of one or more nucleotides in ARID1A. In some embodiments, the presence of an alteration in PTEN in a sample from an individual having an ILC metastasis identifies the individual as one who may benefit from a treatment comprising a PI3K inhibitor, an AKT inhibitor, an mTOR inhibitor, or a MET inhibitor. In some embodiments, the alteration is a PTEN deletion. In some embodiments, the presence of an alteration in FGFR2 in a sample from an individual having an ILC metastasis identifies the individual as one who may benefit from a treatment comprising a kinase inhibitor. In some embodiments, the presence of an alteration in SMAD4 in a sample from an individual having an ILC metastasis identifies the individual as one who may benefit from a treatment comprising a PARP inhibitor. In some embodiments, the presence of an alteration in PTPN11 in a sample from an individual having an ILC metastasis identifies the individual as one who may benefit from a treatment comprising a small molecule inhibitor or a kinase inhibitor. In some embodiments, the alteration comprises one or more of a substitution of one or more nucleotides, an insertion of one or more nucleotides, or a deletion of one or more nucleotides in PTPN11. In some embodiments, the presence of an alteration in TERT in a sample from an individual having an ILC metastasis identifies the individual as one who may benefit from a treatment comprising a small molecule inhibitor, a modified nucleotide or nucleoside analog, a G-quadruplex stabilizer, a heat shock protein (HSP) inhibitor, or a MYC inhibitor. In some embodiments, the alteration comprises one or more of a substitution of one or more nucleotides, an insertion of one or more nucleotides, or a deletion of one or more nucleotides in TERT. In some embodiments, the presence of an alteration in ALK in a sample from an individual having an ILC metastasis identifies the individual as one who may benefit from a treatment comprising a kinase inhibitor, a heat shock protein (HSP) inhibitor or a MYC inhibitor. In some embodiments, the alteration comprises one or more of a substitution of one or more nucleotides, an insertion of one or more nucleotides, or a deletion of one or more nucleotides in ALK. In some embodiments, the presence of an alteration in APC in a sample from an individual having an ILC metastasis identifies the individual as one who may benefit from a treatment comprising a beta-catenin inhibitor or an APC inhibitor.

Methods Selecting a Treatment

In some aspects, provided herein are methods of identifying an individual having an ILC metastasis, who may benefit from an anti-cancer therapy, e.g., an anti-cancer agent provided herein. In some embodiments, the methods comprise acquiring knowledge of or detecting one or more biomarkers provided herein in a sample obtained from the individual. In some embodiments, the presence of the one or more biomarkers identifies the individual as one who may benefit from a treatment comprising an anti-cancer therapy, e.g., an anti-cancer therapy provided herein. In some embodiments, the one or more biomarkers are detected in a sample from an individual according to any of the methods of detection provided herein.

In other aspects, provided herein are methods of identifying or selecting a treatment, a therapy, or one or more treatment options for an individual having an ILC metastasis, who may benefit from an anti-cancer therapy, e.g., an anti-cancer agent provided herein. In some embodiments, the methods comprise acquiring knowledge of or detecting one or more biomarkers provided herein in a sample obtained from the individual. In some embodiments, responsive to knowledge of the presence of one or more biomarkers in a sample from an individual having an ILC metastasis, the individual is classified as a candidate to receive a treatment comprising an anti-cancer therapy, such as an anti-cancer agent provided herein. In some embodiments, responsive to knowledge of the presence of one or more biomarkers in a sample from an individual having an ILC metastasis, the individual is classified or identified as likely to respond to a treatment comprising an anti-cancer therapy, such as an anti-cancer agent provided herein. In some embodiments, the one or more biomarkers are detected in a sample from an individual according to any of the methods of detection provided herein.

In some embodiments, the presence of a tumor mutational burden (TMB) of at least about 10 mutations/megabase (mut/Mb) in a sample from an individual having an ILC metastasis identifies the individual as one who may benefit from a treatment comprising an immune checkpoint inhibitor. In some embodiments, responsive to knowledge of the presence of a tumor mutational burden (TMB) of at least about 10 mutations/megabase (mut/Mb) in a sample from an individual having an ILC metastasis, the individual is classified as a candidate to receive a treatment comprising an immune checkpoint inhibitor. In some embodiments, responsive to knowledge of the presence of a tumor mutational burden (TMB) of at least about 10 mutations/megabase (mut/Mb) in a sample from an individual having an ILC metastasis, the individual is classified or identified as likely to respond to a treatment comprising an immune checkpoint inhibitor. In some embodiments, the methods further comprise generating a report, e.g., as described herein. In some embodiments, the report comprises one or more treatment options identified or selected, e.g., based, at least in part, on the detection of a tumor mutational burden (TMB) of at least about 10 mutations/megabase (mut/Mb) in a sample from the individual. In some embodiments, the report comprises one or more treatment options identified or selected, e.g., based, at least in part, on the presence of a tumor mutational burden (TMB) of at least about 10 mutations/megabase (mut/Mb) in a sample from the individual. In some embodiments, the report comprises one or more treatment options identified or selected, e.g., based, at least in part, on acquiring knowledge of the presence of a tumor mutational burden (TMB) of at least about 10 mutations/megabase (mut/Mb) in a sample from the individual. In some embodiments, the one or more treatment options include an anti-cancer therapy provided herein, e.g., an immune checkpoint inhibitor.

In some embodiments, the presence of a PD-L1-positive ILC metastasis in a sample from an individual having an ILC metastasis identifies the individual as one who may benefit from a treatment comprising an immune checkpoint inhibitor. In some embodiments, responsive to knowledge of the presence of a PD-L1-positive ILC metastasis in a sample from an individual having an ILC metastasis, the individual is classified as a candidate to receive a treatment comprising an immune checkpoint inhibitor. In some embodiments, responsive to knowledge of the presence of a PD-L1-positive ILC metastasis in a sample from an individual having an ILC metastasis, the individual is classified or identified as likely to respond to a treatment comprising an immune checkpoint inhibitor. In some embodiments, the methods further comprise generating a report, e.g., as described herein. In some embodiments, the report comprises one or more treatment options identified or selected, e.g., based, at least in part, on the detection of a PD-L1-positive ILC metastasis in a sample from the individual. In some embodiments, the report comprises one or more treatment options identified or selected, e.g., based, at least in part, on the presence of a PD-L1-positive ILC metastasis in a sample from the individual. In some embodiments, the report comprises one or more treatment options identified or selected, e.g., based, at least in part, on acquiring knowledge of the presence of a PD-L1-positive ILC metastasis in a sample from the individual. In some embodiments, the one or more treatment options include an anti-cancer therapy provided herein, e.g., an immune checkpoint inhibitor.

In some embodiments, the presence of an alteration in PIK3CA in a sample from an individual having an ILC metastasis identifies the individual as one who may benefit from a treatment comprising a kinase inhibitor, an AKT inhibitor, an EZH2 inhibitor, or an mTOR inhibitor. In some embodiments, responsive to knowledge of the presence of an alteration in PIK3CA in a sample from an individual having an ILC metastasis, the individual is classified as a candidate to receive a treatment comprising a kinase inhibitor, an AKT inhibitor, an EZH2 inhibitor, or an mTOR inhibitor. In some embodiments, responsive to knowledge of the presence of an alteration in PIK3CA in a sample from an individual having an ILC metastasis, the individual is classified or identified as likely to respond to a treatment comprising a kinase inhibitor, an AKT inhibitor, an EZH2 inhibitor, or an mTOR inhibitor. In some embodiments, the alteration comprises one or more of a substitution of one or more nucleotides, an insertion of one or more nucleotides, or a deletion of one or more nucleotides in PIK3CA. In some embodiments, the methods further comprise generating a report, e.g., as described herein. In some embodiments, the report comprises one or more treatment options identified or selected, e.g., based, at least in part, on the detection of the alteration in PIK3CA in a sample from the individual. In some embodiments, the report comprises one or more treatment options identified or selected, e.g., based, at least in part, on the presence of the alteration in PIK3CA in a sample from the individual. In some embodiments, the report comprises one or more treatment options identified or selected, e.g., based, at least in part, on acquiring knowledge of the presence of the alteration in PIK3CA in a sample from the individual. In some embodiments, the one or more treatment options include an anti-cancer therapy provided herein, e.g., a kinase inhibitor, an AKT inhibitor, an EZH2 inhibitor, or an mTOR inhibitor.

In some embodiments, the presence of an alteration in BRCA1 in a sample from an individual having an ILC metastasis identifies the individual as one who may benefit from a treatment comprising a PARP inhibitor, a Chk1/2 inhibitor, or a Wee1 inhibitor. In some embodiments, responsive to knowledge of the presence of an alteration in BRCA1 in a sample from an individual having an ILC metastasis, the individual is classified as a candidate to receive a treatment comprising a PARP inhibitor, a Chk1/2 inhibitor, or a Wee1 inhibitor. In some embodiments, responsive to knowledge of the presence of an alteration in BRCA1 in a sample from an individual having an ILC metastasis, the individual is classified or identified as likely to respond to a treatment comprising a PARP inhibitor, a Chk1/2 inhibitor, or a Wee1 inhibitor. In some embodiments, the methods further comprise generating a report, e.g., as described herein. In some embodiments, the report comprises one or more treatment options identified or selected, e.g., based, at least in part, on the detection of the alteration in BRCA1 in a sample from the individual. In some embodiments, the report comprises one or more treatment options identified or selected, e.g., based, at least in part, on the presence of the alteration in BRCA1 in a sample from the individual. In some embodiments, the report comprises one or more treatment options identified or selected, e.g., based, at least in part, on acquiring knowledge of the presence of the alteration in BRCA1 in a sample from the individual. In some embodiments, the one or more treatment options include an anti-cancer therapy provided herein, e.g., a PARP inhibitor, a Chk1/2 inhibitor, or a Wee1 inhibitor.

In some embodiments, the presence of an alteration in BRCA2 in a sample from an individual having an ILC metastasis identifies the individual as one who may benefit from a treatment comprising a PARP inhibitor, a Chk1/2 inhibitor, or a Wee1 inhibitor. In some embodiments, responsive to knowledge of the presence of an alteration in BRCA2 in a sample from an individual having an ILC metastasis, the individual is classified as a candidate to receive a treatment comprising a PARP inhibitor, a Chk1/2 inhibitor, or a Wee1 inhibitor. In some embodiments, responsive to knowledge of the presence of an alteration in BRCA2 in a sample from an individual having an ILC metastasis, the individual is classified or identified as likely to respond to a treatment comprising a PARP inhibitor, a Chk1/2 inhibitor, or a Wee1 inhibitor. In some embodiments, the methods further comprise generating a report, e.g., as described herein. In some embodiments, the report comprises one or more treatment options identified or selected, e.g., based, at least in part, on the detection of the alteration in BRCA2 in a sample from the individual. In some embodiments, the report comprises one or more treatment options identified or selected, e.g., based, at least in part, on the presence of the alteration in BRCA2 in a sample from the individual. In some embodiments, the report comprises one or more treatment options identified or selected, e.g., based, at least in part, on acquiring knowledge of the presence of the alteration in BRCA2 in a sample from the individual. In some embodiments, the one or more treatment options include an anti-cancer therapy provided herein, e.g., a PARP inhibitor, a Chk1/2 inhibitor, or a Wee1 inhibitor.

In some embodiments, the presence of an alteration in BRCA1 and BRCA2 in a sample from an individual having an ILC metastasis identifies the individual as one who may benefit from a treatment comprising a PARP inhibitor, a Chk1/2 inhibitor, or a Wee1 inhibitor. In some embodiments, responsive to knowledge of the presence of an alteration in BRCA1 and BRCA2 in a sample from an individual having an ILC metastasis, the individual is classified as a candidate to receive a treatment comprising a PARP inhibitor, a Chk1/2 inhibitor, or a Wee1 inhibitor. In some embodiments, responsive to knowledge of the presence of an alteration in BRCA1 and BRCA2 in a sample from an individual having an ILC metastasis, the individual is classified or identified as likely to respond to a treatment comprising a PARP inhibitor, a Chk1/2 inhibitor, or a Wee1 inhibitor. In some embodiments, the methods further comprise generating a report, e.g., as described herein. In some embodiments, the report comprises one or more treatment options identified or selected, e.g., based, at least in part, on the detection of the alteration in BRCA1 and BRCA2 in a sample from the individual. In some embodiments, the report comprises one or more treatment options identified or selected, e.g., based, at least in part, on the presence of the alteration in BRCA1 and BRCA2 in a sample from the individual. In some embodiments, the report comprises one or more treatment options identified or selected, e.g., based, at least in part, on acquiring knowledge of the presence of the alteration in BRCA1 and BRCA2 in a sample from the individual. In some embodiments, the one or more treatment options include an anti-cancer therapy provided herein, e.g., a PARP inhibitor, a Chk1/2 inhibitor, or a Wee1 inhibitor.

In some embodiments, the presence of an alteration in ESR1 in a sample from an individual having an ILC metastasis identifies the individual as one who may benefit from a treatment comprising a Selective Estrogen Receptor Modulator (SERM), an anti-estrogen, or an aromatase inhibitor. In some embodiments, responsive to knowledge of the presence of an alteration in ESR1 in a sample from an individual having an ILC metastasis, the individual is classified as a candidate to receive a treatment comprising a Selective Estrogen Receptor Modulator (SERM), an anti-estrogen, or an aromatase inhibitor. In some embodiments, responsive to knowledge of the presence of an alteration in ESR1 in a sample from an individual having an ILC metastasis, the individual is classified or identified as likely to respond to a treatment comprising a Selective Estrogen Receptor Modulator (SERM), an anti-estrogen, or an aromatase inhibitor. In some embodiments, the alteration comprises one or more of a substitution of one or more nucleotides, an insertion of one or more nucleotides, or a deletion of one or more nucleotides in ESR1. In some embodiments, the methods further comprise generating a report, e.g., as described herein. In some embodiments, the report comprises one or more treatment options identified or selected, e.g., based, at least in part, on the detection of the alteration in ESR1 in a sample from the individual. In some embodiments, the report comprises one or more treatment options identified or selected, e.g., based, at least in part, on the presence of the alteration in ESR1 in a sample from the individual. In some embodiments, the report comprises one or more treatment options identified or selected, e.g., based, at least in part, on acquiring knowledge of the presence of the alteration in ESR1 in a sample from the individual. In some embodiments, the one or more treatment options include an anti-cancer therapy provided herein, e.g., a Selective Estrogen Receptor Modulator (SERM), an anti-estrogen, or an aromatase inhibitor.

In some embodiments, the presence of an alteration in NF1 in a sample from an individual having an ILC metastasis identifies the individual as one who may benefit from a treatment comprising a kinase inhibitor, an mTOR inhibitor, an EGFR inhibitor, a glutaminase inhibitor, or a MEK inhibitor. In some embodiments, responsive to knowledge of the presence of an alteration in NF1 in a sample from an individual having an ILC metastasis, the individual is classified as a candidate to receive a treatment comprising a kinase inhibitor, an mTOR inhibitor, an EGFR inhibitor, a glutaminase inhibitor, or a MEK inhibitor. In some embodiments, responsive to knowledge of the presence of an alteration in NF1 in a sample from an individual having an ILC metastasis, the individual is classified or identified as likely to respond to a treatment comprising a kinase inhibitor, an mTOR inhibitor, an EGFR inhibitor, a glutaminase inhibitor, or a MEK inhibitor. In some embodiments, the alteration comprises one or more of a substitution of one or more nucleotides, an insertion of one or more nucleotides, or a deletion of one or more nucleotides in NF1. In some embodiments, the methods further comprise generating a report, e.g., as described herein. In some embodiments, the report comprises one or more treatment options identified or selected, e.g., based, at least in part, on the detection of the alteration in NF1 in a sample from the individual. In some embodiments, the report comprises one or more treatment options identified or selected, e.g., based, at least in part, on the presence of the alteration in NF1 in a sample from the individual. In some embodiments, the report comprises one or more treatment options identified or selected, e.g., based, at least in part, on acquiring knowledge of the presence of the alteration in NF1 in a sample from the individual. In some embodiments, the one or more treatment options include an anti-cancer therapy provided herein, e.g., a kinase inhibitor, an mTOR inhibitor, an EGFR inhibitor, a glutaminase inhibitor, or a MEK inhibitor.

In some embodiments, the presence of an alteration in RB1 in a sample from an individual having an ILC metastasis identifies the individual as one who may benefit from a treatment comprising a SOX2 inhibitor, an EZH2 inhibitor, a chemotherapy, a checkpoint kinase (CHK) inhibitor, a CDC25 phosphatase inhibitor, a polo-like kinase (PLK) inhibitor, or an aurora kinase (AURK) inhibitor. In some embodiments, responsive to knowledge of the presence of an alteration in RB1 in a sample from an individual having an ILC metastasis, the individual is classified as a candidate to receive a treatment comprising a SOX2 inhibitor, an EZH2 inhibitor, a chemotherapy, a checkpoint kinase (CHK) inhibitor, a CDC25 phosphatase inhibitor, a polo-like kinase (PLK) inhibitor, or an aurora kinase (AURK) inhibitor. In some embodiments, responsive to knowledge of the presence of an alteration in RB1 in a sample from an individual having an ILC metastasis, the individual is classified or identified as likely to respond to a treatment comprising a SOX2 inhibitor, an EZH2 inhibitor, a chemotherapy, a checkpoint kinase (CHK) inhibitor, a CDC25 phosphatase inhibitor, a polo-like kinase (PLK) inhibitor, or an aurora kinase (AURK) inhibitor. In some embodiments, the alteration comprises one or more of a substitution of one or more nucleotides, an insertion of one or more nucleotides, or a deletion of one or more nucleotides in RB1. In some embodiments, the methods further comprise generating a report, e.g., as described herein. In some embodiments, the report comprises one or more treatment options identified or selected, e.g., based, at least in part, on the detection of the alteration in RB1 in a sample from the individual. In some embodiments, the report comprises one or more treatment options identified or selected, e.g., based, at least in part, on the presence of the alteration in RB1 in a sample from the individual. In some embodiments, the report comprises one or more treatment options identified or selected, e.g., based, at least in part, on acquiring knowledge of the presence of the alteration in RB1 in a sample from the individual. In some embodiments, the one or more treatment options include an anti-cancer therapy provided herein, e.g., a SOX2 inhibitor, an EZH2 inhibitor, a chemotherapy, a checkpoint kinase (CHK) inhibitor, a CDC25 phosphatase inhibitor, a polo-like kinase (PLK) inhibitor, or an aurora kinase (AURK) inhibitor.

In some embodiments, the presence of an alteration in KRAS in a sample from an individual having an ILC metastasis identifies the individual as one who may benefit from a treatment comprising a kinase inhibitor, a farnesyltransferase inhibitor, a geranylgeranyltransferase inhibitor, a palmitoylation inhibitor, an inhibitor of methylation cleavage, a Raf inhibitor, a MEK inhibitor, an mTOR inhibitor, or an agent that inhibits the modification or post-translational processing of KRAS. In some embodiments, responsive to knowledge of the presence of an alteration in KRAS in a sample from an individual having an ILC metastasis, the individual is classified as a candidate to receive a treatment comprising a kinase inhibitor, a farnesyltransferase inhibitor, a geranylgeranyltransferase inhibitor, a palmitoylation inhibitor, an inhibitor of methylation cleavage, a Raf inhibitor, a MEK inhibitor, an mTOR inhibitor, or an agent that inhibits the modification or post-translational processing of KRAS. In some embodiments, responsive to knowledge of the presence of an alteration in KRAS in a sample from an individual having an ILC metastasis, the individual is classified or identified as likely to respond to a treatment comprising a kinase inhibitor, a farnesyltransferase inhibitor, a geranylgeranyltransferase inhibitor, a palmitoylation inhibitor, an inhibitor of methylation cleavage, a Raf inhibitor, a MEK inhibitor, an mTOR inhibitor, or an agent that inhibits the modification or post-translational processing of KRAS. In some embodiments, the methods further comprise generating a report, e.g., as described herein. In some embodiments, the report comprises one or more treatment options identified or selected, e.g., based, at least in part, on the detection of the alteration in KRAS in a sample from the individual. In some embodiments, the report comprises one or more treatment options identified or selected, e.g., based, at least in part, on the presence of the alteration in KRAS in a sample from the individual. In some embodiments, the report comprises one or more treatment options identified or selected, e.g., based, at least in part, on acquiring knowledge of the presence of the alteration in KRAS in a sample from the individual. In some embodiments, the one or more treatment options include an anti-cancer therapy provided herein, e.g., a kinase inhibitor, a farnesyltransferase inhibitor, a geranylgeranyltransferase inhibitor, a palmitoylation inhibitor, an inhibitor of methylation cleavage, a Raf inhibitor, a MEK inhibitor, an mTOR inhibitor, or an agent that inhibits the modification or post-translational processing of KRAS.

In some embodiments, the presence of an alteration in ERBB2 in a sample from an individual having an ILC metastasis identifies the individual as one who may benefit from a treatment comprising a kinase inhibitor, a small molecule, an antibody or antibody fragment, a cellular immunotherapy, or a pan-ERBB inhibitor. In some embodiments, responsive to knowledge of the presence of an alteration in ERBB2 in a sample from an individual having an ILC metastasis, the individual is classified as a candidate to receive a treatment comprising a kinase inhibitor, a small molecule, an antibody or antibody fragment, a cellular immunotherapy, or a pan-ERBB inhibitor. In some embodiments, responsive to knowledge of the presence of an alteration in ERBB2 in a sample from an individual having an ILC metastasis, the individual is classified or identified as likely to respond to a treatment comprising a kinase inhibitor, a small molecule, an antibody or antibody fragment, a cellular immunotherapy, or a pan-ERBB inhibitor. In some embodiments, the methods further comprise generating a report, e.g., as described herein. In some embodiments, the report comprises one or more treatment options identified or selected, e.g., based, at least in part, on the detection of the alteration in ERBB2 in a sample from the individual. In some embodiments, the report comprises one or more treatment options identified or selected, e.g., based, at least in part, on the presence of the alteration in ERBB2 in a sample from the individual. In some embodiments, the report comprises one or more treatment options identified or selected, e.g., based, at least in part, on acquiring knowledge of the presence of the alteration in ERBB2 in a sample from the individual. In some embodiments, the one or more treatment options include an anti-cancer therapy provided herein, e.g., a kinase inhibitor, a small molecule, an antibody or antibody fragment, a cellular immunotherapy, or a pan-ERBB inhibitor. In some embodiments, the kinase inhibitor is a multi-specific kinase inhibitor, a reversible HER2 inhibitor, an irreversible HER2 inhibitor, a pan-ERBB inhibitor, a dual HER2 inhibitor, a HER2-specific inhibitor, an EGFR inhibitor, or a dual EGFR/ERBB inhibitor.

In some embodiments, the presence of an alteration in BRAF in a sample from an individual having an ILC metastasis identifies the individual as one who may benefit from a treatment comprising a kinase inhibitor. In some embodiments, responsive to knowledge of the presence of an alteration in BRAF in a sample from an individual having an ILC metastasis, the individual is classified as a candidate to receive a treatment comprising a kinase inhibitor. In some embodiments, responsive to knowledge of the presence of an alteration in BRAF in a sample from an individual having an ILC metastasis, the individual is classified or identified as likely to respond to a treatment comprising a kinase inhibitor. In some embodiments, the methods further comprise generating a report, e.g., as described herein. In some embodiments, the report comprises one or more treatment options identified or selected, e.g., based, at least in part, on the detection of the alteration in BRAF in a sample from the individual. In some embodiments, the report comprises one or more treatment options identified or selected, e.g., based, at least in part, on the presence of the alteration in BRAF in a sample from the individual. In some embodiments, the report comprises one or more treatment options identified or selected, e.g., based, at least in part, on acquiring knowledge of the presence of the alteration in BRAF in a sample from the individual. In some embodiments, the one or more treatment options include an anti-cancer therapy provided herein, e.g., a kinase inhibitor.

In some embodiments, the presence of an alteration in ARID1A in a sample from an individual having an ILC metastasis identifies the individual as one who may benefit from a treatment comprising a PARP inhibitor, a bromodomain-containing protein 4 (BRD4) inhibitor, an HDAC inhibitor, a PI3K inhibitor, an ATR inhibitor, an EZH2 inhibitor, an AKT inhibitor, a TrxR inhibitor, a GSH inhibitor, or an immune checkpoint inhibitor. In some embodiments, responsive to knowledge of the presence of an alteration in ARID1A in a sample from an individual having an ILC metastasis, the individual is classified as a candidate to receive a treatment comprising a PARP inhibitor, a bromodomain-containing protein 4 (BRD4) inhibitor, an HDAC inhibitor, a PI3K inhibitor, an ATR inhibitor, an EZH2 inhibitor, an AKT inhibitor, a TrxR inhibitor, a GSH inhibitor, or an immune checkpoint inhibitor. In some embodiments, responsive to knowledge of the presence of an alteration in ARID1A in a sample from an individual having an ILC metastasis, the individual is classified or identified as likely to respond to a treatment comprising a PARP inhibitor, a bromodomain-containing protein 4 (BRD4) inhibitor, an HDAC inhibitor, a PI3K inhibitor, an ATR inhibitor, an EZH2 inhibitor, an AKT inhibitor, a TrxR inhibitor, a GSH inhibitor, or an immune checkpoint inhibitor. In some embodiments, the alteration comprises one or more of a substitution of one or more nucleotides, an insertion of one or more nucleotides, or a deletion of one or more nucleotides in ARID1A. In some embodiments, the methods further comprise generating a report, e.g., as described herein. In some embodiments, the report comprises one or more treatment options identified or selected, e.g., based, at least in part, on the detection of the alteration in ARID1A in a sample from the individual. In some embodiments, the report comprises one or more treatment options identified or selected, e.g., based, at least in part, on the presence of the alteration in ARID1A in a sample from the individual. In some embodiments, the report comprises one or more treatment options identified or selected, e.g., based, at least in part, on acquiring knowledge of the presence of the alteration in ARID1A in a sample from the individual. In some embodiments, the one or more treatment options include an anti-cancer therapy provided herein, e.g., a PARP inhibitor, a bromodomain-containing protein 4 (BRD4) inhibitor, an HDAC inhibitor, a PI3K inhibitor, an ATR inhibitor, an EZH2 inhibitor, an AKT inhibitor, a TrxR inhibitor, a GSH inhibitor, or an immune checkpoint inhibitor.

In some embodiments, the presence of an alteration in PTEN in a sample from an individual having an ILC metastasis identifies the individual as one who may benefit from a treatment comprising a PI3K inhibitor, an AKT inhibitor, an mTOR inhibitor, or a MET inhibitor. In some embodiments, responsive to knowledge of the presence of an alteration in PTEN in a sample from an individual having an ILC metastasis, the individual is classified as a candidate to receive a treatment comprising a PI3K inhibitor, an AKT inhibitor, an mTOR inhibitor, or a MET inhibitor. In some embodiments, responsive to knowledge of the presence of an alteration in PTEN in a sample from an individual having an ILC metastasis, the individual is classified or identified as likely to respond to a treatment comprising a PI3K inhibitor, an AKT inhibitor, an mTOR inhibitor, or a MET inhibitor. In some embodiments, the alteration is a PTEN deletion. In some embodiments, the methods further comprise generating a report, e.g., as described herein. In some embodiments, the report comprises one or more treatment options identified or selected, e.g., based, at least in part, on the detection of the alteration in PTEN in a sample from the individual. In some embodiments, the report comprises one or more treatment options identified or selected, e.g., based, at least in part, on the presence of the alteration in PTEN in a sample from the individual. In some embodiments, the report comprises one or more treatment options identified or selected, e.g., based, at least in part, on acquiring knowledge of the presence of the alteration in PTEN in a sample from the individual. In some embodiments, the one or more treatment options include an anti-cancer therapy provided herein, e.g., a PI3K inhibitor, an AKT inhibitor, an mTOR inhibitor, or a MET inhibitor.

In some embodiments, the presence of an alteration in FGFR2 in a sample from an individual having an ILC metastasis identifies the individual as one who may benefit from a treatment comprising a kinase inhibitor. In some embodiments, responsive to knowledge of the presence of an alteration in FGFR2 in a sample from an individual having an ILC metastasis, the individual is classified as a candidate to receive a treatment comprising a kinase inhibitor. In some embodiments, responsive to knowledge of the presence of an alteration in FGFR2 in a sample from an individual having an ILC metastasis, the individual is classified or identified as likely to respond to a treatment comprising a kinase inhibitor. In some embodiments, the methods further comprise generating a report, e.g., as described herein. In some embodiments, the report comprises one or more treatment options identified or selected, e.g., based, at least in part, on the detection of the alteration in FGFR2 in a sample from the individual. In some embodiments, the report comprises one or more treatment options identified or selected, e.g., based, at least in part, on the presence of the alteration in FGFR2 in a sample from the individual. In some embodiments, the report comprises one or more treatment options identified or selected, e.g., based, at least in part, on acquiring knowledge of the presence of the alteration in FGFR2 in a sample from the individual. In some embodiments, the one or more treatment options include an anti-cancer therapy provided herein, e.g., a kinase inhibitor.

In some embodiments, the presence of an alteration in SMAD4 in a sample from an individual having an ILC metastasis identifies the individual as one who may benefit from a treatment comprising a PARP inhibitor. In some embodiments, responsive to knowledge of the presence of an alteration in SMAD4 in a sample from an individual having an ILC metastasis, the individual is classified as a candidate to receive a treatment comprising a PARP inhibitor. In some embodiments, responsive to knowledge of the presence of an alteration in SMAD4 in a sample from an individual having an ILC metastasis, the individual is classified or identified as likely to respond to a treatment comprising a PARP inhibitor. In some embodiments, the methods further comprise generating a report, e.g., as described herein. In some embodiments, the report comprises one or more treatment options identified or selected, e.g., based, at least in part, on the detection of the alteration in SMAD4 in a sample from the individual. In some embodiments, the report comprises one or more treatment options identified or selected, e.g., based, at least in part, on the presence of the alteration in SMAD4 in a sample from the individual. In some embodiments, the report comprises one or more treatment options identified or selected, e.g., based, at least in part, on acquiring knowledge of the presence of the alteration in SMAD4 in a sample from the individual. In some embodiments, the one or more treatment options include an anti-cancer therapy provided herein, e.g., a PARP inhibitor.

In some embodiments, the presence of an alteration in PTPN11 in a sample from an individual having an ILC metastasis identifies the individual as one who may benefit from a treatment comprising a small molecule inhibitor or a kinase inhibitor. In some embodiments, responsive to knowledge of the presence of an alteration in PTPN11 in a sample from an individual having an ILC metastasis, the individual is classified as a candidate to receive a treatment comprising a small molecule inhibitor or a kinase inhibitor. In some embodiments, responsive to knowledge of the presence of an alteration in PTPN11 in a sample from an individual having an ILC metastasis, the individual is classified or identified as likely to respond to a treatment comprising a small molecule inhibitor or a kinase inhibitor. In some embodiments, the alteration comprises one or more of a substitution of one or more nucleotides, an insertion of one or more nucleotides, or a deletion of one or more nucleotides in PTPN11. In some embodiments, the methods further comprise generating a report, e.g., as described herein. In some embodiments, the report comprises one or more treatment options identified or selected, e.g., based, at least in part, on the detection of the alteration in PTPN11 in a sample from the individual. In some embodiments, the report comprises one or more treatment options identified or selected, e.g., based, at least in part, on the presence of the alteration in PTPN11 in a sample from the individual. In some embodiments, the report comprises one or more treatment options identified or selected, e.g., based, at least in part, on acquiring knowledge of the presence of the alteration in PTPN11 in a sample from the individual. In some embodiments, the one or more treatment options include an anti-cancer therapy provided herein, e.g., a small molecule inhibitor or a kinase inhibitor.

In some embodiments, the presence of an alteration in TERT in a sample from an individual having an ILC metastasis identifies the individual as one who may benefit from a treatment comprising a small molecule inhibitor, a modified nucleotide or nucleoside analog, a G-quadruplex stabilizer, a heat shock protein (HSP) inhibitor, or a MYC inhibitor. In some embodiments, responsive to knowledge of the presence of an alteration in TERT in a sample from an individual having an ILC metastasis, the individual is classified as a candidate to receive a treatment comprising a small molecule inhibitor, a modified nucleotide or nucleoside analog, a G-quadruplex stabilizer, a heat shock protein (HSP) inhibitor, or a MYC inhibitor. In some embodiments, responsive to knowledge of the presence of an alteration in TERT in a sample from an individual having an ILC metastasis, the individual is classified or identified as likely to respond to a treatment comprising a small molecule inhibitor, a modified nucleotide or nucleoside analog, a G-quadruplex stabilizer, a heat shock protein (HSP) inhibitor, or a MYC inhibitor. In some embodiments, the alteration comprises one or more of a substitution of one or more nucleotides, an insertion of one or more nucleotides, or a deletion of one or more nucleotides in TERT. In some embodiments, the methods further comprise generating a report, e.g., as described herein. In some embodiments, the report comprises one or more treatment options identified or selected, e.g., based, at least in part, on the detection of the alteration in TERT in a sample from the individual. In some embodiments, the report comprises one or more treatment options identified or selected, e.g., based, at least in part, on the presence of the alteration in TERT in a sample from the individual. In some embodiments, the report comprises one or more treatment options identified or selected, e.g., based, at least in part, on acquiring knowledge of the presence of the alteration in TERT in a sample from the individual. In some embodiments, the one or more treatment options include an anti-cancer therapy provided herein, e.g., a small molecule inhibitor, a modified nucleotide or nucleoside analog, a G-quadruplex stabilizer, a heat shock protein (HSP) inhibitor, or a MYC inhibitor.

In some embodiments, the presence of an alteration in ALK in a sample from an individual having an ILC metastasis identifies the individual as one who may benefit from a treatment comprising a kinase inhibitor, a heat shock protein (HSP) inhibitor or a MYC inhibitor. In some embodiments, responsive to knowledge of the presence of an alteration in ALK in a sample from an individual having an ILC metastasis, the individual is classified as a candidate to receive a treatment comprising a kinase inhibitor, a heat shock protein (HSP) inhibitor or a MYC inhibitor. In some embodiments, responsive to knowledge of the presence of an alteration in ALK in a sample from an individual having an ILC metastasis, the individual is classified or identified as likely to respond to a treatment comprising a kinase inhibitor, a heat shock protein (HSP) inhibitor or a MYC inhibitor. In some embodiments, the alteration comprises one or more of a substitution of one or more nucleotides, an insertion of one or more nucleotides, or a deletion of one or more nucleotides in ALK. In some embodiments, the methods further comprise generating a report, e.g., as described herein. In some embodiments, the report comprises one or more treatment options identified or selected, e.g., based, at least in part, on the detection of the alteration in ALK in a sample from the individual. In some embodiments, the report comprises one or more treatment options identified or selected, e.g., based, at least in part, on the presence of the alteration in ALK in a sample from the individual. In some embodiments, the report comprises one or more treatment options identified or selected, e.g., based, at least in part, on acquiring knowledge of the presence of the alteration in ALK in a sample from the individual. In some embodiments, the one or more treatment options include an anti-cancer therapy provided herein, e.g., a kinase inhibitor, a heat shock protein (HSP) inhibitor or a MYC inhibitor.

In some embodiments, the presence of an alteration in APC in a sample from an individual having an ILC metastasis identifies the individual as one who may benefit from a treatment comprising a beta-catenin inhibitor or an APC inhibitor. In some embodiments, the alteration comprises one or more of a substitution of one or more nucleotides, an insertion of one or more nucleotides, or a deletion of one or more nucleotides in APC. In some embodiments, responsive to knowledge of the presence of an alteration in APC in a sample from an individual having an ILC metastasis, the individual is classified as a candidate to receive a treatment comprising a beta-catenin inhibitor or an APC inhibitor. In some embodiments, responsive to knowledge of the presence of an alteration in APC in a sample from an individual having an ILC metastasis, the individual is classified or identified as likely to respond to a treatment comprising a beta-catenin inhibitor or an APC inhibitor. In some embodiments, the methods further comprise generating a report, e.g., as described herein. In some embodiments, the report comprises one or more treatment options identified or selected, e.g., based, at least in part, on the detection of the alteration in APC in a sample from the individual. In some embodiments, the report comprises one or more treatment options identified or selected, e.g., based, at least in part, on the presence of the alteration in APC in a sample from the individual. In some embodiments, the report comprises one or more treatment options identified or selected, e.g., based, at least in part, on acquiring knowledge of the presence of the alteration in APC in a sample from the individual. In some embodiments, the one or more treatment options include an anti-cancer therapy provided herein, e.g., a beta-catenin inhibitor or an APC inhibitor.

Reporting

In certain aspects, provided herein are methods that include generating and/or providing a report about an ILC metastasis of the disclosure, or about the presence of high tumor mutational burden (e.g., of at least about 10 mut/Mb), PD-L1 positivity or expression, or an alteration in one or more genes (e.g., PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D) in sample from an individual.

In some embodiments, a report according to the present disclosure comprises one or more of the following, e.g., information about the presence or absence of high tumor mutational burden (e.g., of at least about 10 mut/Mb), PD-L1 positivity or expression, or an alteration in one or more genes (e.g., PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D) in a sample obtained from an individual, such as an individual having an ILC metastasis. In one embodiment, a report according to the present disclosure indicates that one or more of the following is present in a sample obtained from an individual (e.g., an individual having an ILC metastasis): high tumor mutational burden (e.g., of at least about 10 mut/Mb), PD-L1 positivity or expression, or an alteration in one or more genes (e.g., PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D). In one embodiment, a report according to the present disclosure indicates that one or more of the following is not present in a sample obtained from an individual (e.g., an individual having an ILC metastasis): a high tumor mutational burden (e.g., of at least about 10 mut/Mb), PD-L1 positivity or expression, or an alteration in one or more genes (e.g., PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D). In one embodiment, a report according to the present disclosure indicates that one or more of the following has been detected in a sample obtained from an individual (e.g., an individual having an ILC metastasis): high tumor mutational burden (e.g., of at least about 10 mut/Mb), PD-L1 positivity or expression, or an alteration in one or more genes (e.g., PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D). In one embodiment, a report according to the present disclosure indicates that one or more of the following has not been detected in a sample obtained from an individual (e.g., an individual having an ILC metastasis): a high tumor mutational burden (e.g., of at least about 10 mut/Mb), PD-L1 positivity or expression, or an alteration in one or more genes (e.g., PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D). In some embodiments, the report comprises an identifier for the individual or patient from which the sample was obtained.

In some embodiments, the report also includes information on the role of one or more of the following in disease, such as in cancer (e.g., ILC metastasis): high tumor mutational burden (e.g., of at least about 10 mut/Mb), PD-L1 positivity or expression, or an alteration in one or more genes (e.g., PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D). Such information can include, for example, information on prognosis, resistance, potential or suggested therapeutic options, e.g., such as a treatment selected or identified according to the methods provided herein, and/or therapeutic options that should be avoided. The report can include information on the likely effectiveness, acceptability, and/or the advisability of applying the therapeutic option (e.g., such as a treatment selected or identified according to the methods provided herein) to an individual, e.g., an individual having one or more of a high tumor mutational burden (e.g., of at least about 10 mut/Mb), PD-L1 positivity or expression, or an alteration in one or more genes (e.g., PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D). In some embodiments, the report can include information, or a recommendation on, the administration of a drug (e.g., such as a treatment selected or identified according to the methods provided herein), e.g., the administration at a dosage or in a treatment regimen to the patient, e.g., in combination with other treatments (e.g., a second therapeutic agent described herein). In some embodiments, the report comprises information or a recommendation for at least one, at least two, at least three, at least four, at least five, at least six, at least seven, at least eight, at least nine, at least ten, or more treatments (e.g., such as a treatment selected or identified according to the methods provided herein and/or a second therapeutic agent described herein).

Also provided herein are methods of generating a report according to the present disclosure. In some embodiments, a report according to the present disclosure is generated by a method comprising one or more of the steps of obtaining a sample, such as a tumor sample, from an individual (e.g., an individual or a patient with an ILC metastasis); detecting one or more of a high tumor mutational burden (e.g., of at least about 10 mut/Mb), PD-L1 positivity or expression, or an alteration in one or more genes (e.g., PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D) in the sample, or acquiring knowledge of the presence of one or more of a high tumor mutational burden (e.g., of at least about 10 mut/Mb), PD-L1 positivity or expression, or an alteration in one or more genes (e.g., PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D) in the sample; and generating a report, wherein the report comprises one or more of: information about the presence or absence of a high tumor mutational burden (e.g., of at least about 10 mut/Mb), PD-L1 positivity or expression, or an alteration in one or more genes (e.g., PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D) in the sample; an identifier for the individual from which the sample was obtained; information on the role of a high tumor mutational burden (e.g., of at least about 10 mut/Mb), PD-L1 positivity or expression, or an alteration in one or more genes (e.g., PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D) in disease (e.g., such as in cancer, e.g., an ILC metastasis); information on prognosis, resistance, or potential or suggested therapeutic options, e.g., such as a treatment selected or identified according to the methods provided herein; information on the likely effectiveness, acceptability, or the advisability of applying a therapeutic option (e.g., such as a treatment selected or identified according to the methods provided herein) to the individual; a recommendation or information on the administration of a drug (e.g., such as a treatment selected or identified according to the methods provided herein); or a recommendation or information on the dosage or treatment regimen of a drug (e.g., such as a treatment selected or identified according to the methods provided herein), e.g., in combination with other treatments (e.g., a second therapeutic agent described herein). In some embodiments, the report generated is a personalized cancer report.

A report according to the present disclosure may be in an electronic, web-based, or paper form. The report may be provided to an individual or a patient (e.g., an individual or a patient with an ILC metastasis described herein), or an individual or entity other than the individual or patient (e.g., other than the individual or a patient with an ILC metastasis described herein), such as a caregiver, a physician, an oncologist, a hospital, a clinic, a third party payor, an insurance company, or a government entity. In some embodiments, the report is provided or delivered to an entity, e.g., an entity described herein, within any of about 1 day or more, about 7 days or more, about 14 days or more, about 21 days or more, about 30 days or more, about 45 days or more, about 60 days or more, from obtaining a sample from an individual (e.g., an individual or a patient with an ILC metastasis described herein). In some embodiments, the report is provided or delivered to an entity, e.g., an entity described herein, within any of about 1 day or more, about 7 days or more, about 14 days or more, about 21 days or more, about 30 days or more, about 45 days or more, about 60 days or more, from detecting one or more of a high tumor mutational burden (e.g., of at least about 10 mut/Mb), PD-L1 positivity or expression, or an alteration in one or more genes (e.g., PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D) in a sample obtained from an individual (e.g., an individual having an ILC metastasis). In some embodiments, the report is provided or delivered to an entity, e.g., an entity described herein, within any of about 1 day or more, about 7 days or more, about 14 days or more, about 21 days or more, about 30 days or more, about 45 days or more, about 60 days or more, from acquiring knowledge of or detecting the presence of one or more of a high tumor mutational burden (e.g., of at least about 10 mut/Mb), PD-L1 positivity or expression, or an alteration in one or more genes (e.g., PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D) in a sample obtained from an individual (e.g., an individual having an ILC metastasis).

The method steps of the methods described herein are intended to include any suitable method of causing one or more other parties or entities to perform the steps, unless a different meaning is expressly provided or otherwise clear from the context. Such parties or entities need not be under the direction or control of any other party or entity, and need not be located within a particular jurisdiction. Thus for example, a description or recitation of “adding a first number to a second number” includes causing one or more parties or entities to add the two numbers together. For example, if person X engages in an arm's length transaction with person Y to add the two numbers, and person Y indeed adds the two numbers, then both persons X and Y perform the step as recited: person Y by virtue of the fact that he actually added the numbers, and person X by virtue of the fact that he caused person Y to add the numbers. Furthermore, if person X is located within the United States and person Y is located outside the United States, then the method is performed in the United States by virtue of person X's participation in causing the step to be performed.

### Anti-Cancer Agents

In some embodiments, the methods of the present disclosure comprise administering an anti-cancer therapy to an individual. In some embodiments, the anti-cancer therapy comprises an anti-cancer agent. In some embodiments, the anti-cancer agent is a small molecule inhibitor, a chemotherapy, an antibody or antibody fragment, or a nucleic acid. In some embodiments, the anti-cancer agent is administered in combination with one or more additional anti-cancer therapies. In some embodiments, the anti-cancer agent is administered in response to knowledge of or detection of one or more biomarkers, e.g., one or more biomarkers provided herein, in a sample from an individual having an ILC metastasis.

In some embodiments, the methods provided herein comprise administering to an individual an effective amount of an immune checkpoint therapy, e.g., a checkpoint inhibitor. As is known in the art, a checkpoint inhibitor targets at least one immune checkpoint protein to alter the regulation of an immune response. Immune checkpoint proteins include, e.g., CTLA4, PD-L1, PD-1, PD-L2, VISTA, B7-H2, B7-H3, B7-H4, B7-H6, 2B4, ICOS, HVEM, CEACAM, LAIR1, CD80, CD86, CD276, VTCN1, MHC class I, MHC class II, GALS, adenosine, TGFR, CSF1R, MICA/B, arginase, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, LAG-3, BTLA, IDO, OX40, and A2aR. In some embodiments, molecules involved in regulating immune checkpoints include, but are not limited to: PD-1 (CD279), PD-L1 (B7-H1, CD274), PD-L2 (B7-CD, CD273), CTLA-4 (CD152), HVEM, BTLA (CD272), a killer-cell immunoglobulin-like receptor (KIR), LAG-3 (CD223), TIM-3 (HAVCR2), CEACAM, CEACAM-1, CEACAM-3, CEACAM-5, GALS, VISTA (PD-1H), TIGIT, LAIR1, CD160, 2B4, TGFRbeta, A2AR, GITR (CD357), CD80 (B7-1), CD86 (B7-2), CD276 (B7-H3), VTCNI (B7-H4), MHC class I, MHC class II, GALS, adenosine, TGFR, B7-H1, OX40 (CD134), CD94 (KLRD1), CD137 (4-1BB), CD137L (4-1BBL), CD40, IDO, CSF1R, CD40L, CD47, CD70 (CD27L), CD226, HHLA2, ICOS (CD278), ICOSL (CD275), LIGHT (TNFSF14, CD258), NKG2a, NKG2d, OX40L (CD134L), PVR (NECL5, CD155), SIRPa, MICA/B, and/or arginase. In some embodiments, an immune checkpoint inhibitor (i.e., a checkpoint inhibitor) decreases the activity of a checkpoint protein that negatively regulates immune cell function, e.g., in order to enhance T cell activation and/or an anti-cancer immune response. In other embodiments, a checkpoint inhibitor increases the activity of a checkpoint protein that positively regulates immune cell function, e.g., in order to enhance T cell activation and/or an anti-cancer immune response. In some embodiments, the checkpoint inhibitor is an antibody. Examples of checkpoint inhibitors include, without limitation, a PD-L1 axis binding antagonist (e.g., an anti-PD-L1 antibody, e.g., atezolizumab (MPDL3280A)), an antagonist directed against a co-inhibitory molecule (e.g., a CTLA4 antagonist (e.g., an anti-CTLA4 antibody), a TIM-3 antagonist (e.g., an anti-TIM-3 antibody), or a LAG-3 antagonist (e.g., an anti-LAG-3 antibody)), or any combination thereof. In some embodiments, the immune checkpoint inhibitors comprise drugs such as small molecules, recombinant forms of ligand or receptors, or antibodies, such as human antibodies (e.g., International Patent Publication WO2015016718; Pardoll, Nat Rev Cancer, 12(4): 252-64, 2012; both incorporated herein by reference). In some embodiments, known inhibitors of immune checkpoint proteins or analogs thereof may be used, in particular chimerized, humanized or human forms of antibodies may be used.

In some embodiments, the checkpoint inhibitor is a PD-L1 axis binding antagonist, e.g., a PD-1 binding antagonist, a PD-L1 binding antagonist, or a PD-L2 binding antagonist. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. PD-L1 (programmed death ligand 1) is also referred to in the art as “programmed cell death 1 ligand 1,” “PDCD1 LG1,” “CD274,” “B7-H,” and “PDL1.” An exemplary human PD-L1 is shown in UniProtKB/Swiss-Prot Accession No. Q9NZQ7.1. PD-L2 (programmed death ligand 2) is also referred to in the art as “programmed cell death 1 ligand 2,” “PDCD1 LG2,” “CD273,” “B7-DC,” “Btdc,” and “PDL2.” An exemplary human PD-L2 is shown in UniProtKB/Swiss-Prot Accession No. Q9BQ51. In some instances, PD-1, PD-L1, and PD-L2 are human PD-1, PD-L1 and PD-L2.

In some instances, the PD-1 binding antagonist is a molecule that inhibits the binding of PD-1 to its ligand binding partners. In a specific embodiment, the PD-1 ligand binding partners are PD-L1 and/or PD-L2. In another instance, a PD-L1 binding antagonist is a molecule that inhibits the binding of PD-L1 to its binding ligands. In a specific embodiment, PD-L1 binding partners are PD-1 and/or B7-1. In another instance, the PD-L2 binding antagonist is a molecule that inhibits the binding of PD-L2 to its ligand binding partners. In a specific embodiment, the PD-L2 binding ligand partner is PD-1. The antagonist may be an antibody, an antigen binding fragment thereof, an immunoadhesin, a fusion protein, or an oligopeptide. In some embodiments, the PD-1 binding antagonist is a small molecule, a nucleic acid, a polypeptide (e.g., antibody), a carbohydrate, a lipid, a metal, or a toxin.

In some instances, the PD-1 binding antagonist is an anti-PD-1 antibody (e.g., a human antibody, a humanized antibody, or a chimeric antibody), for example, as described below. In some instances, the anti-PD-1 antibody is selected from the group consisting of MDX-1 106 (nivolumab), MK-3475 (pembrolizumab, Keytruda®), MEDI-0680 (AMP-514), PDR001, REGN2810, MGA-012, JNJ-63723283, BI 754091, and BGB-108. In other instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence)). In some instances, the PD-1 binding antagonist is AMP-224.

Other examples of anti-PD-1 antibodies include, but are not limited to, MEDI-0680 (AMP-514; AstraZeneca), PDR001 (CAS Registry No. 1859072-53-9; Novartis), REGN2810 (LIBTAYO® or cemiplimab-rwlc; Regeneron), BGB-108 (BeiGene), BGB-A317 (BeiGene), BI 754091, JS-001 (Shanghai Junshi), STI-A1110 (Sorrento), INCSHR-1210 (Incyte), PF-06801591 (Pfizer), TSR-042 (also known as ANB011; Tesaro/AnaptysBio), AM0001 (ARMO Biosciences), ENUM 244C8 (Enumeral Biomedical Holdings), ENUM 388D4 (Enumeral Biomedical Holdings). In some embodiments, the PD-1 axis binding antagonist comprises tislelizumab (BGB-A317), BGB-108, STI-A1110, AM0001, BI 754091, sintilimab (IBI308), cetrelimab (JNJ-63723283), toripalimab (JS-001), camrelizumab (SHR-1210, INCSHR-1210, HR-301210), MEDI-0680 (AMP-514), MGA-012 (INCMGA 0012), nivolumab (BMS-936558, MDX1106, ONO-4538), spartalizumab (PDR001), pembrolizumab (MK-3475, SCH 900475, Keytruda®), PF-06801591, cemiplimab (REGN-2810, REGEN2810), dostarlimab (TSR-042, ANB011), FITC-YT-16 (PD-1 binding peptide), APL-501 or CBT-501 or genolimzumab (GB-226), AB-122, AK105, AMG 404, BCD-100, F520, HLX10, HX008, JTX-4014, LZM009, Sym021, PSB205, AMP-224 (fusion protein targeting PD-1), CX-188 (PD-1 probody), AGEN-2034, GLS-010, budigalimab (ABBV-181), AK-103, BAT-1306, CS-1003, AM-0001, TILT-123, BH-2922, BH-2941, BH-2950, ENUM-244C8, ENUM-388D4, HAB-21, H EISCOI 11-003, IKT-202, MCLA-134, MT-17000, PEGMP-7, PRS-332, RXI-762, STI-1110, VXM-10, XmAb-23104, AK-112, HLX-20, SSI-361, AT-16201, SNA-01, AB122, PD1-PIK, PF-06936308, RG-7769, CAB PD-1 Abs, AK-123, MEDI-3387, MEDI-5771, 4H1128Z-E27, REMD-288, SG-001, BY-24.3, CB-201, IBI-319, ONCR-177, Max-1, CS-4100, JBI-426, CCC-0701, CCX-4503, or a derivative thereof.

In some embodiments, the PD-L1 binding antagonist is a small molecule that inhibits PD-1. In some embodiments, the PD-L1 binding antagonist is a small molecule that inhibits PD-L1. In some embodiments, the PD-L1 binding antagonist is a small molecule that inhibits PD-L1 and VISTA or PD-L1 and TIM3. In some embodiments, the PD-L1 binding antagonist is CA-170 (also known as AUPM-170). In any of the instances herein, the isolated anti-PD-L1 antibody can bind to a human PD-L1, for example a human PD-L1 as shown in UniProtKB/Swiss-Prot Accession No. Q9NZQ7.1, or a variant thereof. In some embodiments, the PD-L1 binding antagonist is a small molecule, a nucleic acid, a polypeptide (e.g., antibody), a carbohydrate, a lipid, a metal, or a toxin.

In some instances, the PD-L1 binding antagonist is an anti-PD-L1 antibody, for example, as described below. In some instances, the anti-PD-L1 antibody is capable of inhibiting binding between PD-L1 and PD-1 and/or between PD-L1 and B7-1. In some instances, the anti-PD-L1 antibody is a monoclonal antibody. In some instances, the anti-PD-L1 antibody is an antibody fragment selected from the group consisting of Fab, Fab′-SH, Fv, scFv, and (Fab′)2 fragments. In some instances, the anti-PD-L1 antibody is a humanized antibody. In some instances, the anti-PD-L1 antibody is a human antibody. In some instances, the anti-PD-L1 antibody is selected from the group consisting of YW243.55.570, MPDL3280A (atezolizumab), MDX-1 105, MEDI4736 (durvalumab), and MSB0010718C (avelumab). In some embodiments, the PD-L1 axis binding antagonist comprises atezolizumab, avelumab, durvalumab (imfinzi), BGB-A333, SHR-1316 (HTI-1088), CK-301, BMS-936559, envafolimab (KN035, ASC22), CS1001, MDX-1105 (BMS-936559), LY3300054, STI-A1014, FAZ053, CX-072, INCB086550, GNS-1480, CA-170, CK-301, M-7824, HTI-1088 (HTI-131, SHR-1316), MSB-2311, AK-106, AVA-004, BBI-801, CA-327, CBA-0710, CBT-502, FPT-155, IKT-201, IKT-703, 10-103, JS-003, KD-033, KY-1003, MCLA-145, MT-5050, SNA-02, BCD-135, APL-502 (CBT-402 or TQB2450), IMC-001, KD-045, INBRX-105, KN-046, IMC-2102, IMC-2101, KD-005, IMM-2502, 89Zr-CX-072, 89Zr-DFO-6E11, KY-1055, MEDI-1109, MT-5594, SL-279252, DSP-106, Gensci-047, REMD-290, N-809, PRS-344, FS-222, GEN-1046, BH-29xx, FS-118, or a derivative thereof.

In some embodiments, the checkpoint inhibitor is an antagonist of CTLA4. In some embodiments, the checkpoint inhibitor is a small molecule antagonist of CTLA4. In some embodiments, the checkpoint inhibitor is an anti-CTLA4 antibody. CTLA4 is part of the CD28-B7 immunoglobulin superfamily of immune checkpoint molecules that acts to negatively regulate T cell activation, particularly CD28-dependent T cell responses. CTLA4 competes for binding to common ligands with CD28, such as CD80 (B7-1) and CD86 (B7-2), and binds to these ligands with higher affinity than CD28. Blocking CTLA4 activity (e.g., using an anti-CTLA4 antibody) is thought to enhance CD28-mediated costimulation (leading to increased T cell activation/priming), affect T cell development, and/or deplete Tregs (such as intratumoral Tregs). In some embodiments, the CTLA4 antagonist is a small molecule, a nucleic acid, a polypeptide (e.g., antibody), a carbohydrate, a lipid, a metal, or a toxin. In some embodiments, the CTLA-4 inhibitor comprises ipilimumab (IBI310, BMS-734016, MDX010, MDX-CTLA4, MEDI4736), tremelimumab (CP-675, CP-675,206), APL-509, AGEN1884, CS1002, AGEN1181, Abatacept (Orencia, BMS-188667, RG2077), BCD-145, ONC-392, ADU-1604, REGN4659, ADG116, KN044, KN046, or a derivative thereof.

In some embodiments, the immune checkpoint inhibitor comprises a LAG-3 inhibitor (e.g., an antibody, an antibody conjugate, or an antigen-binding fragment thereof). In some embodiments, the LAG-3 inhibitor comprises a small molecule, a nucleic acid, a polypeptide (e.g., an antibody), a carbohydrate, a lipid, a metal, or a toxin. In some embodiments, the LAG-3 inhibitor comprises a small molecule. In some embodiments, the LAG-3 inhibitor comprises a LAG-3 binding agent. In some embodiments, the LAG-3 inhibitor comprises an antibody, an antibody conjugate, or an antigen-binding fragment thereof. In some embodiments, the LAG-3 inhibitor comprises eftilagimod alpha (IMP321, IMP-321, EDDP-202, EOC-202), relatlimab (BMS-986016), GSK2831781 (IMP-731), LAG525 (IMP701), TSR-033, EVIP321 (soluble LAG-3 protein), BI 754111, IMP761, REGN3767, MK-4280, MGD-013, XmAb22841, INCAGN-2385, ENUM-006, AVA-017, AM-0003, iOnctura anti-LAG-3 antibody, Arcus Biosciences LAG-3 antibody, Sym022, a derivative thereof, or an antibody that competes with any of the preceding.

In some embodiments, the anti-cancer agent comprises an immunoregulatory molecule or cytokine. An immunoregulatory profile is required to trigger an efficient immune response and balance the immunity in a subject. In some embodiments, the immunoregulatory molecule is in included with any of the treatments detailed herein. Examples of suitable immunoregulatory cytokines include, but are not limited to, interferons (e.g., IFNα, IFNβ and IFNγ), interleukins (e.g., IL-1, IL-2, IL-3, IL-4, IL-5, IL-6, IL-7, IL-8, IL-9, IL-10, IL-12 and IL-20), tumor necrosis factors (e.g., TNFα and TNFβ), erythropoietin (EPO), FLT-3 ligand, gIp10, TCA-3, MCP-1, MIF, MIP-1α, MIP-1β, Rantes, macrophage colony stimulating factor (M-CSF), granulocyte colony stimulating factor (G-CSF), and granulocyte-macrophage colony stimulating factor (GM-CSF), as well as functional fragments thereof. In some embodiments, any immunomodulatory chemokine that binds to a chemokine receptor, i.e., a CXC, CC, C, or CX3C chemokine receptor, can be used in the context of the present disclosure. Examples of chemokines include, but are not limited to, MIP-3α (Lax), MIP-313, Hcc-1, MPIF-1, MPIF-2, MCP-2, MCP-3, MCP-4, MCP-5, Eotaxin, Tarc, Elc, I309, IL-8, GCP-2 Groα, Gro-β, Nap-2, Ena-78, Ip-10, MIG, I-Tac, SDF-1, and BCA-1 (Bic), as well as functional fragments thereof.

In some embodiments, an anti-cancer agent is administered to an individual having a PD-L1 positive ILC metastasis or an ILC metastasis with a high tumor mutational burden, e.g., of at least about 10 mut/Mb. In some embodiments, the anti-cancer agent administered to an individual having a PD-L1 positive ILC metastasis or an ILC metastasis with a high tumor mutational burden, e.g., of at least about 10 mut/Mb, comprises an immune checkpoint inhibitor. In some embodiments, the immune checkpoint inhibitor is a PD-1 binding antagonist. In some embodiments, the PD-1 binding antagonist is an anti-PD-1 antibody or fragment thereof. In some embodiments, the anti-PD-1 antibody or antibody fragment is MDX-1106 (nivolumab), MK-3475 (pembrolizumab, Keytruda®), MEDI-0680 (AMP-514), PDR001, REGN2810, MGA-012, JNJ-63723283, BI 754091, BGB-108, BGB-A317, JS-001, STI-A1110, INCSHR-1210, PF-06801591, TSR-042, AM0001, ENUM 244C8, or ENUM 388D4. In some embodiments, the PD-1 binding antagonist is an anti-PD-1 immunoadhesin. In some embodiments, the anti-PD-1 immunoadhesin is AMP-224. In other embodiments, the immune checkpoint inhibitor is a PD-L1 binding antagonist. In some embodiments, the PD-L1 binding antagonist is an anti-PD-L1 antibody or fragment thereof. In some embodiments, the anti-PD-L1 antibody or antibody fragment is YW243.55.S70, MPDL3280A (atezolizumab), MDX-1105, MEDI4736 (durvalumab), MSB0010718C (avelumab), LY3300054, STI-A1014, KN035, FAZ053, or CX-072.

In some embodiments, an anti-cancer agent is administered to an individual having an ILC metastasis with an alteration in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D. In some embodiments, the alterations are deletions, insertions, single nucleotide variants, copy number variations, rearrangements or gene fusions. In some embodiments, the anti-cancer agent is a small molecule inhibitor, a chemotherapy, an antibody or antibody fragment, or a nucleic acid. In some embodiments, the anti-cancer agent is administered in combination with a second anti-cancer agent.

In some embodiments, the anti-cancer agent administered to an individual having an ILC metastasis comprising an alteration in PIK3CA comprises a kinase inhibitor. In some embodiments, the kinase inhibitor is GSK2636771, buparlisib (BKM120), AZD8186, copanlisib (BAY80-6946), LY294002, PX-866, TGX115, TGX126, BEZ235, SF1126, idelalisib, pictilisib (GDC-094), GDC0032, IP1145, INK1117 (MLN1117), SAR260301, KIN-193 (AZD6482), GS-9820, GSK2636771, GDC-0980, AMG319, pazobanib, or alpelisib (BYL719, Piqray). In some embodiments, the anti-cancer agent administered to an individual having an ILC metastasis comprising an alteration in PIK3CA comprises an AKT inhibitor. In some embodiments, the AKT inhibitor is capivasertib, ARQ751, API-59-OMe, perifosine (KRX-0401), A-443654, PX-316, ipatasertib (GDC-0068), GSK690693, or MK2206. In some embodiments, the anti-cancer agent administered to an individual having an ILC metastasis comprising an alteration in PIK3CA is an EZH2 inhibitor. In some embodiments, the EZH2 inhibitor is GSK126, ABT263, tazemetostat, or CPI1205. In some embodiments, the anti-cancer agent administered to an individual having an ILC metastasis comprising an alteration in PIK3CA is an mTOR inhibitor. In some embodiments, the mTOR inhibitor is AP-23573. In some embodiments, the anti-cancer agent administered to an individual having an ILC metastasis comprising an alteration in PIK3CA is administered in combination with a second anti-cancer agent. In some embodiments, the second anti-cancer agent is a chemotherapeutic agent, an mTOR inhibitor, or an EGFR inhibitor.

In some embodiments, the anti-cancer agent administered to an individual having an ILC metastasis comprising an alteration in BRCA1 or BRCA2 comprises a Poly (ADP) ribose polymerase (PARP) inhibitor. In some embodiments, the PARP inhibitor is tazolaparib, veliparib, niraparib, olaparib, or rucaparib (Rubraca®). In some embodiments, the anti-cancer agent administered to an individual having an ILC metastasis comprising an alteration in BRCA1 or BRCA2 comprises a Chk1/2 inhibitor. In some embodiments, the Chk1/2 inhibitor is LY2606368. In some embodiments, the anti-cancer agent administered to an individual having an ILC metastasis comprising an alteration in BRCA1 or BRCA2 comprises a Wee1 inhibitor. In some embodiments, the Wee1 inhibitor is AZD1775. In some embodiments, the anti-cancer agent administered to an individual having an ILC metastasis comprising an alteration in BRCA1 or BRCA2 is administered in combination with a second anti-cancer agent. In some embodiments, the second anti-cancer agent is chemotherapeutic agent. In some embodiments, the chemotherapeutic agent is a platinum-based chemotherapeutic agent. In some embodiments, the second anti-cancer agent is an anti-angiogenic agent.

In some embodiments, the anti-cancer agent administered to an individual having an ILC metastasis comprising an alteration in ESR1 comprises a Selective Estrogen Receptor Modulator (SERM). Exemplary SERMs include raloxifene (Evista®), EM652, GW7604, keoxifene, toremifene (Fareston®), tamoxifen (Nolvadex®), lasofoxifene, levormeloxifene, bazedoxifene, and arzoxifene. In other embodiments, the anti-cancer agent administered to an individual having an ILC metastasis comprising an alteration in ESR1 comprises an anti-estrogen agent. In some embodiments, the anti-estrogen agent is fulvestrant. In other embodiments, the anti-cancer agent administered to an individual having an ILC metastasis comprising an alteration in ESR1 comprises an aromatase inhibitor. In some embodiments, the aromatase inhibitor is aminoglutethimide, testolactone (Teslac®), anastrozole (Arimidex®), letrozole (Femara®), exemestane (Aromasin®), vorozole (Rivizor), formestane (Lentaron®), fadrozole (Afema), 4-hydroxyandrostenedione, 1,4,6-androstatrien-3, 17-dione (ATD), or 4-Androstene-3,6,17-trione (6-OXO).

In some embodiments, the anti-cancer agent administered to an individual having an ILC metastasis comprising an alteration in KRAS comprises a kinase inhibitor. In some embodiments, the kinase inhibitor is MCP1, SCH54292, tipifarnib (R115777), lonafarnib (SCH663366), or BMS-214662. In other embodiments, the anti-cancer agent administered to an individual having an ILC metastasis comprising an alteration in KRAS comprises a farnesyltransferase inhibitor, a geranylgeranyltransferase inhibitor, a palmitoylation inhibitor, an inhibitor of methylation cleavage, or an agent that inhibits the modification or post-translational processing of KRAS. In other embodiments, the anti-cancer agent administered to an individual having an ILC metastasis comprising an alteration in KRAS is a Raf inhibitor, a MEK inhibitor, or an mTOR inhibitor.

In some embodiments, the anti-cancer agent administered to an individual having an ILC metastasis comprising an alteration in ERBB2 comprises a kinase inhibitor, a small molecule, an antibody or antibody fragment, or a cellular immunotherapy. In some embodiments, the kinase inhibitor is a multi-specific kinase inhibitor, a reversible HER2 inhibitor, an irreversible HER2 inhibitor, a pan-ERBB inhibitor, a dual HER2 inhibitor, a HER2-specific inhibitor, an EGFR inhibitor, or a dual EGFR/ERBB inhibitor. In some embodiments, the kinase inhibitor is afatinib, TAK-285, neratinib, dacomitinib, BMS-690514, BMS-599626, pelitinib, CP-724714, lapatinib, TAK-165, ARRY-380, or AZD8931. In other embodiments, the anti-cancer agent comprises an antibody or antibody fragment. In some embodiments, the antibody or antibody fragment is AV-203, AMG-888, MM-111, MM-121, MM-141, LJM716, REGN1400, MEHD7945A, RG7116, trastuzumab, trastuzumab emtansine (T-DM1), or pertuzumab. In other embodiments, the anti-cancer agent comprises a cellular immunotherapy. In some embodiments, the cellular immunotherapy is APC 8024.

In some embodiments, the anti-cancer agent administered to an individual having an ILC metastasis comprising an alteration in BRAF comprises a kinase inhibitor. In some embodiments, the kinase inhibitor is sorafenib, PLX4720, PLX-3603, dabrafenib (GSK2118436), encorafenib (LGX818), GDC-0879, RAF265, XL281, ARQ736, BAY73-4506, vemurafenib (Zelboraf®), erlotinib (Tarceva®), or BAY 43-9006.

In some embodiments, the anti-cancer agent administered to an individual having an ILC metastasis comprising an alteration in ARID1A comprises a PARP inhibitor. In some embodiments, the PARP inhibitor is olaparib (AZD2281, KU-0059436, Lynparza®), niraparib, BGB-A317, or BMN673. In some embodiments, the anti-cancer agent administered to an individual having an ILC metastasis comprising an alteration in ARID1A comprises a Bromodomain-containing protein 4 (BRD4) inhibitor. In some embodiments, the BRD4 inhibitor is PLX2853. In some embodiments, the anti-cancer agent administered to an individual having an ILC metastasis comprising an alteration in ARID1A comprises an HDAC inhibitor. In some embodiments, the HDAC inhibitor is ACY1215 (rocilinostat), CAY10603, belinostat, or SAHA (vorinostat, suberoylanilide hydroxamine). In some embodiments, the anti-cancer agent administered to an individual having an ILC metastasis comprising an alteration in ARID1A comprises a PI3K inhibitor. In some embodiments, the PI3K inhibitor is buparsilib (BKM120), idelalisib (GS-1101, CAL-101), copanlisib, or duvelisib. In some embodiments, the anti-cancer agent administered to an individual having an ILC metastasis comprising an alteration in ARID1A comprises an ATR inhibitor. In some embodiments, the ATR inhibitor is M6620, or AZD6738. In some embodiments, the anti-cancer agent administered to an individual having an ILC metastasis comprising an alteration in ARID1A comprises an EZH2 inhibitor. In some embodiments, the EZH2 inhibitor is GSK126, ABT263, tazemetostat, or CPI1205. In some embodiments, the anti-cancer agent administered to an individual having an ILC metastasis comprising an alteration in ARID1A comprises an AKT inhibitor. In some embodiments, the AKT inhibitor is AZD5363, perifosine (KRX-0401), MK2206, or GSK690693. In some embodiments, the anti-cancer agent administered to an individual having an ILC metastasis comprising an alteration in ARID1A comprises a TrxR inhibitor or a GSH inhibitor. In some embodiments, the anti-cancer agent administered to an individual having an ILC metastasis comprising an alteration in ARID1A comprises an immune checkpoint inhibitor. In some embodiments, the immune checkpoint inhibitor is an inhibitor of PD-1 or PD-L1. In other embodiments, the anti-cancer agent administered to an individual having an ILC metastasis comprising an alteration in ARID1A is administered in combination with a second anti-cancer agent. In some embodiments, the second anti-cancer agent is an immune checkpoint inhibitor, an ATR inhibitor, a PARP inhibitor, or a PI3K inhibitor.

In some embodiments, the anti-cancer agent administered to an individual having an ILC metastasis comprising an alteration in PTEN comprises a PI3K inhibitor. In some embodiments, the PI3K inhibitor is GSK2636771, buparlisib (BKM120), AZD8186, copanlisib (BAY80-6946), LY294002, PX-866, TGX115, TGX126, BEZ235, SF1126, idelalisib (GS-1101, CAL-101), pictilisib (GDC-094), GDC0032, IPI145, INK1117 (MLN1117), SAR260301, KIN-193 (AZD6482), GS-9820, GSK2636771, GDC-0980, AMG319, pazobanib, or alpelisib (BYL719, Piqray). In some embodiments, the anti-cancer agent administered to an individual having an ILC metastasis comprising an alteration in PTEN comprises an AKT inhibitor. In some embodiments, the AKT inhibitor is capivasertib, ARQ751, API-59-OMe, perifosine (KRX-0401), A-443654, PX-316, ipatasertib (GDC-0068), or MK2206. In some embodiments, the anti-cancer agent administered to an individual having an ILC metastasis comprising an alteration in PTEN comprises an mTOR inhibitor. In some embodiments, the mTOR inhibitor is AP-23573, everolimus (RAD001), or CCI-779. In some embodiments, the anti-cancer agent administered to an individual having an ILC metastasis comprising an alteration in PTEN comprises a MET inhibitor. In some embodiments, the MET inhibitor is capmatinib (INC280). In some embodiments, the anti-cancer agent administered to an individual having an ILC metastasis comprising an alteration in PTEN is administered in combination with a second anti-cancer agent. In some embodiments, the second anti-cancer agent is a hormone therapy agent, a chemotherapeutic agent, an mTOR inhibitor, or an EGFR inhibitor.

In some embodiments, the anti-cancer agent administered to an individual having an ILC metastasis comprising an alteration in FGFR2 comprises a kinase inhibitor. In some embodiments, the kinase inhibitor is formononetin, AZ12908010, R04383596, Ki23057, SU5402, E3810, PRN1371, RLY-4008, and XL-228. In some embodiments, the anti-cancer agent administered to an individual having an ILC metastasis comprising an alteration in FGFR2 is administered in combination with a second anti-cancer agent. In some embodiments, the second anti-cancer agent is a Bcl-2 inhibitor, an AKT inhibitor, an mTOR inhibitor, an HSP90 inhibitor, or a FUS inhibitor.

In some embodiments, the anti-cancer agent administered to an individual having an ILC metastasis comprising an alteration in NF1 comprises a kinase inhibitor. In some embodiments, the kinase inhibitor is trametinib, TAS0612, cabozantinib, or LY3214996. In other embodiments, the anti-cancer agent administered to an individual having an ILC metastasis comprising an alteration in NF1 comprises an mTOR inhibitor, an EGFR inhibitor, a glutaminase inhibitor, or a MEK inhibitor. In some embodiments, the anti-cancer agent is an mTOR inhibitor. In some embodiments, the mTOR inhibitor is everolimus or sirulimus. In some embodiments, the EGFR inhibitor is an antibody that binds EGFR. In some embodiments, the antibody that binds EGFR is cetuximab. In some embodiments, the anti-cancer agent is a glutaminase inhibitor. In some embodiments, the glutaminase inhibitor is BPTES or CB-839. In some embodiments, the anti-cancer agent is a MEK inhibitor. In some embodiments, the MEK inhibitor is selumetinib (AZD6244, ARRY-142886) or PD0325901.

In some embodiments, the anti-cancer agent administered to an individual having an ILC metastasis comprising an alteration in APC comprises a beta-catenin inhibitor. In some embodiments, the beta-catenin inhibitor is CGP049090, PRI-724 (ICG-001), or PKF115-584. In other embodiments, the anti-cancer agent administered to an individual having an ILC metastasis comprising an alteration in APC comprises an APC inhibitor. In some embodiments, the APC inhibitor is TASIN-1.

In some embodiments, the anti-cancer agent administered to an individual having an ILC metastasis comprising an alteration in PTPN11 comprises a small molecule inhibitor. In some embodiments, the small molecule inhibitor is SPI-112, NSC-117199, NSC-87877, TNO155, RMC-4550, SHP099, chebulinic acid, or cryptotanshinone. In other embodiments, the anti-cancer agent administered to an individual having an ILC metastasis comprising an alteration in PTPN11 comprises a kinase inhibitor.

In some embodiments, the anti-cancer agent administered to an individual having an ILC metastasis comprising an alteration in TERT comprises a small molecule inhibitor, a modified nucleotide or nucleoside analog, or a G-quadruplex stabilizer. In some embodiments, the anti-cancer agent is a small molecule inhibitor. In some embodiments, the small molecule inhibitor is MST-312, BIBR1532, or eribulin (eribulin mesylate). In some embodiments, the anti-cancer agent is a modified nucleotide or nucleoside analog. In some embodiments, the anti-cancer agent is imetelstat (GRN163L), 6-thio-2′-deoxyguanosine (6-thio-dG), or 5-fluoro-2′-deoxyuridine (5-FdU) triphosphate. In other embodiments, the anti-cancer agent is a G-quadruplex stabilizer. In some embodiments, the G-quadruplex stabilizer is BRACO-19 or TMPyP4.

In some embodiments, the anti-cancer agent administered to an individual having an ILC metastasis comprising an alteration in ALK comprises a kinase inhibitor. In some embodiments, the kinase inhibitor is crizotinib, alectinib (AF802, CH5424802), ceritinib, lorlatinib, brigatinib, ensartinib (X-396), repotrectinib (TPX-005), entrectinib (RXDX-101), AZD3463, CEP-37440, belizatinib (TSR-011), ASP3026, KRCA-0008, TQ-B3139, TPX-0131, or TAE684 (NVP-TAE684). In other embodiments, the anti-cancer agent administered to an individual having an ILC metastasis comprising an alteration in ALK comprises a heat shock protein (HSP) inhibitor or a MYC inhibitor. In some embodiments, the HSP inhibitor is a HSP90 inhibitor. In some embodiments, the HSP90 inhibitor is ganetespib. In other embodiments, the anti-cancer agent administered to an individual having an ILC metastasis comprising an alteration in ALK is administered in combination with a second anti-cancer agent. In some embodiments, the second anti-cancer agent is an immune checkpoint inhibitor, a VEGF inhibitor, an Integrin β3 inhibitor, a statin, an EGFR inhibitor, an mTOR inhibitor, a PI3K inhibitor, a MAPK inhibitor, or a CDK4/6 inhibitor.

In some embodiments, the anti-cancer agent administered to an individual having an ILC metastasis comprising an alteration in RB1 comprises a SOX2 inhibitor, an EZH2 inhibitor, a chemotherapy (e.g., a taxane), a checkpoint kinase (CHK) inhibitor, a CDC25 phosphatase inhibitor, a polo-like kinase (PLK) inhibitor, or an aurora kinase (AURK) inhibitor (e.g., LY3295668).

In some embodiments, the anti-cancer agent administered to an individual having an ILC metastasis comprising an alteration in SMAD4 comprises a PARP inhibitor. In some embodiments, the PARP inhibitor is olaparib (AZD2281, KU-0059436, Lynparza®).

In some embodiments, a kinase inhibitor for use according to any of the methods provided herein may be any suitable kinase inhibitor known in the art, such as PHA-739358, nilotinib, dasatinib, PD166326, NSC 743411, lapatinib (GW-572016), canertinib (CI-1033), semaxinib (SU5416), vatalanib (PTK787/ZK222584), sutent (SU1 1248), sorafenib (BAY 43-9006), leflunomide (SU101), imatinib (Gleevec/Glivec), or gefitinib (Iressa). For a discussion of kinase inhibitors, see e.g. Zhang et al. Nature Reviews Cancer (2009) 9:28-39.

In some instances, the anti-cancer agent is administered as a monotherapy.

In some embodiments of the present disclosure, the methods further comprise administering to the individual an additional anti-cancer therapy. In some embodiments, the additional anti-cancer therapy comprises one or more of surgery, radiotherapy, chemotherapy, anti-angiogenic therapy, anti-DNA repair therapy, and anti-inflammatory therapy. In some instances, the additional anti-cancer therapy comprises an anti-neoplastic agent, a chemotherapeutic agent, a growth inhibitory agent, an anti-angiogenic agent, a radiation therapy, a cytotoxic agent, or combinations thereof. In some instances, an anti-cancer agent may be administered in conjunction with a chemotherapy or chemotherapeutic agent. In some embodiments, the chemotherapy or chemotherapeutic agent is a platinum-based agent (including without limitation cisplatin, carboplatin, oxaliplatin, and staraplatin). In some instances, an anti-cancer agent may be administered in conjunction with a radiation therapy.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammal1 and calicheamicin omegal1); dynemicin, including dynemicin A; bisphosphonates, such as clodronate; an esperamicin; as well as neocarzinostatin chromophore and related chromoprotein enediyne antiobiotic chromophores, aclacinomy sins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenishers such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylomithine (DMFO); retinoids, such as retinoic acid; capecitabine; carboplatin, procarbazine, plicomycin, gemcitabine, navelbine, famesyl-protein tansferase inhibitors, transplatinum, and pharmaceutically acceptable salts, acids, or derivatives of any of the above.

Some (non-limiting) examples of chemotherapeutic drugs which can be combined with anti-cancer agents of the present disclosure are carboplatin (Paraplatin), cisplatin (Platinol, Platinol-AQ), cyclophosphamide (Cytoxan, Neosar), docetaxel (Taxotere), doxorubicin (Adriamycin), erlotinib (Tarceva), etoposide (VePesid), fluorouracil (5-FU), gemcitabine (Gemzar), imatinib mesylate (Gleevec), irinotecan (Camptosar), methotrexate (Folex, Mexate, Amethopterin), paclitaxel (Taxol, Abraxane), sorafinib (Nexavar), sunitinib (Sutent), topotecan (Hycamtin), vincristine (Oncovin, Vincasar PFS), and vinblastine (Velban).

In some embodiments, an anti-cancer agent provided herein is administered in combination with a kinase inhibitor, such as a kinase inhibitor that targets one or more receptor tyrosine kinases, e.g., BCR-ABL, B-Raf, EGFR, HER-2/ErbB2, IGF-IR, PDGFR-α, PDGFR-β, cKit, Flt-4, Flt3, FGFR1, FGFR3, FGFR4, CSF1R, c-Met, RON, c-Ret, or ALK; one or more cytoplasmic tyrosine kinases, e.g., c-SRC, c-YES, Abl, or JAK-2; one or more serine/threonine kinases, e.g., ATM, Aurora A & B, CDKs, mTOR, PKCi, PLKs, b-Raf, S6K, or STK11/LKB1; or one or more lipid kinases, e.g., PI3K or SKI. Small molecule kinase inhibitors include PHA-739358, nilotinib, dasatinib, PD166326, NSC 743411, lapatinib (GW-572016), canertinib (CI-1033), semaxinib (SU5416), vatalanib (PTK787/ZK222584), sutent (SU1 1248), sorafenib (BAY 43-9006), or leflunomide (SU101). Additional non-limiting examples of tyrosine kinase inhibitors include imatinib (Gleevec/Glivec) and gefitinib (Iressa).

In some embodiments, the additional anti-cancer therapy comprises an anti-angiogenic agent. Angiogenesis inhibitors prevent the extensive growth of blood vessels (angiogenesis) that tumors require to survive. The angiogenesis promoted by tumor cells to meet their increasing nutrient and oxygen demands for example can be blocked by targeting different molecules. Non-limiting examples of angiogenesis-mediating molecules or angiogenesis inhibitors which may be combined with the anti-cancer agents of the present disclosure include soluble VEGF (for example: VEGF isoforms, e.g., VEGF121 and VEGF165; VEGF receptors, e.g., VEGFR1, VEGFR2; and co-receptors, e.g., Neuropilin-1 and Neuropilin-2), NRP-1, angiopoietin 2, TSP-1 and TSP-2, angiostatin and related molecules, endostatin, vasostatin, calreticulin, platelet factor-4, TIMP and CDAI, Meth-1 and Meth-2, IFNα, IFN-β and IFN-γ, CXCL10, IL-4, IL-12 and IL-18, prothrombin (kringle domain-2), antithrombin III fragment, prolactin, VEGI, SPARC, osteopontin, maspin, canstatin, proliferin-related protein, restin and drugs such as bevacizumab, itraconazole, carboxyamidotriazole, TNP-470, CM101, IFN-α platelet factor-4, suramin, SU5416, thrombospondin, VEGFR antagonists, angiostatic steroids and heparin, cartilage-derived angiogenesis inhibitory factor, matrix metalloproteinase inhibitors, 2-methoxyestradiol, tecogalan, tetrathiomolybdate, thalidomide, thrombospondin, prolactina v β3 inhibitors, linomide, and tasquinimod. In some embodiments, known therapeutic candidates that may be used in combination with the anti-cancer agents of the disclosure include naturally occurring angiogenic inhibitors, including without limitation, angiostatin, endostatin, and platelet factor-4. In another embodiment, therapeutic candidates that may be used in combination with the anti-cancer agents of the disclosure include, without limitation, specific inhibitors of endothelial cell growth, such as TNP-470, thalidomide, and interleukin-12. Still other anti-angiogenic agents that may be used in combination with the anti-cancer agents of the disclosure include those that neutralize angiogenic molecules, including without limitation, antibodies to fibroblast growth factor, antibodies to vascular endothelial growth factor, antibodies to platelet derived growth factor, or antibodies or other types of inhibitors of the receptors of EGF, VEGF or PDGF. In some embodiments, anti-angiogenic agents that may be used in combination with the anti-cancer agents of the disclosure include, without limitation, suramin and its analogs, and tecogalan. In other embodiments, anti-angiogenic agents that may be used in combination with the anti-cancer agents of the disclosure include, without limitation, agents that neutralize receptors for angiogenic factors or agents that interfere with vascular basement membrane and extracellular matrix, including, without limitation, metalloprotease inhibitors and angiostatic steroids. Another group of anti-angiogenic compounds that may be used in combination with the anti-cancer agents of the disclosure includes, without limitation, anti-adhesion molecules, such as antibodies to integrin alpha v beta 3. Still other anti-angiogenic compounds or compositions that may be used in combination with the anti-cancer agents of the disclosure include, without limitation, kinase inhibitors, thalidomide, itraconazole, carboxyamidotriazole, CM101, IFN-α, IL-12, SU5416, thrombospondin, cartilage-derived angiogenesis inhibitory factor, 2-methoxyestradiol, tetrathiomolybdate, thrombospondin, prolactin, and linomide. In one particular embodiment, the anti-angiogenic compound that may be used in combination with the anti-cancer agents of the disclosure is an antibody to VEGF, such as Avastin®/bevacizumab (Genentech).

In some embodiments, the additional anti-cancer therapy comprises an anti-DNA repair therapy, such as a PARP inhibitor, a RAD51 inhibitor, or an inhibitor of a DNA damage response kinase, e.g., CHCK1, ATM, or ATR. In some embodiments, the additional anti-cancer therapy comprises a radiosensitizer. Exemplary radiosensitizers include hypoxia radiosensitizers such as misonidazole, metronidazole, and trans-sodium crocetinate, a compound that helps to increase the diffusion of oxygen into hypoxic tumor tissue. The radiosensitizer can also be a DNA damage response inhibitor interfering with base excision repair (BER), nucleotide excision repair (NER), mismatch repair (MMR), recombinational repair comprising homologous recombination (HR) and non-homologous end joining (NHEJ), and direct repair mechanisms. Single strand break (SSB) repair mechanisms include BER, NER, or MMR pathways, while double stranded break (DSB) repair mechanisms consist of HR and NHEJ pathways. Radiation causes DNA breaks that, if not repaired, are lethal. SSBs are repaired through a combination of BER, NER and MMR mechanisms using the intact DNA strand as a template. The predominant pathway of SSB repair is BER, utilizing a family of related enzymes termed poly-(ADP-ribose) polymerases (PARP). Thus, the radiosensitizer can include DNA damage response inhibitors such as PARP inhibitors. In some embodiments, the additional anti-cancer therapy is a DNA repair and response pathway inhibitor, a PARP inhibitor (e.g., talazoparib, rucaparib, olaparib), a RAD51 inhibitor (e.g., RI-1), or an inhibitor of DNA damage response kinases such as CHCK1 (e.g., AZD7762), ATM (e.g., KU-55933, KU-60019, NU7026, or VE-821), and ATR (e.g., NU7026).

In some embodiments, the additional anti-cancer therapy comprises an anti-inflammatory agent. In some embodiments, the anti-inflammatory agent is an agent that blocks, inhibits, or reduces inflammation or signaling from an inflammatory signaling pathway. In some embodiments, the anti-inflammatory agent inhibits or reduces the activity of one or more of any of the following: IL-1, IL-2, IL-3, IL-4, IL-5, IL-6, IL-7, IL-8, IL-9, IL-10, IL-12, IL-13, IL-15, IL-18, IL-23; interferons (IFNs), e.g., IFNα, IFNβ, IFNγ, IFN-γ inducing factor (IGIF); transforming growth factor-β(TGF-β); transforming growth factor-α (TGF-α); tumor necrosis factors, e.g., TNF-α, TNF-β, TNF-RI, TNF-RII; CD23; CD30; CD40L; EGF; G-CSF; GDNF; PDGF-BB; RANTES/CCLS; IKK; NF-κB; TLR2; TLR3; TLR4; TL5; TLR6; TLR7; TLR8; TLR8; TLR9; and/or any cognate receptors thereof. In some embodiments, the anti-inflammatory agent is an IL-1 or IL-1 receptor antagonist, such as anakinra (Kineret®), rilonacept, or canakinumab. In some embodiments, the anti-inflammatory agent is an IL-6 or IL-6 receptor antagonist, e.g., an anti-IL-6 antibody or an anti-IL-6 receptor antibody, such as tocilizumab (ACTEMRA®), olokizumab, clazakizumab, sarilumab, sirukumab, siltuximab, or ALX-0061. In some embodiments, the anti-inflammatory agent is a TNF-α antagonist, e.g., an anti-TNFα antibody, such as infliximab (Remicade®), golimumab (Simponi®), adalimumab (Humira®), certolizumab pegol (Cimzia®) or etanercept.

In some embodiments, the anti-inflammatory agent is a corticosteroid. Exemplary corticosteroids include, but are not limited to, cortisone (hydrocortisone, hydrocortisone sodium phosphate, hydrocortisone sodium succinate, Ala-Cort®, Hydrocort Acetate®, hydrocortone phosphate Lanacort®, Solu-Cortef®), decadron (dexamethasone, dexamethasone acetate, dexamethasone sodium phosphate, Dexasone®, Diodex®, Hexadrol®, Maxidex®), methylprednisolone (6-methylprednisolone, methylprednisolone acetate, methylprednisolone sodium succinate, Duralone®, Medralone®, Medrol®, M-Prednisol®, Solu-Medrol®), prednisolone (Delta-Cortef®, ORAPRED®, Pediapred®, Prezone®), and prednisone (Deltasone®, Liquid Pred®, Meticorten®, Orasone®), and bisphosphonates (e.g., pamidronate (Aredia®), and zoledronic acid (Zometac®).

### Software, Systems, and Devices

In some other aspects, provided herein are non-transitory computer-readable storage media. In some embodiments, the non-transitory computer-readable storage media comprise one or more programs for execution by one or more processors of a device, the one or more programs including instructions which, when executed by the one or more processors, cause the device to perform a method according to any of the embodiments described herein.

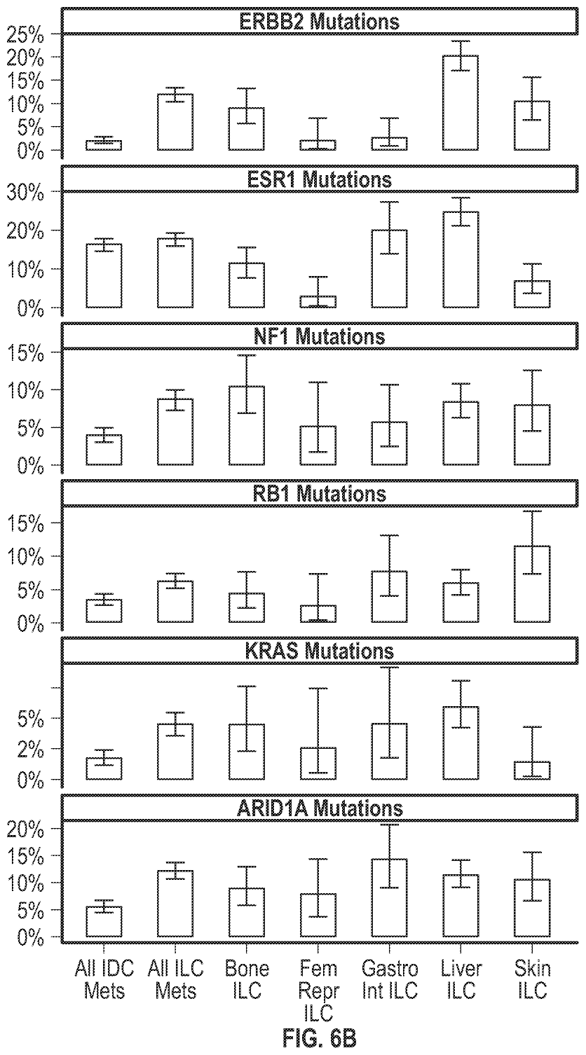

FIG. 7 illustrates an example of a computing device in accordance with one embodiment. Device 1100 can be a host computer connected to a network. Device 1100 can be a client computer or a server. As shown in FIG. 7, device 1100 can be any suitable type of microprocessor-based device, such as a personal computer, workstation, server or handheld computing device (portable electronic device) such as a phone or tablet. The device can include, for example, one or more of processor(s) 1110, input device 1120, output device 1130, storage 1140, communication device 1160, power supply 1170, operating system 1180, and system bus 1190. Input device 1120 and output device 1130 can generally correspond to those described herein, and can either be connectable or integrated with the computer.

Input device 1120 can be any suitable device that provides input, such as a touch screen, keyboard or keypad, mouse, or voice-recognition device. Output device 1130 can be any suitable device that provides output, such as a touch screen, haptics device, or speaker.

Storage 1140 can be any suitable device that provides storage (e.g., an electrical, magnetic or optical memory including a RAM (volatile and non-volatile), cache, hard drive, or removable storage disk). Communication device 1160 can include any suitable device capable of transmitting and receiving signals over a network, such as a network interface chip or device. The components of the computer can be connected in any suitable manner, such as via a wired media (e.g., a physical bus, ethernet, or any other wire transfer technology) or wirelessly (e.g., Bluetooth®, Wi-Fi®, or any other wireless technology). For example, in FIG. 7, the components are connected by System Bus 1190.

Detection module 1150, which can be stored as executable instructions in storage 1140 and executed by processor(s) 1110, can include, for example, the processes that embody the functionality of the present disclosure (e.g., as embodied in the devices as described herein).

Detection module 1150 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 1140, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Detection module 1150 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

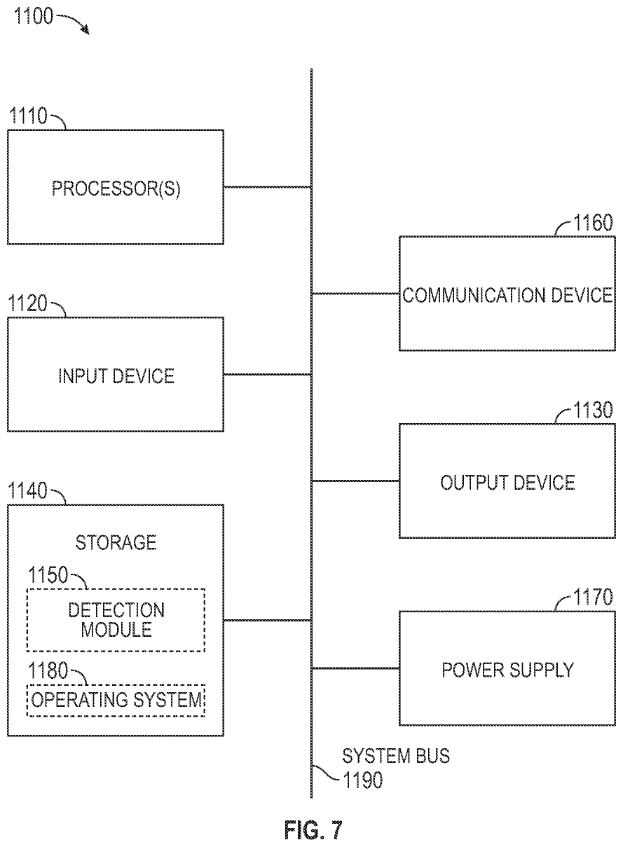

Device 1100 may be connected to a network (e.g., Network 1204, as shown in FIG. 8 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, Ti or T3 lines, cable networks, DSL, or telephone lines.

Device 1100 can implement any operating system (e.g., Operating System 1180) suitable for operating on the network. Detection module 1150 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, Operating System 1180 is executed by one or more processors, e.g., Processor(s) 1110.

Device 1100 can further include Power Supply 1170, which can be any suitable power supply.

FIG. 8 illustrates an example of a computing system in accordance with one embodiment. In System 1200, Device 1100 (e.g., as described above and illustrated in FIG. 7) is connected to Network 1204, which is also connected to Device 1206. In some embodiments, Device 1206 is a sequencer. Exemplary sequencers can include, without limitation, Roche/454's Genome Sequencer (GS) FLX System, Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio RS system. Devices 1100 and 1206 may communicate, e.g., using suitable communication interfaces via Network 1204, such as a Local Area Network (LAN), Virtual Private Network (VPN), or the Internet. In some embodiments, Network 1204 can be, for example, the Internet, an intranet, a virtual private network, a cloud network, a wired network, or a wireless network. Devices 1100 and 1206 may communicate, in part or in whole, via wireless or hardwired communications, such as Ethernet, IEEE 802.11b wireless, or the like. Additionally, Devices 1100 and 1206 may communicate, e.g., using suitable communication interfaces, via a second network, such as a mobile/cellular network. Communication between Devices 1100 and 1206 may further include or communicate with various servers such as a mail server, mobile server, media server, telephone server, and the like. In some embodiments, Devices 1100 and 1206 can communicate directly (instead of, or in addition to, communicating via Network 1204), e.g., via wireless or hardwired communications, such as Ethernet, IEEE 802.11b wireless, or the like. In some embodiments, Devices 1100 and 1206 communicate via Communications 1208, which can be a direct connection or can occur via a network (e.g., Network 1204).

One or all of Devices 1100 and 1206 generally include logic (e.g., http web server logic) or is programmed to format data, accessed from local or remote databases or other sources of data and content, for providing and/or receiving information via Network 1204 according to various examples described herein.

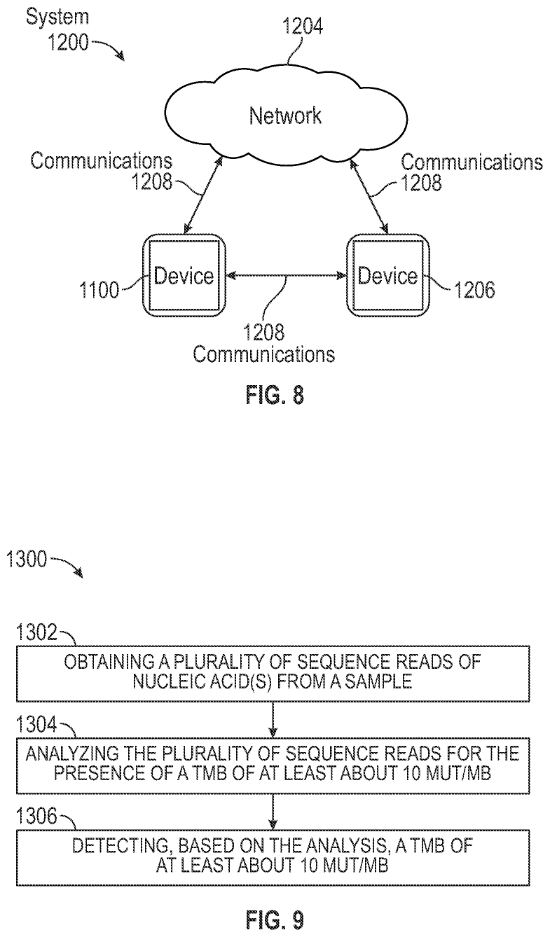

FIG. 9 illustrates an exemplary process 1300 for detecting a tumor mutational burden (TMB) of at least about 10 mutations/megabase (mut/Mb), in accordance with some embodiments. Process 1300 is performed, for example, using one or more electronic devices implementing a software program. In some examples, process 1300 is performed using a client-server system, and the blocks of process 1300 are divided up in any manner between the server and a client device. In other examples, the blocks of process 1300 are divided up between the server and multiple client devices. Thus, while portions of process 1300 are described herein as being performed by particular devices of a client-server system, it will be appreciated that process 1300 is not so limited. In some embodiments, the executed steps can be executed across many systems, e.g., in a cloud environment. In other examples, process 1300 is performed using only a client device or only multiple client devices. In process 1300, some blocks are, optionally, combined, the order of some blocks is, optionally, changed, and some blocks are, optionally, omitted. In some examples, additional steps may be performed in combination with the process 1300. Accordingly, the operations as illustrated (and described in greater detail below) are exemplary by nature and, as such, should not be viewed as limiting.

At block 1302, a plurality of sequence reads of one or more nucleic acids is obtained, wherein the one or more nucleic acids are derived from a sample obtained from an individual. In some embodiments, the sequence reads are from a sequencer, e.g., as described herein or otherwise known in the art. In some embodiments, the nucleic acid(s) comprise one or more nucleic acids from a sample obtained from an individual having an ILC metastasis, such as an ILC metastasis described herein. Optionally, prior to obtaining the sequence reads, the sample is purified, enriched, and/or subjected to PCR amplification. At block 1304, an exemplary system (e.g., one or more electronic devices) analyzes the plurality of sequence reads for the presence of a tumor mutational burden (TMB) of at least about 10 mutations/me gabase (mut/Mb). In some embodiments, the analyzing is based on between about 0.8 Mb and about 1.1 Mb of sequenced nucleic acids. At block 1306, the system detects (e.g., based on the analysis) a tumor mutational burden (TMB) of at least about 10 mutations/megabase (mut/Mb), in the sample.

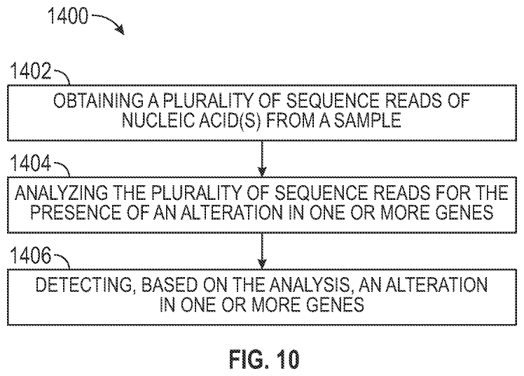

FIG. 10 illustrates an exemplary process 1400 for detecting an alteration in one or more genes, e.g., PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, CDH1 or KMT2D, in accordance with some embodiments. Process 1400 is performed, for example, using one or more electronic devices implementing a software program. In some examples, process 1400 is performed using a client-server system, and the blocks of process 1400 are divided up in any manner between the server and a client device. In other examples, the blocks of process 1400 are divided up between the server and multiple client devices. Thus, while portions of process 1400 are described herein as being performed by particular devices of a client-server system, it will be appreciated that process 1400 is not so limited. In some embodiments, the executed steps can be executed across many systems, e.g., in a cloud environment. In other examples, process 1400 is performed using only a client device or only multiple client devices. In process 1400, some blocks are, optionally, combined, the order of some blocks is, optionally, changed, and some blocks are, optionally, omitted. In some examples, additional steps may be performed in combination with the process 1400. Accordingly, the operations as illustrated (and described in greater detail below) are exemplary by nature and, as such, should not be viewed as limiting.

At block 1402, a plurality of sequence reads of one or more nucleic acids is obtained, wherein the one or more nucleic acids are derived from a sample obtained from an individual. In some embodiments, the sequence reads are from a sequencer, e.g., as described herein or otherwise known in the art. In some embodiments, the nucleic acid(s) comprise one or more nucleic acids corresponding to a PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, CDH1 or KMT2D gene of the present disclosure, or a portion thereof. In some embodiments, the nucleic acid(s) comprise one or more nucleic acids from a sample obtained from an individual having an ILC metastasis, such as an ILC metastasis described herein. Optionally, prior to obtaining the sequence reads, the sample is purified, enriched (e.g., for nucleic acid(s) corresponding to a PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, CDH1 or KMT2D gene of the present disclosure, or a portion thereof), and/or subjected to PCR amplification. At block 1404, an exemplary system (e.g., one or more electronic devices) analyzes the plurality of sequence reads for the presence of an alteration in one or more genes, e.g., PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, CDH1 or KMT2D. At block 1406, the system detects (e.g., based on the analysis) an alteration in one or more genes, e.g., PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, CDH1 or KMT2D, in the sample.

### Kits

Also provided herein are kits for detecting one or more biomarkers described herein, e.g., tumor mutational burden, PD-L1-positive status, and/or an alteration in one or more genes, in a sample from an individual having an ILC metastasis. In some embodiments, the one or more genes are selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D.

In some embodiments, a kit provided herein comprises one or more reagents (e.g., one or more oligonucleotides, primers, probes or baits of the present disclosure) for detecting a biomarker described herein, e.g., tumor mutational burden, PD-L1-positive status, and/or an alteration in one or more genes.

In some embodiments, the kit comprises one or more reagents (e.g., one or more oligonucleotides, primers, probes or baits of the present disclosure) for detecting an alteration in one or more genes, e.g., in one or more of PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D, in a sample, e.g., in a sample from an individual having an ILC metastasis described herein. Alternatively or additionally, the kit may comprise a reagent (e.g., one or more oligonucleotides, primers, probes or baits of the present disclosure) for detecting a wild-type counterpart of the one or more genes in a sample, e.g., in a sample from an individual having an ILC metastasis described herein. In some embodiments, the reagent comprises one or more oligonucleotides, primers, probes or baits of the present disclosure capable of hybridizing to a nucleic acid sequence comprising an alteration described herein, e.g., an alteration in one or more genes selected from PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D. Alternatively or additionally, the kit may comprise one or more oligonucleotides, primers, probes or baits of the present disclosure capable of hybridizing to a counterpart wild-type nucleic acid sequence of the one or more genes. In some embodiments, the reagent comprises one or more oligonucleotides, primers, probes or baits of the present disclosure capable of distinguishing a nucleic acid molecule comprising an alteration in one or more genes described herein (e.g., in one or more of PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D) from a counterpart wild-type nucleic acid molecule of the one or more genes. In some embodiments, the kit is for use according to any sequencing or nucleotide detecting assay known in the art or described herein, such as a nucleic acid hybridization assay, an amplification-based assay, a polymerase chain reaction-restriction fragment length polymorphism (PCR-RFLP) assay, real-time PCR, sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), and mass-spectrometric genotyping.

In some embodiments, the kit comprises one or more reagents (e.g., one or more antibodies) for detecting a polypeptide or a protein encoded by a gene, e.g., PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D, comprising an alteration described herein in a sample, e.g., in a sample from an individual having an ILC metastasis described herein. Alternatively or additionally, the kit may comprise a reagent (e.g., one or more antibodies) for detecting a wild-type counterpart of the polypeptide or protein in a sample, e.g., in a sample from an individual having an ILC metastasis described herein. In some embodiments, the kit comprises one or more reagents (e.g., one or more antibodies) capable of distinguishing a polypeptide or a protein encoded by a gene, e.g., PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, or KMT2D, comprising an alteration described herein from a wild-type counterpart of the polypeptide or protein in a sample, e.g., in a sample from an individual having an ILC metastasis described herein. In some embodiments, the kit is for use according to any method for detecting proteins or polypeptides known in the art or provided herein, e.g., antibody-based detection methods, mass spectrometry (e.g., tandem mass spectrometry), a reporter assay (e.g., a fluorescence-based assay), immunoblots such as a Western blot, immunoassays such as enzyme-linked immunosorbent assays (ELISA), immunohistochemistry, other immunological assays (e.g., fluid or gel precipitin reactions, immunodiffusion, immunoelectrophoresis, radioimmunoassay (RIA), immunofluorescent assays), and analytic biochemical methods (e.g., electrophoresis, capillary electrophoresis, high performance liquid chromatography (HPLC), thin layer chromatography (TLC), hyperdiffusion chromatography).

In some embodiments, the kit comprises one or more reagents (e.g., one or more oligonucleotides, primers, probes or baits of the present disclosure) for detecting or measuring the level of tumor mutational burden in a sample, e.g., in a sample from an individual having an ILC metastasis described herein. In some embodiments, the kit is for use according to any method for detecting or measuring tumor mutational burden known in the art or provided herein, e.g., whole-exome sequencing (WES), next-generation sequencing, whole genome sequencing, gene-targeted sequencing, or sequencing of a panel of genes, e.g., panels including cancer-related genes.

In some embodiments, a kit provided herein comprises one or more reagents (e.g., one or more oligonucleotides, primers, antibodies, probes or baits of the present disclosure) for detecting a PD-L1-positive status in a sample, e.g., in a sample from an individual having an ILC metastasis described herein. In some embodiments, the kit is for use according to any method for detecting a PD-L1-positive status known in the art or provided herein, e.g. using immunohistochemistry (IHC), Western blot analysis, immunoprecipitation, molecular binding assays, enzyme-linked immunosorbent assay (ELISA), enzyme-linked immunofiltration assay (ELIFA), fluorescence activated cell sorting (FACS), MassARRAY, proteomics (e.g., mass spectrometry), quantitative blood based assays (as for example serum ELISA), biochemical enzymatic activity assays, in situ hybridization, Northern analysis, polymerase chain reaction (“PCR”) including quantitative real time PCR (qRT-PCR) and other amplification-based methods, RNA-sequencing (RNA-seq), FISH, microarray analysis, gene expression profiling, and/or serial analysis of gene expression (“SAGE”). Multiplexed immunoassays such as those available from Rules Based Medicine or Meso Scale Discovery (“MSD”) may also be used. In some embodiments, the kit is suitable for use in detecting a PD-L1-positive status in sample using a VENTANA PD-L1 assay (SP142). In some embodiments, the kit comprises an anti-PD-L1 antibody suitable for use in detecting a PD-L1-positive status in sample using a VENTANA PD-L1 assay (SP142). In some embodiments, the kit is suitable for use in detecting a PD-L1-positive status in sample using a DAKO 22C3 assay. In some embodiments, the kit comprises an anti-PD-L1 antibody suitable for use in detecting a PD-L1-positive status in sample using a DAKO 22C3 assay.

In some embodiments, a kit provided herein further comprises instructions for detecting the one or more biomarkers in a sample, e.g., in a sample from an individual having an ILC metastasis described herein.

### Exemplary Embodiments

The following exemplary embodiments are representative of some aspects of the invention:

Exemplary Embodiment 1: A method of treating or delaying progression of invasive lobular carcinoma (ILC) metastasis, comprising: (a) acquiring knowledge of a tumor mutational burden (TMB) of at least about 10 mutations/megabase (mut/Mb) in a sample from an individual having an ILC metastasis; and(b) responsive to said knowledge, administering to the individual an effective amount of an immune checkpoint inhibitor.

Exemplary Embodiment 2: The method of embodiment 1, wherein the acquiring knowledge comprises measuring the level of TMB in a sample obtained from the individual.

Exemplary Embodiment 3: The method of embodiment 2, wherein TMB is measured on between about 0.8 Mb and about 1.1 Mb.

Exemplary Embodiment 4: The method of any one of embodiments 1-3, wherein the individual is a human.

Exemplary Embodiment 5: The method of any one of embodiments 1-4, wherein the ILC metastasis is selected from the group consisting of a bone ILC metastasis, a female reproductive system ILC metastasis, a gastrointestinal ILC metastasis, a liver ILC metastasis, and a skin ILC

Exemplary Embodiment 6: The method of any one of embodiments 1-5, wherein the ILC metastasis is a bone ILC metastasis.

Exemplary Embodiment 7: The method of any one of embodiments 1-5, wherein the ILC metastasis is a female reproductive system ILC metastasis.

Exemplary Embodiment 8: The method of any one of embodiments 1-5, wherein the ILC metastasis is a gastrointestinal ILC metastasis.

Exemplary Embodiment 9: The method of any one of embodiments 1-5, wherein the ILC metastasis is a liver ILC metastasis.

Exemplary Embodiment 10: The method of any one of embodiments 1-5, wherein the ILC metastasis is a skin ILC metastasis.

Exemplary Embodiment 11: The method of any one of embodiments 1-10, wherein the sample from the individual comprises fluid, cells, or tissue.

Exemplary Embodiment 12: The method of embodiment 11, wherein the sample from the individual comprises a tumor biopsy or a circulating tumor cell.

Exemplary Embodiment 13: The method of embodiment 11 or embodiment 12, wherein the sample from the individual comprises nucleic acids.

Exemplary Embodiment 14: The method of embodiment 13, wherein the sample from the individual comprises mRNA, genomic DNA, circulating tumor DNA, cell-free DNA, or cell-free RNA.

Exemplary Embodiment 15: The method of any one of embodiments 2-14, wherein TMB is measured in the sample by whole exome sequencing, whole genome sequencing, or gene-targeted sequencing.

Exemplary Embodiment 16: A method of treating or delaying progression of invasive lobular carcinoma (ILC) metastasis, comprising: (a) acquiring knowledge of a PD-L1-positive ILC metastasis in a sample from an individual having an ILC metastasis; and (b) responsive to said knowledge, administering to the individual an effective amount of an immune checkpoint inhibitor.

Exemplary Embodiment 17: The method of embodiment 16, wherein the acquiring knowledge of a PD-L1-positive ILC metastasis comprises measuring the level of PD-L1 expression in a sample obtained from the individual.

Exemplary Embodiment 18: The method of embodiment 17, wherein the level of PD-L1 expression is measured using an immunohistochemistry assay.

Exemplary Embodiment 19: The method of embodiment 17 or embodiment 18, wherein the level of PD-L1 expression is determined based on PD-L1 expression in tumor infiltrating immune cells (ICs) and/or tumor cells (TCs).

Exemplary Embodiment 20: The method of any one of embodiments 16-19, wherein the acquiring knowledge of a PD-L1-positive ILC metastasis comprises acquiring knowledge that at least about 1% of ICs in the sample are PD-L1-positive.

Exemplary Embodiment 21: The method of any one of embodiments 16-20, wherein the individual is a human.

Exemplary Embodiment 22: The method of any one of embodiments 16-21, wherein the ILC metastasis is selected from the group consisting of a bone ILC metastasis, a female reproductive system ILC metastasis, a gastrointestinal ILC metastasis, a liver ILC metastasis, and a skin ILC metastasis.

Exemplary Embodiment 23: The method of any one of embodiments 16-22, wherein the ILC metastasis is a bone ILC metastasis.

Exemplary Embodiment 24: The method of any one of embodiments 16-22, wherein the ILC metastasis is a female reproductive system ILC metastasis.

Exemplary Embodiment 25: The method of any one of embodiments 16-22, wherein the ILC metastasis is a gastrointestinal ILC metastasis.

Exemplary Embodiment 26: The method of any one of embodiments 16-22, wherein the ILC metastasis is a liver ILC metastasis.

Exemplary Embodiment 27: The method of any one of embodiments 16-22, wherein the ILC metastasis is a skin ILC metastasis.

Exemplary Embodiment 28: The method of any one of embodiments 16-27, wherein the sample from the individual comprises fluid, cells, or tissue.

Exemplary Embodiment 29: The method of embodiment 28, wherein the sample from the individual comprises a tumor biopsy or a circulating tumor cell.

Exemplary Embodiment 30: The method of any one of embodiments 1-29, wherein the immune checkpoint inhibitor is a small molecule inhibitor, an antibody or antibody fragment, a peptide, a fusion protein, or a nucleic acid.

Exemplary Embodiment 31: The method of any one of embodiments 1-30, wherein the immune checkpoint inhibitor is a PD-1 binding antagonist or a PD-L1 binding antagonist.

Exemplary Embodiment 32: The method of embodiment 31, wherein the immune checkpoint inhibitor is an anti-PD-1 antibody or antibody fragment.

Exemplary Embodiment 33: The method of embodiment 32, wherein the anti-PD-1 antibody or antibody fragment is selected from the group consisting of MDX-1106 (nivolumab), MK-3475 (pembrolizumab), MEDI-0680 (AMP-514), PDR001, REGN2810, MGA-012, JNJ-63723283, BI 754091, BGB-108, BGB-A317, JS-001, STI-A1110, INCSHR-1210, PF-06801591, TSR-042, AM0001, ENUM 244C8, and ENUM 388D4.

Exemplary Embodiment 34: The method of embodiment 32 or embodiment 33, wherein the anti-PD-1 antibody is pembrolizumab.

Exemplary Embodiment 35: The method of embodiment 31, wherein the immune checkpoint inhibitor is an anti-PD-L1 antibody or antibody fragment.

Exemplary Embodiment 36: The method of embodiment 35, wherein the anti-PD-L1 antibody or antibody fragment is selected from the group consisting of YW243.55.S70, MPDL3280A (atezolizumab), MDX-1105, MEDI4736 (durvalumab), MSB0010718C (avelumab), LY3300054, STI-A1014, KN035, FAZ053, and CX-072.

Exemplary Embodiment 37: A method of treating or delaying progression of invasive lobular carcinoma (ILC) metastasis, comprising: (a) acquiring knowledge of an alteration in one or more genes in a sample from an individual having an ILC metastasis, wherein the one or more genes are selected from the group consisting of: PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, and KMT2D; and (b) responsive to said knowledge, administering to the individual an effective amount of an anti-cancer agent.

Exemplary Embodiment 38: The method of embodiment 37, wherein the acquiring knowledge comprises detecting the alteration in the one or more genes in a sample obtained from the individual.

Exemplary Embodiment 39: The method of embodiment 37 or embodiment 38, wherein the individual is a human.

Exemplary Embodiment 40: The method of any one of embodiments 37-39, wherein the ILC metastasis is selected from the group consisting of a bone ILC metastasis, a female reproductive system ILC metastasis, a gastrointestinal ILC metastasis, a liver ILC metastasis, and a skin ILC metastasis.

Exemplary Embodiment 41: The method of any one of embodiments 37-40, wherein the ILC metastasis is a bone ILC metastasis.

Exemplary Embodiment 42: The method of any one of embodiments 37-40, wherein the ILC metastasis is a female reproductive system ILC metastasis.

Exemplary Embodiment 43: The method of any one of embodiments 37-40, wherein the ILC metastasis is a gastrointestinal ILC metastasis.

Exemplary Embodiment 44: The method of any one of embodiments 37-40, wherein the ILC metastasis is a liver ILC metastasis.

Exemplary Embodiment 45: The method of any one of embodiments 37-40, wherein the ILC metastasis is a skin ILC metastasis.

Exemplary Embodiment 46: The method of any one of embodiments 37-45, wherein the anti-cancer agent is a small molecule, a chemotherapy, an antibody or antibody fragment, a cellular immunotherapy, an immune checkpoint inhibitor, or a nucleic acid.

Exemplary Embodiment 47: The method of any one of embodiments 37-46, wherein the alteration is an alteration in PIK3CA.

Exemplary Embodiment 48: The method of embodiment 47, wherein the alteration comprises one or more of a substitution of one or more nucleotides, an insertion of one or more nucleotides, or a deletion of one or more nucleotides.

Exemplary Embodiment 49: The method of embodiment 47 or embodiment 48, wherein the alteration results in one or more amino acid substitutions in a polypeptide encoded by the PIK3CA gene at amino acid position E81, R108, K111, G118, N345, D350, E365, E418, C420, E453, P539, E542, E545, Q546, E726, E970, M1004, M1043, N1044, H1047, G1049, or any combination thereof, wherein the numbering of the residues is according to the amino acid sequence of SEQ ID NO: 26.

Exemplary Embodiment 50: The method of embodiment 49, wherein the one or more amino acid substitutions comprise a E81K, R108H, K111N, G118D, N345K, D350N, E365K, E418K, C420R, E453K, E453Q, P539R, E542K, E545K, E545A, E545Q, Q546R, Q546K, E726K, E970K, M1004I, M1043I, N1044K, H1047R, H1047L, or G1049R amino acid substitution, or any combination thereof, wherein the numbering of the residues is according to the amino acid sequence of SEQ ID NO: 26.

Exemplary Embodiment 51: The method of embodiment 47 or embodiment 48, wherein the alteration results in a deletion of amino acid residue E110 (E110del) in a polypeptide encoded by the PIK3CA gene, wherein the numbering of the residues is according to the amino acid sequence of SEQ ID NO: 26.

Exemplary Embodiment 52: The method of any one of embodiments 47-51, wherein the anti-cancer agent is a kinase inhibitor, an AKT inhibitor, an EZH2 inhibitor, or an mTOR inhibitor.

Exemplary Embodiment 53: The method of any one of embodiments 47-52, wherein the ILC metastasis is a skin ILC metastasis.

Exemplary Embodiment 54: The method of any one of embodiments 47-52, wherein the ILC metastasis is a gastrointestinal ILC metastasis.

Exemplary Embodiment 55: The method of any one of embodiments 47-52, wherein the ILC metastasis is a liver ILC metastasis.

Exemplary Embodiment 56: The method of any one of embodiments 47-52, wherein the ILC metastasis is a female reproductive system ILC metastasis.

Exemplary Embodiment 57: The method of any one of embodiments 47-52, wherein the ILC metastasis is a bone ILC metastasis.

Exemplary Embodiment 58: The method of any one of embodiments 37-46, wherein the alteration is an alteration in BRCA1 or BRCA2.

Exemplary Embodiment 59: The method of embodiment 58, wherein the anti-cancer agent is a PARP inhibitor, a Chk1/2 inhibitor, or a Wee1 inhibitor.

Exemplary Embodiment 60: The method of embodiment 58 or embodiment 59, wherein the ILC metastasis is a female reproductive system ILC metastasis.

Exemplary Embodiment 61: The method of embodiment 58 or embodiment 59, wherein the ILC metastasis is a bone ILC metastasis.

Exemplary Embodiment 62: The method of embodiment 58 or embodiment 59, wherein the ILC metastasis is a skin ILC metastasis.

Exemplary Embodiment 63: The method of embodiment 58 or embodiment 59, wherein the ILC metastasis is a liver ILC metastasis.

Exemplary Embodiment 64: The method of any one of embodiments 37-46, wherein the alteration is an alteration in ESR1.

Exemplary Embodiment 65: The method of embodiment 64, wherein the alteration comprises one or more of a substitution of one or more nucleotides, an insertion of one or more nucleotides, or a deletion of one or more nucleotides.

Exemplary Embodiment 66: The method of embodiment 65, wherein the alteration results in one or more amino acid substitutions in a polypeptide encoded by the ESR1 gene at amino acid position E380, V418, S463, V533, L536, Y537, D538, or any combination thereof, wherein the numbering of the residues is according to the amino acid sequence of SEQ ID NO: 29.

Exemplary Embodiment 67: The method of embodiment 66, wherein the one or more amino acid substitutions comprise a E380Q, V418E, S463P, V533M, L536Q, L536H, L536P, L536R, Y537S, Y537N, Y537C, Y537D, or D538G amino acid substitution, or any combination thereof, wherein the numbering of the residues is according to the amino acid sequence of SEQ ID NO: 29.

Exemplary Embodiment 68: The method of embodiment 65, wherein the alteration results in a deletion of amino acid V422 (V422del) and/or a deletion of amino acids V533-L536 (V533_L536del) in a polypeptide encoded by the ESR1 gene, wherein the numbering of the residues is according to the amino acid sequence of SEQ ID NO: 29.

Exemplary Embodiment 69: The method of any one of embodiments 64-68, wherein the anti-cancer agent is a Selective Estrogen Receptor Modulator (SERM), an anti-estrogen, or an aromatase inhibitor.

Exemplary Embodiment 70: The method of any one of embodiments 64-69, wherein the ILC metastasis is a gastrointestinal ILC metastasis.

Exemplary Embodiment 71: The method of any one of embodiments 64-69, wherein the ILC metastasis is a liver ILC metastasis.

Exemplary Embodiment 72: The method of any one of embodiments 64-69, wherein the ILC metastasis is a bone ILC metastasis.

Exemplary Embodiment 73: The method of any one of embodiments 64-69, wherein the ILC metastasis is a skin ILC metastasis.

Exemplary Embodiment 74: The method of any one of embodiments 37-46, wherein the alteration is an alteration in NF1.

Exemplary Embodiment 75: The method of embodiment 74, wherein the alteration comprises one or more of a substitution of one or more nucleotides, an insertion of one or more nucleotides, or a deletion of one or more nucleotides.

Exemplary Embodiment 76: The method of embodiment 74 or embodiment 75, wherein the anti-cancer agent is a kinase inhibitor, an mTOR inhibitor, an EGFR inhibitor, a glutaminase inhibitor, or a MEK inhibitor.

Exemplary Embodiment 77: The method of any one of embodiments 74-76, wherein the ILC metastasis is a gastrointestinal ILC metastasis.

Exemplary Embodiment 78: The method of any one of embodiments 74-76, wherein the ILC metastasis is a liver ILC metastasis.

Exemplary Embodiment 79: The method of any one of embodiments 74-76, wherein the ILC metastasis is a female reproductive system ILC metastasis.

Exemplary Embodiment 80: The method of any one of embodiments 74-76, wherein the ILC metastasis is a bone ILC metastasis.

Exemplary Embodiment 81: The method of any one of embodiments 74-76, wherein the ILC metastasis is a skin ILC metastasis.

Exemplary Embodiment 82: The method of any one of embodiments 37-46, wherein the alteration is an alteration in RB1.

Exemplary Embodiment 83: The method of embodiment 82, wherein the alteration comprises one or more of a substitution of one or more nucleotides, an insertion of one or more nucleotides, or a deletion of one or more nucleotides.

Exemplary Embodiment 84: The method of embodiment 82 or embodiment 83, wherein the anti-cancer agent is a SOX2 inhibitor, an EZH2 inhibitor, a chemotherapy, a checkpoint kinase (CHK) inhibitor, a CDC25 phosphatase inhibitor, a polo-like kinase (PLK) inhibitor, or an aurora kinase (AURK) inhibitor.

Exemplary Embodiment 85: The method of any one of embodiments 82-84, wherein the ILC metastasis is a gastrointestinal ILC metastasis.

Exemplary Embodiment 86: The method of any one of embodiments 82-84, wherein the ILC metastasis is a liver ILC metastasis.

Exemplary Embodiment 87: The method of any one of embodiments 82-84, wherein the ILC metastasis is a skin ILC metastasis.

Exemplary Embodiment 88: The method of any one of embodiments 82-84, wherein the ILC metastasis is a bone ILC metastasis.

Exemplary Embodiment 89: The method of any one of embodiments 37-46, wherein the alteration is an alteration in KRAS.

Exemplary Embodiment 90: The method of embodiment 89, wherein the alteration comprises one or more of a substitution of one or more nucleotides, an insertion of one or more nucleotides, or a deletion of one or more nucleotides.

Exemplary Embodiment 91: The method of embodiment 90, wherein the alteration results in one or more amino acid substitutions in a polypeptide encoded by the KRAS gene at amino acid position G12, G13, L19, Q61, A146, K147, F156, or any combination thereof, wherein the numbering of the residues is according to the amino acid sequence of SEQ ID NO: 33.

Exemplary Embodiment 92: The method of embodiment 91, wherein the one or more amino acid substitutions comprise a G12V, G12D, G12A, G12R, G12S, G12C, G12L, G13D, L19F, Q61H, Q61K, Q61E, A146T, K147N, or F156L amino acid substitution, or any combination thereof, wherein the numbering of the residues is according to the amino acid sequence of SEQ ID NO: 33.

Exemplary Embodiment 93: The method of any one of embodiments 89-92, wherein the anti-cancer agent is a kinase inhibitor, a farnesyltransferase inhibitor, a geranylgeranyltransferase inhibitor, a palmitoylation inhibitor, an inhibitor of methylation cleavage, a Raf inhibitor, a MEK inhibitor, an mTOR inhibitor, or an agent that inhibits the modification or post-translational processing of KRAS.

Exemplary Embodiment 94: The method of any one of embodiments 89-93, wherein the ILC metastasis is a gastrointestinal ILC metastasis.

Exemplary Embodiment 95: The method of any one of embodiments 89-93, wherein the ILC metastasis is a liver ILC metastasis.

Exemplary Embodiment 96: The method of any one of embodiments 89-93, wherein the ILC metastasis is a bone ILC metastasis.

Exemplary Embodiment 97: The method of any one of embodiments 37-46, wherein the alteration is an alteration in ERBB2.

Exemplary Embodiment 98: The method of embodiment 97, wherein the alteration comprises one or more of a substitution of one or more nucleotides, an insertion of one or more nucleotides, or a deletion of one or more nucleotides.

Exemplary Embodiment 99: The method of embodiment 98, wherein the alteration results in one or more amino acid substitutions in a polypeptide encoded by the ERBB2 gene at amino acid position S310, S653, V659, R678, V697, E717, T733, L755, I767, D769, G776, V777, T798, V842, L869, or any combination thereof, wherein the numbering of the residues is according to the amino acid sequence of SEQ ID NO: 34.

Exemplary Embodiment 100: The method of embodiment 99, wherein the one or more amino acid substitutions comprise a S310F, S310Y, S653C, V659D, R678Q, V697L, E717K, T733I, L755S, L755P, I767M, D769N, D769Y, D769H, G776V, V777L, T798I, V842I, or L869R amino acid substitution, or any combination thereof, wherein the numbering of the residues is according to the amino acid sequence of SEQ ID NO: 34.

Exemplary Embodiment 101: The method of embodiment 98, wherein the alteration results in a deletion of amino acids L755-T759 (L755_T759del) and/or amino acids L755-E757 (L755_E757del) in a polypeptide encoded by the ERBB2 gene, wherein the numbering of the residues is according to the amino acid sequence of SEQ ID NO: 34.

Exemplary Embodiment 102: The method of embodiment 98, wherein the alteration results in an insertion of one or more amino acid residues between amino acid residues A775 and G776, and/or between amino acid residues P780 and Y781 in a polypeptide encoded by the ERBB2 gene, wherein the numbering of the residues is according to the amino acid sequence of SEQ ID NO: 34.

Exemplary Embodiment 103: The method of embodiment 102, wherein the alteration results in an insertion of the amino acid sequence YVMA (SEQ ID NO: 51) between amino acid residues A775 and G776 (A775_G776insYVMA), and/or of the amino acid sequence GSP (SEQ ID NO: 52) between amino acid residues P780 and Y781 (P780_Y781insGSP), in a polypeptide encoded by the ERBB2 gene, wherein the numbering of the residues is according to the amino acid sequence of SEQ ID NO: 34.

Exemplary Embodiment 104: The method of any one of embodiments 97-103, wherein the anti-cancer agent is a kinase inhibitor, a small molecule, an antibody or antibody fragment, a cellular immunotherapy, or a pan-ERBB inhibitor.

Exemplary Embodiment 105: The method of embodiment 104, wherein the kinase inhibitor is a multi-specific kinase inhibitor, a reversible HER2 inhibitor, an irreversible HER2 inhibitor, a pan-ERBB inhibitor, a dual HER2 inhibitor, a HER2-specific inhibitor, an EGFR inhibitor, or a dual EGFR/ERBB inhibitor.

Exemplary Embodiment 106: The method of any one of embodiments 97-105, wherein the ILC metastasis is a liver ILC metastasis.

Exemplary Embodiment 107: The method of any one of embodiments 97-105, wherein the ILC metastasis is a bone ILC metastasis.

Exemplary Embodiment 108: The method of any one of embodiments 97-105, wherein the ILC metastasis is a skin ILC metastasis.

Exemplary Embodiment 109: The method of any one of embodiments 37-46, wherein the alteration is an alteration in BRAF.

Exemplary Embodiment 110: The method of embodiment 109, wherein the alteration comprises one or more of a substitution of one or more nucleotides, an insertion of one or more nucleotides, or a deletion of one or more nucleotides.

Exemplary Embodiment 111: The method of embodiment 110, wherein the alteration results in one or more amino acid substitutions in a polypeptide encoded by the BRAF gene at amino acid position D380, G464, G466, S467, G469, L485, L584, E586, D594, V600, or any combination thereof, wherein the numbering of the residues is according to the amino acid sequence of SEQ ID NO: 35.

Exemplary Embodiment 112: The method of embodiment 111, wherein the one or more amino acid substitutions comprise a D380H, G464R, G466E, S467L, G469A, G469E, G469R, L485F, L584F, E586K, D594N, D594G, or V600E amino acid substitution, or any combination thereof, wherein the numbering of the residues is according to the amino acid sequence of SEQ ID NO: 35.

Exemplary Embodiment 113: The method of any one of embodiments 109-112, wherein the anti-cancer agent is a kinase inhibitor.

Exemplary Embodiment 114: The method of any one of embodiments 109-113, wherein the ILC metastasis is a bone ILC metastasis.

Exemplary Embodiment 115: The method of any one of embodiments 37-46, wherein the alteration is an alteration in ARID1A.

Exemplary Embodiment 116: The method of embodiment 115, wherein the alteration comprises one or more of a substitution of one or more nucleotides, an insertion of one or more nucleotides, or a deletion of one or more nucleotides.

Exemplary Embodiment 117: The method of embodiment 115 or embodiment 116, wherein the anti-cancer agent is a PARP inhibitor, a bromodomain-containing protein 4 (BRD4) inhibitor, an HDAC inhibitor, a PI3K inhibitor, an ATR inhibitor, an EZH2 inhibitor, an AKT inhibitor, a TrxR inhibitor, a GSH inhibitor, or an immune checkpoint inhibitor.

Exemplary Embodiment 118: The method of any one of embodiments 115-117, wherein the ILC metastasis is a gastrointestinal ILC metastasis.

Exemplary Embodiment 119: The method of any one of embodiments 115-117, wherein the ILC metastasis is a liver ILC metastasis.

Exemplary Embodiment 120: The method of any one of embodiments 115-117, wherein the ILC metastasis is a female reproductive system ILC metastasis.

Exemplary Embodiment 121: The method of any one of embodiments 115-117, wherein the ILC metastasis is a bone ILC metastasis.

Exemplary Embodiment 122: The method of any one of embodiments 115-117, wherein the ILC metastasis is a skin ILC metastasis.

Exemplary Embodiment 123: The method of any one of embodiments 37-46, wherein the alteration is an alteration in PTEN.

Exemplary Embodiment 124: The method of embodiment 123, wherein the alteration is a PTEN deletion.

Exemplary Embodiment 125: The method of embodiment 123 or embodiment 124, wherein the anti-cancer agent is a PI3K inhibitor, an AKT inhibitor, an mTOR inhibitor, or a MET inhibitor.

Exemplary Embodiment 126: The method of any one of embodiments 123-125, wherein the ILC metastasis is a skin ILC metastasis.

Exemplary Embodiment 127: The method of any one of embodiments 123-125, wherein the ILC metastasis is a liver ILC metastasis.

Exemplary Embodiment 128: The method of any one of embodiments 123-125, wherein the ILC metastasis is a female reproductive system ILC metastasis.

Exemplary Embodiment 129: The method of any one of embodiments 37-46, wherein the alteration is an alteration in FGFR2.

Exemplary Embodiment 130: The method of embodiment 129, wherein the alteration comprises one or more of a substitution of one or more nucleotides, an insertion of one or more nucleotides, or a deletion of one or more nucleotides.

Exemplary Embodiment 131: The method of embodiment 130, wherein the alteration results in one or more amino acid substitutions in a polypeptide encoded by the FGFR2 gene at amino acid position S252, P253, Y375, C382, M391, V395, M537, N549, K659, R664, or any combination thereof, wherein the numbering of the residues is according to the amino acid sequence of SEQ ID NO: 38.

Exemplary Embodiment 132: The method of embodiment 131, wherein the one or more amino acid substitutions comprise a S252W, P253R, Y375C, C382R, M391R, V395D, M537I, N549D, N549K, K659M, K659E, K659N, or R664W amino acid substitution, or any combination thereof, wherein the numbering of the residues is according to the amino acid sequence of SEQ ID NO: 38.

Exemplary Embodiment 133: The method of embodiment 130, wherein the alteration results in a frameshift of S799fs*22, wherein the numbering of the residues is according to the amino acid sequence of SEQ ID NO: 38.

Exemplary Embodiment 134: The method of any one of embodiments 129-133, wherein the anti-cancer agent is a kinase inhibitor.

Exemplary Embodiment 135: The method of any one of embodiments 129-134, wherein the ILC metastasis is a liver ILC metastasis.

Exemplary Embodiment 136: The method of any one of embodiments 129-134, wherein the ILC metastasis is a bone ILC metastasis.

Exemplary Embodiment 137: The method of any one of embodiments 37-46, wherein the alteration is an alteration in SMAD4.

Exemplary Embodiment 138: The method of embodiment 137, wherein the anti-cancer agent is a PARP inhibitor.

Exemplary Embodiment 139: The method of embodiment 137 or embodiment 138, wherein the ILC metastasis is a liver ILC metastasis.

Exemplary Embodiment 140: The method of any one of embodiments 37-46, wherein the alteration is an alteration in PTPN11.

Exemplary Embodiment 141: The method of embodiment 140, wherein the alteration comprises one or more of a substitution of one or more nucleotides, an insertion of one or more nucleotides, or a deletion of one or more nucleotides.

Exemplary Embodiment 142: The method of embodiment 140 or embodiment 141, wherein the anti-cancer agent is a small molecule inhibitor or a kinase inhibitor.

Exemplary Embodiment 143: The method of any one of embodiments 37-46, wherein the alteration is an alteration in TERT.

Exemplary Embodiment 144: The method of embodiment 143, wherein the alteration comprises one or more of a substitution of one or more nucleotides, an insertion of one or more nucleotides, or a deletion of one or more nucleotides.

Exemplary Embodiment 145: The method of embodiment 143 or embodiment 144, wherein the alteration is an alteration in the promoter of TERT, wherein the alteration in the promoter of TERT comprises a −146C>T, −139_−138CC>TT, or −124C>T mutation, or any combination thereof, wherein the numbering of the nucleotides is according to SEQ ID NO: 22.

Exemplary Embodiment 146: The method of any one of embodiments 143-145, wherein the anti-cancer agent is a small molecule inhibitor, a modified nucleotide or nucleoside analog, a G-quadruplex stabilizer, a heat shock protein (HSP) inhibitor, or a MYC inhibitor.

Exemplary Embodiment 147: The method of any one of embodiments 143-146, wherein the ILC metastasis is a gastrointestinal ILC metastasis.

Exemplary Embodiment 148: The method of any one of embodiments 37-46, wherein the alteration is an alteration in ALK.

Exemplary Embodiment 149: The method of embodiment 148, wherein the alteration comprises one or more of a substitution of one or more nucleotides, an insertion of one or more nucleotides, or a deletion of one or more nucleotides.

Exemplary Embodiment 150: The method of embodiment 148 or embodiment 149, wherein the anti-cancer agent is a kinase inhibitor, a heat shock protein (HSP) inhibitor or a MYC inhibitor.

Exemplary Embodiment 151: The method of any one of embodiments 37-46, wherein the alteration is an alteration in NCOR1.

Exemplary Embodiment 152: The method of embodiment 151, wherein the alteration comprises one or more of a substitution of one or more nucleotides, an insertion of one or more nucleotides, or a deletion of one or more nucleotides.

Exemplary Embodiment 153: The method of embodiment 151 or embodiment 152, wherein the ILC metastasis is a female reproductive system ILC metastasis.

Exemplary Embodiment 154: The method of any one of embodiments 37-46, wherein the alteration is an alteration in APC.

Exemplary Embodiment 155: The method of embodiment 154, wherein the alteration comprises one or more of a substitution of one or more nucleotides, an insertion of one or more nucleotides, or a deletion of one or more nucleotides.

Exemplary Embodiment 156: The method of embodiment 154 or embodiment 155, wherein the anti-cancer agent is a beta-catenin inhibitor or an APC inhibitor.

Exemplary Embodiment 157: The method of any one of embodiments 1-156, further comprising administering an additional anti-cancer therapy to the individual.

Exemplary Embodiment 158: The method of embodiment 157, wherein the additional anti-cancer therapy is a surgery, a radiotherapy, a chemotherapy, an anti-angiogenic therapy, an anti-DNA repair therapy, an immunotherapy, an anti-neoplastic agent, a cytotoxic agent, an anti-inflammatory therapy, or any combination thereof.

Exemplary Embodiment 159: The method of any one of embodiments 1-158, wherein the ILC metastasis comprises one or more deleterious CDH1 mutations.

Exemplary Embodiment 160: The method of any one of embodiments 1-159, further comprising acquiring knowledge of one or more deleterious CDH1 mutations in a sample from the individual.

Exemplary Embodiment 161: The method of embodiment 160, wherein the acquiring knowledge comprises detecting the one or more deleterious CDH1 mutations in a sample from the individual.

Exemplary Embodiment 162: The method of any one of embodiments 159-161, wherein the one or more deleterious CDH1 mutations comprise one or more of a substitution of one or more nucleotides, an insertion of one or more nucleotides, or a deletion of one or more nucleotides in a gene encoding a CDH1 polypeptide.

Exemplary Embodiment 163: The method of any one of embodiments 159-162, wherein the one or more deleterious CDH1 mutations result in loss of function of a CDH1 polypeptide.

Exemplary Embodiment 164: The method of any one of embodiments 159-163, wherein the one or more deleterious CDH1 mutations result in a mutation of the CDH1 start codon.

Exemplary Embodiment 165: The method of any one of embodiments 159-163, wherein the one or more deleterious CDH1 mutations result in one or more amino acid substitutions in a CDH1 polypeptide at amino acid position E243, D402, D433, A634, or any combination thereof, wherein the numbering of the residues is according to the amino acid sequence of SEQ ID NO: 50.

Exemplary Embodiment 166: The method of embodiment 165, wherein the one or more amino acid substitutions comprise a E243K, D402N, D433N, or A634V amino acid substitution, or any combination thereof, wherein the numbering of the residues is according to the amino acid sequence of SEQ ID NO: 50.

Exemplary Embodiment 167: The method of any one of embodiments 159-163, wherein the one or more deleterious CDH1 mutations result in a stop codon at amino acid position W4 (W4*), S9 (S9*), Q16 (Q16*), W20 (W20*), Q23 (Q23*), E24 (E24*), E26 (E26*), E35 (E35*), Y37 (Y37*), E47 (E47*), R54 (R54*), E58 (E58*), R63 (R63*), Q64 (Q64*), R74 (R74*), W103 (W103*), Q129 (Q129*), R150 (R150*), Q152 (Q152*), Q177 (Q177*), Y190 (Y190*), Y228 (Y228*), E243 (E243*), Q255 (Q255*), Q264 (Q264*), Y302 (Y302*), R335 (R335*), Q346 (Q346*), E353 (E353*), Q383 (Q383*), Q449 (Q449*), E463 (E463*), Y523 (Y523*), W526 (W526*), R598 (R598*), Q610 (Q610*), Q641 (Q641*), E648 (E648*), Q699 (Q699*), Q706 (Q706*), Q765 (Q765*), Q771 (Q771*), E806 (E806*), Y827 (Y827*), or any combination thereof, wherein the numbering of the residues is according to the amino acid sequence of SEQ ID NO: 50.

Exemplary Embodiment 168: The method of any one of embodiments 159-163, wherein the one or more deleterious CDH1 mutations comprise a frameshift, wherein the frameshift is an T115fs*53, P127fs*41, P200fs*6, P200fs*16, V202fs*7, H233fs*11, P277fs*5, Y302fs*1, S337fs*12, Y523fs*1, L585fs*4, I650fs*3, 1650fs*13, A719fs*29, or Q765fs*4 frameshift, wherein the numbering of the residues is according to the amino acid sequence of SEQ ID NO: 50.

Exemplary Embodiment 169: The method of any one of embodiments 159-163, wherein the one or more deleterious CDH1 mutations comprise a splice site mutation, wherein the splice site mutation is a 48+1G>A, 1565+1G>A, or 1565+1G>T splice site mutation, wherein the numbering of the nucleotides is according to SEQ ID NO: 49.

Exemplary Embodiment 170: The method of any one of embodiments 37-169, wherein the sample from the individual comprises fluid, cells, or tissue.

Exemplary Embodiment 171: The method of embodiment 170, wherein the sample from the individual comprises a tumor biopsy or a circulating tumor cell.

Exemplary Embodiment 172: The method of any one of embodiments 37-171, wherein the sample from the individual comprises nucleic acids.

Exemplary Embodiment 173: The method of embodiment 172, wherein the sample from the individual comprises mRNA, genomic DNA, circulating tumor DNA, cell-free DNA, or cell-free RNA.

Exemplary Embodiment 174: The method of any one of embodiments 38-173, wherein the alteration is detected in the sample by one or more methods selected from the group consisting of a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), and mass-spectrometric genotyping.

Exemplary Embodiment 175: The method of any one of embodiments 37-171, wherein the sample from the individual comprises one or more proteins.

Exemplary Embodiment 176: The method of any one of embodiments 37-171 and 175, wherein the acquiring knowledge comprises detecting the alteration in a polypeptide encoded by the one or more genes in the sample from the individual.

Exemplary Embodiment 177: The method of embodiment 176, wherein the alteration is detected in the sample by one or more methods selected from the group consisting of immunoblotting, enzyme linked immunosorbent assay (ELISA), immunohistochemistry, and mass spectrometry.

Exemplary Embodiment 178: A method for genomic profiling of an invasive lobular carcinoma (ILC) metastasis, comprising: (a) detecting one or more biomarkers in a sample from an individual having an ILC metastasis, wherein the one or more biomarkers are selected from the group consisting of: (i) a tumor mutational burden (TMB) of at least about 10 mutations/megabase (mut/Mb), (ii) a PD-L1-positive ILC metastasis, and (iii) an alteration in one or more genes, wherein the one or more genes are selected from the group consisting of: PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, and KMT2D; and (b) providing a report to a party.

Exemplary Embodiment 179: The method of embodiment 178, wherein the individual is a human.

Exemplary Embodiment 180: The method of embodiment 178 or embodiment 179, wherein the ILC metastasis is selected from the group consisting of a bone ILC metastasis, a female reproductive system ILC metastasis, a gastrointestinal ILC metastasis, a liver ILC metastasis, and a skin ILC metastasis.

Exemplary Embodiment 181: The method of any one of embodiments 178-180, wherein the ILC metastasis is a bone ILC metastasis.

Exemplary Embodiment 182: The method of any one of embodiments 178-180, wherein the ILC metastasis is a female reproductive system ILC metastasis.

Exemplary Embodiment 183: The method of any one of embodiments 178-180, wherein the ILC metastasis is a gastrointestinal ILC metastasis.

Exemplary Embodiment 184: The method of any one of embodiments 178-180, wherein the ILC metastasis is a liver ILC metastasis.

Exemplary Embodiment 185: The method of any one of embodiments 178-180, wherein the ILC metastasis is a skin ILC metastasis.

Exemplary Embodiment 186: The method of any one of embodiments 178-185, wherein the ILC metastasis comprises one or more deleterious CDH1 mutations.

Exemplary Embodiment 187: The method of any one of embodiments 178-186, further comprising acquiring knowledge of one or more deleterious CDH1 mutations in a sample from the individual.

Exemplary Embodiment 188: The method of embodiment 187, wherein the acquiring knowledge comprises detecting the one or more deleterious CDH1 mutations in a sample from the individual.

Exemplary Embodiment 189: The method of any one of embodiments 186-188, wherein the one or more deleterious CDH1 mutations comprise one or more of a substitution of one or more nucleotides, an insertion of one or more nucleotides, or a deletion of one or more nucleotides in a gene encoding a CDH1 polypeptide.

Exemplary Embodiment 190: The method of any one of embodiments 186-189, wherein the one or more deleterious CDH1 mutations result in loss of function of a CDH1 polypeptide.

Exemplary Embodiment 191: The method of any one of embodiments 186-190, wherein the one or more deleterious CDH1 mutations result in a mutation of the CDH1 start codon.

Exemplary Embodiment 192: The method of any one of embodiments 186-190, wherein the one or more deleterious CDH1 mutations result in one or more amino acid substitutions in a CDH1 polypeptide at amino acid position E243, D402, D433, A634, or any combination thereof, wherein the numbering of the residues is according to the amino acid sequence of SEQ ID NO: 50.

Exemplary Embodiment 193: The method of embodiment 192, wherein the one or more amino acid substitutions comprise a E243K, D402N, D433N, or A634V amino acid substitution, or any combination thereof, wherein the numbering of the residues is according to the amino acid sequence of SEQ ID NO: 50.

Exemplary Embodiment 194: The method of any one of embodiments 186-190, wherein the one or more deleterious CDH1 mutations result in a stop codon at amino acid position W4 (W4*), S9 (S9*), Q16 (Q16*), W20 (W20*), Q23 (Q23*), E24 (E24*), E26 (E26*), E35 (E35*), Y37 (Y37*), E47 (E47*), R54 (R54*), E58 (E58*), R63 (R63*), Q64 (Q64*), R74 (R74*), W103 (W103*), Q129 (Q129*), R150 (R150*), Q152 (Q152*), Q177 (Q177*), Y190 (Y190*), Y228 (Y228*), E243 (E243*), Q255 (Q255*), Q264 (Q264*), Y302 (Y302*), R335 (R335*), Q346 (Q346*), E353 (E353*), Q383 (Q383*), Q449 (Q449*), E463 (E463*), Y523 (Y523*), W526 (W526*), R598 (R598*), Q610 (Q610*), Q641 (Q641*), E648 (E648*), Q699 (Q699*), Q706 (Q706*), Q765 (Q765*), Q771 (Q771*), E806 (E806*), Y827 (Y827*), or any combination thereof, wherein the numbering of the residues is according to the amino acid sequence of SEQ ID NO: 50.

Exemplary Embodiment 195: The method of any one of embodiments 186-190, wherein the one or more deleterious CDH1 mutations comprise a frameshift, wherein the frameshift is a T115fs*53, P127fs*41, P200fs*6, P200fs*16, V202fs*7, H233fs*11, P277fs*5, Y302fs*1, S337fs*12, Y523fs*1, L585fs*4, I650fs*3, I650fs*13, A719fs*29, or Q765fs*4 frameshift, wherein the numbering of the residues is according to the amino acid sequence of SEQ ID NO: 50.

Exemplary Embodiment 196: The method of any one of embodiments 186-190, wherein the one or more deleterious CDH1 mutations comprise a splice site mutation, wherein the splice site mutation is a 48+1G>A, 1565+1G>A, or 1565+1G>T splice site mutation, wherein the numbering of the nucleotides is according to SEQ ID NO: 49.

Exemplary Embodiment 197: The method of any one of embodiments 178-196, wherein the sample from the individual comprises fluid, cells, or tissue.

Exemplary Embodiment 198: The method of embodiment 197, wherein the sample from the individual comprises a tumor biopsy or a circulating tumor cell.

Exemplary Embodiment 199: The method of any one of embodiments 178-198, wherein the sample from the individual comprises nucleic acids.

Exemplary Embodiment 200: The method of embodiment 199, wherein the sample from the individual comprises mRNA, genomic DNA, circulating tumor DNA, cell-free DNA, or cell-free RNA.

Exemplary Embodiment 201: The method of any one of embodiments 178-200, wherein detecting a TMB of at least about 10 mut/Mb comprises measuring the level of TMB in the sample from the individual.

Exemplary Embodiment 202: The method of embodiment 201, wherein TMB is measured on between about 0.8 Mb and about 1.1 Mb.

Exemplary Embodiment 203: The method of embodiment 201 or embodiment 202, wherein TMB is measured in the sample by whole exome sequencing, whole genome sequencing, or gene-targeted sequencing.

Exemplary Embodiment 204: The method of any one of embodiments 178-203, comprising detecting a TMB of at least about 10 mut/Mb in a sample from an individual having a gastrointestinal ILC metastasis.

Exemplary Embodiment 205: The method of any one of embodiments 178-203, comprising detecting a TMB of at least about 10 mut/Mb in a sample from an individual having a liver ILC metastasis.

Exemplary Embodiment 206: The method of any one of embodiments 178-203, comprising detecting a TMB of at least about 10 mut/Mb in a sample from an individual having a female reproductive system ILC metastasis.

Exemplary Embodiment 207: The method of any one of embodiments 178-203, comprising detecting a TMB of at least about 10 mut/Mb in a sample from an individual having a bone ILC metastasis.

Exemplary Embodiment 208: The method of any one of embodiments 178-203, comprising detecting a TMB of at least about 10 mut/Mb in a sample from an individual having a skin ILC metastasis.

Exemplary Embodiment 209: The method of any one of embodiments 178-200, wherein detecting a PD-L1-positive ILC metastasis comprises measuring the level of PD-L1 expression in the sample from the individual.

Exemplary Embodiment 210: The method of embodiment 209, wherein the level of PD-L1 expression is measured using an immunohistochemistry assay.

Exemplary Embodiment 211: The method of embodiment 209 or embodiment 210, wherein the level of PD-L1 expression is determined based on PD-L1 expression in tumor infiltrating immune cells (ICs) and/or tumor cells (TCs).

Exemplary Embodiment 212: The method of any one of embodiments 209-211, wherein a PD-L1-positive ILC metastasis is detected if at least about 1% of ICs in the sample are PD-L1-positive.

Exemplary Embodiment 213: The method of any one of embodiments 178-200 and 209-212, comprising detecting a PD-L1-positive ILC metastasis in a sample from an individual having a gastrointestinal ILC metastasis.

Exemplary Embodiment 214: The method of any one of embodiments 178-200 and 209-212, comprising detecting a PD-L1-positive ILC metastasis in a sample from an individual having a liver ILC metastasis.

Exemplary Embodiment 215: The method of any one of embodiments 178-200 and 209-212, comprising detecting a PD-L1-positive ILC metastasis in a sample from an individual having a female reproductive system ILC metastasis.

Exemplary Embodiment 216: The method of any one of embodiments 178-200 and 209-212, comprising detecting a PD-L1-positive ILC metastasis in a sample from an individual having a skin ILC metastasis.

Exemplary Embodiment 217: The method of any one of embodiments 178-200, comprising detecting: (a) one or more of a substitution of one or more nucleotides, an insertion of one or more nucleotides, or a deletion of one or more nucleotides in one or more genes selected from the group consisting of PIK3CA, ESR1, NF1, RB1, ERBB2, ARID1A, NCOR1, FOXP1, APC, CASP8, PTPN11, TERT, FGFR2, KRAS, BRAF, SMAD4, SOX9 and ALK; (b) a PTEN deletion; or (c) a KMT2D rearrangement.

Exemplary Embodiment 218: The method of any one of embodiments 178-200, comprising detecting an alteration in BRCA1 and/or BRCA2 in a sample from an individual having a female reproductive system ILC metastasis.

Exemplary Embodiment 219: The method of any one of embodiments 178-200, comprising detecting an alteration in BRCA1 and/or BRCA2 in a sample from an individual having a bone ILC metastasis.

Exemplary Embodiment 220: The method of any one of embodiments 178-200, comprising detecting an alteration in BRCA1 and/or BRCA2 in a sample from an individual having a skin ILC metastasis.

Exemplary Embodiment 221: The method of any one of embodiments 178-200, comprising detecting an alteration in BRCA1 and/or BRCA2 in a sample from an individual having a liver ILC metastasis.

Exemplary Embodiment 222: The method of any one of embodiments 178-200 and 217, comprising detecting an alteration in PIK3CA in a sample from an individual having a gastrointestinal ILC metastasis.

Exemplary Embodiment 223: The method of any one of embodiments 178-200 and 217, comprising detecting an alteration in PIK3CA in a sample from an individual having a liver ILC

Exemplary Embodiment 224: The method of any one of embodiments 178-200 and 217, comprising detecting an alteration in PIK3CA in a sample from an individual having a female reproductive system ILC metastasis.

Exemplary Embodiment 225: The method of any one of embodiments 178-200 and 217, comprising detecting an alteration in PIK3CA in a sample from an individual having a bone ILC metastasis.

Exemplary Embodiment 226: The method of any one of embodiments 178-200 and 217, comprising detecting an alteration in PIK3CA in a sample from an individual having a skin ILC metastasis.

Exemplary Embodiment 227: The method of any one of embodiments 222-226, wherein the alteration results in one or more amino acid substitutions in a polypeptide encoded by the PIK3CA gene at amino acid position E81, R108, K111, G118, N345, D350, E365, E418, C420, E453, P539, E542, E545, Q546, E726, E970, M1004, M1043, N1044, H1047, G1049, or any combination thereof, wherein the numbering of the residues is according to the amino acid sequence of SEQ ID NO: 26.

Exemplary Embodiment 228: The method of embodiment 227, wherein the one or more amino acid substitutions comprise a E81K, R108H, K111N, G118D, N345K, D350N, E365K, E418K, C420R, E453K, E453Q, P539R, E542K, E545K, E545A, E545Q, Q546R, Q546K, E726K, E970K, M1004I, M1043I, N1044K, H1047R, H1047L, or G1049R amino acid substitution, or any combination thereof, wherein the numbering of the residues is according to the amino acid sequence of SEQ ID NO: 26.

Exemplary Embodiment 229: The method of any one of embodiments 222-226, wherein the alteration results in a deletion of amino acid residue E110 (E110del) in a polypeptide encoded by the PIK3CA gene, wherein the numbering of the residues is according to the amino acid sequence of SEQ ID NO: 26.

Exemplary Embodiment 230: The method of any one of embodiments 178-200 and 217, comprising detecting an alteration in ESR1 in a sample from an individual having a gastrointestinal ILC metastasis.

Exemplary Embodiment 231: The method of any one of embodiments 178-200 and 217, comprising detecting an alteration in ESR1 in a sample from an individual having a liver ILC metastasis.

Exemplary Embodiment 232: The method of any one of embodiments 178-200 and 217, comprising detecting an alteration in ESR1 in a sample from an individual having a bone ILC

Exemplary Embodiment 233: The method of any one of embodiments 178-200 and 217, comprising detecting an alteration in ESR1 in a sample from an individual having a skin ILC

Exemplary Embodiment 234: The method of any one of embodiments 230-233, wherein the alteration results in one or more amino acid substitutions in a polypeptide encoded by the ESR1 gene at amino acid position E380, V418, S463, V533, L536, Y537, D538, or any combination thereof, wherein the numbering of the residues is according to the amino acid sequence of SEQ ID NO: 29.

Exemplary Embodiment 235: The method of embodiment 234, wherein the one or more amino acid substitutions comprise a E380Q, V418E, S463P, V533M, L536Q, L536H, L536P, L536R, Y537S, Y537N, Y537C, Y537D, or D538G amino acid substitution, or any combination thereof, wherein the numbering of the residues is according to the amino acid sequence of SEQ ID NO: 29.

Exemplary Embodiment 236: The method of any one of embodiments 230-233, wherein the alteration results in a deletion of amino acid V422 (V422del) and/or a deletion of amino acids V533-L536 (V533_L536del) in a polypeptide encoded by the ESR1 gene, wherein the numbering of the residues is according to the amino acid sequence of SEQ ID NO: 29.

Exemplary Embodiment 237: The method of any one of embodiments 178-200 and 217, comprising detecting an alteration in ERBB2 in a sample from an individual having a liver ILC metastasis.

Exemplary Embodiment 238: The method of any one of embodiments 178-200 and 217, comprising detecting an alteration in ERBB2 in a sample from an individual having a bone ILC metastasis.

Exemplary Embodiment 239: The method of any one of embodiments 178-200 and 217, comprising detecting an alteration in ERBB2 in a sample from an individual having a skin ILC metastasis.

Exemplary Embodiment 240: The method of any one of embodiments 237-239, wherein the alteration results in one or more amino acid substitutions in a polypeptide encoded by the ERBB2 gene at amino acid position S310, S653, V659, R678, V697, E717, T733, L755, I767, D769, G776, V777, T798, V842, L869, or any combination thereof, wherein the numbering of the residues is according to the amino acid sequence of SEQ ID NO: 34.

Exemplary Embodiment 241: The method of embodiment 240, wherein the one or more amino acid substitutions comprise a S310F, S310Y, S653C, V659D, R678Q, V697L, E717K, T733I, L755S, L755P, I767M, D769N, D769Y, D769H, G776V, V777L, T798I, V842I, or L869R amino acid substitution, or any combination thereof, wherein the numbering of the residues is according to the amino acid sequence of SEQ ID NO: 34.

Exemplary Embodiment 242: The method of any one of embodiments 237-239, wherein the alteration results in a deletion of amino acids L755-T759 (L755_T759del) and/or amino acids L755-E757 (L755_E757del) in a polypeptide encoded by the ERBB2 gene, wherein the numbering of the residues is according to the amino acid sequence of SEQ ID NO: 34.

Exemplary Embodiment 243: The method of any one of embodiments 237-239, wherein the alteration results in an insertion of one or more amino acid residues between amino acid residues A775 and G776, and/or between amino acid residues P780 and Y781 in a polypeptide encoded by the ERBB2 gene, wherein the numbering of the residues is according to the amino acid sequence of SEQ ID NO: 34.

Exemplary Embodiment 244: The method of embodiment 243, wherein the alteration results in an insertion of the amino acid sequence YVMA (SEQ ID NO: 51) between amino acid residues A775 and G776 (A775_G776insYVMA), and/or of the amino acid sequence GSP (SEQ ID NO: 52) between amino acid residues P780 and Y781 (P780_Y781insGSP), in a polypeptide encoded by the ERBB2 gene, wherein the numbering of the residues is according to the amino acid sequence of SEQ ID NO: 34.

Exemplary Embodiment 245: The method of any one of embodiments 178-200 and 217, comprising detecting an alteration in ARID1A in a sample from an individual having a gastrointestinal ILC metastasis.

Exemplary Embodiment 246: The method of any one of embodiments 178-200 and 217, comprising detecting an alteration in ARID1A in a sample from an individual having a liver ILC metastasis.

Exemplary Embodiment 247: The method of any one of embodiments 178-200 and 217, comprising detecting an alteration in ARID1A in a sample from an individual having a female reproductive system ILC metastasis.

Exemplary Embodiment 248: The method of any one of embodiments 178-200 and 217, comprising detecting an alteration in ARID1A in a sample from an individual having a bone ILC metastasis.

Exemplary Embodiment 249: The method of any one of embodiments 178-200 and 217, comprising detecting an alteration in ARID1A in a sample from an individual having a skin ILC metastasis.

Exemplary Embodiment 250: The method of any one of embodiments 178-200 and 217, comprising detecting an alteration in NF1 in a sample from an individual having a gastrointestinal ILC metastasis.

Exemplary Embodiment 251: The method of any one of embodiments 178-200 and 217, comprising detecting an alteration in NF1 in a sample from an individual having a liver ILC metastasis.

Exemplary Embodiment 252: The method of any one of embodiments 178-200 and 217, comprising detecting an alteration in NF1 in a sample from an individual having a female reproductive system ILC metastasis.

Exemplary Embodiment 253: The method of any one of embodiments 178-200 and 217, comprising detecting an alteration in NF1 in a sample from an individual having a bone ILC

Exemplary Embodiment 254: The method of any one of embodiments 178-200 and 217, comprising detecting an alteration in NF1 in a sample from an individual having a skin ILC metastasis.

Exemplary Embodiment 255: The method of any one of embodiments 178-200 and 217, comprising detecting an alteration in RB1 in a sample from an individual having a gastrointestinal ILC metastasis.

Exemplary Embodiment 256: The method of any one of embodiments 178-200 and 217, comprising detecting an alteration in RB1 in a sample from an individual having a liver ILC metastasis.

Exemplary Embodiment 257: The method of any one of embodiments 178-200 and 217, comprising detecting an alteration in RB1 in a sample from an individual having a skin ILC metastasis.

Exemplary Embodiment 258: The method of any one of embodiments 178-200 and 217, comprising detecting an alteration in RB1 in a sample from an individual having a bone ILC metastasis.

Exemplary Embodiment 259: The method of any one of embodiments 178-200 and 217, comprising detecting an alteration in KRAS in a sample from an individual having a gastrointestinal ILC metastasis.

Exemplary Embodiment 260: The method of any one of embodiments 178-200 and 217, comprising detecting an alteration in KRAS in a sample from an individual having a liver ILC metastasis.

Exemplary Embodiment 261: The method of any one of embodiments 178-200 and 217, comprising detecting an alteration in KRAS in a sample from an individual having a bone ILC metastasis.

Exemplary Embodiment 262: The method of any one of embodiments 259-261, wherein the alteration results in one or more amino acid substitutions in a polypeptide encoded by the KRAS gene at amino acid position G12, G13, L19, Q61, A146, K147, F156, or any combination thereof, wherein the numbering of the residues is according to the amino acid sequence of SEQ ID NO: 33.

Exemplary Embodiment 263: The method of embodiment 262, wherein the one or more amino acid substitutions comprise a G12V, G12D, G12A, G12R, G12S, G12C, G12L, G13D, L19F, Q61H, Q61K, Q61E, A146T, K147N, or F156L amino acid substitution, or any combination thereof, wherein the numbering of the residues is according to the amino acid sequence of SEQ ID NO: 33.

Exemplary Embodiment 264: The method of any one of embodiments 178-200 and 217, comprising detecting an alteration in PTEN in a sample from an individual having a skin ILC

Exemplary Embodiment 265: The method of any one of embodiments 178-200 and 217, comprising detecting an alteration in PTEN in a sample from an individual having a liver ILC metastasis.

Exemplary Embodiment 266: The method of any one of embodiments 178-200 and 217, comprising detecting an alteration in PTEN in a sample from an individual having a female reproductive system ILC metastasis.

Exemplary Embodiment 267: The method of any one of embodiments 178-200 and 217, comprising detecting an alteration in NCOR1 in a sample from an individual having a female reproductive system ILC metastasis.

Exemplary Embodiment 268: The method of any one of embodiments 178-200 and 217, comprising detecting an alteration in BRAF in a sample from an individual having a bone ILC metastasis.

Exemplary Embodiment 269: The method of embodiment 268, wherein the alteration results in one or more amino acid substitutions in a polypeptide encoded by the BRAF gene at amino acid position D380, G464, G466, S467, G469, L485, L584, E586, D594, V600, or any combination thereof, wherein the numbering of the residues is according to the amino acid sequence of SEQ ID NO: 35.

Exemplary Embodiment 270: The method of embodiment 269, wherein the one or more amino acid substitutions comprise a D380H, G464R, G466E, S467L, G469A, G469E, G469R, L485F, L584F, E586K, D594N, D594G, or V600E amino acid substitution, or any combination thereof, wherein the numbering of the residues is according to the amino acid sequence of SEQ ID NO: 35.

Exemplary Embodiment 271: The method of any one of embodiments 178-200 and 217, comprising detecting an alteration in FGFR2 in a sample from an individual having a liver ILC metastasis.

Exemplary Embodiment 272: The method of any one of embodiments 178-200 and 217, comprising detecting an alteration in FGFR2 in a sample from an individual having a bone ILC metastasis.

Exemplary Embodiment 273: The method of embodiment 271 or embodiment 272, wherein the alteration results in one or more amino acid substitutions in a polypeptide encoded by the FGFR2 gene at amino acid position S252, P253, Y375, C382, M391, V395, M537, N549, K659, R664, or any combination thereof, wherein the numbering of the residues is according to the amino acid sequence of SEQ ID NO: 38.

Exemplary Embodiment 274: The method of embodiment 273, wherein the one or more amino acid substitutions comprise a S252W, P253R, Y375C, C382R, M391R, V395D, M537I, N549D, N549K, K659M, K659E, K659N, or R664W amino acid substitution, or any combination thereof, wherein the numbering of the residues is according to the amino acid sequence of SEQ ID NO: 38.

Exemplary Embodiment 275: The method of embodiment 271 or embodiment 272, wherein the alteration results in a frameshift of S799fs*22, wherein the numbering of the residues is according to the amino acid sequence of SEQ ID NO: 38.

Exemplary Embodiment 276: The method of any one of embodiments 178-200 and 217, comprising detecting an alteration in TERT in a sample from an individual having a gastrointestinal ILC metastasis.

Exemplary Embodiment 277: The method of embodiment 276, wherein the alteration is an alteration in the promoter of TERT, wherein the alteration in the promoter of TERT comprises a −146C>T, −139_−138CC>TT, or −124C>T mutation, or any combination thereof, wherein the numbering of the nucleotides is according to SEQ ID NO: 22.

Exemplary Embodiment 278: The method of any one of embodiments 178-200 and 217, comprising detecting an alteration in SMAD4 in a sample from an individual having a liver ILC metastasis.

Exemplary Embodiment 279: The method of any one of embodiments 178-200 and 217-278, wherein the alteration in the one or more genes is detected in the sample by one or more methods selected from the group consisting of a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), and mass-spectrometric genotyping.

Exemplary Embodiment 280: The method of any one of embodiments 178-200 and 217-279, wherein the sample from the individual comprises one or more proteins.

Exemplary Embodiment 281: The method of any one of embodiments 178-200, 217-278, and 280, wherein the alteration in the one or more genes is detected in a polypeptide encoded by the one or more genes.

Exemplary Embodiment 282: The method of embodiment 281, wherein the alteration is detected in the sample by one or more methods selected from the group consisting of immunoblotting, enzyme linked immunosorbent assay (ELISA), immunohistochemistry, and mass spectrometry.

Exemplary Embodiment 283: The method of any one of embodiments 178-282, wherein the party is one or more of the individual, a caregiver, a physician, an oncologist, a hospital, a clinic, a third-party payor, an insurance company, or a government office.

Exemplary Embodiment 284: The method of any one of embodiments 178-283, wherein the report is in electronic, web-based, and/or paper form.

Exemplary Embodiment 285: The method of any one of embodiments 178-284, wherein the report identifies the presence or absence of the one or more biomarkers in a sample from the individual, and optionally comprises an identifier for the individual from which the sample was obtained.

Exemplary Embodiment 286: The method of any one of embodiments 178-285, wherein the report comprises one or more of: (a) information on the role of the one or more biomarkers in disease; (b) information on prognosis, resistance, or potential or suggested therapeutic options; (c) information on the likely effectiveness of a therapeutic option, the acceptability of a therapeutic option, or the advisability of applying the therapeutic option to an individual; or (d) information, or a recommendation on, the administration of a drug.

Exemplary Embodiment 287: The method of any one of embodiments 178-286, further comprising obtaining the sample from the individual.

Exemplary Embodiment 288: A system, comprising: a memory configured to store one or more program instructions; and one or more processors configured to execute the one or more program instructions, the one or more program instructions when executed by the one or more processors are configured to: (a) obtain a plurality of sequence reads of one or more nucleic acids, wherein the one or more nucleic acids are derived from a sample obtained from an individual; (b) analyze the plurality of sequence reads for the presence of a tumor mutational burden (TMB) of at least about 10 mutations/megabase (mut/Mb); and (c) detect, based on the analyzing, a TMB of at least about 10 mut/Mb, in the sample.

Exemplary Embodiment 289: The system of embodiment 288, wherein the sample is a sample from an individual having an invasive lobular carcinoma (ILC) metastasis.

Exemplary Embodiment 290: The system of embodiment 288 or embodiment 289, wherein the analyzing is based on between about 0.8 Mb and about 1.1 Mb of sequenced nucleic acids.

Exemplary Embodiment 291: The system of embodiment 289 or embodiment 290, wherein the ILC metastasis is a bone ILC metastasis, a female reproductive system ILC metastasis, a gastrointestinal ILC metastasis, a liver ILC metastasis, or a skin ILC metastasis.

Exemplary Embodiment 292: The system of any one of embodiments 288-291, wherein the plurality of sequence reads are obtained by whole exome sequencing, whole genome sequencing, or gene-targeted sequencing.

Exemplary Embodiment 293: A non-transitory computer readable storage medium comprising one or more programs executable by one or more computer processors for performing a method, comprising: (a) obtaining, using the one or more processors, a plurality of sequence reads of one or more nucleic acids, wherein the one or more nucleic acids are derived from a sample obtained from an individual; (b) analyzing, using the one or more processors, the plurality of sequence reads for the presence of a tumor mutational burden (TMB) of at least about 10 mutations/megabase (mut/Mb); and (c) detecting, using the one or more processors and based on the analyzing, a TMB of at least about 10 mut/Mb, in the sample.

Exemplary Embodiment 294: The non-transitory computer readable storage medium of embodiment 293, wherein the sample is a sample from an individual having an invasive lobular carcinoma (ILC) metastasis.

Exemplary Embodiment 295: The non-transitory computer readable storage medium of embodiment 293 or embodiment 294, wherein the analyzing is based on between about 0.8 Mb and about 1.1 Mb of sequenced nucleic acids.

Exemplary Embodiment 296: The non-transitory computer readable storage medium of embodiment 294 or embodiment 295, wherein the ILC metastasis is a bone ILC metastasis, a female reproductive system ILC metastasis, a gastrointestinal ILC metastasis, a liver ILC metastasis, or a skin ILC metastasis.

Exemplary Embodiment 297: The non-transitory computer readable storage medium of any one of embodiments 293-296, wherein the plurality of sequence reads are obtained by whole exome sequencing, whole genome sequencing, or gene-targeted sequencing.

Exemplary Embodiment 298: A system, comprising: a memory configured to store one or more program instructions; and one or more processors configured to execute the one or more program instructions, the one or more program instructions when executed by the one or more processors are configured to: (a) obtain a plurality of sequence reads of one or more nucleic acids, wherein the one or more nucleic acids are derived from a sample obtained from an individual; (b) analyze the plurality of sequence reads for the presence of an alteration in one or more genes, wherein the one or more genes comprise PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, CDH1 or KMT2D; and (c) detect, based on the analyzing, an alteration in one or more genes, wherein the one or more genes comprise PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, CDH1 or KMT2D, in the sample.

Exemplary Embodiment 299: The system of embodiment 298, wherein the sample is a sample from an individual having an invasive lobular carcinoma (ILC) metastasis.

Exemplary Embodiment 300: The system of embodiment 299, wherein the ILC metastasis is a bone ILC metastasis, a female reproductive system ILC metastasis, a gastrointestinal ILC metastasis, a liver ILC metastasis, or a skin ILC metastasis.

Exemplary Embodiment 301: The system of any one of embodiments 298-300, wherein the alteration comprises one or more of a substitution of one or more nucleotides, an insertion of one or more nucleotides, or a deletion of one or more nucleotides.

Exemplary Embodiment 302: The system of any one of embodiments 298-301, wherein the alteration is an alteration in PIK3CA.

Exemplary Embodiment 303: The system of embodiment 302, wherein the alteration results in one or more amino acid substitutions in a polypeptide encoded by the PIK3CA gene at amino acid position E81, R108, K111, G118, N345, D350, E365, E418, C420, E453, P539, E542, E545, Q546, E726, E970, M1004, M1043, N1044, H1047, G1049, or any combination thereof, wherein the numbering of the residues is according to the amino acid sequence of SEQ ID NO: 26.

Exemplary Embodiment 304: The system of embodiment 303, wherein the one or more amino acid substitutions comprise a E81K, R108H, K111N, G118D, N345K, D350N, E365K, E418K, C420R, E453K, E453Q, P539R, E542K, E545K, E545A, E545Q, Q546R, Q546K, E726K, E970K, M1004I, M1043I, N1044K, H1047R, H1047L, or G1049R amino acid substitution, or any combination thereof, wherein the numbering of the residues is according to the amino acid sequence of SEQ ID NO: 26.

Exemplary Embodiment 305: The system of embodiment 302, wherein the alteration results in a deletion of amino acid residue E110 (E110del) in a polypeptide encoded by the PIK3CA gene, wherein the numbering of the residues is according to the amino acid sequence of SEQ ID NO: 26.

Exemplary Embodiment 306: The system of any one of embodiments 298-301, wherein the alteration is an alteration in BRCA1 or BRCA2.

Exemplary Embodiment 307: The system of any one of embodiments 298-301, wherein the alteration is an alteration in ESR1.

Exemplary Embodiment 308: The system of embodiment 307, wherein the alteration results in one or more amino acid substitutions in a polypeptide encoded by the ESR1 gene at amino acid position E380, V418, S463, V533, L536, Y537, D538, or any combination thereof, wherein the numbering of the residues is according to the amino acid sequence of SEQ ID NO: 29.

Exemplary Embodiment 309: The system of embodiment 308, wherein the one or more amino acid substitutions comprise a E380Q, V418E, S463P, V533M, L536Q, L536H, L536P, L536R, Y537S, Y537N, Y537C, Y537D, or D538G amino acid substitution, or any combination thereof, wherein the numbering of the residues is according to the amino acid sequence of SEQ ID NO: 29.

Exemplary Embodiment 310: The system of embodiment 307, wherein the alteration results in a deletion of amino acid V422 (V422del) and/or a deletion of amino acids V533-L536 (V533_L536del) in a polypeptide encoded by the ESR1 gene, wherein the numbering of the residues is according to the amino acid sequence of SEQ ID NO: 29.

Exemplary Embodiment 311: The system of any one of embodiments 298-301, wherein the alteration is an alteration in NFL

Exemplary Embodiment 312: The system of any one of embodiments 298-301, wherein the alteration is an alteration in RB1.

Exemplary Embodiment 313: The system of any one of embodiments 298-301, wherein the alteration is an alteration in KRAS.

Exemplary Embodiment 314: The system of embodiment 313, wherein the alteration results in one or more amino acid substitutions in a polypeptide encoded by the KRAS gene at amino acid position G12, G13, L19, Q61, A146, K147, F156, or any combination thereof, wherein the numbering of the residues is according to the amino acid sequence of SEQ ID NO: 33.

Exemplary Embodiment 315: The system of embodiment 314, wherein the one or more amino acid substitutions comprise a G12V, G12D, G12A, G12R, G12S, G12C, G12L, G13D, L19F, Q61H, Q61K, Q61E, A146T, K147N, or F156L amino acid substitution, or any combination thereof, wherein the numbering of the residues is according to the amino acid sequence of SEQ ID NO: 33.

Exemplary Embodiment 316: The system of any one of embodiments 298-301, wherein the alteration is an alteration in ERBB2.

Exemplary Embodiment 317: The system of embodiment 316, wherein the alteration results in one or more amino acid substitutions in a polypeptide encoded by the ERBB2 gene at amino acid position S310, S653, V659, R678, V697, E717, T733, L755, I767, D769, G776, V777, T798, V842, L869, or any combination thereof, wherein the numbering of the residues is according to the amino acid sequence of SEQ ID NO: 34.

Exemplary Embodiment 318: The system of embodiment 317, wherein the one or more amino acid substitutions comprise a S310F, S310Y, S653C, V659D, R678Q, V697L, E717K, T733I, L755S, L755P, I767M, D769N, D769Y, D769H, G776V, V777L, T798I, V842I, or L869R amino acid substitution, or any combination thereof, wherein the numbering of the residues is according to the amino acid sequence of SEQ ID NO: 34.

Exemplary Embodiment 319: The system of embodiment 316, wherein the alteration results in a deletion of amino acids L755-T759 (L755_T759del) and/or amino acids L755-E757 (L755_E757del) in a polypeptide encoded by the ERBB2 gene, wherein the numbering of the residues is according to the amino acid sequence of SEQ ID NO: 34.

Exemplary Embodiment 320: The system of embodiment 316, wherein the alteration results in an insertion of one or more amino acid residues between amino acid residues A775 and G776, and/or between amino acid residues P780 and Y781 in a polypeptide encoded by the ERBB2 gene, wherein the numbering of the residues is according to the amino acid sequence of SEQ ID NO: 34.

Exemplary Embodiment 321: The system of embodiment 320, wherein the alteration results in an insertion of the amino acid sequence YVMA (SEQ ID NO: 51) between amino acid residues A775 and G776 (A775_G776insYVMA), and/or of the amino acid sequence GSP (SEQ ID NO: 52) between amino acid residues P780 and Y781 (P780_Y781insGSP), in a polypeptide encoded by the ERBB2 gene, wherein the numbering of the residues is according to the amino acid sequence of SEQ ID NO: 34.

Exemplary Embodiment 322: The system of any one of embodiments 298-301, wherein the alteration is an alteration in BRAF.

Exemplary Embodiment 323: The system of embodiment 322, wherein the alteration results in one or more amino acid substitutions in a polypeptide encoded by the BRAF gene at amino acid position D380, G464, G466, S467, G469, L485, L584, E586, D594, V600, or any combination thereof, wherein the numbering of the residues is according to the amino acid sequence of SEQ ID NO: 35.

Exemplary Embodiment 324: The system of embodiment 323, wherein the one or more amino acid substitutions comprise a D380H, G464R, G466E, S467L, G469A, G469E, G469R, L485F, L584F, E586K, D594N, D594G, or V600E amino acid substitution, or any combination thereof, wherein the numbering of the residues is according to the amino acid sequence of SEQ ID NO: 35.

Exemplary Embodiment 325: The system of any one of embodiments 298-301, wherein the alteration is an alteration in ARID1A.

Exemplary Embodiment 326: The system of any one of embodiments 298-301, wherein the alteration is an alteration in PTEN.

Exemplary Embodiment 327: The system of embodiment 326, wherein the alteration is a PTEN deletion.

Exemplary Embodiment 328: The system of any one of embodiments 298-301, wherein the alteration is an alteration in FGFR2.

Exemplary Embodiment 329: The system of embodiment 328, wherein the alteration results in one or more amino acid substitutions in a polypeptide encoded by the FGFR2 gene at amino acid position S252, P253, Y375, C382, M391, V395, M537, N549, K659, R664, or any combination thereof, wherein the numbering of the residues is according to the amino acid sequence of SEQ ID NO: 38.

Exemplary Embodiment 330: The system of embodiment 329, wherein the one or more amino acid substitutions comprise a S252W, P253R, Y375C, C382R, M391R, V395D, M537I, N549D, N549K, K659M, K659E, K659N, or R664W amino acid substitution, or any combination thereof, wherein the numbering of the residues is according to the amino acid sequence of SEQ ID NO: 38.

Exemplary Embodiment 331: The system of embodiment 328, wherein the alteration results in a frameshift of S799fs*22, wherein the numbering of the residues is according to the amino acid sequence of SEQ ID NO: 38.

Exemplary Embodiment 332: The system of any one of embodiments 298-301, wherein the alteration is an alteration in SMAD4.

Exemplary Embodiment 333: The system of any one of embodiments 298-301, wherein the alteration is an alteration in PTPN11.

Exemplary Embodiment 334: The system of any one of embodiments 298-301, wherein the alteration is an alteration in TERT.

Exemplary Embodiment 335: The system of embodiment 334, wherein the alteration is an alteration in the promoter of TERT, wherein the alteration in the promoter of TERT comprises a −146C>T, −139_−138CC>TT, or −124C>T mutation, or any combination thereof, wherein the numbering of the nucleotides is according to SEQ ID NO: 22.

Exemplary Embodiment 336: The system of any one of embodiments 298-301, wherein the alteration is an alteration in ALK.

Exemplary Embodiment 337: The system of any one of embodiments 298-301, wherein the alteration is an alteration in NCOR1.

Exemplary Embodiment 338: The system of any one of embodiments 298-301, wherein the alteration is an alteration in APC.

Exemplary Embodiment 339: The system of any one of embodiments 298-301, wherein the alteration is an alteration in CDH1.

Exemplary Embodiment 340: The system of embodiment 339, wherein the alteration comprises one or more deleterious CDH1 mutations.

Exemplary Embodiment 341: The system of embodiment 340, wherein the one or more deleterious CDH1 mutations result in loss of function of a CDH1 polypeptide.

Exemplary Embodiment 342: The system of embodiment 340 or embodiment 341, wherein the one or more deleterious CDH1 mutations result in a mutation of the CDH1 start codon.

Exemplary Embodiment 343: The system of embodiment 340 or embodiment 341, wherein the one or more deleterious CDH1 mutations result in one or more amino acid substitutions in a CDH1 polypeptide at amino acid position E243, D402, D433, A634, or any combination thereof, wherein the numbering of the residues is according to the amino acid sequence of SEQ ID NO: 50.

Exemplary Embodiment 344: The system of embodiment 343, wherein the one or more amino acid substitutions comprise a E243K, D402N, D433N, or A634V amino acid substitution, or any combination thereof, wherein the numbering of the residues is according to the amino acid sequence of SEQ ID NO: 50.

Exemplary Embodiment 345: The system of embodiment 340 or embodiment 341, wherein the one or more deleterious CDH1 mutations result in a stop codon at amino acid position W4 (W4*), S9 (S9*), Q16 (Q16*), W20 (W20*), Q23 (Q23*), E24 (E24*), E26 (E26*), E35 (E35*), Y37 (Y37*), E47 (E47*), R54 (R54*), E58 (E58*), R63 (R63*), Q64 (Q64*), R74 (R74*), W103 (W103*), Q129 (Q129*), R150 (R150*), Q152 (Q152*), Q177 (Q177*), Y190 (Y190*), Y228 (Y228*), E243 (E243*), Q255 (Q255*), Q264 (Q264*), Y302 (Y302*), R335 (R335*), Q346 (Q346*), E353 (E353*), Q383 (Q383*), Q449 (Q449*), E463 (E463*), Y523 (Y523*), W526 (W526*), R598 (R598*), Q610 (Q610*), Q641 (Q641*), E648 (E648*), Q699 (Q699*), Q706 (Q706*), Q765 (Q765*), Q771 (Q771*), E806 (E806*), Y827 (Y827*), or any combination thereof, wherein the numbering of the residues is according to the amino acid sequence of SEQ ID NO: 50.

Exemplary Embodiment 346: The system of embodiment 340 or embodiment 341, wherein the one or more deleterious CDH1 mutations comprise a frameshift, wherein the frameshift is an T115fs*53, P127fs*41, P200fs*6, P200fs*16, V202fs*7, H233fs*11, P277fs*5, Y302fs*1, S337fs*12, Y523fs*1, L585fs*4, 1650fs*3, 1650fs*13, A719fs*29, or Q765fs*4 frameshift, wherein the numbering of the residues is according to the amino acid sequence of SEQ ID NO: 50.

Exemplary Embodiment 347: The system of embodiment 340 or embodiment 341, wherein the one or more deleterious CDH1 mutations comprise a splice site mutation, wherein the splice site mutation is a 48+1G>A, 1565+1G>A, or 1565+1G>T splice site mutation, wherein the numbering of the nucleotides is according to SEQ ID NO: 49.

Exemplary Embodiment 348: The system of any one of embodiments 298-347, wherein the plurality of sequence reads is obtained by sequencing, whole exome sequencing, whole genome sequencing, gene-targeted sequencing, or next-generation sequencing.

Exemplary Embodiment 349: A non-transitory computer readable storage medium comprising one or more programs executable by one or more computer processors for performing a method, comprising: (a) obtaining, using the one or more processors, a plurality of sequence reads of one or more nucleic acids, wherein the one or more nucleic acids are derived from a sample obtained from an individual; (b) analyzing, using the one or more processors, the plurality of sequence reads for the presence of an alteration in one or more genes, wherein the one or more genes comprise PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, CDH1 or KMT2D; and (c) detecting, using the one or more processors and based on the analyzing, an alteration in one or more genes, wherein the one or more genes comprise PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, CDH1 or KMT2D, in the sample.

Exemplary Embodiment 350: The non-transitory computer readable storage medium of embodiment 349, wherein the sample is a sample from an individual having an invasive lobular carcinoma (ILC) metastasis.

Exemplary Embodiment 351: The non-transitory computer readable storage medium of embodiment 350, wherein the ILC metastasis is a bone ILC metastasis, a female reproductive system ILC metastasis, a gastrointestinal ILC metastasis, a liver ILC metastasis, or a skin ILC metastasis.

Exemplary Embodiment 352: The non-transitory computer readable storage medium of any one of embodiments 349-351, wherein the alteration comprises one or more of a substitution of one or more nucleotides, an insertion of one or more nucleotides, or a deletion of one or more nucleotides.

Exemplary Embodiment 353: The non-transitory computer readable storage medium of any one of embodiments 349-352, wherein the alteration is an alteration in PIK3CA.

Exemplary Embodiment 354: The non-transitory computer readable storage medium of embodiment 353, wherein the alteration results in one or more amino acid substitutions in a polypeptide encoded by the PIK3CA gene at amino acid position E81, R108, K111, G118, N345, D350, E365, E418, C420, E453, P539, E542, E545, Q546, E726, E970, M1004, M1043, N1044, H1047, G1049, or any combination thereof, wherein the numbering of the residues is according to the amino acid sequence of SEQ ID NO: 26.

Exemplary Embodiment 355: The non-transitory computer readable storage medium of embodiment 354, wherein the one or more amino acid substitutions comprise a E81K, R108H, K111N, G118D, N345K, D350N, E365K, E418K, C420R, E453K, E453Q, P539R, E542K, E545K, E545A, E545Q, Q546R, Q546K, E726K, E970K, M1004I, M1043I, N1044K, H1047R, H1047L, or G1049R amino acid substitution, or any combination thereof, wherein the numbering of the residues is according to the amino acid sequence of SEQ ID NO: 26.

Exemplary Embodiment 356: The non-transitory computer readable storage medium of embodiment 353, wherein the alteration results in a deletion of amino acid residue E110 (E110del) in a polypeptide encoded by the PIK3CA gene, wherein the numbering of the residues is according to the amino acid sequence of SEQ ID NO: 26.

Exemplary Embodiment 357: The non-transitory computer readable storage medium of any one of embodiments 349-352, wherein the alteration is an alteration in BRCA1 or BRCA2.

Exemplary Embodiment 358: The non-transitory computer readable storage medium of any one of embodiments 349-352, wherein the alteration is an alteration in ESR1.

Exemplary Embodiment 359: The non-transitory computer readable storage medium of embodiment 358, wherein the alteration results in one or more amino acid substitutions in a polypeptide encoded by the ESR1 gene at amino acid position E380, V418, S463, V533, L536, Y537, D538, or any combination thereof, wherein the numbering of the residues is according to the amino acid sequence of SEQ ID NO: 29.

Exemplary Embodiment 360: The non-transitory computer readable storage medium of embodiment 359, wherein the one or more amino acid substitutions comprise a E380Q, V418E, S463P, V533M, L536Q, L536H, L536P, L536R, Y537S, Y537N, Y537C, Y537D, or D538G amino acid substitution, or any combination thereof, wherein the numbering of the residues is according to the amino acid sequence of SEQ ID NO: 29.

Exemplary Embodiment 361: The non-transitory computer readable storage medium of embodiment 358, wherein the alteration results in a deletion of amino acid V422 (V422del) and/or a deletion of amino acids V533-L536 (V533_L536del) in a polypeptide encoded by the ESR1 gene, wherein the numbering of the residues is according to the amino acid sequence of SEQ ID NO: 29.

Exemplary Embodiment 362: The non-transitory computer readable storage medium of any one of embodiments 349-352, wherein the alteration is an alteration in NFL

Exemplary Embodiment 363: The non-transitory computer readable storage medium of any one of embodiments 349-352, wherein the alteration is an alteration in RB1.

Exemplary Embodiment 364: The non-transitory computer readable storage medium of any one of embodiments 349-352, wherein the alteration is an alteration in KRAS.

Exemplary Embodiment 365: The non-transitory computer readable storage medium of embodiment 364, wherein the alteration results in one or more amino acid substitutions in a polypeptide encoded by the KRAS gene at amino acid position G12, G13, L19, Q61, A146, K147, F156, or any combination thereof, wherein the numbering of the residues is according to the amino acid sequence of SEQ ID NO: 33.

Exemplary Embodiment 366: The non-transitory computer readable storage medium of embodiment 365, wherein the one or more amino acid substitutions comprise a G12V, G12D, G12A, G12R, G12S, G12C, G12L, G13D, L19F, Q61H, Q61K, Q61E, A146T, K147N, or F156L amino acid substitution, or any combination thereof, wherein the numbering of the residues is according to the amino acid sequence of SEQ ID NO: 33.

Exemplary Embodiment 367: The non-transitory computer readable storage medium of any one of embodiments 349-352, wherein the alteration is an alteration in ERBB2.

Exemplary Embodiment 368: The non-transitory computer readable storage medium of embodiment 367, wherein the alteration results in one or more amino acid substitutions in a polypeptide encoded by the ERBB2 gene at amino acid position S310, S653, V659, R678, V697, E717, T733, L755, I767, D769, G776, V777, T798, V842, L869, or any combination thereof, wherein the numbering of the residues is according to the amino acid sequence of SEQ ID NO: 34.

Exemplary Embodiment 369: The non-transitory computer readable storage medium of embodiment 368, wherein the one or more amino acid substitutions comprise a S310F, S310Y, S653C, V659D, R678Q, V697L, E717K, T733I, L755S, L755P, I767M, D769N, D769Y, D769H, G776V, V777L, T798I, V842I, or L869R amino acid substitution, or any combination thereof, wherein the numbering of the residues is according to the amino acid sequence of SEQ ID NO: 34.

Exemplary Embodiment 370: The non-transitory computer readable storage medium of embodiment 367, wherein the alteration results in a deletion of amino acids L755-T759 (L755_T759del) and/or amino acids L755-E757 (L755_E757del) in a polypeptide encoded by the ERBB2 gene, wherein the numbering of the residues is according to the amino acid sequence of SEQ ID NO: 34.

Exemplary Embodiment 371: The non-transitory computer readable storage medium of embodiment 367, wherein the alteration results in an insertion of one or more amino acid residues between amino acid residues A775 and G776, and/or between amino acid residues P780 and Y781 in a polypeptide encoded by the ERBB2 gene, wherein the numbering of the residues is according to the amino acid sequence of SEQ ID NO: 34.

Exemplary Embodiment 372: The non-transitory computer readable storage medium of embodiment 371, wherein the alteration results in an insertion of the amino acid sequence YVMA (SEQ ID NO: 51) between amino acid residues A775 and G776 (A775_G776insYVMA), and/or of the amino acid sequence GSP (SEQ ID NO: 52) between amino acid residues P780 and Y781 (P780_Y781insGSP), in a polypeptide encoded by the ERBB2 gene, wherein the numbering of the residues is according to the amino acid sequence of SEQ ID NO: 34.

Exemplary Embodiment 373: The non-transitory computer readable storage medium of any one of embodiments 349-352, wherein the alteration is an alteration in BRAF.

Exemplary Embodiment 374: The non-transitory computer readable storage medium of embodiment 373, wherein the alteration results in one or more amino acid substitutions in a polypeptide encoded by the BRAF gene at amino acid position D380, G464, G466, S467, G469, L485, L584, E586, D594, V600, or any combination thereof, wherein the numbering of the residues is according to the amino acid sequence of SEQ ID NO: 35.

Exemplary Embodiment 375: The non-transitory computer readable storage medium of embodiment 374, wherein the one or more amino acid substitutions comprise a D380H, G464R, G466E, S467L, G469A, G469E, G469R, L485F, L584F, E586K, D594N, D594G, or V600E amino acid substitution, or any combination thereof, wherein the numbering of the residues is according to the amino acid sequence of SEQ ID NO: 35.

Exemplary Embodiment 376: The non-transitory computer readable storage medium of any one of embodiments 349-352, wherein the alteration is an alteration in ARID1A.

Exemplary Embodiment 377: The non-transitory computer readable storage medium of any one of embodiments 349-352, wherein the alteration is an alteration in PTEN.

Exemplary Embodiment 378: The non-transitory computer readable storage medium of embodiment 377, wherein the alteration is a PTEN deletion.

Exemplary Embodiment 379: The non-transitory computer readable storage medium of any one of embodiments 349-352, wherein the alteration is an alteration in FGFR2.

Exemplary Embodiment 380: The non-transitory computer readable storage medium of embodiment 379, wherein the alteration results in one or more amino acid substitutions in a polypeptide encoded by the FGFR2 gene at amino acid position S252, P253, Y375, C382, M391, V395, M537, N549, K659, R664, or any combination thereof, wherein the numbering of the residues is according to the amino acid sequence of SEQ ID NO: 38.

Exemplary Embodiment 381: The non-transitory computer readable storage medium of embodiment 380, wherein the one or more amino acid substitutions comprise a S252W, P253R, Y375C, C382R, M391R, V395D, M537I, N549D, N549K, K659M, K659E, K659N, or R664W amino acid substitution, or any combination thereof, wherein the numbering of the residues is according to the amino acid sequence of SEQ ID NO: 38.

Exemplary Embodiment 382: The non-transitory computer readable storage medium of embodiment 379, wherein the alteration results in a frameshift of S799fs*22, wherein the numbering of the residues is according to the amino acid sequence of SEQ ID NO: 38.

Exemplary Embodiment 383: The non-transitory computer readable storage medium of any one of embodiments 349-352, wherein the alteration is an alteration in SMAD4.

Exemplary Embodiment 384: The non-transitory computer readable storage medium of any one of embodiments 349-352, wherein the alteration is an alteration in PTPN11.

Exemplary Embodiment 385: The non-transitory computer readable storage medium of any one of embodiments 349-352, wherein the alteration is an alteration in TERT.

Exemplary Embodiment 386: The non-transitory computer readable storage medium of embodiment 385, wherein the alteration is an alteration in the promoter of TERT, wherein the alteration in the promoter of TERT comprises a −146C>T, −139_−138CC>TT, or −124C>T mutation, or any combination thereof, wherein the numbering of the nucleotides is according to SEQ ID NO: 22.

Exemplary Embodiment 387: The non-transitory computer readable storage medium of any one of embodiments 349-352, wherein the alteration is an alteration in ALK.

Exemplary Embodiment 388: The non-transitory computer readable storage medium of any one of embodiments 349-352, wherein the alteration is an alteration in NCOR1.

Exemplary Embodiment 389: The non-transitory computer readable storage medium of any one of embodiments 349-352, wherein the alteration is an alteration in APC.

Exemplary Embodiment 390: The non-transitory computer readable storage medium of any one of embodiments 349-352, wherein the alteration is an alteration in CDH1.

Exemplary Embodiment 391: The non-transitory computer readable storage medium of embodiment 390, wherein the alteration comprises one or more deleterious CDH1 mutations.

Exemplary Embodiment 392: The non-transitory computer readable storage medium of embodiment 391, wherein the one or more deleterious CDH1 mutations result in loss of function of a CDH1 polypeptide.

Exemplary Embodiment 393: The non-transitory computer readable storage medium of embodiment 391 or embodiment 392, wherein the one or more deleterious CDH1 mutations result in a mutation of the CDH1 start codon.

Exemplary Embodiment 394: The non-transitory computer readable storage medium of embodiment 391 or embodiment 392, wherein the one or more deleterious CDH1 mutations result in one or more amino acid substitutions in a CDH1 polypeptide at amino acid position E243, D402, D433, A634, or any combination thereof, wherein the numbering of the residues is according to the amino acid sequence of SEQ ID NO: 50.

Exemplary Embodiment 395: The non-transitory computer readable storage medium of embodiment 394, wherein the one or more amino acid substitutions comprise a E243K, D402N, D433N, or A634V amino acid substitution, or any combination thereof, wherein the numbering of the residues is according to the amino acid sequence of SEQ ID NO: 50.

Exemplary Embodiment 396: The non-transitory computer readable storage medium of embodiment 391 or embodiment 392, wherein the one or more deleterious CDH1 mutations result in a stop codon at amino acid position W4 (W4*), S9 (S9*), Q16 (Q16*), W20 (W20*), Q23 (Q23*), E24 (E24*), E26 (E26*), E35 (E35*), Y37 (Y37*), E47 (E47*), R54 (R54*), E58 (E58*), R63 (R63*), Q64 (Q64*), R74 (R74*), W103 (W103*), Q129 (Q129*), R150 (R150*), Q152 (Q152*), Q177 (Q177*), Y190 (Y190*), Y228 (Y228*), E243 (E243*), Q255 (Q255*), Q264 (Q264*), Y302 (Y302*), R335 (R335*), Q346 (Q346*), E353 (E353*), Q383 (Q383*), Q449 (Q449*), E463 (E463*), Y523 (Y523*), W526 (W526*), R598 (R598*), Q610 (Q610*), Q641 (Q641*), E648 (E648*), Q699 (Q699*), Q706 (Q706*), Q765 (Q765*), Q771 (Q771*), E806 (E806*), Y827 (Y827*), or any combination thereof, wherein the numbering of the residues is according to the amino acid sequence of SEQ ID NO: 50.

Exemplary Embodiment 397: The non-transitory computer readable storage medium of embodiment 391 or embodiment 392, wherein the one or more deleterious CDH1 mutations comprise a frameshift, wherein the frameshift is an T115fs*53, P127fs*41, P200fs*6, P200fs*16, V202fs*7, H233fs*11, P277fs*5, Y302fs*1, S337fs*12, Y523fs*1, L585fs*4, 1650fs*3, 1650fs*13, A719fs*29, or Q765fs*4 frameshift, wherein the numbering of the residues is according to the amino acid sequence of SEQ ID NO: 50.

Exemplary Embodiment 398: The non-transitory computer readable storage medium of embodiment 391 or embodiment 392, wherein the one or more deleterious CDH1 mutations comprise a splice site mutation, wherein the splice site mutation is a 48+1G>A, 1565+1G>A, or 1565+1G>T splice site mutation, wherein the numbering of the nucleotides is according to SEQ ID NO: 49.

Exemplary Embodiment 399: The non-transitory computer readable storage medium of any one of embodiments 349-398, wherein the plurality of sequence reads is obtained by sequencing, whole exome sequencing, whole genome sequencing, gene-targeted sequencing, or next-generation sequencing.

### EXAMPLES

The present disclosure will be more fully understood by reference to the following Examples. They should not, however, be construed as limiting the scope of the present disclosure. All citations throughout the disclosure are hereby expressly incorporated by reference.

### Example 1: Profiling of Metastatic Invasive Lobular Carcinoma by Metastatic Biopsy Site

This example describes an analysis of the genomic landscape of metastatic invasive lobular carcinoma by metastatic site using a comprehensive genomic profiling workflow.

### Materials and Methods

### Comprehensive Genomic Profiling Analysis

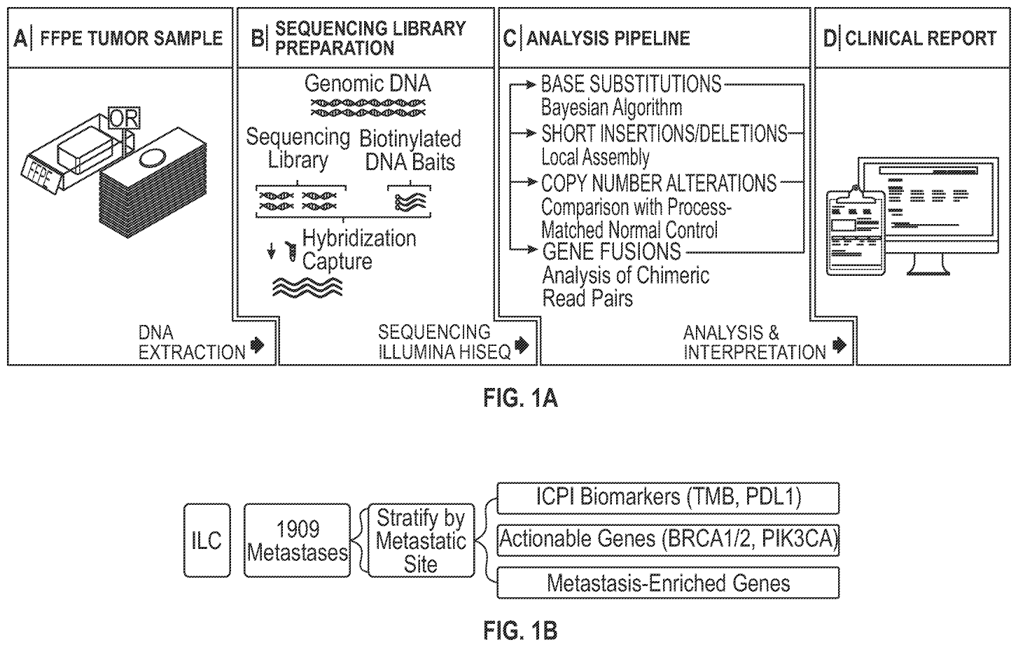

Comprehensive genomic profiling (CGP) was carried out with hybrid capture for exons from up to 395 cancer-related genes plus select introns from up to 31 genes (Frampton et al., Nat Biotechnol (2013) 31(11):1023-31). Tumor mutational burden (TMB) was determined on 0.8-1.1 Mb (Chalmers et al., Genome Med (2017) 9(1):34). VENTANA PD-L1 infiltrating cell (IC) staining (SP142; positive ≥1% staining) was available for a subset of samples. 1071 breast-biopsied and 1909 metastasis-biopsied invasive lobular carcinoma (ILC) samples, and 6926 breast-biopsied and 1901 metastasis-biopsied invasive ductal carcinoma (IDC) samples were available for analysis. FIG. 1A depicts a schematic of the CGP workflow.

### Results

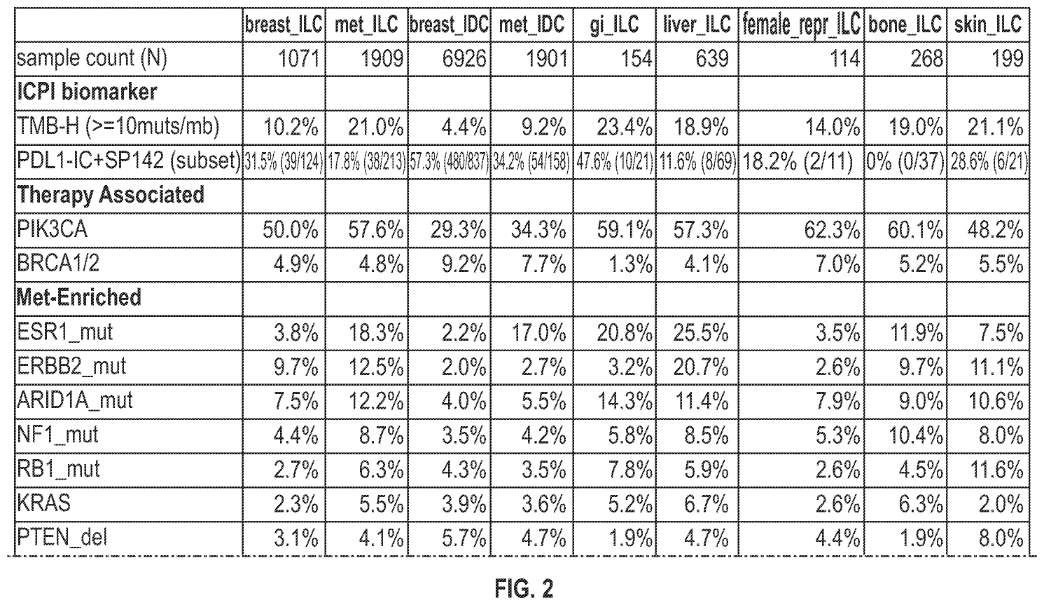

In this example, the genomic landscape of 1909 ILC metastasis (met) samples, stratified by met site, was explored Immune checkpoint inhibitor (ICPI) biomarkers, potentially actionable alterations, and therapy resistance mutations were examined. The ILC patient sample analysis workflow is depicted in FIG. 1B, while FIG. 2 summarizes the results of this analysis.

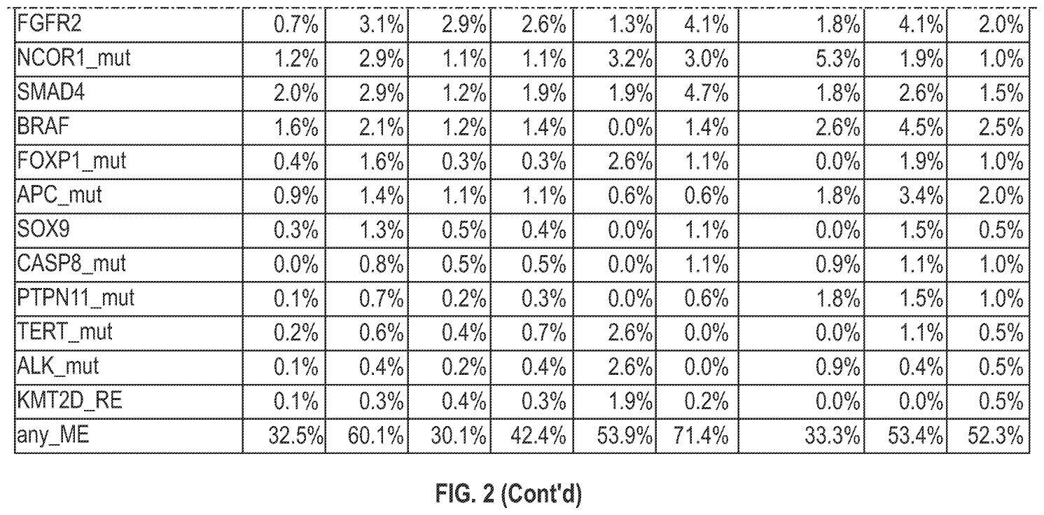

High TMB and PD-L1 IC staining may predict response to ICPIs. ILC mets overall had a greater rate of high TMB (>10 mut/Mb) relative to IDC mets (21% vs. 9%, p=7E-25) and breast-biopsied (breast) ILC (21% vs. 10%; p=9E-15) (FIG. 2). The highest frequency of high TMB (>10 mut/Mb) was observed in gastrointestinal mets (23%) and skin mets (21%) (FIG. 3). PD-L1 IC+ rates were lower in ILC mets (18%) relative to IDC mets (34%) and breast ILC (31%) (FIG. 2). However, PD-L1 IC+ rates were variable across met sites, with relatively high rates of positivity in gastrointestinal (48%), skin (29%), and female reproductive system (18%) mets, and no positive staining in bone mets (0/37) (FIG. 3).

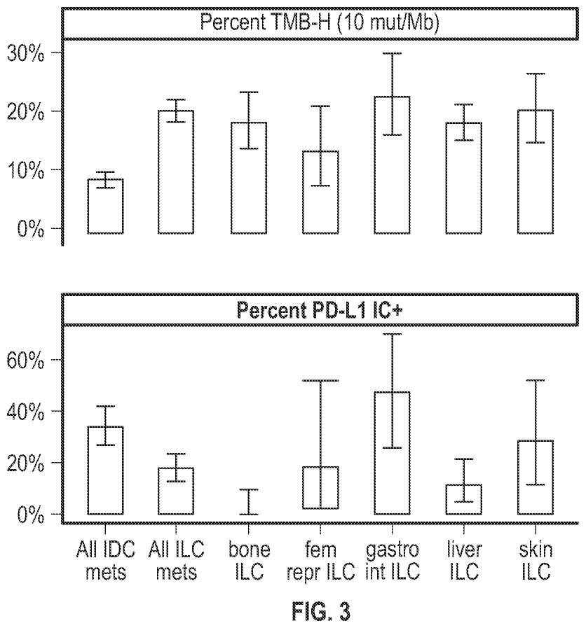

Alterations in PIK3CA, which may be targetable with kinase inhibitors, were higher in ILC mets (58%) relative to IDC mets (34%), and generally exhibited a similar frequency across ILC met sites, with modestly lower prevalence in skin mets (48%, p=0.005) (FIG. 4). Pathogenic alterations in BRCA1/2 were observed in 4.8% of ILC mets overall (FIG. 4), with a lower frequency in gastrointestinal mets (1.3%, p=0.03).

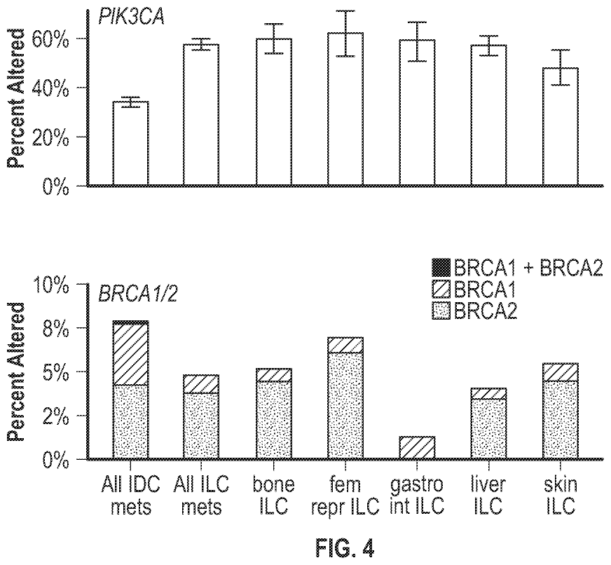

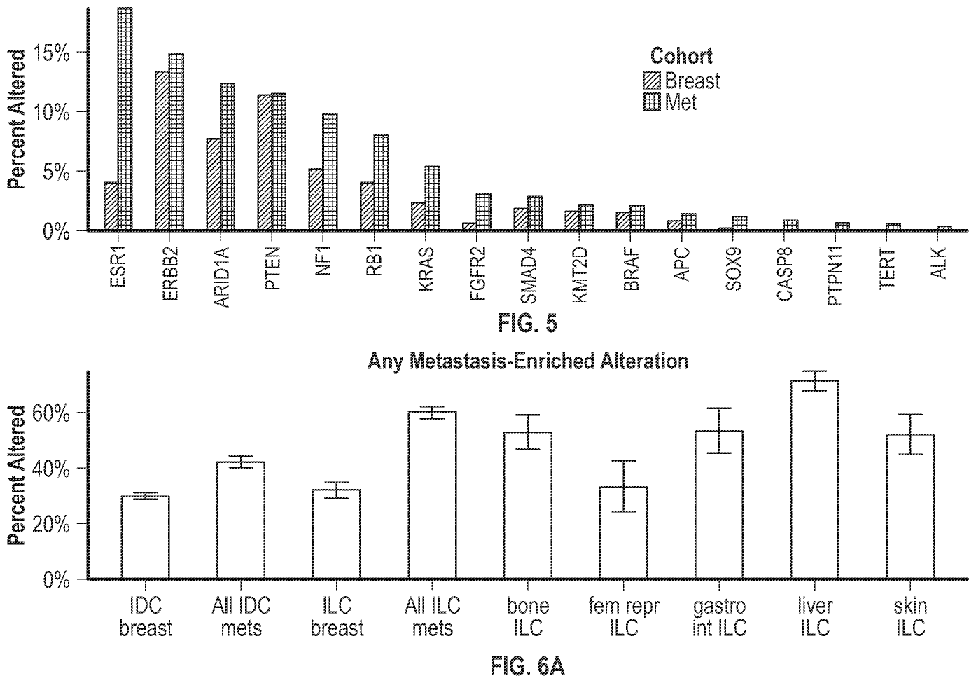

A comparison of ILC breast biopsies to ILC mets revealed 19 genes with higher prevalence in at least one ILC met site (FIG. 5). Most of these genes have known roles in therapy resistance (e.g., ESR1, NF1, RB1, KRAS, ERBB2, BRAF), though significant heterogeneity was observed across sites. As shown in FIG. 6A, met-enriched (ME) alterations were highest in ILC mets from the liver (71%) and lowest in female reproductive system mets (33%).

Potentially actionable alterations differed across met sites. For example, as shown in FIG. 6B, ERBB2 mutations, which may be targetable with HER2 kinase inhibitors, were predominantly found in liver mets (21%), with significantly lower prevalence in skin (11%), bone (10%), gastrointestinal (3%), and female reproductive system (3%) mets. ESR1 alterations were common in most ILC met sites (FIG. 6B), with the highest prevalence in liver (26%) and low frequency in female reproductive system (4%). Additionally, as shown in FIG. 2, BRAF mutations, which may be targetable by BRAF inhibitors, were observed at a higher frequency in ILC bone mets (4.5%). Moreover, while female reproductive system ILC mets harbored few ME alterations, the rare alterations were primarily found in NF1 (5%) and NCOR1 (5%) (FIG. 2).

### CONCLUSIONS

CGP revealed significant heterogeneity in ILC mets across tissues. ICPI biomarkers were variable across sites with the highest frequency in ILC gastrointestinal mets, potentially offering additional treatment avenues for these tumors. Skin mets also stood out as having an unexpectedly high frequency of ICPI biomarkers. Potentially targetable alterations in PIK3CA were common in ILC mets, with a high prevalence across sites, suggesting potential broad utility for PIK3CA inhibitors. Therapy-resistance alterations were common in ILC mets but varied across met sites. Notably, ERBB2 alterations were most prevalent in ILC liver mets, but less common at other met sites. Additionally, RB1 mutations, which are associated with CDK4/6 inhibitor resistance, were highest in gastrointestinal and skin metastases.

1. A method of treating or delaying progression of invasive lobular carcinoma (ILC) metastasis, comprising:
(a) acquiring knowledge of a tumor mutational burden (TMB) of at least about 10 mutations/megabase (mut/Mb) in a sample from an individual having an ILC metastasis; and
(b) responsive to said knowledge, administering to the individual an effective amount of an immune checkpoint inhibitor.

2-3. (canceled)

4. The method of claim 1, wherein the individual is a human.

5. The method of claim 1, wherein the ILC metastasis is a bone ILC metastasis, a female reproductive system ILC metastasis, a gastrointestinal ILC metastasis, a liver ILC metastasis, or a skin ILC metastasis.

6-10. (canceled)

11. The method of claim 1, wherein the sample from the individual comprises fluid, cells, or tissue.

12. The method of claim 11, wherein the sample from the individual comprises a tumor biopsy or a circulating tumor cell.

13. The method of claim 11, wherein the sample from the individual comprises nucleic acids.

14. The method of claim 13, wherein the sample from the individual comprises mRNA, genomic DNA, circulating tumor DNA, cell-free DNA, or cell-free RNA.

15. The method of claim 1, wherein TMB is measured in the sample by whole exome sequencing, whole genome sequencing, or gene-targeted sequencing.

16. A method of treating or delaying progression of invasive lobular carcinoma (ILC) metastasis, comprising:
(a) acquiring knowledge of a PD-L1-positive ILC metastasis in a sample from an individual having an ILC metastasis; and
(b) responsive to said knowledge, administering to the individual an effective amount of an immune checkpoint inhibitor.

17-21. (canceled)

22. The method of claim 16, wherein the ILC metastasis is a bone ILC metastasis, a female reproductive system ILC metastasis, a gastrointestinal ILC metastasis, a liver ILC metastasis, or a skin ILC metastasis.

23-29. (canceled)

30. The method of claim 1, wherein the immune checkpoint inhibitor is a small molecule inhibitor, an antibody or antibody fragment, a peptide, a fusion protein, or a nucleic acid.

31. The method of claim 1, wherein the immune checkpoint inhibitor is a PD-1 binding antagonist or a PD-L1 binding antagonist.

32. The method of claim 31, wherein the immune checkpoint inhibitor is: an anti-PD-1 antibody or antibody fragment; or an anti-PD-L1 antibody or antibody fragment.

33. The method of claim 32, wherein: (a) the anti-PD-1 antibody or antibody fragment is MDX-1106 (nivolumab), MK-3475 (pembrolizumab), MEDI-0680 (AMP-514), PDR001, REGN2810, MGA-012, JNJ-63723283, BI 754091, BGB-108, BGB-A317, JS-001, STI-A1110, INCSHR-1210, PF-06801591, TSR-042, AM0001, ENUM 244C8, or ENUM 388D4; or (b) the anti-PD-L1 antibody or antibody fragment is YW243.55.S70, MPDL3280A (atezolizumab), MDX-1105, MEDI4736 (durvalumab), MSB0010718C (avelumab), LY3300054, STI-A1014, KN035, FAZ053, or CX-072.

34-36. (canceled)

37. A method of treating or delaying progression of invasive lobular carcinoma (ILC) metastasis, comprising:
(a) acquiring knowledge of an alteration in one or more genes in a sample from an individual having an ILC metastasis, wherein the one or more genes comprise one or more of: PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, and KMT2D; and
(b) responsive to said knowledge, administering to the individual an effective amount of an anti-cancer agent.

38-39. (canceled)

40. The method of claim 37, wherein the ILC metastasis is selected from the group consisting of a bone ILC metastasis, a female reproductive system ILC metastasis, a gastrointestinal ILC metastasis, a liver ILC metastasis, and a skin ILC metastasis.

41-158. (canceled)

159. The method of claim 1, wherein the ILC metastasis comprises one or more deleterious CDH1 mutations; and/or wherein the method further comprises acquiring knowledge of one or more deleterious CDH1 mutations in a sample from the individual.

160-162. (canceled)

163. The method of claim 159, wherein the one or more deleterious CDH1 mutations result in loss of function of a CDH1 polypeptide.

164-177. (canceled)

178. A method for genomic profiling of an invasive lobular carcinoma (ILC) metastasis, comprising:
(a) detecting one or more biomarkers in a sample from an individual having an ILC metastasis, wherein the one or more biomarkers comprise one or more of:
(i) a tumor mutational burden (TMB) of at least about 10 mutations/megabase (mut/Mb),
(ii) a PD-L1-positive ILC metastasis, and
(iii) an alteration in one or more genes, wherein the one or more genes comprise one or more of: PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, and KMT2D; and
(b) providing a report to a party.

179-287. (canceled)

288. A system, comprising:
a memory configured to store one or more program instructions; and
one or more processors configured to execute the one or more program instructions, the one or more program instructions when executed by the one or more processors are configured to:
(a) obtain a plurality of sequence reads of one or more nucleic acids, wherein the one or more nucleic acids are derived from a sample obtained from an individual;
(b) analyze the plurality of sequence reads for the presence of a tumor mutational burden (TMB) of at least about 10 mutations/megabase (mut/Mb); and
(c) detect, based on the analyzing, a TMB of at least about 10 mut/Mb, in the sample.

289-292. (canceled)

293. A non-transitory computer readable storage medium comprising one or more programs executable by one or more computer processors for performing a method, comprising:
(a) obtaining, using the one or more processors, a plurality of sequence reads of one or more nucleic acids, wherein the one or more nucleic acids are derived from a sample obtained from an individual;
(b) analyzing, using the one or more processors, the plurality of sequence reads for the presence of a tumor mutational burden (TMB) of at least about 10 mutations/megabase (mut/Mb); and
(c) detecting, using the one or more processors and based on the analyzing, a TMB of at least about 10 mut/Mb, in the sample.

294-297. (canceled)

298. A system, comprising:
a memory configured to store one or more program instructions; and
one or more processors configured to execute the one or more program instructions, the one or more program instructions when executed by the one or more processors are configured to:
(a) obtain a plurality of sequence reads of one or more nucleic acids, wherein the one or more nucleic acids are derived from a sample obtained from an individual;
(b) analyze the plurality of sequence reads for the presence of an alteration in one or more genes, wherein the one or more genes comprise PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, CDH1 or KMT2D; and
(c) detect, based on the analyzing, an alteration in one or more genes, wherein the one or more genes comprise PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, CDH1 or KMT2D, in the sample.

299-348. (canceled)

349. A non-transitory computer readable storage medium comprising one or more programs executable by one or more computer processors for performing a method, comprising:
(a) obtaining, using the one or more processors, a plurality of sequence reads of one or more nucleic acids, wherein the one or more nucleic acids are derived from a sample obtained from an individual;
(b) analyzing, using the one or more processors, the plurality of sequence reads for the presence of an alteration in one or more genes, wherein the one or more genes comprise PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, CDH1 or KMT2D; and
(c) detecting, using the one or more processors and based on the analyzing, an alteration in one or more genes, wherein the one or more genes comprise PIK3CA, BRCA1, BRCA2, ESR1, ERBB2, ARID1A, NF1, RB1, KRAS, PTEN, FGFR2, NCOR1, SMAD4, BRAF, FOXP1, APC, SOX9, CASP8, PTPN11, TERT, ALK, CDH1 or KMT2D, in the sample.

350-399. (canceled)


SEQUENCE LISTING (52 sequences)

SEQ ID NO: 1 - DNA, 9415 bp, Homo sapiens
atgttgtttttcctgtacctaagttcccgctgtaggtggtacctttgcagacggtgcgatgggggaagcagagaagtttcactacatctatagttgtgacctggatatcaacgtccagcttaagataggaagcttggaagggaagagagaacaaaagagttataaagctgtcctggaagacccaatgttgaagttctcaggactatatcaagagacatgctctgatctttatgttacttgtcaagtttttgcagaagggaagcctttggccttgccagtgagaacatcctacaaagcatttagtacaagatggaactggaatgaatggctgaaactaccagtaaaataccctgacctgcccaggaatgcccaagtggccctcaccatatgggatgtgtatggtcccggaaaagcagtgcctgtaggaggaacaacggtttcgctctttggaaaatacggcatgtttcgccaagggatgcatgacttgaaagtctggcctaatgtagaagcagatggatcagaacccacaaaaactcctggcagaacaagtagcactctctcagaagatcagatgagccgtcttgccaagctcaccaaagctcatcgacaaggacacatggtgaaagtagattggctggatagattgacatttagagaaatagaaatgataaatgagagtgaaaaacgaagttctaatttcatgtacctgatggttgaatttcgatgtgtcaagtgtgatgataaggaatatggtattgtttattatgaaaaggacggtgatgaatcatctccaattttaacaagttttgaattagtgaaagttcctgacccccagatgtctatggagaatttagttgagagcaaacaccacaagcttgcccggagtttaagaagtggaccttctgaccacgatctgaaacccaatgctgccacgagagatcagttaaatattattgtgagttatccaccaaccaagcaacttacatatgaagaacaagatcttgtttggaagtttagatattatcttacgaatcaagaaaaagccttgacaaaattcttgaaatgtgttaattgggatctacctcaagaggccaaacaggccttggaacttctgggaaaatggaagccgatggatgtagaggactccttggagctgttatcctctcattacaccaacccaactgtgaggcgttatgctgttgcccggttgcgacaggccgatgatgaggatttgttgatgtacctattacaattggtccaggctctcaaatatgaaaattttgatgatataaagaatggattggaacctaccaagaaggatagtcagagttcagtgtcagaaaatgtgtcaaattctggaataaattctgcagaaatagatagctcccaaattataaccagcccccttccttcagtctcttcacctcctcctgcatcaaaaacaaaagaagttccagatggcgaaaatctggaacaagatctctgtaccttcttgatatcgagagcctgcaaaaactcaacactggctaattatttatactggtatgtgatagtggaatgtgaagatcaagatactcagcagagagatccaaagacccatgagatgtacttgaacgtaatgagaagattcagccaagcattgttgaagggtgataagtctgtcagagttatgcgttctttgctggctgcacaacagacatttgtagatcggttggtgcatctaatgaaggcagtacaacgcgaaagtggaaatcgtaagaaaaagaatgagagactacaggcattgcttggagataatgaaaagatgaatttgtcagatgtggaacttatcccgttgcctttagaaccccaagtgaaaattagaggaataattccggaaacagctacactgtttaaaagtgcccttatgcctgcacagttgttttttaagacggaagatggaggcaaatatccagttatatttaagcatggagatgatttacgtcaagatcaacttattcttcaaatcatttcactcatggacaagctgttacggaaagaaaatctggacttgaaattgacaccttataaggtgttagccaccagtacaaaacatggcttcatgcagtttatccagtcagttcctgtggctgaagttcttgatacagagggaagcattcagaacttttttagaaaatatgcaccaagtgagaatgggccaaatgggattagtgctgaggtcatggacacttacgttaaaagctgtgctggatattgcgtgatcacctatatacttggagttggagacaggcacctggataaccttttgctaacaaaaacaggcaaactcttccacatagactttggatatattttgggtcgggatccaaagcctcttcctccaccaatgaagctgaataaagaaatggtagaaggaatggggggcacacagagtgagcagtaccaagagttccgtaaacagtgttacacggctttcctccacctgcgaaggtattctaatctgattttgaacttgttttccttgatggttgatgcaaacattccagatattgcacttgaaccagataaaactgtgaaaaaggttcaggataaattccgcttagacctgtcggatgaagaggctgtgcattacatgcagagtctgattgatgagagtgtccatgctctttttgctgcagtggtggaacagattcacaagtttgcccagtactggagaaaatgaaactgggattgacccatcaagatgcttggctcaataagaaaaccacgttaggagcaacctttgtatattggagacttcagagtaaccagcaaggaagagaaatcttaatcttcaagttaccatattttccaaatattacatggtacctgagttctgcttccttggatgtcattgcttaaatatagtcttgaagggcttgttttgaaatattgtatatattttttcaaatgtatacattgttaataaattaagaaatgagaaacattcttatttatgtacatgttatgagagttctggaggaagtaatatgttgaaatagtaaaacattttagtttttgagtttaaaatgttattttttcctcttatatcttcatatcaggacagcagtaacctgaatttagattgtcacagtttacaagtttttgtttgttccttctttcctctctcttctcccactcacttttgtgtgttgtttgtttttgcctaatttacatgtttaaagttctacttaaaatttttcttcaagttttacatgtgatggctcattcttctgcagttcaagaattgcttatatttttagaagatactcattttctcttacaaataattaattttttgtaaactacagttaatagttcttctagcttgtaagtgtgtaaagtagaatatgaaaatgtggctatttagataactggccaaaaggtcatatgaccaccagttttagtattgagcataagatatttttacatattcatgtaagacaatgtaaattgtctttctgcaataaattttttggatgcaaatttagatttcaaaagctttttgatgttataaaggagttgaggaacaaaaagctctttgaaatagctcacctagacctgttaatgtaatatattctttctgaattaagcaaaattttttttgtcttttaagactagaactaatccctctagcttttttactggaactttggggaaaaagaacaaatacaaatatacaaatgatatgaacaatatggtaataatcttttgcaacttaatatgttacatagtcatatgacttggtcttggaagaaaaagaattgttttggtgctgaataaatattagaaatcaagtgacagagaagatgaaggtaaaattgtggcacagtttcagagaatgccgttaatgataacgtgttatttgtctcaaattcttagccctgggcttgcctcttcagagtggggttaccggcgagagggcaagcatgtaattaagagacactctagaggcatatggaatatagtttgtataaaacatacctgctgattagaaaaatggtaaaaatgaaaatacctttgtacatcatcttaagagagcttatatataaactaaatgggcaggctccatcgtctacctttctttttcttttcttcacatgtttatgtcttttttgttattagtcagacatttttagccctctgtatgctcattcttgtagttctatttctattgaaacttcctccaaaacttttctctgaattacccttgttgaaactgacctttcttccgtgtgaactcctacacttgcatctcctatttcaggatttggcatactttttctttaatgggctaagaaagtaaatatcttcagctttggggggcatatgatcccaatagcatcaacttaactctgctaactggaaggcaaaagtgtatcatcttactcgtctttataaactccacagcactctgcatagatatgaccatgtttataaggaactcaataaataattttgacacaaacatgaaattctagaagggcagacactgacaactagtaaggaagttgatcatgagtaactttgtattcagtgccaccttaacaagaattgtaaagaggataaacctttctaggaaggttgatgcgtgttgggtgaggaaggagcatctctcaggagggtaaacagtagctgtggttttgctgagagattttggctgtgagtgacaagcctatatcaaaaagaaaagtggcttatcaatctcagtttttattgctcaaagcaattctttactcctattttttccaaattccattgtttttactctttagagctctatcctcagagtgtagtctgcatatttctttattatcattgccatgtgacttctttggtgacaagttcagtgatatttcaagcatgatgttttgagtttgagaaatgtatttgtgaggacacatcactttaattaaaaaagtgtgagtactatggtcattaagttattcagagtgagatttctgagaggagaagagagaatgtagatggcagttattgagagtggggctataggacaaaaagaagaaaagtatggttaacattgccaaataaagggtcatacagccttgctttttgctggagttagcaaaaatcattctccatatgatagttttagatgttccccagctcatttgtattattgagttttcagctgggtatatattataataacagtcaacatgatgtaggggctcagtattacatggcagggctatactaattgcttcatatgccttatctcattattctgtgagatagaattgtctcctcattttttagttgaaataactgaagtttaaagcaattgaatcatctgcccaaggataagctgctggtgagagcagagttgggatttgaagtcgagttagaccccagtgatcacagtcttgacgattaaattcttccagctttcatttttcactgagataatggtagtgatagtactgacctctaatgtgtgcatttgtgggtatgtggtccattcagctttaatccccagaagacaaggcttattccttttcttatttttggtcatgttttatttttccattgcttttaacaggattaccaaaggcacactcagtagtcagtaaacacatttctaggaaaggtgttgtgtcatcatgccacatattcatactttccttgggttggaaaatagatcattcagtaaaaacatacaggaaaaatgaatcttgccaatgcaattgttaacctacaaccataatataccttaagtatatttttgcacataagtataacattgcgatttaaaacaataaaccagattgagattctaaggagcattttgtaagtaattactaatgtttattttagagagatcacacaacttcaaataaaaactgacatagattgaacaccttgagaataaactttagtgccaaatggaaaataattttttacaagtaaatttgaagaacaatgtgaactttctataattatatacagaaaatatactgatttgccaaaatgagtaattttgatatattaatatttcacttataagaatgcataccacctgatccaggatgggatccaggaacagaaaaagaacattaggtaaaaatgacagaaatctgaatatagtatagagtagctaaaaacaaaccaaaaaaaaaaaaaaaaaaaaaggaaaaacagaataagcctcagaagatagtgagcatcccacctgggtgaccacttagagaagctgaggtgggacccctgcatccgataagtggctgcactagaccacaggaaccagactccactgtaagaactagagtgggaatggacatgtcaaaacctcctgggaaccttattaaatgtgcacattcccaatccccactcctgagcagccaaatcaatctgcaggtgggccttgtgagttttcccaggggattccgatgcctgctagatttaaccagtagatgagatgttaagttatcctgtagtccagagattccacaggccagtgtcagggaacaggggccagggctgggaaggcataagaacagaagataaatttgaggctctgttacgctggtcatccaacccaaccaggtatctcttgttatatgctaacttcattttccctttcttgtccatagtgggaaaaaataatttttaactggctatgtagttaaaaaacctaacaaaaagcacttttctatataatgagtggtttatattaagaataggtacaatgtaaatactggtaagtgaaagttcattttgttcagtgctatgggctggtacattgcatttcaacactagttttttacttttatctaccctgagggtagataacccatattccaaaatacagtattattttcatttgctgtaacattttaagaaatatgtagtttctaaaacttcatatactttatatggaatatgaacaactttaatatgaataaatagtctagaaaaaaatttaggaaaaattgccatgaaatcatgagctaagtcaatggttattttaaaccacttaattttgagactagtatttcatacagaaaaagctaatactgatatcacacactaattgtgtataaaacactaggaatgtcaaaatagaacataagaaaatgagtctagtggacccactgcatagtgtaactttgggatgaatttaaaagaatatttaagtcacagtcaattttctgttggtctaattttaccaaataaccccctaaataagaagtctgatttatctaaaatagaagttaacatagcatcccagtattgccattctaaaactctggcatgtaaatgggagaaatggaaataagctcaaaatgggtagagtaacccatttctgcatgaaccatagaatttaactagaatttggctggctcatgcctgtaatgccagcactttgggaggccaaggcaggcagatcacttgagcccaggagtttgagaccagcccgggcaacatggtgaaactccatctcttcaaaaatatatatttgtgtgtgtgtgtgtgtgtatgtgcgtgcacaaaaattagctgagtgtggtggttcacacttgtaatcacagctacctgggaggctgaggcaggaggattgcttgagcctgggaggcagaggttgcagtgagctgagattgcaccattgcactgcagcctgggtaatagagcgagacaactctatctcaaaaaaaagaatttcactagaatttatatcttcagtgtttgcattagattgtcaccagcactatctgccttgtgactgtcaggactaatccttatatcatttatcttatgtcctttacctcagtttatgtctgtcaccacaagaaacccagacctccctaatcataccatatgccctggtcttacccacatacagctcggtacaactgcatttccccaaacctcagaactccttgggccatcactgtcagagacatttgaactagagcaactccatcttgagtagggactgggtataatgaagctgagacccactgggctgcattcccaggaagttaggcattcttagtcacaggatgagataggagattggctcaagataaggtcacaaagaccttgctgataaaacagtctgtggtaaagaagccagccaaaacccaccaaaaccaagacgggaatgaaagtgacctccggtcatcctcactgctcattatacgctagttataatgcattgacatgctaagagacactcccaccagcaccatgacagtttacaaatgccataacaacatcaggaagttaccctatatggtctaaaaaggggaggaaccctcagttctgagaatttccttgaaaattcatgaataatccaccccttgtttagcatatgatcaagaaataatcattaaaaaatagccaaccagcagctcatgctgctggtctgcctatggagtagccatcctttctttattaacctgctttcactttatggactcccctgaattcttctgcaggagatccaagaaccctctcttggagtcttaaccaggatccctttccagtaacttcagcaggatctgtgagtgccagtttccttctgtggtgcaaaactaggtgctaagtacactttttcccccctttcctctatttttaaaagaactttcagcccttttgaagtgggtacattgtctggagtacataccctggggttcattgttataggagaatttaggacacagacacacgaggagtttaggagcagaggtttaataggtagaagagaagggaaagagaaacagcttcctctatagatgtctgcactgacaccgtggctttaaggcatggcccatgatgtgtctccgagtggaaaagactggttggtggtgaatgcgccaaattttatagtccggtttgaggaggtggtttctgattggttgcatcatgtatgacgtttgcaaggtgcatgtggaaggctggtcgccccaccctaatcttatgcaaatgggctttccagttgattgggtccatcttgtgtaacacgtggctggcagagaagggaagatgaagccaccatcttgaaaatgtctagtccttagtttctgctggcattcacccatgcaagctgccagcttgtctatgtctgcagctcaactttacaggctgctctttgttagcaaatgatttggagcttcttttcattaaagaaaaaagccttaccgaggactcccatacccttgctttctgcttaagtgatttcttcttaactcctatgtcaattttatacagcatgaaataatgatgtatctataaatgagcctttgcaagattaccatttatcaaagaaaaccagtgatgtttagttaactgtttgtaaaaataaatggttagtagaatgtctaattcaataaatgtttcataaatgagtaaa

SEQ ID NO: 2 - DNA, 9259 bp, Homo sapiens
agttccggtgccgccgctgcggccgctgaggtgtcgggctgctgctgccgcggccgctgggactggggctggggccgccggcgaggcagggctcgggcccggccgggcagctccggagcggcgggggagaggggccgggaggcgggggccgtgccgcccgctctcctctccctcggcgccgccgccgccgcccgcggggctgggacccgatgcggttagagccgcggagcctggaagagccccgagcgtttctgctttgggacaaccatacatctaattccttaaagtagttttatatgtaaaacttgcaaagaatcagaacaatgcctccacgaccatcatcaggtgaactgtggggcatccacttgatgcccccaagaatcctagtagaatgtttactaccaaatggaatgatagtgactttagaatgcctccgtgaggctacattaataaccataaagcatgaactatttaaagaagcaagaaaataccccctccatcaacttcttcaagatgaatcttcttacattttcgtaagtgttactcaagaagcagaaagggaagaattttttgatgaaacaagacgactttgtgaccttcggctttttcaaccctttttaaaagtaattgaaccagtaggcaaccgtgaagaaaagatcctcaatcgagaaattggttttgctatcggcatgccagtgtgtgaatttgatatggttaaagatccagaagtacaggacttccgaagaaatattctgaacgtttgtaaagaagctgtggatcttagggacctcaattcacctcatagtagagcaatgtatgtctatcctccaaatgtagaatcttcaccagaattgccaaagcacatatataataaattagataaagggcaaataatagtggtgatctgggtaatagtttctccaaataatgacaagcagaagtatactctgaaaatcaaccatgactgtgtaccagaacaagtaattgctgaagcaatcaggaaaaaaactcgaagtatgttgctatcctctgaacaactaaaactctgtgttttagaatatcagggcaagtatattttaaaagtgtgtggatgtgatgaatacttcctagaaaaatatcctctgagtcagtataagtatataagaagctgtataatgcttgggaggatgcccaatttgatgttgatggctaaagaaagcctttattctcaactgccaatggactgttttacaatgccatcttattccagacgcatttccacagctacaccatatatgaatggagaaacatctacaaaatccctttgggttataaatagtgcactcagaataaaaattctttgtgcaacctacgtgaatgtaaatattcgagacattgataagatctatgttcgaacaggtatctaccatggaggagaacccttatgtgacaatgtgaacactcaaagagtaccttgttccaatcccaggtggaatgaatggctgaattatgatatatacattcctgatcttcctcgtgctgctcgactttgcctttccatttgctctgttaaaggccgaaagggtgctaaagaggaacactgtccattggcatggggaaatataaacttgtttgattacacagacactctagtatctggaaaaatggctttgaatctttggccagtacctcatggattagaagatttgctgaaccctattggtgttactggatcaaatccaaataaagaaactccatgcttagagttggagtttgactggttcagcagtgtggtaaagttcccagatatgtcagtgattgaagagcatgccaattggtctgtatcccgagaagcaggatttagctattcccacgcaggactgagtaacagactagctagagacaatgaattaagggaaaatgacaaagaacagctcaaagcaatttctacacgagatcctctctctgaaatcactgagcaggagaaagattttctatggagtcacagacactattgtgtaactatccccgaaattctacccaaattgcttctgtctgttaaatggaattctagagatgaagtagcccagatgtattgcttggtaaaagattggcctccaatcaaacctgaacaggctatggaacttctggactgtaattacccagatcctatggttcgaggttttgctgttcggtgcttggaaaaatatttaacagatgacaaactttctcagtatttaattcagctagtacaggtcctaaaatatgaacaatatttggataacttgcttgtgagatttttactgaagaaagcattgactaatcaaaggattgggcactttttcttttggcatttaaaatctgagatgcacaataaaacagttagccagaggtttggcctgcttttggagtcctattgtcgtgcatgtgggatgtatttgaagcacctgaataggcaagtcgaggcaatggaaaagctcattaacttaactgacattctcaaacaggagaagaaggatgaaacacaaaaggtacagatgaagtttttagttgagcaaatgaggcgaccagatttcatggatgctctacagggctttctgtctcctctaaaccctgctcatcaactaggaaacctcaggcttgaagagtgtcgaattatgtcctctgcaaaaaggccactgtggttgaattgggagaacccagacatcatgtcagagttactgtttcagaacaatgagatcatctttaaaaatggggatgatttacggcaagatatgctaacacttcaaattattcgtattatggaaaatatctggcaaaatcaaggtcttgatcttcgaatgttaccttatggttgtctgtcaatcggtgactgtgtgggacttattgaggtggtgcgaaattctcacactattatgcaaattcagtgcaaaggcggcttgaaaggtgcactgcagttcaacagccacacactacatcagtggctcaaagacaagaacaaaggagaaatatatgatgcagccattgacctgtttacacgttcatgtgctggatactgtgtagctaccttcattttgggaattggagatcgtcacaatagtaacatcatggtgaaagacgatggacaactgtttcatatagattttggacactttttggatcacaagaagaaaaaatttggttataaacgagaacgtgtgccatttgttttgacacaggatttcttaatagtgattagtaaaggagcccaagaatgcacaaagacaagagaatttgagaggtttcaggagatgtgttacaaggcttatctagctattcgacagcatgccaatctcttcataaatcttttctcaatgatgcttggctctggaatgccagaactacaatcttttgatgacattgcatacattcgaaagaccctagccttagataaaactgagcaagaggctttggagtatttcatgaaacaaatgaatgatgcacatcatggtggctggacaacaaaaatggattggatcttccacacaattaaacagcatgcattgaactgaaaagataactgagaaaatgaaagctcactctggattccacactgcactgttaataactctcagcaggcaaagaccgattgcataggaattgcacaatccatgaacagcattagaatttacagcaagaacagaaataaaatactatataatttaaataatgtaaacgcaaacagggtttgatagcacttaaactagttcatttcaaaattaagctttagaataatgcgcaatttcatgttatgccttaagtccaaaaaggtaaactttgaagattgtttgtatctttttttaaaaaacaaaacaaaacaaaaatccccaaaatatatagaaatgatggagaaggaaaaagtgatggttttttttgtcttgcaaatgttctatgttttgaaatgtggacacaacaaaggctgttattgcattaggtgtaagtaaactggagtttatgttaaattacattgattggaaaagaatgaaaatttcttatttttccattgctgttcaatttatagtttgaagtgggtttttgactgcttgtttaatgaagaaaaatgcttggggtggaagggactcttgagatttcaccagagactttttctttttaataaatcaaaccttttgatgatttgaggttttatctgcagttttggaagcagtcacaaatgagacctgttataaggtggtatttttttttttcttctggacagtatttaaaggatcttattcttatttcccagggaaattctgggctcccacaaagtaaaaaaaaaaaaaaatcatagaaaaagaatgagcaggaatagttcttattccagaattgtacagtattcaccttaagttgattttttttctccttctgcaattgaactgaatacatttttcatgcatgttttccagaaaatagaagtattaatgttattaaaaagattattttttttattaaaggctatttatattatagaaactatcattaatatatattctttatttacatgatctgtcccatagtcatgcattgttttgcaccccaaattttttattgttcatagcagcatggtcagctttcttcttgatctatagatgaggctcaggcactatcccatttataccaataaccagtgtataactacttaaggaaaacataaaaacttcatcttctttccttttatttcttatgtgaatctcccgtcttccattctcttttataattgagaatgtctcaatcatatgaaattagttaccagaattaacacaatttagactatcttcctgattccttaaacccctttactgaagtatactcatgaataatactttaaaatatgggggaatagaaaccatgaactttttacctttttaaactatttatccatatctccaaagtagaacattaaaccattttaagatatgtctcattcccaagtagtcagagctcactctccaactttattaaatactatttgagcacaggacacattcttaaacattttgaaaaacattaacccaagatgtagaggctactgctagtcgtcattctagaatctgatattttactctgtatttgaaatgaatgattaatgtcctaggaaattagctttagcagatgtccaggtgccacatcaaaaaagtgcaataattattgacagttttttagattaggcatattattggaaaacaactttataaagagtgaacattgtatactctagtaaaacagcatcactttaaaaatattcatttatgaaatctgttacctatagttgaagtcttgagtagtgaacaagggactctaataccaatactcttaatatctggctattttagatcccttaaagggcataattattggaaatttaggtatttcactaaagcatgtatataatattgccaacaagaaaagtaaatttgaagattaagggaacttacttctgcaaactgtcttgcgatagttaagcagaatttaaactctgttttaagcaggaaaccagaaagattattttgcagttgtagaagatttcataacttattaaaacttattaacattttgtgttgtttagatataggcagttgatacatactaacatcccagccttttcaatatcagggttaaattataggaaaactcagtaaaatggtacaaatctgaaagtttgatggtagaaactgaagatttaacagagaactgtgttttacccgagtgccaaaaatgctgtgagcctccttgcacaaaatttataccacttttgcatttttatctatcagtccagatagttgtctcccctccttctcccaggacctctccaccattaaaatgcacaaaccacatggccgatttcaccatttacatttattttcaaaagttactacaaccaaattaattctattagaagaaatgtagacaaattctataaagactatagattgtgacctaagaaagaaatgaggcaaagaaccaaacattgaattaaatgctacatgggtgactaagatctgtttcaagtcagtgataatatagccacttctgggtacttcagtatcagagatcagttctcgtggtttagacagttcctatctatagctgactatccttgtccttgaatatggtgtaactgactattggctctacagttttattgggccacttaagaaatatttccttgaataattattttgagaaaaagtctaaaagtaataaaaataattttaaacacactgtagtaagaaatgactgttggaaaattatgctttcactttctaccatattctcagctatacaaaaccatttattttgaagatttttagactactgttaatttgaaatctgttactcttattgtggaatttgtttttttaaaaaagatgtttctaattggatttttaaaagaagaatggaatttggttgctattttacaatagaacctaagctttttgtggttcttagtgtcctatgtaaaacttagtgtcaaagtaatcaactttgagattttcccttctattctgctttatattaaaagcccattagaaaatgggaacctggtgaatatataatgaattgtaaaatattttaatgtgtaactttttcaactgtgaaactgacttgattttttgatgaaaacagctgctgataaagtattttgtgtaaagtgtagttcttattaatcaggaaaatgatgacttgattagactgtatatgccctcttggattttattttaaatggattggtgactttcacataggtaaaacacagtccatctgtattcttttttccatcaaaaatcgagtgatttggaattataaaaaaattgtgagcagcctatttgaaaggcatcatggaaatttcacagcacaataacacggatttgttttttcttaatgatgtaaatccgtttaattcatactttgatcaatagcccatgcttgccaactctgaagaaatttaatttccagcagtattttaaagctagcctgttaactttttctgaatatttaaagttcctcttttttctatgtctgcacaaactgcagacctgggctggacccacatactcaagagtccaccttaagaaattattttgatgtccaagacatcactaaaatatttaagtttaaagataatatgtggtgttaatagattgtggtgcttttactatttaaagacaactttcatacttcagatgtttttgagaagaggggaatgtgaggggagggggcagaacagggaggagttgtttgaatgaattacattctttatatccatcctgctcatttggggcatgtctttaagagaaggctgaaagttgtgagagtatattgtataccgtaagagaatcaactcttcatcatggatgggattgtgaaggctgaactataaaattcagcattgacagcatcctcaattaataattcttggtgacagaataatacagctgggctgttttttaaaatataaacaataccatttttaattattacattaaaaattgtaaatatatctatgtgccatggcctgggaagcctgctttcttttttcataaaaattatttttactgtatgaaaagatcatggggtttagctcaaaatatctgtggtcctgataaaattggattggtaactctacctcagaaggaaaatgggaaaaaaaaatagatgagtcacaattcaatacttcaagctcagaaactgtgcagatcactgaattttagatttataaagtcagagttggcatgccttgtttttaatgatatggaagaccttaagaaaaaaacttggctgaagtttaatcgttggtccagccatttgaaaaaggcaatagtttgaggaggttcccgaattcggcatttgaaattcattttgttctctcttcttcattattagtgcatttggtgtgtgtatacttgcacacaattctgtttgtgtacacactgcttgcttagccctagtcaagaggcatcttttataaaaggtgtaaagaaatatcaaggttctaaaattcggaagagtttagaatttattaggagtttcccaagttgggatgttagtctttaaataaacttcatgcacctattccacttaaggttttgcacctcctttttattagtgcagtgccatttcttctgcttgattttaggtatgttaatattccagccttgctagttagcataaagtgacaggtgtgagccatgaggaaattttctgacttaatttgtacacaactacatataagagttttagtggaggaaaaaaattagtcccttgtgcgtatacagtagttaggtaaatgatttttctaccaacagtatactccattcctcatgtaggtaagtacagaaaaggtttttaaatgtatttttttagccagttaaagtctatgaatctatctgcaaccttatttaatctgtcactataataattttgtggttatgctaagaaccatgtatacttttaggtattcttatttttgtcaatttttctaggttggcaaggaggcagaaaaccttcattgtttcatattaaaatataattagactaaacttaattctagtatgaatttccaaaatcattatctatttatttcatttttatttaattttgtttttatttcatttttaaaagtcccttgttcaatttaacttatgttcctaagagaggttggagaacttggccttcatctgatttcaaaaatgttttgagtttcaaatgaagttaatggtttcagtgtgattcagtcctcagacctaattgggttgaataaaatctaaaagaatatacccttttggagcataacattttaataccttggggaatgtggcactaccaaaagaagactactaacacgtcagatgttcacctggaagctttatcaagaaattcgaaccacccttttggccccattaattgtagcaagtttatttctctatattttgtcattcagtgaattgaagtcctgtggtatactgcattcattagaagaaaaacgtttttaatgtccttttaatgatggcccagaaagcatttgacacagcaagatgcatgtgttactatattgagaatatagaataataacagtatcactaaatttaagacctcttcccagtcttgctgttcctagcaagaagtttggcctgtgactgcacttactgtttatgctcatcagaaactgtcaatgtctgcttttctttaactctgcagtctgtaacatcacgctgtttattaaaaaaaaaaagaaaaatta

SEQ ID NO: 3 - DNA, 7088 bp, Homo sapiens
gctgagacttcctggacgggggacaggctgtggggtttctcagataactgggcccctgcgctcaggaggccttcaccctctgctctgggtaaagttcattggaacagaaagaaatggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtagttctgtttcaaacttgcatgtggagccatgtggcacaaatactcatgccagctcattacagcatgagaacagcagtttattactcactaaagacagaatgaatgtagaaaaggctgaattctgtaataaaagcaaacagcctggcttagcaaggagccaacataacagatgggctggaagtaaggaaacatgtaatgataggcggactcccagcacagaaaaaaaggtagatctgaatgctgatcccctgtgtgagagaaaagaatggaataagcagaaactgccatgctcagagaatcctagagatactgaagatgttccttggataacactaaatagcagcattcagaaagttaatgagtggttttccagaagtgatgaactgttaggttctgatgactcacatgatggggagtctgaatcaaatgccaaagtagctgatgtattggacgttctaaatgaggtagatgaatattctggttcttcagagaaaatagacttactggccagtgatcctcatgaggctttaatatgtaaaagtgaaagagttcactccaaatcagtagagagtaatattgaagacaaaatatttgggaaaacctatcggaagaaggcaagcctccccaacttaagccatgtaactgaaaatctaattataggagcatttgttactgagccacagataatacaagagcgtcccctcacaaataaattaaagcgtaaaaggagacctacatcaggccttcatcctgaggattttatcaagaaagcagatttggcagttcaaaagactcctgaaatgataaatcagggaactaaccaaacggagcagaatggtcaagtgatgaatattactaatagtggtcatgagaataaaacaaaaggtgattctattcagaatgagaaaaatcctaacccaatagaatcactcgaaaaagaatctgctttcaaaacgaaagctgaacctataagcagcagtataagcaatatggaactcgaattaaatatccacaattcaaaagcacctaaaaagaataggctgaggaggaagtcttctaccaggcatattcatgcgcttgaactagtagtcagtagaaatctaagcccacctaattgtactgaattgcaaattgatagttgttctagcagtgaagagataaagaaaaaaaagtacaaccaaatgccagtcaggcacagcagaaacctacaactcatggaaggtaaagaacctgcaactggagccaagaagagtaacaagccaaatgaacagacaagtaaaagacatgacagcgatactttcccagagctgaagttaacaaatgcacctggttcttttactaagtgttcaaataccagtgaacttaaagaatttgtcaatcctagccttccaagagaagaaaaagaagagaaactagaaacagttaaagtgtctaataatgctgaagaccccaaagatctcatgttaagtggagaaagggttttgcaaactgaaagatctgtagagagtagcagtatttcattggtacctggtactgattatggcactcaggaaagtatctcgttactggaagttagcactctagggaaggcaaaaacagaaccaaataaatgtgtgagtcagtgtgcagcatttgaaaaccccaagggactaattcatggttgttccaaagataatagaaatgacacagaaggctttaagtatccattgggacatgaagttaaccacagtcgggaaacaagcatagaaatggaagaaagtgaacttgatgctcagtatttgcagaatacattcaaggtttcaaagcgccagtcatttgctccgttttcaaatccaggaaatgcagaagaggaatgtgcaacattctctgcccactctgggtccttaaagaaacaaagtccaaaagtcacttttgaatgtgaacaaaaggaagaaaatcaaggaaagaatgagtctaatatcaagcctgtacagacagttaatatcactgcaggctttcctgtggttggtcagaaagataagccagttgataatgccaaatgtagtatcaaaggaggctctaggttttgtctatcatctcagttcagaggcaacgaaactggactcattactccaaataaacatggacttttacaaaacccatatcgtataccaccactttttcccatcaagtcatttgttaaaactaaatgtaagaaaaatctgctagaggaaaactttgaggaacattcaatgtcacctgaaagagaaatgggaaatgagaacattccaagtacagtgagcacaattagccgtaataacattagagaaaatgtttttaaagaagccagctcaagcaatattaatgaagtaggttccagtactaatgaagtgggctccagtattaatgaaataggttccagtgatgaaaacattcaagcagaactaggtagaaacagagggccaaaattgaatgctatgcttagattaggggttttgcaacctgaggtctataaacaaagtcttcctggaagtaattgtaagcatcctgaaataaaaaagcaagaatatgaagaagtagttcagactgttaatacagatttctctccatatctgatttcagataacttagaacagcctatgggaagtagtcatgcatctcaggtttgttctgagacacctgatgacctgttagatgatggtgaaataaaggaagatactagttttgctgaaaatgacattaaggaaagttctgctgtttttagcaaaagcgtccagaaaggagagcttagcaggagtcctagccctttcacccatacacatttggctcagggttaccgaagaggggccaagaaattagagtcctcagaagagaacttatctagtgaggatgaagagcttccctgcttccaacacttgttatttggtaaagtaaacaatataccttctcagtctactaggcatagcaccgttgctaccgagtgtctgtctaagaacacagaggagaatttattatcattgaagaatagcttaaatgactgcagtaaccaggtaatattggcaaaggcatctcaggaacatcaccttagtgaggaaacaaaatgttctgctagcttgttttcttcacagtgcagtgaattggaagacttgactgcaaatacaaacacccaggatcctttcttgattggttcttccaaacaaatgaggcatcagtctgaaagccagggagttggtctgagtgacaaggaattggtttcagatgatgaagaaagaggaacgggcttggaagaaaataatcaagaagagcaaagcatggattcaaacttaggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagcagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactgactgcagccagccacaggtacagagccacaggaccccaagaatgagcttacaaagtggcctttccaggccctgggagctcctctcactcttcagtccttctactgtcctggctactaaatattttatgtacatcagcctgaaaaggacttctggctatgcaagggtcccttaaagattttctgcttgaagtctcccttggaaatctgccatgagcacaaaattatggtaatttttcacctgagaagattttaaaaccatttaaacgccaccaattgagcaagatgctgattcattatttatcagccctattctttctattcaggctgttgttggcttagggctggaagcacagagtggcttggcctcaagagaatagctggtttccctaagtttacttctctaaaaccctgtgttcacaaaggcagagagtcagacccttcaatggaaggagagtgcttgggatcgattatgtgacttaaagtcagaatagtccttgggcagttctcaaatgttggagtggaacattggggaggaaattctgaggcaggtattagaaatgaaaaggaaacttgaaacctgggcatggtggctcacgcctgtaatcccagcactttgggaggccaaggtgggcagatcactggaggtcaggagttcgaaaccagcctggccaacatggtgaaaccccatctctactaaaaatacagaaattagccggtcatggtggtggacacctgtaatcccagctactcaggtggctaaggcaggagaatcacttcagcccgggaggtggaggttgcagtgagccaagatcataccacggcactccagcctgggtgacagtgagactgtggctcaaaaaaaaaaaaaaaaaaaggaaaatgaaactagaagagatttctaaaagtctgagatatatttgctagatttctaaagaatgtgttctaaaacagcagaagattttcaagaaccggtttccaaagacagtcttctaattcctcattagtaataagtaaaatgtttattgttgtagctctggtatataatccattcctcttaaaatataagacctctggcatgaatatttcatatctataaaatgacagatcccaccaggaaggaagctgttgctttctttgaggtgatttttttcctttgctccctgttgctgaaaccatacagcttcataaataattttgcttgctgaaggaagaaaaagtgtttttcataaacccattatccaggactgtttatagctgttggaaggactaggtcttccctagcccccccagtgtgcaagggcagtgaagacttgattgtacaaaatacgttttgtaaatgttgtgctgttaacactgcaaataaacttggtagcaaacacttcca

SEQ ID NO: 4 - DNA, 11954 bp, Homo sapiens
agaggcggagccgctgtggcactgctgcgcctctgctgcgcctcgggtgtcttttgcggcggtgggtcgccgccgggagaagcgtgaggggacagatttgtgaccggcgcggtttttgtcagcttactccggccaaaaaagaactgcacctctggagcggacttatttaccaagcattggaggaatatcgtaggtaaaaatgcctattggatccaaagagaggccaacattttttgaaatttttaagacacgctgcaacaaagcagatttaggaccaataagtcttaattggtttgaagaactttcttcagaagctccaccctataattctgaacctgcagaagaatctgaacataaaaacaacaattacgaaccaaacctatttaaaactccacaaaggaaaccatcttataatcagctggcttcaactccaataatattcaaagagcaagggctgactctgccgctgtaccaatctcctgtaaaagaattagataaattcaaattagacttaggaaggaatgttcccaatagtagacataaaagtcttcgcacagtgaaaactaaaatggatcaagcagatgatgtttcctgtccacttctaaattcttgtcttagtgaaagtcctgttgttctacaatgtacacatgtaacaccacaaagagataagtcagtggtatgtgggagtttgtttcatacaccaaagtttgtgaagggtcgtcagacaccaaaacatatttctgaaagtctaggagctgaggtggatcctgatatgtcttggtcaagttctttagctacaccacccacccttagttctactgtgctcatagtcagaaatgaagaagcatctgaaactgtatttcctcatgatactactgctaatgtgaaaagctatttttccaatcatgatgaaagtctgaagaaaaatgatagatttatcgcttctgtgacagacagtgaaaacacaaatcaaagagaagctgcaagtcatggatttggaaaaacatcagggaattcatttaaagtaaatagctgcaaagaccacattggaaagtcaatgccaaatgtcctagaagatgaagtatatgaaacagttgtagatacctctgaagaagatagtttttcattatgtttttctaaatgtagaacaaaaaatctacaaaaagtaagaactagcaagactaggaaaaaaattttccatgaagcaaacgctgatgaatgtgaaaaatctaaaaaccaagtgaaagaaaaatactcatttgtatctgaagtggaaccaaatgatactgatccattagattcaaatgtagcaaatcagaagccctttgagagtggaagtgacaaaatctccaaggaagttgtaccgtctttggcctgtgaatggtctcaactaaccctttcaggtctaaatggagcccagatggagaaaatacccctattgcatatttcttcatgtgaccaaaatatttcagaaaaagacctattagacacagagaacaaaagaaagaaagattttcttacttcagagaattctttgccacgtatttctagcctaccaaaatcagagaagccattaaatgaggaaacagtggtaaataagagagatgaagagcagcatcttgaatctcatacagactgcattcttgcagtaaagcaggcaatatctggaacttctccagtggcttcttcatttcagggtatcaaaaagtctatattcagaataagagaatcacctaaagagactttcaatgcaagtttttcaggtcatatgactgatccaaactttaaaaaagaaactgaagcctctgaaagtggactggaaatacatactgtttgctcacagaaggaggactccttatgtccaaatttaattgataatggaagctggccagccaccaccacacagaattctgtagctttgaagaatgcaggtttaatatccactttgaaaaagaaaacaaataagtttatttatgctatacatgatgaaacatcttataaaggaaaaaaaataccgaaagaccaaaaatcagaactaattaactgttcagcccagtttgaagcaaatgcttttgaagcaccacttacatttgcaaatgctgattcaggtttattgcattcttctgtgaaaagaagctgttcacagaatgattctgaagaaccaactttgtccttaactagctcttttgggacaattctgaggaaatgttctagaaatgaaacatgttctaataatacagtaatctctcaggatcttgattataaagaagcaaaatgtaataaggaaaaactacagttatttattaccccagaagctgattctctgtcatgcctgcaggaaggacagtgtgaaaatgatccaaaaagcaaaaaagtttcagatataaaagaagaggtcttggctgcagcatgtcacccagtacaacattcaaaagtggaatacagtgatactgactttcaatcccagaaaagtcttttatatgatcatgaaaatgccagcactcttattttaactcctacttccaaggatgttctgtcaaacctagtcatgatttctagaggcaaagaatcatacaaaatgtcagacaagctcaaaggtaacaattatgaatctgatgttgaattaaccaaaaatattcccatggaaaagaatcaagatgtatgtgctttaaatgaaaattataaaaacgttgagctgttgccacctgaaaaatacatgagagtagcatcaccttcaagaaaggtacaattcaaccaaaacacaaatctaagagtaatccaaaaaaatcaagaagaaactacttcaatttcaaaaataactgtcaatccagactctgaagaacttttctcagacaatgagaataattttgtcttccaagtagctaatgaaaggaataatcttgctttaggaaatactaaggaacttcatgaaacagacttgacttgtgtaaacgaacccattttcaagaactctaccatggttttatatggagacacaggtgataaacaagcaacccaagtgtcaattaaaaaagatttggtttatgttcttgcagaggagaacaaaaatagtgtaaagcagcatataaaaatgactctaggtcaagatttaaaatcggacatctccttgaatatagataaaataccagaaaaaaataatgattacatgaacaaatgggcaggactcttaggtccaatttcaaatcacagttttggaggtagcttcagaacagcttcaaataaggaaatcaagctctctgaacataacattaagaagagcaaaatgttcttcaaagatattgaagaacaatatcctactagtttagcttgtgttgaaattgtaaataccttggcattagataatcaaaagaaactgagcaagcctcagtcaattaatactgtatctgcacatttacagagtagtgtagttgtttctgattgtaaaaatagtcatataacccctcagatgttattttccaagcaggattttaattcaaaccataatttaacacctagccaaaaggcagaaattacagaactttctactatattagaagaatcaggaagtcagtttgaatttactcagtttagaaaaccaagctacatattgcagaagagtacatttgaagtgcctgaaaaccagatgactatcttaaagaccacttctgaggaatgcagagatgctgatcttcatgtcataatgaatgccccatcgattggtcaggtagacagcagcaagcaatttgaaggtacagttgaaattaaacggaagtttgctggcctgttgaaaaatgactgtaacaaaagtgcttctggttatttaacagatgaaaatgaagtggggtttaggggcttttattctgctcatggcacaaaactgaatgtttctactgaagctctgcaaaaagctgtgaaactgtttagtgatattgagaatattagtgaggaaacttctgcagaggtacatccaataagtttatcttcaagtaaatgtcatgattctgttgtttcaatgtttaagatagaaaatcataatgataaaactgtaagtgaaaaaaataataaatgccaactgatattacaaaataatattgaaatgactactggcacttttgttgaagaaattactgaaaattacaagagaaatactgaaaatgaagataacaaatatactgctgccagtagaaattctcataacttagaatttgatggcagtgattcaagtaaaaatgatactgtttgtattcataaagatgaaacggacttgctatttactgatcagcacaacatatgtcttaaattatctggccagtttatgaaggagggaaacactcagattaaagaagatttgtcagatttaacttttttggaagttgcgaaagctcaagaagcatgtcatggtaatacttcaaataaagaacagttaactgctactaaaacggagcaaaatataaaagattttgagacttctgatacattttttcagactgcaagtgggaaaaatattagtgtcgccaaagagtcatttaataaaattgtaaatttctttgatcagaaaccagaagaattgcataacttttccttaaattctgaattacattctgacataagaaagaacaaaatggacattctaagttatgaggaaacagacatagttaaacacaaaatactgaaagaaagtgtcccagttggtactggaaatcaactagtgaccttccagggacaacccgaacgtgatgaaaagatcaaagaacctactctattgggttttcatacagctagcgggaaaaaagttaaaattgcaaaggaatctttggacaaagtgaaaaacctttttgatgaaaaagagcaaggtactagtgaaatcaccagttttagccatcaatgggcaaagaccctaaagtacagagaggcctgtaaagaccttgaattagcatgtgagaccattgagatcacagctgccccaaagtgtaaagaaatgcagaattctctcaataatgataaaaaccttgtttctattgagactgtggtgccacctaagctcttaagtgataatttatgtagacaaactgaaaatctcaaaacatcaaaaagtatctttttgaaagttaaagtacatgaaaatgtagaaaaagaaacagcaaaaagtcctgcaacttgttacacaaatcagtccccttattcagtcattgaaaattcagccttagctttttacacaagttgtagtagaaaaacttctgtgagtcagacttcattacttgaagcaaaaaaatggcttagagaaggaatatttgatggtcaaccagaaagaataaatactgcagattatgtaggaaattatttgtatgaaaataattcaaacagtactatagctgaaaatgacaaaaatcatctctccgaaaaacaagatacttatttaagtaacagtagcatgtctaacagctattcctaccattctgatgaggtatataatgattcaggatatctctcaaaaaataaacttgattctggtattgagccagtattgaagaatgttgaagatcaaaaaaacactagtttttccaaagtaatatccaatgtaaaagatgcaaatgcatacccacaaactgtaaatgaagatatttgcgttgaggaacttgtgactagctcttcaccctgcaaaaataaaaatgcagccattaaattgtccatatctaatagtaataattttgaggtagggccacctgcatttaggatagccagtggtaaaatcgtttgtgtttcacatgaaacaattaaaaaagtgaaagacatatttacagacagtttcagtaaagtaattaaggaaaacaacgagaataaatcaaaaatttgccaaacgaaaattatggcaggttgttacgaggcattggatgattcagaggatattcttcataactctctagataatgatgaatgtagcacgcattcacataaggtttttgctgacattcagagtgaagaaattttacaacataaccaaaatatgtctggattggagaaagtttctaaaatatcaccttgtgatgttagtttggaaacttcagatatatgtaaatgtagtatagggaagcttcataagtcagtctcatctgcaaatacttgtgggatttttagcacagcaagtggaaaatctgtccaggtatcagatgcttcattacaaaacgcaagacaagtgttttctgaaatagaagatagtaccaagcaagtcttttccaaagtattgtttaaaagtaacgaacattcagaccagctcacaagagaagaaaatactgctatacgtactccagaacatttaatatcccaaaaaggcttttcatataatgtggtaaattcatctgctttctctggatttagtacagcaagtggaaagcaagtttccattttagaaagttccttacacaaagttaagggagtgttagaggaatttgatttaatcagaactgagcatagtcttcactattcacctacgtctagacaaaatgtatcaaaaatacttcctcgtgttgataagagaaacccagagcactgtgtaaactcagaaatggaaaaaacctgcagtaaagaatttaaattatcaaataacttaaatgttgaaggtggttcttcagaaaataatcactctattaaagtttctccatatctctctcaatttcaacaagacaaacaacagttggtattaggaaccaaagtgtcacttgttgagaacattcatgttttgggaaaagaacaggcttcacctaaaaacgtaaaaatggaaattggtaaaactgaaactttttctgatgttcctgtgaaaacaaatatagaagtttgttctacttactccaaagattcagaaaactactttgaaacagaagcagtagaaattgctaaagcttttatggaagatgatgaactgacagattctaaactgccaagtcatgccacacattctctttttacatgtcccgaaaatgaggaaatggttttgtcaaattcaagaattggaaaaagaagaggagagccccttatcttagtgggagaaccctcaatcaaaagaaacttattaaatgaatttgacaggataatagaaaatcaagaaaaatccttaaaggcttcaaaaagcactccagatggcacaataaaagatcgaagattgtttatgcatcatgtttctttagagccgattacctgtgtaccctttcgcacaactaaggaacgtcaagagatacagaatccaaattttaccgcacctggtcaagaatttctgtctaaatctcatttgtatgaacatctgactttggaaaaatcttcaagcaatttagcagtttcaggacatccattttatcaagtttctgctacaagaaatgaaaaaatgagacacttgattactacaggcagaccaaccaaagtctttgttccaccttttaaaactaaatcacattttcacagagttgaacagtgtgttaggaatattaacttggaggaaaacagacaaaagcaaaacattgatggacatggctctgatgatagtaaaaataagattaatgacaatgagattcatcagtttaacaaaaacaactccaatcaagcagtagctgtaactttcacaaagtgtgaagaagaacctttagatttaattacaagtcttcagaatgccagagatatacaggatatgcgaattaagaagaaacaaaggcaacgcgtctttccacagccaggcagtctgtatcttgcaaaaacatccactctgcctcgaatctctctgaaagcagcagtaggaggccaagttccctctgcgtgttctcataaacagctgtatacgtatggcgtttctaaacattgcataaaaattaacagcaaaaatgcagagtcttttcagtttcacactgaagattattttggtaaggaaagtttatggactggaaaaggaatacagttggctgatggtggatggctcataccctccaatgatggaaaggctggaaaagaagaattttatagggctctgtgtgacactccaggtgtggatccaaagcttatttctagaatttgggtttataatcactatagatggatcatatggaaactggcagctatggaatgtgcctttcctaaggaatttgctaatagatgcctaagcccagaaagggtgcttcttcaactaaaatacagatatgatacggaaattgatagaagcagaagatcggctataaaaaagataatggaaagggatgacacagctgcaaaaacacttgttctctgtgtttctgacataatttcattgagcgcaaatatatctgaaacttctagcaataaaactagtagtgcagatacccaaaaagtggccattattgaacttacagatgggtggtatgctgttaaggcccagttagatcctcccctcttagctgtcttaaagaatggcagactgacagttggtcagaagattattcttcatggagcagaactggtgggctctcctgatgcctgtacacctcttgaagccccagaatctcttatgttaaagatttctgctaacagtactcggcctgctcgctggtataccaaacttggattctttcctgaccctagaccttttcctctgcccttatcatcgcttttcagtgatggaggaaatgttggttgtgttgatgtaattattcaaagagcataccctatacagtggatggagaagacatcatctggattatacatatttcgcaatgaaagagaggaagaaaaggaagcagcaaaatatgtggaggcccaacaaaagagactagaagccttattcactaaaattcaggaggaatttgaagaacatgaagaaaacacaacaaaaccatatttaccatcacgtgcactaacaagacagcaagttcgtgctttgcaagatggtgcagagctttatgaagcagtgaagaatgcagcagacccagcttaccttgagggttatttcagtgaagagcagttaagagccttgaataatcacaggcaaatgttgaatgataagaaacaagctcagatccagttggaaattaggaaggccatggaatctgctgaacaaaaggaacaaggtttatcaagggatgtcacaaccgtgtggaagttgcgtattgtaagctattcaaaaaaagaaaaagattcagttatactgagtatttggcgtccatcatcagatttatattctctgttaacagaaggaaagagatacagaatttatcatcttgcaacttcaaaatctaaaagtaaatctgaaagagctaacatacagttagcagcgacaaaaaaaactcagtatcaacaactaccggtttcagatgaaattttatttcagatttaccagccacgggagccccttcacttcagcaaatttttagatccagactttcagccatcttgttctgaggtggacctaataggatttgtcgtttctgttgtgaaaaaaacaggacttgcccctttcgtctatttgtcagacgaatgttacaatttactggcaataaagttttggatagaccttaatgaggacattattaagcctcatatgttaattgctgcaagcaacctccagtggcgaccagaatccaaatcaggccttcttactttatttgctggagatttttctgtgttttctgctagtccaaaagagggccactttcaagagacattcaacaaaatgaaaaatactgttgagaatattgacatactttgcaatgaagcagaaaacaagcttatgcatatactgcatgcaaatgatcccaagtggtccaccccaactaaagactgtacttcagggccgtacactgctcaaatcattcctggtacaggaaacaagcttctgatgtcttctcctaattgtgagatatattatcaaagtcctttatcactttgtatggccaaaaggaagtctgtttccacacctgtctcagcccagatgacttcaaagtcttgtaaaggggagaaagagattgatgaccaaaagaactgcaaaaagagaagagccttggatttcttgagtagactgcctttacctccacctgttagtcccatttgtacatttgtttctccggctgcacagaaggcatttcagccaccaaggagttgtggcaccaaatacgaaacacccataaagaaaaaagaactgaattctcctcagatgactccatttaaaaaattcaatgaaatttctcttttggaaagtaattcaatagctgacgaagaacttgcattgataaatacccaagctcttttgtctggttcaacaggagaaaaacaatttatatctgtcagtgaatccactaggactgctcccaccagttcagaagattatctcagactgaaacgacgttgtactacatctctgatcaaagaacaggagagttcccaggccagtacggaagaatgtgagaaaaataagcaggacacaattacaactaaaaaatatatctaagcatttgcaaaggcgacaataaattattgacgcttaacctttccagtttataagactggaatataatttcaaaccacacattagtacttatgttgcacaatgagaaaagaaattagtttcaaatttacctcagcgtttgtgtatcgggcaaaaatcgttttgcccgattccgtattggtatacttttgcttcagttgcatatcttaaaactaaatgtaatttattaactaatcaagaaaaacatctttggctgagctcggtggctcatgcctgtaatcccaacactttgagaagctgaggtgggaggagtgcttgaggccaggagttcaagaccagcctgggcaacatagggagacccccatctttacaaagaaaaaaaaaaggggaaaagaaaatcttttaaatctttggatttgatcactacaagtattattttacaagtgaaataaacataccattttcttttagattgtgtcattaaatggaatgaggtctcttagtacagttattttgatgcagataattccttttagtttagctactattttaggggattttttttagaggtaactcactatgaaatagttctccttaatgcaaatatgttggttctgctatagttccatcctgttcaaaagtcaggatgaatatgaagagtggtgtttccttttgagcaattcttcatccttaagtcagcatgattataagaaaaatagaaccctcagtgtaactctaattcctttttactattccagtgtgatctctgaaattaaattacttcaactaaaaattcaaatactttaaatcagaagatttcatagttaatttattttttttttcaacaaaatggtcatccaaactcaaacttgagaaaatatcttgctttcaaattggcactgattctgcctgctttatttttagcgctatcacaggacccagagcctatgcccttttaaacttaccacaaaagcagaagattaattcaatttaagatgatactctcatttgttacgtcctttttttttttttttggagatggagtcttgctttgtcgcccatgctggagtgcagtggcatgatcctggctcactgcagcctccacttcccgggttcacgtaattctcccacctcaagcctccctagtagctgggattacagggacgcaccaccatgcccagctaatttttgcatttttagtagagactgggttttaccatgttggccaagctggtctcaaactcctgatgtcaggtgatccatctgcctcagcctcccaaagtgctgggattataggcgtgagccactgtgcccggccaatatttgttactttcttaggtttaatagagaaaagggataaaacatttctaactgggagttaattgcatggagaaggtcttaaatcagatgttttaatgccttaaatgtctgtataatatcatgttttcaaatctaattataaatacgtttaaagccaagaataaatcttttaaaaaattga

SEQ ID NO: 5 - DNA, 6327 bp, Homo sapiens
agctggcggagggcgttcgtcctgggactgcacttgctcccgtcgggtcgcccggcttcaccggacccgcaggctcccggggcagggccggggccagagctcgcgtgtcggcgggacatgcgctgcgtcgcctctaacctcgggctgtgctctttttccaggtggcccgccggtttctgagccttctgccctgcggggacacggtctgcaccctgcccgcggccacggaccatgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgtaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagcccccgatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagggaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacggtctgagagctccctggctcccacacggttcagataatccctgctgcattttaccctcatcatgcaccactttagccaaattctgtctcctgcatacactccggcatgcatccaacaccaatggctttctagatgagtggccattcatttgcttgctcagttcttagtggcacatcttctgtcttctgttgggaacagccaaagggattccaaggctaaatctttgtaacagctctctttcccccttgctatgttactaagcgtgaggattcccgtagctcttcacagctgaactcagtctatgggttggggctcagataactctgtgcatttaagctacttgtagagacccaggcctggagagtagacattttgcctctgataagcactttttaaatggctctaagaataagccacagcaaagaatttaaagtggctcctttaattggtgacttggagaaagctaggtcaagggtttattatagcaccctcttgtattcctatggcaatgcatccttttatgaaagtggtacaccttaaagcttttatatgactgtagcagagtatctggtgattgtcaattcattccccctataggaatacaaggggcacacagggaaggcagatcccctagttggcaagactattttaacttgatacactgcagattcagatgtgctgaaagctctgcctctggctttccggtcatgggttccagttaattcatgcctcccatggacctatggagagcagcaagttgatcttagttaagtctccctatatgagggataagttcctgatttttgtttttatttttgtgttacaaaagaaagccctccctccctgaacttgcagtaaggtcagcttcaggacctgttccagtgggcactgtacttggatcttcccggcgtgtgtgtgccttacacaggggtgaactgttcactgtggtgatgcatgatgagggtaaatggtagttgaaaggagcaggggccctggtgttgcatttagccctggggcatggagctgaacagtacttgtgcaggattgttgtggctactagagaacaagagggaaagtagggcagaaactggatacagttctgaggcacagccagacttgctcagggtggccctgccacaggctgcagctacctaggaacattccttgcagaccccgcattgccctttgggggtgccctgggatccctggggtagtccagctcttcttcatttcccagcgtggccctggttggaagaagcagctgtcacagctgctgtagacagctgtgttcctacaattggcccagcaccctggggcacgggagaagggtggggaccgttgctgtcactactcaggctgactggggcctggtcagattacgtatgcccttggtggtttagagataatccaaaatcagggtttggtttggggaagaaaatcctcccccttcctcccccgccccgttccctaccgcctccactcctgccagctcatttccttcaatttcctttgacctataggctaaaaaagaaaggctcattccagccacagggcagccttccctgggcctttgcttctctagcacaattatgggttacttcctttttcttaacaaaaaagaatgtttgatttcctctgggtgaccttattgtctgtaattgaaaccctattgagaggtgatgtctgtgttagccaatgacccaggtgagctgctcgggcttctcttggtatgtcttgtttggaaaagtggatttcattcatttctgattgtccagttaagtgatcaccaaaggactgagaatctgggagggcaaaaaaaaaaaaaaagtttttatgtgcacttaaatttggggacaattttatgtatctgtgttaaggatatgtttaagaacataattcttttgttgctgtttgtttaagaagcaccttagtttgtttaagaagcaccttatatagtataatatatatttttttgaaattacattgcttgtttatcagacaattgaatgtagtaattctgttctggatttaatttgactgggttaacatgcaaaaaccaaggaaaaatatttagtttttttttttttttttgtatacttttcaagctaccttgtcatgtatacagtcatttatgcctaaagcctggtgattattcatttaaatgaagatcacatttcatatcaacttttgtatccacagtagacaaaatagcactaatccagatgcctattgttggatactgaatgacagacaatcttatgtagcaaagattatgcctgaaaaggaaaattattcagggcagctaattttgcttttaccaaaatatcagtagtaatatttttggacagtagctaatgggtcagtgggttctttttaatgtttatacttagattttcttttaaaaaaattaaaataaaacaaaaaaaaatttctaggactagacgatgtaataccagctaaagccaaacaattatacagtggaaggttttacattattcatccaatgtgtttctattcatgttaagatactactacatttgaagtgggcagagaacatcagatgattgaaatgttcgcccaggggtctccagcaactttggaaatctctttgtatttttacttgaagtgccactaatggacagcagatattttctggctgatgttggtattgggtgtaggaacatgatttaaaaaaaaactcttgcctctgctttcccccactctgaggcaagttaaaatgtaaaagatgtgatttatctggggggctcaggtatggtggggaagtggattcaggaatctggggaatggcaaatatattaagaagagtattgaaagtatttggaggaaaatggttaattctgggtgtgcaccagggttcagtagagtccacttctgccctggagaccacaaatcaactagctccatttacagccatttctaaaatggcagcttcagttctagagaagaaagaacaacatcagcagtaaagtccatggaatagctagtggtctgtgtttcttttcgccattgcctagcttgccgtaatgattctataatgccatcatgcagcaattatgagaggctaggtcatccaaagagaagaccctatcaatgtaggttgcaaaatctaacccctaaggaagtgcagtctttgatttgatttccctagtaaccttgcagatatgtttaaccaagccatagcccatgccttttgagggctgaacaaataagggacttactgataatttacttttgatcacattaaggtgttctcaccttgaaatcttatacactgaaatggccattgatttaggccactggcttagagtactccttcccctgcatgacactgattacaaatactttcctattcatactttccaattatgagatggactgtgggtactgggagtgatcactaacaccatagtaatgtctaatattcacaggcagatctgcttggggaagctagttatgtgaaaggcaaatagagtcatacagtagctcaaaaggcaaccataattctctttggtgcaggtcttgggagcgtgatctagattacactgcaccattcccaagttaatcccctgaaaacttactctcaactggagcaaatgaactttggtcccaaatatccatcttttcagtagcgttaattatgctctgtttccaactgcatttcctttccaattgaattaaagtgtggcctcgtttttagtcatttaaaattgttttctaagtaattgctgcctctattatggcacttcaattttgcactgtcttttgagattcaagaaaaatttctattcttttttttgcatccaattgtgcctgaacttttaaaatatgtaaatgctgccatgttccaaacccatcgtcagtgtgtgtgtttagagctgtgcaccctagaaacaacatattgtcccatgagcaggtgcctgagacacagacccctttgcattcacagagaggtcattggttatagagacttgaattaataagtgacattatgccagtttctgttctctcacaggtgataaacaatgctttttgtgcactacatactcttcagtgtagagctcttgttttatgggaaaaggctcaaatgccaaattgtgtttgatggattaatatgcccttttgccgatgcatactattactgatgtgactcggttttgtcgcagctttgctttgtttaatgaaacacacttgtaaacctcttttgcactttgaaaaagaatccagcgggatgctcgagcacctgtaaacaattttctcaacctatttgatgttcaaataaagaattaaactaaa

SEQ ID NO: 6 - DNA, 12373 bp, Homo sapiens
acttccggtggggtgtcatggcggcgtctcggactgtgatggctgtggggagacggcgctagtggggagagcgaccaagaggccccctcccctccccgggtccccttcccctatccccctccccccagcctccttgccaacgccccctttccctctccccctcccgctcggcgctgaccccccatccccacccccgtgggaacactgggagcctgcactccacagaccctctccttgcctcttccctcacctcagcctccgctccccgccctcttcccggcccagggcgccggcccacccttccctccgccgccccccggccgcggggaggacatggccgcgcacaggccggtggaatgggtccaggccgtggtcagccgcttcgacgagcagcttccaataaaaacaggacagcagaacacacataccaaagtcagtactgagcacaacaaggaatgtctaatcaatatttccaaatacaagttttctttggttataagcggcctcactactattttaaagaatgttaacaatatgagaatatttggagaagctgctgaaaaaaatttatatctctctcagttgattatattggatacactggaaaaatgtcttgctgggcaaccaaaggacacaatgagattagatgaaacgatgctggtcaaacagttgctgccagaaatctgccattttcttcacacctgtcgtgaaggaaaccagcatgcagctgaacttcggaattctgcctctggggttttattttctctcagctgcaacaacttcaatgcagtctttagtcgcatttctaccaggttacaggaattaactgtttgttcagaagacaatgttgatgttcatgatatagaattgttacagtatatcaatgtggattgtgcaaaattaaaacgactcctgaaggaaacagcatttaaatttaaagccctaaagaaggttgcgcagttagcagttataaatagcctggaaaaggcattttggaactgggtagaaaattatccagatgaatttacaaaactgtaccagatcccacagactgatatggctgaatgtgcagaaaagctatttgacttggtggatggttttgctgaaagcaccaaacgtaaagcagcagtttggccactacaaatcattctccttatcttgtgtccagaaataatccaggatatatccaaagacgtggttgatgaaaacaacatgaataagaagttatttctggacagtctacgaaaagctcttgctggccatggaggaagtaggcagctgacagaaagtgctgcaattgcctgtgtcaaactgtgtaaagcaagtacttacatcaattgggaagataactctgtcattttcctacttgttcagtccatggtggttgatcttaagaacctgctttttaatccaagtaagccattctcaagaggcagtcagcctgcagatgtggatctaatgattgactgccttgtttcttgctttcgtataagccctcacaacaaccaacactttaagatctgcctggctcagaattcaccttctacatttcactatgtgctggtaaattcactccatcgaatcatcaccaattccgcattggattggtggcctaagattgatgctgtgtattgtcactcggttgaacttcgaaatatgtttggtgaaacacttcataaagcagtgcaaggttgtggagcacacccagcaatacgaatggcaccgagtcttacatttaaagaaaaagtaacaagccttaaatttaaagaaaaacctacagacctggagacaagaagctataagtatcttctcttgtccatggtgaaactaattcatgcagatccaaagctcttgctttgtaatccaagaaaacaggggcccgaaacccaaggcagtacagcagaattaattacagggctcgtccaactggtccctcagtcacacatgccagagattgctcaggaagcaatggaggctctgctggttcttcatcagttagatagcattgatttgtggaatcctgatgctcctgtagaaacattttgggagattagctcacaaatgcttttttacatctgcaagaaattaactagtcatcaaatgcttagtagcacagaaattctcaagtggttgcgggaaatattgatctgcaggaataaatttcttcttaaaaataagcaggcagatagaagttcctgtcactttctccttttttacggggtaggatgtgatattccttctagtggaaataccagtcaaatgtccatggatcatgaagaattactacgtactcctggagcctctctccggaagggaaaagggaactcctctatggatagtgcagcaggatgcagcggaacccccccgatttgccgacaagcccagaccaaactagaagtggccctgtacatgtttctgtggaaccctgacactgaagctgttctggttgccatgtcctgtttccgccacctctgtgaggaagcagatatccggtgtggggtggatgaagtgtcagtgcataacctcttgcccaactataacacattcatggagtttgcctctgtcagcaatatgatgtcaacaggaagagcagcacttcagaaaagagtgatggcactgctgaggcgcattgagcatcccactgcaggaaacactgaggcttgggaagatacacatgcaaaatgggaacaagcaacaaagctaatccttaactatccaaaagccaaaatggaagatggccaggctgctgaaagccttcacaagaccattgttaagaggcgaatgtcccatgtgagtggaggaggatccatagatttgtctgacacagactccctacaggaatggatcaacatgactggcttcctttgtgcccttgggggagtgtgcctccagcagagaagcaattctggcctggcaacctatagcccacccatgggtccagtcagtgaacgtaagggttctatgatttcagtgatgtcttcagagggaaacgcagatacacctgtcagcaaatttatggatcggctgttgtccttaatggtgtgtaaccatgagaaagtgggacttcaaatacggaccaatgttaaggatctggtgggtctagaattgagtcctgctctgtatccaatgctatttaacaaattgaagaataccatcagcaagttttttgactcccaaggacaggttttattgactgataccaatactcaatttgtagaacaaaccatagctataatgaagaacttgctagataatcatactgaaggcagctctgaacatctagggcaagctagcattgaaacaatgatgttaaatctggtcaggtatgttcgtgtgcttgggaatatggtccatgcaattcaaataaaaacgaaactgtgtcaattagttgaagtaatgatggcaaggagagatgacctctcattttgccaagagatgaaatttaggaataagatggtagaatacctgacagactgggttatgggaacatcaaaccaagcagcagatgatgatgtaaaatgtcttacaagagatttggaccaggcaagcatggaagcagtagtttcacttctagctggtctccctctgcagcctgaagaaggagatggtgtggaattgatggaagccaaatcacagttatttcttaaatacttcacattatttatgaaccttttgaatgactgcagtgaagttgaagatgaaagtgcgcaaacaggtggcaggaaacgtggcatgtctcggaggctggcatcactgaggcactgtacggtccttgcaatgtcaaacttactcaatgccaacgtagacagtggtctcatgcactccataggcttaggttaccacaaggatctccagacaagagctacatttatggaagttctgacaaaaatccttcaacaaggcacagaatttgacacacttgcagaaacagtattggctgatcggtttgagagattggtggaactggtcacaatgatgggtgatcaaggagaactccctatagcgatggctctggccaatgtggttccttgttctcagtgggatgaactagctcgagttctggttactctgtttgattctcggcatttactctaccaactgctctggaacatgttttctaaagaagtagaattggcagactccatgcagactctcttccgaggcaacagcttggccagtaaaataatgacattctgtttcaaggtatatggtgctacctatctacaaaaactcctggatcctttattacgaattgtgatcacatcctctgattggcaacatgttagctttgaagtggatcctaccaggttagaaccatcagagagccttgaggaaaaccagcggaacctccttcagatgactgaaaagttcttccatgccatcatcagttcctcctcagaattcccccctcaacttcgaagtgtgtgccactgtttataccaggcaacttgccactccctactgaataaagctacagtaaaagaaaaaaaggaaaacaaaaaatcagtggttagccagcgtttccctcagaacagcatcggtgcagtaggaagtgccatgttcctcagatttatcaatcctgccattgtctcaccgtatgaagcagggattttagataaaaagccaccacctagaatcgaaaggggcttgaagttaatgtcaaagatacttcagagtattgccaatcatgttctcttcacaaaagaagaacatatgcggcctttcaatgattttgtgaaaagcaactttgatgcagcacgcaggtttttccttgatatagcatctgattgtcctacaagtgatgcagtaaatcatagtctttccttcataagtgacggcaatgtgcttgctttacatcgtctactctggaacaatcaggagaaaattgggcagtatctttccagcaacagggatcataaagctgttggaagacgaccttttgataagatggcaacacttcttgcatacctgggtcctccagagcacaaacctgtggcagatacacactggtccagccttaaccttaccagttcaaagtttgaggaatttatgactaggcatcaggtacatgaaaaagaagaattcaaggctttgaaaacgttaagtattttctaccaagctgggacttccaaagctgggaatcctattttttattatgttgcacggaggttcaaaactggtcaaatcaatggtgatttgctgatataccatgtcttactgactttaaagccatattatgcaaagccatatgaaattgtagtggaccttacccataccgggcctagcaatcgctttaaaacagactttctctctaagtggtttgttgtttttcctggctttgcttacgacaacgtctccgcagtctatatctataactgtaactcctgggtcagggagtacaccaagtatcatgagcggctgctgactggcctcaaaggtagcaaaaggcttgttttcatagactgtcctgggaaactggctgagcacatagagcatgaacaacagaaactacctgctgccaccttggctttagaagaggacctgaaggtattccacaatgctctcaagctagctcacaaagacaccaaagtttctattaaagttggttctactgctgtccaagtaacttcagcagagcgaacaaaagtcctagggcaatcagtctttctaaatgacatttattatgcttcggaaattgaagaaatctgcctagtagatgagaaccagttcaccttaaccattgcaaaccagggcacgccgctcaccttcatgcaccaggagtgtgaagccattgtccagtctatcattcatatccggacccgctgggaactgtcacagcccgactctatcccccaacacaccaagattcggccaaaagatgtccctgggacactgctcaatatcgcattacttaatttaggcagttctgacccgagtttacggtcagctgcctataatcttctgtgtgccttaacttgtacctttaatttaaaaatcgagggccagttactagagacatcaggtttatgtatccctgccaacaacaccctctttattgtctctattagtaagacactggcagccaatgagccacacctcacgttagaatttttggaagagtgtatttctggatttagcaaatctagtattgaattgaaacacctttgtttggaatacatgactccatggctgtcaaatctagttcgtttttgcaagcataatgatgatgccaaacgacaaagagttactgctattcttgacaagctgataacaatgaccatcaatgaaaaacagatgtacccatctattcaagcaaaaatatggggaagccttgggcagattacagatctgcttgatgttgtactagacagtttcatcaaaaccagtgcaacaggtggcttgggatcaataaaagctgaggtgatggcagatactgctgtagctttggcttctggaaatgtgaaattggtttcaagcaaggttattggaaggatgtgcaaaataattgacaagacatgcttatctccaactcctactttagaacaacatcttatgtgggatgatattgctattttagcacgctacatgctgatgctgtccttcaacaattcccttgatgtggcagctcatcttccctacctcttccacgttgttactttcttagtagccacaggtccgctctcccttagagcttccacacatggactggtcattaatatcattcactctctgtgtacttgttcacagcttcattttagtgaagagaccaagcaagttttgagactcagtctgacagagttctcattacccaaattttacttgctgtttggcattagcaaagtcaagtcagctgctgtcattgccttccgttccagttaccgggacaggtcattctctcctggctcctatgagagagagacttttgctttgacatccttggaaacagtcacagaagctttgttggagatcatggaggcatgcatgagagatattccaacgtgcaagtggctggaccagtggacagaactagctcaaagatttgcattccaatataatccatccctgcaaccaagagctcttgttgtctttgggtgtattagcaaacgagtgtctcatgggcagataaagcagataatccgtattcttagcaaggcacttgagagttgcttaaaaggacctgacacttacaacagtcaagttctgatagaagctacagtaatagcactaaccaaattacagccacttcttaataaggactcgcctctgcacaaagccctcttttgggtagctgtggctgtgctgcagcttgatgaggtcaacttgtattcagcaggtaccgcacttcttgaacaaaacctgcatactttagatagtctccgtatattcaatgacaagagtccagaggaagtatttatggcaatccggaatcctctggagtggcactgcaagcaaatggatcattttgttggactcaatttcaactctaactttaactttgcattggttggacaccttttaaaagggtacaggcatccttcacctgctattgttgcaagaacagtcagaattttacatacactactaactctggttaacaaacacagaaattgtgacaaatttgaagtgaatacacagagcgtggcctacttagcagctttacttacagtgtctgaagaagttcgaagtcgctgcagcctaaaacatagaaagtcacttcttcttactgatatttcaatggaaaatgttcctatggatacatatcccattcatcatggtgacccttcctataggacactaaaggagactcagccatggtcctctcccaaaggttctgaaggataccttgcagccacctatccaactgtcggccagaccagtccccgagccaggaaatccatgagcctggacatggggcaaccttctcaggccaacactaagaagttgcttggaacaaggaaaagttttgatcacttgatatcagacacaaaggctcctaaaaggcaagaaatggaatcagggatcacaacaccccccaaaatgaggagagtagcagaaactgattatgaaatggaaactcagaggatttcctcatcacaacagcacccacatttacgtaaagtttcagtgtctgaatcaaatgttctcttggatgaagaagtacttactgatccgaagatccaggcgctgcttcttactgttctagctacactggtaaaatataccacagatgagtttgatcaacgaattctttatgaatacttagcagaggccagtgttgtgtttcccaaagtctttcctgttgtgcataatttgttggactctaagatcaacaccctgttatcattgtgccaagatccaaatttgttaaatccaatccatggaattgtgcagagtgtggtgtaccatgaagaatccccaccacaataccaaacatcttacctgcaaagttttggttttaatggcttgtggcggtttgcaggaccgttttcaaagcaaacacaaattccagactatgctgagcttattgttaagtttcttgatgccttgattgacacgtacctgcctggaattgatgaagaaaccagtgaagaatccctcctgactcccacatctccttaccctcctgcactgcagagccagcttagtatcactgccaaccttaacctttctaattccatgacctcacttgcaacttcccagcattccccaggaatcgacaaggagaacgttgaactctcccctaccactggccactgtaacagtggacgaactcgccacggatccgcaagccaagtgcagaagcaaagaagcgctggcagtttcaaacgtaatagcattaagaagatcgtgtgaagcttgcttgctttcttttttaaaatcaacttaacatgggctcttcactagtgaccccttccctgtccttgccctttccccccatgttgtaatgctgcacttcctgttttataatgaacccatccggtttgccatgttgccagatgatcaactcttcgaagccttgcctaaatttaatgctgccttttctttaactttttttcttctacttttggcgtgtatctggtatatgtaagtgttcagaacaactgcaaagaaagtgggaggtcaggaaacttttaactgagaaatctcaattgtaagagaggatgaattcttgaatactgctactactggccagtgatgaaagccatttgcacagagctctgccttctgtggttttcccttcttcatcctacagagtaaagtgttagtcctatttatacatttttcaagatacaagtttatgagagaaatagtattataaccccagtatgtttaatcttttagctgtggactttttttttaaccgtacaaaactgaaagaaccatagaggtcaagcctcagtgacttgacaccataaagccacagacaaggtacttgggggggagggcagggaaatttcatattttatagtggattcttaagaaatactaacacttgagtattagcaataattacaggaaaataagtgcgaccacatatatcttaacattactgaattaaaactatggcttctaagtccttatccaaactcagtcatccaaactagtttatttttttctccagttgattatcttttaatttttaattttgctaaaggtggtttttttgtgttttgttttttgtaaaccaaaactatactaagtatagtaattatatatatatatatattttttcccctccccctcttctttcctaactaattctgagcagggtaatcagtgaacaaagtgttgaaaattgttcccagaaggtaattttcatagatgtttgcattagctccatagcaaaatggaatggtacgtgacatttagggtagctgatatttttattttgttaaataatttccaagaatagagtatggtgtatattataaatttctttgataagatgtattttgaatgtcttttaatcttcctcctcctctccaaaaaaatcagaaacctctttaagaaaacatgtaggttatatatgctagaattgcatttaatcactgtgaaaagactggtcagcctgcattagtatgacagtaggggggctgttagaattgctgctatactggtggtatggattatcatggcattggaattttcatagtaatgcagatccaatttctttgtggtacctgcagtttacaaaataatttgacttcagtgagcatattggtatctggatgttccaatttagaactaaaccatatttattacaaaaagatattaatccctctactcccaggttccctttatatgttaagatataatggctttgaggggggaaaaaataaacctaggggagaggggagtttcctgtagtgctgtttcattagaggatttcagtaaattaaattccacagctaattcaataaataatggtacatttaagtgttctgattttaataatatatttcacatttatccacacagtaacaatgtaatatgttaatgtaaataaaattggttttgatactcagaaataacaagaatttaattttttaaatttgtttacagtcctgggaaaagtaagaattatttgccaaaataagaggaaagaaaaccttagtattattaatgagtttaccatagaattgttggaaatactgaagacaggtgcaatttactaaacttttgtttttaaactattgtagaggctgcattagaagaaaatgtttataatgacagagcaactatgactatataaaaaagctgaaattagaactgtgtttagaaatagatcagtaacccagtgccaaggatgccaagctgccaccatggtcttggctctcccacaacccagtgtttctggggtaagtttcacagtttctaggccctggaatagcaggcagtgtaagcctttgataactttagttcgatgtttttcttgtttttgtttgttggtttggtgcatatgatagtgggtgttatgctattttgctcttcccatcaaaataaagaaacttccagaggtttactgttaaaaatactgatatttccataaacgggtttaccaagggtgtagtatttcataccgcctgaaatgatcagcattggcacaaatcaaaattcagccgcctttgaaatgcaaaaatacctttgactagtaagtacatcctaggagtttgaaaacttaactaaggtttaaaatttaccttgtttaaagaacttctgacttttgaggaaaatctagctttccaagtaactaaaatgtacatgagataaacctctcaccactatgtgtcccttgagaaatgcaacacttttttagtcttcatacttgtaatctataaaagaaattctgaagtttagaccaagttgcccatttctgcgtaattgacataagttctgttaaaaatattataagtaattcgtttcggtttgtagatgtttcccctgacttgttaaagaggaaaccaggaactcagtcatgtttttgtcctggataatctacctgttatgccagtactcccatccgaggggcatgcccttagttgcccagatggagatgcagttcagtagatttggggcaaagtggctacagctctgtcttccattcactcaacacctgttcatgactgagccaggtgcccaggacacatcctaaacagtcagcttctatcctgtgtcctagttggggagacagagtgccagccagcaaccctcccaggtttgtaggttttaggggttttcagttttgtttgggttttttgttttttgtttttgtttctacatccttccccgactcccaggcataatgaggcatgtcttactcaatgttatgcaatggatttaggcaaaaattcattcttagtgtcagccacacaattttttttaatgcagtatattcacctgtaaatagtttgtgtaaaatttgacaaaaaaagtatatttactatactgtaaatatatgtgatgatatattgtattattttgcttttttgtaaagcagttagttgctgcacatggataacaacaaaaatttgattattctcgtgttagtattgttaacttctttttgcgactgcgttacatcatttaaagaaaatgctgtgtattgtaaacttaaattgtatatgataacttactgtcctttccatccgggcctaaactttggcagttcctttgtctacaaccttgttaatactgtaaacagttgtacgccagcaggaaaaatactgcccaacagacaaaatcgatcattgtaggggaaaatcatagaaatccatttcagatctttattgttcctcaccccattttcctccttgtgtatgtacttcccccacccccctttttttaagtaaaatgtaaattcaatctgctctaa

SEQ ID NO: 7 - DNA, 2889 bp, Homo sapiens
acttccggtggggtgtcatggcggcgtctcggactgtgatggctgtggggagacggcgctagtggggagagcgaccaagaggccccctcccctccccgggtccccttcccctatccccctccccccagcctccttgccaacgccccctttccctctccccctcccgctcggcgctgaccccccatccccacccccgtgggaacactgggagcctgcactccacagaccctctccttgcctcttccctcacctcagcctccgctccccgccctcttcccggcccagggcgccggcccacccttccctccgccgccccccggccgcggggaggacatggccgcgcacaggccggtggaatgggtccaggccgtggtcagccgcttcgacgagcagcttccaataaaaacaggacagcagaacacacataccaaagtcagtactgagcacaacaaggaatgtctaatcaatatttccaaatacaagttttctttggttataagcggcctcactactattttaaagaatgttaacaatatgagaatatttggagaagctgctgaaaaaaatttatatctctctcagttgattatattggatacactggaaaaatgtcttgctgggcaaccaaaggacacaatgagattagatgaaacgatgctggtcaaacagttgctgccagaaatctgccattttcttcacacctgtcgtgaaggaaaccagcatgcagctgaacttcggaattctgcctctggggttttattttctctcagctgcaacaacttcaatgcagtctttagtcgcatttctaccaggttacaggaattaactgtttgttcagaagacaatgttgatgttcatgatatagaattgttacagtatatcaatgtggattgtgcaaaattaaaacgactcctgaaggaaacagcatttaaatttaaagccctaaagaaggttgcgcagttagcagttataaatagcctggaaaaggcattttggaactgggtagaaaattatccagatgaatttacaaaactgtaccagatcccacagactgatatggctgaatgtgcagaaaagctatttgacttggtggatggttttgctgaaagcaccaaacgtaaagcagcagtttggccactacaaatcattctccttatcttgtgtccagaaataatccaggatatatccaaagacgtggttgatgaaaacaacatgaataagaagttatttctggacagtctacgaaaagctcttgctggccatggaggaagtaggcagctgacagaaagtgctgcaattgcctgtgtcaaactgtgtaaagcaagtacttacatcaattgggaagataactctgtcattttcctacttgttcagtccatggtggttgatcttaagaacctgctttttaatccaagtaagccattctcaagaggcagtcagcctgcagatgtggatctaatgattgactgccttgtttcttgctttcgtataagccctcacaacaaccaacactttaagatctgcctggctcagaattcaccttctacatttcactatgtgctggtaaattcactccatcgaatcatcaccaattccgcattggattggtggcctaagattgatgctgtgtattgtcactcggttgaacttcgaaatatgtttggtgaaacacttcataaagcagtgcaaggttgtggagcacacccagcaatacgaatggcaccgagtcttacatttaaagaaaaagtaacaagccttaaatttaaagaaaaacctacagacctggagacaagaagctataagtatcttctcttgtccatggtgaaactaattcatgcagatccaaagctcttgctttgtaatccaagaaaacaggggcccgaaacccaaggcagtacagcagaattaattacagggctcgtccaactggtccctcagtcacacatgccagagattgctcaggaagcaatggaggctctgctggttcttcatcagttagatagcattgatttgtggaatcctgatgctcctgtagaaacattttgggagattaggtatatgtacttttattttttaaattcaacttttaaattttattttgtatttttgtcttgaaatattaactctgtagtacttagtacattgtaaaacttacacttccaaaggttttatggttttgtattttatttgacttcaaattattagaatttcttgttttaactgtaagaaaagtatcacagcaatttagaaaataaattttaagaatagtgctaaattttgtcaccctaacataagtactgttgtttggtatattacttttttcagatttcaatgtggttactactgtatttttaatagattttcatagttataagcctagaatgataaaattttgtaacaatactgttttttcagttttttgaactatgatctttcataaactttctgtaataccaatgctttcgatgaatgaattaataatggacacctgcttagaagaaaaaaatgtatgcagaattttgtggtctgcttcctagattatacaaatcattacattttaatgagcatgaagtcaccacacggaggaaaatgtaaatgtgtaaacctcaagtttgccattatcttataagaatgggtgtgctaagttacttggcagctgaattaaaccttactctagagtagtgctgtccactagtaatataatgagaaccccatatgttaaaaacttttttacttactacattaaagagtaaaaagaagcaggtaaaattaattgtaattatgttttgtttaacccggtgtatattatctataatttcaacatgtaatcaatataaaagttattaatgacatttcattctcttttttttaataaagtcttcaaacttt

SEQ ID NO: 8 - DNA, 4768 bp, Homo sapiens
agttgccgggcgggggagggcgcgtccggtttttctcaggggacgttgaaattatttttgtaacgggagtcgggagaggacggggcgtgccccgacgtgcgcgcgcgtcgtcctccccggcgctcctccacagctcgctggctcccgccgcggaaaggcgtcatgccgcccaaaaccccccgaaaaacggccgccaccgccgccgctgccgccgcggaacccccggcaccgccgccgccgccccctcctgaggaggacccagagcaggacagcggcccggaggacctgcctctcgtcaggcttgagtttgaagaaacagaagaacctgattttactgcattatgtcagaaattaaagataccagatcatgtcagagagagagcttggttaacttgggagaaagtttcatctgtggatggagtattgggaggttatattcaaaagaaaaaggaactgtggggaatctgtatctttattgcagcagttgacctagatgagatgtcgttcacttttactgagctacagaaaaacatagaaatcagtgtccataaattctttaacttactaaaagaaattgataccagtaccaaagttgataatgctatgtcaagactgttgaagaagtatgatgtattgtttgcactcttcagcaaattggaaaggacatgtgaacttatatatttgacacaacccagcagttcgatatctactgaaataaattctgcattggtgctaaaagtttcttggatcacatttttattagctaaaggggaagtattacaaatggaagatgatctggtgatttcatttcagttaatgctatgtgtccttgactattttattaaactctcacctcccatgttgctcaaagaaccatataaaacagctgttatacccattaatggttcacctcgaacacccaggcgaggtcagaacaggagtgcacggatagcaaaacaactagaaaatgatacaagaattattgaagttctctgtaaagaacatgaatgtaatatagatgaggtgaaaaatgtttatttcaaaaattttataccttttatgaattctcttggacttgtaacatctaatggacttccagaggttgaaaatctttctaaacgatacgaagaaatttatcttaaaaataaagatctagatgcaagattatttttggatcatgataaaactcttcagactgattctatagacagttttgaaacacagagaacaccacgaaaaagtaaccttgatgaagaggtgaatgtaattcctccacacactccagttaggactgttatgaacactatccaacaattaatgatgattttaaattcagcaagtgatcaaccttcagaaaatctgatttcctattttaacaactgcacagtgaatccaaaagaaagtatactgaaaagagtgaaggatataggatacatctttaaagagaaatttgctaaagctgtgggacagggttgtgtcgaaattggatcacagcgatacaaacttggagttcgcttgtattaccgagtaatggaatccatgcttaaatcagaagaagaacgattatccattcaaaattttagcaaacttctgaatgacaacatttttcatatgtctttattggcgtgcgctcttgaggttgtaatggccacatatagcagaagtacatctcagaatcttgattctggaacagatttgtctttcccatggattctgaatgtgcttaatttaaaagcctttgatttttacaaagtgatcgaaagttttatcaaagcagaaggcaacttgacaagagaaatgataaaacatttagaacgatgtgaacatcgaatcatggaatcccttgcatggctctcagattcacctttatttgatcttattaaacaatcaaaggaccgagaaggaccaactgatcaccttgaatctgcttgtcctcttaatcttcctctccagaataatcacactgcagcagatatgtatctttctcctgtaagatctccaaagaaaaaaggttcaactacgcgtgtaaattctactgcaaatgcagagacacaagcaacctcagccttccagacccagaagccattgaaatctacctctctttcactgttttataaaaaagtgtatcggctagcctatctccggctaaatacactttgtgaacgccttctgtctgagcacccagaattagaacatatcatctggacccttttccagcacaccctgcagaatgagtatgaactcatgagagacaggcatttggaccaaattatgatgtgttccatgtatggcatatgcaaagtgaagaatatagaccttaaattcaaaatcattgtaacagcatacaaggatcttcctcatgctgttcaggagacattcaaacgtgttttgatcaaagaagaggagtatgattctattatagtattctataactcggtcttcatgcagagactgaaaacaaatattttgcagtatgcttccaccaggccccctaccttgtcaccaatacctcacattcctcgaagcccttacaagtttcctagttcacccttacggattcctggagggaacatctatatttcacccctgaagagtccatataaaatttcagaaggtctgccaacaccaacaaaaatgactccaagatcaagaatcttagtatcaattggtgaatcattcgggacttctgagaagttccagaaaataaatcagatggtatgtaacagcgaccgtgtgctcaaaagaagtgctgaaggaagcaaccctcctaaaccactgaaaaaactacgctttgatattgaaggatcagatgaagcagatggaagtaaacatctcccaggagagtccaaatttcagcagaaactggcagaaatgacttctactcgaacacgaatgcaaaagcagaaaatgaatgatagcatggatacctcaaacaaggaagagaaatgaggatctcaggaccttggtggacactgtgtacacctctggattcattgtctctcacagatgtgactgtataactttcccaggttctgtttatggccacatttaatatcttcagctctttttgtggatataaaatgtgcagatgcaattgtttgggtgattcctaagccacttgaaatgttagtcattgttatttatacaagattgaaaatcttgtgtaaatcctgccatttaaaaagttgtagcagattgtttcctcttccaaagtaaaattgctgtgctttatggatagtaagaatggccctagagtgggagtcctgataacccaggcctgtctgactactttgccttcttttgtagcatataggtgatgtttgctcttgtttttattaatttatatgtatatttttttaatttaacatgaacacccttagaaaatgtgtcctatctatcttccaaatgcaatttgattgactgcccattcaccaaaattatcctgaactcttctgcaaaaatggatattattagaaattagaaaaaaattactaattttacacattagattttattttactattggaatctgatatactgtgtgcttgttttataaaattttgcttttaattaaataaaagctggaagcaaagtataaccatatgatactatcatactactgaaacagatttcatacctcagaatgtaaaagaacttactgattattttcttcatccaacttatgtttttaaatgaggattattgatagtactcttggtttttataccattcagatcactgaatttataaagtacccatctagtacttgaaaaagtaaagtgttctgccagatcttaggtatagaggaccctaacacagtatatcccaagtgcactttctaatgtttctgggtcctgaagaattaagatacaaattaattttactccataaacagactgttaattataggagccttaatttttttttcatagagatttgtctaattgcatctcaaaattattctgccctccttaatttgggaaggtttgtgttttctctggaatggtacatgtcttccatgtatcttttgaactggcaattgtctatttatcttttatttttttaagtcagtatggtctaacactggcatgttcaaagccacattatttctagtccaaaattacaagtaatcaagggtcattatgggttaggcattaatgtttctatctgattttgtgcaaaagcttcaaattaaaacagctgcattagaaaaagaggcgcttctcccctcccctacacctaaaggtgtatttaaactatcttgtgtgattaacttatttagagatgctgtaacttaaaataggggatatttaaggtagcttcagctagcttttaggaaaatcactttgtctaactcagaattatttttaaaaagaaatctggtcttgttagaaaacaaaattttattttgtgctcatttaagtttcaaacttactattttgacagttattttgataacaatgacactagaaaacttgactccatttcatcattgtttctgcatgaatatcatacaaatcagttagtttttaggtcaagggcttactatttctgggtcttttgctactaagttcacattagaattagtgccagaattttaggaacttcagagatcgtgtattgagatttcttaaataatgcttcagatattattgctttattgcttttttgtattggttaaaactgtacatttaaaattgctatgttactattttctacaattaatagtttgtctattttaaaataaattagttgttaagagtcttaa

SEQ ID NO: 9 - DNA, 5306 bp, Homo sapiens
ctaggcggcggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggcttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccacacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgac

SEQ ID NO: 10 - DNA, 4557 bp, Homo sapiens
cccctccattgggaccggagaaaccaggggagccccccgggcagccgcgcgccccttcccacggggccctttactgcgccgcgcgcccggcccccacccctcgcagcaccccgcgccccgcgccctcccagccgggtccagccggagccatggggccggagccgcagtgagcaccatggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcatctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaaggcccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagatgaaataaagacccagggggagaatgggtgttgtatggggaggcaagtgtggggggtccttctccacacccactttgtccatttgcaaatatattttggaaaaca

SEQ ID NO: 11 - DNA, 6459 bp, Homo sapiens
cttcccccaatcccctcaggctcggctgcgcccggggccgcgggccggtacctgaggtggcccaggcgccctccgcccgcggcgccgcccgggccgctcctccccgcgccccccgcgccccccgctcctccgcctccgcctccgcctccgcctcccccagctctccgcctcccttccccctccccgcccgacagcggccgctcgggccccggctctcggttataagatggcggcgctgagcggtggcggtggtggcggcgcggagccgggccaggctctgttcaacggggacatggagcccgaggccggcgccggcgccggcgccgcggcctcttcggctgcggaccctgccattccggaggaggtgtggaatatcaaacaaatgattaagttgacacaggaacatatagaggccctattggacaaatttggtggggagcataatccaccatcaatatatctggaggcctatgaagaatacaccagcaagctagatgcactccaacaaagagaacaacagttattggaatctctggggaacggaactgatttttctgtttctagctctgcatcaatggataccgttacatcttcttcctcttctagcctttcagtgctaccttcatctctttcagtttttcaaaatcccacagatgtggcacggagcaaccccaagtcaccacaaaaacctatcgttagagtcttcctgcccaacaaacagaggacagtggtacctgcaaggtgtggagttacagtccgagacagtctaaagaaagcactgatgatgagaggtctaatcccagagtgctgtgctgtttacagaattcaggatggagagaagaaaccaattggttgggacactgatatttcctggcttactggagaagaattgcatgtggaagtgttggagaatgttccacttacaacacacaactttgtacgaaaaacgtttttcaccttagcattttgtgacttttgtcgaaagctgcttttccagggtttccgctgtcaaacatgtggttataaatttcaccagcgttgtagtacagaagttccactgatgtgtgttaattatgaccaacttgatttgctgtttgtctccaagttctttgaacaccacccaataccacaggaagaggcgtccttagcagagactgccctaacatctggatcatccccttccgcacccgcctcggactctattgggccccaaattctcaccagtccgtctccttcaaaatccattccaattccacagcccttccgaccagcagatgaagatcatcgaaatcaatttgggcaacgagaccgatcctcatcagctcccaatgtgcatataaacacaatagaacctgtcaatattgatgacttgattagagaccaaggatttcgtggtgatggaggatcaaccacaggtttgtctgctaccccccctgcctcattacctggctcactaactaacgtgaaagccttacagaaatctccaggacctcagcgagaaaggaagtcatcttcatcctcagaagacaggaatcgaatgaaaacacttggtagacgggactcgagtgatgattgggagattcctgatgggcagattacagtgggacaaagaattggatctggatcatttggaacagtctacaagggaaagtggcatggtgatgtggcagtgaaaatgttgaatgtgacagcacctacacctcagcagttacaagccttcaaaaatgaagtaggagtactcaggaaaacacgacatgtgaatatcctactcttcatgggctattccacaaagccacaactggctattgttacccagtggtgtgagggctccagcttgtatcaccatctccatatcattgagaccaaatttgagatgatcaaacttatagatattgcacgacagactgcacagggcatggattacttacacgccaagtcaatcatccacagagacctcaagagtaataatatatttcttcatgaagacctcacagtaaaaataggtgattttggtctagctacagtgaaatctcgatggagtgggtcccatcagtttgaacagttgtctggatccattttgtggatggcaccagaagtcatcagaatgcaagataaaaatccatacagctttcagtcagatgtatatgcatttggaattgttctgtatgaattgatgactggacagttaccttattcaaacatcaacaacagggaccagataatttttatggtgggacgaggatacctgtctccagatctcagtaaggtacggagtaactgtccaaaagccatgaagagattaatggcagagtgcctcaaaaagaaaagagatgagagaccactctttccccaaattctcgcctctattgagctgctggcccgctcattgccaaaaattcaccgcagtgcatcagaaccctccttgaatcgggctggtttccaaacagaggattttagtctatatgcttgtgcttctccaaaaacacccatccaggcagggggatatggtgcgtttcctgtccactgaaacaaatgagtgagagagttcaggagagtagcaacaaaaggaaaataaatgaacatatgtttgcttatatgttaaattgaataaaatactctctttttttttaaggtgaaccaaagaacacttgtgtggttaaagactagatataatttttccccaaactaaaatttatacttaacattggatttttaacatccaagggttaaaatacatagacattgctaaaaattggcagagcctcttctagaggctttactttctgttccgggtttgtatcattcacttggttattttaagtagtaaacttcagtttctcatgcaacttttgttgccagctatcacatgtccactagggactccagaagaagaccctacctatgcctgtgtttgcaggtgagaagttggcagtcggttagcctgggttagataaggcaaactgaacagatctaatttaggaagtcagtagaatttaataattctattattattcttaataatttttctataactatttctttttataacaatttggaaaatgtggatgtcttttatttccttgaagcaataaactaagtttctttttataaattttgagtgcaggtgaccaaaaatattgctgaggagtggcacgtttgacatgagtaaaatgtcttaacttcggatttttagcgggaaaatgttataaattggagtttcttttaaatagctttttttaaaatacattaaggatgtctcgctcatgtagaagtcaaattttgttgcaaacgcattgctcccttcacacccaatctctcccctgcaaaaaatcttcacagaattctgtgagaacttttaggtgtgtttttctttgagatacctctggttgccaaacaccaggtaatagattttttaaagttgttattagattattcttacctctcatgatgcatattttagcaatcaccttatcattgtgtctcatgttctgtcctccttatattctttgcccagcaagattctacttatgatgaatgaatgctcttctccttttttcattcaatggtatgaagtatttgttagggttctttagtacttacactttgttgtgtagaaaatgactgtaatgtggtggtcagtgtattcttactgtgattcagagggaatcaaaagtagaaagcaacagcacgtggtcctatcaaagatttggccatctctgcttcactgtcagcctcttaactatatcttcacttactcaatttggttttgtcatgatttttaaatgtagccaatagatcaaggttcttccagtaaacacatatctgcataaatgcctccttgaagtcaataaagaaggaaattgagaagactttaaattaatgataatttagtttttaagtacccacaaataaatttttgaaacattttctttatttgaatacttagatgtcatccaggaaaatcactcaataataattacggcaaatctttaacccctcatttgggtagcttaagataagtaatgccattatgaatcagaattgattcatgactttagttaagaaaatgaaaaggaacatttcacgtatttttaaaaatgatactaaggaataaagaagtacaactattggaaaatatctaagtatatgatttttaaatcctccagtggcattaaatatatgattattagtaattgttagatagggttttattcattcacaaatagaagactagcaagcatgtaactaacaaagtttttacaaaattgactttgtggaatgctccaaatgtttggccattttgaggcacaaggtcaggggtctctttattgatagagctccttctataatttcccagcatacctgcctcacagttatcttcctttcattgttcactctctttttcttctcaatgccatcctgcctaggctcccatcatctgcatctgacacctttcctttctttctttactagtctcctttgcgatgggtgtggctaagctctgtagagccactcagaaactcattgttccattctgtagccagtaaaacatgcctccaaagtgtcacagagtaattctactctctcttttaaattaggtccaccggaaatgttagtgaaaggacattaaaaatgtgacaggtgacatgtttagctaacatggatctggagaaataggaagcagtagaattaaatgtttccctttcaggtttaattgtatttgttcttgggttttgttttatactgagttttaaatatattctccaaataaaaacattattttttctaaccatatgtagagttaatctctttgactaagtaattgaaacaaaagaacatttgttcttttgtgactgcttttttcctaaaacctgagccctcttttttttttttgaaattaaagttgatttcctttttttttttttttttttttttttttttttttttttttttgagacagagtctcgctctgtcgcccaggctggagtgcagtggcgggatctcggctcactgcaagctccgcctcccgggttcacgccattctcctgcctcagcctcccaagtagctgggactacaggcgcccgccactacgcccggctaattttttgtatttttagtagagacggggtttcaccgttttagccgggatggtctcgatctcctgacctcgtgatccgcccgcctcggcctcccaaagtgctgggattacaggcgtgagccaccgcgcccggccttaaaagttgatttccttcttcagtaaggaaacctttttataaatttgttttgcattttaaaagttttactaatcaatgatgaggaaaaagatttgtcttcttgattttaaatagtttcaggatcacaggatgtaatcagatgcttccagtttatttattttcaggtattacactagccatttaatcttttttatttatttattttcttcctgcccctcggatggcatataccagccatttagatactaaactctaatagttaaaccaatagttaaaattgtcctctctaaaacattggctatttaatataccagcttaaatggcctttctctcaagtgagtcactcttagtttaagaaaattatgtgcctttttaaaaaatattatgaaatggtacttcatgacagaaacattttatcagttatagtcttatttgattgaaaattgttgagcatttctgtaaaactttttactttactaaatatttcatctttcctgtgactgttttctcaaagaatttaaaagactcgatgtgtctatgccagaatgtttctcatccttttgaaactgcctgggccaggcgtagtggctcacgctgtaatcccagcacttttggaggccaaggtgggcagatcgcgtgagcccaggagtttgagaccagcctggacaacatggcgaaacggtgtctctacagaaaaatttaaaaattagccaagcatagtggtgcacaactgtagccccagccactcgggaggctgacgtgggaggatcccttgaacctgggggcggaggctgctgtgagccttcatcatgccactgcactccagcctgggcaacaaagcaaaaccctgtctcaaaaaaagaaaagaaaaaaagaaactgcttgaaagtcatgacgaagaatgtcaggaggggacttattctggctgcagttgactttctccttaaatgtcaagtagtgattgatttggataagaagtaaactgttacttttcataacatactttaaggaatttatcaaattctatgtataatgcccattaaaatatactccattctggagtaaagggtaagagtaatatttttaaactagttaataaagtctttagctttcacataaaccatgatatttgaggtgtctaaaatcacagggtcttttttttttttttcagtcttcccagttgttctctgctctattcctaaataaagttaacttgaaaatgca

SEQ ID NO: 12 - DNA, 8595 bp, Homo sapiens
ctcctttctccggcagcagaaagcggagagtcacagcggggccaggccctggggagcggagcctccaccgcccccctcattcccaggcaagggcttggggggaatgagccgggagagccgggtcccgagcctacagagccgggagcagctgagccgccggcgcctcggccgccgccgccgcctcctcctcctccgccgccgccagcccggagcctgagccggcggggcgggggggagaggagcgagcgcagcgcagcagcggagccccgcgaggcccgcccgggcgggtggggagggcagcccgggggactgggccccggggcggggtgggagggggggagaagacgaagacagggccgggtctctccgcggacgagacagcggggatcatggccgcgcaggtcgcccccgccgccgccagcagcctgggcaacccgccgccgccgccgccctcggagctgaagaaagccgagcagcagcagcgggaggaggcggggggcgaggcggcggcggcggcagcggccgagcgcggggaaatgaaggcagccgccgggcaggaaagcgagggccccgccgtggggccgccgcagccgctgggaaaggagctgcaggacggggccgagagcaatgggggtggcggcggcggcggagccggcagcggcggcgggcccggcgcggagccggacctgaagaactcgaacgggaacgcgggccctaggcccgccctgaacaataacctcacggagccgcccggcggcggcggtggcggcagcagcgatggggtgggggcgcctcctcactcagccgcggccgccttgccgcccccagcctacggcttcgggcaaccctacggccggagcccgtctgccgtcgccgccgccgcggccgccgtcttccaccaacaacatggcggacaacaaagccctggcctggcagcgctgcagagcggcggcggcgggggcctggagccctacgcggggccccagcagaactctcacgaccacggcttccccaaccaccagtacaactcctactaccccaaccgcagcgcctaccccccgcccgccccggcctacgcgctgagctccccgagaggtggcactccgggctccggcgcggcggcggctgccggctccaagccgcctccctcctccagcgcctccgcctcctcgtcgtcttcgtccttcgctcagcagcgcttcggggccatggggggaggcggcccctccgcggccggcgggggaactccccagcccaccgccacccccaccctcaaccaactgctcacgtcgcccagctcggcccggggctaccagggctaccccgggggcgactacagtggcgggccccaggacgggggcgccggcaagggcccggcggacatggcctcgcagtgttggggggctgcggcggcggcagctgcggcggcggccgcctcgggaggggcccaacaaaggagccaccacgcgcccatgagccccgggagcagcggcggcggggggcagccgctcgcccggacccctcagccatccagtccaatggatcagatgggcaagatgagacctcagccatatggcgggactaacccatactcgcagcaacagggacctccgtcaggaccgcagcaaggacatgggtacccagggcagccatacgggtcccagaccccgcagcggtacccgatgaccatgcagggccgggcgcagagtgccatgggcggcctctcttatacacagcagattcctccttatggacaacaaggccccagcgggtatggtcaacagggccagactccatattacaaccagcaaagtcctcaccctcagcagcagcagccaccctactcccagcaaccaccgtcccagacccctcatgcccaaccttcgtatcagcagcagccacagtctcaaccaccacagctccagtcctctcagcctccatactcccagcagccatcccagcctccacatcagcagtccccggctccatacccctcccagcagtcgacgacacagcagcacccccagagccagcccccctactcacagccacaggctcagtctccttaccagcagcagcaacctcagcagccagcaccctcgacgctctcccagcaggctgcgtatcctcagccccagtctcagcagtcccagcaaactgcctattcccagcagcgcttccctccaccgcaggagctatctcaagattcatttgggtctcaggcatcctcagccccctcaatgacctccagtaagggagggcaagaagatatgaacctgagccttcagtcaagaccctccagcttgcctgatctatctggttcaatagatgacctccccatggggacagaaggagctctgagtcctggagtgagcacatcagggatttccagcagccaaggagagcagagtaatccagctcagtctcctttctctcctcatacctcccctcacctgcctggcatccgaggcccttccccgtcccctgttggctctcccgccagtgttgctcagtctcgctcaggaccactctcgcctgctgcagtgccaggcaaccagatgccacctcggccacccagtggccagtcggacagcatcatgcatccttccatgaaccaatcaagcattgcccaagatcgaggttatatgcagaggaacccccagatgccccagtacagttccccccagcccggctcagccttatctccgcgtcagccttccggaggacagatacacacaggcatgggctcctaccagcagaactccatggggagctatggtccccaggggggtcagtatggcccacaaggtggctaccccaggcagccaaactataatgccttgcccaatgccaactaccccagtgcaggcatggctggaggcataaaccccatgggtgccggaggtcaaatgcatggacagcctggcatcccaccttatggcacactccctccagggaggatgagtcacgcctccatgggcaaccggccttatggccctaacatggccaatatgccacctcaggttgggtcagggatgtgtcccccaccagggggcatgaaccggaaaacccaagaaactgctgtcgccatgcatgttgctgccaactctatccaaaacaggccgccaggctaccccaatatgaatcaagggggcatgatgggaactggacctccttatggacaagggattaatagtatggctggcatgatcaaccctcagggacccccatattccatgggtggaaccatggccaacaattctgcagggatggcagccagcccagagatgatgggccttggggatgtaaagttaactccagccaccaaaatgaacaacaaggcagatgggacacccaagacagaatccaaatccaagaaatccagttcttctactacaaccaatgagaagatcaccaagttgtatgagctgggtggtgagcctgagaggaagatgtgggtggaccgttatctggccttcactgaggagaaggccatgggcatgacaaatctgcctgctgtgggtaggaaacctctggacctctatcgcctctatgtgtctgtgaaggagattggtggattgactcaggtcaacaagaacaaaaaatggcgggaacttgcaaccaacctcaatgtgggcacatcaagcagtgctgccagctccttgaaaaagcagtatatccagtgtctctatgcctttgaatgcaagattgaacggggagaagaccctcccccagacatctttgcagctgctgattccaagaagtcccagcccaagatccagcctccctctcctgcgggatcaggatctatgcaggggccccagactccccagtcaaccagcagttccatggcagaaggaggagacttaaagccaccaactccagcatccacaccacacagtcagatccccccattgccaggcatgagcaggagcaattcagttgggatccaggatgcctttaatgatggaagtgactccacattccagaagcggaattccatgactccaaaccctgggtatcagcccagtatgaatacctctgacatgatggggcgcatgtcctatgagccaaataaggatccttatggcagcatgaggaaagctccagggagtgatcccttcatgtcctcagggcagggccccaacggcgggatgggtgacccctacagtcgtgctgccggccctgggctaggaaatgtggcgatgggaccacgacagcactatccctatggaggtccttatgacagagtgaggacggagcctggaatagggcctgagggaaacatgagcactggggccccacagccgaatctcatgccttccaacccagactcggggatgtattctcctagccgctaccccccgcagcagcagcagcagcagcagcaacgacatgattcctatggcaatcagttctccacccaaggcaccccttctggcagccccttccccagccagcagactacaatgtatcaacagcaacagcagaattacaagcggccaatggatggcacatatggccctcctgccaagcggcacgaaggggagatgtacagcgtgccatacagcactgggcaggggcagcctcagcagcagcagttgcccccagcccagccccagcctgccagccagcaacaagctgcccagccttcccctcagcaagatgtatacaaccagtatggcaatgcctatcctgccactgccacagctgctactgagcgccgaccagcaggcggcccccagaaccaatttccattccagtttggccgagaccgtgtctctgcaccccctggcaccaatgcccagcaaaacatgccaccacaaatgatgggcggccccatacaggcatcagctgaggttgctcagcaaggcaccatgtggcaggggcgtaatgacatgacctataattatgccaacaggcagagcacgggctctgccccccagggccccgcctatcatggcgtgaaccgaacagatgaaatgctgcacacagatcagagggccaaccacgaaggctcgtggccttcccatggcacacgccagcccccatatggtccctctgcccctgtgccccccatgacaaggccccctccatctaactaccagcccccaccaagcatgcagaatcacattcctcaggtatccagccctgctcccctgccccggccaatggagaaccgcacctctcctagcaagtctccattcctgcactctgggatgaaaatgcagaaggcaggtcccccagtacctgcctcgcacatagcacctgcccctgtgcagccccccatgattcggcgggatatcaccttcccacctggctctgttgaagccacacagcctgtgttgaagcagaggaggcggctcacaatgaaagacattggaaccccggaggcatggcgggtaatgatgtccctcaagtctggtctcctggcagagagcacatgggcattagataccatcaacatcctgctgtatgatgacaacagcatcatgaccttcaacctcagtcagctcccagggttgctagagctccttgtagaatatttccgacgatgcctgattgagatctttggcattttaaaggagtatgaggtgggtgacccaggacagagaacgctactggatcctgggaggttcagcaaggtgtctagtccagctcccatggagggtggggaagaagaagaagaacttctaggtcctaaactagaagaggaagaagaagaggaagtagttgaaaatgatgaggagatagccttttcaggcaaggacaagccagcttcagagaatagtgaggagaagctgatcagtaagtttgacaagcttccagtaaagatcgtacagaagaatgatccatttgtggtggactgctcagataagcttgggcgtgtgcaggagtttgacagtggcctgctgcactggcggattggtgggggggacaccactgagcatatccagacccacttcgagagcaagacagagctgctgccttcccggcctcacgcaccctgcccaccagcccctcggaagcatgtgacaacagcagagggtacaccagggacaacagaccaggaggggcccccacctgatggacctccagaaaaacggatcacagccactatggatgacatgttgtctactcggtctagcaccttgaccgaggatggagctaagagttcagaggccatcaaggagagcagcaagtttccatttggcattagcccagcacagagccaccggaacatcaagatcctagaggacgaaccccacagtaaggatgagaccccactgtgtacccttctggactggcaggattctcttgccaagcgctgcgtctgtgtgtccaataccattcgaagcctgtcatttgtgccaggcaatgactttgagatgtccaaacacccagggctgctgctcatcctgggcaagctgatcctgctgcaccacaagcacccagaacggaagcaggcaccactaacttatgaaaaggaggaggaacaggaccaaggggtgagctgcaacaaagtggagtggtggtgggactgcttggagatgctccgggaaaacaccttggttacactcgccaacatctcggggcagttggacctatctccataccccgagagcatttgcctgcctgtcctggacggactcctacactgggcagtttgcccttcagctgaagcccaggaccccttttccaccctgggccccaatgccgtcctttccccgcagagactggtcttggaaaccctcagcaaactcagcatccaggacaacaatgtggacctgattctggccacaccccccttcagccgcctggagaagttgtatagcactatggtgcgcttcctcagtgaccgaaagaacccggtgtgccgggagatggctgtggtactgctggccaacctggctcagggggacagcctggcagctcgtgccattgcagtgcagaagggcagtatcggcaacctcctgggcttcctagaggacagccttgccgccacacagttccagcagagccaggccagcctcctccacatgcagaacccaccctttgagccaactagtgtggacatgatgcggcgggctgcccgcgcgctgcttgccttggccaaggtggacgagaaccactcagagtttactctgtacgaatcacggctgttggacatctcggtatcaccgttgatgaactcattggtttcacaagtcatttgtgatgtactgtttttgattggccagtcatgacagccgtgggacacctcccccccccgtgtgtgtgtgcgtgtgtggagaacttagaaactgactgttgccctttatttatgcaaaaccacctcagaatccagtttaccctgtgctgtccagcttctcccttgggaaaaagtctctcctgtttctctctcctccttccacctcccctccctccatcacctcacgcctttctgttccttgtcctcaccttactcccctcaggaccctaccccaccctctttgaaaagacaaagctctgcctacatagaagactttttttattttaaccaaagttactgttgtttacagtgagtttggggaaaaaaaataaaataaaaatggctttcccagtccttgcatcaacgggatgccacatttcataactgtttttaatggtaaaaaaaaaaaaaaaaaatacaaaaaaaaattctgaaggacaaaaaaggtgactgctgaactgtgtgtggtttattgttgtacattcacaatcttgcaggagccaagaagttcgcagttgtgaacagaccctgttcactggagaggcctgtgcagtagagtgtagaccctttcatgtactgtactgtacacctgatactgtaaacatactgtaataataatgtctcacatggaaacagaaaacgctgggtcagcagcaagctgtagtttttaaaaatgtttttagttaaacgttgaggagaaaaaaaaaaaaggcttttcccccaaagtatcatgtgtgaacctacaacaccctgacctctttctctcctccttgattgtatgaataaccctgagatcacctcttagaactggttttaacctttagctgcagcggctacgctgccacgtgtgtatatatatgacgttgtacattgcacatacccttggatccccacagtttggtcctcctcccagctacccctttatagtatgacgagttaacaagttggtgacctgcacaaagcgagacacagctatttaatctcttgccagatatcgcccctcttggtgcgatgctgtacaggtctctgtaaaaagtccttgctgtctcagcagccaatcaacttatagtttatttttttctgggtttttgttttgttttgttttctttctaatcgaggtgtgaaaaagttctaggttcagttgaagttctgatgaagaaacacaattgagattttttcagtgataaaatctgcatatttgtatttcaacaatgtagctaaaacttgatgtaaattcctcctttttttccttttttggcttaatgaatatcatttattcagtatgaaatctttatactatatgttccacgtgttaagaataaatgtacattaaatcttggtaa

SEQ ID NO: 13 - DNA, 8515 bp, Homo sapiens
gttctctcctctcggaagctgcagccatgatggaagtttgagagttgagccgctgtgaggcgaggccgggctcaggcgagggagatgagagacggcggcggccgcggcccggagcccctctcagcgcctgtgagcagccgcgggggcagcgccctcggggagccggccggcctgcggcggcggcagcggcggcgtttctcgcctcctcttcgtcttttctaaccgtgcagcctcttcctcggcttctcctgaaagggaaggtggaagccgtgggctcgggcgggagccggctgaggcgcggcggcggcggcggcacctcccgctcctggagcgggggggagaagcggcggcggcggcggccgcggcggctgcagctccagggagggggtctgagtcgcctgtcaccatttccagggctgggaacgccggagagttggtctctccccttctactgcctccaacacggcggcggcggcggctggcacatccagggacccgggccggttttaaacctcccgtgcgccgccgccgcaccccccgtggcccgggctccggaggccgccggcggaggcagccgttcggaggattattcgtcttctccccattccgctgccgccgctgccaggcctctggctgctgaggagaagcaggcccagtcgctgcaaccatccagcagccgccgcagcagccattacccggctgcggtccagagccaagcggcggcagagcgaggggcatcagctaccgccaagtccagagccatttccatcctgcagaagaagccccgccaccagcagcttctgccatctctctcctcctttttcttcagccacaggctcccagacatgacagccatcatcaaagagatcgttagcagaaacaaaaggagatatcaagaggatggattcgacttagacttgacctatatttatccaaacattattgctatgggatttcctgcagaaagacttgaaggcgtatacaggaacaatattgatgatgtagtaaggtttttggattcaaagcataaaaaccattacaagatatacaatctttgtgctgaaagacattatgacaccgccaaatttaattgcagagttgcacaatatccttttgaagaccataacccaccacagctagaacttatcaaacccttttgtgaagatcttgaccaatggctaagtgaagatgacaatcatgttgcagcaattcactgtaaagctggaaagggacgaactggtgtaatgatatgtgcatatttattacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatatattcctccaattcaggacccacacgacgggaagacaagttcatgtactttgagttccctcagccgttacctgtgtgtggtgatatcaaagtagagttcttccacaaacagaacaagatgctaaaaaaggacaaaatgtttcacttttgggtaaatacattcttcataccaggaccagaggaaacctcagaaaaagtagaaaatggaagtctatgtgatcaagaaatcgatagcatttgcagtatagagcgtgcagataatgacaaggaatatctagtacttactttaacaaaaaatgatcttgacaaagcaaataaagacaaagccaaccgatacttttctccaaattttaaggtgaagctgtacttcacaaaaacagtagaggagccgtcaaatccagaggctagcagttcaacttctgtaacaccagatgttagtgacaatgaacctgatcattatagatattctgacaccactgactctgatccagagaatgaaccttttgatgaagatcagcatacacaaattacaaaagtctgaatttttttttatcaagagggataaaacaccatgaaaataaacttgaataaactgaaaatggacctttttttttttaatggcaataggacattgtgtcagattaccagttataggaacaattctcttttcctgaccaatcttgttttaccctatacatccacagggttttgacacttgttgtccagttgaaaaaaggttgtgtagctgtgtcatgtatatacctttttgtgtcaaaaggacatttaaaattcaattaggattaataaagatggcactttcccgttttattccagttttataaaaagtggagacagactgatgtgtatacgtaggaattttttccttttgtgttctgtcaccaactgaagtggctaaagagctttgtgatatactggttcacatcctacccctttgcacttgtggcaacagataagtttgcagttggctaagagaggtttccgaagggttttgctacattctaatgcatgtattcgggttaggggaatggagggaatgctcagaaaggaaataattttatgctggactctggaccatataccatctccagctatttacacacacctttctttagcatgctacagttattaatctggacattcgaggaattggccgctgtcactgcttgttgtttgcgcatttttttttaaagcatattggtgctagaaaaggcagctaaaggaagtgaatctgtattggggtacaggaatgaaccttctgcaacatcttaagatccacaaatgaagggatataaaaataatgtcataggtaagaaacacagcaacaatgacttaaccatataaatgtggaggctatcaacaaagaatgggcttgaaacattataaaaattgacaatgatttattaaatatgttttctcaattgtaacgacttctccatctcctgtgtaatcaaggccagtgctaaaattcagatgctgttagtacctacatcagtcaacaacttacacttattttactagttttcaatcataatacctgctgtggatgcttcatgtgctgcctgcaagcttcttttttctcattaaatataaaatattttgtaatgctgcacagaaattttcaatttgagattctacagtaagcgttttttttctttgaagatttatgatgcacttattcaatagctgtcagccgttccacccttttgaccttacacattctattacaatgaattttgcagttttgcacattttttaaatgtcattaactgttagggaattttacttgaatactgaatacatataatgtttatattaaaaaggacatttgtgttaaaaaggaaattagagttgcagtaaactttcaatgctgcacacaaaaaaaagacatttgatttttcagtagaaattgtcctacatgtgctttattgatttgctattgaaagaatagggtttttttttttttttttttttttttttttaaatgtgcagtgttgaatcatttcttcatagtgctcccccgagttgggactagggcttcaatttcacttcttaaaaaaaatcatcatatatttgatatgcccagactgcatacgattttaagcggagtacaactactattgtaaagctaatgtgaagatattattaaaaaggtttttttttccagaaatttggtgtcttcaaattataccttcaccttgacatttgaatatccagccattttgtttcttaatggtataaaattccattttcaataacttattggtgctgaaattgttcactagctgtggtctgacctagttaatttacaaatacagattgaataggacctactagagcagcatttatagagtttgatggcaaatagattaggcagaacttcatctaaaatattcttagtaaataatgttgacacgttttccataccttgtcagtttcattcaacaatttttaaatttttaacaaagctcttaggatttacacatttatatttaaacattgatatatagagtattgattgattgctcataagttaaattggtaaagttagagacaactattctaacacctcaccattgaaatttatatgccaccttgtctttcataaaagctgaaaattgttacctaaaatgaaaatcaacttcatgttttgaagatagttataaatattgttctttgttacaatttcgggcaccgcatattaaaacgtaactttattgttccaatatgtaacatggagggccaggtcataaataatgacattataatgggcttttgcactgttattatttttcctttggaatgtgaaggtctgaatgagggttttgattttgaatgtttcaatgtttttgagaagccttgcttacattttatggtgtagtcattggaaatggaaaaatggcattatatatattatatatataaatatatattatacatactctccttactttatttcagttaccatccccatagaatttgacaagaattgctatgactgaaaggttttcgagtcctaattaaaactttatttatggcagtattcataattagcctgaaatgcattctgtaggtaatctctgagtttctggaatattttcttagactttttggatgtgcagcagcttacatgtctgaagttacttgaaggcatcacttttaagaaagcttacagttgggccctgtaccatcccaagtcctttgtagctcctcttgaacatgtttgccatacttttaaaagggtagttgaataaatagcatcaccattctttgctgtggcacaggttataaacttaagtggagtttaccggcagcatcaaatgtttcagctttaaaaaataaaagtagggtacaagtttaatgtttagttctagaaattttgtgcaatatgttcataacgatggctgtggttgccacaaagtgcctcgtttacctttaaatactgttaatgtgtcatgcatgcagatggaaggggtggaactgtgcactaaagtgggggctttaactgtagtatttggcagagttgccttctacctgccagttcaaaagttcaacctgttttcatatagaatatatatactaaaaaatttcagtctgttaaacagccttactctgattcagcctcttcagatactcttgtgctgtgcagcagtggctctgtgtgtaaatgctatgcactgaggatacacaaaaataccaatatgatgtgtacaggataatgcctcatcccaatcagatgtccatttgttattgtgtttgttaacaaccctttatctcttagtgttataaactccacttaaaactgattaaagtctcattcttgtcattgtgtgggtgttttattaaatgagagtttataattcaaattgcttaagtccattgaagttttaattaatgggcagccaaatgtgaatacaaagttttcagtttttttttttcctgctgtccttcaaagcctactgtttaaaaaaaaaaaaaaaaaaaaacatggcctgagagtagagtatctgtctactcatgtttaattaaggaaaaacacttatttttagggctttagtcatcacttcataaattgtataagcacattaaatagcgttctagtcctgaaaaagtccaagattcttagaaaattgtgcatatttttattatgacagatgtttgaagataattccccagaatggatttgatactttagatttcaattttgtggcttttgtctattattctgtactctgccatcagcatatggaaagcttcatttactcatcatgacttgtgccatataaaaattgatatttcggaatagtctaaaggactttttgtacttgaatttaatcatgttgtttctaatattcttaaaagcttgaagactaaagcatatcctttcaacaaagcatagtaaggtaataagaaagtgtagtttgtacaagtgttaaaaaaataaagtagacaatgttacagtgggacttattatttcaagtttacattttctccatgtaattttttaaaaagtaaatgaaaaaatgtgcaataatgtaaaatatgaagtgtatgtgtacacacattttatttttcggtatcttgggtatacgtatggttgaaaactatactggagtctaaaagtattctaatttataagaagacattttggtgatgtttgaaaaatagaaatgtgctagttttgtttttatatcatgtcctttgtacgttgtaatatgagctggcttggttcagtaaatgccatcaccatttccattgagaatttaaaactcaccagtgtttaatatgcaggcttccaaaggcttatgaaaaaaatcaagacccttaaatctagttaatttgctgctaacatgaaactctttggttcttttatttttgccagataattagacacacatctaaagcttagtcttaaatggcttaagtgtagctattgattagtgctgttgctagttcagaaagaaatgtttgtgaatggaaacaagaatattcagtccaaactgttgtaaggacagtacctgaaaaccaggaaacaggataatggaaaaagtcttttaaagatgaaatgttggagccaactttcttatagaattaattgtatgtggctatagaaagcctaatgattgttgcttatttttgagagcatattattcttttatgaccataatcttgctgtttttccatcttccaaaagatcttccttctaatatgtatatcagaatgtgggtagccagtcagacaaattcatattggttggtagctttaaaaagtttgtaatgtgaagacaggaaaggacaaaatagtttgctttggtggtagtactctggttgttaagctaggtattttgagactacttccccatcacaacaacaataaaataatcactcataatcctatcacctggagacatagccatcgttaatatgttagtgactatacaatcatgttttcttctgtatatccatgtatattctttaaaaatgaaatttatactgtacctgatctcaaagctttttagcttagtatatctgtcatgaatttgtaggatgttccattgcatcagaaaacggacagtgatttgattactttctaatgccacagatgcagattacatgtagttattgagaatcctttcgaattcagtggcttaatcatgaatgtctaaatattgttgacattaggatgatacatgtaaattaaagttacatttgtttagcatagacaagcttaacattgtagatgtttctcttcaaaaatcatcttaaacatttgcatttggaattgtgttaaatagaatgtgtgaaacactgtattagtaaacttcatcacctttctacttccttatagtttgaacttttcagtttttgtagttcccaaacagttgctcaatttagagcaaattaatttaacacctgccaaaaaaaggctgctgttggcttatcagttgtctttaaattcaaatgctcatgtgacttttatcacatcaaaaaatatttcattaatgattcacctttagctctgaaaattaccgcgtttagtaattatagtgggcttataaaaacatgcaactctttttgatagttatttgagaattttggtgaaaaatatttagctgagggcagtatagaacttataaaccaatatattgatatttttaaaacatttttacatataagtaaactgccatctttgagcataactacatttaaaaataaagctgcatatttttaaatcaagtgtttaacaagaatttatattttttattttttaaaattaaaaataatttatatttcctctgttgcatgaggattctcatctgtgcttataatggttagagattttatttgtgtggaatgaagtgaggcttgtagtcatggttctagtgtttcagtttgccaagtctgtttactgcagtgaaattcatcaaatgtttcagtgtggttttctgtagcctatcatttactggctatttttttatgtacacctttaggattttctgcctactctatccagttgtccaaatgatatcctacattttacaaatgccctttcagtttctattttctttttccattaaattgccctcatgtcctaatgtgcagtttgtaagtgtgtgtgtgtgtgtctgtgtgtgtgtgaatttgattttcaagagtgctagacttccaatttgagagattaaataatttaattcaggcaaacatttttcattggaatttcacagttcattgtaatgaaaatgttaatcctggatgacctttgacatacagtaatgaatcttggatattaatgaatttgttagtagcatcttgatgtgtgttttaatgagttattttcaaagttgtgcattaaaccaaagttggcatactggaagtgtttatatcaagttccatttggctactgatggacaaaaaatagaaatgccttcctatggagagtatttttcctttaaaaaattaaaaaggttaattattttgacta

SEQ ID NO: 14 - DNA, 4624 bp, Homo sapiens
gagagcgcggtggagagccgagcgggcgggcggcgggtgcggagcgggcgagggagcgcgcgcggccgccacaaagctcgggcgccgcggggctgcatgcggcgtacctggcccggcgcggcgactgctctccgggctggcgggggccggccgcgagccccgggggccccgaggccgcagcttgcctgcgcgctctgagccttcgcaactcgcgagcaaagtttggtggaggcaacgccaagcctgagtcctttcttcctctcgttccccaaatccgagggcagcccgcgggcgtcatgcccgcgctcctccgcagcctggggtacgcgtgaagcccgggaggcttggcgccggcgaagacccaaggaccactcttctgcgtttggagttgctccccgcaaccccgggctcgtcgctttctccatcccgacccacgcggggcgcggggacaacacaggtcgcggaggagcgttgccattcaagtgactgcagcagcagcggcagcgcctcggttcctgagcccaccgcaggctgaaggcattgcgcgtagtccatgcccgtagaggaagtgtgcagatgggattaacgtccacatggagatatggaagaggaccggggattggtaccgtaaccatggtcagctggggtcgtttcatctgcctggtcgtggtcaccatggcaaccttgtccctggcccggccctccttcagtttagttgaggataccacattagagccagaagagccaccaaccaaataccaaatctctcaaccagaagtgtacgtggctgcgccaggggagtcgctagaggtgcgctgcctgttgaaagatgccgccgtgatcagttggactaaggatggggtgcacttggggcccaacaataggacagtgcttattggggagtacttgcagataaagggcgccacgcctagagactccggcctctatgcttgtactgccagtaggactgtagacagtgaaacttggtacttcatggtgaatgtcacagatgccatctcatccggagatgatgaggatgacaccgatggtgcggaagattttgtcagtgagaacagtaacaacaagagagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtagtggagaatgaatacgggtccatcaatcacacgtaccacctggatgttgtggagcgatcgcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctgccagcgcctggaagagaaaaggagattacagcttccccagactacctggagatagccatttactgcataggggtcttcttaatcgcctgtatggtggtaacagtcatcctgtgccgaatgaagaacacgaccaagaagccagacttcagcagccagccggctgtgcacaagctgaccaaacgtatccccctgcggagacaggtaacagtttcggctgagtccagctcctccatgaactccaacaccccgctggtgaggataacaacacgcctctcttcaacggcagacacccccatgctggcaggggtctccgagtatgaacttccagaggacccaaaatgggagtttccaagagataagctgacactgggcaagcccctgggagaaggttgctttgggcaagtggtcatggcggaagcagtgggaattgacaaagacaagcccaaggaggcggtcaccgtggccgtgaagatgttgaaagatgatgccacagagaaagacctttctgatctggtgtcagagatggagatgatgaagatgattgggaaacacaagaatatcataaatcttcttggagcctgcacacaggatgggcctctctatgtcatagttgagtatgcctctaaaggcaacctccgagaatacctccgagcccggaggccacccgggatggagtactcctatgacattaaccgtgttcctgaggagcagatgaccttcaaggacttggtgtcatgcacctaccagctggccagaggcatggagtacttggcttcccaaaaatgtattcatcgagatttagcagccagaaatgttttggtaacagaaaacaatgtgatgaaaatagcagactttggactcgccagagatatcaacaatatagactattacaaaaagaccaccaatgggcggcttccagtcaagtggatggctccagaagccctgtttgatagagtatacactcatcagagtgatgtctggtccttcggggtgttaatgtgggagatcttcactttagggggctcgccctacccagggattcccgtggaggaactttttaagctgctgaaggaaggacacagaatggataagccagccaactgcaccaacgaactgtacatgatgatgagggactgttggcatgcagtgccctcccagagaccaacgttcaagcagttggtagaagacttggatcgaattctcactctcacaaccaatgaggaatacttggacctcagccaacctctcgaacagtattcacctagttaccctgacacaagaagttcttgttcttcaggagatgattctgttttttctccagaccccatgccttacgaaccatgccttcctcagtatccacacataaacggcagtgttaaaacatgaatgactgtgtctgcctgtccccaaacaggacagcactgggaacctagctacactgagcagggagaccatgcctcccagagcttgttgtctccacttgtatatatggatcagaggagtaaataattggaaaagtaatcagcatatgtgtaaagatttatacagttgaaaacttgtaatcttccccaggaggagaagaaggtttctggagcagtggactgccacaagccaccatgtaacccctctcacctgccgtgcgtactggctgtggaccagtaggactcaaggtggacgtgcgttctgccttccttgttaattttgtaataattggagaagatttatgtcagcacacacttacagagcacaaatgcagtatataggtgctggatgtatgtaaatatattcaaattatgtataaatatatattatatatttacaaggagttattttttgtattgattttaaatggatgtcccaatgcacctagaaaattggtctctctttttttaatagctatttgctaaatgctgttcttacacataatttcttaattttcaccgagcagaggtggaaaaatacttttgctttcagggaaaatggtataacgttaatttattaataaattggtaatatacaaaacaattaatcatttatagttttttttgtaatttaagtggcatttctatgcaggcagcacagcagactagttaatctattgcttggacttaactagttatcagatcctttgaaaagagaatatttacaatatatgactaatttggggaaaatgaagttttgatttatttgtgtttaaatgctgctgtcagacgattgttcttagacctcctaaatgccccatattaaaagaactcattcataggaaggtgtttcattttggtgtgcaaccctgtcattacgtcaacgcaacgtctaactggacttcccaagataaatggtaccagcgtcctcttaaaagatgccttaatccattccttgaggacagaccttagttgaaatgatagcagaatgtgcttctctctggcagctggccttctgcttctgagttgcacattaatcagattagcctgtattctcttcagtgaattttgataatggcttccagactctttggcgttggagacgcctgttaggatcttcaagtcccatcatagaaaattgaaacacagagttgttctgctgatagttttggggatacgtccatctttttaagggattgctttcatctaattctggcaggacctcaccaaaagatccagcctcatacctacatcagacaaaatatcgccgttgttccttctgtactaaagtattgtgttttgctttggaaacacccactcactttgcaatagccgtgcaagatgaatgcagattacactgatcttatgtgttacaaaattggagaaagtatttaataaaacctgttaatttttatactgacaataaaaatgtttctacagatattaatgttaacaagacaaaataaatgtcacgcaacttatttttttaa

SEQ ID NO: 15 - DNA, 10705 bp, Homo sapiens
gagtcaagatggcggccaaggtggcgaagcagcagccgcggcggcggcggcggctggagtgagcgtccgactcgccgcgccgaacgaggtcccggtgtagggccgcgcgccgtggccgcgtcccactcctcaggccggggcgcacgtcggctcccacgcttagccagctcccggtggtttcctagaaacatgattgtttattggcattgatctcacagtctggtgaggacttctttactgataatgtcaagttcaggttatcctcccaaccaaggagcattcagcacagaacaaagtcgttatcctcctcactctgtccagtatacatttcccaacacccgccaccagcaggagttcgcagtccctgattatcgttcctctcatcttgaagtgagtcaggcatcacagcttttgcagcaacagcagcagcaacagcttcgaaggcgaccttccttgctttcagaatttcacccaggttctgacaggcctcaagaaaggagaactagttatgaaccgtttcatccaggcccatccccagtggatcatgattcactggaatcgaagcgaccacgtctggaacaggtttctgattctcattttcagcgtgtcagtgctgcggttttgcctttagtgcacccgctgccagaagggctgagggcttctgcagatgctaagaaggatccagcattcggaggcaaacatgaagctccatcctctccaatttcggggcaaccatgtggagatgatcaaaatgcttcaccttcaaaactctcaaaggaagagttaatacagagtatggatcgtgtagatcgagaaattgcaaaagtagaacagcagatccttaaactgaaaaagaaacaacaacagcttgaagaagaggcagctaaacctcctgagcctgagaagcccgtgtcccctcctcctgtggagcagaaacaccgcagtattgtccaaattatttatgatgagaatcggaaaaaagcagaagaagctcataaaatttttgaaggtcttggcccaaaagttgaactgccactgtataaccagccatcagataccaaggtgtaccatgagaacatcaagacaaaccaggtgatgaggaaaaaactcattttattttttaaaagaagaaatcatgcaagaaaacaaagggaacaaaaaatctgccagcgttatgatcagctcatggaggcatgggagaaaaaagtggacagaatagaaaataatcctcggaggaaagctaaagaaagcaaaacaagggaatactatgaaaagcagtttccagaaattcgaaaacaaagagaacagcaagaaagatttcagcgagttgggcagaggggagctggtctttcagccaccattgctaggagtgagcatgagatttctgaaattattgatgggctctctgagcaggagaataatgagaaacaaatgcggcagctctctgtgattccacctatgatgtttgatgcagaacaaagacgagtcaagttcattaacatgaatgggcttatggaggaccctatgaaagtgtataaagataggcagtttatgaatgtttggactgaccatgaaaaggagatctttaaggacaagtttatccagcatccaaaaaactttggactaattgcatcatacttggagaggaagagtgttcctgattgtgttttgtattactatttaaccaagaaaaatgagaattataaagccctcgtcagaaggaattatgggaaacgcagaggcagaaaccagcaaattgctcgaccctcgcaagaagaaaaagtagaagaaaaagaagaggataaagcagaaaaaacagaaaaaaaagaagaagaaaagaaagatgaagaggaaaaagatgaaaaagaagactccaaagaaaataccaaggaaaaggacaagatagatggtacagcagaagaaactgaggaaagagagcaagccacaccccgggggcgaaagactgccaacagtcagggccgccgtaagggccggatcaccaggtccatgacaaacgaagctgcagctgccagtgctgcagccgcagcggctactgaagagcccccaccacctctgccaccgccaccagaacccatttctacagagcctgtggagacctctcgatggacagaagaagaaatggaagttgctaaaaaaggtctagtagaacatggtcgtaactgggcagcaattgctaaaatggtgggaacgaaaagtgaagctcaatgtaaaaacttctattttaactataaaaggcgacacaatcttgacaacctcttacagcagcataaacagaaaacttcacgaaaacctcgtgaagagcgagatgtgtctcaatgtgaaagtgtcgcttccactgtttctgctcaggaggatgaagatattgaagcctccaatgaagaagaaaatccagaagacagcgaagttgaagctgtcaagcccagcgaggacagtcctgaaaatgctacttctcgaggaaacacagaacctgcggttgagcttgagcccaccacggaaactgcacccagtacatctccctccttagcagttccaagtacaaaaccagctgaagatgaaagtgtggagacccaggtgaatgacagcatcagtgctgagacagcagagcagatggatgtagatcagcaggagcacagtgctgaagagggttctgtttgtgatcccccacccgctaccaaagctgactctgtggacgttgaagtgagggtgccagaaaaccatgcatctaaagttgaaggtgataataccaaagaaagagacttggatagagccagtgagaaggtggaacctagagatgaagatttggtggtagctcagcaaataaatgcccaaaggcccgagccccagtcagacaatgattccagtgccacgtgcagcgctgatgaggatgtggatggagagccagagaggcagagaatgtttcctatggactcaaagccttcactgttaaaccccactggatctatactcgtctcatctccgttaaaaccaaatccactggatctgccacagcttcagcatcgagctgctgttatcccaccaatggtatcctgcaccccatgtaacataccaattggaaccccagtgagcggctatgctctctaccagcgacacattaaagcaatgcatgagtcagcactcctggaggagcagcggcagagacaagaacagatagatttggaatgtagaagttctacaagtccatgtggcacatccaagagtccaaacagagagtgggaagtccttcagcctgctccacatcaagtgataactaatctccctgaaggcgttcggcttccgacaactcgaccaaccaggccaccgccccctctcatcccgtcatccaaaaccacagtggcttcagaaaaaccatcttttataatgggaggctccatctcacagggaacaccaggcacttatttgacttctcataatcaggcttcctacactcaagaaacacccaagccgtcagtgggatctatctctcttggactgccacggcaacaggaatctgccaaatcagctactttgccctacatcaagcaggaagaattttctccccgaagccaaaactcacaacctgagggtctgttggtcagggcccaacatgaaggtgtagtcagaggtaccgcaggagccatacaagaaggaagtataactcggggaactccaaccagcaaaatttcagtggagagcattccatccctacggggctctatcactcagggcaccccggctctgccccagactggcataccaacagaggctttggtgaaggggtccatttcgagaatgcccattgaagacagcagtcctgagaaaggcagagaggaagctgcatccaaaggccatgttatttatgaaggcaaaagtggacatatcttgtcatatgataatattaagaatgcccgagaagggactaggagtccaagaacagctcatgaaatcagtttaaagagaagctatgaatcagtggaaggaaatataaagcaagggatgtcaatgagggagtctcctgtatcagcaccgttagaggggctgatatgccgagcattacccagggggagtcctcattctgacctcaaagaaaggactgtattgtctggctccataatgcaggggacaccaagagcaacaactgaaagctttgaagatggccttaaatatcccaaacaaattaaaagggaaagtcctcccatacgagcatttgaaggtgccattaccaaaggaaaaccatatgatggcatcaccaccatcaaagaaatggggcgttccattcatgagattccaaggcaagatattttaactcaggaaagtcggaaaactccagaagtggtccagagcacacggccgataattgagggttccatttcccagggcacaccaataaagtttgacaacaactcaggtcaatctgccatcaaacacaatgtcaaatccttaatcacggggcctagcaaactatcccgtggaatgcctccgctggaaattgtgccagagaacataaaagtggtagaacggggaaaatatgaggatgtgaaagcaggcgagaccgtgcgttcccggcacacgtcagtggtaagctctggcccctccgttcttaggtccacactgcatgaagctcccaaagcacaactgagccctgggatttatgatgacaccagtgcacggaggacccctgtgagttatcaaaacaccatgtccagaggctcacccatgatgaacagaacttctgatgttacaatttcttctaacaagtctaccaatcatgaaaggaaatcgacactgacccctacccagagggaaagtatcccagcgaagtctccagtgcctggggtggaccctgtcgtgagccacagtccgtttgatccccatcacagaggcagcactgcaggcgaggtttatcggagccacctgcccacgcacttggatccagccatgccttttcacagggctttggatcctgcagcggctgcttacctgtttcagagacagctttcaccaactccaggttacccaagtcagtatcagctttacgcaatggagaacacaagacagacaatcttaaatgattacattacctcacaacagatgcaagtgaacttgcgtccagatgtggccagaggactctccccaagagagcagccactgggtctcccatacccagcaacgagaggaatcattgacctgaccaatatgcctccaacaattttagtgcctcatccagggggaacaagcactcctcccatggacagaatcacttatattcctggtacacagattactttccctcccaggccgtacaactctgcttccatgtctccaggacacccaacacaccttgcagctgctgcaagtgctgagagggaacgggaacgggagcgggagaaggagcgggagcgggaacggattgctgcagcttcctccgacctctacctgcggccaggctcagaacagcctggccgacctggcagtcatggatatgttcgctccccttccccttcagtaagaactcaggagaccatgttgcaacagagacccagtgttttccaaggaaccaatggaaccagtgtaatcacacctttggatccaactgctcagctacgaatcatgccactgcctgctgggggcccttcaataagccaaggcctgccagcctcccgttacaacactgctgcggatgccctggctgctcttgtggatgctgcagcttctgcaccccagatggatgtgtccaaaacaaaagagagtaagcatgaagctgccaggttagaagaaaatttgagaagcaggtcagcagcagttagtgaacagcagcagctagagcagaaaaccctggaggtggagaagagatctgttcagtgtttatacacttcttcagcctttccaagtggcaagccccagcctcattcttcagtagtttattctgaggctgggaaagataaagggcctcctccaaaatccagatatgaggaagagctaaggaccagagggaagactaccattactgcagctaacttcatagacgtgatcatcacccggcaaattgcctcggacaaggatgcgagggaacgtggctctcaaagttcagactcttctagtagcttatcttctcacaggtatgaaacacctagcgatgctattgaggtgataagtcctgccagctcacctgcgccaccccaggagaaactgcagacctatcagccagaggttgttaaggcaaatcaagcggaaaatgatcctaccagacaatatgaaggaccattacatcactatcgaccacagcaggaatcaccatctccccaacaacagctgcccccttcttcacaggcagagggaatggggcaagtgcccaggacccatcggctgatcacacttgctgatcacatctgtcaaattatcacacaagattttgctagaaatcaagtttcctcgcagactccccagcagcctcctacttctacattccagaactcaccttctgctttggtatctacacctgtgaggactaaaacatcaaaccgttacagcccagaatcccaggctcagtctgtccatcatcaaagaccaggttcaagggtctctccagaaaatcttgtggacaaatccaggggaagtaggcctggaaaatccccagagaggagtcacgtctcttcggagccctacgagcccatctccccaccccaggttccggttgtgcatgagaaacaggacagcttgctgctcttgtctcagaggggcgcagagcctgcagagcagaggaatgatgcccgctcaccagggagtataagctacttgccttcattcttcaccaagcttgaaaatacatcacccatggttaaatcaaagaagcaggagatttttcgtaagttgaactcctctggtggaggtgactctgatatggcagctgctcagccaggaactgagatctttaatctgccagcagttactacgtcaggctcagttagctctagaggccattcttttgctgatcctgccagtaatcttgggctggaagacattatcaggaaggctctcatgggaagctttgatgacaaagttgaggatcatggagttgtcatgtcccagcctatgggagtagtgcctggtactgccaacacctcagttgtgaccagtggtgagacacgaagagaggaaggggacccatcacctcattcaggaggagtttgcaaaccaaagctgatcagcaagtcaaacagcaggaaatctaagtctcctatacctgggcaaggctacttaggaacggaacggccctcttcagtctcctctgtacattcagaaggggattaccataggcagacgccagggtgggcctgggaagacaggccctcttcaacaggctcaactcagtttccttataaccctctgactatgcggatgctcagcagtactccaccaacaccgattgcatgtgctccctctgcggtgaaccaagcagctcctcaccaacagaacaggatctgggagcgagagcctgccccactgctctcagcacagtacgagaccctgtcggatagtgatgactgaactgcacaaagtgaggggaacagggtgcaggagagggatctctagtttttgtggtttaatttttagtagcaggtcaaaaacctgccctcctgtgacttattccctgagacttttcaggagagccagcccacagatgatgaagaaatgatggaagttcatttggagagtcaaatgggaaaaaaacaaacaaaaaactgcctttgatacaggcaattcagtggactataataatagtggagggttgagatgtagagtttttaaaaagtgaacagttgctgttcttacatctgtaaagaaaaccataatgtctttaaatcactcttctgtaaatagatgacctttttgcagtgtatatccccttgctgtagtatctggtgtacttatgttcaaatcagcgcatcaactttgggggtgatttttaaaaatctttttgtctatctatctttttaaccctagccttctaaacaacctcatacagcccagttacataatgttggctgtcacgggcattgtacttttatctgatattgtttcctctaaattcagctttccagtgatgtttaaaatcttgtgaaaatgtttagatttttaacacagaccctgtcataaaatctgtacattagggtcaaaaggtaaaagtaacaaattctgccatattgtaaatttccagtgcaggctttaatttttttttttcattagtagcactgaaaaaatattactgcatgggtatgttctagttcagtttataaagttttaaaggcttatttgaggcatacctcactgttacgcacactggtaatttaaccatgcccctaagtattccttttctcctgcatttgatgcagcccaacaaagcttttgttttgaaataaatttgactaccctgtccatagctacagtagattatttgtggtttaaggctcctggtgtctcaggttccaaaggaaaagcttacatatttttcccttagtttgaatatatgattggttgggttaaaagataatgatctgtgtagtatttagataagctttatgctgcatcctgaaaaactcatggtgaacacagtcctttttccccatcactatggaccagcatttactctcactttgctcccttgggacaagagtttactgttaaatgttttcatttcacagagtctcaaggtgcaaataatttaaaagactgaattctaaactaattatggtactagagggccagttttatctttcattaagaattgcttgctgaattttaaagtttttttcatacaatttatcatagcatttaagtatctttctataacatagatactaacagttttgggagaatgccactggtaactggaaaggggagaaacagatctctcaggatgataaaaattagcactttacagactttcaagtagacctaaacttttaaacaaaagtactcaaggcttttaaggaagcagctctgtgattagctactgaccaagaccctcctatcactggtgtctaatccctatgttacagatgaagacacaggtttagtactttgcccatatagttaaattagtgacagagataggccataagcccacatttgtcttcagtcaaagctttcactcctgtccctgttccactcctgtatacctgaggtccccaacataaactttagatcaggcttagtggtcagcattcctagtacttggaaagttggtattttttacaacagatatatgtaaacatataaaaatttcaaaatgaatgaaaaacagtgactaaatgttccacttcacagttttctgctgaatttttttttttcaggtactggtaatattttagagtttgttaataatttatattgccaacctaccataaaagagattatgatggtatttttctatgaccctgagggtcttaagctattctgagtcagaatacagttgacccttgaacaacacgggtttgaactgtgtgggtccacttatacatggattttcttccacctctgccacccaagatagcaagaccaaccccttctcatcctcagcctattcaacatgaagatgacaaggatgaagaccttcatgatgatccacttccacttaatgaatagtaaatatattttctcttccttataatcttaacaaacattttctcttctctagcttactttattgtaagaatacagtatataatacatatacaaaatatgtgtcaatcgactatgttatccgtaagacttctggtcagcagtaggccatgagtagttaagttttggaggagtcaaagttagatgtggatttttgactacttagggggtctatgcccctaactcctgcattatttgaaggtcaaccgtatagtttctgagaacatgtcagatcagagcctaacttcttactcttgcagttctacctgaggacccgaaggtgaggtgctgtgaaccactccctccatcctcatcataatgcatgtgcgtattactttatttaccatcacctggagaagaaatctcagaatgagagttaaaatccaataatagttcttggttcaaaataaatgaaaggacagaattctcacatggtttacctatggaggttcaccttatcatgatttaagtggtaagcacagagcagcactgacaacttactgtggttcactgcaaaccaaaacatgatattctttgtagacttagatttcagtgttctgacactatatccttacacgtgatttttttagcacccagaactaaattaaggtcttcctaatactcttcataaaagtattctgacagccagttttccactgcatgtttatgactgcaaatttcataatgcttttaattttggtacactgagtgattctattttaaaaatatttatgcccagaaaactccagtgaccattggacagttaaaaaattttaatgacacgattttaaccaattcacttttgaatttagaccaatcagtaaaggagatcagtcctccttttgcttcagtaccttattttacaacagtgtaaatgaactgtaatgaaaacacccacatgaagagactgtcctgaaaactcctagtgtttccaacaacgagaccatgagctgagtcactgtggatgatttaaaattaataaataaataaaacttggaatgaaaaa

SEQ ID NO: 16 - DNA, 8772 bp, Homo sapiens
atgctcagtggcttctcgacaagttggcagcaacaacacggccctggtcgtcgtcgccgctgcggtaacggagcggtttgggtggcggagcctgcgttcgcgccttcccgctctcctcgggaggcccttcctgctctcccctaggctccgcggccgcccagggggtgggagcgggtgaggggagccaggcgcccagcgagagaggccccccgccgcagggcggcccgggagctcgaggcggtccggcccgcgcgggcagcggcgcggcgctgaggaggggcggcctggccgggacgcctcggggcgggggccgaggagctctccgggccgccggggaaagctacgggcccggtgcgtccgcggaccagcagcgcgggagagcggactcccctcgccaccgcccgagcccaggttatcctgaatacatgtctaacaattttccttgcaacgttagctgttgtttttcactgtttccaaaggatcaaaattgcttcagaaattggagacatatttgatttaaaaggaaaaacttgaacaaatggacaatatgtctattacgaatacaccaacaagtaatgatgcctgtctgagcattgtgcatagtttgatgtgccatagacaaggtggagagagtgaaacatttgcaaaaagagcaattgaaagtttggtaaagaagctgaaggagaaaaaagatgaattggattctttaataacagctataactacaaatggagctcatcctagtaaatgtgttaccatacagagaacattggatgggaggcttcaggtggctggtcggaaaggatttcctcatgtgatctatgcccgtctctggaggtggcctgatcttcacaaaaatgaactaaaacatgttaaatattgtcagtatgcgtttgacttaaaatgtgatagtgtctgtgtgaatccatatcactacgaacgagttgtatcacctggaattgatctctcaggattaacactgcagagtaatgctccatcaagtatgatggtgaaggatgaatatgtgcatgactttgagggacagccatcgttgtccactgaaggacattcaattcaaaccatccagcatccaccaagtaatcgtgcatcgacagagacatacagcaccccagctctgttagccccatctgagtctaatgctaccagcactgccaactttcccaacattcctgtggcttccacaagtcagcctgccagtatactggggggcagccatagtgaaggactgttgcagatagcatcagggcctcagccaggacagcagcagaatggatttactggtcagccagctacttaccatcataacagcactaccacctggactggaagtaggactgcaccatacacacctaatttgcctcaccaccaaaacggccatcttcagcaccacccgcctatgccgccccatcccggacattactggcctgttcacaatgagcttgcattccagcctcccatttccaatcatcctgctcctgagtattggtgttccattgcttactttgaaatggatgttcaggtaggagagacatttaaggttccttcaagctgccctattgttactgttgatggatacgtggacccttctggaggagatcgcttttgtttgggtcaactctccaatgtccacaggacagaagccattgagagagcaaggttgcacataggcaaaggtgtgcagttggaatgtaaaggtgaaggtgatgtttgggtcaggtgccttagtgaccacgcggtctttgtacagagttactacttagacagagaagctgggcgtgcacctggagatgctgttcataagatctacccaagtgcatatataaaggtctttgatttgcgtcagtgtcatcgacagatgcagcagcaggcggctactgcacaagctgcagcagctgcccaggcagcagccgtggcaggaaacatccctggcccaggatcagtaggtggaatagctccagctatcagtctgtcagctgctgctggaattggtgttgatgaccttcgtcgcttatgcatactcaggatgagttttgtgaaaggctggggaccggattacccaagacagagcatcaaagaaacaccttgctggattgaaattcacttacaccgggccctccagctcctagacgaagtacttcataccatgccgattgcagacccacaacctttagactgaggtcttttaccgttggggcccttaaccttatcaggatggtggactacaaaatacaatcctgtttataatctgaagatatatttcacttttgttctgctttatcttttcataaagggttgaaaatgtgtttgctgccttgctcctagcagacagaaactggattaaaacaattttttttttcctcttcagaacttgtcaggcatggctcagagcttgaagattaggagaaacacattcttattaattcttcacctgttatgtatgaaggaatcattccagtgctagaaaatttagccctttaaaacgtcttagagccttttatctgcagaacatcgatatgtatatcattctacagaataatccagtattgctgattttaaaggcagagaagttctcaaagttaattcacctatgttattttgtgtacaagttgttattgttgaacatacttcaaaaataatgtgccatgtgggtgagttaattttaccaagagtaactttactctgtgtttaaaaagtaagttaataatgtattgtaatctttcatccaaaatattttttgcaagttatattagtgaagatggtttcaattcagattgtcttgcaacttcagttttatttttgccaaggcaaaaaactcttaatctgtgtgtatattgagaatcccttaaaattaccagacaaaaaaatttaaaattacgtttgttattcctagtggatgactgttgatgaagtatacttttcccctgttaaacagtagttgtattcttctgtatttctaggcacaaggttggttgctaagaagcctataagaggaatttcttttccttcattcatagggaaaggttttgtattttttaaaacactaaaagcagcgtcactctacctaatgtctcactgttctgcaaaggtggcaatgcttaaactaaataatgaataaactgaatattttggaaactgctaaattctatgttaaatactgtgcagaataatggaaacattacagttcataataggtagtttggatatttttgtacttgatttgatgtgactttttttggtataatgtttaaatcatgtatgttatgatattgtttaaaattcagtttttgtatcttggggcaagactgcaaacttttttatatcttttggttattctaagccctttgccatcaatgatcatatcaattggcagtgactttgtatagagaatttaagtagaaaagttgcagatgtattgactgtaccacagacacaatatgtatgctttttacctagctggtagcataaataaaactgaatctcaacatacaaagttgaattctaggtttgatttttaagattttttttttcttttgcacttttgagtccaatctcagtgatgaggtaccttctactaaatgacaggcaacagccagttctattgggcagctttgtttttttccctcacactctaccgggacttccccatggacattgtgtatcatgtgtagagttggttttttttttttttaatttttattttactatagcagaaatagacctgattatctacaagatgataaatagattgtctacaggataaatagtatgaaataaaatcaaggattatctttcagatgtgtttacttttgcctggagaacttttagctatagaaacacttgtgtgatgatagtcctccttatatcacctggaatgaacacagcttctactgccttgctcagaaggtcttttaaatagaccatcctagaaaccactgagtttgcttatttctgtgatttaaacatagatcttgatccaagctacatgacttttgtctttaaataacttatctaccacctcatttgtactcttgattacttacaaattctttcagtaaacacctaattttcttctgtaaaagtttggtgatttaagttttattggcagttttataaaaagacatcttctctagaaattgctaactttaggtccattttactgtgaatgaggaataggagtgagttttagaataacagatttttaaaaatccagatgatttgattaaaaccttaatcatacattgacataattcattgcttcttttttttgagatatggagtcttgctgtgttgcccaggcaggagtgcagtggtatgatctcagctcactgcaacctctgcctcccgggttcaactgattctcctgcctcagcctccctggtagctaggattacaggtgcccgccaccatgcctggctaacttttgtagttttagtagagacggggttttgcctgttggccaggctggtcttgaactcctgacctcaagtgatccatccaccttggcctcccaaagtgctgggattacgggcgtgagccactgtccctggcctcattgttcccttttctactttaaggaaagttttcatgtttaatcatctggggaaagtatgtgaaaaatatttgttaagaagtatctctttggagccaagccacctgtcttggtttctttctactaagagccataaagtatagaaatacttctagttgttaagtgcttatatttgtacctagatttagtcacacgcttttgagaaaacatctagtatgttatgatcagctattcctgagagcttggttgttaatctatatttctatttcttagtggtagtcatctttgatgaataagactaaagattctcacaggtttaaaattttatgtctactttaagggtaaaattatgaggttatggttctgggtgggttttctctagctaattcatatctcaaagagtctcaaaatgttgaatttcagtgcaagctgaatgagagatgagccatgtacacccaccgtaagacctcattccatgtttgtccagtgcctttcagtgcattatcaaagggaatccttcatggtgttgcctttattttccggggagtagatcgtgggatatagtctatctcatttttaatagtttaccgcccctggtatacaaagataatgacaataaatcactgccatataaccttgctttttccagaaacatggctgttttgtattgctgtaaccactaaataggttgcctataccattcctcctgtgaacagtgcagatttacaggttgcatggtctggcttaaggagagccatacttgagacatgtgagtaaactgaactcatattagctgtgctgcatttcagacttaaaatccatttttgtggggcagggtgtggtgtgtaaaggggggtgtttgtaatacaagttgaaggcaaaataaaatgtcctgtctcccagatgatatacatcttattatttttaaagtttattgctaattgtaggaaggtgagttgcaggtatctttgactatggtcatctggggaaggaaaattttacattttactattaatgctccttaagtgtctatggaggttaaagaataaaatggtaaatgtttctgtgcctggtttgatggtaactggttaatagttactcaccattttatgcagagtcacattagttcacaccctttctgagagccttttgggagaagcagttttattctctgagtggaacagagttctttttgttgataatttctagtttgctcccttcgttattgccaactttactggcattttatttaatgatagcagattgggaaaatggcaaatttaggttacggaggtaaatgagtatatgaaagcaattacctctaaagccagttaacaattattttgtaggtggggtacactcagcttaaagtaatgcatttttttttcccgtaaaggcagaatccatcttgttgcagatagctatctaaataatctcatatcctcttttgcaaagactacagagaataggctatgacaatcttgttcaagcctttccatttttttccctgataactaagtaatttctttgaacataccaagaagtatgtaaaaagtccatggccttattcatccacaaagtggcatcctaggcccagccttatccctagcagttgtcccagtgctgctaggttgcttatcttgtttatctggaatcactgtggagtgaaattttccacatcatccagaattgccttatttaagaagtaaaacgttttaatttttagcctttttttggtggagttatttaatatgtatatcagaggatatactagatggtaacatttctttctgtgcttggctatctttgtggacttcaggggcttctaaaacagacaggactgtgttgcctttactaaatggtctgagacagctatggttttgaatttttagttttttttttttaacccacttcccctcctggtctcttccctctctgataattaccattcatatgtgagtgttagtgtgcctccttttagcattttcttcttctctttctgattcttcatttctgactgcctaggcaaggaaaccagataaccaaacttactagaacgttctttaaaacacaagtacaaactctgggacaggacccaagacactttcctgtgaagtgctgaaaaagacctcattgtattggcatttgatatcagtttgatgtagcttagagtgcttcctgattcttgctgagtttcaggtagttgagatagagagaagtgagtcatattcatattttcccccttagaataatattttgaaaggtttcattgcttccacttgaatgctgctcttacaaaaactggggttacaagggttactaaattagcatcagtagccagaggcaataccgttgtctggaggacaccagcaaacaacacacaacaaagcaaaacaaaccttgggaaactaaggccatttgttttgttttggtgtcccctttgaagccctgccttctggccttactcctgtacagatatttttgacctataggtgcctttatgagaattgagggtctgacatcctgccccaaggagtagctaaagtaattgctagtgttttcagggattttaacatcagactggaatgaatgaatgaaactttttgtcctttttttttctgtttttttttttctaatgtagtaaggactaaggaaaacctttggtgaagacaatcatttctctctgttgatgtggatacttttcacaccgtttatttaaatgctttctcaataggtccagagccagtgttcttgttcaacctgaaagtaatggctctgggttgggccagacagttgcactctctagtttgccctctgccacaaatttgatgtgtgacctttgggcaagtcatttatcttctctgggccttagttgcctcatctgtaaaatgagggagttggagtagattaattattccagctctgaaattctaagtgaccttggctaccttgcagcagttttggatttcttccttatctttgttctgctgtttgagggggctttttacttatttccatgttattcaaaggagactaggcttgatattttattactgttcttttatggacaaaaggttacatagtatgcccttaagacttaattttaaccaaaggcctagcaccaccttaggggctgcaataaacacttaacgcgcgtgcgcacgcgcgcgcgcacacacacacacacacacacacacacacacaggtcagagtttaaggctttcgagtcatgacattctagcttttgaattgcgtgcacacacacacgcacgcacacactctggtcagagtttattaaggctttcgagtcatgacattatagcttttgagttggtgtgtgtgacaccaccctcctaagtggtgtgtgcttgtaattttttttttcagtgaaaatggattgaaaacctgttgttaatgcttagtgatattatgctcaaaacaaggaaattcccttgaaccgtgtcaattaaactggtttatatgactcaagaaaacaataccagtagatgattattaactttattcttggctctttttaggtccattttgattaagtgacttttggctggatcattcagagctctcttctagcctacccttggatgagtacaattaatgaaattcatattttcaaggacctgggagccttccttggggctgggttgagggtggggggttggggagtcctggtagaggccagctttgtggtagctggagaggaagggatgaaaccagctgctgttgcaaaggctgcttgtcattgatagaaggactcacgggcttggattgattaagactaaacatggagttggcaaactttcttcaagtattgagttctgttcaatgcattggacatgtgatttaagggaaaagtgtgaatgcttatagatgatgaaaacctggtgggctgcagagcccagtttagaagaagtgagttgggggttggggacagatttggtggtggtatttcccaactgtttcctcccctaaattcagaggaatgcagctatgccagaagccagagaagagccactcgtagcttctgctttggggacaactggtcagttgaaagtcccaggagttcctttgtggctttctgtatacttttgcctggttaaagtctgtggctaaaaaatagtcgaacctttcttgagaactctgtaacaaagtatgtttttgattaaaagagaaagccaactaaa

SEQ ID NO: 17 - DNA, 7088 bp, Homo sapiens
gccagcgccccggcgaacggcaaagagggagccgctcccgctcggggggccgctggagtgcccagcgggaacccgaaagtttgtaagaggaagagagcgcgcggcgagcgagcgagcgggccgggggcagcggcagcggcgccggggaccatggtgctgccggcgcctcctccgcgggcgtgaaggcggcgctcctactccctccccggactccgcggtgtcccagaagcttttgttgacaattccagtttccgaacaaaacatttcggcaatggtgagggcttcgatcccttctctgatttgctgtcagccatgaacggatggatgtgatgcctgctagccaaaaggcttccctctgtgtgttgcagtcctgtggcattatgcatgccccctcccagtgaccccaggctttttatggctgtgagacacgttaaaatttcaggggtaagacgtgaccttttgaggtgactataactgaagattgctttacagaagccaaaaaaggtttttgagtcatgatgcaagaatctgggactgagacaaaaagtaacggttcagccatccagaatgggtcgggcggcagcaaccacttactagagtgcggcggtcttcgggaggggcggtccaacggagagacgccggccgtggacatcggggcagctgacctcgcccacgcccagcagcagcagcaacaggcacttcaggtggcaagacagctccttcttcagcagcaacagcagcagcaagttagtggattaaaatctcccaagaggaatgacaaacaaccagctcttcaggttcccgtgtcagtggctatgatgacacctcaagttatcactccccagcaaatgcagcagatcctccagcaacaagtgctgagccctcagcagctccaggttctcctccagcagcagcaggccctcatgcttcaacagcagcagcttcaagagttttataaaaaacaacaggaacagttgcagcttcaacttttacaacaacaacatgctggaaaacagcctaaagagcaacagcaggtggctacccagcagttggcttttcagcagcagcttttacagatgcagcagttacagcagcagcacctcctgtctttgcagcgccaaggccttctgacaattcagcccgggcagcctgcccttccccttcaacctcttgctcaaggcatgattccaacagaactgcagcagctctggaaagaagtgacaagtgctcatactgcagaagaaaccacaggcaacaatcacagcagtttggatctgaccacgacatgtgtctcctcctctgcaccttccaagacctccttaataatgaacccacatgcctctaccaatggacagctctcagtccacactcccaaaagggaaagtttgtcccatgaggagcacccccatagccatcctctctatggacatggtgtatgcaagtggccaggctgtgaagcagtgtgcgaagatttccaatcatttctaaaacatctcaacagtgagcatgcgctggacgatagaagtacagcccaatgtagagtacaaatgcaggttgtacagcagttagagctacagcttgcaaaagacaaagaacgcctgcaagccatgatgacccacctgcatgtgaagtctacagaacccaaagccgcccctcagcccttgaatctggtatcaagtgtcactctctccaagtccgcatcggaggcttctccacagagcttacctcatactccaacgaccccaaccgcccccctgactcccgtcacccaaggcccctctgtcatcacaaccaccagcatgcacacggtgggacccatccgcaggcggtactcagacaaatacaacgtgcccatttcgtcagcagatattgcgcagaaccaagaattttataagaacgcagaagttagaccaccatttacatatgcatctttaattaggcaggccattctcgaatctccagaaaagcagctaacactaaatgagatctataactggttcacacgaatgtttgcttacttccgacgcaacgcggccacgtggaagaatgcagtgcgtcataatcttagtcttcacaagtgttttgtgcgagtagaaaacgttaaaggggcagtatggacagtggatgaagtagaattccaaaaacgaaggccacaaaagatcagtggtaacccttcccttattaaaaacatgcagagcagccacgcctactgcacacctctcaatgcagctttacaggcttcaatggctgagaatagtatacctctatacactaccgcttccatgggaaatcccactctgggcaacttagccagcgcaatacgggaagagctgaacggggcaatggagcataccaacagcaacgagagtgacagcagtccaggcagatctcctatgcaagccgtgcatcctgtacacgtcaaagaagagcccctcgatccagaggaagctgaagggcccctgtccttagtgacaacagccaaccacagtccagattttgaccatgacagagattacgaagatgaaccagtaaacgaggacatggagtgactatcggggcgggccaaccccgagaatgaagattggaaaaaggaaaaaaaaaaaaacacgtcaaaagttagcagtgaaattgttctccatttgttgtacagtctggaggattttcactacgttttgacaactctgaaatgtgttaactcttagtgccatcaagaaccccatttgggagtatttttgatttttctactttttgttgaaaaaaggaatttgtactctgtgcattggatggacttgtttggtacttgggattttcctctcttaaccgtcaacatcagtgttgtaaatttgctaaactgattcacttttagcagcagactttgaactgcagtcctgccaacgttggacactgaggacgcccgacagagcttgtgcacctaagctgcagaccaagcctttgcccagaatttaaggattccaatggacgacctatttgcacagtactgcatgttgattatcactgcctttactccttttttttttttttttttttttttttttttgcttccagttgggatggggaaggcctttgtgtgtgtattggggggaggggttaaaaaataattatcccaaactttttaatgtattgctttttttttttttttttttccttctactataccattttaagttctgacctcaggcctccatttgggccgatggcctcttggaggcttaaagttttctgtaccttgtgatgaatgttaataggtgtttttattatacaaagctgaatgtcatttctcgtttgtagctttctgtcactcattccatcttccttcagacatcaccacgtttctctaaagtcagaaaacattccgttttggtctttttcaaaaaggtcccaaatgctgcactctacacatgaaggccctctcacacagacgtgacgtcctgccagaaagagaatgaatgacagaaaaaaaaaagagagacaaactctaggaacaatgccgattcattccacgcagcagtattgggggtggttcgggggaggggtgtttcggattttctttttttcttttcttttctttttttttttttgcagcaaccattaataaatgccaccacattctaccagcacaaggaaacataggcagcactgaaaaaaaaaaaaaagctcatattaattagactgacaatatggccttggaaggctctcccttgtggaaccaagttgccatgggccttgggtgctctgcgataacgggtgtgggttggttttgtttgcaaaatggccaaaaaaaaaaaccggcttccccgagcagctgccctgaaagtaggggtggcggcggcggcgctgagtttatacattagttcagacctacttggtggcattaaactgtttgaatgcaaattcgatttcagattgaacttgttaagggagttaacgagggctgagttcagcaaatgctaaagtgttaatttcaaatatgcaaatttggtactgcagtttgttatgcaatattatatcaccaacccagtatcacaaaaactcatagaagatatcatgtaggccctgggctttgggggggtcccaaacatggtatgcagaaatgtgatggttacaggtcagtacaacctcagtccttagaacccctccacacttcagctctgcacccactttcctgtcatttatttatataggactgtagttttttttagttcgagagcctttcgaagcttaatttatattctttctttgtaccttttttctaaaattaccaaagatattacacaaaggtaaattatgttctctgttttatgctttatctgatgaagccaaatatcctcttattgttgatcaaaggaggcaaaagaatttagaggcaaatgacaagcgataggctattgcaacctgagaaagagaactgctccttcatcgtaaatttagaagaccaagtagataatggaaccaaagttgttacttttttctagtagttatttttcctttttctttttgtgtacctctacagagaccaaaactcattctcttaaagagattttatggggctactgcagataaaaataggacacaatattaaaggagctacagaaggaagggagtcccatctcaaaaaaaaaatgaatgtatgccactgcaattagagtatccaataaaggagacagtttagagtcaggacagaaaagcttccataattgaactagattacataatagtatttctagaaaaagagatatttttagattgtatgccacttttgtttaagaactgtgctgtgatcactgtattaattttggtttatcttggcatatatccttcagtttgtttttatttttaatttttcctttttttccgattaggctttggtcagcatttttcatttaaagaaaagtaacactcccatccactcataagcttggtacaaaaacttctctggcagttacttttgaagcttcactctgctttctgtataaagggcagtctgtggtcacgcaagactttaaaaaaaaaaaaaaaaaaaaaaaaaaaaaaaaaaaaaaaaacttttccaggcagcttcatgatgtgcaggcagtagccagacagggtcatgggaagggggccctgtgcttctaaactgagtggttgctggttagtttggtattcaaaagaggataaaaatctggtagattagttcattctcagcatgtgtagctagacatgagtaaagataacagcatgagaaactgttagtacgcatacctcagttcaaacctttagggaatgattaaaatttaaaaaaaaaacatttcactcagttgcacttagtcgtatgtcttgcatgcttagtctaaagactgtagcaaaaaaaaaaaaaaaagaaaaattagattttacatatctttgcaggtatcacagccttgcagaagaaccaactgaaaaaaaaattctcaggctttacagcaagcaaacttcactatgatttttacaattctgattctgtatcccctgggggttatcccagttgcttctttaggatggggtttattacgttgtacatatatcccgatgtgtctgtgtgaatctttgtcttttttgggggagggcagagggcggttctttttttagatattgttcctaaaaaggaataaatgcatacacctgtttgtcaaaacacctttgctttttgtgcaactgctttatattaacgatactaaaaaaaaatagctttggaaaaaaaactactgtatgtaacggaattgcagaatatgctgcacatgtattttatttagttatccttgctttaagaatattggatgacatttcctgacatgtgggagggagaaactccctaactttttttttctgcttttaaactgtaacatagttgaagatttcttttttctgttctcattgattggagcattttgtacaggttttgtgtgtgtgtgtgtgtgtgtgtgcgcgcgtgcgtgtgtgttaatctgttttttgatacattcctatcccttgtgtttatcctaccactgccttcctggctatcttaaacaagttcatacatttgaaaagaaaaaaaaatgttgtttaaaaaatgttttctcctgctgcagtaaatattttgcatgatgaaattccagggtcacacttttccaagtttatcagtgaagtagtgattaacaatggggagtgtcaaaactattgaacttttgtataaaaaaaaaaaaactttacaaggtgccaagatgtaaagaaaatctgttactttttttttctcaaagaaaagcatacattagggaggtagtcccgtgtgtcagacaaatgcactgtcaggaatgaggatccaacctacctgtccctagagtccgtcttgtaagatgagttaggctgccccttggaccagccacaaaatggaatatcaaggcttatgtacatacgtgaagagttaccaccagtcctgccacctttggacagctctaacaccatccccagcatccagtcagacctagtaaagaaaaccttggattcttaacccaagataggctgtaaatcactagcttttttttcctcatgaaaaaaaatagagttaaaaaatatttcctctcttttccatattccagctgaactccgtttccaaaggcacaaagaagagtgtgcttattcagattttgaatctttttggtaccttttggttaatgacatagcctcctgaaattctggatgtcttcaaagtcagttttgcttctttatcctgaaaatcagatttacaatgctgaaggcatttcttgggcccagtgtagctcacgcaatctctgctacccataagccttgatgaagatgatacagtccggactgtgagcatggtgcttcatgtatatgtgctgccagtaacaagaatttttttgttttgttttgttttgttttgataaggcataaaagaaactcattccttgacatcaactgtaattccatcattccatgtctgcggatacagacaataaaaaaaatgttgtgtagtcagtactaattactgacattataagcattctcaaatgcaataaaaatgctggttgttcacgctggtagtaaaa

SEQ ID NO: 18 - DNA, 10704 bp, Homo sapiens
actggagacagaatggaggtgctgccggactcggaaatggggtccaagggtagccaaggatggctgcagcttcatatgatcagttgttaaagcaagttgaggcactgaagatggagaactcaaatcttcgacaagagctagaagataattccaatcatcttacaaaactggaaactgaggcatctaatatgaaggaagtacttaaacaactacaaggaagtattgaagatgaagctatggcttcttctggacagattgatttattagagcgtcttaaagagcttaacttagatagcagtaatttccctggagtaaaactgcggtcaaaaatgtccctccgttcttatggaagccgggaaggatctgtatcaagccgttctggagagtgcagtcctgttcctatgggttcatttccaagaagagggtttgtaaatggaagcagagaaagtactggatatttagaagaacttgagaaagagaggtcattgcttcttgctgatcttgacaaagaagaaaaggaaaaagactggtattacgctcaacttcagaatctcactaaaagaatagatagtcttcctttaactgaaaatttttccttacaaacagatatgaccagaaggcaattggaatatgaagcaaggcaaatcagagttgcgatggaagaacaactaggtacctgccaggatatggaaaaacgagcacagcgaagaatagccagaattcagcaaatcgaaaaggacatacttcgtatacgacagcttttacagtcccaagcaacagaagcagagaggtcatctcagaacaagcatgaaaccggctcacatgatgctgagcggcagaatgaaggtcaaggagtgggagaaatcaacatggcaacttctggtaatggtcagggttcaactacacgaatggaccatgaaacagccagtgttttgagttctagtagcacacactctgcacctcgaaggctgacaagtcatctgggaaccaaggtggaaatggtgtattcattgttgtcaatgcttggtactcatgataaggatgatatgtcgcgaactttgctagctatgtctagctcccaagacagctgtatatccatgcgacagtctggatgtcttcctctcctcatccagcttttacatggcaatgacaaagactctgtattgttgggaaattcccggggcagtaaagaggctcgggccagggccagtgcagcactccacaacatcattcactcacagcctgatgacaagagaggcaggcgtgaaatccgagtccttcatcttttggaacagatacgcgcttactgtgaaacctgttgggagtggcaggaagctcatgaaccaggcatggaccaggacaaaaatccaatgccagctcctgttgaacatcagatctgtcctgctgtgtgtgttctaatgaaactttcatttgatgaagagcatagacatgcaatgaatgaactagggggactacaggccattgcagaattattgcaagtggactgtgaaatgtatgggcttactaatgaccactacagtattacactaagacgatatgctggaatggctttgacaaacttgacttttggagatgtagccaacaaggctacgctatgctctatgaaaggctgcatgagagcacttgtggcccaactaaaatctgaaagtgaagacttacagcaggttattgcgagtgttttgaggaatttgtcttggcgagcagatgtaaatagtaaaaagacgttgcgagaagttggaagtgtgaaagcattgatggaatgtgctttagaagttaaaaaggaatcaaccctcaaaagcgtattgagtgccttatggaatttgtcagcacattgcactgagaataaagctgatatatgtgctgtagatggtgcacttgcatttttggttggcactcttacttaccggagccagacaaacactttagccattattgaaagtggaggtgggatattacggaatgtgtccagcttgatagctacaaatgaggaccacaggcaaatcctaagagagaacaactgtctacaaactttattacaacacttaaaatctcatagtttgacaatagtcagtaatgcatgtggaactttgtggaatctctcagcaagaaatcctaaagaccaggaagcattatgggacatgggggcagttagcatgctcaagaacctcattcattcaaagcacaaaatgattgctatgggaagtgctgcagctttaaggaatctcatggcaaataggcctgcgaagtacaaggatgccaatattatgtctcctggctcaagcttgccatctcttcatgttaggaaacaaaaagccctagaagcagaattagatgctcagcacttatcagaaacttttgacaatatagacaatttaagtcccaaggcatctcatcgtagtaagcagagacacaagcaaagtctctatggtgattatgtttttgacaccaatcgacatgatgataataggtcagacaattttaatactggcaacatgactgtcctttcaccatatttgaatactacagtgttacccagctcctcttcatcaagaggaagcttagatagttctcgttctgaaaaagatagaagtttggagagagaacgcggaattggtctaggcaactaccatccagcaacagaaaatccaggaacttcttcaaagcgaggtttgcagatctccaccactgcagcccagattgccaaagtcatggaagaagtgtcagccattcatacctctcaggaagacagaagttctgggtctaccactgaattacattgtgtgacagatgagagaaatgcacttagaagaagctctgctgcccatacacattcaaacacttacaatttcactaagtcggaaaattcaaataggacatgttctatgccttatgccaaattagaatacaagagatcttcaaatgatagtttaaatagtgtcagtagtagtgatggttatggtaaaagaggtcaaatgaaaccctcgattgaatcctattctgaagatgatgaaagtaagttttgcagttatggtcaatacccagccgacctagcccataaaatacatagtgcaaatcatatggatgataatgatggagaactagatacaccaataaattatagtcttaaatattcagatgagcagttgaactctggaaggcaaagtccttcacagaatgaaagatgggcaagacccaaacacataatagaagatgaaataaaacaaagtgagcaaagacaatcaaggaatcaaagtacaacttatcctgtttatactgagagcactgatgataaacacctcaagttccaaccacattttggacagcaggaatgtgtttctccatacaggtcacggggagccaatggttcagaaacaaatcgagtgggttctaatcatggaattaatcaaaatgtaagccagtctttgtgtcaagaagatgactatgaagatgataagcctaccaattatagtgaacgttactctgaagaagaacagcatgaagaagaagagagaccaacaaattatagcataaaatataatgaagagaaacgtcatgtggatcagcctattgattatagtttaaaatatgccacagatattccttcatcacagaaacagtcattttcattctcaaagagttcatctggacaaagcagtaaaaccgaacatatgtcttcaagcagtgagaatacgtccacaccttcatctaatgccaagaggcagaatcagctccatccaagttctgcacagagtagaagtggtcagcctcaaaaggctgccacttgcaaagtttcttctattaaccaagaaacaatacagacttattgtgtagaagatactccaatatgtttttcaagatgtagttcattatcatctttgtcatcagctgaagatgaaataggatgtaatcagacgacacaggaagcagattctgctaataccctgcaaatagcagaaataaaagaaaagattggaactaggtcagctgaagatcctgtgagcgaagttccagcagtgtcacagcaccctagaaccaaatccagcagactgcagggttctagtttatcttcagaatcagccaggcacaaagctgttgaattttcttcaggagcgaaatctccctccaaaagtggtgctcagacacccaaaagtccacctgaacactatgttcaggagaccccactcatgtttagcagatgtacttctgtcagttcacttgatagttttgagagtcgttcgattgccagctccgttcagagtgaaccatgcagtggaatggtaagtggcattataagccccagtgatcttccagatagccctggacaaaccatgccaccaagcagaagtaaaacacctccaccacctcctcaaacagctcaaaccaagcgagaagtacctaaaaataaagcacctactgctgaaaagagagagagtggacctaagcaagctgcagtaaatgctgcagttcagagggtccaggttcttccagatgctgatactttattacattttgccacggaaagtactccagatggattttcttgttcatccagcctgagtgctctgagcctcgatgagccatttatacagaaagatgtggaattaagaataatgcctccagttcaggaaaatgacaatgggaatgaaacagaatcagagcagcctaaagaatcaaatgaaaaccaagagaaagaggcagaaaaaactattgattctgaaaaggacctattagatgattcagatgatgatgatattgaaatactagaagaatgtattatttctgccatgccaacaaagtcatcacgtaaagcaaaaaagccagcccagactgcttcaaaattacctccacctgtggcaaggaaaccaagtcagctgcctgtgtacaaacttctaccatcacaaaacaggttgcaaccccaaaagcatgttagttttacaccgggggatgatatgccacgggtgtattgtgttgaagggacacctataaacttttccacagctacatctctaagtgatctaacaatcgaatcccctccaaatgagttagctgctggagaaggagttagaggaggggcacagtcaggtgaatttgaaaaacgagataccattcctacagaaggcagaagtacagatgaggctcaaggaggaaaaacctcatctgtaaccatacctgaattggatgacaataaagcagaggaaggtgatattcttgcagaatgcattaattctgctatgcccaaagggaaaagtcacaagcctttccgtgtgaaaaagataatggaccaggtccagcaagcatctgcgtcttcttctgcacccaacaaaaatcagttagatggtaagaaaaagaaaccaacttcaccagtaaaacctataccacaaaatactgaatataggacacgtgtaagaaaaaatgcagactcaaaaaataatttaaatgctgagagagttttctcagacaacaaagattcaaagaaacagaatttgaaaaataattccaaggtcttcaatgataagctcccaaataatgaagatagagtcagaggaagttttgcttttgattcacctcatcattacacgcctattgaaggaactccttactgtttttcacgaaatgattctttgagttctctagattttgatgatgatgatgttgacctttccagggaaaaggctgaattaagaaaggcaaaagaaaataaggaatcagaggctaaagttaccagccacacagaactaacctccaaccaacaatcagctaataagacacaagctattgcaaagcagccaataaatcgaggtcagcctaaacccatacttcagaaacaatccacttttccccagtcatccaaagacataccagacagaggggcagcaactgatgaaaagttacagaattttgctattgaaaatactccggtttgcttttctcataattcctctctgagttctctcagtgacattgaccaagaaaacaacaataaagaaaatgaacctatcaaagagactgagccccctgactcacagggagaaccaagtaaacctcaagcatcaggctatgctcctaaatcatttcatgttgaagataccccagtttgtttctcaagaaacagttctctcagttctcttagtattgactctgaagatgacctgttgcaggaatgtataagctccgcaatgccaaaaaagaaaaagccttcaagactcaagggtgataatgaaaaacatagtcccagaaatatgggtggcatattaggtgaagatctgacacttgatttgaaagatatacagagaccagattcagaacatggtctatcccctgattcagaaaattttgattggaaagctattcaggaaggtgcaaattccatagtaagtagtttacatcaagctgctgctgctgcatgtttatctagacaagcttcgtctgattcagattccatcctttccctgaaatcaggaatctctctgggatcaccatttcatcttacacctgatcaagaagaaaaaccctttacaagtaataaaggcccacgaattctaaaaccaggggagaaaagtacattggaaactaaaaagatagaatctgaaagtaaaggaatcaaaggaggaaaaaaagtttataaaagtttgattactggaaaagttcgatctaattcagaaatttcaggccaaatgaaacagccccttcaagcaaacatgccttcaatctctcgaggcaggacaatgattcatattccaggagttcgaaatagctcctcaagtacaagtcctgtttctaaaaaaggcccaccccttaagactccagcctccaaaagccctagtgaaggtcaaacagccaccacttctcctagaggagccaagccatctgtgaaatcagaattaagccctgttgccaggcagacatcccaaataggtgggtcaagtaaagcaccttctagatcaggatctagagattcgaccccttcaagacctgcccagcaaccattaagtagacctatacagtctcctggccgaaactcaatttcccctggtagaaatggaataagtcctcctaacaaattatctcaacttccaaggacatcatcccctagtactgcttcaactaagtcctcaggttctggaaaaatgtcatatacatctccaggtagacagatgagccaacagaaccttaccaaacaaacaggtttatccaagaatgccagtagtattccaagaagtgagtctgcctccaaaggactaaatcagatgaataatggtaatggagccaataaaaaggtagaactttctagaatgtcttcaactaaatcaagtggaagtgaatctgatagatcagaaagacctgtattagtacgccagtcaactttcatcaaagaagctccaagcccaaccttaagaagaaaattggaggaatctgcttcatttgaatctctttctccatcatctagaccagcttctcccactaggtcccaggcacaaactccagttttaagtccttcccttcctgatatgtctctatccacacattcgtctgttcaggctggtggatggcgaaaactcccacctaatctcagtcccactatagagtataatgatggaagaccagcaaagcgccatgatattgcacggtctcattctgaaagtccttctagacttccaatcaataggtcaggaacctggaaacgtgagcacagcaaacattcatcatcccttcctcgagtaagcacttggagaagaactggaagttcatcttcaattctttctgcttcatcagaatccagtgaaaaagcaaaaagtgaggatgaaaaacatgtgaactctatttcaggaaccaaacaaagtaaagaaaaccaagtatccgcaaaaggaacatggagaaaaataaaagaaaatgaattttctcccacaaatagtacttctcagaccgtttcctcaggtgctacaaatggtgctgaatcaaagactctaatttatcaaatggcacctgctgtttctaaaacagaggatgtttgggtgagaattgaggactgtcccattaacaatcctagatctggaagatctcccacaggtaatactcccccggtgattgacagtgtttcagaaaaggcaaatccaaacattaaagattcaaaagataatcaggcaaaacaaaatgtgggtaatggcagtgttcccatgcgtaccgtgggtttggaaaatcgcctgaactcctttattcaggtggatgcccctgaccaaaaaggaactgagataaaaccaggacaaaataatcctgtccctgtatcagagactaatgaaagttctatagtggaacgtaccccattcagttctagcagctcaagcaaacacagttcacctagtgggactgttgctgccagagtgactccttttaattacaacccaagccctaggaaaagcagcgcagatagcacttcagctcggccatctcagatcccaactccagtgaataacaacacaaagaagcgagattccaaaactgacagcacagaatccagtggaacccaaagtcctaagcgccattctgggtcttaccttgtgacatctgtttaaaagagaggaagaatgaaactaagaaaattctatgttaattacaactgctatatagacattttgtttcaaatgaaactttaaaagactgaaaaattttgtaaataggtttgattcttgttagagggtttttgttctggaagccatatttgatagtatactttgtcttcactggtcttattttgggaggcactcttgatggttaggaaaaaaatagtaaagccaagtatgtttgtacagtatgttttacatgtatttaaagtagcatcccatcccaacttcctttaattattgcttgtcttaaaataatgaacactacagatagaaaatatgatatattgctgttatcaatcatttctagattataaactgactaaacttacatcagggaaaaattggtatttatgcaaaaaaaaatgtttttgtccttgtgagtccatctaacatcataattaatcatgtggctgtgaaattcacagtaatatggttcccgatgaacaagtttacccagcctgctttgctttactgcatgaatgaaactgatggttcaatttcagaagtaatgattaacagttatgtggtcacatgatgtgcatagagatagctacagtgtaataatttacactattttgtgctccaaacaaaacaaaaatctgtgtaactgtaaaacattgaatgaaactattttacctgaactagattttatctgaaagtaggtagaatttttgctatgctgtaatttgttgtatattctggtatttgaggtgagatggctgctcttttattaatgagacatgaattgtgtctcaacagaaactaaatgaacatttcagaataaattattgctgtatgtaaactgttactgaaattggtatttgtttgaagggtcttgtttcacatttgtattaataattgtttaaaatgcctcttttaaaagcttatataaatttttttcttcagcttctatgcattaagagtaaaattcctcttactgtaataaaaacaattgaagaagactgttgccacttaaccattccatgcgttggcacttatctattcctgaaatttcttttatgtgattagctcatcttgatttttaatatttttccacttaaacttttttttcttactccactggagctcagtaaaagtaaattcatgtaatagcaatgcaagcagcctagcacagactaagcattgagcataataggcccacataatttcctctttcttaatattatagaattctgtacttgaaattgattcttagacattgcagtctcttcgaggctttacagtgtaaactgtcttgccccttcatcttcttgttgcaactgggtctgacatgaacactttttatcaccctgtatgttagggcaagatctcagcagtgaagtataatcagcactttgccatgctcagaaaattcaaatcacatggaactttagaggtagatttaatacgattaagatattcagaagtatattttagaatccctgcctgttaaggaaactttatttgtggtaggtacagttctggggtacatgttaagtgtccccttatacagtggagggaagtcttccttcctgaaggaaaataaactgacacttattaactaagataatttacttaatatatcttccctgatttgttttaaaagatcagagggtgactgatgatacatgcatacatatttgttgaataaatgaaaatttatttttagtgataagattcatacactctgtatttggggagggaaaacctttttaagcatggtggggcactcagataggagtgaatacacctacctggtgccttgaaaatcacatcaagtagttaattatctaccccttacctgtgtttataacttccaggtaatgagaatgattttttttaaagctaaaatgccagtaaataaaagtgctatgacttgagctaagatatttgactccaatgcctgtactgtgtctactgcaccactttgtaaacacttcaatttactatctttgaaatgattgacctttaaatttttgccaaatgttatctgaaattgtctatgaataccatctacttctgttgttttcccaggcttccataaacaatggagatacatgca

SEQ ID NO: 19 - DNA, 3931 bp, Homo sapiens
ggagagccgaaagcggagctcgaaactgactggaaacttcagtggcgcggagactcgccagtttcaaccccggaaacttttctttgcaggaggagaagagaaggggtgcaagcgcccccacttttgctctttttcctcccctcctcctcctctccaattcgcctccccccacttggagcgggcagctgtgaactggccaccccgcgccttcctaagtgctcgccgcggtagccggccgacgcgccagcttccccgggagccgcttgctccgcatccgggcagccgaggggagaggagcccgcgcctcgagtccccgagccgccgcggcttctcgcctttcccggccaccagccccctgccccgggcccgcgtatgaatctcctggaccccttcatgaagatgaccgacgagcaggagaagggcctgtccggcgcccccagccccaccatgtccgaggactccgcgggctcgccctgcccgtcgggctccggctcggacaccgagaacacgcggccccaggagaacacgttccccaagggcgagcccgatctgaagaaggagagcgaggaggacaagttccccgtgtgcatccgcgaggcggtcagccaggtgctcaaaggctacgactggacgctggtgcccatgccggtgcgcgtcaacggctccagcaagaacaagccgcacgtcaagcggcccatgaacgccttcatggtgtgggcgcaggcggcgcgcaggaagctcgcggaccagtacccgcacttgcacaacgccgagctcagcaagacgctgggcaagctctggagacttctgaacgagagcgagaagcggcccttcgtggaggaggcggagcggctgcgcgtgcagcacaagaaggaccacccggattacaagtaccagccgcggcggaggaagtcggtgaagaacgggcaggcggaggcagaggaggccacggagcagacgcacatctcccccaacgccatcttcaaggcgctgcaggccgactcgccacactcctcctccggcatgagcgaggtgcactcccccggcgagcactcggggcaatcccagggcccaccgaccccacccaccacccccaaaaccgacgtgcagccgggcaaggctgacctgaagcgagaggggcgccccttgccagaggggggcagacagccccctatcgacttccgcgacgtggacatcggcgagctgagcagcgacgtcatctccaacatcgagaccttcgatgtcaacgagtttgaccagtacctgccgcccaacggccacccgggggtgccggccacgcacggccaggtcacctacacgggcagctacggcatcagcagcaccgcggccaccccggcgagcgcgggccacgtgtggatgtccaagcagcaggcgccgccgccacccccgcagcagcccccacaggccccgccggccccgcaggcgcccccgcagccgcaggcggcgcccccacagcagccggcggcacccccgcagcagccacaggcgcacacgctgaccacgctgagcagcgagccgggccagtcccagcgaacgcacatcaagacggagcagctgagccccagccactacagcgagcagcagcagcactcgccccaacagatcgcctacagccccttcaacctcccacactacagcccctcctacccgcccatcacccgctcacagtacgactacaccgaccaccagaactccagctcctactacagccacgcggcaggccagggcaccggcctctactccaccttcacctacatgaaccccgctcagcgccccatgtacacccccatcgccgacacctctggggtcccttccatcccgcagacccacagcccccagcactgggaacaacccgtctacacacagctcactcgaccttgaggaggcctcccacgaagggcgaagatggccgagatgatcctaaaaataaccgaagaaagagaggaccaaccagaattccctttggacatttgtgtttttttgtttttttattttgttttgttttttcttcttcttcttcttccttaaagacatttaagctaaaggcaactcgtacccaaatttccaagacacaaacatgacctatccaagcgcattacccacttgtggccaatcagtggccaggccaaccttggctaaatggagcagcgaaatcaacgagaaactggactttttaaaccctcttcagagcaagcgtggaggatgatggagaatcgtgtgatcagtgtgctaaatctctctgcctgtttggactttgtaattatttttttagcagtaattaaagaaaaaagtcctctgtgaggaatattctctattttaaatatttttagtatgtactgtgtatgattcattaccattttgaggggatttatacatatttttagataaaattaaatgctcttatttttccaacagctaaactactcttagttgaacagtgtgccctagcttttcttgcaaccagagtatttttgtacagatttgctttctcttacaaaaagaaaaaaaaaatcctgttgtattaacatttaaaaacagaattgtgttatgtgatcagttttgggggttaactttgcttaattcctcaggctttgcgatttaaggaggagctgccttaaaaaaaaataaaggccttattttgcaattatgggagtaaacaatagtctagagaagcatttggtaagctttatcatatatatattttttaaagaagagaaaaacaccttgagccttaaaacggtgctgctgggaaacatttgcactcttttagtgcatttcctcctgcctttgcttgttcactgcagtcttaagaaagaggtaaaaggcaagcaaaggagatgaaatctgttctgggaatgtttcagcagccaataagtgcccgagcacactgcccccggttgcctgcctgggccccatgtggaaggcagatgcctgctcgctctgtcacctgtgcctctcagaacaccagcagttaaccttcaagacattccacttgctaaaattatttattttgtaaggagaggttttaattaaaacaaaaaaaaattcttttttttttttttttccaattttaccttctttaaaataggttgttggagctttcctcaaagggtatggtcatctgttgttaaattatgttcttaactgtaaccagtttttttttatttatctctttaatctttttttattattaaaagcaagtttctttgtattcctcaccctagatttgtataaatgcctttttgtccatcccttttttctttgttgtttttgttgaaaacaaactggaaacttgtttctttttttgtataaatgagagattgcaaatgtagtgtatcactgagtcatttgcagtgttttctgccacagacctttgggctgccttatattgtgtgtgtgtgtgggtgtgtgtgtgttttgacacaaaaacaatgcaagcatgtgtcatccatatttctctgcatcttctcttggagtgagggaggctacctggaggggatcagcccactgacagaccttaatcttaattactgctgtggctagagagtttgaggattgctttttaaaaaagacagcaaacttttttttttatttaaaaaaagatatattaacagttttagaagtcagtagaataaaatcttaaagcactcataatatggcatccttcaatttctgtataaaagcagatctttttaaaaagatacttctgtaacttaagaaacctggcatttaaatcatattttgtctttaggtaaaagctttggtttgtgttcgtgttttgtttgtttcacttgtttccctcccagccccaaaccttttgttctctccgtgaaacttacctttccctttttctttctcttttttttttttgtatattattgtttacaataaatatacattgcattaaaaa

SEQ ID NO: 20 - DNA, 2914 bp, Homo sapiens
gtgctctgagtttttggtttctgtttcaccttgtgtctgagctggtctgaaggctggttgttcagactgagcttcctgcctgcctgtaccccgccaacagcttcagaagaaggtgactggtggctgcctgaggaataccagtgggcaagagaattagcatttctggagcatctgctgtctgagcagcccctgggtgcgtccactttctgggcacgtgaggttgggccttggccgcctgagcccttgagttggtcacttgaaccttgggaatattgagattatattctcctgccttttaaaaagatggacttcagcagaaatctttatgatattggggaacaactggacagtgaagatctggcctccctcaagttcctgagcctggactacattccgcaaaggaagcaagaacccatcaaggatgccttgatgttattccagagactccaggaaaagagaatgttggaggaaagcaatctgtccttcctgaaggagctgctcttccgaattaatagactggatttgctgattacctacctaaacactagaaaggaggagatggaaagggaacttcagacaccaggcagggctcaaatttctgcctacaggttccacttctgccgcatgagctgggctgaagcaaacagccagtgccagacacagtctgtacctttctggcggagggtcgatcatctattaataagggtcatgctctatcagatttcagaagaagtgagcagatcagaattgaggtcttttaagtttcttttgcaagaggaaatctccaaatgcaaactggatgatgacatgaacctgctggatattttcatagagatggagaagagggtcatcctgggagaaggaaagttggacatcctgaaaagagtctgtgcccaaatcaacaagagcctgctgaagataatcaacgactatgaagaattcagcaaaggggaggagttgtgtggggtaatgacaatctcggactctccaagagaacaggatagtgaatcacagactttggacaaagtttaccaaatgaaaagcaaacctcggggatactgtctgatcatcaacaatcacaattttgcaaaagcacgggagaaagtgcccaaacttcacagcattagggacaggaatggaacacacttggatgcaggggctttgaccacgacctttgaagagcttcattttgagatcaagccccacgatgactgcacagtagagcaaatctatgagattttgaaaatctaccaactcatggaccacagtaacatggactgcttcatctgctgtatcctctcccatggagacaagggcatcatctatggcactgatggacaggaggcccccatctatgagctgacatctcagttcactggtttgaagtgcccttcccttgctggaaaacccaaagtgttttttattcaggcttgtcagggggataactaccagaaaggtatacctgttgagactgattcagaggagcaaccctatttagaaatggatttatcatcacctcaaacgagatatatcccggatgaggctgactttctgctggggatggccactgtgaataactgtgtttcctaccgaaaccctgcagagggaacctggtacatccagtcactttgccagagcctgagagagcgatgtcctcgaggcgatgatattctcaccatcctgactgaagtgaactatgaagtaagcaacaaggatgacaagaaaaacatggggaaacagatgcctcagcctactttcacactaagaaaaaaacttgtcttcccttctgattgatggtgctattttgtttgttttgttttgttttgtttttttgagacagaatctcgctctgtcgcccaggctggagtgcagtggcgtgatctcggctcaccgcaagctccgcctcccgggttcaggccattctcctgcctcagcctcccgagtagctgggactacaggggcccgccaccacacctggctaattttttaaaaatatttttagtagagacagggtttcactgtgttagccagggtggtcttgatctcctgacctcgtgatccacccacctcggcctcccaaagtgctgggattacaggcgtgagccaccgcgcctggccgatggtactatttagatataacactatgtttatttactaattttctagattttctactttattaattgttttgcacttttttataagagctaaagttaaataggatattaacaacaataacactgtctcctttctcttatgcttaaggctttgggaatgtttttagctggtggcaataaataccagacacgtacaaaatccagctatgaatatagagggcttatgattcagattgttatctatcaactataagcccactgttaatattctattaactttaattctctttcaaagctaaattccacactaccacattaaaaaaattagaaagtagccacgtatggtggctcatgtctataatcccagcactttgggaggttgaggtgggaggattgcttgaacccaagaggtcaaggctgcagtgagccatgttcacaccgctgcactcaagcttgggtgacagaacaagaccccgtctcaaaaaaaattttttttttaataaaacaaaatttgtttgaaatcttttaaaaattcaaatgatttttacaagttttaaataagctctccccaaacttgctttatgccttcttattgcttttatgatatatatatgcttggctaactatatttgctttttgctaacaatgctctggggtctttttatgcatttgcatttgctctttcatctctgcttggattattttaaatcattaggaattaagttatctttaaaatttaagtatcttttttcaaaaacattttttaatagaataaaatataatttgatcttattaaa

SEQ ID NO: 21 - DNA, 6073 bp, Homo sapiens
agtctccgggatccccaggcctggaggggggtctgtgcgcggccggctggctctgccccgcgtccggtcccgagcgggcctccctcgggccagcccgatgtgaccgagcccagcggagcctgagcaaggagcgggtccgtcgcggagccggagggcgggaggaacatgacatcgcggagatggtttcacccaaatatcactggtgtggaggcagaaaacctactgttgacaagaggagttgatggcagttttttggcaaggcctagtaaaagtaaccctggagacttcacactttccgttagaagaaatggagctgtcacccacatcaagattcagaacactggtgattactatgacctgtatggaggggagaaatttgccactttggctgagttggtccagtattacatggaacatcacgggcaattaaaagagaagaatggagatgtcattgagcttaaatatcctctgaactgtgcagatcctacctctgaaaggtggtttcatggacatctctctgggaaagaagcagagaaattattaactgaaaaaggaaaacatggtagttttcttgtacgagagagccagagccaccctggagattttgttctttctgtgcgcactggtgatgacaaaggggagagcaatgacggcaagtctaaagtgacccatgttatgattcgctgtcaggaactgaaatacgacgttggtggaggagaacggtttgattctttgacagatcttgtggaacattataagaagaatcctatggtggaaacattgggtacagtactacaactcaagcagccccttaacacgactcgtataaatgctgctgaaatagaaagcagagttcgagaactaagcaaattagctgagaccacagataaagtcaaacaaggcttttgggaagaatttgagacactacaacaacaggagtgcaaacttctctacagccgaaaagagggtcaaaggcaagaaaacaaaaacaaaaatagatataaaaacatcctgccctttgatcataccagggttgtcctacacgatggtgatcccaatgagcctgtttcagattacatcaatgcaaatatcatcatgcctgaatttgaaaccaagtgcaacaattcaaagcccaaaaagagttacattgccacacaaggctgcctgcaaaacacggtgaatgacttttggcggatggtgttccaagaaaactcccgagtgattgtcatgacaacgaaagaagtggagagaggaaagagtaaatgtgtcaaatactggcctgatgagtatgctctaaaagaatatggcgtcatgcgtgttaggaacgtcaaagaaagcgccgctcatgactatacgctaagagaacttaaactttcaaaggttggacaagggaatacggagagaacggtctggcaataccactttcggacctggccggaccacggcgtgcccagcgaccctgggggcgtgctggacttcctggaggaggtgcaccataagcaggagagcatcatggatgcagggccggtcgtggtgcactgcagtgctggaattggccggacagggacgttcattgtgattgatattcttattgacatcatcagagagaaaggtgttgactgcgatattgacgttcccaaaaccatccagatggtgcggtctcagaggtcagggatggtccagacagaagcacagtaccgatttatctatatggcggtccagcattatattgaaacactacagcgcaggattgaagaagagcagaaaagcaagaggaaagggcacgaatatacaaatattaagtattctctagcggaccagacgagtggagatcagagccctctcccgccttgtactccaacgccaccctgtgcagaaatgagagaagacagtgctagagtctatgaaaacgtgggcctgatgcaacagcagaaaagtttcagatgagaaaacctgccaaaacttcagcacagaaatagatgtggactttcaccctctccctaaaaagatcaagaacagacgcaagaaagtttatgtgaagacagaatttggatttggaaggcttgcaatgtggttgactaccttttgataagcaaaatttgaaaccatttaaagaccactgtattttaactcaacaatacctgcttcccaattactcatttcctcagataagaagaaatcatctctacaatgtagacaacattatattttatagaatttgtttgaaattgaggaagcagttaaattgtgcgctgtattttgcagattatggggattcaaattctagtaataggcttttttatttttatttttatacccttaaccagtttaattttttttttcctcattgttggggatgatgagaagaaatgatttgggaaaattaagtaacaacgacctagaaaagtgagaacaatctcatttaccatcatgtatccagtagtggataattcattttgatggcttctatttttggccaaatgagaattaagccagtgcctgagactgtcagaagttgacctttgcactggcattaaagagtcatagaaaaagaatcatggatatttatgaattaaggtaagaggtgtggcttttttttttttcttttttccagccgttgaccaattatagttcggctgttgactgagaagtttgtggtgggaaaacgtttgccatattttctttgcatttgaataattgtcttgtacttagaaaaaaggcgtctatgaatgaccagtgtttttggtcgccaaatgttgctgacaaacttatcccaaaactttagtggcttaaaaaaacctgcccccaactgttagtcaatctgagctgggctcagctgggctgttcttctgccagcctgcaggtggccactcatgtggtcagcaggtcggcggagagactgggatggctgggcttctctctctgcctgcagtcctgagtctctccttcttcgtgtagtctctttcagtggcctggctggcagggtagctagacctctcacatgcagctcagagctcccaagagctcaaaagcagaaatggccaggccttctgaaaacttaagtccagaattgtcacagtgtcccttctacttccctctattgatgatgatgatgatgatgatgatgatgatgatgatgatgatgatggttttttctaatcagaagaaagctggggtatgccctctacttactaaacaagtcacaagcccagctcagattcaagaaaagggtgtgaagtagaggtgcagttaagtggggggccactagtctaacagacggtcacaaccagtgccatggaaaaccaaggatattagcaaaagcagaagttgctagtgaccttgggaagccgaagctgcttacagtagctgggacaagctgaaagtcagactaagaaataaagagagggccttcaagaagcttcctgaatgatttctgctagccctgagcctatttttggaaccagcacttggggaaactgatcttgtgaggatggatgtgtttagggacacagggcttttgagagcagcaccaccccactggggcatccccagacttgggaaacgtgactctttcttaatgccactgggttttagtcaggccacagtgagaaggaacagccctaacaggcctccagccaggttgaatgagctcatttttgttgtagccaaccagtaagatttgctaatgttctacattaagtgccttctccaaagacatccctctttgcctcatatgttgaatcatccagtgcggatatttcaatgaaaatatcattggttgacttttgtgatggtaataatgctatggcatctttgccatgaagttgtggcctccttggattcttctgactttggcttctgaaaggaaggcctagatccagccctggtggtagttcctttctgaggtctctcagtcccttgagactttggggtagtttggctgccattctcactgacaaaatgtatatcagcccccacctccaccccccaatattccttgaactttgaattgcttcagaacacaggtgtggcctgaaggtattcccttattagggaagtgtcactgctgtcttctagtcaaacttgtaaagaaaaagattccagttcagtatttgcagcaagaagcttgaatgctgttctttttatcgcattgttacatcgactcattctccattttgctttggttttgtcttgacttgacttgactttgggggtaaagtctttcaccagcacacaagagtttgattgtacaaatatatcttctgcattaacatctctgcctgttgcttaagatcagttgcttttatactcagaatggaaatacctgatcttggctagttttgttataagatattgatttcatttagatttccctccacgaggtcagcaaactatcatgttcttatgtaaacttaggccaaggccagagttatcatagtccctaggttgctacggcttatcatgtgcttggtaaaaggtgatcgcaggttctcagacgagtttactttacatgagatggaatcaggcagagaggctgggatgatggagaaagctcgaggtgaagttttaaaaaaaaagttgtggaaaggaaagttccaaagaggtggtttctgaggaagtcagagcgcccagggccagagcagtcagtaatgggtgaatgaggttgtttggaaagtcggtgtgacagacacatggatgccatctacttctaggttgctggtgggtattaaatatgcacaatattccatagctcactgaggattttaaaattataagcataggattttatattttggggtgaaagaattatctggcacattaggtattggagtttaaaaaaaaagccaaatttcacagtcttaataactttttttaaaaaaaactaaaaggcgcttcatgtccagtgtgtggcccttctgaaacttatggtcatctctcccactgaaaccaaggtcttttcaaatgtggctaaatggggatgaggagacacgggtaggactttcttggtgtgtgtgcattctttaaagagccaagttgcttcggggaaacagccaggaaaatggtcaagattatttttagaggttattttattggggattttaagaactaataacatcttgagttatttttaattcagggggatgtggaaaggtttgcaattgtcaagtgttttgttgtagcttagtatccataagggaaacttagactatagacataactacaaagccagtgcagcttttgttttctgtatgttgttgggggatcaactttcacacatagcaagcacatggcctccctgatgtcaggatgcctttgttaggatctgtatttgcccttaattttgttgaaatcttttttccttcttcctcttgaaaagttccaaaatatagtttattgtatctttcatcactaaaaatttgttcctttttcactatgggcagttcacacaaggcaaaaactattgaacagttggttttagtgtgttgtataactttgctgtatatcaaactaattttgacaagttttcatcctaagcctcaaatcatgtaattaataatttgcctgtttatttatgacctaattgtgattcttttattaataaaagctaatgggaaaaggatccctgattaagctgatgactagacctacaattaattttcctgcagtatatgaagtattgtaccagagtattaaaagatatgtaatattttattgataaatctatcctttaaaaggaatacgttttaggatgtcatcattttgatgtgaatcatgtaaatgttgataatatgctgtttattatacatttagtgtttcaagagattcacttaattgcctttttgcccacgtatattatgtagtctatttgcaactgttcttaaaaaaatgacattaaaagaatagtttatgtagagaaacattagtggatgttaattgtctccccacctatatttatgggtgttagcgcaactgctttgctagttgcaaagctgtattatcagagtaaaagtgtatttgtaaactgtatgggaactaaaaattaggaataaaaccattttcttatatgatggca

SEQ ID NO: 22 - DNA, 4039 bp, Homo sapiens
ctctcctcgcggcgcgagtttcaggcagcgctgcgtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactgatggccacccgcccacagccaggccgagagcagacaccagcagccctgtcacgccgggctctacgtcccagggagggaggggcggcccacacccaggcccgcaccgctgggagtctgaggcctgagtgagtgtttggccgaggcctgcatgtccggctgaaggctgagtgtccggctgaggcctgagcgagtgtccagccaagggctgagtgtccagcacacctgccgtcttcacttccccacaggctggcgctcggctccaccccagggccagcttttcctcaccaggagcccggcttccactccccacataggaatagtccatccccagattcgccattgttcacccctcgccctgccctcctttgccttccacccccaccatccaggtggagaccctgagaaggaccctgggagctctgggaatttggagtgaccaaaggtgtgccctgtacacaggcgaggaccctgcacctggatgggggtccctgtgggtcaaattggggggaggtgctgtgggagtaaaatactgaatatatgagtttttcagttttgaaaaaaa

SEQ ID NO: 23 - DNA, 6240 bp, Homo sapiens
agatgcgatccagcggctctgggggcggcagcggtggtagcagctggtacctcccgccgcctctgttcggagggtcgcggggcaccgaggtgctttccggccgccctctggtcggccacccaaagccgcgggcgctgatgatgggtgaggagggggcggcaagatttcgggcgcccctgccctgaacgccctcagctgctgccgccggggccgctccagtgcctgcgaactctgaggagccgaggcgccggtgagagcaaggacgctgcaaacttgcgcagcgcgggggctgggattcacgcccagaagttcagcaggcagacagtccgaagccttcccgcagcggagagatagcttgagggtgcgcaagacggcagcctccgccctcggttcccgcccagaccgggcagaagagcttggaggagccaaaaggaacgcaaaaggcggccaggacagcgtgcagcagctgggagccgccgttctcagccttaaaagttgcagagattggaggctgccccgagaggggacagaccccagctccgactgcggggggcaggagaggacggtacccaactgccacctcccttcaaccatagtagttcctctgtaccgagcgcagcgagctacagacgggggcgcggcactcggcgcggagagcgggaggctcaaggtcccagccagtgagcccagtgtgcttgagtgtctctggactcgcccctgagcttccaggtctgtttcatttagactcctgctcgcctccgtgcagttgggggaaagcaagagacttgcgcgcacgcacagtcctctggagatcaggtggaaggagccgctgggtaccaaggactgttcagagcctcttcccatctcggggagagcgaagggtgaggctgggcccggagagcagtgtaaacggcctcctccggcgggatgggagccatcgggctcctgtggctcctgccgctgctgctttccacggcagctgtgggctccgggatggggaccggccagcgcgcgggctccccagctgcggggccgccgctgcagccccgggagccactcagctactcgcgcctgcagaggaagagtctggcagttgacttcgtggtgccctcgctcttccgtgtctacgcccgggacctactgctgccaccatcctcctcggagctgaaggctggcaggcccgaggcccgcggctcgctagctctggactgcgccccgctgctcaggttgctggggccggcgccgggggtctcctggaccgccggttcaccagccccggcagaggcccggacgctgtccagggtgctgaagggcggctccgtgcgcaagctccggcgtgccaagcagttggtgctggagctgggcgaggaggcgatcttggagggttgcgtcgggccccccggggaggcggctgtggggctgctccagttcaatctcagcgagctgttcagttggtggattcgccaaggcgaagggcgactgaggatccgcctgatgcccgagaagaaggcgtcggaagtgggcagagagggaaggctgtccgcggcaattcgcgcctcccagccccgccttctcttccagatcttcgggactggtcatagctccttggaatcaccaacaaacatgccttctccttctcctgattattttacatggaatctcacctggataatgaaagactccttccctttcctgtctcatcgcagccgatatggtctggagtgcagctttgacttcccctgtgagctggagtattcccctccactgcatgacctcaggaaccagagctggtcctggcgccgcatcccctccgaggaggcctcccagatggacttgctggatgggcctggggcagagcgttctaaggagatgcccagaggctcctttctccttctcaacacctcagctgactccaagcacaccatcctgagtccgtggatgaggagcagcagtgagcactgcacactggccgtctcggtgcacaggcacctgcagccctctggaaggtacattgcccagctgctgccccacaacgaggctgcaagagagatcctcctgatgcccactccagggaagcatggttggacagtgctccagggaagaatcgggcgtccagacaacccatttcgagtggccctggaatacatctccagtggaaaccgcagcttgtctgcagtggacttctttgccctgaagaactgcagtgaaggaacatccccaggctccaagatggccctgcagagctccttcacttgttggaatgggacagtcctccagcttgggcaggcctgtgacttccaccaggactgtgcccagggagaagatgagagccagatgtgccggaaactgcctgtgggtttttactgcaactttgaagatggcttctgtggctggacccaaggcacactgtcaccccacactcctcaatggcaggtcaggaccctaaaggatgcccggttccaggaccaccaagaccatgctctattgctcagtaccactgatgtccccgcttctgaaagtgctacagtgaccagtgctacgtttcctgcaccgatcaagagctctccatgtgagctccgaatgtcctggctcattcgtggagtcttgaggggaaacgtgtccttggtgctagtggagaacaaaaccgggaaggagcaaggcaggatggtctggcatgtcgccgcctatgaaggcttgagcctgtggcagtggatggtgttgcctctcctcgatgtgtctgacaggttctggctgcagatggtcgcatggtggggacaaggatccagagccatcgtggcttttgacaatatctccatcagcctggactgctacctcaccattagcggagaggacaagatcctgcagaatacagcacccaaatcaagaaacctgtttgagagaaacccaaacaaggagctgaaacccggggaaaattcaccaagacagacccccatctttgaccctacagttcattggctgttcaccacatgtggggccagcgggccccatggccccacccaggcacagtgcaacaacgcctaccagaactccaacctgagcgtggaggtggggagcgagggccccctgaaaggcatccagatctggaaggtgccagccaccgacacctacagcatctcgggctacggagctgctggcgggaaaggcgggaagaacaccatgatgcggtcccacggcgtgtctgtgctgggcatcttcaacctggagaaggatgacatgctgtacatcctggttgggcagcagggagaggacgcctgccccagtacaaaccagttaatccagaaagtctgcattggagagaacaatgtgatagaagaagaaatccgtgtgaacagaagcgtgcatgagtgggcaggaggcggaggaggagggggtggagccacctacgtatttaagatgaaggatggagtgccggtgcccctgatcattgcagccggaggtggtggcagggcctacggggccaagacagacacgttccacccagagagactggagaataactcctcggttctagggctaaacggcaattccggagccgcaggtggtggaggtggctggaatgataacacttccttgctctgggccggaaaatctttgcaggagggtgccaccggaggacattcctgcccccaggccatgaagaagtgggggtgggagacaagagggggtttcggagggggtggaggggggtgctcctcaggtggaggaggcggaggatatataggcggcaatgcagcctcaaacaatgaccccgaaatggatggggaagatggggtttccttcatcagtccactgggcatcctgtacaccccagctttaaaagtgatggaaggccacggggaagtgaatattaagcattatctaaactgcagtcactgtgaggtagacgaatgtcacatggaccctgaaagccacaaggtcatctgcttctgtgaccacgggacggtgctggctgaggatggcgtctcctgcattgtgtcacccaccccggagccacacctgccactctcgctgatcctctctgtggtgacctctgccctcgtggccgccctggtcctggctttctccggcatcatgattgtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagagatctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtggaggagagagaggcaatggctccttcacaaaccagagaccaaatgtcacgttttgttttgtgccaacctattttgaagtaccaccaaaaaagctgtattttgaaaatgctttagaaaggttttgagcatgggttcatcctattctttcgaaagaagaaaatatcataaaaatgagtgataaatacaaggcccagatgtggttgcataaggtttttatgcatgtttgttgtatacttccttatgcttctttcaaattgtgtgtgctctgcttcaatgtagtcagaattagctgcttctatgtttcatagttggggtcatagatgtttccttgccttgttgatgtggacatgagccatttgaggggagagggaacggaaataaaggagttatttgtaatgactaa

SEQ ID NO: 24 - DNA, 20635 bp, Homo sapiens
gccgctccgctccaacacaaaatagggccgcctcttctcctttctccccctctcgagtggggtgccccgggcaaaaggcccccccggatctagcgccgaaggcttcacgaatcttcacgaccgctgcccagctcttgggccaggaaatagccccttcgcaggaaccaccctaccggccgaacaggaggcggagggggggaggcggagcggcgccgcgctgcactactttcctctccggttgcaaatggctgcctcgttccccactttccgctcagtttcctgaccccccggtgccgggagccggggttgggccatgcacctctaggccgcctgcgatcacagtcagccgggggtcgagggggtgccaccgaccagagccggccaggccgggggcggggcagctcccgaggccagaggggaagggaggcgagcgcagggcctggagcggccggaggggagcgggcagagggctcgcaccgcccgccccttccttttcctcgcctacctagcctcctcccttccccggggggagcagaaggtggggggctcgaagccgccgagggtgagcgctcggggtcgagaagcccggcgctgggtgtgtgtcaggttcagccccgcggccccgccggccccgcgtcgccgtagctcgcgcggccccgggcgccggccggggcggggagaggggctcggcgctcctgcgagggtctcacgttccatccgggccaggcgcggggcggcgcggcattccttccgggctgctggggaggcgcctcgacgttccatctggagagcctcgacgttccgcccgagcccggcgcgggcggccggggcgctggccgggccctaggactgagaggccgcccggcgacgcggatgcggagcctgctcgcccaagatcaaagccaccggtgctctctttgtgtccgctcgggattcgccgccctggggctgtccatggaaacctaaactgctggaacctgaggcagagaacccctctttggcttcttgctgtttttttgtggggggagggtggccactccgacctggatttaccgttcttggcccccctaagcccccccgtgcggggggcggctgtgatcgctctggcggttggaggtcggggagcggcccgggctctggccatgttctcggatgaggatttctggatcgccctgtgaagaggtctccccgagagggccctgcccagtcggagagagggatggacagccagaagctggctggtgaggataaagattcagaaccggcagctgatggacctgcagcttctgaggacccaagtgccactgagtcagacctgcccaacccacatgtgggagaggtctctgtccttagttctgggagtcccaggcttcaggagactcctcaggactgcagtgggggtccggtgcggcgttgtgctctctgtaactgcggggagcccagtctacacgggcagcgggagctacggcgctttgagttgccatttgattggccccggtgtccagtggtgtcccctggggggagcccagggcccaatgaggcagtgctgcccagtgaggacctatcacagattggtttccctgagggccttacacctgcccacctaggagaacctggagggtcctgctgggctcaccattggtgtgctgcatggtcggcaggcgtatgggggcaggagggcccagaactatgtggtgtggacaaggccatcttctcagggatctcacagcgctgctcccactgcaccaggctcggtgcctccatcccttgccgctcacctggatgtccacggctttaccacttcccctgcgcgactgccagcggttccttcctatccatgaaaacactgcagctgctatgcccagagcacagtgagggggctgcatatctggaggaggctcgctgtgcagtgtgtgaggggccaggggagttgtgtgacctgttcttctgtaccagctgtgggcatcactatcacggggcctgcctggacactgctctgactgcccgcaaacgtgctggctggcagtgccctgaatgcaaagtgtgccaagcctgcaggaaacctgggaatgactctaagatgttggtttgtgagacgtgtgacaaaggataccatactttctgcctaaaaccacccatggaggaactgcctgctcactcttggaagtgcaaggcgtgccgggtgtgccgggcctgtggggcgggctcagcagaactgaatcccaactcggagtggtttgagaactactctctctgtcaccgctgtcacaaagcccagggaggtcagactatccgctccgttgctgagcagcataccccggtgtgtagcagattttcacccccagagcctggcgatacccccactgacgagcccgatgctctgtacgttgcatgccaagggcagccaaagggtgggcacgtgacctctatgcaacccaaggaaccagggcccctgcaatgtgaagccaaaccactagggaaagcaggggtccaacttgagccccagttggaggcccccctaaacgaggagatgccactgctgcccccacctgaggagtcacccctgtccccaccacctgaggaatcacccacgtccccaccacctgaggcatcacgcctgtcaccaccacctgaggaattgcccgcatccccacttcctgaggcattgcacctgtcccggccgctggaggaatcgcccctctctccgccgcctgaggagtctcctctgtctcccccacctgaatcatcacctttttctccactggaggagtcgcccttgtctccaccggaagagtcacccccatctcctgcacttgagacgcctctatccccaccacctgaagcatcgcccctgtccccaccatttgaagaatctcctttgtccccgccacctgaggaattgcccacttccccgccacctgaagcatctcgcctgtctccaccacctgaggagtcacccatgtcccctccacctgaagagtcacccatgtctccaccaccggaggcatctcgtctgttcccaccatttgaagagtctcctctgtcccctccacctgaggagtctcccctttccccaccacctgaggcatcacgcctgtccccaccacctgaggactcgcctatgtccccaccacctgaagaatcacctatgtcccccccacctgaggtatcgcgcctatcccccctgcctgtggtgtcacgcctgtctccaccgcctgaggaatctcccttgtccccaccgcctgaggagtctcccacgtcccctccacctgaggcttcacgcctctccccaccacctgaggactcccccacatccccaccacctgaggactcacctgcttccccaccaccggaggactcgctcatgtccctgccgctggaggagtcacccctgttgccactacctgaggagccgcaactctgcccccggtccgaggggccgcacctgtcaccccggcctgaggagccgcacctgtccccccggcctgaggagccacacctatctccgcaggctgaggagccacacctgtccccccagcctgaggagccatgcctatgcgctgtgcctgaggagccacacttgtccccccaggctgagggaccacatctgtcccctcagcctgaggaattgcacctgtccccccagactgaggagccgcacctgtctcctgtgcctgaggagccatgcttgtccccccaacctgaggaatcacacctgtccccccagtctgaggagccatgcctgtccccccggcctgaggaatcgcatctgtcccctgagcttgagaagccacccctgtcccctcggcctgaaaagccccctgaggagccaggccaatgccctgcacctgaggagctgcccttgttccctccccctggggaaccatccttatctcccttgcttggagagccagccctgtctgagcctggggaaccacctctgtcccctctgcccgaggagctgccgttgtccccatctggggagccatccttgtcgcctcagctgatgccaccagatccccttcctcctccactctcacccatcatcacagctgcggccccaccggccctgtctcctttgggggagttagagtacccctttggtgccaaaggggacagtgaccctgagtcaccgttggctgcccccatcctggagacacccatcagccctccaccagaagctaactgcactgaccctgagcctgtcccccctatgatccttcccccatctccaggctccccagtggggccggcttctcccatcctgatggagccccttcctcctcagtgttcgccactccttcagcattccctggttccccaaaactcccctccttcccagtgctctcctcctgccctaccactgtccgttccctccccgttgagtcccatagggaaggtagtgggggtctcagatgaggctgagctgcacgagatggagactgagaaagtttcagaacctgaatgcccagccttggaacccagtgccaccagtcctctcccttccccaatgggggacctttcctgccccgcccccagccctgccccagccctggatgacttctctggcctaggggaagacacagcccctctggatgggattgatgctccgggttcacagccagagcctggacagacccctggcagtttggctagtgaacttaaaggctcccctgtgctcctggaccccgaggagctggcccctgtgacccctatggaggtctaccccgaatgcaagcagacagcagggcagggctcaccatgtgaagaacaggaagagccacgtgcaccggtggcccccacaccacccactctcatcaaatccgacatcgttaacgagatctctaatctgagccagggtgatgccagtgccagttttcctggctcagagcccctcctgggctctccagacccggaggggggtggctccctgtccatggagttgggggtctctacggatgttagtccagcccgagatgagggctccctacggctctgtactgactcactgccagagactgatgactcactattgtgcgatgctgggacagctatcagcggaggcaaagctgagggggagaaggggcggcggcgcagctccccagcccgttcccgcatcaaacagggtcgcagcagcagtttcccaggaagacgccggcctcgtggaggagcccatggaggacgtggtagaggacgggcccggctaaagtcaactgcttcttccattgagactctggtagttgctgacattgatagctctcccagtaaggaggaggaggaagaagatgatgacaccatgcagaataccgtggttctcttctccaacacagacaaatttgtcctaatgcaggacatgtgtgtggtatgtggcagctttggccggggggcagagggccacctccttgcctgttcgcagtgctctcagtgctatcacccttactgtgtcaacagcaagatcaccaaggtgatgctgctcaagggctggcgttgtgtggagtgtattgtgtgtgaggtgtgtggccaggcctccgacccctcacgcctgctgctctgtgatgactgtgatattagctaccacacatactgcctggaccccccactgctcaccgtccccaagggcggctggaagtgcaagtggtgtgtgtcctgtatgcagtgtggggctgcttcccctggcttccactgtgaatggcagaatagttacacacactgtgggccctgtgccagcctggtgacctgccctatctgtcatgctccttacgtagaagaggacctactaatccagtgccgccactgtgaacggtggatgcatgcaggctgtgagagcctcttcacagaggacgatgtggagcaggcagccgatgaaggctttgactgtgtctcctgccagccctacgtggtaaagcctgtggcgcctgttgcacctccagagctggtgcccatgaaggtgaaagagccagagccccagtactttcgcttcgaaggtgtgtggctgacagaaactggcatggccttgctgcgtaacctgaccatgtcaccactgcacaagcggcgccaacggcgaggacggcttggcctcccaggcgaggcaggattggagggttctgagccctcagatgcccttggccctgatgacaagaaggatggggacctggacaccgatgagctgctcaagggtgaaggtggtgtggagcacatggagtgcgaaattaaactggagggccccgtcagccctgatgtggagcctggcaaagaggagaccgaggaaagcaaaaaacgcaagcgtaaaccatatcggcctggcattggtggtttcatggtgcgacagcggaaatcccacacacgcacgaaaaaggggcctgctgcacaggcggaggtgttgagtggggatgggcagcccgacgaggtgatacctgctgacctgcctgcagagggcgccgtggagcagagcttagctgaaggggatgagaagaagaagcaacagcggcgagggcgcaagaagagcaaactggaggacatgttccctgcttacttgcaggaagccttctttgggaaggagctgctggacctgagccgtaaggccctttttgcagttggggtgggccggccaagctttggactagggaccccaaaagccaagggagatggaggctcagaaaggaaggaactccccacatcgcagaaaggagatgatggtccagatattgcagatgaagaatcccgtggcctcgagggcaaagccgatacaccaggacctgaggatgggggcgtgaaggcatccccagtgcccagtgaccctgagaagccaggcaccccaggtgaagggatgcttagctctgacttagacaggatttccacagaagaactgcccaagatggaatccaaggacctgcagcagctcttcaaggatgttctgggctctgaacgagaacagcatctgggttgtggaacccctggcctagaaggcagccgtacgccactgcagaggccctttcttcaaggtggactccctttgggcaatctgccctccagcagcccaatggactcctacccaggcctctgccagtccccgttcctggattctagggagcgcgggggcttctttagcccggaacccggtgagcccgacagcccctggacgggctcaggtggcaccacgccctccacccccacaacccccaccacggagggtgagggcgacggactctcctataaccagcggagtcttcagcgctgggagaaggatgaggagttgggccagctgtccaccatctcacctgtgctctatgccaacattaattttcctaatctcaagcaagactacccagactggtcaagccgttgcaaacaaatcatgaagctctggagaaaggttccagcagctgacaaagccccctacctgcaaaaggccaaagataaccgggcagctcaccgcatcaacaaggtgcagaagcaggctgagagccagatcaacaagcagaccaaggtgggcgacatagcccgtaagactgaccgaccggccctacatctccgcattcccccgcagccaggggcactgggcagcccgccccccgctgctgcccccaccattttcattggcagccccactacccccgccggcttgtctacctctgcggacgggttcctgaagccgccggcgggctcggtgcctggccctgactcgcctggtgagctcttcctcaagctcccaccccaggtgcccgcccaagtgccttcgcaggacccctttggactggcccctgcctatcccctggagccccgcttccccacggcaccgcccacctatcccccctatcctagtcctacgggggcccctgcgcagcccccgatgctgggcgcctcatctcgtcctggggctggccagccaggggaattccacactaccccacctggcacccccagacaccagccctccacacctgacccattcctcaaaccccgctgcccctcgctggataacttggctgtgcctgagagccctggggtagggggaggcaaagcttccgagcccctgctctcgcccccaccttttggggagtcccggaaggccctagaggtgaagaaggaagagcttggggcatcctctcctagctatgggcccccaaacctgggctttgttgactcaccctcctcaggcacccacctgggtggcctggagttaaagacacctgatgtcttcaaagcccccctgacccctcgggcatctcaggtagagccccagagcccgggcttgggcctaaggccccaggagccaccccctgcccaggctttggcaccttctcctccaagtcacccagacatctttcgccctggctcctacactgacccatatgctcagcccccattgactcctcggccccaacctccgccccctgagagctgctgtgctctgccccctcgctcactgccctccgaccctttctcccgagtgcctgccagtcctcagtcccagtccagctcccagtctccactgacaccccggcctctgtctgctgaagctttttgcccatcacccgttacccctcgcttccagtcccctgacccttattctcgcccaccctcacgccctcagtcccgtgacccatttgccccattgcataagccaccccgaccccagccccctgaagttgcctttaaggctgggtctctagcccacacttcgctgggggctggggggttcccagcagccctgcccgcggggccagcaggtgagctccatgccaaggtcccaagtgggcagccccccaattttgtccggtcccctgggacgggtgcatttgtgggcaccccctctcccatgcgtttcactttccctcaggcagtaggggagccttccctaaagccccctgtccctcagcctggtctcccgccaccccatgggatcaacagccattttgggcccggccccaccttgggcaagcctcaaagcacaaactacacagtagccacagggaacttccacccatcgggcagccccctggggcccagcagcgggtccacaggggagagctatgggctgtccccactacgccctccgtcggttctgccaccacctgcacccgacggatccctcccctacctgtcccatggagcctcacagcgatcaggcatcacctctcctgtcgaaaagcgagaagacccagggactggaatgggtagctctttggcgacagctgaactcccaggtacccaggacccaggcatgtccggccttagccaaacagagctggagaagcaacggcagcgccagcgactacgagagctgctgattcggcagcagatccagcgcaacaccctgcggcaggagaaggaaacagctgcagcagctgcaggagcagtggggcctccaggcagctggggtgctgagcccagcagccctgcctttgagcagctgagtcgaggccagaccccctttgctgggacacaggacaagagcagccttgtggggttgcccccaagcaagctgagtggccccatcctggggccagggtccttccctagcgatgaccgactctcccggccacctccaccagccacgccttcctctatggatgtgaacagccggcaactggtaggaggctcccaagctttctatcagcgagcaccctatcctgggtccctgcccttacagcagcaacagcaacaactgtggcagcaacaacaggcaacagcagcaacctccatgcgatttgccatgtcagctcgctttccatcaactcctggacctgaacttggccgccaagccctaggttccccgttggcgggaatttccacccgtctgccaggccctggtgagccagtgcctggtccagctggtcctgcccagttcattgagctgcggcacaatgtacagaaaggactgggacctgggggcactccgtttcctggtcagggcccacctcagagaccccgtttttaccctgtaagtgaggacccccaccgactggctcctgaagggcttcggggcctggcggtatcaggtcttcccccacagaaaccctcagccccaccggcccctgaattgaacaacagtcttcatccaacaccccacaccaagggtcctaccctgccaactggtttggagctggtcaaccggcccccgtcgagcactgagcttggccgccccaatcctctggccctggaagctgggaagttgccctgtgaggatcccgagctggatgacgattttgatgcccacaaggccctagaggatgatgaagagcttgctcacctgggtctgggtgtggatgtggccaagggtgatgatgaacttggcaccttagaaaacctggagaccaatgacccccacttggatgacctgctcaatggagacgagtttgacctgctggcatatactgatcctgagctggacactggggacaagaaggatatcttcaatgagcacctgaggctggtagaatcggctaatgagaaggctgaacgggaggccctgctgcggggggtggagccaggacccttgggccctgaggagcgccctccccctgctgctgatgcctctgaaccccgcctggcatctgtgctccctgaggtgaagcccaaggtggaggagggtggacgccacccttctccttgccaattcaccattgctacccccaaggtagagcccgcacctgctgccaattcccttggcctggggctaaagccaggacagagcatgatgggcagccgggatacccggatgggcacagggccattttctagcagtgggcacacagctgagaaggcctcctttggggccacgggaggaccaccagctcacctgctgacccccagcccactgagtggcccaggaggatcctccctgctggaaaagtttgagctcgagagtggggctttgaccttgcctggtggacctgcagcatctggggatgagctagacaagatggagagctcactggtagccagcgagttacccctgctcattgaggacctgttggagcatgagaagaaggagctgcagaagaagcagcagctttcagcacagttgcagcctgcccagcagcagcagcaacagcagcagcagcattccctactgtctgcaccaggccctgcccaggccatgtctttgccacatgagggctcttctcccagtttggctgggtcccaacagcagctttccctgggtcttgcaggtgcccgacagccaggtttgccccagccactgatgcccacccagccaccagctcatgccctccagcaacgcctggctccatccatggctatggtgtccaatcaagggcatatgctaagtgggcagcatggagggcaggcaggcttggtaccccagcagagctcacagccagtgctatcacagaagcccatgggcaccatgccaccttccatgtgcatgaagccgcagcaattggcaatgcagcagcagctggcaaacagcttcttcccagatacagacctggacaaatttgctgcagaagatatcattgatcccattgcaaaggccaagatggtggctttgaaaggcatcaagaaagtgatggctcagggcagcattggggtggcacctggtatgaacagacagcaagtgtctctgctagcccagaggctctcggggggacctagcagtgatctgcagaaccatgtggcagctgggagtggccaggagcggagtgctggtgatccctcccagcctcgtcccaacccgcccacttttgctcagggagtgatcaatgaagctgaccagcggcagtatgaggagtggctgttccatacccagcagctcctacagatgcagctgaaggtgctagaggagcagattggtgtacaccgcaagtcccggaaggctctgtgtgccaagcagcgcactgccaaaaaagctggccgtgagttcccagaagctgatgctgagaagctcaagctggttacagagcagcagagcaagatccagaaacaactggatcaggtccggaaacagcagaaggagcacactaatctcatggcagaatatcggaacaagcagcagcaacaacagcagcagcagcagcaacaacagcaacagcactcagctgtgctggctctcagcccttcccagagtccccggctgctcaccaagctccctggtcagctgctccctggccatgggctgcagccaccacaggggcctccgggtgggcaagccggaggtcttcgcctgacccctgggggtatggcactacctggacagcctggtggccccttccttaatacagctctggcccaacagcagcaacagcaacattctggtggggctggatccctggctggcccttcagggggcttcttccctggcaaccttgctcttcgaagcctcggacctgattcaaggcttttacaggaaaggcagctgcagctgcagcagcaacgtatgcagctggcccagaaactgcagcagcagcagcagcagcaacagcagcagcagcaccttctaggacaggtggcaatccagcagcaacagcagcagggtcctggagtacagacaaaccaagctctgggtcccaagccccagggccttatgcctcccagcagccaccaaggcctcctggtccagcagctgtcccctcaaccaccccaggggccccagggcatgctgggccctgcccaggtggctgtgttgcagcagcagcaccctggagctttgggcccccagggccctcacagacaggtgcttatgacccagtcccgggtgctcagttccccccagctggcacagcagggtcagggccttatgggacacaggctggtcacagcccagcagcagcagcagcaacaacagcaccaacagcaagggtccatggcagggctgtcccatcttcagcagagtctgatgtcacacagtgggcagcccaaactgagcgctcagcccatgggctctttacagcagcttcagcagcagcagcagctgcaacagcaacagcaacttcagcagcagcagcagcagcagctacaacagcaacagcaacttcagcagcaacagcttcaacagcagcaacagcagcagcagcttcaacaacagcagcagcaacagcttcaacagcagcaacagcagctacaacagcaacagcaacaacaacagcagcagtttcaacagcagcagcaacagcagcagatgggccttttaaaccagagtcgaactttactgtctcctcagcaacaacagcagcagcaagtggcacttggccctggcatgccagcaaagcctcttcaacacttttctagccctggagccctgggtccaaccctcctcctgacgggcaaggaacaaaacaccgtagacccagccgtttcttcagaggccactgaggggccctctacacatcagggagggccgttagcaataggaactacccctgagtcaatggccactgaaccaggagaggtaaagccctcactctctggggactcacaactcctgcttgtccaaccccagccccagcctcagcccagctctctgcagctgcagccacctctgaggcttccaggacaacagcagcagcaagttagcctgctccacacagcaggtggaggaagccatgggcagctaggcagtggatcatcttctgaggcctcatctgtgccccacctgctggctcagccctctgtttccttaggggatcagcctgggtccatgacccagaaccttctgggcccccaacagcccatgctagagcggcccatgcaaaataatacagggccacaacctcccaaaccaggacctgtcctccagtctgggcagggtctgcctggggttggaatcatgcctacggtgggtcagcttcgagcacagctccaaggagtcctggccaaaaacccacagctgcggcacttaagtcctcagcagcagcagcagctacaggcactcctcatgcagcggcagctgcagcagagtcaggcagtacgccagaccccaccctaccaggagcctgggacccagacctctcccctccagggcctcctgggctgccaacctcaacttgggggcttccctggaccacagacaggccccctccaggagctaggggcagggcctcgacctcagggcccaccccggctccctgccccaccaggagccttatctacaggaccagtccttggccctgtccatcccacacctccaccatccagccctcaagagccaaagagaccttcacaattaccttcccccagctcccagcttcccactgaggcccagctccctcccacccatccagggacccccaaacctcaggggccaaccttggagccgcctcctgggagggtctcacctgctgctgcccagcttgcagataccttgtttagcaagggtctgggaccttgggatcccccagacaacctagcagaaacccagaagccagagcagagcagcctggtacctgggcatctggaccaggtgaatggacaggtggtgcctgaggcatcccaactcagcatcaagcaggaacctcgggaagagccatgtgccctgggagcccagtcagtgaagagggaggccaatggggagccaataggggcaccaggaaccagcaaccacctcctgctggcaggccctcgctcagaagctgggcatctgctcttgcagaagctactccgggcaaagaatgtgcaactcagcactgggcgggggtccgaggggctgcgagctgagatcaacgggcacattgacagcaagctggctgggctggagcagaaactacagggtacccccagcaacaaggaggatgcagcagcaaggaagcctttgacaccgaagcccaagcgggtacagaaggcaagcgacaggttggtgagctcccgaaagaagctgcggaaggaggacggggtcagggccagcgaggccttgctgaaacagctgaaacaggagctgtccctgctgcccctaacggagcctgctatcaccgccaattttagcctctttgccccctttggcagtggctgcccagtcaatgggcagagccagctgaggggggcctttggaagtggggcgctgcccactggccctgactactattcccagctgcttaccaagaataacctgagtaacccgccgacaccaccctcgtcgctgccccccaccccacccccatcggtgcagcagaagatggtgaatggcgtcaccccatctgaagagctgggggagcaccccaaggatgctgcctctgcccgggatagtgaaagggcactgagggatacttcagaggtgaagagtctagacctgctggctgccttgcctacaccccctcacaatcagactgaggatgtcaggatggagagtgatgaggatagcgattctcctgacagcattgtgccagcttcatcccctgagagcatcttgggggaggaggcccctcgtttccctcatctgggctcaggccggtgggagcaagaggaccgggccctctcccctgtcatccccctcattcctcgggccagcatcccagtcttcccagataccaaaccttatggggcccttggcctggaggtccctggaaagctgcctgtcacaacttgggaaaagggcaaaggaagtgaggtgtcagtcatgctcacagtctctgctgctgcagccaagaacctgaatggcgtgatggtggcagtggcggagctgctgagcatgaagatccccaactcctatgaggtgctgttcccagagagccccgcccgggcaggcactgagccaaagaagggggaagctgagggtcctggtgggaaggaaaagggtctggaaggcaagagcccagacactggccctgattggctgaagcagtttgatgcagtgttgcctggctataccctgaagagccaactagacatcttgagcctcctgaaacaggagagccccgccccagagccacccactcagcacagctatacctacaatgtctccaatctggatgtgcgacagctctcggccccacctcctgaagaaccctccccgcccccttcccccttggcaccttctcctgccagtccccctactgagcccttggttgaacttcccaccgaacccttggctgagccacccgtcccctcacctctgccactggcctcatcccctgaatcagcccgacccaagccccgtgcccggccccctgaagaaggtgaagattcccgtcctcctcgcctcaagaaatggaaaggagtgcgctggaagcggcttcggctgctgctgaccatccagaagggcagtgggcggcaggaggatgagcgggaagtggcagagtttatggagcagcttggcacagccttgcgacctgacaaggtaccgcgagacatgcgtcgctgctgtttctgtcatgaggagggtgacggggccactgatgggcctgcccgtctgctgaacctggacctggacctgtgggtgcacctcaactgtgccctttggtccacggaggtgtatgagacccagggcggggcactgatgaatgtggaggttgccctgcaccgaggactgctaaccaagtgctccctgtgccagcgaactggtgccaccagcagctgcaatcgcatgcgttgccccaatgtctaccattttgcttgtgccatccgtgccaagtgcatgttcttcaaggacaagaccatgctgtgtccaatgcataagatcaaggggccctgtgagcaagagctgagctcttttgctgtcttccggcgggtctacattgagcgggacgaggtgaagcaaatcgctagcatcattcagcggggagaacggctgcacatgttccgtgtggggggccttgtgttccacgccatcggacagctgctgcctcaccagatggctgactttcatagtgccactgccctctatcccgtgggctacgaggccacgcgcatctattggagcctccgcaccaacaatcgtcgctgctgctatcgctgttctattggtgagaacaacgggcggccggagtttgtaatcaaagtcatcgagcagggcctggaggacctggtcttcactgacgcctctccccaggccgtgtggaatcgcatcattgagcctgtggctgccatgagaaaagaggctgacatgctgcgactcttccctgagtatctgaagggcgaggagctctttgggctgacggtgcatgccgtgcttcgcatagctgaatcactgcccggggtggagagctgtcaaaactatttattccgctatgggcgccacccccttatggagctgccactcatgatcaaccccactggctgtgcccgatcagagcctaaaatcctcacacactacaaacggccccataccctgaacagcaccagcatgtctaaggcatatcagagcaccttcacaggcgagaccaacaccccctacagcaagcagtttgtgcactccaagtcatctcagtaccggcggctgcgcaccgaatggaagaacaacgtgtacctggctcgctcccgtatccagggcctggggctctatgcagccaaggacctagaaaagcacacaatggttatcgagtacattggcaccatcattcggaacgaggtggccaaccggcgggagaaaatctacgaagagcagaatcgaggcatctacatgttccgaataaacaatgaacatgtgattgatgctacgttgaccggcggccctgccaggtacattaaccattcctgtgcccctaactgtgtggccgaagtcgtgacatttgacaaagaggacaaaatcatcatcatctccagccggcgaatccccaaaggagaggagctaacctatgactatcagtttgattttgaggacgatcagcacaagatcccctgccactgtggagcctggaattgtcggaaatggatgaactaagaagctttgaggctaccaggcaggggagtccccctacccacaacctcttccctgaaagggatgagggggaagagaggtagcagccagagccaggacccagggttggggctgccggctgacccggagcccctggagcaggaggctggggcagagggccctaggccaagcccaccctgggcaccagggacaatcctcttccccaccaccggccctcaggctggcatctctgcccccagctccaggaggggccagacagaagcagccattgggcatctcaggtttgagggggatatgggccgggaactacccagaagcatctgggaggcagcagggtgggggaagaggatgtgtggccgggcctcacagccctgctgctcccactgacctctccggcccaactcacggctgcaaagagacttgactaagcttgacaatcccaaaggccgggtcccacacctggccctgcctgccgggtcctgcccccaccctcacccccatccccctccctcttgatctgtctctgtttccctcttttcctctgtgtttctgtctctctatgggttgtgtttccttgttttccactctgacaaatgcaacatgaacgggaaagaggcgcccagctgcctaggagggcaagctgggcaagccgggcaaggagaccccgcacccacacctacctcatttaagtgttggattttttgctgttttgaaatgtgagaccctctccaagccccctactgccccaaccctctcccccacctcactgccctcttctgagtgggtggaaggggggtaggaggaggaagaaaaacaacaacaaaaaatccatctttgtttttaattatgggcatgggatggtggttgaggcaaatgatgatgaagattggggatgactggcccctagttgctctaggacttccttctccatctggacatgggggcaggagggagctaaacctaggaccaggatatctccctcctgttttcccaacctcatcatgagcctgtttgccctccagcccctggacgggttgggtggggggtagggtgagggctatccctgagtggcatgcccatacctagtgaggcagggtgtggcccggagctcccactttccctcagtcaccaaactgctgctggtctggtgggaaggggtggtgatgtgggggtgggggagcttagtgtcagcgcggggagggtggggggtatttatctatttatacatgggattgtacatagtcttgtggggcatgggggagccggctggaggtgagaaccctcccctctccccccaccccccggggagagcaaatgtaaaactactaatttttgtgctttatatattctatataaatatatctattttctttttacaaaaccagtttataaatggtaggggggtgtggggcggacacatggagctccccttgtgggggggccccctccattacccgacctaccgcccttttcctcaccccccaccccactccccaccccctggctgtgactgctgtaagatgggggtatagaggctgggcaattcccaccccctgttgtatagttggactatgttataacgcacaaaagagagctgaccccagggggagccagagggtgatgggttccttgcctccctttccttcccctttctgcccaagcttgtgctgcagttgaacctcttcctgggggtgggagtaggtaaggggtgggtgaggccccaaacccctctctggtagggaaccgtggggatgaagatgaagcttatatgcagttctcttctaggggctgtgggcaaagggcattttgtaattaatattttcaagaatcagatgtctggagtgtaggggtgggcttggtggtggtggacgggcgggcctgctggagggggagcttggtcgctgttgtgattttaggtttgtttttgttttgttttgaatttggggggttgtggattgttgggggtagggagattttttttttttaaagctgcttcctcaactgtttcaagctgcaaatgtttaagagaataacagcccccactcccacaggaaccgctgtaattaaatcagacagtaggaagactgggctgctgccctcaaagccacagcccttggatgttccttttccgagagcagaaggtctaggctacagggagggggagattggctcccgtgagtcaggctgtgtttggggcttgggccctgggattgggaaaaggggatggggcagactttgtaagcatatgctaggtatccgatagtcctgtagaatttagtgaagaaaccttatacagtttttaatttttatataaactataactcagacccaagctacaaggttggaattttggttggttttttttttaagtaccctgcctgtataattgcatcagaatcccccaccccaccccccgcccccgtgtttgtattttgggttggtttacactcgcacatactcagttttcagttttcccctttacagtcttctcccctcacctccaggaccctccccctttttaaaaaataaatcgctgacaagtgtgaatcccgtgaagactttattttgtgttgtgtgtatcctgtacagcaaggttggtccttcgtaacaacggatgaaatggttcccttttttaaagcgccctctctccctccaccctcagcgcccctgtccttggcatgttttgtatcagcgatcattctgaactgtacatatttatgttgcgagaggcaaagggcaagttttggattttgcttcttccaagtttgtttttaaacgacaaataaaaaaagaacattttaaata

SEQ ID NO: 25 - protein, 887 aa, Homo sapiens
MGEAEKFHYIYSCDLDINVQLKIGSLEGKREQKSYKAVLEDPMLKFSGLYQETCSDLYVTCQVFAEGKPLALPVRTSYKAFSTRWNWNEWLKLPVKYPDLPRNAQVALTIWDVYGPGKAVPVGGTTVSLFGKYGMFRQGMHDLKVWPNVEADGSEPTKTPGRTSSTLSEDQMSRLAKLTKAHRQGHMVKVDWLDRLTFREIEMINESEKRSSNFMYLMVEFRCVKCDDKEYGIVYYEKDGDESSPILTSFELVKVPDPQMSMENLVESKHHKLARSLRSGPSDHDLKPNAATRDQLNIIVSYPPTKQLTYEEQDLVWKFRYYLTNQEKALTKFLKCVNWDLPQEAKQALELLGKWKPMDVEDSLELLSSHYTNPTVRRYAVARLRQADDEDLLMYLLQLVQALKYENFDDIKNGLEPTKKDSQSSVSENVSNSGINSAEIDSSQIITSPLPSVSSPPPASKTKEVPDGENLEQDLCTFLISRACKNSTLANYLYWYVIVECEDQDTQQRDPKTHEMYLNVMRRFSQALLKGDKSVRVMRSLLAAQQTFVDRLVHLMKAVQRESGNRKKKNERLQALLGDNEKMNLSDVELIPLPLEPQVKIRGIIPETATLFKSALMPAQLFFKTEDGGKYPVIFKHGDDLRQDQLILQIISLMDKLLRKENLDLKLTPYKVLATSTKHGFMQFIQSVPVAEVLDTEGSIQNFFRKYAPSENGPNGISAEVMDTYVKSCAGYCVITYILGVGDRHLDNLLLTKTGKLFHIDFGYILGRDPKPLPPPMKLNKEMVEGMGGTQSEQYQEFRKQCYTAFLHLRRYSNLILNLFSLMVDANIPDIALEPDKTVKKVQDKFRLDLSDEEAVHYMQSLIDESVHALFAAVVEQIHKFAQYWRK

SEQ ID NO: 26 - protein, 1068 aa, Homo sapiens
MPPRPSSGELWGIHLMPPRILVECLLPNGMIVTLECLREATLITIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPNVESSPELPKHIYNKLDKGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLMLMAKESLYSQLPMDCFTMPSYSRRISTATPYMNGETSTKSLWVINSALRIKILCATYVNVNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQEKKDETQKVQMKFLVEQMRRPDFMDALQGFLSPLNPAHQLGNLRLEECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALN

SEQ ID NO: 27 - protein, 1863 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 28 - protein, 3418 aa, Homo sapiens [sequence omitted]

SEQ ID NO: 29 - protein, 595 aa, Homo sapiens [sequence omitted]

SEQ ID NO: 30 - protein, 2839 aa, Homo sapiens
MAAHRPVEWVQAVVSRFDEQLPIKTGQQNTHTKVSTEHNKECLINISKYKFSLVISGLTTILKNVNNMRIFGEAAEKNLYLSQLIILDTLEKCLAGQPKDTMRLDETMLVKQLLPEICHFLHTCREGNQHAAELRNSASGVLFSLSCNNFNAVFSRISTRLQELTVCSEDNVDVHDIELLQYINVDCAKLKRLLKETAFKFKALKKVAQLAVINSLEKAFWNWVENYPDEFTKLYQIPQTDMAECAEKLFDLVDGFAESTKRKAAVWPLQIILLILCPEIIQDISKDVVDENNMNKKLFLDSLRKALAGHGGSRQLTESAAIACVKLCKASTYINWEDNSVIFLLVQSMVVDLKNLLFNPSKPFSRGSQPADVDLMIDCLVSCFRISPHNNQHFKICLAQNSPSTFHYVLVNSLHRIITNSALDWWPKIDAVYCHSVELRNMFGETLHKAVQGCGAHPAIRMAPSLTFKEKVTSLKFKEKPTDLETRSYKYLLLSMVKLIHADPKLLLCNPRKQGPETQGSTAELITGLVQLVPQSHMPEIAQEAMEALLVLHQLDSIDLWNPDAPVETFWEISSQMLFYICKKLTSHQMLSSTEILKWLREILICRNKFLLKNKQADRSSCHFLLFYGVGCDIPSSGNTSQMSMDHEELLRTPGASLRKGKGNSSMDSAAGCSGTPPICRQAQTKLEVALYMFLWNPDTEAVLVAMSCFRHLCEEADIRCGVDEVSVHNLLPNYNTFMEFASVSNMMSTGRAALQKRVMALLRRIEHPTAGNTEAWEDTHAKWEQATKLILNYPKAKMEDGQAAESLHKTIVKRRMSHVSGGGSIDLSDTDSLQEWINMTGFLCALGGVCLQQRSNSGLATYSPPMGPVSERKGSMISVMSSEGNADTPVSKFMDRLLSLMVCNHEKVGLQIRTNVKDLVGLELSPALYPMLFNKLKNTISKFFDSQGQVLLTDTNTQFVEQTIAIMKNLLDNHTEGSSEHLGQASIETMMLNLVRYVRVLGNMVHAIQIKTKLCQLVEVMMARRDDLSFCQEMKFRNKMVEYLTDWVMGTSNQAADDDVKCLTRDLDQASMEAVVSLLAGLPLQPEEGDGVELMEAKSQLFLKYFTLFMNLLNDCSEVEDESAQTGGRKRGMSRRLASLRHCTVLAMSNLLNANVDSGLMHSIGLGYHKDLQTRATFMEVLTKILQQGTEFDTLAETVLADRFERLVELVTMMGDQGELPIAMALANVVPCSQWDELARVLVTLFDSRHLLYQLLWNMFSKEVELADSMQTLFRGNSLASKIMTFCFKVYGATYLQKLLDPLLRIVITSSDWQHVSFEVDPTRLEPSESLEENQRNLLQMTEKFFHAIISSSSEFPPQLRSVCHCLYQATCHSLLNKATVKEKKENKKSVVSQRFPQNSIGAVGSAMFLRFINPAIVSPYEAGILDKKPPPRIERGLKLMSKILQSIANHVLFTKEEHMRPFNDFVKSNFDAARRFFLDIASDCPTSDAVNHSLSFISDGNVLALHRLLWNNQEKIGQYLSSNRDHKAVGRRPFDKMATLLAYLGPPEHKPVADTHWSSLNLTSSKFEEFMTRHQVHEKEEFKALKTLSIFYQAGTSKAGNPIFYYVARRFKTGQINGDLLIYHVLLTLKPYYAKPYEIVVDLTHTGPSNRFKTDFLSKWFVVFPGFAYDNVSAVYIYNCNSWVREYTKYHERLLTGLKGSKRLVFIDCPGKLAEHIEHEQQKLPAATLALEEDLKVFHNALKLAHKDTKVSIKVGSTAVQVTSAERTKVLGQSVFLNDIYYASEIEEICLVDENQFTLTIANQGTPLTFMHQECEAIVQSIIHIRTRWELSQPDSIPQHTKIRPKDVPGTLLNIALLNLGSSDPSLRSAAYNLLCALTCTFNLKIEGQLLETSGLCIPANNTLFIVSISKTLAANEPHLTLEFLEECISGFSKSSIELKHLCLEYMTPWLSNLVRFCKHNDDAKRQRVTAILDKLITMTINEKQMYPSIQAKIWGSLGQITDLLDVVLDSFIKTSATGGLGSIKAEVMADTAVALASGNVKLVSSKVIGRMCKIIDKTCLSPTPTLEQHLMWDDIAILARYMLMLSFNNSLDVAAHLPYLFHVVTFLVATGPLSLRASTHGLVINIIHSLCTCSQLHFSEETKQVLRLSLTEFSLPKFYLLFGISKVKSAAVIAFRSSYRDRSFSPGSYERETFALTSLETVTEALLEIMEACMRDIPTCKWLDQWTELAQRFAFQYNPSLQPRALVVFGCISKRVSHGQIKQIIRILSKALESCLKGPDTYNSQVLIEATVIALTKLQPLLNKDSPLHKALFWVAVAVLQLDEVNLYSAGTALLEQNLHTLDSLRIFNDKSPEEVFMAIRNPLEWHCKQMDHFVGLNFNSNFNFALVGHLLKGYRHPSPAIVARTVRILHTLLTLVNKHRNCDKFEVNTQSVAYLAALLTVSEEVRSRCSLKHRKSLLLTDISMENVPMDTYPIHHGDPSYRTLKETQPWSSPKGSEGYLAATYPTVGQTSPRARKSMSLDMGQPSQANTKKLLGTRKSFDHLISDTKAPKRQEMESGITTPPKMRRVAETDYEMETQRISSSQQHPHLRKVSVSESNVLLDEEVLTDPKIQALLLTVLATLVKYTTDEFDQRILYEYLAEASVVFPKVFPVVHNLLDSKINTLLSLCQDPNLLNPIHGIVQSVVYHEESPPQYQTSYLQSFGFNGLWRFAGPFSKQTQIPDYAELIVKFLDALIDTYLPGIDEETSEESLLTPTSPYPPALQSQLSITANLNLSNSMTSLATSQHSPGIDKENVELSPTTGHCNSGRTRHGSASQVQKQRSAGSFKRNSIKKIV

SEQ ID NO: 31 - protein, 593 aa, Homo sapiens
MAAHRPVEWVQAVVSRFDEQLPIKTGQQNTHTKVSTEHNKECLINISKYKFSLVISGLTTILKNVNNMRIFGEAAEKNLYLSQLIILDTLEKCLAGQPKDTMRLDETMLVKQLLPEICHFLHTCREGNQHAAELRNSASGVLFSLSCNNFNAVFSRISTRLQELTVCSEDNVDVHDIELLQYINVDCAKLKRLLKETAFKFKALKKVAQLAVINSLEKAFWNWVENYPDEFTKLYQIPQTDMAECAEKLFDLVDGFAESTKRKAAVWPLQIILLILCPEIIQDISKDVVDENNMNKKLFLDSLRKALAGHGGSRQLTESAAIACVKLCKASTYINWEDNSVIFLLVQSMVVDLKNLLFNPSKPFSRGSQPADVDLMIDCLVSCFRISPHNNQHFKICLAQNSPSTFHYVLVNSLHRIITNSALDWWPKIDAVYCHSVELRNMFGETLHKAVQGCGAHPAIRMAPSLTFKEKVTSLKFKEKPTDLETRSYKYLLLSMVKLIHADPKLLLCNPRKQGPETQGSTAELITGLVQLVPQSHMPEIAQEAMEALLVLHQLDSIDLWNPDAPVETFWEIRYMYFYFLNSTFKFYFVFLS

SEQ ID NO: 32 - protein, 928 aa, Homo sapiens
MPPKTPRKTAATAAAAAAEPPAPPPPPPPEEDPEQDSGPEDLPLVRLEFEETEEPDFTALCQKLKIPDHVRERAWLTWEKVSSVDGVLGGYIQKKKELWGICIFIAAVDLDEMSFTFTELQKNIEISVHKFFNLLKEIDTSTKVDNAMSRLLKKYDVLFALFSKLERTCELIYLTQPSSSISTEINSALVLKVSWITFLLAKGEVLQMEDDLVISFQLMLCVLDYFIKLSPPMLLKEPYKTAVIPINGSPRTPRRGQNRSARIAKQLENDTRIIEVLCKEHECNIDEVKNVYFKNFIPFMNSLGLVTSNGLPEVENLSKRYEEIYLKNKDLDARLFLDHDKTLQTDSIDSFETQRTPRKSNLDEEVNVIPPHTPVRTVMNTIQQLMMILNSASDQPSENLISYFNNCTVNPKESILKRVKDIGYIFKEKFAKAVGQGCVEIGSQRYKLGVRLYYRVMESMLKSEEERLSIQNFSKLLNDNIFHMSLLACALEVVMATYSRSTSQNLDSGTDLSFPWILNVLNLKAFDFYKVIESFIKAEGNLTREMIKHLERCEHRIMESLAWLSDSPLFDLIKQSKDREGPTDHLESACPLNLPLQNNHTAADMYLSPVRSPKKKGSTTRVNSTANAETQATSAFQTQKPLKSTSLSLFYKKVYRLAYLRLNTLCERLLSEHPELEHIIWTLFQHTLQNEYELMRDRHLDQIMMCSMYGICKVKNIDLKFKIIVTAYKDLPHAVQETFKRVLIKEEEYDSIIVFYNSVFMQRLKTNILQYASTRPPTLSPIPHIPRSPYKFPSSPLRIPGGNIYISPLKSPYKISEGLPTPTKMTPRSRILVSIGESFGTSEKFQKINQMVCNSDRVLKRSAEGSNPPKPLKKLRFDIEGSDEADGSKHLPGESKFQQKLAEMTSTRTRMQKQKMNDSMDTSNKEEK

SEQ ID NO: 33 - protein, 188 aa, Homo sapiens [sequence omitted]

SEQ ID NO: 34 - protein, 1255 aa, Homo sapiens [sequence omitted]

SEQ ID NO: 35 - protein, 766 aa, Homo sapiens [sequence omitted]

SEQ ID NO: 36 - protein, 2285 aa, Homo sapiens
MAAQVAPAAASSLGNPPPPPPSELKKAEQQQREEAGGEAAAAAAAERGEMKAAAGQESEGPAVGPPQPLGKELQDGAESNGGGGGGGAGSGGGPGAEPDLKNSNGNAGPRPALNNNLTEPPGGGGGGSSDGVGAPPHSAAAALPPPAYGFGQPYGRSPSAVAAAAAAVFHQQHGGQQSPGLAALQSGGGGGLEPYAGPQQNSHDHGFPNHQYNSYYPNRSAYPPPAPAYALSSPRGGTPGSGAAAAAGSKPPPSSSASASSSSSSFAQQRFGAMGGGGPSAAGGGTPQPTATPTLNQLLTSPSSARGYQGYPGGDYSGGPQDGGAGKGPADMASQCWGAAAAAAAAAAASGGAQQRSHHAPMSPGSSGGGGQPLARTPQPSSPMDQMGKMRPQPYGGTNPYSQQQGPPSGPQQGHGYPGQPYGSQTPQRYPMTMQGRAQSAMGGLSYTQQIPPYGQQGPSGYGQQGQTPYYNQQSPHPQQQQPPYSQQPPSQTPHAQPSYQQQPQSQPPQLQSSQPPYSQQPSQPPHQQSPAPYPSQQSTTQQHPQSQPPYSQPQAQSPYQQQQPQQPAPSTLSQQAAYPQPQSQQSQQTAYSQQRFPPPQELSQDSFGSQASSAPSMTSSKGGQEDMNLSLQSRPSSLPDLSGSIDDLPMGTEGALSPGVSTSGISSSQGEQSNPAQSPFSPHTSPHLPGIRGPSPSPVGSPASVAQSRSGPLSPAAVPGNQMPPRPPSGQSDSIMHPSMNQSSIAQDRGYMQRNPQMPQYSSPQPGSALSPRQPSGGQIHTGMGSYQQNSMGSYGPQGGQYGPQGGYPRQPNYNALPNANYPSAGMAGGINPMGAGGQMHGQPGIPPYGTLPPGRMSHASMGNRPYGPNMANMPPQVGSGMCPPPGGMNRKTQETAVAMHVAANSIQNRPPGYPNMNQGGMMGTGPPYGQGINSMAGMINPQGPPYSMGGTMANNSAGMAASPEMMGLGDVKLTPATKMNNKADGTPKTESKSKKSSSSTTTNEKITKLYELGGEPERKMWVDRYLAFTEEKAMGMTNLPAVGRKPLDLYRLYVSVKEIGGLTQVNKNKKWRELATNLNVGTSSSAASSLKKQYIQCLYAFECKIERGEDPPPDIFAAADSKKSQPKIQPPSPAGSGSMQGPQTPQSTSSSMAEGGDLKPPTPASTPHSQIPPLPGMSRSNSVGIQDAFNDGSDSTFQKRNSMTPNPGYQPSMNTSDMMGRMSYEPNKDPYGSMRKAPGSDPFMSSGQGPNGGMGDPYSRAAGPGLGNVAMGPRQHYPYGGPYDRVRTEPGIGPEGNMSTGAPQPNLMPSNPDSGMYSPSRYPPQQQQQQQQRHDSYGNQFSTQGTPSGSPFPSQQTTMYQQQQQNYKRPMDGTYGPPAKRHEGEMYSVPYSTGQGQPQQQQLPPAQPQPASQQQAAQPSPQQDVYNQYGNAYPATATAATERRPAGGPQNQFPFQFGRDRVSAPPGTNAQQNMPPQMMGGPIQASAEVAQQGTMWQGRNDMTYNYANRQSTGSAPQGPAYHGVNRTDEMLHTDQRANHEGSWPSHGTRQPPYGPSAPVPPMTRPPPSNYQPPPSMQNHIPQVSSPAPLPRPMENRTSPSKSPFLHSGMKMQKAGPPVPASHIAPAPVQPPMIRRDITFPPGSVEATQPVLKQRRRLTMKDIGTPEAWRVMMSLKSGLLAESTWALDTINILLYDDNSIMTFNLSQLPGLLELLVEYFRRCLIEIFGILKEYEVGDPGQRTLLDPGRFSKVSSPAPMEGGEEEEELLGPKLEEEEEEEVVENDEEIAFSGKDKPASENSEEKLISKFDKLPVKIVQKNDPFVVDCSDKLGRVQEFDSGLLHWRIGGGDTTEHIQTHFESKTELLPSRPHAPCPPAPRKHVTTAEGTPGTTDQEGPPPDGPPEKRITATMDDMLSTRSSTLTEDGAKSSEAIKESSKFPFGISPAQSHRNIKILEDEPHSKDETPLCTLLDWQDSLAKRCVCVSNTIRSLSFVPGNDFEMSKHPGLLLILGKLILLHHKHPERKQAPLTYEKEEEQDQGVSCNKVEWWWDCLEMLRENTLVTLANISGQLDLSPYPESICLPVLDGLLHWAVCPSAEAQDPFSTLGPNAVLSPQRLVLETLSKLSIQDNNVDLILATPPFSRLEKLYSTMVRFLSDRKNPVCREMAVVLLANLAQGDSLAARAIAVQKGSIGNLLGFLEDSLAATQFQQSQASLLHMQNPPFEPTSVDMMRRAARALLALAKVDENHSEFTLYESRLLDISVSPLMNSLVSQVICDVLFLIGQS

SEQ ID NO: 37 - protein, 403 aa, Homo sapiens [sequence omitted]

SEQ ID NO: 38 - protein, 821 aa, Homo sapiens
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEEYLDLSQPLEQYSPSYPDTRSSCSSGDDSVFSPDPMPYEPCLPQYPHINGSVKT

SEQ ID NO: 39 - protein, 2440 aa, Homo sapiens
MSSSGYPPNQGAFSTEQSRYPPHSVQYTFPNTRHQQEFAVPDYRSSHLEVSQASQLLQQQQQQQLRRRPSLLSEFHPGSDRPQERRTSYEPFHPGPSPVDHDSLESKRPRLEQVSDSHFQRVSAAVLPLVHPLPEGLRASADAKKDPAFGGKHEAPSSPISGQPCGDDQNASPSKLSKEELIQSMDRVDREIAKVEQQILKLKKKQQQLEEEAAKPPEPEKPVSPPPVEQKHRSIVQIIYDENRKKAEEAHKIFEGLGPKVELPLYNQPSDTKVYHENIKTNQVMRKKLILFFKRRNHARKQREQKICQRYDQLMEAWEKKVDRIENNPRRKAKESKTREYYEKQFPEIRKQREQQERFQRVGQRGAGLSATIARSEHEISEIIDGLSEQENNEKQMRQLSVIPPMMFDAEQRRVKFINMNGLMEDPMKVYKDRQFMNVWTDHEKEIFKDKFIQHPKNFGLIASYLERKSVPDCVLYYYLTKKNENYKALVRRNYGKRRGRNQQIARPSQEEKVEEKEEDKAEKTEKKEEEKKDEEEKDEKEDSKENTKEKDKIDGTAEETEEREQATPRGRKTANSQGRRKGRITRSMTNEAAAASAAAAAATEEPPPPLPPPPEPISTEPVETSRWTEEEMEVAKKGLVEHGRNWAAIAKMVGTKSEAQCKNFYFNYKRRHNLDNLLQQHKQKTSRKPREERDVSQCESVASTVSAQEDEDIEASNEEENPEDSEVEAVKPSEDSPENATSRGNTEPAVELEPTTETAPSTSPSLAVPSTKPAEDESVETQVNDSISAETAEQMDVDQQEHSAEEGSVCDPPPATKADSVDVEVRVPENHASKVEGDNTKERDLDRASEKVEPRDEDLVVAQQINAQRPEPQSDNDSSATCSADEDVDGEPERQRMFPMDSKPSLLNPTGSILVSSPLKPNPLDLPQLQHRAAVIPPMVSCTPCNIPIGTPVSGYALYQRHIKAMHESALLEEQRQRQEQIDLECRSSTSPCGTSKSPNREWEVLQPAPHQVITNLPEGVRLPTTRPTRPPPPLIPSSKTTVASEKPSFIMGGSISQGTPGTYLTSHNQASYTQETPKPSVGSISLGLPRQQESAKSATLPYIKQEEFSPRSQNSQPEGLLVRAQHEGVVRGTAGAIQEGSITRGTPTSKISVESIPSLRGSITQGTPALPQTGIPTEALVKGSISRMPIEDSSPEKGREEAASKGHVIYEGKSGHILSYDNIKNAREGTRSPRTAHEISLKRSYESVEGNIKQGMSMRESPVSAPLEGLICRALPRGSPHSDLKERTVLSGSIMQGTPRATTESFEDGLKYPKQIKRESPPIRAFEGAITKGKPYDGITTIKEMGRSIHEIPRQDILTQESRKTPEVVQSTRPIIEGSISQGTPIKFDNNSGQSAIKHNVKSLITGPSKLSRGMPPLEIVPENIKVVERGKYEDVKAGETVRSRHTSVVSSGPSVLRSTLHEAPKAQLSPGIYDDTSARRTPVSYQNTMSRGSPMMNRTSDVTISSNKSTNHERKSTLTPTQRESIPAKSPVPGVDPVVSHSPFDPHHRGSTAGEVYRSHLPTHLDPAMPFHRALDPAAAAYLFQRQLSPTPGYPSQYQLYAMENTRQTILNDYITSQQMQVNLRPDVARGLSPREQPLGLPYPATRGIIDLTNMPPTILVPHPGGTSTPPMDRITYIPGTQITFPPRPYNSASMSPGHPTHLAAAASAEREREREREKERERERIAAASSDLYLRPGSEQPGRPGSHGYVRSPSPSVRTQETMLQQRPSVFQGTNGTSVITPLDPTAQLRIMPLPAGGPSISQGLPASRYNTAADALAALVDAAASAPQMDVSKTKESKHEAARLEENLRSRSAAVSEQQQLEQKTLEVEKRSVQCLYTSSAFPSGKPQPHSSVVYSEAGKDKGPPPKSRYEEELRTRGKTTITAANFIDVIITRQIASDKDARERGSQSSDSSSSLSSHRYETPSDAIEVISPASSPAPPQEKLQTYQPEVVKANQAENDPTRQYEGPLHHYRPQQESPSPQQQLPPSSQAEGMGQVPRTHRLITLADHICQIITQDFARNQVSSQTPQQPPTSTFQNSPSALVSTPVRTKTSNRYSPESQAQSVHHQRPGSRVSPENLVDKSRGSRPGKSPERSHVSSEPYEPISPPQVPVVHEKQDSLLLLSQRGAEPAEQRNDARSPGSISYLPSFFTKLENTSPMVKSKKQEIFRKLNSSGGGDSDMAAAQPGTEIFNLPAVTTSGSVSSRGHSFADPASNLGLEDIIRKALMGSFDDKVEDHGVVMSQPMGVVPGTANTSVVTSGETRREEGDPSPHSGGVCKPKLISKSNSRKSKSPIPGQGYLGTERPSSVSSVHSEGDYHRQTPGWAWEDRPSSTGSTQFPYNPLTMRMLSSTPPTPIACAPSAVNQAAPHQQNRIWEREPAPLLSAQYETLSDSDD

SEQ ID NO: 40 - protein, 552 aa, Homo sapiens
MDNMSITNTPTSNDACLSIVHSLMCHRQGGESETFAKRAIESLVKKLKEKKDELDSLITAITTNGAHPSKCVTIQRTLDGRLQVAGRKGFPHVIYARLWRWPDLHKNELKHVKYCQYAFDLKCDSVCVNPYHYERVVSPGIDLSGLTLQSNAPSSMMVKDEYVHDFEGQPSLSTEGHSIQTIQHPPSNRASTETYSTPALLAPSESNATSTANFPNIPVASTSQPASILGGSHSEGLLQIASGPQPGQQQNGFTGQPATYHHNSTTTWTGSRTAPYTPNLPHHQNGHLQHHPPMPPHPGHYWPVHNELAFQPPISNHPAPEYWCSIAYFEMDVQVGETFKVPSSCPIVTVDGYVDPSGGDRFCLGQLSNVHRTEAIERARLHIGKGVQLECKGEGDVWVRCLSDHAVFVQSYYLDREAGRAPGDAVHKIYPSAYIKVFDLRQCHRQMQQQAATAQAAAAAQAAAVAGNIPGPGSVGGIAPAISLSAAAGIGVDDLRRLCILRMSFVKGWGPDYPRQSIKETPCWIEIHLHRALQLLDEVLHTMPIADPQPLD

SEQ ID NO: 41 - protein, 677 aa, Homo sapiens
MMQESGTETKSNGSAIQNGSGGSNHLLECGGLREGRSNGETPAVDIGAADLAHAQQQQQQALQVARQLLLQQQQQQQVSGLKSPKRNDKQPALQVPVSVAMMTPQVITPQQMQQILQQQVLSPQQLQVLLQQQQALMLQQQQLQEFYKKQQEQLQLQLLQQQHAGKQPKEQQQVATQQLAFQQQLLQMQQLQQQHLLSLQRQGLLTIQPGQPALPLQPLAQGMIPTELQQLWKEVTSAHTAEETTGNNHSSLDLTTTCVSSSAPSKTSLIMNPHASTNGQLSVHTPKRESLSHEEHPHSHPLYGHGVCKWPGCEAVCEDFQSFLKHLNSEHALDDRSTAQCRVQMQVVQQLELQLAKDKERLQAMMTHLHVKSTEPKAAPQPLNLVSSVTLSKSASEASPQSLPHTPTTPTAPLTPVTQGPSVITTTSMHTVGPIRRRYSDKYNVPISSADIAQNQEFYKNAEVRPPFTYASLIRQAILESPEKQLTLNEIYNWFTRMFAYFRRNAATWKNAVRHNLSLHKCFVRVENVKGAVWTVDEVEFQKRRPQKISGNPSLIKNMQSSHAYCTPLNAALQASMAENSIPLYTTASMGNPTLGNLASAIREELNGAMEHTNSNESDSSPGRSPMQAVHPVHVKEEPLDPEEAEGPLSLVTTANHSPDFDHDRDYEDEPVNEDME

SEQ ID NO: 42 - protein, 2843 aa, Homo sapiens
MAAASYDQLLKQVEALKMENSNLRQELEDNSNHLTKLETEASNMKEVLKQLQGSIEDEAMASSGQIDLLERLKELNLDSSNFPGVKLRSKMSLRSYGSREGSVSSRSGECSPVPMGSFPRRGFVNGSRESTGYLEELEKERSLLLADLDKEEKEKDWYYAQLQNLTKRIDSLPLTENFSLQTDMTRRQLEYEARQIRVAMEEQLGTCQDMEKRAQRRIARIQQIEKDILRIRQLLQSQATEAERSSQNKHETGSHDAERQNEGQGVGEINMATSGNGQGSTTRMDHETASVLSSSSTHSAPRRLTSHLGTKVEMVYSLLSMLGTHDKDDMSRTLLAMSSSQDSCISMRQSGCLPLLIQLLHGNDKDSVLLGNSRGSKEARARASAALHNIIHSQPDDKRGRREIRVLHLLEQIRAYCETCWEWQEAHEPGMDQDKNPMPAPVEHQICPAVCVLMKLSFDEEHRHAMNELGGLQAIAELLQVDCEMYGLTNDHYSITLRRYAGMALTNLTFGDVANKATLCSMKGCMRALVAQLKSESEDLQQVIASVLRNLSWRADVNSKKTLREVGSVKALMECALEVKKESTLKSVLSALWNLSAHCTENKADICAVDGALAFLVGTLTYRSQTNTLAIIESGGGILRNVSSLIATNEDHRQILRENNCLQTLLQHLKSHSLTIVSNACGTLWNLSARNPKDQEALWDMGAVSMLKNLIHSKHKMIAMGSAAALRNLMANRPAKYKDANIMSPGSSLPSLHVRKQKALEAELDAQHLSETFDNIDNLSPKASHRSKQRHKQSLYGDYVFDTNRHDDNRSDNFNTGNMTVLSPYLNTTVLPSSSSSRGSLDSSRSEKDRSLERERGIGLGNYHPATENPGTSSKRGLQISTTAAQIAKVMEEVSAIHTSQEDRSSGSTTELHCVTDERNALRRSSAAHTHSNTYNFTKSENSNRTCSMPYAKLEYKRSSNDSLNSVSSSDGYGKRGQMKPSIESYSEDDESKFCSYGQYPADLAHKIHSANHMDDNDGELDTPINYSLKYSDEQLNSGRQSPSQNERWARPKHIIEDEIKQSEQRQSRNQSTTYPVYTESTDDKHLKFQPHFGQQECVSPYRSRGANGSETNRVGSNHGINQNVSQSLCQEDDYEDDKPTNYSERYSEEEQHEEEERPTNYSIKYNEEKRHVDQPIDYSLKYATDIPSSQKQSFSFSKSSSGQSSKTEHMSSSSENTSTPSSNAKRQNQLHPSSAQSRSGQPQKAATCKVSSINQETIQTYCVEDTPICFSRCSSLSSLSSAEDEIGCNQTTQEADSANTLQIAEIKEKIGTRSAEDPVSEVPAVSQHPRTKSSRLQGSSLSSESARHKAVEFSSGAKSPSKSGAQTPKSPPEHYVQETPLMFSRCTSVSSLDSFESRSIASSVQSEPCSGMVSGIISPSDLPDSPGQTMPPSRSKTPPPPPQTAQTKREVPKNKAPTAEKRESGPKQAAVNAAVQRVQVLPDADTLLHFATESTPDGFSCSSSLSALSLDEPFIQKDVELRIMPPVQENDNGNETESEQPKESNENQEKEAEKTIDSEKDLLDDSDDDDIEILEECIISAMPTKSSRKAKKPAQTASKLPPPVARKPSQLPVYKLLPSQNRLQPQKHVSFTPGDDMPRVYCVEGTPINFSTATSLSDLTIESPPNELAAGEGVRGGAQSGEFEKRDTIPTEGRSTDEAQGGKTSSVTIPELDDNKAEEGDILAECINSAMPKGKSHKPFRVKKIMDQVQQASASSSAPNKNQLDGKKKKPTSPVKPIPQNTEYRTRVRKNADSKNNLNAERVFSDNKDSKKQNLKNNSKVFNDKLPNNEDRVRGSFAFDSPHHYTPIEGTPYCFSRNDSLSSLDFDDDDVDLSREKAELRKAKENKESEAKVTSHTELTSNQQSANKTQAIAKQPINRGQPKPILQKQSTFPQSSKDIPDRGAATDEKLQNFAIENTPVCFSHNSSLSSLSDIDQENNNKENEPIKETEPPDSQGEPSKPQASGYAPKSFHVEDTPVCFSRNSSLSSLSIDSEDDLLQECISSAMPKKKKPSRLKGDNEKHSPRNMGGILGEDLTLDLKDIQRPDSEHGLSPDSENFDWKAIQEGANSIVSSLHQAAAAACLSRQASSDSDSILSLKSGISLGSPFHLTPDQEEKPFTSNKGPRILKPGEKSTLETKKIESESKGIKGGKKVYKSLITGKVRSNSEISGQMKQPLQANMPSISRGRTMIHIPGVRNSSSSTSPVSKKGPPLKTPASKSPSEGQTATTSPRGAKPSVKSELSPVARQTSQIGGSSKAPSRSGSRDSTPSRPAQQPLSRPIQSPGRNSISPGRNGISPPNKLSQLPRTSSPSTASTKSSGSGKMSYTSPGRQMSQQNLTKQTGLSKNASSIPRSESASKGLNQMNNGNGANKKVELSRMSSTKSSGSESDRSERPVLVRQSTFIKEAPSPTLRRKLEESASFESLSPSSRPASPTRSQAQTPVLSPSLPDMSLSTHSSVQAGGWRKLPPNLSPTIEYNDGRPAKRHDIARSHSESPSRLPINRSGTWKREHSKHSSSLPRVSTWRRTGSSSSILSASSESSEKAKSEDEKHVNSISGTKQSKENQVSAKGTWRKIKENEFSPTNSTSQTVSSGATNGAESKTLIYQMAPAVSKTEDVWVRIEDCPINNPRSGRSPTGNTPPVIDSVSEKANPNIKDSKDNQAKQNVGNGSVPMRTVGLENRLNSFIQVDAPDQKGTEIKPGQNNPVPVSETNESSIVERTPFSSSSSSKHSSPSGTVAARVTPFNYNPSPRKSSADSTSARPSQIPTPVNNNTKKRDSKTDSTESSGTQSPKRHSGSYLVTSV

SEQ ID NO: 43 - protein, 509 aa, Homo sapiens
MNLLDPFMKMTDEQEKGLSGAPSPTMSEDSAGSPCPSGSGSDTENTRPQENTFPKGEPDLKKESEEDKFPVCIREAVSQVLKGYDWTLVPMPVRVNGSSKNKPHVKRPMNAFMVWAQAARRKLADQYPHLHNAELSKTLGKLWRLLNESEKRPFVEEAERLRVQHKKDHPDYKYQPRRRKSVKNGQAEAEEATEQTHISPNAIFKALQADSPHSSSGMSEVHSPGEHSGQSQGPPTPPTTPKTDVQPGKADLKREGRPLPEGGRQPPIDFRDVDIGELSSDVISNIETFDVNEFDQYLPPNGHPGVPATHGQVTYTGSYGISSTAATPASAGHVWMSKQQAPPPPPQQPPQAPPAPQAPPQPQAAPPQQPAAPPQQPQAHTLTTLSSEPGQSQRTHIKTEQLSPSHYSEQQQHSPQQIAYSPFNLPHYSPSYPPITRSQYDYTDHQNSSSYYSHAAGQGTGLYSTFTYMNPAQRPMYTPIADTSGVPSIPQTHSPQHWEQPVYTQLTRP

SEQ ID NO: 44 - protein, 496 aa, Homo sapiens
MDFSRNLYDIGEQLDSEDLASLKFLSLDYIPQRKQEPIKDALMLFQRLQEKRMLEESNLSFLKELLFRINRLDLLITYLNTRKEEMERELQTPGRAQISAYRFHFCRMSWAEANSQCQTQSVPFWRRVDHLLIRVMLYQISEEVSRSELRSFKFLLQEEISKCKLDDDMNLLDIFIEMEKRVILGEGKLDILKRVCAQINKSLLKIINDYEEFSKGEELCGVMTISDSPREQDSESQTLDKVYQMKSKPRGYCLIINNHNFAKAREKVPKLHSIRDRNGTHLDAGALTTTFEELHFEIKPHDDCTVEQIYEILKIYQLMDHSNMDCFICCILSHGDKGIIYGTDGQEAPIYELTSQFTGLKCPSLAGKPKVFFIQACQGDNYQKGIPVETDSEEQPYLEMDLSSPQTRYIPDEADFLLGMATVNNCVSYRNPAEGTWYIQSLCQSLRERCPRGDDILTILTEVNYEVSNKDDKKNMGKQMPQPTFTLRKKLVFPSD

SEQ ID NO: 45 - protein, 593 aa, Homo sapiens [sequence omitted]

SEQ ID NO: 46 - protein, 1132 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 47 - protein, 1620 aa, Homo sapiens
MGAIGLLWLLPLLLSTAAVGSGMGTGQRAGSPAAGPPLQPREPLSYSRLQRKSLAVDFVVPSLFRVYARDLLLPPSSSELKAGRPEARGSLALDCAPLLRLLGPAPGVSWTAGSPAPAEARTLSRVLKGGSVRKLRRAKQLVLELGEEAILEGCVGPPGEAAVGLLQFNLSELFSWWIRQGEGRLRIRLMPEKKASEVGREGRLSAAIRASQPRLLFQIFGTGHSSLESPTNMPSPSPDYFTWNLTWIMKDSFPFLSHRSRYGLECSFDFPCELEYSPPLHDLRNQSWSWRRIPSEEASQMDLLDGPGAERSKEMPRGSFLLLNTSADSKHTILSPWMRSSSEHCTLAVSVHRHLQPSGRYIAQLLPHNEAAREILLMPTPGKHGWTVLQGRIGRPDNPFRVALEYISSGNRSLSAVDFFALKNCSEGTSPGSKMALQSSFTCWNGTVLQLGQACDFHQDCAQGEDESQMCRKLPVGFYCNFEDGFCGWTQGTLSPHTPQWQVRTLKDARFQDHQDHALLLSTTDVPASESATVTSATFPAPIKSSPCELRMSWLIRGVLRGNVSLVLVENKTGKEQGRMVWHVAAYEGLSLWQWMVLPLLDVSDRFWLQMVAWWGQGSRAIVAFDNISISLDCYLTISGEDKILQNTAPKSRNLFERNPNKELKPGENSPRQTPIFDPTVHWLFTTCGASGPHGPTQAQCNNAYQNSNLSVEVGSEGPLKGIQIWKVPATDTYSISGYGAAGGKGGKNTMMRSHGVSVLGIFNLEKDDMLYILVGQQGEDACPSTNQLIQKVCIGENNVIEEEIRVNRSVHEWAGGGGGGGGATYVFKMKDGVPVPLIIAAGGGGRAYGAKTDTFHPERLENNSSVLGLNGNSGAAGGGGGWNDNTSLLWAGKSLQEGATGGHSCPQAMKKWGWETRGGFGGGGGGCSSGGGGGGYIGGNAASNNDPEMDGEDGVSFISPLGILYTPALKVMEGHGEVNIKHYLNCSHCEVDECHMDPESHKVICFCDHGTVLAEDGVSCIVSPTPEPHLPLSLILSVVTSALVAALVLAFSGIMIVYRRKHQELQAMQMELQSPEYKLSKLRTSTIMTDYNPNYCFAGKTSSISDLKEVPRKNITLIRGLGHGAFGEVYEGQVSGMPNDPSPLQVAVKTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALPIEYGPLVEEEEKVPVRPKDPEGVPPLLVSQQAKREEERSPAAPPPLPTTSSGKAAKKPTAAEISVRVPRGPAVEGGHVNMAFSQSNPPSELHKVHGSRNKPTSLWNPTYGSWFTEKPTKKNNPIAKKEPHDRGNLGLEGSCTVPPNVATGRLPGASLLLEPSSLTANMKEVPLFRLRHFPCGNVNYGYQQQGLPLEAATAPGAGHYEDTILKSKNSMNQPGP

SEQ ID NO: 48 - protein, 5537 aa, Homo sapiens
MDSQKLAGEDKDSEPAADGPAASEDPSATESDLPNPHVGEVSVLSSGSPRLQETPQDCSGGPVRRCALCNCGEPSLHGQRELRRFELPFDWPRCPVVSPGGSPGPNEAVLPSEDLSQIGFPEGLTPAHLGEPGGSCWAHHWCAAWSAGVWGQEGPELCGVDKAIFSGISQRCSHCTRLGASIPCRSPGCPRLYHFPCATASGSFLSMKTLQLLCPEHSEGAAYLEEARCAVCEGPGELCDLFFCTSCGHHYHGACLDTALTARKRAGWQCPECKVCQACRKPGNDSKMLVCETCDKGYHTFCLKPPMEELPAHSWKCKACRVCRACGAGSAELNPNSEWFENYSLCHRCHKAQGGQTIRSVAEQHTPVCSRFSPPEPGDTPTDEPDALYVACQGQPKGGHVTSMQPKEPGPLQCEAKPLGKAGVQLEPQLEAPLNEEMPLLPPPEESPLSPPPEESPTSPPPEASRLSPPPEELPASPLPEALHLSRPLEESPLSPPPEESPLSPPPESSPFSPLEESPLSPPEESPPSPALETPLSPPPEASPLSPPFEESPLSPPPEELPTSPPPEASRLSPPPEESPMSPPPEESPMSPPPEASRLFPPFEESPLSPPPEESPLSPPPEASRLSPPPEDSPMSPPPEESPMSPPPEVSRLSPLPVVSRLSPPPEESPLSPPPEESPTSPPPEASRLSPPPEDSPTSPPPEDSPASPPPEDSLMSLPLEESPLLPLPEEPQLCPRSEGPHLSPRPEEPHLSPRPEEPHLSPQAEEPHLSPQPEEPCLCAVPEEPHLSPQAEGPHLSPQPEELHLSPQTEEPHLSPVPEEPCLSPQPEESHLSPQSEEPCLSPRPEESHLSPELEKPPLSPRPEKPPEEPGQCPAPEELPLFPPPGEPSLSPLLGEPALSEPGEPPLSPLPEELPLSPSGEPSLSPQLMPPDPLPPPLSPIITAAAPPALSPLGELEYPFGAKGDSDPESPLAAPILETPISPPPEANCTDPEPVPPMILPPSPGSPVGPASPILMEPLPPQCSPLLQHSLVPQNSPPSQCSPPALPLSVPSPLSPIGKVVGVSDEAELHEMETEKVSEPECPALEPSATSPLPSPMGDLSCPAPSPAPALDDFSGLGEDTAPLDGIDAPGSQPEPGQTPGSLASELKGSPVLLDPEELAPVTPMEVYPECKQTAGQGSPCEEQEEPRAPVAPTPPTLIKSDIVNEISNLSQGDASASFPGSEPLLGSPDPEGGGSLSMELGVSTDVSPARDEGSLRLCTDSLPETDDSLLCDAGTAISGGKAEGEKGRRRSSPARSRIKQGRSSSFPGRRRPRGGAHGGRGRGRARLKSTASSIETLVVADIDSSPSKEEEEEDDDTMQNTVVLFSNTDKFVLMQDMCVVCGSFGRGAEGHLLACSQCSQCYHPYCVNSKITKVMLLKGWRCVECIVCEVCGQASDPSRLLLCDDCDISYHTYCLDPPLLTVPKGGWKCKWCVSCMQCGAASPGFHCEWQNSYTHCGPCASLVTCPICHAPYVEEDLLIQCRHCERWMHAGCESLFTEDDVEQAADEGFDCVSCQPYVVKPVAPVAPPELVPMKVKEPEPQYFRFEGVWLTETGMALLRNLTMSPLHKRRQRRGRLGLPGEAGLEGSEPSDALGPDDKKDGDLDTDELLKGEGGVEHMECEIKLEGPVSPDVEPGKEETEESKKRKRKPYRPGIGGFMVRQRKSHTRTKKGPAAQAEVLSGDGQPDEVIPADLPAEGAVEQSLAEGDEKKKQQRRGRKKSKLEDMFPAYLQEAFFGKELLDLSRKALFAVGVGRPSFGLGTPKAKGDGGSERKELPTSQKGDDGPDIADEESRGLEGKADTPGPEDGGVKASPVPSDPEKPGTPGEGMLSSDLDRISTEELPKMESKDLQQLFKDVLGSEREQHLGCGTPGLEGSRTPLQRPFLQGGLPLGNLPSSSPMDSYPGLCQSPFLDSRERGGFFSPEPGEPDSPWTGSGGTTPSTPTTPTTEGEGDGLSYNQRSLQRWEKDEELGQLSTISPVLYANINFPNLKQDYPDWSSRCKQIMKLWRKVPAADKAPYLQKAKDNRAAHRINKVQKQAESQINKQTKVGDIARKTDRPALHLRIPPQPGALGSPPPAAAPTIFIGSPTTPAGLSTSADGFLKPPAGSVPGPDSPGELFLKLPPQVPAQVPSQDPFGLAPAYPLEPRFPTAPPTYPPYPSPTGAPAQPPMLGASSRPGAGQPGEFHTTPPGTPRHQPSTPDPFLKPRCPSLDNLAVPESPGVGGGKASEPLLSPPPFGESRKALEVKKEELGASSPSYGPPNLGFVDSPSSGTHLGGLELKTPDVFKAPLTPRASQVEPQSPGLGLRPQEPPPAQALAPSPPSHPDIFRPGSYTDPYAQPPLTPRPQPPPPESCCALPPRSLPSDPFSRVPASPQSQSSSQSPLTPRPLSAEAFCPSPVTPRFQSPDPYSRPPSRPQSRDPFAPLHKPPRPQPPEVAFKAGSLAHTSLGAGGFPAALPAGPAGELHAKVPSGQPPNFVRSPGTGAFVGTPSPMRFTFPQAVGEPSLKPPVPQPGLPPPHGINSHFGPGPTLGKPQSTNYTVATGNFHPSGSPLGPSSGSTGESYGLSPLRPPSVLPPPAPDGSLPYLSHGASQRSGITSPVEKREDPGTGMGSSLATAELPGTQDPGMSGLSQTELEKQRQRQRLRELLIRQQIQRNTLRQEKETAAAAAGAVGPPGSWGAEPSSPAFEQLSRGQTPFAGTQDKSSLVGLPPSKLSGPILGPGSFPSDDRLSRPPPPATPSSMDVNSRQLVGGSQAFYQRAPYPGSLPLQQQQQQLWQQQQATAATSMRFAMSARFPSTPGPELGRQALGSPLAGISTRLPGPGEPVPGPAGPAQFIELRHNVQKGLGPGGTPFPGQGPPQRPRFYPVSEDPHRLAPEGLRGLAVSGLPPQKPSAPPAPELNNSLHPTPHTKGPTLPTGLELVNRPPSSTELGRPNPLALEAGKLPCEDPELDDDFDAHKALEDDEELAHLGLGVDVAKGDDELGTLENLETNDPHLDDLLNGDEFDLLAYTDPELDTGDKKDIFNEHLRLVESANEKAEREALLRGVEPGPLGPEERPPPAADASEPRLASVLPEVKPKVEEGGRHPSPCQFTIATPKVEPAPAANSLGLGLKPGQSMMGSRDTRMGTGPFSSSGHTAEKASFGATGGPPAHLLTPSPLSGPGGSSLLEKFELESGALTLPGGPAASGDELDKMESSLVASELPLLIEDLLEHEKKELQKKQQLSAQLQPAQQQQQQQQQHSLLSAPGPAQAMSLPHEGSSPSLAGSQQQLSLGLAGARQPGLPQPLMPTQPPAHALQQRLAPSMAMVSNQGHMLSGQHGGQAGLVPQQSSQPVLSQKPMGTMPPSMCMKPQQLAMQQQLANSFFPDTDLDKFAAEDIIDPIAKAKMVALKGIKKVMAQGSIGVAPGMNRQQVSLLAQRLSGGPSSDLQNHVAAGSGQERSAGDPSQPRPNPPTFAQGVINEADQRQYEEWLFHTQQLLQMQLKVLEEQIGVHRKSRKALCAKQRTAKKAGREFPEADAEKLKLVTEQQSKIQKQLDQVRKQQKEHTNLMAEYRNKQQQQQQQQQQQQQQHSAVLALSPSQSPRLLTKLPGQLLPGHGLQPPQGPPGGQAGGLRLTPGGMALPGQPGGPFLNTALAQQQQQQHSGGAGSLAGPSGGFFPGNLALRSLGPDSRLLQERQLQLQQQRMQLAQKLQQQQQQQQQQQHLLGQVAIQQQQQQGPGVQTNQALGPKPQGLMPPSSHQGLLVQQLSPQPPQGPQGMLGPAQVAVLQQQHPGALGPQGPHRQVLMTQSRVLSSPQLAQQGQGLMGHRLVTAQQQQQQQQHQQQGSMAGLSHLQQSLMSHSGQPKLSAQPMGSLQQLQQQQQLQQQQQLQQQQQQQLQQQQQLQQQQLQQQQQQQQLQQQQQQQLQQQQQQLQQQQQQQQQQFQQQQQQQQMGLLNQSRTLLSPQQQQQQQVALGPGMPAKPLQHFSSPGALGPTLLLTGKEQNTVDPAVSSEATEGPSTHQGGPLAIGTTPESMATEPGEVKPSLSGDSQLLLVQPQPQPQPSSLQLQPPLRLPGQQQQQVSLLHTAGGGSHGQLGSGSSSEASSVPHLLAQPSVSLGDQPGSMTQNLLGPQQPMLERPMQNNTGPQPPKPGPVLQSGQGLPGVGIMPTVGQLRAQLQGVLAKNPQLRHLSPQQQQQLQALLMQRQLQQSQAVRQTPPYQEPGTQTSPLQGLLGCQPQLGGFPGPQTGPLQELGAGPRPQGPPRLPAPPGALSTGPVLGPVHPTPPPSSPQEPKRPSQLPSPSSQLPTEAQLPPTHPGTPKPQGPTLEPPPGRVSPAAAQLADTLFSKGLGPWDPPDNLAETQKPEQSSLVPGHLDQVNGQVVPEASQLSIKQEPREEPCALGAQSVKREANGEPIGAPGTSNHLLLAGPRSEAGHLLLQKLLRAKNVQLSTGRGSEGLRAEINGHIDSKLAGLEQKLQGTPSNKEDAAARKPLTPKPKRVQKASDRLVSSRKKLRKEDGVRASEALLKQLKQELSLLPLTEPAITANFSLFAPFGSGCPVNGQSQLRGAFGSGALPTGPDYYSQLLTKNNLSNPPTPPSSLPPTPPPSVQQKMVNGVTPSEELGEHPKDAASARDSERALRDTSEVKSLDLLAALPTPPHNQTEDVRMESDEDSDSPDSIVPASSPESILGEEAPRFPHLGSGRWEQEDRALSPVIPLIPRASIPVFPDTKPYGALGLEVPGKLPVTTWEKGKGSEVSVMLTVSAAAAKNLNGVMVAVAELLSMKIPNSYEVLFPESPARAGTEPKKGEAEGPGGKEKGLEGKSPDTGPDWLKQFDAVLPGYTLKSQLDILSLLKQESPAPEPPTQHSYTYNVSNLDVRQLSAPPPEEPSPPPSPLAPSPASPPTEPLVELPTEPLAEPPVPSPLPLASSPESARPKPRARPPEEGEDSRPPRLKKWKGVRWKRLRLLLTIQKGSGRQEDEREVAEFMEQLGTALRPDKVPRDMRRCCFCHEEGDGATDGPARLLNLDLDLWVHLNCALWSTEVYETQGGALMNVEVALHRGLLTKCSLCQRTGATSSCNRMRCPNVYHFACAIRAKCMFFKDKTMLCPMHKIKGPCEQELSSFAVFRRVYIERDEVKQIASIIQRGERLHMFRVGGLVFHAIGQLLPHQMADFHSATALYPVGYEATRIYWSLRTNNRRCCYRCSIGENNGRPEFVIKVIEQGLEDLVFTDASPQAVWNRIIEPVAAMRKEADMLRLFPEYLKGEELFGLTVHAVLRIAESLPGVESCQNYLFRYGRHPLMELPLMINPTGCARSEPKILTHYKRPHTLNSTSMSKAYQSTFTGETNTPYSKQFVHSKSSQYRRLRTEWKNNVYLARSRIQGLGLYAAKDLEKHTMVIEYIGTIIRNEVANRREKIYEEQNRGIYMFRINNEHVIDATLTGGPARYINHSCAPNCVAEVVTFDKEDKIIIISSRRIPKGEELTYDYQFDFEDDQHKIPCHCGAWNCRKWMN

SEQ ID NO: 49 - DNA, 4811 bp, Homo sapiens
agtggcgtcggaactgcaaagcacctgtgagcttgcggaagtcagttcagactccagcccgctccagcccggcccgacccgaccgcacccggcgcctgccctcgctcggcgtccccggccagccatgggcccttggagccgcagcctctcggcgctgctgctgctgctgcaggtctcctcttggctctgccaggagccggagccctgccaccctggctttgacgccgagagctacacgttcacggtgccccggcgccacctggagagaggccgcgtcctgggcagagtgaattttgaagattgcaccggtcgacaaaggacagcctatttttccctcgacacccgattcaaagtgggcacagatggtgtgattacagtcaaaaggcctctacggtttcataacccacagatccatttcttggtctacgcctgggactccacctacagaaagttttccaccaaagtcacgctgaatacagtggggcaccaccaccgccccccgccccatcaggcctccgtttctggaatccaagcagaattgctcacatttcccaactcctctcctggcctcagaagacagaagagagactgggttattcctcccatcagctgcccagaaaatgaaaaaggcccatttcctaaaaacctggttcagatcaaatccaacaaagacaaagaaggcaaggttttctacagcatcactggccaaggagctgacacaccccctgttggtgtctttattattgaaagagaaacaggatggctgaaggtgacagagcctctggatagagaacgcattgccacatacactctcttctctcacgctgtgtcatccaacgggaatgcagttgaggatccaatggagattttgatcacggtaaccgatcagaatgacaacaagcccgaattcacccaggaggtctttaaggggtctgtcatggaaggtgctcttccaggaacctctgtgatggaggtcacagccacagacgcggacgatgatgtgaacacctacaatgccgccatcgcttacaccatcctcagccaagatcctgagctccctgacaaaaatatgttcaccattaacaggaacacaggagtcatcagtgtggtcaccactgggctggaccgagagagtttccctacgtataccctggtggttcaagctgctgaccttcaaggtgaggggttaagcacaacagcaacagctgtgatcacagtcactgacaccaacgataatcctccgatcttcaatcccaccacgtacaagggtcaggtgcctgagaacgaggctaacgtcgtaatcaccacactgaaagtgactgatgctgatgcccccaataccccagcgtgggaggctgtatacaccatattgaatgatgatggtggacaatttgtcgtcaccacaaatccagtgaacaacgatggcattttgaaaacagcaaagggcttggattttgaggccaagcagcagtacattctacacgtagcagtgacgaatgtggtaccttttgaggtctctctcaccacctccacagccaccgtcaccgtggatgtgctggatgtgaatgaagcccccatctttgtgcctcctgaaaagagagtggaagtgtccgaggactttggcgtgggccaggaaatcacatcctacactgcccaggagccagacacatttatggaacagaaaataacatatcggatttggagagacactgccaactggctggagattaatccggacactggtgccatttccactcgggctgagctggacagggaggattttgagcacgtgaagaacagcacgtacacagccctaatcatagctacagacaatggttctccagttgctactggaacagggacacttctgctgatcctgtctgatgtgaatgacaacgcccccataccagaacctcgaactatattcttctgtgagaggaatccaaagcctcaggtcataaacatcattgatgcagaccttcctcccaatacatctcccttcacagcagaactaacacacggggcgagtgccaactggaccattcagtacaacgacccaacccaagaatctatcattttgaagccaaagatggccttagaggtgggtgactacaaaatcaatctcaagctcatggataaccagaataaagaccaagtgaccaccttagaggtcagcgtgtgtgactgtgaaggggccgctggcgtctgtaggaaggcacagcctgtcgaagcaggattgcaaattcctgccattctggggattcttggaggaattcttgctttgctaattctgattctgctgctcttgctgtttcttcggaggagagcggtggtcaaagagcccttactgcccccagaggatgacacccgggacaacgtttattactatgatgaagaaggaggcggagaagaggaccaggactttgacttgagccagctgcacaggggcctggacgctcggcctgaagtgactcgtaacgacgttgcaccaaccctcatgagtgtcccccggtatcttccccgccctgccaatcccgatgaaattggaaattttattgatgaaaatctgaaagcggctgatactgaccccacagccccgccttatgattctctgctcgtgtttgactatgaaggaagcggttccgaagctgctagtctgagctccctgaactcctcagagtcagacaaagaccaggactatgactacttgaacgaatggggcaatcgcttcaagaagctggctgacatgtacggaggcggcgaggacgactaggggactcgagagaggcgggccccagacccatgtgctgggaaatgcagaaatcacgttgctggtggtttttcagctcccttcccttgagatgagtttctggggaaaaaaaagagactggttagtgatgcagttagtatagctttatactctctccactttatagctctaataagtttgtgttagaaaagtttcgacttatttcttaaagcttttttttttttcccatcactctttacatggtggtgatgtccaaaagatacccaaattttaatattccagaagaacaactttagcatcagaaggttcacccagcaccttgcagattttcttaaggaattttgtctcacttttaaaaagaaggggagaagtcagctactctagttctgttgttttgtgtatataattttttaaaaaaaatttgtgtgcttctgctcattactacactggtgtgtccctctgccttttttttttttttaagacagggtctcattctatcggccaggctggagtgcagtggtgcaatcacagctcactgcagccttgtcctcccaggctcaagctatccttgcacctcagcctcccaagtagctgggaccacaggcatgcaccactacgcatgactaattttttaaatatttgagacggggtctccctgtgttacccaggctggtctcaaactcctgggctcaagtgatcctcccatcttggcctcccagagtattgggattacagacatgagccactgcacctgcccagctccccaactccctgccattttttaagagacagtttcgctccatcgcccaggcctgggatgcagtgatgtgatcatagctcactgtaacctcaaactctggggctcaagcagttctcccaccagcctcctttttatttttttgtacagatggggtcttgctatgttgcccaagctggtcttaaactcctggcctcaagcaatccttctgccttggccccccaaagtgctgggattgtgggcatgagctgctgtgcccagcctccatgttttaatatcaactctcactcctgaattcagttgctttgcccaagataggagttctctgatgcagaaattattgggctcttttagggtaagaagtttgtgtctttgtctggccacatcttgactaggtattgtctactctgaagacctttaatggcttccctctttcatctcctgagtatgtaacttgcaatgggcagctatccagtgacttgttctgagtaagtgtgttcattaatgtttatttagctctgaagcaagagtgatatactccaggacttagaatagtgcctaaagtgctgcagccaaagacagagcggaactatgaaaagtgggcttggagatggcaggagagcttgtcattgagcctggcaatttagcaaactgatgctgaggatgattgaggtgggtctacctcatctctgaaaattctggaaggaatggaggagtctcaacatgtgtttctgacacaagatccgtggtttgtactcaaagcccagaatccccaagtgcctgcttttgatgatgtctacagaaaatgctggctgagctgaacacatttgcccaattccaggtgtgcacagaaaaccgagaatattcaaaattccaaatttttttcttaggagcaagaagaaaatgtggccctaaagggggttagttgaggggtagggggtagtgaggatcttgatttggatctctttttatttaaatgtgaatttcaacttttgacaatcaaagaaaagacttttgttgaaatagctttactgtttctcaagtgttttggagaaaaaaatcaaccctgcaatcactttttggaattgtcttgatttttcggcagttcaagctatatcgaatatagttctgtgtagagaatgtcactgtagttttgagtgtatacatgtgtgggtgctgataattgtgtattttctttgggggtggaaaaggaaaacaattcaagctgagaaaagtattctcaaagatgcatttttataaattttattaaacaattttgttaaa

SEQ ID NO: 50 - protein, 882 aa, Homo sapiens
MGPWSRSLSALLLLLQVSSWLCQEPEPCHPGFDAESYTFTVPRRHLERGRVLGRVNFEDCTGRQRTAYFSLDTRFKVGTDGVITVKRPLRFHNPQIHFLVYAWDSTYRKFSTKVTLNTVGHHHRPPPHQASVSGIQAELLTFPNSSPGLRRQKRDWVIPPISCPENEKGPFPKNLVQIKSNKDKEGKVFYSITGQGADTPPVGVFIIERETGWLKVTEPLDRERIATYTLFSHAVSSNGNAVEDPMEILITVTDQNDNKPEFTQEVFKGSVMEGALPGTSVMEVTATDADDDVNTYNAAIAYTILSQDPELPDKNMFTINRNTGVISVVTTGLDRESFPTYTLVVQAADLQGEGLSTTATAVITVTDTNDNPPIFNPTTYKGQVPENEANVVITTLKVTDADAPNTPAWEAVYTILNDDGGQFVVTTNPVNNDGILKTAKGLDFEAKQQYILHVAVTNVVPFEVSLTTSTATVTVDVLDVNEAPIFVPPEKRVEVSEDFGVGQEITSYTAQEPDTFMEQKITYRIWRDTANWLEINPDTGAISTRAELDREDFEHVKNSTYTALIIATDNGSPVATGTGTLLLILSDVNDNAPIPEPRTIFFCERNPKPQVINIIDADLPPNTSPFTAELTHGASANWTIQYNDPTQESIILKPKMALEVGDYKINLKLMDNQNKDQVTTLEVSVCDCEGAAGVCRKAQPVEAGLQIPAILGILGGILALLILILLLLLFLRRRAVVKEPLLPPEDDTRDNVYYYDEEGGGEEDQDFDLSQLHRGLDARPEVTRNDVAPTLMSVPRYLPRPANPDEIGNFIDENLKAADTDPTAPPYDSLLVFDYEGSGSEAASLSSLNSSESDKDQDYDYLNEWGNRFKKLADMYGGGEDD

SEQ ID NO: 51 - protein, 4 aa, Homo sapiens
YVMA

SEQ ID NO: 52 - protein, 3 aa, Homo sapiens
GSP